(19) United States
(12) Patent Application Publication
(10) Pub. No.: US 2023/0227838 A1
(43) Pub. Date: Jul. 20, 2023

(54) ENHANCED CELL SURVIVAL AGAINST BIOTIC AND ABIOTIC STRESSES THROUGH SALICYLICK ACID-INDUCED NPR1 CONDENSATES

(71) Applicant: Duke University, Durham, NC (US)

(72) Inventors: Xinnian DONG, Durham, NC (US); Raul ZAVALIEV, Durham, NC (US)

(21) Appl. No.: 18/001,884

(22) PCT Filed: Jun. 22, 2021
(86) PCT No.: PCT/US2021/038430

§ 371 Date: Dec. 15, 2022

Related U.S. Application Data
(60) Provisional application No. 63/042,237, filed on Jun. 22, 2020.

(51) Int. Cl. C12N 15/82; C07K 14/415

(52) U.S. Cl. CPC C12N 15/8271; C07K 14/415

(57) ABSTRACT

The present disclosure describes compositions and methods for promoting cell survival against biotic and abiotic stress and during plant immune responses.

### CROSS-REFERENCE TO RELATED APPLICATIONS

This application claims the benefit of U.S. Provisional Application No. 63/042,237, filed Jun. 22, 2020, which is incorporated herein by reference.

### FEDERAL FUNDING LEGEND

This invention was made with Government support under Federal Grant no. R35GM118036 awarded by the National Institutes of Health. The Federal Government has certain rights to this invention.

### SEQUENCE LISTING

The Sequence Listing written in file 560198_SeqListing_ST25.txt is 257 kilobytes in size, was created Jun. 15, 2021, and is hereby incorporated by reference.

### BACKGROUND

An essential feature of immunity is to ensure defense against pathogens without collateral damage to self. In plants, pathogen effector-triggered immunity (ETI) often results in programmed cell death, which is restricted through the function of NPR1, a positive regulator of systemic acquired resistance. Despite the importance of this protein and the extent to which it has been studied, the biochemical activities of NPR1 that promote both defense and cell survival remain unclear.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure provides, is based, in part on the discovery by the inventors that NPR1 promotes cell survival by targeting substrates for ubiquitination and degradation through salicylic acid (SA)-driven phase separation into cytoplasmic condensates. Further, the inventors show that NPR1 condensates are enriched in cell death regulators including nucleotide-binding leucine-rich repeat immune receptors, redox metabolism proteins, DNA damage repair and protein quality control machineries. Phase separation of NPR1 is required for recruitment of the Cullin 3 RING E3 ligase complex to the condensates and NPR1 can promote cell survival by degrading EDS1 and specific WRKY transcription factors required for ETI. The discovery of distinct functional groups of proteins in the SA-induced NPR1 condensates, herein termed “SINCs”, suggests that NPR1 modulates survival by converging multiple stress-responsive processes in this quasi organelle.

Other aspects and embodiments of the present disclosure provide compositions and methods for promoting cell survival of a cell comprising, consisting of, or consisting essentially of modulating NPR1 in the cell. In some embodiments, modulating may comprise upregulating the expression of and/or enhancing the function of, NPR1 in the cell. In another embodiment, the cell comprises a plant cell.

In one aspect, provided are nucleic acids encoding a npr1 protein, wherein the npr1 protein forms salicylic acid-independent NPR1 condensates. In some such nucleic acids, the nucleic acid is operably linked to one or more expression control elements. In some such nucleic acids, the one or more expression control elements comprise: a promoter, one or more upstream open reading frames (uORFs), or the promoter and the one or more uORFs. In some such nucleic acids, the promoter is selected from the group consisting of: a constitutive promoter, an inducible promoter, a temporally-regulated promoter, a developmentally regulated promoter, a chemically regulated promoter, a tissue-preferred promoter, a tissue-specific promoter, a TBF1 promoter, a 35S promoter, a ubiquitin promoter, a tCUP cryptic constitutive promoter, a Rsyn7 promoter, a pathogen-inducible promoter, a maize In2-2 promoter, a tobacco PR-1a promoter, a glucocorticoid-inducible promoter, an estrogen-inducible promoter, a tetracycline-inducible promoter, a tetracycline-repressible promoter, a T3 promoter, a T7 promoter, and a SP6 promoter. In some such nucleic acids, the promoter is the TBF1 promoter. In some such nucleic acids, the uORF comprises a TBF1 gene uORF. In some such nucleic acids, the expression control elements comprise the TBF1 promoter and one or more TBF1 uORFs.

In some such nucleic acids, the npr1 protein is an A. thaliana npr1 protein comprising one or more mutations. In some such nucleic acids, the npr1 protein comprises one or more mutations in at least one redox-sensitive intrinsically disordered region (RDR). In some such nucleic acids, the one or more mutations comprises a substitution of one or more cysteines in at least one RDR, a deletion of one or more cysteines in at least one RDR, or a combination thereof. In some such nucleic acids, the one or more cysteines are located in a region corresponding to residues 140-160, 368-404, or 510-539 of SEQ ID NO: 1. In some such nucleic acids, the one or more cysteines are located in the region corresponding to residues 368-404 of SEQ ID NO: 1. In some such nucleic acids, the npr1 protein has increased interaction with CUL3 compared to wild-type NPR1 in the absence of salicylic acid. In some such nucleic acids, the one or more mutations reduces the redox-sensitivity of the RDR. In some such nucleic acids, the npr1 protein comprises a mutation of a cysteine corresponding to the cysteine at position 378, of SEQ ID NO: 1, position 385 of SEQ ID NO: 1, position 394 of SEQ ID NO: 1, positions 378 and 385 of SEQ ID NO: 1, positions 378 and 394 of SEQ ID NO: 1, positions 385 and 394 of SEQ ID NO: 1, or positions 378, 385, and 394 of SEQ ID NO: 1. In some such nucleic acids, the npr1 protein comprises the mutation of the cysteine corresponding to the cysteines at positions 378, 385, and 394 of SEQ ID NO: 1. In some such nucleic acids, the mutation of the cysteine comprises an alanine substitution. In some such nucleic acids, the npr1 protein comprises a mutation of one or more cysteines corresponding to the cysteines at positions 150, 155, 156, and 160 of SEQ ID NO: 1.

In some such nucleic acids, the nucleic acid encodes a ΔCTD npr1 ACTD protein, a BTB domain npr1 protein, or a sim3 npr1 protein.

In some such nucleic acids, the nucleic acid encodes: (a) a protein comprising the amino acid sequence of any of SEQ ID NOS: 134-160 or an ortholog thereof; or (b) a protein having at least 70% identity to a protein comprising the amino acid sequence of any of SEQ ID NOS: 134-160. In some such nucleic acids, the nucleic acid encodes a protein comprising the amino acid sequence of SEQ ID NO: 134.

In another aspect, provided are plants or plant cells expressing any of the above nucleic acids. In some such plants or plant cells, the plant is a monocot or a dicot. In some such plants or plant cells, the plant is a food crop plant, a biofuel plant, a corn plant, a legume plant, a bean plant, a rice plant, a soybean plant, a cotton plant, a sugarcane plant, a tobacco plant, a palm oil plant, a date palm, a wheat, a vegetable plant, a squash plant, a Solanaceae plant, a tomato, a banana plant, a potato plant, a pepper plant, a moss plant, a parsley plant, a sunflower plant, a mustard plant, a sorghum plant, a millet plant, a citrus plant, an apple plant, a strawberry plant, a rapeseed plant, a cabbage plant, a cassava plant, a coffee plant, a sweet potato plant, a jatropha plant, or a switchgrass plant. In some such plants or plant cells, the plant expresses a wild-type NPR1 gene. In some such plants or plant cells, the plant does not express a wild-type NPR1 gene.

In another aspect, provided are methods of increasing stress tolerance in a plant, comprising expressing in the plant any of the above nucleic acids. In some such methods, the stress is biotic or abiotic stress. In some such methods, the biotic stress comprises pathogen infection. In some such methods, the pathogen is a bacteria or a virus. In some such methods, the abiotic stress comprises high temperature (heat shock) stress, low temperature (cold shock) stress, oxidative stress, or DNA damage. In some such methods, increasing stress tolerance comprises one or more of: decreasing programmed cell death, decreasing effector-triggered immunity (ETI)-induced cell death, increasing formation of NPR1 condensates, and degrading EDS1 and specific WRKY transcription factors required for pathogen ETI. In some such methods, the plant is a monocot or a dicot. In some such methods, the plant is a food crop plant, a biofuel plant, a corn plant, a legume plant, a bean plant, a rice plant, a soybean plant, a cotton plant, a sugarcane plant, a tobacco plant, a palm oil plant, a date palm, a wheat, a vegetable plant, a squash plant, a Solanaceae plant, a tomato, a banana plant, a potato plant, a pepper plant, a moss plant, a parsley plant, a sunflower plant, a mustard plant, a sorghum plant, a millet plant, a citrus plant, an apple plant, a strawberry plant, a rapeseed plant, a cabbage plant, a cassava plant, a coffee plant, a sweet potato plant, a jatropha plant, or a switchgrass plant. In some such methods, the plant expresses a wild-type NPR1 gene. In some such methods, the plant does not express a wild-type NPR1 gene.

Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which:

FIGS. 1A-1K show NPR1 is required for cell survival and accumulation of ubiquitinated proteins. In FIGS. 1A-1C, half leaves (left side) of Col-0, npr1-2 and sid2-2 plants were infiltrated with (mock) or Psm ES4326/AvrRpt2 (Avr). At 2 dpi, the adjacent leaf halves were infiltrated with the same pathogen. Cell death was assessed by tissue collapse at 1 dpi (FIG. 1A) and conductivity assay (FIG. 1B). Growth of Psm ES4326/AvrRpt2 (Tetr) was measured in the adjacent leaf halves after the first inoculation with Psm ES4326/AvrRpm1 (Avr; Kanr) (FIG. 1C). Data are presented as mean±SD (FIG. 1B), and mean±95% confidence intervals (FIG. 1C). In FIG. 1D, half leaves of plants expressing dex:AvrRpt2 in Col-0, npr1-2, and rps2 were inoculated as in FIG. 1B. At 2 dpi, the adjacent leaf halves were infiltrated with 25 mM dexamethasone (dex) and cell death was assessed as in FIG. 1B. Data are presented as mean±SD. In FIGS. 1E-1G, Col-0, rps2 and npr1-2 plants were treated with water (mock) or 1 mM SA 24 hr before inoculation with Psm ES4326/AvrRpt2. Cell death was assessed by trypan blue staining (FIG. 1E) and conductivity assay (FIG. 1F). Bacterial growth was measured at 1 dpi (FIG. 1G). Data are presented as mean±SD (FIG. 1F), and mean±95% confidence intervals (FIG. 1G). In FIG. 1H, plants expressing dex:AvrRpt2 in Col-0, npr1-2 and rps2 were treated as in FIGS. 1E-1G before treatment with 25 μM dexamethasone (dex). Cell death was assessed as in FIG. 1B. Data are presented as mean±SD. In FIGS. 1I-1J, Ws-2 (FIG. 1I) or Col-0 and npr1-2 (FIG. 1J), were treated as in FIGS. 1E-1G before inoculation with Pf Pf0-1/AvrRps4 or Pf Pf0-1/AvrRps4KRVY-AAAA. Cell death was assessed as in FIG. 1B. Data are presented as mean±SD. In FIG. 1K, Col-0 and npr1-2 plants were treated with SA for 6 hr. Total proteins were probed with α-Ub and α-NPR1. See also FIGS. 8A-8E.

FIGS. 2A-2I show NPR1 accumulates in the cytoplasm and forms cysteine-dependent condensates. FIG. 2A shows subcellular fractionation of Col-0 after 6 hr SA treatment. Cytoplasmic (C), nuclear (N) and combined (C+N) fractions were probed with α-NPR1 and α-Ub antibodies. Band intensities (b.i.)in the upper blot are shown as percentages of the combined C+N levels. The middle blot (a longer exposure of the upper blot) shows the higher MW NPR1 bands. FIG. 2B shows localization of NPR1-GFP and sim3-GFP in transient expression in Col-0 seedlings treated with SA for 2 hr. Scale bar=20 μm. FIGS. 2C-2E show single-cell time-lapse imaging of NPR1-GFP condensation induced by 5 mM SA. Imaging was started 40 min after SA induction (time 0) for a duration of 2 hr. Images from selected time points (FIG. 2C), fluorescence intensity per body (FIG. 2D) and number of bodies (FIG. 2E) are shown. Scale bar=20 μm. Data are presented as mean±SE (FIG. 2D). FIG. 2F shows predicted RDR regions of NPR1. Values represent differential IDR score. Dots indicate position of cysteine residues (red), and known point mutations and their alleles (black). FIGS. 2G and 2H show localization of NPR1-GFP and rdr3-GFP after treatment with 5 mM SA for 2 hr (FIG. 2G). Scale bar=20 μm. Insets show enlarged nuclei at lower exposure. FIG. 2H shows total fluorescence intensity of bodies from SA-treated samples. Data are presented as mean±SE. FIG. 2I shows transactivation of the PR1 promoter by NPR1 and rdr3 after treatment with 2 mM SA for 24 hr. Values represent the PR1 promoter activity plotted relative to free HA. Data are represented as mean±SD. See also FIGS. 8A-8E, 9A-9E and 10A-10I; Tables 3 and 4.

FIGS. 3A-3F show NPR1 condensates are enriched in stress proteins. FIG. 3A shows functional categorization of sim3-GFP interactome (SINC components). The relative sizes of functional groups (left) and the number of proteins at their intersection (right) are shown. FIG. 3B shows representative SINC components from four major functional groups. Black dots indicate confirmed localization in cytoplasmic NPR1 condensates. FIG. 3C shows co-localization of sim3-GFP with free mCherry or mCherry-fused SINC components, EDS1, BCS1, GSTU19 after treatment with 1 mM SA for 5 hr. Scale bar=20 μm. In FIGS. 3D-3F, Col-0 and npr1-2 plants were treated with water (mock) or 1 mM SA 24 hr before inducing cell death with indicated stresses. Cell death was assessed by conductivity assay. Data are presented as mean±SD. See also FIGS. 11A-11H; Table 5.

FIGS. 4A-4L show NPR1 recruits CUL3 to its cytoplasmic condensates. FIG. 4A shows interaction of Myc-CUL3 with HA-fused NPR1 or its variants in N. benthamiana. Plants were treated with water (−) or 1 mM SA (+) for 5 hr before co-IP. FIG. 4B shows interaction of GFP-fused NPR1, sim3 and ABTB with the endogenous CUL3 in transgenic Arabidopsis. Plants were treated with 1 mM SA for 24 hr before co-IP. FIG. 4C shows interaction of Myc-CUL3 with GST-fused NPR1 or its variants in E. coli. Total protein from E. coli co-expressing Myc-CUL3 with either of GST-NPR1 variants or GST was used in the pull-down assay. FIG. 4D shows inhibition of CUL3-BTB interaction by the CTD in N. benthamiana. The three proteins, Myc-CUL3, BTB-HA and CTD-GFP, were co-expressed at 1:1:0 (0), 1:1:0.25 (1/4), 1:1:0.5 (1/2) and 1:1:1 (1) ratios, respectively, before co-IP. FIGS. 4E-4F show interaction of CUL3 with NPR1, sim3 or S55/59D in the BiFC assay after treatment with 1 mM SA for 5 hr. Scale bar=10 μm. BiFC signal intensity was quantified and values are plotted relative to CUL3/NPR1 (FIG. 4F). Data are presented as mean±SE. FIG. 4G shows localization of NPR1-GFP, sim3-GFP and S55/59D-GFP in N. benthamiana treated with water (mock) or 5 mM SA for 2 hr. Scale bar=10 μm. FIG. 4H shows interaction of Myc-CUL3 with HA-fused NPR1 or rdr mutants in N. benthamiana after treatment with 1 mM SA for 5 hr before co-IP. FIGS. 4I-4L show localization of GFP-CUL3 in NbNPR1-silenced N. benthamiana after treatment with 5 mM SA for 2 hr (FIG. 4I). Scale bar=20 μm. Total fluorescence intensity per body (FIG. 4J) and number of bodies per cell (FIG. 4K) were quantified relative to the E.V. control. Data are presented as mean±SE. Expression levels were verified by GFP-CUL3 immunoblotting (FIG. 4L). See also FIGS. 12A-12D and 13A-13F.

FIGS. 5A-5E show NPR1-CUL3 cytoplasmic condensates are active ubiquitination complexes. FIG. 5A shows co-localization of CUL3/sim3 BiFC bodies with mCherry-fused organelle and protein body markers in N. benthamiana treated with 1 mM SA for 5 hr SA. Scale bar=20 μm. Representative co-localizations with Ubiquitin and NBR1 are shown (left). FIG. 5B shows co-localization of sim3-GFP with mCherry-NBR1 in the presence of Myc-CUL3 or free Myc in N. benthamiana treated with 1 mM SA for 5 hr. Scale bar=20 μm. FIG. 5C shows interaction between Myc-CUL3 and HA-fused NPR1 or sim3 in N. benthamiana treated with SA for 5 hr before the pull-down assay (upper panels). Total ubiquitination was tested in the “input” fractions (lower panels). FIG. 5D shows total ubiquitination in N. benthamiana expressing Myc-CUL3 or Myc-CUL3ARBX1, sim3-GFP or GFP, and V5-Ub, after treatment with 1 mM SA for 5 hr. FIG. 5E shows total ubiquitination in the NbCUL3-silenced N. benthamiana or in the E.V. control expressing sim3-GFP and V5-Ub, after treatment with 1 mM SA for 5 hr. See also FIGS. 13A-13F.

FIGS. 6A-6I show SINC-localized proteins are targeted for degradation by NPR1-CRL3. FIG. 6A shows interaction of EDS1-mCherry with HA-fused NPR1 or sim3 in N. benthamiana after treatment with 1 mM SA for 5 hr before co-IP. FIG. 6B shows interaction of EDS1 with NPR1 in E. coli. Total protein from E. coli co-expressing FLAG-EDS1 with GST-NPR1 or GST was used for pull-down assay. FIG. 6C shows co-localization of NPR1/CUL3 or sim3/CUL3 BiFC bodies with EDS1-mCherry after treatment with 1 mM SA for 5 hr. Values quantified from free mCherry samples (not shown) are included in the quantification. Scale bar=20 μm. FIG. 6D shows stability of EDS1 in Col-0 or npr1-2 mutant. Seedlings were incubated in water (−) or 1 mM SA (+) for 4 hr, with (+) or without (−) subsequent addition of 100 μM cycloheximide (CHX). After 16 hours of co-incubation, total protein was probed with α-EDS1 and α-NPR1 antibodies. FIG. 6E shows ubiquitination of EDS1 in Col-0, npr1-2 and eds1-2 mutants treated with water (Mock) or 1 mM SA for 6 hr. FIG. 6F shows co-localization of sim3/CUL3 BiFC bodies with mCherry-fused WRKY54 or WRKY70 in N. benthamiana treated with 1 mM SA for 5 hr. Scale bar=20 μm. FIG. 6G shows stability of WRKY70-GFP in Col-0 or npr1-2 mutant. Seedlings were treated with 1 mM SA or 50 μM MG132 or in combination for 24 hr, and total protein was probed with α-GFP, α-NPR1 and α-TUB antibodies. FIG. 6H shows ubiquitination of WRKY70-GFP in Col-0 or npr1-2 mutant treated with 1 mM SA for 24 h before immunoprecipitation of WRKY70-GFP under denaturing conditions (dn). FIG. 6I shows NPR1- and sim3-dependent ubiquitination of FLAG-WRKY70 by the NPR1-CRL3 ubiquitination reconstituted in E. coli. FLAG-WRKY70 was immunoprecipitated under denaturing conditions (dn) using α-FLAG beads. See also FIGS. 14A-14D.

FIGS. 7A-7D show NPR1 promotes survival during ETI by targeting WRKY54 and WRKY70. In FIGS. 7A-7C, Col-0, rps2, npr1-2, wrky54 wrky70 (w54w70), and npr1 wrky54 wrky70 (n1w54w70) plants were treated with water (mock) or 1 mM SA 24 hr before inoculation with Psm ES4326/AvrRpt2. Cell death was assessed by trypan blue staining (FIG. 7A) and conductivity assay (FIG. 7B). Bacterial growth was measured at 1 dpi (FIG. 7C). Data are presented as mean±SD (FIG. 7B), and mean±95% confidence intervals (FIG. 7C). FIG. 7D shows a proposed model for NPR1 function in promoting cell survival during ETI. “P”, phosphorylation at S55/59; “S”, SUMOylation; “U”, ubiquitination; “W”, WRKY TFs; “NPR1C”, NPR1 condensate.

FIGS. 8A-8E show effect of SA and NPR1 on ETI-triggered cell death. Related to FIGS. 1A-1K and 2A-2I. In FIG. 8A, half leaves (left side) of plants expressing est:AvrRpt2 in Col-0, sid2-2 and rps2 backgrounds were infiltrated with MgSO4 (mock) or Psm ES4326/AvrRpm1

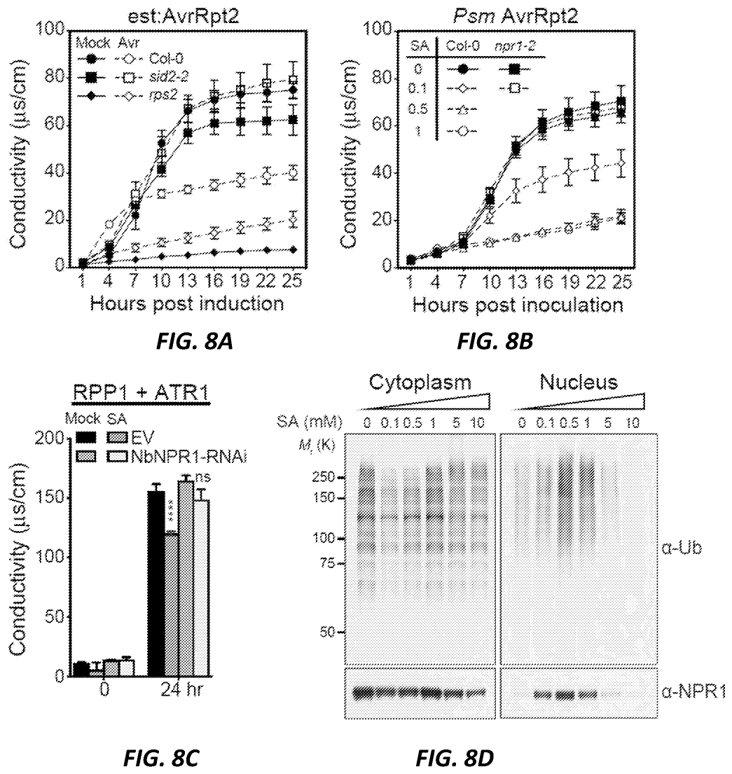

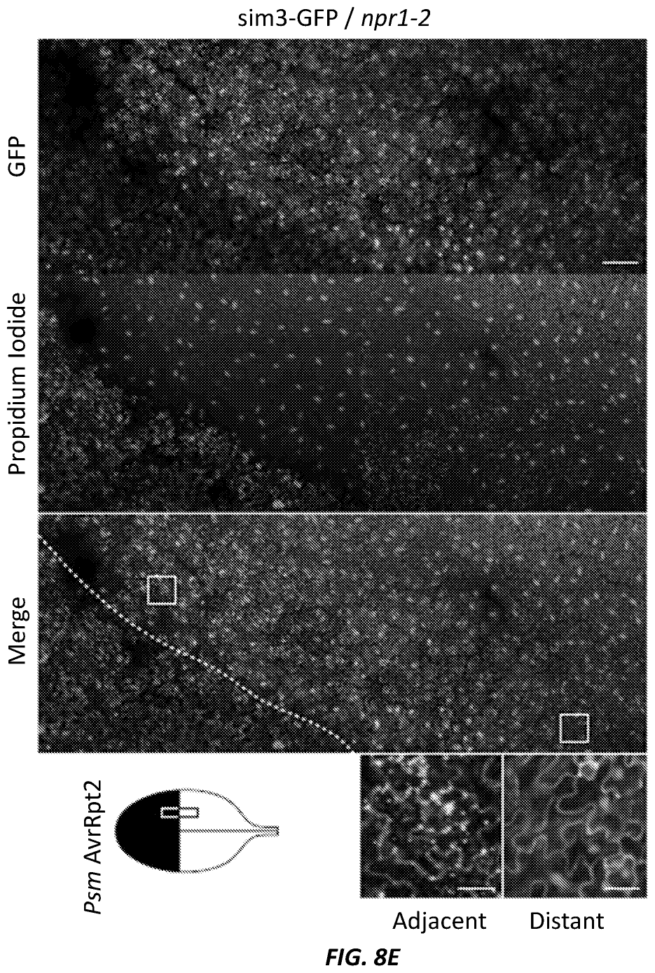

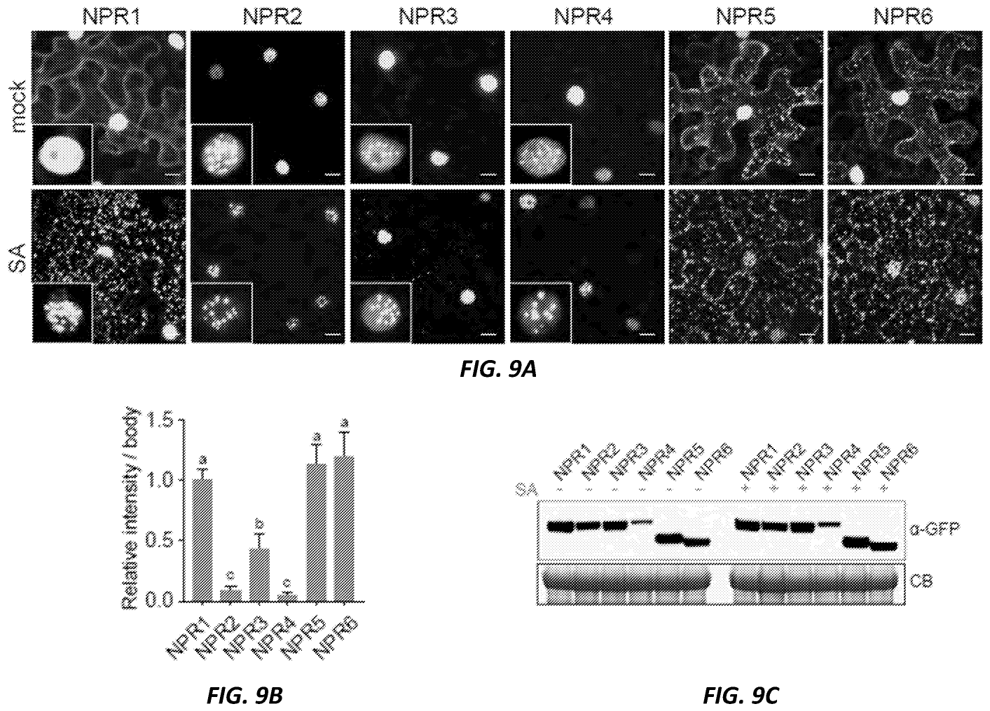

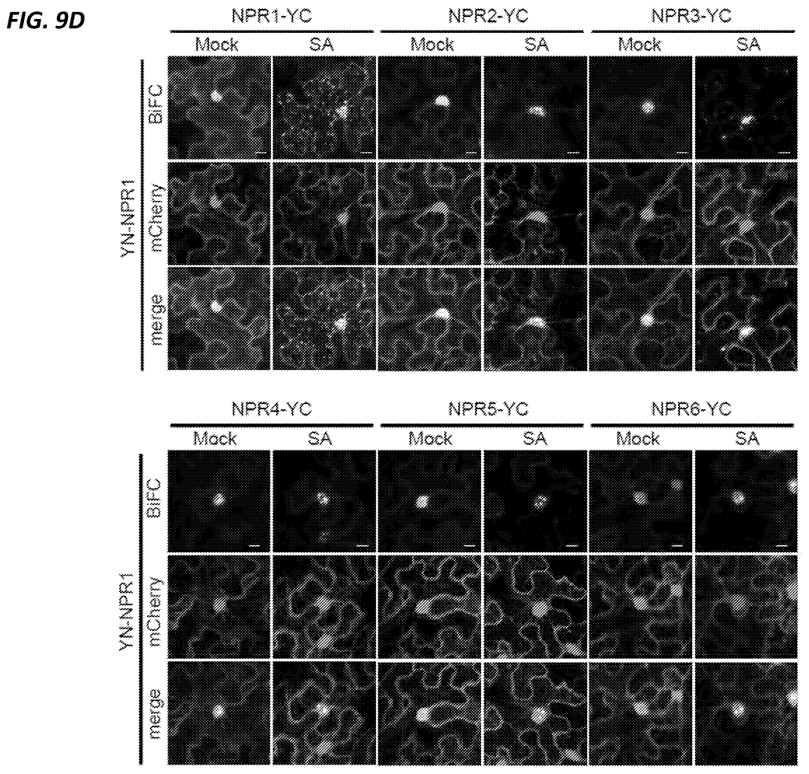

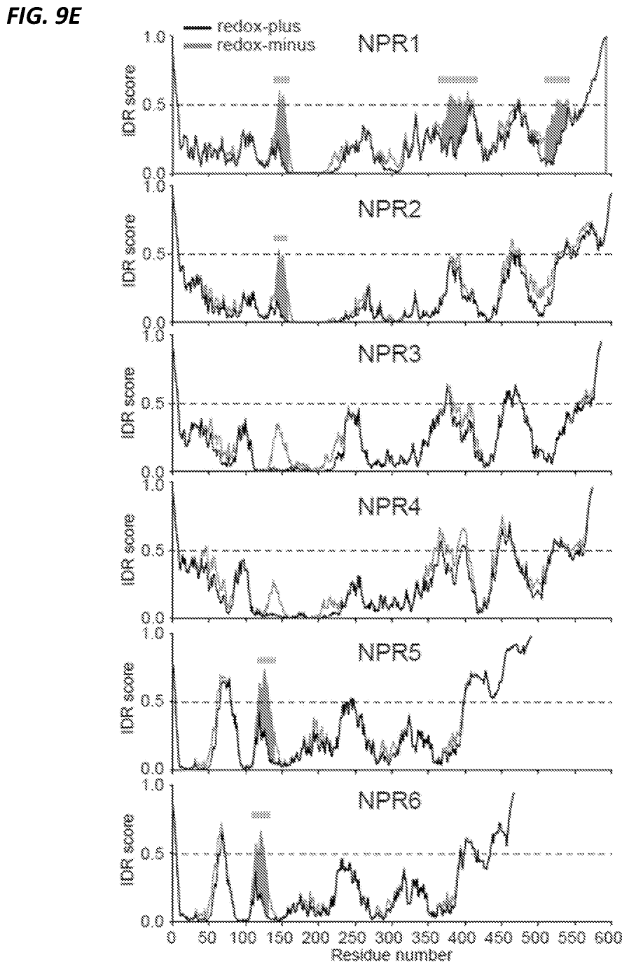

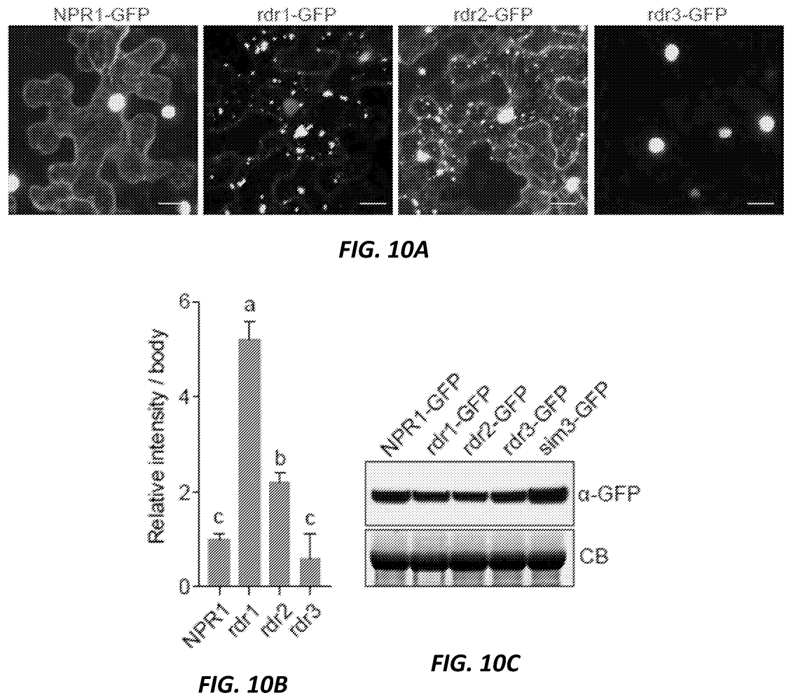

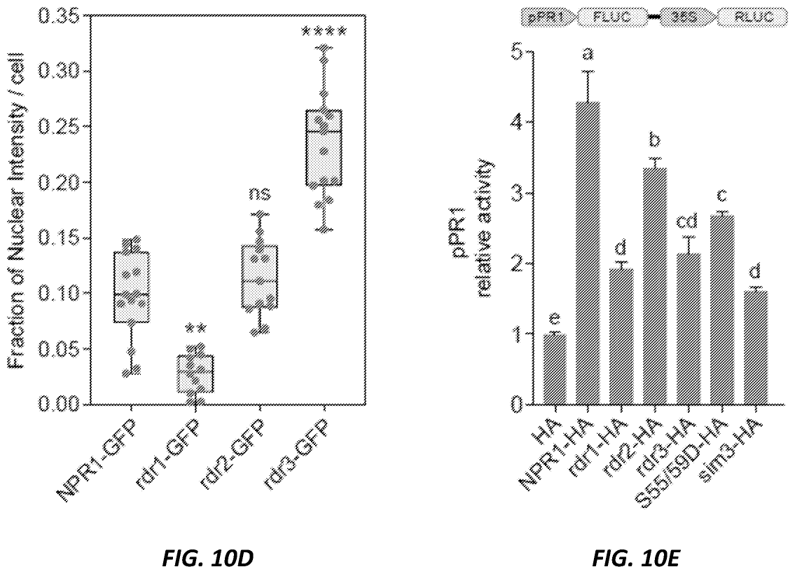

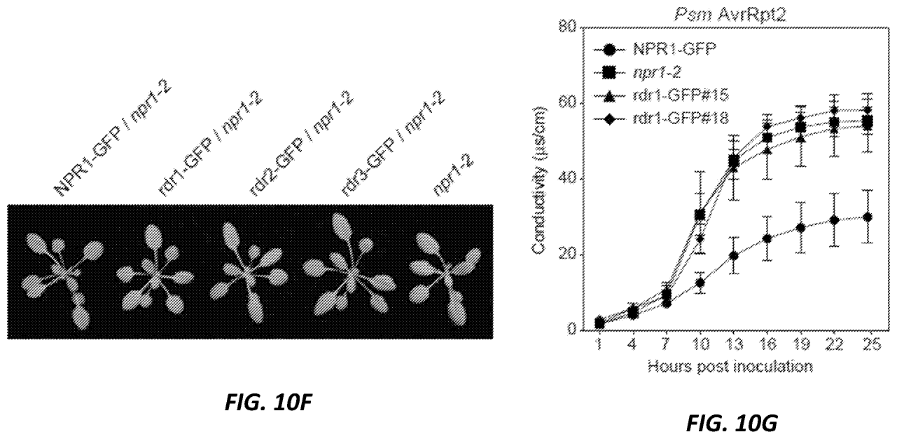

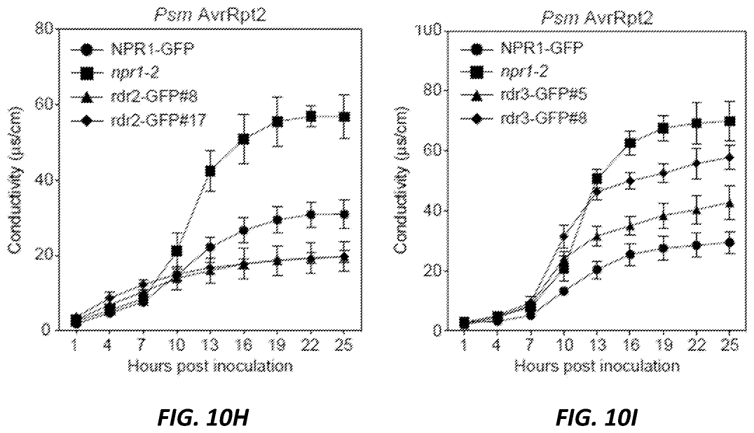

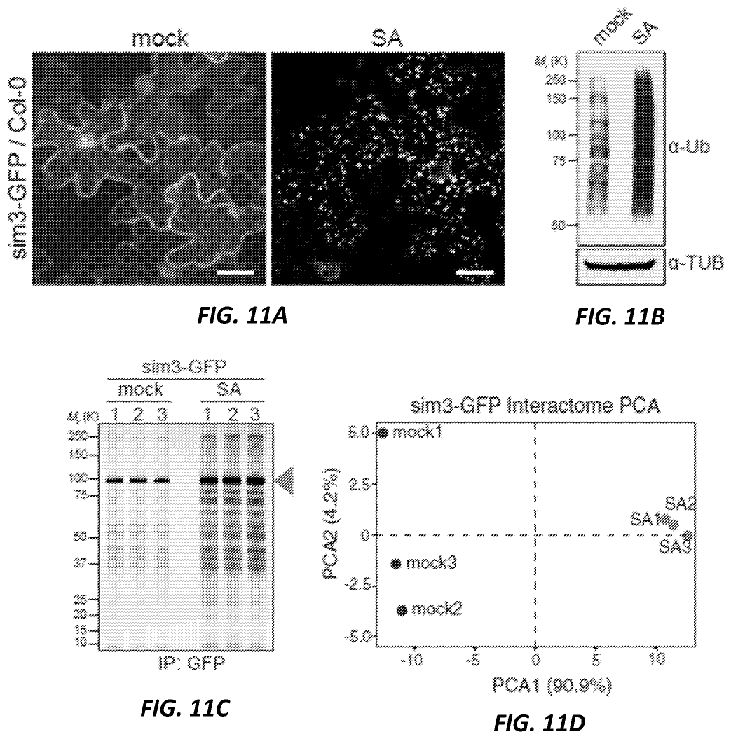

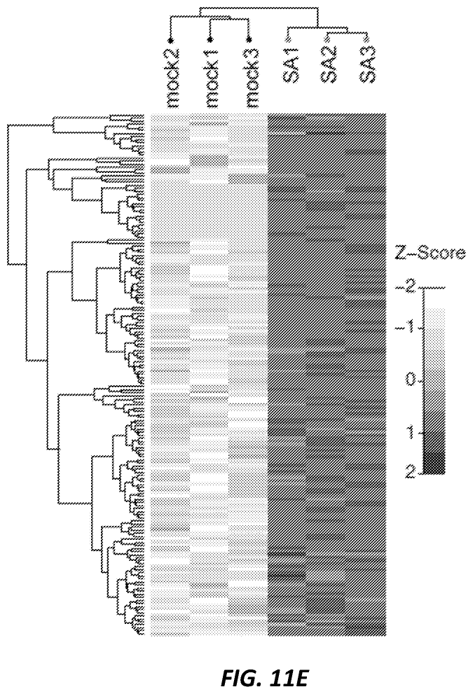

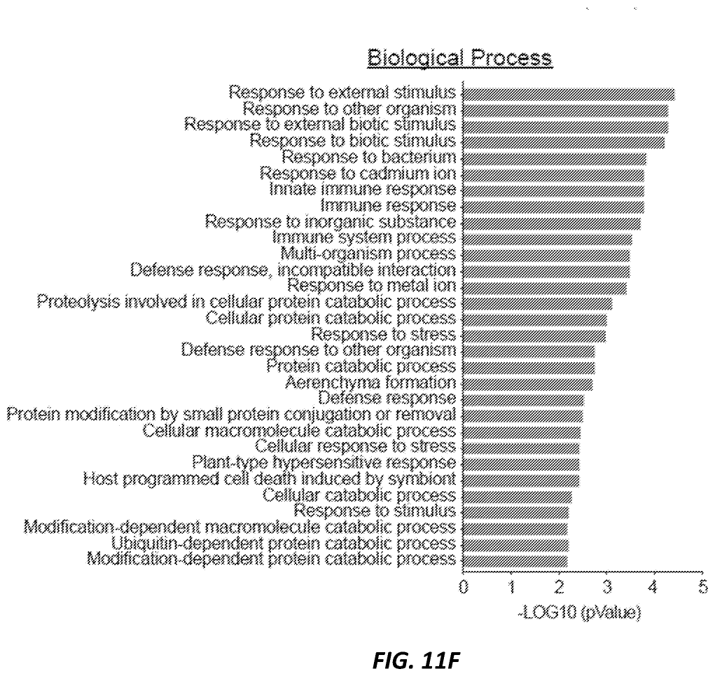

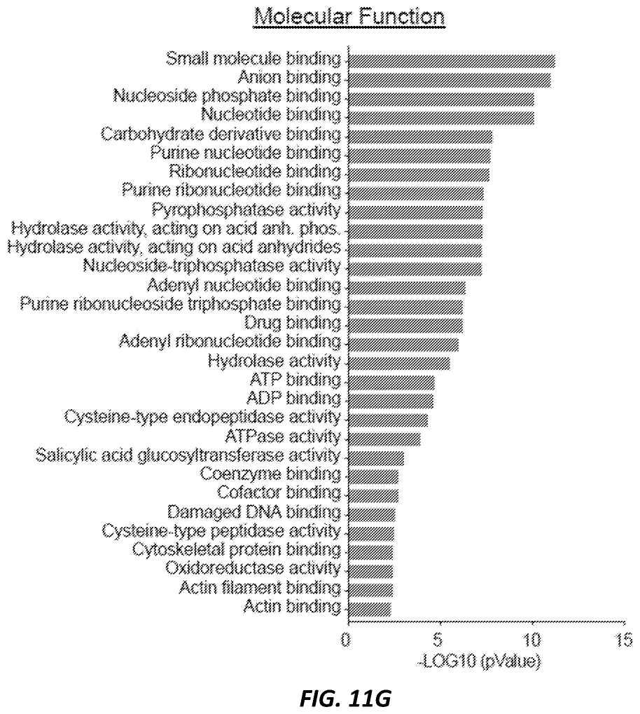

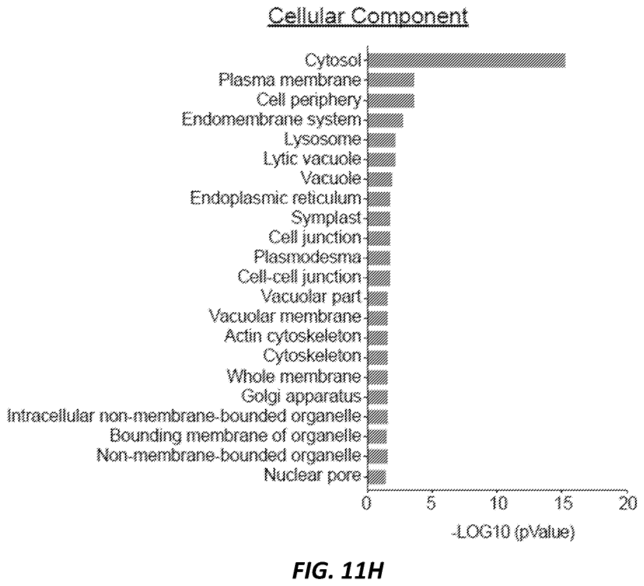

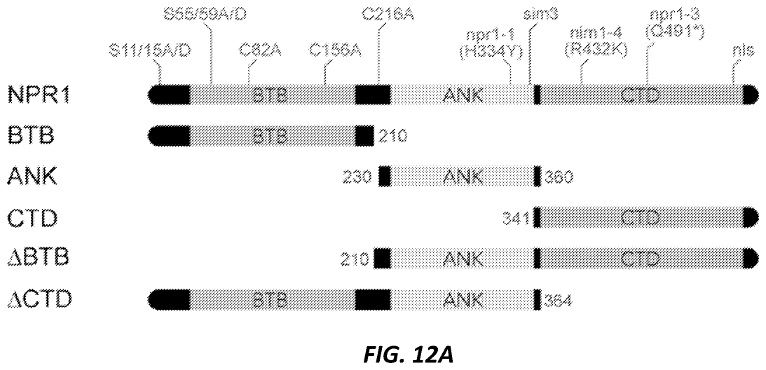

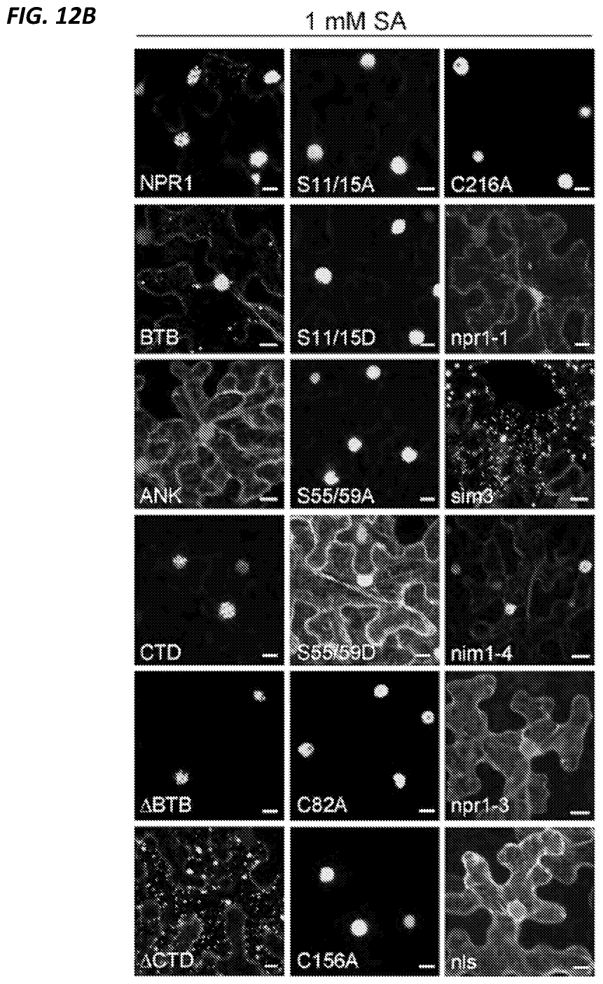

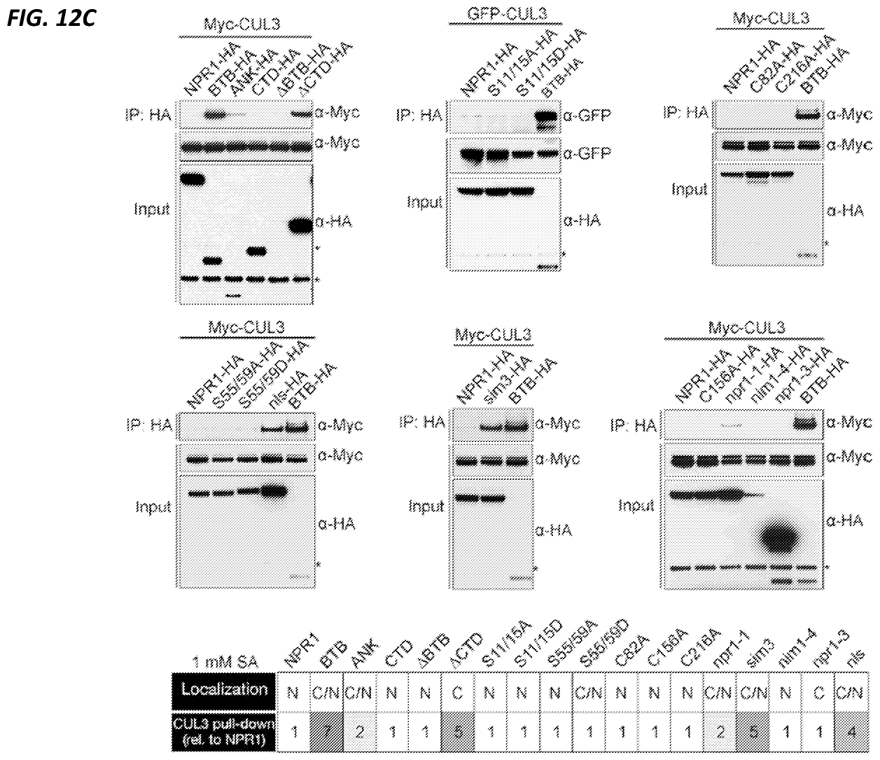

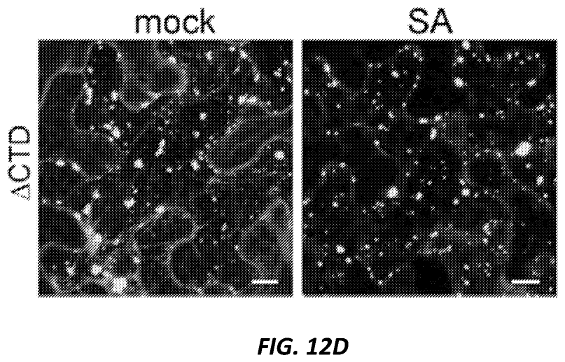

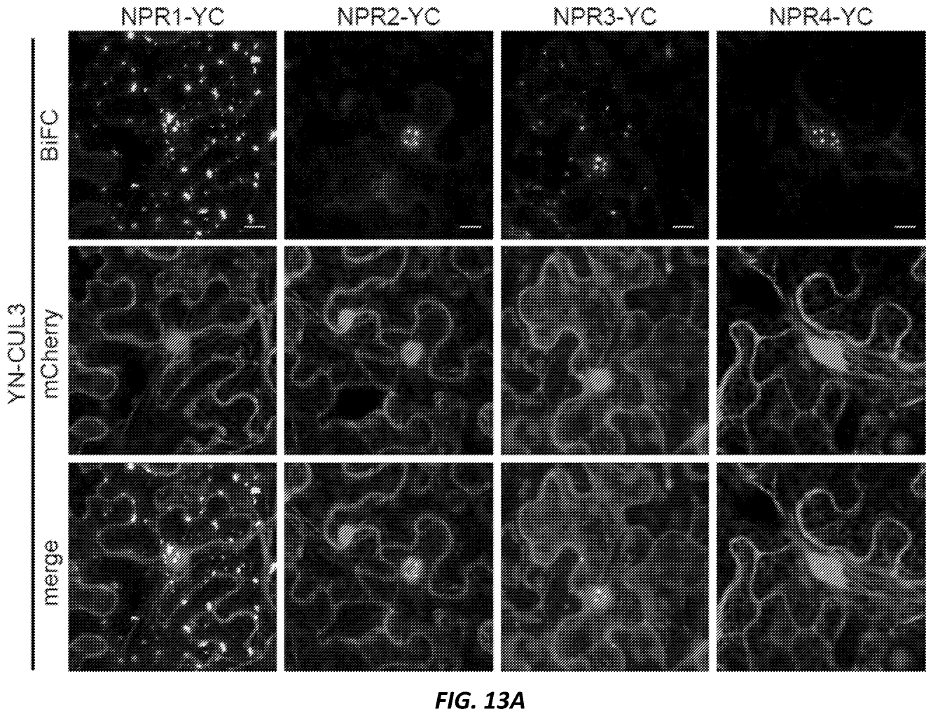

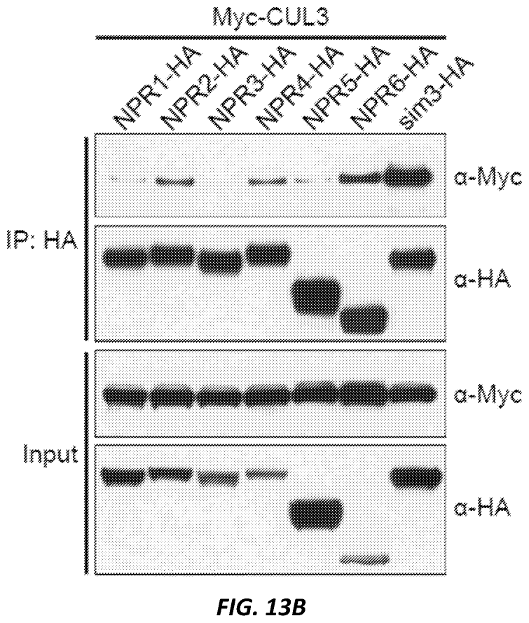

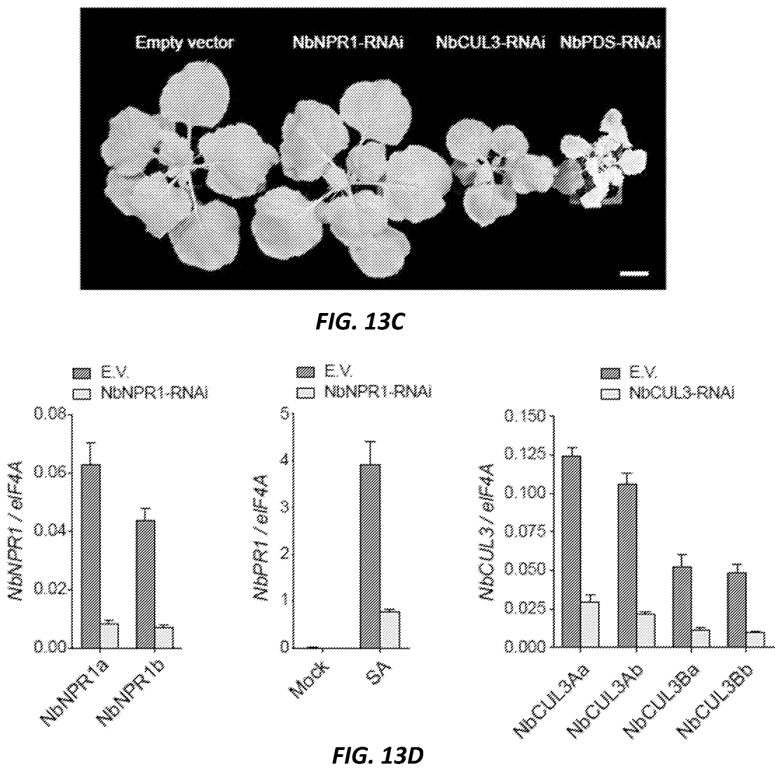

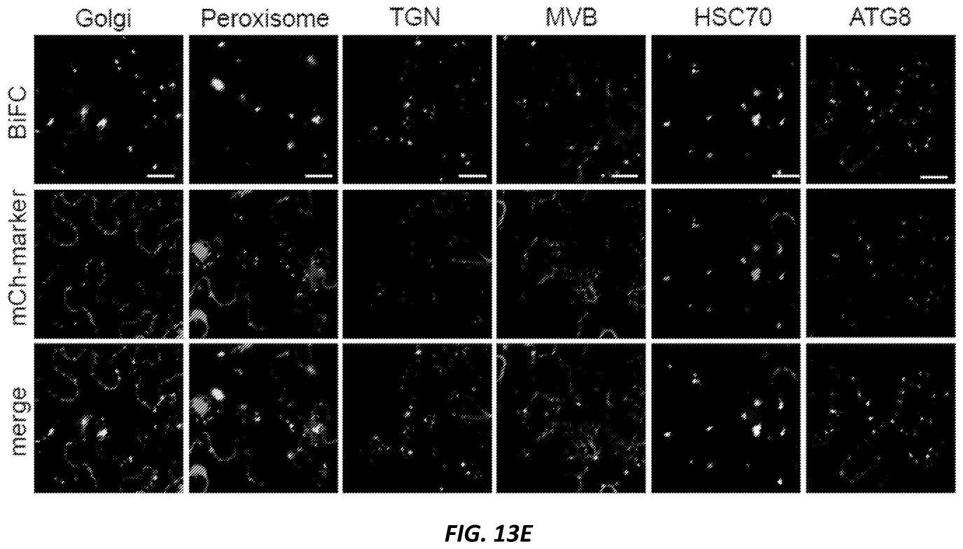

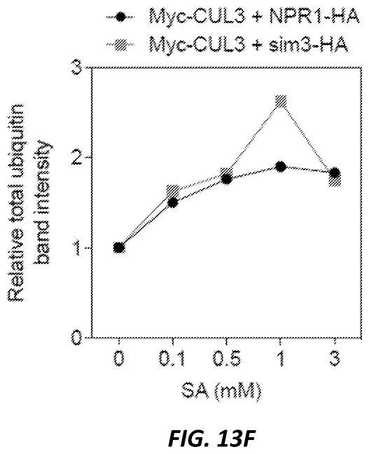

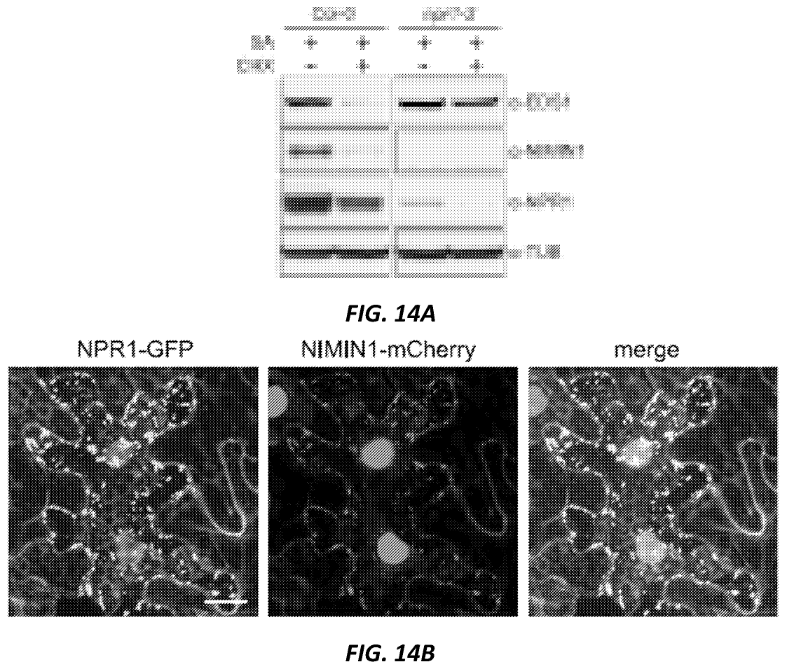

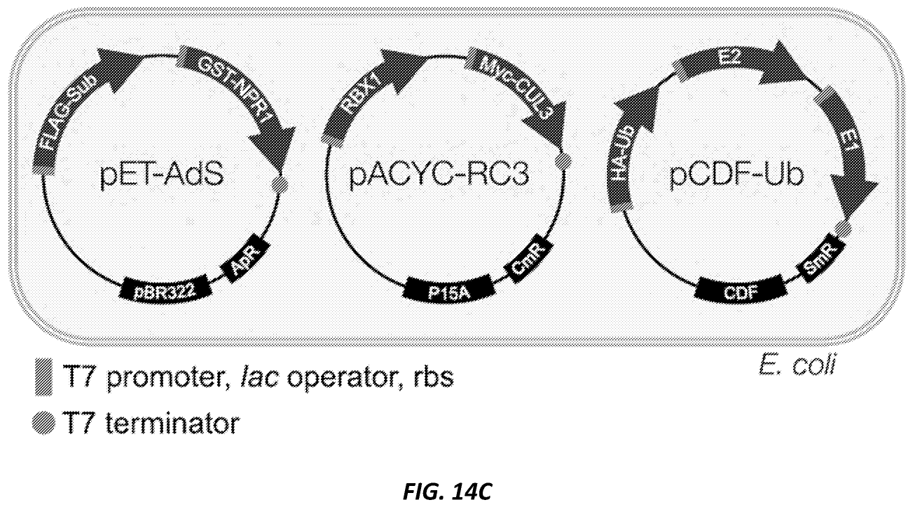

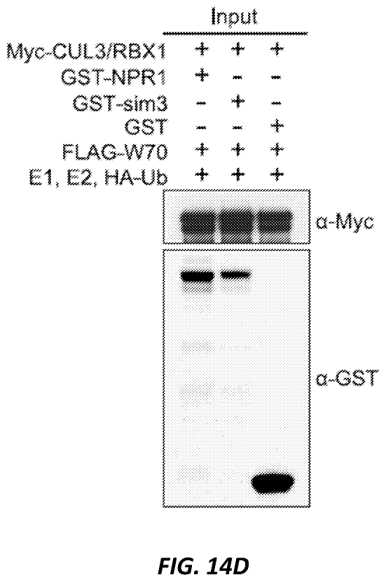

(Avr). At 2 dpi, the adjacent halves were infiltrated with 50 μM estradiol, followed by cell death measurement using the conductivity assay. Data are presented as mean±SD. In FIG. 8B, Col-0 and npr1-2 plants were treated with indicated concentrations of SA (mM) 24 hr before inoculation with Psm ES4326/AvrRpt2. Cell death was measured using the conductivity assay. Data are presented as mean±SD. In FIG. 8C, the ATR1/RPP1 effector/receptor pair was expressed in the NbNPR1-silenced N. benthamiana or E.V. control. At 20 hpi, tissue was sampled and cell death was measured using the conductivity assay in the presence or absence of 1 mM SA (SA or mock, respectively) at 0 and 24 hr after sampling. Data are presented as mean±SD. In FIG. 8D, Col-0 plants were treated with SA for 6 hr, and leaf tissue was used for subcellular fractionation. Proteins from cytoplasmic and nuclear fractions were run on low-resolution gel and probed with α-NPR1 and α-Ub antibodies. In FIG. 8E, the sim3-GFP/npr1-2 transgenic plant was infected at the tip with Psm ES4326/AvrRpt2. At 24 hpi, tissue was sampled from the death-survival boundary (diagram), stained with propidium iodide (PI) to distinguish dead cells (stained nuclei) from living cells (stained apoplast), and imaged. GFP and PI signals were captured from 20 consecutive confocal planes along the Z-axis and across a defined region in the XY-plane covering both dead and living cells (dashed line). Z-projection and XY-stitching were performed to produce the final image. Enlargements from regions adjacent to- and distant from the cell death zone (dashed rectangles) are shown in bottom panels. Scale bar=100 μm (top panels); 20 μm (bottom panels).

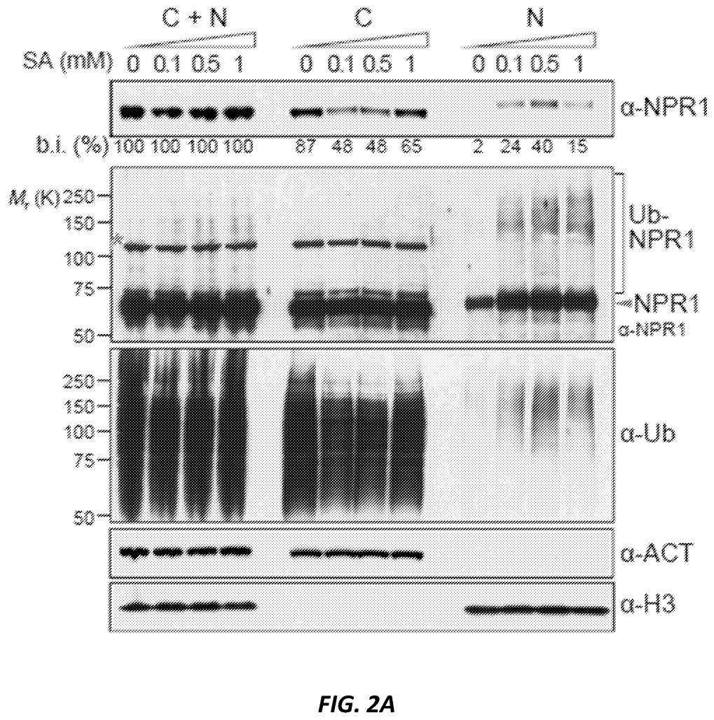

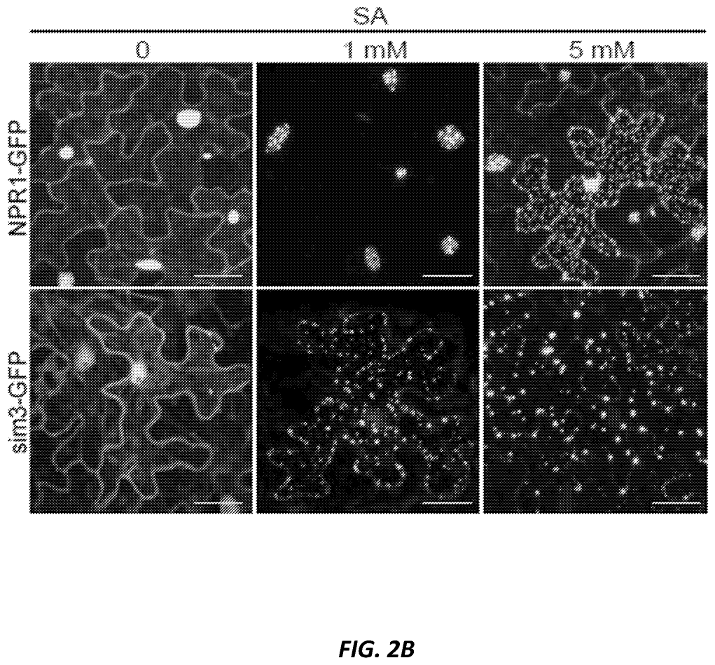

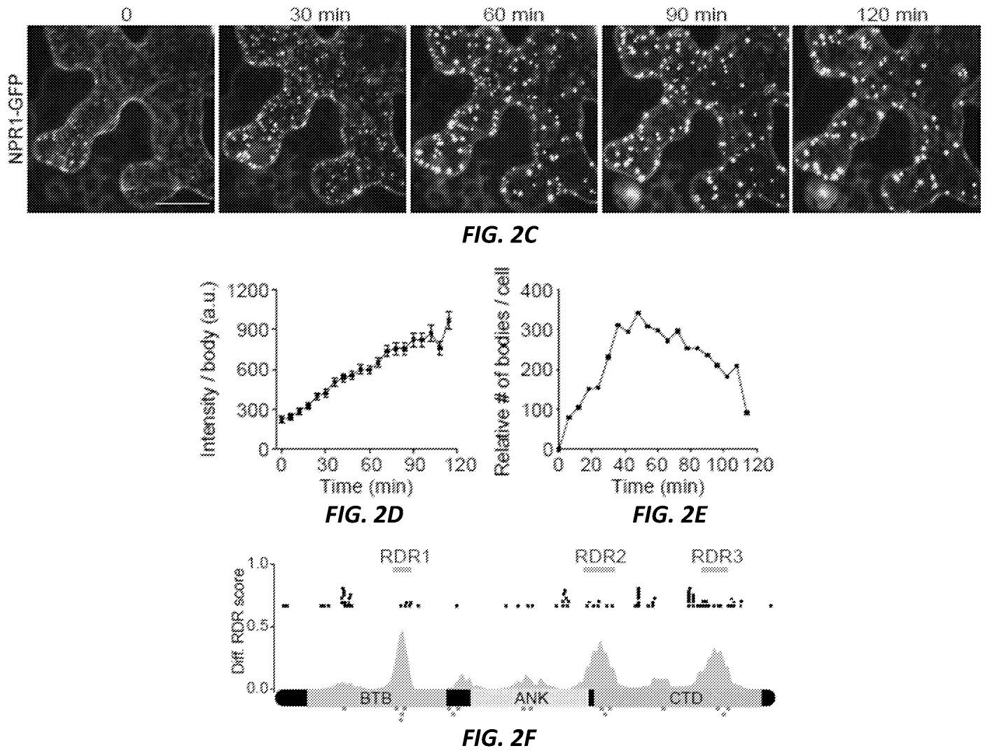

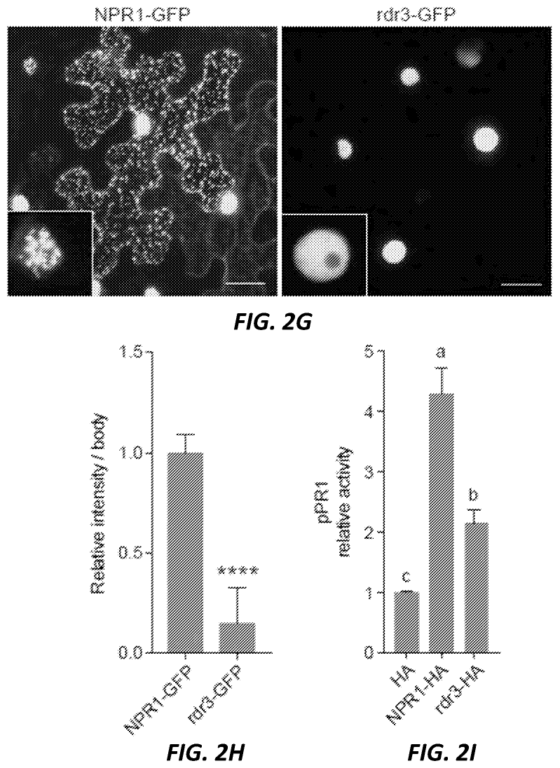

FIGS. 9A-9E show formation of cytoplasmic condensates correlates with predicted redox-sensitive disordered regions in NPR proteins. Related to FIGS. 2A-2I. FIGS. 9A-9C show localization of GFP-fused Arabidopsis NPR1-6 in N. benthamiana after treatment with water (mock) or 5 mM SA for 2 hr (FIG. 9A). Scale bar=10 μm. Insets show enlarged nuclei at lower exposure to visualize the nuclear bodies. FIG. 9B shows quantification of cytoplasmic bodies from the SA-treated samples. Data are presented as mean±SE. FIG. 9C shows validation of NPR-GFP fusion protein expression levels by GFP immunoblotting. FIG. 9D shows interaction of NPR1 with its paralogs NPR2-6 in BiFC assay. Imaging was done after treatment with water (mock) or 1 mM SA for 5 hr. Scale bar=10 μm. FIG. 9E shows prediction of redox-sensitive disorder regions (RDRs) in NPRs (NPR1-6) with the IUPred2a algorithm (iupred2a.elte.hu/). Values represent disorder score predicted for each protein with WT (redox plus) or mutated (redox minus) cysteines. Orange bars indicate the size and position of the identified putative RDRs (shaded regions) with disorder scores>0.5.

FIGS. 10A-10I show analysis of rdr mutants. Related to FIGS. 2A-2I. FIGS. 10A-10C show localization of NPR1-GFP, rdr1-GFP, rdr2-GFP and rdr3-GFP in N. benthamiana in the absence of SA (FIG. 10A). Scale bar=20 μm. FIG. 10B shows quantification of total fluorescence intensity from each body. Values are plotted relative to NPR1-GFP. Data are presented as mean±SE. Validation of fusion protein expression levels by GFP immunoblotting. sim3-GFP is included as a control. FIG. 10D shows nuclear-cytoplasmic partitioning of NPR1-GFP, rdr1-GFP, rdr2-GFP and rdr3-GFP expressed in N. benthamiana in the absence of SA. Total fluorescence intensities from nuclei were quantified as a fraction of total fluorescence intensity of the entire cell. FIG. 10E shows transactivation of the PR1 promoter by NPR1, rdr1-3, sim3 and S55/59D after treatment with 2 mM SA for 24 hr. Values represent the PR1 promoter activity measured as ratio of F-LUC and R-LUC activities and plotted relative to free HA. Data are presented as mean±SD. FIG. 10F shows morphology of npr1-2 mutant and transgenic plants expressing NPR1-GFP, rdr1-GFP, rdr2-GFP and rdr3-GFP in the npr1-2 background. In FIGS. 10G-10I, the npr1-2 mutant and transgenic plants expressing NPR1-GFP/npr1-2, rdr1-GFP/npr1-2 [lines #15 and #18] (FIG. 10G), rdr2-GFP/npr1-2 [lines #8 and #17] (FIG. 10H) and rdr3-GFP/npr1-2 [lines #5 and #8] (FIG. 10D were treated with 1 mM SA 24 hr before inoculation with Psm ES4326/AvrRpt2. Cell death was monitored using the conductivity assay starting at 1 hpi. Data are presented as mean±SD.

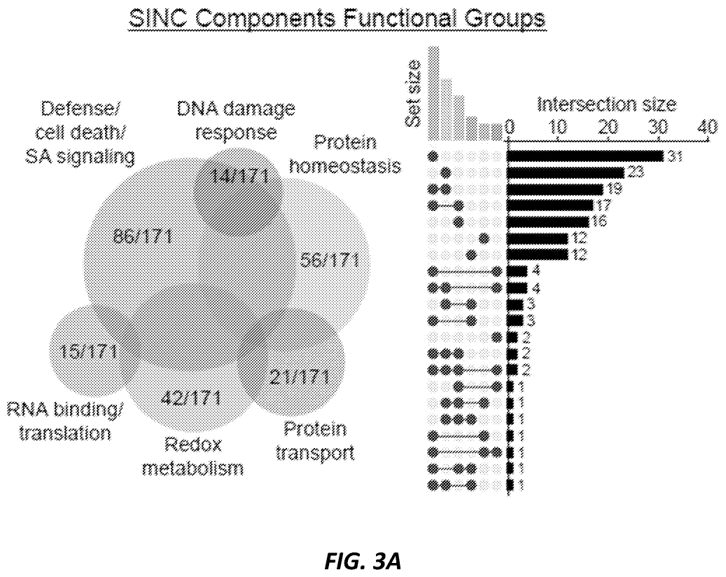

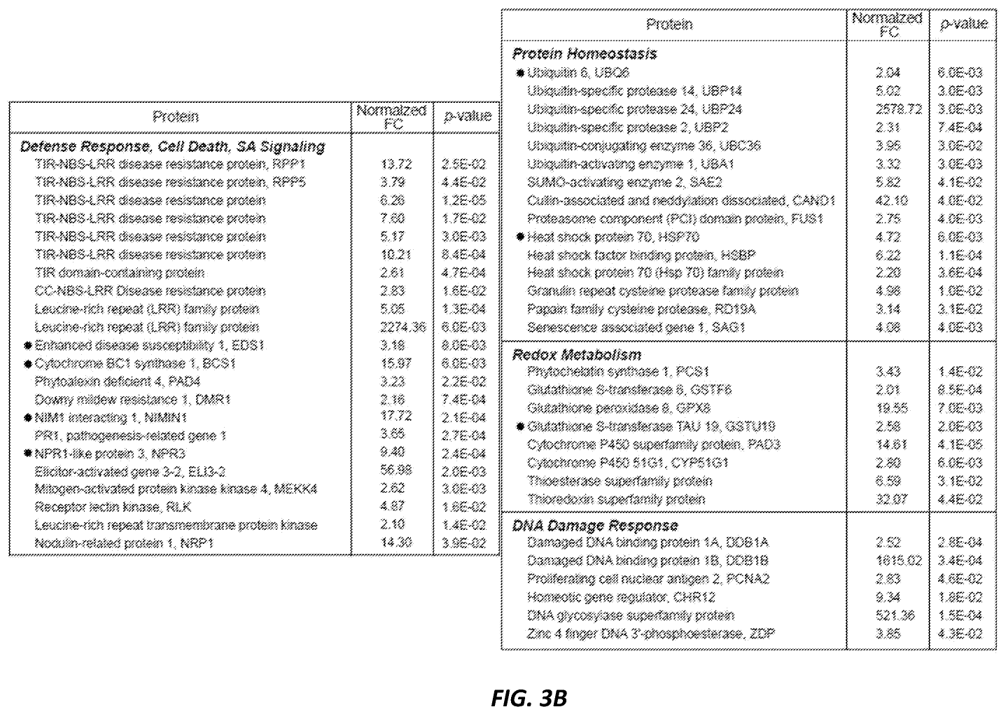

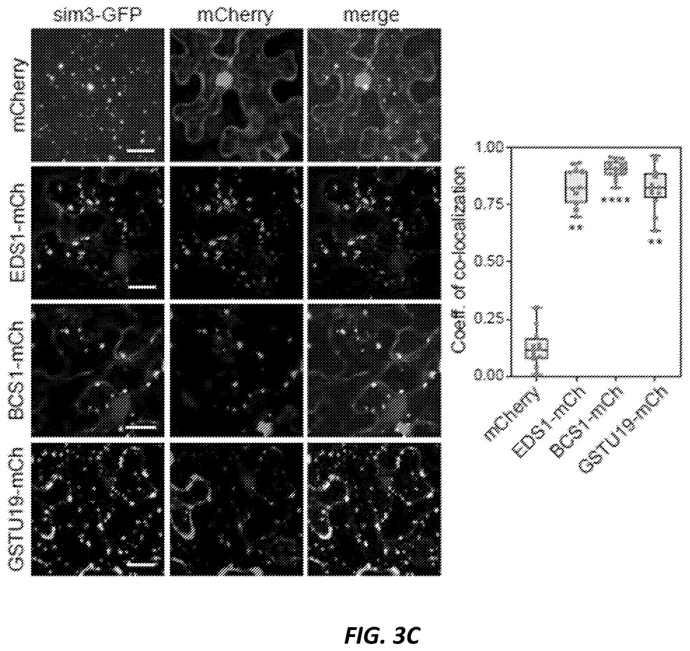

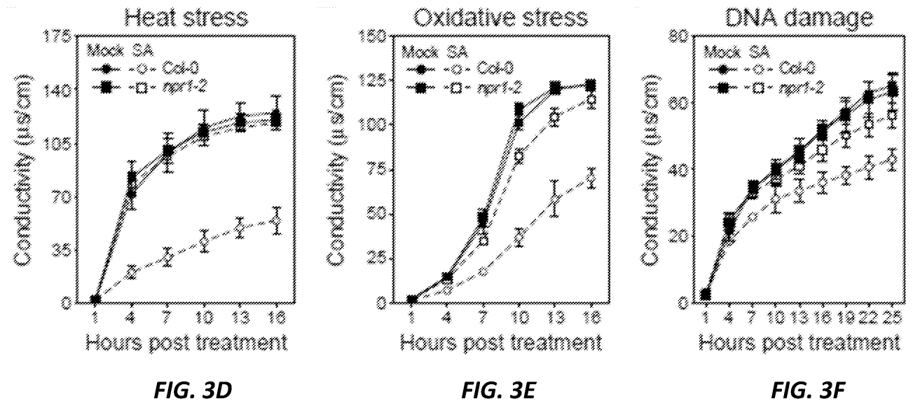

FIGS. 11A-11H show sample preparation, quality test, and GO term analysis of the sim3-GFP interactome. Related to FIGS. 3A-3F. In FIG. 11A, transgenic plants expressing sim3-GFP in WT NPR1 background (sim3-GFP/Col-0) were treated with water (mock) or 1 mM SA for 24 hr, and subcellular localization of sim3-GFP was analyzed. Scale bar=20 μm. In FIG. 11B, total protein was extracted from plants in FIG. 11A, subjected to SDS-PAGE and probed with an α-Ub antibody. Tubulin detected by an α-TUB antibody was used as a loading control. In FIG. 11C, sim3-GFP was immunoprecipitated using GFP-trap beads in three replicate reactions (1, 2, 3) from the extracts in FIG. 11B. A 5 μl aliquot of the beads from each reaction was subjected to SDS-PAGE and silver-stained. The sim3-GFP band (˜94 kDa) is indicated by the red triangle. FIGS. 11D-11E show principal component analysis (PCA) (FIG. 11D) and two-dimensional hierarchical clustering (FIG. 11E) of proteins identified in the sim3-GFP interactome with >2-fold change between SA and mock at p<0.05. For each replicate, values represent intensities detected for individual proteins normalized to the intensity of the bait protein sim3-GFP in the same sample. FIGS. 11F-11H show Gene Ontology (GO) terms of the sim3-GFP interactome. List of proteins (171) identified in the sim3-GFP interactome were submitted to the ShinyGO v0.60: Gene Ontology Enrichment Analysis tool (bioinformatics.sdstate.edu/go/) using default parameters. A Fisher's exact test was applied to identify most enriched GO terms in Biological Process (FIG. 11F), Molecular Function (FIG. 11G), and Cellular Component (FIG. 11H) categories.

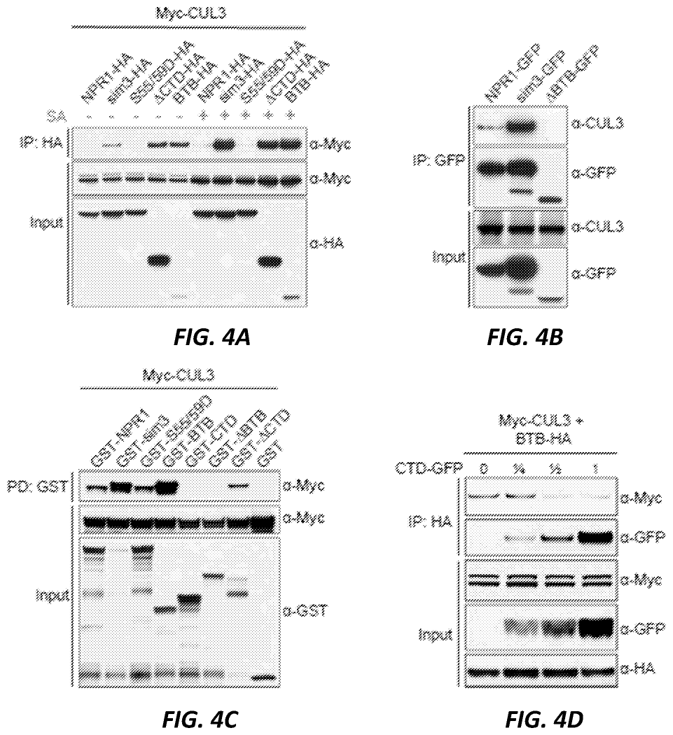

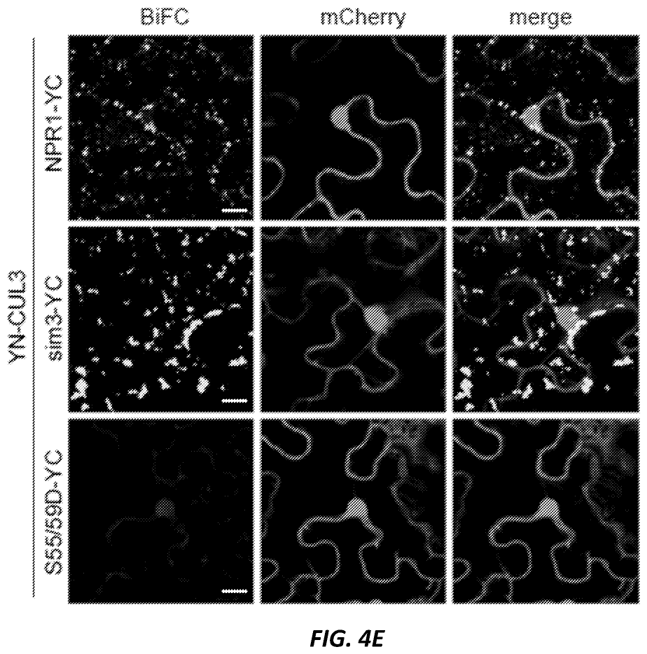

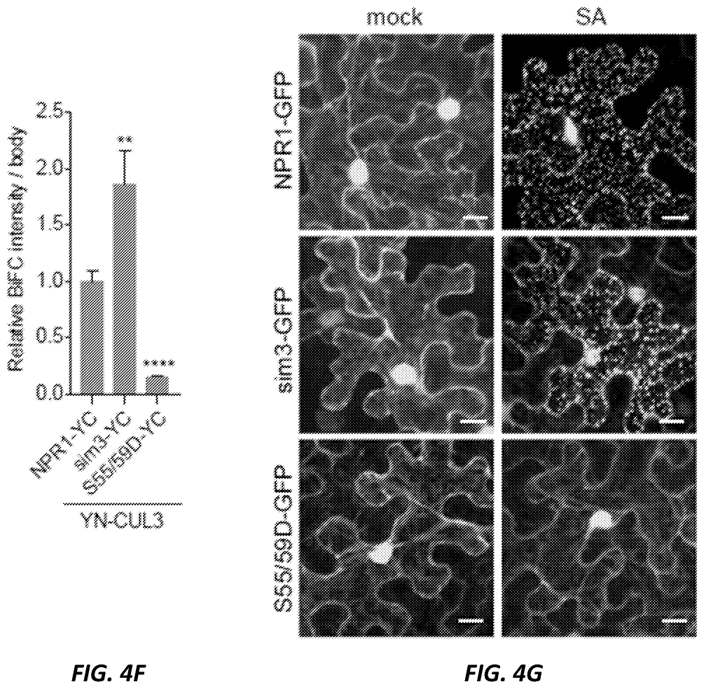

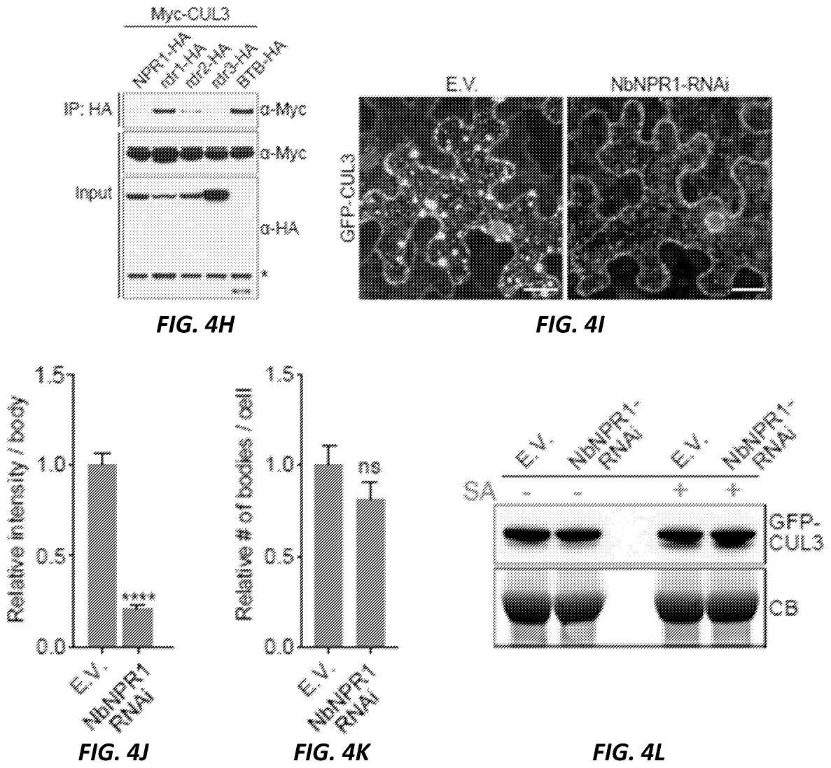

FIGS. 12A-12D show in planta screen of NPR1 mutants for interaction with CUL3. Related to FIGS. 4A-4L. FIG. 12A shows a diagram of NPR1 protein truncations and point mutants used in the screen for interaction with CUL3. FIG. 12B shows representative subcellular localization patterns of GFP-fused WT NPR1, its truncations and point mutants depicted in FIG. 12A after 1 mM SA treatment. Fusion proteins expressed in N. benthamiana followed by treatment with 1 mM SA for 5 hr before imaging. Scale bar=10 μm. FIG. 12C shows interaction between Myc-CUL3 or GFP-CUL3 and HA-tagged NPR1 truncations and point mutant variants. Proteins were expressed in N. benthamiana followed by treatment with 1 mM SA for 5 hr and co-IP with α-HA beads (above). Summary table of co-IP results (below). Letters in the table indicate the predominant subcellular localization for each GFP-fused NPR1 variant: C, cytoplasm; N, nucleus; C/N, cytoplasm and nucleus. Numbers in the table represent interaction strength for each variant quantified using the CUL3 band intensity pulled down by WT NPR1 as 1. FIG. 12D shows localization of ACTD-GFP in N. benthamiana treated with water (mock) or with 1 mM SA for 5 hr before imaging. Scale bar=10 μm.

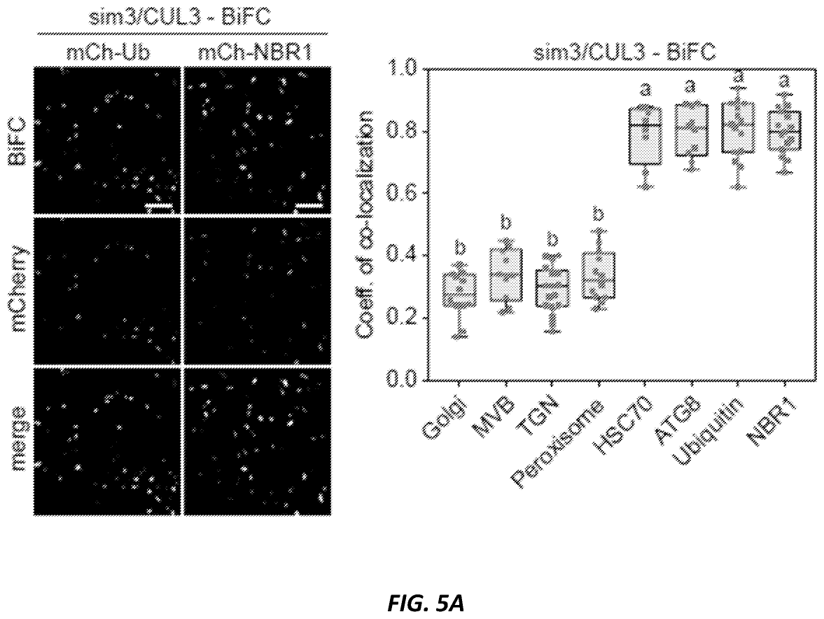

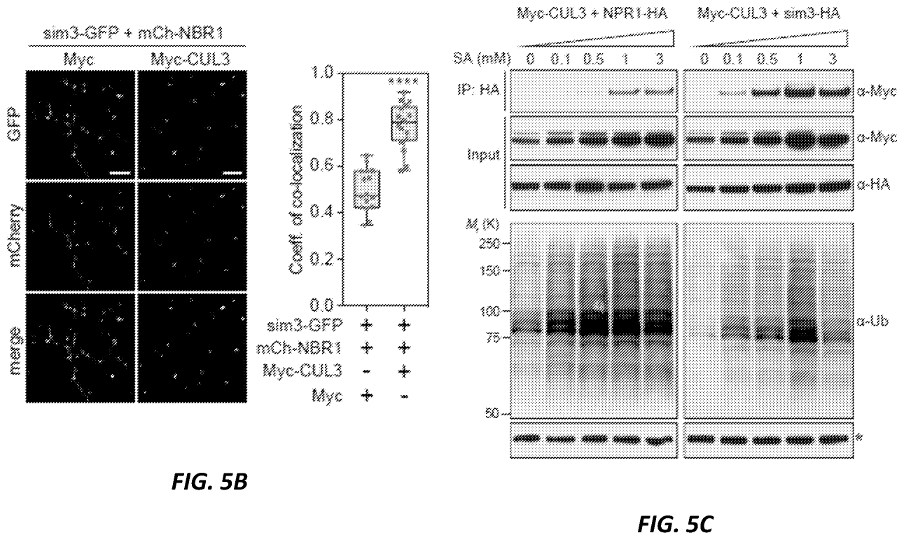

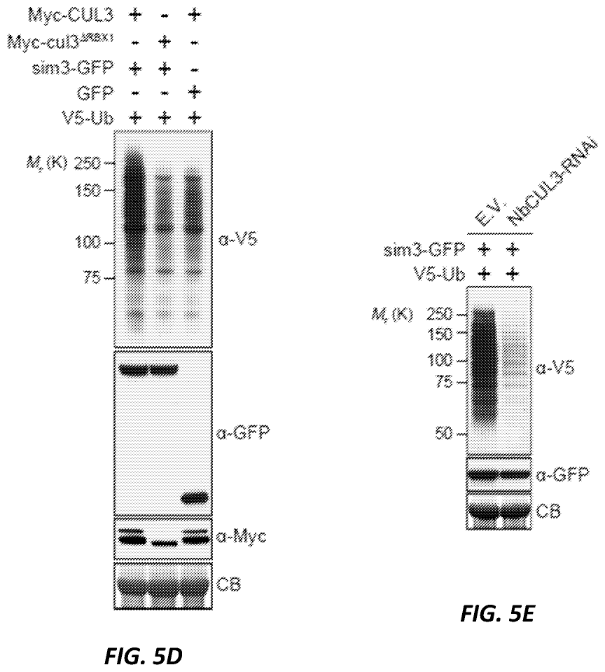

FIGS. 13A-13F show analysis of NPR-CUL3 interactions. Related to FIGS. 4A-4L and 5A-5E. FIG. 13A shows interaction between NPR1 and NPR1-4 in BiFC assay was performed in N. benthamiana. The free mCherry, nYFP-fused CUL3 (YN-CUL3) and cYFP-fused NPR1 (NPR1-YC), NPR2 (NPR2-YC), NPR3 (NPR3-YC) and NPR4 (NPR4-YC) were co-expressed and images were captured from leaves treated with 1 mM SA for 5 hr. Scale bar=10 μm. FIG. 13B shows interaction of Myc-CUL3 with NPR1-HA, NPR2-HA, NPR3-HA, NPR4-HA, NPRS-HA, NPR6-HA or sim3-HA in N. benthamiana. Plants were treated with 1 mM SA for 5 hr followed by co-IP performed on total protein using α-HA beads. FIG. 13C shows images of plants 10 days after inoculation with VIGS (virus-induced gene silencing) vectors carrying the NbNPR1, NbCUL3 or NbPDS (phytoene desaturase) RNAi fragment. Silencing of NbCUL3 caused delay in plant growth, consistent with the essential role of CUL3-dependent ubiquitination. Scale bar=4 cm. FIG. 13D shows validation of gene silencing by qPCR analysis of NbNPR1 (left) and NbPR1 (middle) and NbCUL3 (right) transcripts. Expression of NbPR1 was tested in plants treated with water (mock) or 1 mM SA for 5 hr. Data are presented as mean±SD. FIG. 13E shows representative images of co-localization analysis of sim3/CUL3 BiFC signal with markers of protein bodies and organelles (FIG. 5A). The sim3-YC/YN-CUL3 BiFC pair was co-expressed in N. benthamiana with mCherry-fused markers: TGN (trans-Golgi network/early endosome) and MVB (multivehicular body/late endosome), HSC70 (heat shock protein 70) and ATG8 (autophagy protein 8a). FIG. 13F shows relative band intensity of the total ubiquitination blot shown in the lower panel of FIG. 5C. Intensities were measured along the entire lane and normalized to that of the 0 mM SA sample for each set of interactions.

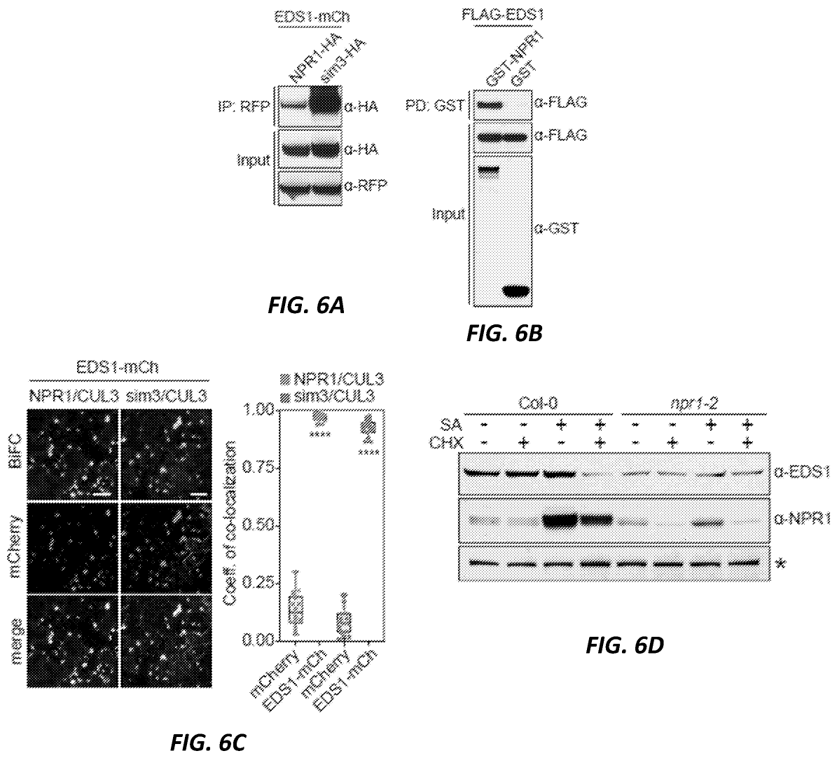

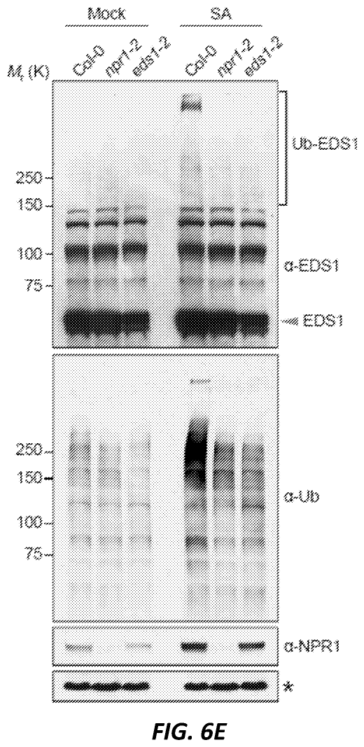

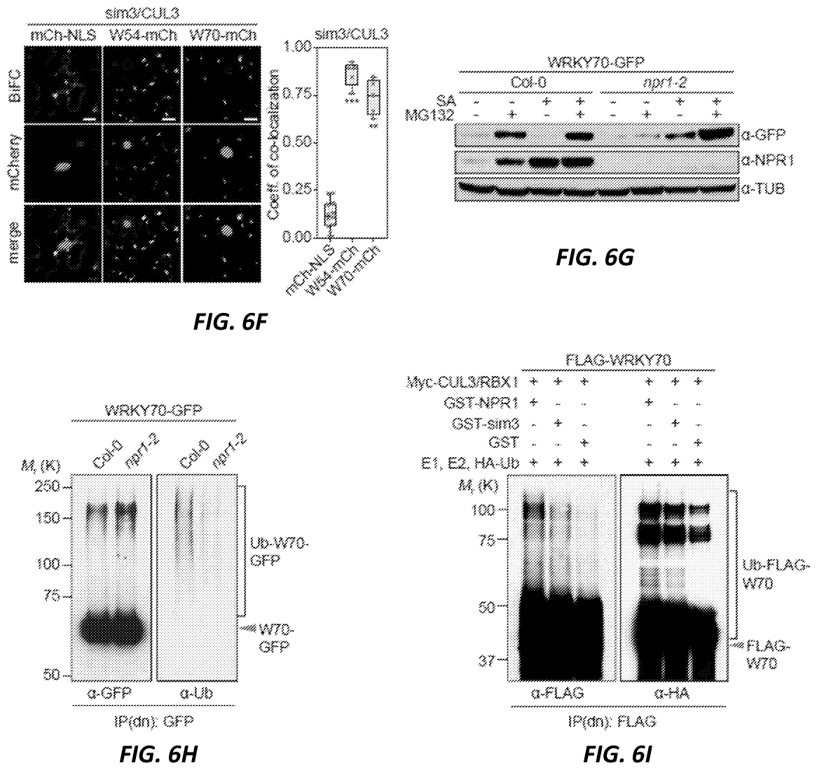

FIGS. 14A-14D show stability and ubiquitination of SINC-localized proteins. Related to FIGS. 6A-6I. FIG. 14A shows stability of EDS1 and NIMIN1 in Col-0 or npr1-2 mutant. Seedlings were treated with water (−) or 1 mM SA for 4 hr, with (+) or without (−) subsequent addition of 100 μM cycloheximide (CHX). After 16 hours of co-incubation, total protein was extracted and immunoblotted with α-EDS1, α-NIMIN1, α-NPR1 and α-TUB (tubulin) antibodies. A higher exposure is shown for theEDS1 blot in the npr1-2 background such that the intensity of control sample (-CHX) is similar to the corresponding sample in the Col-0 background. FIG. 14B shows co-localization of NIM1-interacting 1 (NIMIN1) with NPR1 in SINCs. mCherry-fused NIMIN1 (NINIM1-mCherry) was co-expressed with NPR1-GFP in N. benthamiana followed by treatment with 1 mM SA for 5 hr before imaging. Scale bar=20 μm. FIG. 14C shows a diagram of the E. coli-based ubiquitination system for testing NPR1-dependent ubiquitination of WRKY70. E. coli cells were transformed with three plasmids, each expressing the indicated components of the ubiquitination reaction under the control of lactose operator and Shine-Dalgarno sequence (rbs). Control strains were transformed with the same plasmids except that GST-NPR1 was replaced by either GST-sim3 or GST. FIG. 14D shows an immunoblot of the FLAG-WRKY70 ubiquitination reaction (FIG. 6I) showing levels of Myc-CUL3, GST-NPR1, GST-sim3 and GST before immunoprecipitation.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

A nucleic acid is “operably connected” or “operably linked” when it is placed into a functional relationship with a second nucleic acid sequence. For instance, a promoter is operably linked to a coding sequence if the promoter is connected to the coding sequence or insert site such that it may affect transcription or expression of the coding sequence.

The nucleic acids described herein be operably linked to one or more expression control elements. “Expression control elements” are sequences that modulate expression of the gene, either through modulation of transcription of the gene, modulation of translation on an mRNA transcribed from the gene, or a combination of modulation of transcription and translation. Expression control elements include, but are not limited to, promoters, enhancers, 3′ untranslated sequence, and 5′ untranslated sequences.

The nucleic acids described herein may be operably linked to a promoter or a combination of a promoter and one or more uORFs. The terms “promoter,” “heterologous promoter,” “promoter region,” or “promoter sequence” refer generally to transcriptional regulatory regions of a gene, which may be found at the 5′ or 3′ end of the coding region, or within the coding region of the heterologous coding sequence, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of RNA polymerase. The promoter may be the endogenous promoter of an endogenous gene modified to include heterologous R-motif, uORF, and/or 5′ regulatory sequences (i.e., separately or in combination). The promoter may be natively associated with the 5′ UTR chosen, but be operably connected to a heterologous coding sequence.

Promoters include, but are not limited to, constitutive promoters, inducible promoters, temporally-regulated promoters, developmentally regulated promoters, chemically regulated promoters, tissue-preferred promoters, and tissue-specific promoters. A promoter suitable for expression in plants includes, but is not limited to, a TBF1 promoter (from any plant species including Arabidopsis), a 35S promoter (such as from a cauliflower mosaic virus), a ubiquitin promoter, a tCUP cryptic constitutive promoter, a Rsyn7 promoter, a pathogen-inducible promoter, a maize In2-2 promoter, a tobacco PR-1a promoter, a glucocorticoid-inducible promoter, an estrogen-inducible promoter, a tetracycline-inducible promoter, a tetracycline-repressible promoter, a T3 promoter, a T7 promoter, and a SP6 promoter. Those of skill in the art are familiar with a wide variety of additional promoters for use in various cell types. In some embodiments, the promoter includes a plant promoter. In some embodiments, the promoter includes a plant promoter inducible by a plant pathogen or chemical inducer. The promoter may be a seed-specific or fruit-specific promoter.

An upstream open reading frame (uORF) is an open reading frame (ORF) within the 5′ untranslated region (5′ UTR) of an mRNA. uORFs can regulate eukaryotic gene expression, such as through suppression of translation. Translation of the uORF may inhibit or increase downstream expression of the primary ORF (e.g., by translation suppression). As one example, a uORF can be a TBF1 uORF (e.g., Arabidopsis thaliana TBF1), such as uORF1 or uORF2. See, e.g., Pajerowska-Mukhtar et al. (2012) Curr. Biol. 22 (2): 103-112; Xu et al. (2017) Nature 545(7655):491-494; US 2018-0273965; U.S. Pat. No. 10,584,346; US 2015-0113685; U.S. Pat. No. 10,017,773; WO 2013/096567; US 2019-0352664; and WO 2018/144831, each of which is herein incorporated by reference in its entirety for all purposes.

“Salicylic acid-induced NPR1 condensates” (SINCs) are cytoplasmic organelle-like structures that contain NPR1 protein and Cullin 3 RING E3 ligase (CLR3) and are enriched in proteins that regulate death, redox metabolism, DNA damage response, and protein quality control machineries, including ubiquitination. SINC formation in wild-type plants is dependent on NPR1 and salicylic acid. In the absence of salicylic acid, wild-type NPR1 does not induce formation of SINCs. However, in the presence or salicylic acid, wild-type NPR1 initiates formation of condensates.

“Salicylic acid-independent NPR1 condensates” are NPR1 condensates that are functionally similar to SINCs and form in the absence of salicylic acid. Formation of salicylic acid-independent NPR1 condensates is induced by certain mutant npr1 proteins described herein in the absence of salicylic acid.

An “intrinsically disordered region” (IDR) is a region of a protein that does not have, or is not predicted to have, a fixed or ordered three-dimensional structure in the absence of interaction partners. IDRs can be determined or predicted using methods known in the art. IDRs can be predicted using algorithms such as, but not limited to, the IUPred2a algorithm. IUPred2A can be used to predict disordered protein regions using the IUPred2 algorithm and optionally disordered binding regions using ANCHOR2. IUPred2 returns a score between 0 and 1 for each residue in an input protein (amino acid) sequence corresponding to the probability that the given residue is part of a disordered region. IUPred2A is also capable of identifying protein regions that do or do not adopt a stable structure depending on the redox state of their environment.

A “redox-sensitive intrinsically disordered region” (RDR) is an IDR that is sensitive to oxidations. Redox-sensitivity can be determined or predicted using methods known in the art. Redox-sensitivity can be predicted using algorithms such as, but not limited to, IUPred2a algorithm. RDRs can be determined or predicted using methods known in the art. RDRs can be predicted using algorithms such as, but not limited to, the IUPred2a algorithm. In some embodiments, an RDR contains one or more cysteine residues. In some embodiments, an RDR region comprises a string of 5 or more contiguous amino acids wherein the differential IDR score ((Redox minus)−(Redox plus)) determined for each amino acid is greater than or equal to about 0.15. Redox minus and redox plus scores can be determined using the IUPred2a algorithm. In some embodiments, an RDR region comprises a string of 5, 6, 7, 8, 9, 10, 11, 12, 13, 14, 15, 16, 17, 18, 19, 20, or more consecutive amino acids wherein the differential IDR score ((Redox minus)−(Redox plus)) each amino acid is greater than or equal to about 0.15.

A “homolog” or “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identity to the known reference sequence. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences. Homologous sequences can include, for example, orthologs (orthologous sequences) and paralogs (paralogous sequences). Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

“NPR1” (non-expresser of pathogenesis related (PR) genes 1) is a plant gene that encodes the NPR1 protein. NPR1 is a positive regulator of systemic acquired resistance. NPR1 contains a Broad-Complex, Tramtrack and Bric a brac (BTB) domain, and three ankyrin-repeat domains.

Unless otherwise defined, all technical terms used herein have the same meaning as commonly understood by one of ordinary skill in the art to which this disclosure belongs.

Treating plants with salicylic acid (SA) can protect them from cell death induced by both biotic and abiotic stresses, such as a broad spectrum of pathogens, high and low temperature, oxidative stress, and DNA damage through the formation of NPR1 condensates. These SA-induced NPR1 condensates (SINCs) contain many plant nucleotide-binding and leucine-rich repeat (NB-LRR) immune receptors and signaling components, such as EDS1 and PAD4, as well as components in the heat response, redox regulation, DNA damage repair and protein homeostasis (ubiquitination/degradation) pathways. SINC formation serves as a sink for maintaining protein homeostasis during a diverse array of stresses and make plants more resistant. Since SINC formation is an intrinsic property of NPR1, it can be used as a target for engineering broad spectrum stress resistance in different organisms because many of the SINC proteins and the processes that they represent are highly conserved in evolution.

The present disclosure provides, is based, in part on the discovery by the inventors that NPR1 promotes cell survival by targeting substrates for ubiquitination and degradation through salicylic acid (SA)-driven phase separation into cytoplasmic condensates. Further, the inventors show that NPR1 condensates are enriched in cell death regulators including nucleotide-binding leucine-rich repeat immune receptors, redox metabolism proteins, DNA damage repair and protein quality control machineries. Phase separation of NPR1 is required for recruitment of the Cullin 3 RING E3 ligase complex to the condensates and NPR1 can promote cell survival by degrading EDS1 and specific WRKY transcription factors required for ETI. The discovery of distinct functional groups of proteins in the SA-induced NPR1 condensates, herein termed “SINCs”, suggests that NPR1 modulates survival by converging multiple stress-responsive processes in this quasi organelle.

As described above and further described below in the examples, in response to SA, wild-type NPR1 can be found in cytoplasmic condensates (SINCs) that correlate with decreased plant cell death and increased plant tolerance to various stresses, including biotic and abiotic stress. Described herein are variant NPR1 proteins (npr1 proteins) that spontaneously or constitutively form cytoplasmic condensates (NPR1 condensates), i.e., in the absence of SA. The described npr1 proteins induce formation of NPR1 condensates at greater frequency compared to wild type NPR1 protein in the absence of SA. These salicylic acid (SA)-independent NPR1 condensates also correlate with decreased plant cell death and increased plant tolerance to various stresses, including biotic and abiotic stress. Expression of a npr1 protein in a plant cell or plant can be used to increase plant stress tolerance and reduce plant cell death in response to stress.

### NPR1 Proteins

In some embodiments, a npr1 protein comprises a NPR1 protein having one or more mutations in at least one redox-sensitive intrinsically disordered region (RDR), wherein the one or more mutations result in the npr1 protein forming salicylic acid-independent NPR1 condensates. In some embodiments, the npr1 protein retains the cytoplasmic functions of NPR1. In some embodiments, the npr1 protein retains the nuclear functions of NPR1. In some embodiments, the npr1 protein retains both the cytoplasmic and nuclear functions of NPR1. Formation of salicylic acid-independent NPR1 condensates is readily determined using the methods described herein. NPR1 contains three RDRs (RDR1, RDR2, and RDR3). The three RDRs of the A. thaliana NPR1 are located at amino acids 140-160, 368-404, and 510-539 (shown in boxes) of

SEQ ID NO: 1
(MDTTIPGFADSYE1SSTSFVATDNTDSSIVYLAAEQ
VLTGPDVSALQLLSNSFESVFDSPDDFYSDAKLVLSDGREVSFHRCVLSARSSFFKSA

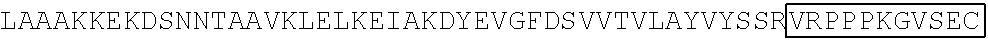

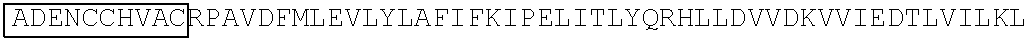

ANICGKACMKLLDRCKEIIVKSNVDMVSLEKSLPEELVKEIIDRRKELGLEVPKVKKH
VSNVHKALDSDDIELVKLLLKEDHTNLDDACALHFAVAYCNVKTATDLLKLDLAD

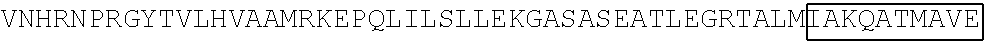

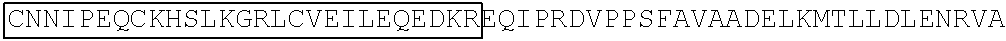

LAQRLFPTEAQAAMEIAEMKGTCEFIVTSLEPDRLTGTKRTSPGVKIAPFRILEEHQSR

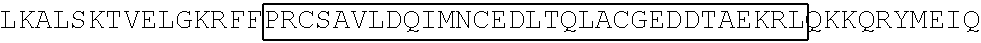

ETLKKAFSEDNLELGNSSLTDSTSSTSKSTGGKRSNRKLSHRRR).

The RDR regions of homologs and/or orthologs of A. thaliana NPR1 can be identified using RDR predicting algorithms as is described for A. thaliana NPR1. Alternatively, RDR regions of homologs and/or orthologs of A. thaliana NPR1 can be identified by identifying the regions of the NPR1 homologs and/or orthologs corresponding to amino acids 140-160, 368-404, and 510-539 of SEQ ID NO: 1. Suitable orthologs of A. thaliana NPR1 include, but are not limited to, the NPR1 of tobacco, tomato, grape, barley, rice, soybean, melon, corn, rapeseed, cabbage, broccoli, radish, and mustard. Orthologs of SEQ ID NO: 1 include, but are not limited to: SEQ ID NOS: 2-12 and 30-34. The RDR2 region of A. thaliana NPR1 corresponds to amino acids 368-404 or SEQ ID NO: 1. The corresponding RDR2 regions of SEQ ID NOS. 2-12 and 30-34 are shown in Table 1. The corresponding RDR1 and RDR3 regions of SEQ ID NOS. 2-12 and 30-34 can be similarly determined by homology alignment with SEQ ID NO: 1.

TABLE 1
NPR1 RDR2 sequence alignment: NPR1 RDR2 sequence in Arabidopsis
thaliana and orthologs.

| | | SEQ |
|---|---|---|
| Plant | RDR2 Sequence Alignment | ID NO. |
| Arabidopsis thaliana | RTALMIAKQATMAVECNNIPEQCKHSLKGRLCVEILEQEDKR | 13 |
| Nicotiana benthamiana | RKALQIAKRLTRLVDFSKSPEEGKSASKDRLCIEILEQAERR | 14 |
| Solanum Lycopersicon | KKALQIAKRLTRLVDFTKSTEEGKSAPKDRLCIEILEQAERR | 15 |
| Vitis vinifera | RNALQIAKRLTRAVDYHKSTEEGKPSPKDQLCVEVLEQAERR | 16 |
| Hordeum vulgare | RKAVQIAKRLTKHGDYFGNTEEGKPSPNDKLCIEILEEAERR | 17 |
| Medicago truncatula | RKALQISKRCTKAVDYYKSTEEGKVSSNDRLCIEILEQAERR | 18 |
| Nicotiana tabacum | RKALQIAKRLTRLVDFSKSPEEGKSASNDRLCIEILEQAERR | 19 |
| Oryza sativa | RKAVQISKRLTKQGDYFGVTEEGKPSPKDRLCIEILEQAERR | 20 |
| Glycine max | RKALQISKRLTKAVDYYKSTEEGKVSCSDRLCIEILEQAERR | 21 |
| Populus trichocarpa | RKALQISKRLTRAMDYHKSTEEGKASPKERLCIEILEQAERR | 22 |
| Cucumis melo | RTAANICQRLTRPKDYHAKTEKGQETNKDRLCIDILEREMWR | 23 |
| Zea mays | RKAVQISKRLTKHGDYFGPTEDGKPSPKDRLCIEVLEQAERR | 24 |
| Raphanus sativus | RTALVIAKQVTKATECCIL ERGKLSAKGGVCVEILKEPDNK | 25 |
| Brassica napus | RTALLIAKQVTKAAECCIL EKGKLAAKGGVCVEILKQPDNK | 26 |
| Brassica oleracea | RTALLIAKQVTKAAECCIL EKGKLAAKGGVCVEILKQPDNT | 27 |
| Brassica rapa | RTALLIAKQVTKAAECCIL EKGKLAAKGGVCVEILKQPDNK | 28 |
| Brassica juncea | RTALLIAKQVTKAAECCIL EKGKLAAKGGVCVEILKQPDNK | 29 |

In some embodiments, a npr1 protein comprises an A. thaliana NPR1 protein having one or more mutations in at least one redox-sensitive intrinsically disordered region (RDR), wherein the one or more mutations result in the npr1 protein forming salicylic acid-independent NPR1 condensates. In some embodiments, a npr1 protein comprises an ortholog of an A. thaliana NPR1 protein having one or more mutations in at least one redox-sensitive intrinsically disordered region (RDR), wherein the one or more mutations result in the npr1 protein forming salicylic acid-independent NPR1 condensates. Overexpression of A. thaliana NPR1 or its orthologs has been shown to enhance resistance in a number of horticultural crop plants, including grape, carrot, tomato, apple, citrus, tobacco, and strawberry and in high-acreage agronomic crops such as rice, wheat, soybean, peanut, and potato. See, e.g., Silva et al. (2018) Hortic. Res. 5:15, herein incorporated by reference in its entirety for all purposes. The ortholog can be, but is not limited to, a Nicotiana benthamiana NPR1, a Solanum lycopersicon NPR1, a Vitis vinifera NPR1, a Hordeum vulgare NPR1, a Medicago truncatula NPR1, a Nicotiana tabacum NPR1, a Oryza sativa NPR1, a Glycine max NPR1, a Populus trichocarpa NPR1, a Cucumis melo NPR1, a Zea mays NPR1, a Raphanus sativus NPR1, a Brassica napus NPR1, a Brassica oleracea NPR1, a Brassica rapa NPR1, a Brassica juncea NPR1. The ortholog can be, but is not limited to, a protein comprising the amino acid sequence of SEQ ID NO: 2-12 and 30-34.

In some embodiments, the one or more mutations in the at least one RDR reduce the redox-sensitivity of the RDR.

In some embodiments, a npr1 protein comprises a NPR1 protein having mutations of one or more cysteines in at least one RDR. The one or more cysteines can be located in a single RDR, 2 RDRs (e.g., RDR1 and RDR2, RDR2, and RDR3, or RDR1 and RDR3), 3 RDRs (e.g., RDR1, RDR1 and RDR3), or a combination thereof. In some embodiments, a npr1 protein comprises an A. thaliana NPR1 protein having mutations of one or more cysteines in at least one RDR. The one or more cysteines can be located in a single RDR, 2 RDRs (e.g., RDR1 and RDR2, RDR2, and RDR3, or RDR1 and RDR3), 3 RDRs (e.g., RDR1, RDR1 and RDR3), or a combination thereof. In some embodiments, a npr1 protein comprises an ortholog of an A. thaliana NPR1 protein having mutations of one or more cysteines in at least one RDR. The ortholog can be, but is not limited to, a Nicotiana benthamiana NPR1, a Solanum lycopersicon NPR1, a Vitis vinifera NPR1, a Hordeum vulgare NPR1, a Medicago truncatula NPR1, a Nicotiana tabacum NPR1, a Oryza sativa NPR1, a Glycine max NPR1, a Populus trichocarpa NPR1, a Cucumis melo NPR1, a Zea mays NPR1, a Raphanus sativus NPR1, a Brassica napus NPR1, a Brassica oleracea NPR1, a Brassica rapa NPR1, a Brassica juncea NPR1. The ortholog can be, but is not limited to, a protein comprising the amino acid sequence of SEQ ID NO: 2-12 and 30-34. The npr1 protein can have a mutation of a single cysteine, 2 cysteines, 3 cysteines, 4 cysteines, 5 cysteines, 6 cysteines, or 7 or more cysteines. The mutation can be a substitution, a deletion, or a combination thereof. The mutation at each position can independently be an alanine substitution, a glycine substitution, a serine substitution, a threonine substitution, or a deletion. In some embodiments, the substitution is an alanine.

In some embodiments, a npr1 protein comprises a NPR1 protein having mutations of one or more cysteines, wherein the cysteines are located in a region corresponding to residues 140-160, 368-404, and/or 510-539 of SEQ ID NO: 1. In some embodiments, a npr1 protein comprises an A. thaliana NPR1 protein having mutations of one or more cysteines, wherein the cysteines are located in a region corresponding to residues 140-160, 368-404, and/or 510-539 of SEQ ID NO: 1. In some embodiments, a npr1 protein comprises an ortholog of an A. thaliana NPR1 protein having mutations of one or more cysteines, wherein the cysteines are located in a region corresponding to residues 140-160, 368-404, and/or 510-539 of SEQ ID NO: 1. The ortholog can be, but is not limited to, a Nicotiana benthamiana NPR1, a Solanum lycopersicon NPR1, a Vitis vinifera NPR1, a Hordeum vulgare NPR1, a Medicago truncatula NPR1, a Nicotiana tabacum NPR1, a Oryza sativa NPR1, a Glycine max NPR1, a Populus trichocarpa NPR1, a Cucumis melo NPR1, a Zea mays NPR1, a Raphanus sativus NPR1, a Brassica napus NPR1, a Brassica oleracea NPR1, a Brassica rapa NPR1, a Brassica juncea NPR1. The ortholog can be, but is not limited to, a protein comprising the amino acid sequence of SEQ ID NO: 2-12 and 30-34. The npr1 protein can have a mutation of a single cysteine, 2 cysteines, 3 cysteines, 4 cysteines, 5 cysteines, 6 cysteines, or 7 or more cysteines. The mutation can be a substitution, a deletion, or a combination thereof. The mutation at each position can independently be an alanine substitution, a glycine substitution, a serine substitution, a threonine substitution, or a deletion. In some embodiments, the substitution is an alanine. The cysteine residues in an ortholog of A. thaliana NPR1 may not be in the exact same numerical positions as the cysteines in A. thaliana NPR1. For example, the cysteine residue at position 394 of SEQ ID NO: 1 occurs at position 396 of the Zea mays ortholog (SEQ ID NO: 12).

In some embodiments, a npr1 protein comprises a NPR1 protein having mutations of one or more cysteines, wherein the cysteines are located in a region corresponding to residues 368-404 of SEQ ID NO: 1. In some embodiments, a npr1 protein comprises an A. thaliana NPR1 protein having mutations of one or more cysteines, wherein the cysteines are located in a region corresponding to residues 368-404 of SEQ ID NO: 1. In some embodiments, a npr1 protein comprises an ortholog of an A. thaliana NPR1 protein having mutations of one or more cysteines, wherein the cysteines are located in a region corresponding to residues 368-404 of SEQ ID NO: 1. The ortholog can be, but is not limited to, a Nicotiana benthamiana NPR1, a Solanum lycopersicon NPR1, a Vitis vinifera NPR1, a Hordeum vulgare NPR1, a Medicago truncatula NPR1, a Nicotiana tabacum NPR1, a Oryza sativa NPR1, a Glycine max NPR1, a Populus trichocarpa NPR1, a Cucumis melo NPR1, a Zea mays NPR1, a Raphanus sativus NPR1, a Brassica napus NPR1, a Brassica oleracea NPR1, a Brassica rapa NPR1, a Brassica juncea NPR1. The ortholog can be, but is not limited to, a protein comprising the amino acid sequence of SEQ ID NO: 2-12 and 30-34. The npr1 protein can have a mutation of a single cysteine, 2 cysteines, 3 cysteines, 4 cysteines, 5 cysteines, 6 cysteines, or 7 or more cysteines. The mutation can be a substitution, a deletion, or a combination thereof. The mutation at each position can independently be an alanine substitution, a glycine substitution, a serine substitution, a threonine substitution, or a deletion. In some embodiments, the substitution is an alanine.

In some embodiments, a npr1 protein comprises a NPR1 protein having a mutation of a cysteine located at a position corresponding to residue 394 of SEQ ID NO: 1. In some embodiments, a npr1 protein comprises an A. thaliana NPR1 protein having a mutation of a cysteine at position 394 of SEQ ID NO: 1. In some embodiments, a npr1 protein comprises an ortholog of an A. thaliana NPR1 protein having a mutation of a cysteine located at a position corresponding to residue 394 of SEQ ID NO: 1. The ortholog can be, but is not limited to, a Nicotiana benthamiana NPR1, a Solanum lycopersicon NPR1, a Vitis vinifera NPR1, a Hordeum vulgare NPR1, a Medicago truncatula NPR1, a Nicotiana tabacum NPR1, a Oryza sativa NPR1, a Glycine max NPR1, a Populus trichocarpa NPR1, a Cucumis melo NPR1, a Zea mays NPR1, a Raphanus sativus NPR1, a Brassica napus NPR1, a Brassica oleracea NPR1, a Brassica rapa NPR1, a Brassica juncea NPR1. The ortholog can be, but is not limited to, a protein comprising the amino acid sequence of SEQ ID NO: 2-12 and 30-34. The mutation can be a substitution or a deletion. The substitution can be an alanine substitution, a glycine substitution, a serine substitution, or a threonine substitution. In some embodiments, the substitution is an alanine. In some embodiments, the npr1 protein comprises the amino acid sequence of SEQ ID NO: 137, 138, 139, 140, 141, 142, 143, 144, 145, 146, 147, 148, 149, 150, 151, 152, or 153.

In some embodiments, a npr1 protein comprises an A. thaliana NPR1 protein having a mutation of a cysteine at position 378 of SEQ ID NO: 1, position 385 of SEQ ID NO: 1, position 394 of SEQ ID NO: 1, positions 378 and 385 of SEQ ID NO: 1, positions 378 and 394 of SEQ ID NO: 1, positions 385 and 394 of SEQ ID NO: 1, or positions 378, 385, and 394 of SEQ ID NO: 1. The mutations can be substitutions, deletions, or a combination thereof. The mutation at each position can independently be an alanine substitution, a glycine substitution, a serine substitution, a threonine substitution, or a deletion. In some embodiments, the substitution is an alanine. In some embodiments, the npr1 protein comprises the amino acid sequence of SEQ ID NO: 134.

In some embodiments, a npr1 protein comprises a NPR1 protein having mutations of one or more cysteines, wherein the cysteines are located in a region corresponding to residues 140-160 of SEQ ID NO: 1. In some embodiments, a npr1 protein comprises an A. thaliana NPR1 protein having mutations of one or more cysteines, wherein the cysteines are located in a region corresponding to residues 140-160 of SEQ ID NO: 1. In some embodiments, a npr1 protein comprises an ortholog of an A. thaliana NPR1 protein having mutations of one or more cysteines, wherein the cysteines are located in a region corresponding to residues 140-160 of SEQ ID NO: 1. The ortholog can be, but is not limited to, a Nicotiana benthamiana NPR1, a Solanum lycopersicon NPR1, a Vitis vinifera NPR1, a Hordeum vulgare NPR1, a Medicago truncatula NPR1, a Nicotiana tabacum NPR1, a Oryza sativa NPR1, a Glycine max NPR1, a Populus trichocarpa NPR1, a Cucumis melo NPR1, a Zea mays NPR1, a Raphanus sativus NPR1, a Brassica napus NPR1, a Brassica oleracea NPR1, a Brassica rapa NPR1, a Brassica juncea NPR1. The ortholog can be, but is not limited to, a protein comprising the amino acid sequence of SEQ ID NO: 2-12 and 30-34. The mutation can be a substitution, a deletion, or a combination thereof. The mutation at each position can independently be an alanine substitution, a glycine substitution, a serine substitution, a threonine substitution, or a deletion. In some embodiments, the substitution is an alanine.

In some embodiments, a npr1 protein comprises an A. thaliana NPR1 protein having a mutation of a cysteine at one or more of positions 150, 155, 156, and 160 of SEQ ID NO: 1. In some embodiments, a npr1 protein comprises an A. thaliana NPR1 protein having a mutation of a cysteine at position 150 of SEQ ID NO: 1; position 155 of SEQ ID NO: 1; position 156 of SEQ ID NO: 1; position 160 of SEQ ID NO: 1; positions 150 and 155 of SEQ ID NO: 1; positions 150 and 156 of SEQ ID NO: 1; positions 150 and 160 of SEQ ID NO: 1; positions 155 and 156 of SEQ ID NO: 1; positions 155 and 160 of SEQ ID NO: 1; positions 156 and 160 of SEQ ID NO: 1; positions 150, 155, and 156 of SEQ ID NO: 1; positions 150, 156, and 160 of SEQ ID NO: 1; positions 150, 155, and 156 of SEQ ID NO: 1; positions 155, 156, and 160 of SEQ ID NO: 1; or positions 150, 155, 156, and 160 of SEQ ID NO: 1. The mutation can be a substitution, a deletion, or a combination thereof. The mutation at each position can independently be an alanine substitution, a glycine substitution, a serine substitution, a threonine substitution, or a deletion. In some embodiments, the substitution is an alanine. In some embodiments, the npr1 protein comprises the amino acid sequence of SEQ ID NO: 134.

In some embodiments, a npr1 protein comprises a npr1 ACTD protein, a BTB domain npr1 protein, or a npr1 sim3 protein. A ACTD npr1 comprises a NPR1 protein having a deletion of amino acids corresponding to amino acids 1-364 of SEQ ID NO: 1 or an ortholog thereof. A BTB npr1 protein consists the BTB domain of NPR1 corresponding to amino acids 65-144 of SEQ ID NO: 1 or an ortholog thereof. A npr1sim3 protein comprises a SUMOylation-deficient mutant of NPR1.

In some embodiments, the npr1 protein has increased interaction with CUL3 compared to wild-type NPR1 in the absence of salicylic acid when measured under the same conditions. In some embodiments, the npr1 protein has increased interaction with CUL3 compared to wild-type NPR1 at lower concentrations of salicylic acid when measured under the same conditions Interaction may be determined using methods known in the art, including, but not limited to, co-immunoprecipitation, yeast two-hybrid assay, and BiFC assay.

### Nucleic Acids

Any of the described npr1 proteins that form salicylic acid-independent NPR1 condensates can be expressed in a plant or plant cell by introducing into the plant or plant cell or a progenitor plant or plant cell, a nucleic acid encoding the npr1 protein. Nucleic acids encoding the described npr1 proteins (npr1 genes) are readily made using methods known in the art. Nucleic acid sequences encoding A. thaliana NPR1 (SEQ ID NO: 1) and its orthologs, such as SEQ ID NOS: 2-12 and 30-34, are known in the art. Modification (mutation) of a nucleic acid sequence encoding a NPR1 gene to form a nucleic acid encoding a described npr1 protein can be done using methods known in the art for site directed mutagenesis of a nucleic acid.

A npr1 gene encoding a npr1 protein that forms salicylic acid-independent NPR1 condensates can be a nucleic acid encoding: (a) a protein comprising the amino acid sequence of any of SEQ ID NOS: 134-160 or an ortholog thereof; or (b) a protein having at least 70%, at least 75%, at least 80%, at least 85%, at least 90%, at least 95%, at least 96%, at least 97%, at least 98%, or at least 99% identity to a protein comprising the amino acid sequence of any of SEQ ID NOS: 134-160, wherein the protein retains salicylic acid-independent NPR1 condensate formation function.

A npr1 gene encoding any of the described npr1 proteins can be operably linked to one or more expression control elements (e.g., one or more heterologous expression control elements). The expression control elements can comprise a promoter (e.g., a heterologous promoter), one or more upstream open reading frames (uORFs) (e.g., one or more heterologous uORFs), or a promoter and one or more uORFs. In a specific example, a npr1 gene can be operably linked to a TBF1 promoter (e.g., Arabidopsis TBF1 promoter) and one or more TBF1 uORFs (e.g., Arabidopsis TBF1 uORF1 and/or uORF2).

The promoter can be, but is not limited to, a constitutive promoter, an inducible promoter, a temporally-regulated promoter, a developmentally regulated promoter, a chemically regulated promoter, a tissue-preferred promoter, a tissue-specific promoter, a TBF1 promoter, a 35S promoter, a ubiquitin promoter, a tCUP cryptic constitutive promoter, a Rsyn7 promoter, a pathogen-inducible promoter, a maize In2-2 promoter, a tobacco PR-1a promoter, a glucocorticoid-inducible promoter, an estrogen-inducible promoter, a tetracycline-inducible promoter, a tetracycline-repressible promoter, a T3 promoter, a T7 promoter, or a SP6 promoter. In a specific example, the promoter is a TBF1 promoter, such as an Arabidopsis TBF1 promoter. See, e.g., Pajerowska-Mukhtar et al. (2012) Curr. Biol. 22(2):103-112; Xu et al. (2017) Nature 545(7655):491-494; US 2018-0273965; U.S. Pat. No. 10,584,346; US 2015-0113685; U.S. Pat. No. 10,017,773; WO 2013/096567; US 2019-0352664; and WO 2018/144831, each of which is herein incorporated by reference in its entirety for all purposes. TBF1 is an important transcription factor for the growth-to-defense switch upon immune induction. In one example, the promoter can comprise the sequence set forth in SEQ ID NO: 167. In another example, the promoter can comprise the sequence set forth in SEQ ID NO: 168.

The upstream uORF can comprise one or more TBF1 gene uORFs. See, e.g., Pajerowska-Mukhtar et al. (2012) Curr. Biol. 22(2):103-112; Xu et al. (2017) Nature 545(7655):491-494; US 2018-0273965; U.S. Pat. No. 10,584,346; US 2015-0113685; U.S. Pat. No. 10,017,773; WO 2013/096567; US 2019-0352664; and WO 2018/144831, each of which is herein incorporated by reference in its entirety for all purposes. The TBF1 uORFs can comprise, for example, Arabidopsis TBF1 uORFs, such as uORF1 (SEQ ID NO: 162, or encoding SEQ ID NO: 164) and uORF2 (SEQ ID NO: 163, or encoding SEQ ID NO: 165). In one example, the uORFs can comprise uORF1 (SEQ ID NO: 162 or encoding SEQ ID NO: 164), uORF2 (SEQ ID NO: 163 or encoding SEQ ID NO: 165), or both uORF1 and uORF2. In a specific example, the npr1 gene can be operably linked to a regulatory sequence (e.g., 5′ regulatory sequence) comprising SEQ ID NO: 166, which includes both uORF1 and uORF2. In another specific example, the npr1 gene can be operably linked to a regulatory sequence (e.g., 5′ regulatory sequence) comprising SEQ ID NO: 168, which includes a TBF1 promoter, uORF1, and uORF2.

A nucleic acid encoding a npr1 protein may be introduced into a plant or plant cell using a number of methods known in the art, including but not limited to electroporation, DNA bombardment or biolistic approaches, lipofection, nucleofection, microinjection, via the use of various DNA-based vectors such as Agrobacterium tumefaciens and Agrobacterium rhizogenes vectors, and CRISPR or CRISPR/Cas9. Once a plant cell has been successfully transformed, it may be cultivated to regenerate a transgenic plant. Delivery can be to cells (e.g., in vitro or ex vivo administration) or target tissues (e.g., in vivo administration).

In some embodiments, Agrobacterium tumefaciens is used to generate a transgenic plant. There are numerous vectors designed for Agrobacterium transformation. For stable transformation, Agrobacterium systems can utilize “binary” vectors that permit plasmid manipulation in both E. coli and Agrobacterium, and typically contain one or more selectable markers to recover transformed plants. Binary vectors for use in Agrobacterium transformation systems typically comprise the borders of T-DNA, multiple cloning sites, replication functions for Escherichia coli and A. tumefaciens, and selectable marker and reporter genes. Agrobacterium-mediated transformation of a large number of plants are extensively described in the literature (see, for example, Agrobacterium Protocols, Wan, ed., Humana Press, 2^{nd} edition, 2006). Various methods for introducing DNA into Agrobacteria are known, including electroporation, freeze/thaw methods, and triparental mating.

Methods of producing a plant that expresses a npr1 protein as described herein or genetically modifying a plant to express a npr1 protein as described herein using a CRISPR/Cas system are described. Stress resistant plants created using a CRISPR/Cas system and nucleic acids for producing a stress resistant plant using a CRISPR/Cas system are also described.

A CRISPR system can comprise an RNA-guided DNA endonuclease enzyme and a guide RNA. The RNA-guided DNA endonuclease enzyme can be, but is not limited to, a Cas9 protein. A CRISPR system can comprise one or more nucleic acids encoding an RNA-guided DNA endonuclease enzyme (such as, but not limited to a Cas9 protein) and a guide RNA. A guide RNA can comprise a CRISPR RNA (crRNA) and a trans-activating CRISPR RNA (tracrRNA), either as separate molecules or a single chimeric guide RNA (sgRNA). The guide RNA contains a guide sequence having complementarity to a sequence in the target gene genomic region. The Cas protein can be introduced into the plant in the form of a protein or a nucleic acid (DNA or RNA) encoding the Cas protein (e.g., operably linked to a promoter expressible in the plant). The guide RNA can be introduced into the plant in the form of RNA or a DNA encoding the guide RNA (e.g., operably linked to a promoter expressible in the plant). In some embodiments, the CRISPR system can be delivered to a plant or plant cell via a bacterium. The bacterium can be, but is not limited to, Agrobacterium tumefaciens.

The CRISPR system is designed to target insertion of a nucleic acid encoding a npr1 protein into the plant genome. The CRIPSR system can be designed to target insertion of the nucleic acid encoding a npr1 protein into the NPR1 locus. The CRISPR/Cas system can be, but is not limited to, a CRISPR class 1 system, CRISPR class 2 system, CRISPR/Cas system, a CRISPR/Cas9 system, a CRISPR/zCas9 system or CRISPR/Cas3 system.

To transgenic plants may be used to generate subsequent generations (e.g., T₁, T₂, etc.) by selfing of primary or secondary transformants, or by sexual crossing of primary or secondary transformants with other plants (transformed or untransformed).

### Plant Cells and Plants

Plant cells including or expressing any of the npr1 proteins described herein are provided. The plants cells have increased stress tolerance, increased cell survival (decreased cell death) against biotic and/or abiotic stress, and/or increased cell survival against plant immune response, relative to a similar plant cell not expressing the nrp1 protein. The plant cell can be a monocot plant cell or a dicot plant cell. The plant cell can be, but is not limited to, a food crop plant cell, a biofuel plant cell, a corn plant cell, a legume plant cell, a bean plant cell, a rice plant cell, a soybean plant cell, a cotton plant cell, a sugarcane plant cell, a tobacco plant cell, a palm oil plant cell, a date palm cell, a wheat cell, a vegetable plant cell, a squash plant cell, a Solanaceae plant cell, a tomato cell, a banana plant cell, a potato plant cell, a pepper plant cell, a moss plant cell, a parsley plant cell, a sunflower plant cell, a mustard plant cell, a sorghum plant cell, a millet plant cell, a citrus plant cell, an apple plant cell, a strawberry plant cell, a rapeseed plant cell, a cabbage plant cell, a cassava plant cell, a coffee plant cell, a sweet potato plant cell, a jatropha plant cell, or a switchgrass plant cell. The npr1 gene can be integrated into the genome of the plant cell. The npr1 gene can be integrated into the genome of the plant cell at the NPR1 locus or a heterologous locus.

A plant cell can contain a npr1 gene encoding: (a) a protein comprising the amino acid sequence of any of SEQ ID NOS: 134-160 or an ortholog thereof; or (b) a protein having at least 70%, at least 75%, at least 80%, at least 85%, at least 90%, at least 95%, at least 96%, at least 97%, at least 98%, or at least 99% identity to a protein comprising the amino acid sequence of any of SEQ ID NOS: 134-160, wherein the protein retains salicylic acid-independent NPR1 condensate formation function.

In some embodiments, the plant cells expresses a wild-type NPR1 gene. In some embodiments, the plant cell does not express a wild-type NPR1 gene. For example, in some embodiments, the plant cell is npr1-null.

Plants including or expressing any of the npr1 proteins described herein are provided. The plant can be a monocot plant or a dicot plant. The plants have increased stress tolerance, increased cell survival (decreased cell death) against biotic and/or abiotic stress, and/or increased cell survival against plant immune response, relative to a similar plant not expressing the nrp1 protein. The plant can be, but is not limited to, a food crop plant, a biofuel plant, a corn plant, a legume plant, a bean plant, a rice plant, a soybean plant, a cotton plant, a sugarcane plant, a tobacco plant, a palm oil plant, a date palm, a wheat, a vegetable plant, a squash plant, a Solanaceae plant, a tomato, a banana plant, a potato plant, a pepper plant, a moss plant, a parsley plant, a sunflower plant, a mustard plant, a sorghum plant, a millet plant, a citrus plant, an apple plant, a strawberry plant, a rapeseed plant, a cabbage plant, a cassava plant, a coffee plant, a sweet potato plant, a jatropha plant, or a switchgrass plant. The npr1 gene can be integrated into the genome of the plant. The npr1 gene can be integrated into the genome of the plant at the NPR1 locus or a heterologous locus.

A plant can contain a npr1 gene encoding: (a) a protein comprising the amino acid sequence of any of SEQ ID NOS: 134-160 or an ortholog thereof; or (b) a protein having at least 70%, at least 75%, at least 80%, at least 85%, at least 90%, at least 95%, at least 96%, at least 97%, at least 98%, or at least 99% identity to a protein comprising the amino acid sequence of any of SEQ ID NOS: 134-160, wherein the protein retains salicylic acid-independent NPR1 condensate formation function.

In some embodiments, the plant expresses a wild-type NPR1 gene. In some embodiments, the plant does not express a wild-type NPR1 gene. For example, in some embodiments, the plant is npr1-null.

### Methods

Described are methods of increasing stress tolerance, increasing cell survival (decreasing cell death) against biotic and/or abiotic stress, and/or increasing cell survival against plant immune response in a plant or plant cell comprising expressing in the plant or plant cell a npr1 protein that forms salicylic acid-independent NPR1 condensates. The npr1 protein can be any of the npr1 proteins described herein. The methods comprise introducing into the plant, the plant cell, or a progenitor of the plant or plant cell, a nucleic acid encoding any of the described npr1 proteins such that the nucleic acid is expressed in the plant or plant cell. In some embodiments, the nucleic acid is operatively linked to one or more expression control elements that are functional in the plant or plant cell. In some embodiments, the nucleic acid is operatively linked to a promoter, or a promoter and one or more uORFs. In some embodiments, the nucleic acid comprises a npr1 gene encoding: (a) a protein comprising the amino acid sequence of any of SEQ ID NOS: 134-160 or an ortholog thereof; (b) a protein having at least 70%, at least 75%, at least 80%, at least 85%, at least 90%, at least 95%, at least 96%, at least 97%, at least 98%, or at least 99% identity to a protein comprising the amino acid sequence of any of SEQ ID NOS: 134-160, wherein the protein retains salicylic acid-independent NPR1 condensate formation function; (c) a npr1 ΔCTD protein; (d) a BTB domain npr1 protein, or (e) a npr1sim3 protein. Expression of the npr1 protein in the plant or plant cell results in the plant or plant cell having increased stress tolerance, increased cell survival (decreased cell death) against biotic and/or abiotic stress, and/or increased cell survival against plant immune response relative to a similar plant or plant cell that does not express the npr1 protein. A biotic stress can be, but is not limited to, a viral or bacterial infection. An abiotic stress can be, but is not limited to, high temperature (heat shock) stress, low temperature (cold shock) stress, oxidative stress, or DNA damage.

In some embodiments, increasing stress tolerance comprises one or more of: decreasing programmed cell death, decreasing effector-triggered immunity (ETI)-induced cell death, increasing formation of NPR1 condensates, and degrading EDS1 and specific WRKY transcription factors required for pathogen ETI.

The plant, plant cell, or progenitor of the plant or plant cell can be, but is not limited to, a monocot or a dicot. The plant, plant cell, or progenitor of the plant or plant cell can be, but is not limited to, a food crop plant, a biofuel plant, a corn plant, a legume plant, a bean plant, a rice plant, a soybean plant, a cotton plant, a sugarcane plant, a tobacco plant, a palm oil plant, a date palm, a wheat, a vegetable plant, a squash plant, a Solanaceae plant, a tomato, a banana plant, a potato plant, a pepper plant, a moss plant, a parsley plant, a sunflower plant, a mustard plant, a sorghum plant, a millet plant, a citrus plant, an apple plant, a strawberry plant, a rapeseed plant, a cabbage plant, a cassava plant, a coffee plant, a sweet potato plant, a jatropha plant, or a switchgrass plant, or a cell derived from any of these plants. The npr1 gene can be integrated into the genome of the plant or plant cell. The npr1 gene can be integrated into the genome of the plant or plant cell at the NPR1 locus or a heterologous locus.

The nucleic acid encoding the npr1 protein can be introduced into a plant, plant cell, or progenitor of the plant or plant cell that expresses a wild-type NPR1 gene. Alternatively, the nucleic acid encoding the npr1 protein can be introduced into a plant, plant cell, or progenitor of the plant or plant cell that does not express a wild-type NPR1 gene (e.g., a npr1-null plant, plant cell, or progenitor of the plant or plant cell). The nucleic acid encoding the npr1 protein can be introduced into a plant, or progenitor of the plant having one genotype and introgressed into a plant having a different genotype. “Introgression” of a gene or locus means introduction of the gene or locus from a donor plant comprising the gene or locus into a recipient plant by standard breeding techniques. Selection of can be done phenotypically or selection can be done with the use of genetic markers through marker-assisted breeding, or combinations of these. The process of introgressing is often referred to as “backcrossing” when the process is repeated two or more times. In introgressing or backcrossing, the “donor” parent refers to the parental plant with the desired gene or locus to be introgressed. The “recipient” parent refers to the parental plant into which the gene or locus is being introgressed. Selection is started in the F1 or any further generation from a cross between the recipient plant and the donor plant.

In some embodiments, producing a plant having increased stress tolerance, increased cell survival (decreased cell death) against biotic and/or abiotic stress, and/or increased cell survival against plant immune response comprises crossing a first plant expressing any of the described npr1 proteins with a second plant to produce at least a first progeny plant, and selecting one or more progeny plants that express the npr1 protein any or have increased stress tolerance, increased cell survival (decreased cell death) against biotic and/or abiotic stress, and/or increased cell survival against plant immune response compared to a control plant that doesn't express the npr1 protein.

Also described are methods of improving plant growth under conditions of stress, the methods comprising introducing into one or more plants a nucleic acid encoding any of the described npr1 proteins such that the npr1 protein is expressed in the plant, subjecting the one or more plants to stress; and selecting a plant having improved plant growth under the stress when compared to a plant that lacks the nucleic acid encoding the npr1 protein.

Another aspect of the present disclosure provides all that is described and illustrated herein.

The following Examples and attached Appendices are provided by way of illustration and not by way of limitation.

### Brief Description of the Sequences

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 2
Description of Sequences.

| SEQ | | |
|---|---|---|
| ID | | |
| NO | Type | Description |
| 1 | Protein | Arabidopsis thaliana npr1 protein |
| | | MDTTIDGFADSYEISSTSFVATDNTDSSIVYLAAEQVLTGPDVSALQLLSNSFESVFDSP |
| | | DDFYSDAKLVLSDGREVSFHRCVLSARSSFFKSALAAAKKEKDSNNTAAVKLELKEIAKD |
| | | YEVGFDSVVTVLAYVYSSRVRPPPKGVSECADENCCHVACRPAVDFMLEVLYLAFIFKIP |
| | | ELITLYQRHLLDVVDKVVIEDTLVILKLANICGKACMKLLDRCKEIIVKSNVDMVSLEKS |
| | | LPEELVKEIIDRRKELGLEVPKVKKHVSNVHKALDSDDIELVKLLLKEDHTNLDDACALH |
| | | FAVAYCNVKTATDLLKLDLADVNHRNPRGYTVLHVAAMRKEPQLILSLLEKGASASEATL |
| | | EGRTALMIAKQATMAVECNNIPEQCKHSLKGRLCVEILEQEDKREQIPRDVPPSFAVAAD |
| | | ELKMTLLDLENRVALAQRLFPTEAQAAMEIAEMKGTCEFIVTSLEPDRLTGTKRTSPGVK |
| | | IAPFRILEEHQSRLKALSKTVELGKRFFPRCSAVLDQIMNCEDLTQLACGEDDTAEKRLQ |
| | | KKQRYMEIQETLKKAFSEDNLELGNSSLTDSTSSTSKSTGGKRSNRKLSHRRR |
| 2 | Protein | Nicotiana benthamiana npr1 protein |
| | | MDNSGTAFSDSNDISGSSSICCIGGGMTESFSPETSPAEITSLKRLSETLESIFDAASPE |
| | | FDYFADAKLVIPGAGKEIPVHRCILSARSPFFKNLFCGKKEKNSNKVELKEIMKEYEVSY |
| | | DGVVSVLAYLYSGKIRPSPKDVCVCVDNDCSHVACRPAVAFLAEVLYTSFTFQISELVDK |
| | | FQRHLLDILDKIAADDVMVVLSVANICGKACERLLSSCIEIIVKSNVDIITLDKALPHDI |
| | | VKQITDSRAELGLQGPESNGFPDKHVKRIHRALDSDDVELLQMLLREGHTTLDDAFALHY |
| | | AVAYCDAKTTAELLDLALAEINHQNSRGYTVLHVAAMRKEPKIIVSLLTKGARPSDLTSD |
| | | GRKALQIAKRLTRLVDFSKSPEEGKSASKDRLCIEILEQAERRDPLLGEASVSLAMAGDD |
| | | LRMKLLYLENRVGLAKLLFPMEAKVAMDIAQVDGTSEFPLASINKKMVNAQRTTVDLNEV |
| | | PFKIKEEHLNRLRALSRTVELGKRFFPRCSEVLNKIMDADDLSEIAYMGNDTAEERQLKK |
| | | QRYMELQEILSKAFTEDKEEFDMTNNISSSCSSTSKGVDKPNKLPFRK |
| 3 | Protein | Solanum lycopersicon npr1 protein |
| | | MDSRTAFSDSNDISGSSSICCMNESETSLADVNSLKRLSETLESIFDASAPDFDFFADAK |
| | | LLAPGGKEIPVHRCILSARSPFFKNVFCGKDSNTKLELKELMKEYEVSFDAVVSVLAYLY |
| | | SGKVRPASKDVCVCVDNECLHVACRPAVAFMVQVLYASFTFQISQLVDKFQRHLLDILDK |
| | | AVADDVMMVLSVANICGKACERLLSRCIDIIVKSNVDIITLDKSLPHDIVKQITDSRAEL |
| | | GLQGPESNGFPDKHVKRIHRALDSDDVELLRMLLKEGHTTLDDAYALHYAVAYCDAKTTA |
| | | ELLDLSLADVNHQNPRGHTVLHVAAMRKEPKIIVSLLTKGARPSDLTSDGKKALQIAKRL |
| | | TRLVDFTKSTEEGKSAPKDRLCIEILEQAERRDPLLGEASLSLAMAGDDLRMKLLYLENR |
| | | VGLAKLLFPMEAKVAMDIAQVDGTSELPLASMRKKIADAQRTTVDLNEAPFKMKEEHLNR |
| | | LRALSRTVELGKRFFPRCSEVLNKIMDADDLSEIAYMGNDTVEERQLKKQRYMELQEILS |
| | | KAFTEDKEEFAKTNMSSSCSSTSKGVDKPNNLPFRK |
| 4 | Protein | Vitis vinifera npr1 protein |
| | | MDYRAALSDSNDFSGSSSICCIAATTESLSSEVSPPDISALRRLSENLESVFESPEFDFF |
| | | TDARIVVAGGREVPVHRCILAARSVFFKAVLAGARKEKEAKFELKDLAKEFDVGYDSLVA |
| | | VLGYLYSGRVGALPKGVCACVDDDCPHSACRPAVDFMVEVLYASFAFQISELVGLYQRRL |
| | | MDILDKVASDDILVILSVANLCGKACDRLLARCIDIIIKSDVDVVTLERALPQEMVKQIV |
| | | DSRLELGFEEPESTNFPDKHVKRIHRALDSDDVELVRMLLKEGHTTLDDAYALHYAVAFG |
| | | DAKTTTELLDLGLADVNHKNHRGYTVLHIAAMRKEPKIIVSLLTKGARPTDITPDGRNAL |
| | | QIAKRLTRAVDYHKSTEEGKPSPKDQLCVEVLEQAERRDPLIGEASFSLAIAGDDLRMKL |
| | | LYLENRVGLAKLLFPMEAKVAMDIAQVDGTSEFTLTAIRPRNLADAQRTTVDLNEAPFRI |
| | | KEEHLNRLRALSKTVDLGKRFFPRCSEVLNKIMDADDLSDLAYLGNGTTEERLLKKRRYK |
| | | ELQDQLCKAFNEDKEENDKSRISSSSSSTSLGFGRNNSRLSCKK |
| 5 | Protein | Hordeum vulgare npr1 protein |
| | | MEAPSSHVTTSFSDCDSVSMEDAAPDADVEALRRLSDNLAAAFRSPDDFAFLADARFAVP |
| | | GAPDLCVHRCVLSARSPFLRALFKRRAAAAGSAGGAEGDRVELRELLGGEVEVGYEALRL |
| | | VLDYLYSGRVCDLPKTACACVDEGGCAHVGCHPAVSFMAQVLFAASTFQVGELASLFQRH |
| | | LLDLLDKVEADNLPLVLSVANLCNKSCVKLFERCLERVVRSDLDMITLDKALPLDVIKQI |
| | | IDSRITLGLASPEDNGFPNKHVRRILSALDSDDVELVRLLLKEGQTNLDDAFALHYAVEH |
| | | CDSKITTELLDIALADVNLRNPRGYTVLHIAARRRDPKIVVSLLTKGARPSDFTFDGRKA |
| | | VQIAKRLTKHGDYFGNTEEGKPSPNDKLCIEILEEAERRDPQLGEASVSLALAGDCLRGK |
| | | LLYLENRVALARIMFPIEARVAMDIAQVDGTLEFTLGSCTNPPPEITTVDLNDTPFKMKD |
| | | EHLARMRALSKTVELGKRFFPRCSNVLDKIMDDEPELASLGRDASSERKRRFHDLHDTLL |
| | | KAFSEDKEEFARSATLSASSSSTPTVARNLTGRPRR |
| 6 | Protein | Medicago truncatula npr1 protein |
| | | MMYLRSGFSEYSNEISNNTSSELCCTTAPNSTITASQDVITFTQPLNRLSDNLASILDDT |
| | | GFDFFSDAKIIAKDGREVSVHRCILSARSSFFKDVFKGKKETTLQLKEVAKDYDVGFDAL |
| | | NVVLRYLYSERVEDHHLSAKDVCVCVDDDCLHFGCWPVVDFMLQLLYASFTFQISELLAL |
| | | YQDHLLDILDKMAIDDMLVVLSIANICGKTCDKLLKRCTDIIVESNVDITTLEKSLPQSI |
| | | VKLVTYKRKQLGLDMYETVNLLDKHVTRIHRALDSDDVELVRLLLKEGHTTLDEAHALHY |
| | | AVAYCDVKTTTELLDLGLADVNHKNLRGYSVLHVAAKRKEPKIIVSLLTKGAQPSELTMD |
| | | GRKALQISKRCTKAVDYYKSTEEGKVSSNDRLCIEILEQAERREPLHGEASLSLAKAGDD |
| | | LRMKLLYLENRVGLAKLLFPMEAKVVMDITPIDGTSEFTPNLGGYQRTTMDLNEAPFKIK |
| | | EEHLIRMKALSRAVELGKRFFPRCSEVLNKIMDADDLSQLACMGHDSPEDRQVKRRRYAE |
| | | LQEVLNKVFHEDKEEFDKSGMSSSSSSTSIGMPRANNSMIAMNH |
| 7 | Protein | Nicotiana tabacum npr1 protein |
| | | MDNSRTAFSDSNDISGSSSICCIGGGMTEFFSPETSPAEITSLKRLSETLESIFDASLPE |
| | | FDYFADAKLVVSGPCKEIPVHRCILSARSPFFKNLFCGKKEKNSSKVELKEVMKEHEVSY |
| | | DAVMSVLAYLYSGKVRPSPKDVCVCVDNDCSHVACRPAVAFLVEVLYTSFTFQISELVDK |
| | | FQRHLLDILDKTAADDVMMVLSVANICGKACERLLSSCIEIIVKSNVDIITLDKALPHDI |
| | | VKQITDSRAELGLQGPESNGFPDKHVKRIHRALDSDDVELLQMLLREGHTTLDDAYALHY |
| | | AVAYCDAKTTAELLDLALADINHQNSRGYTVLHVAAMRKEPKIVVSLLTKGARPSDLTSD |
| | | GRKALQIAKRLTRLVDFSKSPEEGKSASNDRLCIEILEQAERRDPLLGEASVSLAMAGDD |
| | | LRMKLLYLENRVGLAKLLFPMEAKVAMDIAQVDGTSEFPLASIGKKMANAQRTTVDLNEA |
| | | PFKIKEEHLNRLRALSRTVELGKRFFPRCSEVLNKIMDADDLSEIAYMGNDTAEERQLKK |
| | | QRYMELQEILTKAFTEDKEEYDKTNNISSSCSSTSKGVDKPNKLPFRK |
| 8 | Protein | Oryza sativa npr1 protein |
| | | MEPPTSHVTNAFSDSDSASVEEGGADADADVEALRRLSDNLAAAFRSPEDFAFLADARIA |
| | | VPGGGGGGGDLLVHRCVLSARSPFLRGVFARRAAAAAGGGGEDGGERLELRELLGGGGEE |
| | | VEVGYEALRLVLDYLYSGRVGDLPKAACLCVDEDCAHVGCHPAVAFMAQVLFAASTFQVA |
| | | ELTNLFQRRLLDVLDKVEVDNLLLILSVANLCNKSCMKLLERCLDMVVRSNLDMITLEKS |
| | | LPPDVIKQIIDARLSLGLISPENKGFPNKHVRRIHRALDSDDVELVRMLLTEGQTNLDDA |
| | | FALHYAVEHCDSKITTELLDLALADVNHRNPRGYTVLHIAARRREPKIIVSLLTKGARPA |
| | | DVTFDGRKAVQISKRLTKQGDYFGVTEEGKPSPKDRLCIEILEQAERRDPQLGEASVSLA |
| | | MAGESLRGRLLYLENRVALARIMFPMEARVAMDIAQVDGTLEFNLGSGANPPPERQRTTV |
| | | DLNESPFIMKEEHLARMTALSKTVELGKRFFPRCSNVLDKIMDDETDPVSLGRDTSAEKR |
| | | KRFHDLQDVLQKAFHEDKEENDRSGLSSSSSSTSIGAIRPRR |
| 9 | Protein | Glycine max npr1 protein |
| | | MNFRSGSSDSKDASNSSTGEAYLSGVSDVITPLRRLSEQLGSILDGGGVDFFSDAKIVAG |
| | | DGREVAVNRCILAARSGFFKHVFAGGGGCVLRLKEVAKDYNVGLEALGIVLAYLYSGRVK |
| | | PLPQGGVCVCVDDVCSHFGCRPAIDFLLQLLYASSTFQLNELIALXQGHLLDILEKVAID |
| | | DILVVLSVANICGIVCERLLARCTEMILKSDADITTLEKALPQHLVKQITDKRIELDLYM |
| | | PENFNFPDKHVNRIHRALDSDDVELVRLLLKEGHTTLDDAYALHYAVAYCDVKTTTELLD |
| | | LGLADVNHKNYRGYSVLHVAAMRKEPKIIVSLLTKGAQPSDLTLDGRKALQISKRLTKAV |
| | | DYYKSTEEGKVSCSDRLCIEILEQAERREPLLGEASLSLAMAGDDLRMKLLYLENRVGLA |
| | | KVLFPMEAKVIMDISQIDGTSEFPSTDMYCPNISDHQRTTVDLNDAPFRMKEEHLVRLRA |
| | | LSRTVELGKRFFPRCSEVLNKIMDADDLTQLTCMGDDSPEDRLRKRRRYVELQEVLNKVF |
| | | NEDKEEFDRSAMSSSSSSTSIGVVRPNANLAMKN |
| 10 | Protein | Populus trichocarpa npr1 protein |
| | | MDNRIGFSDSNEISNGSSTCCIETPSTSKPFTNPEIVALQQLSGNLEAIFDSQDFDYFAD |
| | | AKITSSNYNREVPVHRCILSARSPFFKSVFSSPVAKDRSGVAKFELKELAKDYDVGFDSL |
| | | MTVLGYLYCGKVRPWPKDVCACVDDDCSHIACRPAVDLLTEVLYASFTFQVNELVALYQR |
| | | HLLDILDKVSTDDILVILAVANICGEACERLLTRCVEIIVKSNVDIVTLDKALPQYIVKK |
| | | IMDSRLELGLNVPENSNLLDKHVKRIHRALDSDDVELVRMLLKEAHTNLDDAHALHYAVS |
| | | YCDAKTTTEILDLGLADVNCRNSRGYTVLHVAAMRKDPKIIVSLLTKGARLSDLTLDGRK |
| | | ALQISKRLTRAMDYHKSTEEGKASPKERLCIEILEQAERRDPLLGEASLSLAMAGDDLRM |
| | | KLLYLENRVGLAKLLFPMEAKVAMDIAQVDGTSEFPLAGIRPSILSGAQRGAVDLNEAPF |
| | | RMHEEHLNRMRALSRTVELGKRFFPRCSDVLNKIMDADDLSQIAYLGNETSEERLVKRQR |
| | | HLELQDALSKAFNEDKQEFDRSVISSSSSTKSIGTARSNGKLIDMGGGH |
| 11 | Protein | Cucumis melo npr1 protein |
| | | MADSHEPSSSLSFTSSSYTNGSQSCNMSPSSISDPMPSLEVISLNKLSSNLAQLLIHDGC |
| | | DYTDADIVVEGVPVGIHRCILAVRSRFFHYLFQKDEKPAMKDGKPQYHMNELLPYGKVGH |
| | | EAFLILLSYLYSGKLNSSPANVSTCVDNSCAHDACGPAIDFAVQLTYASSIFQIPELVSL |
| | | FQRHLLNYVVKALVENVIQILVVAFHCQLSPLVTQCIDRIARSDLDCASLEKGLPYEVAE |
| | | RIKLVRLKSRGGDEQNLVADSPRDKKIKKICLALDSDDVELMKLLLSESDVTLDEANALH |
| | | YAAAYCDPKSLTEVLNLNIADVNLRNSRGYTVLHVAAMRKDPSVIMSLLNKGAWAFDLTP |
| | | DGRTAANICQRLTRPKDYHAKTEKGQETNKDRLCIDILEREMWRNPTSDSSILSLAMADD |
| | | VHMKLIYLENRVAFARLLFPSEARLAMDIANADTTSEFVGLSMPKNSNKNLREVNLNETP |
| | | SVQNKRFLSRMQALLKTVEMGRRFFPNCSEALDKFVADDLPDLFYLEKGTVEEQRIKRKR |
| | | FKELKNDVQKAFDKDKAAKLNQSGLSPSSSSTSLKHGTNHRNVRRQ |
| 12 | Protein | Zea mays npr1 protein |
| | | MEPMDSQLTALALSDSDSASVEGAAADAADADLQALRRLSDNLAAAFRSPDDFAFLADAR |
| | | IVVPGAPDLRVHRCVLCARSPFLRDAFARRAASAGEEEKDKDSYMCKVELRDLLGDEVEV |
| | | GYDALRLVLDYLYSGRVAALPKAACLCVDEDACAHVGCRPAVAFMAQVLFAASTFDVAEL |
| | | TNLFQRRLLDVLDKVEVDNLPLVLSVANLCSKSCVKLLERCLDVVVRSNLDMIALEKKLP |
| | | PDVVKEIVDARVSLGLVSPEDKGFPNIHVRRIHRALDSDDVELVRMLLKEGKTNLDDAYA |
| | | LHYAVEHCDSKITTELLDLALADVNHRNPRGYTVLHIAAMRREPKIIVSLLTKGARPSDL |
| | | TFDDRKAVQISKRLTKHGDYFGPTEDGKPSPKDRLCIEVLEQAERRDPQLGEASVSLAIE |
| | | GDSARGRLLYLENRVALARILFPMEARVAMDIAQVDGTLEFTLVSSVNLPAEIQRTVDLN |
| | | DTPFTMKEEHLARMRALSKTVEVGKRFFPRCSKVLDTIMDDEAEMASLGRDTSAEKKRRF |
| | | HDLQDLVQKAFSEDKEENDRSAARSPSSSSTTTTSIGAVRPRR |
| 13 | Protein | Arabidopsis thaliana RDR2 sequence alignment |
| 14 | Protein | Nicotiana benthamiana RDR2 sequence alignment |
| 15 | Protein | Solanum lycopersicon RDR2 sequence alignment |
| 16 | Protein | Vitis vinifera RDR2 sequence alignment |
| 17 | Protein | Hordeum vulgare RDR2 sequence alignment |
| 18 | Protein | Medicago truncatula RDR2 sequence alignment |
| 19 | Protein | Nicotiana tabacum RDR2 sequence alignment |
| 20 | Protein | Oryza sativa RDR2 sequence alignment |
| 21 | Protein | Glycine max RDR2 sequence alignment |
| 22 | Protein | Populus trichocarpa RDR2 sequence alignment |
| 23 | Protein | Cucumis melo RDR2 sequence alignment |
| 24 | Protein | Zea mays RDR2 sequence alignment |
| 25 | Protein | Raphanus sativus RDR2 sequence alignment |
| 26 | Protein | Brassica napus RDR2 sequence alignment |
| 27 | Protein | Brassica oleracea RDR2 sequence alignment |
| 28 | Protein | Brassica rapa RDR2 sequence alignment |
| 29 | Protein | Brassica juncea RDR2 sequence alignment |
| 30 | Protein | Brassica napus npr1 protein |
| | | METIARFDDFYEISSTSFPAAPAPTNNSGSSTVFPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADESCCHVACRPAVDFMVEVLYLAFVFQIQEL |
| | | VTMYQRHLLDVVDKVMIEDTLVVLKLANICGKACKKLFDKCREIIVKSNVDVVTLKKSLP |
| | | ENIAKQVIDIRKELGLEVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAECCILEKGKLAAKGGVCVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEFGKRFFPRCSKVLDDIVDSEDLTILALVEEDTPEQRQQKRQRFMEIQEIVQM |
| | | AFSKDKEDLGKSSLSASSSSTSKLTGTDSRGFFGIIFYMLQPSVHHLKKVSRVPPRTTTS |
| | | VPSNPEGL |
| 31 | Protein | Brassica rapa npr1 protein |
| | | METIAGFDDFYEISSTSFLAAPAPTDNSGSSTVYPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADDSCCHVACRPAVDFMVEVLYLAFVFQIQEL |
| | | VTMYQRHLLDVVDKVNIEDTLVVLKLANICGKACKKLFDKCREIIVKSNVDVVTLKKSLP |
| | | ENIAKQVIDIRKELGLEVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAECCILEKGKLAAKGGVCVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEFGKRFFPRCSKVLDDIVDSEDLTILALVEEDTPEQRQQKRQRFMEIQEIVQM |
| | | AFSKDKEDLGKSSLSASSSSTSKLTGKKRSIAKPSHRRR |
| 32 | Protein | Brassica oleracea npr1 protein |
| | | METIAGFDDFYEISSTSFLAAPAPTDNSGSSTVYPTELFTRPEVSAFQLLSNSLESVFDS |
| | | PEAFYSDAKLVLSDDKEVSFHRCILSARSLFFKAALTAAEKVQKSTPVKLELKTLAAEYD |
| | | VGFDSVVAVLAYVYSGRVRPPPKGVSECADESCCHVACRPAVDFMVEVLYLAFVFQIQEL |
| | | VTMYQRHLLDVVDKVMIEDTLVVLKLANICGKACKKLFDKCREIIVKSNVDVVTLKKSLP |
| | | EDIAKQVIDIRKELGLEVAEPEKHVSNIHKALESDDLDLVVMLLKEGHTNLDEAYALHFA |
| | | VAYCDEKTARNLLELGFADVNRRNPRGYTVIHVAAMRKEPTLIALLLTKGANALEMSLDG |
| | | RTALLIAKQVTKAAECCILEKGKLAAKGGVCVEILKQPDNTREPFPEDVSPSLAVAADQF |
| | | KIRLIDLENRVQMARCLYPMEAQVAMDFARMKGTREFVVTTATDLHMEPFKFVEMHQSRL |
| | | TALSKTGIIKSFVTKITETNLCLTDLVFLLFLPVEFGKRFFPRCSKVLDDIVDSEDLTIL |
| | | ALVEEDTPEQRQQKRQRFMEIQEIVQMAFSKDKEDLGKSSLSASSSSTSKLTGKKRSIAK |
| | | PSHRRR |
| 33 | Protein | Raphanus sativus npr1 protein |
| | | METIAGFDDFYEISSTSFLAAPAPTDNSGSSTVYPTELLTTRPEVSASQLLSNSLESVFD |
| | | SPEEFYSDAKLVLSDDREVSFHRCILSARSPFFKAELAAAEKVQKSTPVKLELKKLAAEY |
| | | DVGFDSVVAVLAYVYCGRVRPPPKGVSECADESCCHVACRPAVDFMVEVLYLAFVEQIPE |
| | | LVTMYQRHLLDVIEKVIIEDTLVVLKLANICGKACKKLFDKCKEIIVMSDVDVVTLKKSL |
| | | PEDVAKQVIDIRKELGLEVAEPEKHVSNIHKALESDDLALVDMLLNEGHTNLDDAYALHF |
| | | AVAYCDVQTAKDLLELELADVNRRNPRGYTVLHVAAMRKEPTLIALLLTKGANASETSLD |
| | | GRTALVIAKQVTKATECCILERGKLSAKGGVCVEILKEPDNKREPFPEDVSPSLAVAADE |
| | | LKIRLIDLENRVQMARCLYPMEAQVAMDFARMKGTHEFVVTTTTDLNMEPFKFVEMHRSR |
| | | LTALSKTVEFGKRFFPRCSKVLDDILNFEDLTILALVEEETPEQRQQKRQRFMEIQEIVR |
| | | MAFSKDKEDLGKSSLSPSSSSTSKLNGKKRSIAKLSHRRRR |
| 34 | Protein | Brassica juncea npr1 protein |
| | | METIARFDDFYEISSTSFPAAPAPTDNSGSSTVYPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADDSCCHVACRPAVDFMVEVLYLAFVFQIQEL |
| | | VTMYQRHLLDVVDKVNIEDTLVVLKLANICGKACKKLFDKCREIIVKSNVDVVTLKKSLP |
| | | ENIAKQVIDIRKELGLDVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAECCILEKGKLAAKGGVCVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEFGKRFFPRCSKVLDDIVDSEDLTILALVEEDTPEQRQQKRQRFMEIQEIVQM |
| | | AFSKDKEDLGKSSLSASSSSTSKLTGKKRSIAKPSHRRR |
| 35- | DNA | Primers |
| 133 | | |
| 134 | Protein | A. thaliana npr1 rdr2 protein |
| | | MDTTIDGFADSYEISSTSFVATDNTDSSIVYLAAEQVLTGPDVSALQLLSNSFESVFDSP |
| | | DDFYSDAKLVLSDGREVSFHRCVLSARSSFFKSALAAAKKEKDSNNTAAVKLELKEIAKD |
| | | YEVGFDSVVTVLAYVYSSRVRPPPKGVSECADENCCHVACRPAVDFMLEVLYLAFIFKIP |
| | | ELITLYQRHLLDVVDKVVIEDTLVILKLANICGKACMKLLDRCKEIIVKSNVDMVSLEKS |
| | | LPEELVKEIIDRRKELGLEVPKVKKHVSNVHKALDSDDIELVKLLLKEDHTNLDDACALH |
| | | FAVAYCNVKTATDLLKLDLADVNHRNPRGYTVLHVAAMRKEPQLILSLLEKGASASEATL |
| | | EGRTALMIAKQATMAVEANNIPEQAKHSLKGRLAVEILEQEDKREQIPRDVPPSFAVAAD |
| | | ELKMTLLDLENRVALAQRLFPTEAQAAMEIAEMKGTCEFIVTSLEPDRLTGTKRTSPGVK |
| | | IAPFRILEEHQSRLKALSKTVELGKRFFPRCSAVLDQIMNCEDLTQLACGEDDTAEKRLQ |
| | | KKQRYMEIQETLKKAFSEDNLELGNSSLTDSTSSTSKSTGGKRSNRKLSHRRR |
| 135 | Protein | A. thaliana npr1 rdrl protein |
| | | MDTTIDGFADSYEISSTSFVATDNTDSSIVYLAAEQVLTGPDVSALQLLSNSFESVFDSP |
| | | DDFYSDAKLVLSDGREVSFHRCVLSARSSFFKSALAAAKKEKDSNNTAAVKLELKEIAKD |
| | | YEVGFDSVVTVLAYVYSSRVRPPPKGVSEAADENAAHVAARPAVDFMLEVLYLAFIFKIP |
| | | ELITLYQRHLLDVVDKVVIEDTLVILKLANICGKACMKLLDRCKEIIVKSNVDMVSLEKS |
| | | LPEELVKEIIDRRKELGLEVPKVKKHVSNVHKALDSDDIELVKLLLKEDHTNLDDACALH |
| | | FAVAYCNVKTATDLLKLDLADVNHRNPRGYTVLHVAAMRKEPQLILSLLEKGASASEATL |
| | | EGRTALMIAKQATMAVECNNIPEQCKHSLKGRLCVEILEQEDKREQIPRDVPPSFAVAAD |
| | | ELKMTLLDLENRVALAQRLFPTEAQAAMEIAEMKGTCEFIVTSLEPDRLTGTKRTSPGVK |
| | | IAPFRILEEHQSRLKALSKTVELGKRFFPRCSAVLDQIMNCEDLTQLACGEDDTAEKRLQ |
| | | KKQRYMEIQETLKKAFSEDNLELGNSSLTDSTSSTSKSTGGKRSNRKLSHRRR |
| 136 | Protein | A. thaliana npr1 rdr3 protein |
| | | MDTTIDGFADSYEISSTSFVATDNTDSSIVYLAAEQVLTGPDVSALQLLSNSFESVFDSP |
| | | DDFYSDAKLVLSDGREVSFHRCVLSARSSFFKSALAAAKKEKDSNNTAAVKLELKEIAKD |
| | | YEVGFDSVVTVLAYVYSSRVRPPPKGVSECADENCCHVACRPAVDFMLEVLYLAFIFKIP |
| | | ELITLYQRHLLDVVDKVVIEDTLVILKLANICGKACMKLLDRCKEIIVKSNVDMVSLEKS |
| | | LPEELVKEIIDRRKELGLEVPKVKKHVSNVHKALDSDDIELVKLLLKEDHTNLDDACALH |
| | | FAVAYCNVKTATDLLKLDLADVNHRNPRGYTVLHVAAMRKEPQLILSLLEKGASASEATL |
| | | EGRTALMIAKQATMAVECNNIPEQCKHSLKGRLCVEILEQEDKREQIPRDVPPSFAVAAD |
| | | ELKMTLLDLENRVALAQRLFPTEAQAAMEIAEMKGTCEFIVTSLEPDRLTGTKRTSPGVK |
| | | IAPFRILEEHQSRLKALSKTVELGKRFFPRASAVLDQIMNAEDLTQLAAGEDDTAEKRLQ |
| | | KKQRYMEIQETLKKAFSEDNLELGNSSLTDSTSSTSKSTGGKRSNRKLSHRRR |
| 137 | Protein | Nicotiana benthamiana npr1 rdr2 protein |
| | | MDNSGTAFSDSNDISGSSSICCIGGGMTESFSPETSPAEITSLKRLSETLESIF |
| | | DAASPEFDYFADAKLVIPGAGKEIPVHRCILSARSPFFKNLFCGKKEKNSNKVELKEIMK |
| | | EYEVSYDGVVSVLAYLYSGKIRPSPKDVCVCVDNDCSHVACRPAVAFLAEVLYTSFTFQI |
| | | SELVDKFQRHLLDILDKIAADDVMVVLSVANICGKACERLLSSCIEIIVKSNVDIITLDK |
| | | ALPHDIVKQITDSRAELGLQGPESNGFPDKHVKRIHRALDSDDVELLQMLLREGHTTLDD |
| | | AFALHYAVAYCDAKTTAELLDLALAEINHQNSRGYTVLHVAAMRKEPKIIVSLLTKGARP |
| | | SDLTSDGRKALQIAKRLTRLVDFSKSPEEGKSASKDRLAIEILEQAERRDPLLGEASVSL |
| | | AMAGDDLRMKLLYLENRVGLAKLLFPMEAKVAMDIAQVDGTSEFPLASINKKMVNAQRTT |
| | | VDLNEVPFKIKEEHLNRLRALSRTVELGKRFFPRCSEVLNKIMDADDLSEIAYMGNDTAE |
| | | ERQLKKQRYMELQEILSKAFTEDKEEFDMTNNISSSCSSTSKGVDKPNKLPFRK |
| 138 | Protein | Solanum lycopersicon npr1 rdr2 protein |
| | | MDSRTAFSDSNDISGSSSICCMNESETSLADVNSLKRLSETLESIFDASAPDFDFFADAK |
| | | LLAPGGKEIPVHRCILSARSPFFKNVFCGKDSNTKLELKELMKEYEVSFDAVVSVLAYLY |
| | | SGKVRPASKDVCVCVDNECLHVACRPAVAFMVQVLYASFTFQISQLVDKFQRHLLDILDK |
| | | AVADDVMMVLSVANICGKACERLLSRCIDIIVKSNVDIITLDKSLPHDIVKQITDSRAEL |
| | | GLQGPESNGFPDKHVKRIHRALDSDDVELLRMLLKEGHTTLDDAYALHYAVAYCDAKTTA |
| | | ELLDLSLADVNHQNPRGHTVLHVAAMRKEPKIIVSLLTKGARPSDLTSDGKKALQIAKRL |
| | | TRLVDFTKSTEEGKSAPKDRLAIEILEQAERRDPLLGEASLSLAMAGDDLRMKLLYLENR |
| | | VGLAKLLFPMEAKVAMDIAQVDGTSELPLASMRKKIADAQRTTVDLNEAPFKMKEEHLNR |
| | | LRALSRTVELGKRFFPRCSEVLNKIMDADDLSEIAYMGNDTVEERQLKKQRYMELQEILS |
| | | KAFTEDKEEFAKTNMSSSCSSTSKGVDKPNNLPFRK |
| 139 | Protein | Vitis vinifera npr1 rdr2 protein |
| | | MDYRAALSDSNDFSGSSSICCIAATTESLSSEVSPPDISALRRLSENLESVFESPEFDFF |
| | | TDARIVVAGGREVPVHRCILAARSVFFKAVLAGARKEKEAKFELKDLAKEFDVGYDSLVA |
| | | VLGYLYSGRVGALPKGVCACVDDDCPHSACRPAVDFMVEVLYASFAFQISELVGLYQRRL |
| | | MDILDKVASDDILVILSVANLCGKACDRLLARCIDIIIKSDVDVVTLERALPQEMVKQIV |
| | | DSRLELGFEEPESTNFPDKHVKRIHRALDSDDVELVRMLLKEGHTTLDDAYALHYAVAFG |
| | | DAKTTTELLDLGLADVNHKNHRGYTVLHIAAMRKEPKIIVSLLTKGARPTDITPDGRNAL |
| | | QIAKRLTRAVDYHKSTEEGKPSPKDQLAVEVLEQAERRDPLIGEASFSLAIAGDDLRMKL |
| | | LYLENRVGLAKLLFPMEAKVAMDIAQVDGTSEFTLTAIRPRNLADAQRTTVDLNEAPFRI |
| | | KEEHLNRLRALSKTVDLGKRFFPRCSEVLNKIMDADDLSDLAYLGNGTTEERLLKKRRYK |
| | | ELQDQLCKAFNEDKEENDKSRISSSSSSTSLGFGRNNSRLSCKK |
| 140 | Protein | Hordeum vulgare npr1 rdr2 protein |
| | | MEAPSSHVTTSFSDCDSVSMEDAAPDADVEALRRLSDNLAAAFRSPDDFAFLADARFAVP |
| | | GAPDLCVHRCVLSARSPFLRALFKRRAAAAGSAGGAEGDRVELRELLGGEVEVGYEALRL |
| | | VLDYLYSGRVCDLPKTACACVDEGGCAHVGCHPAVSFMAQVLFAASTFQVGELASLFQRH |
| | | LLDLLDKVEADNLPLVLSVANLCNKSCVKLFERCLERVVRSDLDMITLDKALPLDVIKQI |
| | | IDSRITLGLASPEDNGFPNKHVRRILSALDSDDVELVRLLLKEGQTNLDDAFALHYAVEH |
| | | CDSKITTELLDIALADVNLRNPRGYTVLHIAARRRDPKIVVSLLTKGARPSDFTFDGRKA |
| | | VQIAKRLTKHGDYFGNTEEGKPSPNDKLAIEILEEAERRDPQLGEASVSLALAGDCLRGK |
| | | LLYLENRVALARIMFPIEARVAMDIAQVDGTLEFTLGSCTNPPPEITTVDLNDTPFKMKD |
| | | EHLARMRALSKTVELGKRFFPRCSNVLDKIMDDEPELASLGRDASSERKRRFHDLHDTLL |
| | | KAFSEDKEEFARSATLSASSSSTPTVARNLTGRPRR |
| 141 | Protein | Medicago truncatula npr1 rdr2 protein |
| | | MMYLRSGFSEYSNEISNNTSSELCCTTAPNSTITASQDVITFTQPLNRLSDNLASILDDT |
| | | GFDFFSDAKIIAKDGREVSVHRCILSARSSFFKDVFKGKKETTLQLKEVAKDYDVGFDAL |
| | | NWLRYLYSERVEDHHLSAKDVCVCVDDDCLHFGCWPVVDFMLQLLYASFTFQISELLAL |
| | | YQDHLLDILDKMAIDDMLVVLSIANICGKTCDKLLKRCTDIIVESNVDITTLEKSLPQSI |
| | | VKLVTYKRKQLGLDMYETVNLLDKHVTRIHRALDSDDVELVRLLLKEGHTTLDEAHALHY |
| | | AVAYCDVKTTTELLDLGLADVNHKNLRGYSVLHVAAKRKEPKIIVSLLTKGAQPSELTMD |
| | | GRKALQISKRATKAVDYYKSTEEGKVSSNDRLAIEILEQAERREPLHGEASLSLAKAGDD |
| | | LRMKLLYLENRVGLAKLLFPMEAKVVMDITPIDGTSEFTPNLGGYQRTTMDLNEAPFKIK |
| | | EEHLIRMKALSRAVELGKRFFPRCSEVLNKIMDADDLSQLACMGHDSPEDRQVKRRRYAE |
| | | LQEVLNKVFHEDKEEFDKSGMSSSSSSTSIGMPRANNSMIAMNH |
| 142 | Protein | Nicotiana tabacum npr1 rdr2 protein |
| | | MDNSRTAFSDSNDISGSSSICCIGGGMTEFFSPETSPAEITSLKRLSETLESIFDASLPE |
| | | FDYFADAKLVVSGPCKEIPVHRCILSARSPFFKNLFCGKKEKNSSKVELKEVMKEHEVSY |
| | | DAVMSVLAYLYSGKVRPSPKDVCVCVDNDCSHVACRPAVAFLVEVLYTSFTFQISELVDK |
| | | FQRHLLDILDKTAADDVMMVLSVANICGKACERLLSSCIEIIVKSNVDIITLDKALPHDI |
| | | VKQITDSRAELGLQGPESNGFPDKHVKRIHRALDSDDVELLQMLLREGHTTLDDAYALHY |
| | | AVAYCDAKTTAELLDLALADINHQNSRGYTVLHVAAMRKEPKIVVSLLTKGARPSDLTSD |
| | | GRKALQIAKRLTRLVDFSKSPEEGKSASNDRLAIEILEQAERRDPLLGEASVSLAMAGDD |
| | | LRMKLLYLENRVGLAKLLFPMEAKVAMDIAQVDGTSEFPLASIGKKMANAQRTTVDLNEA |
| | | PFKIKEEHLNRLRALSRTVELGKRFFPRCSEVLNKIMDADDLSEIAYMGNDTAEERQLKK |
| | | QRYMELQEILTKAFTEDKEEYDKTNNISSSCSSTSKGVDKPNKLPFRK |
| 143 | Protein | Oryza sativa npr1 rdr2 protein |
| | | MEPPTSHVTNAFSDSDSASVEEGGADADADVEALRRLSDNLAAAFRSPEDFAFLADARIA |
| | | VPGGGGGGGDLLVHRCVLSARSPFLRGVFARRAAAAAGGGGEDGGERLELRELLGGGGEE |
| | | VEVGYEALRLVLDYLYSGRVGDLPKAACLCVDEDCAHVGCHPAVAFMAQVLFAASTFQVA |
| | | ELTNLFQRRLLDVLDKVEVDNLLLILSVANLCNKSCMKLLERCLDMVVRSNLDMITLEKS |
| | | LPPDVIKQIIDARLSLGLISPENKGFPNKHVRRIHRALDSDDVELVRMLLTEGQTNLDDA |
| | | FALHYAVEHCDSKITTELLDLALADVNHRNPRGYTVLHIAARRREPKIIVSLLTKGARPA |
| | | DVTFDGRKAVQISKRLTKQGDYFGVTEEGKPSPKDRLAIEILEQAERRDPQLGEASVSLA |
| | | MAGESLRGRLLYLENRVALARIMFPMEARVAMDIAQVDGTLEFNLGSGANPPPERQRTTV |
| | | DLNESPFIMKEEHLARMTALSKTVELGKRFFPRCSNVLDKIMDDETDPVSLGRDTSAEKR |
| | | KRFHDLQDVLQKAFHEDKEENDRSGLSSSSSSTSIGAIRPRR |
| 144 | Protein | Glycine max npr1 rdr2 protein |
| | | MNFRSGSSDSKDASNSSTGEAYLSGVSDVITPLRRLSEQLGSILDGGGVDFFSDAKIVAG |
| | | DGREVAVNRCILAARSGFFKHVFAGGGGCVLRLKEVAKDYNVGLEALGIVLAYLYSGRVK |
| | | PLPQGGVCVCVDDVCSHFGCRPAIDFLLQLLYASSTFQLNELIALXQGHLLDILEKVAID |
| | | DILVVLSVANICGIVCERLLARCTEMILKSDADITTLEKALPQHLVKQITDKRIELDLYM |
| | | PENFNFPDKHVNRIHRALDSDDVELVRLLLKEGHTTLDDAYALHYAVAYCDVKTTTELLD |
| | | LGLADVNHKNYRGYSVLHVAAMRKEPKIIVSLLTKGAQPSDLTLDGRKALQISKRLTKAV |
| | | DYYKSTEEGKVSCSDRLAIEILEQAERREPLLGEASLSLAMAGDDLRMKLLYLENRVGLA |
| | | KVLFPMEAKVIMDISQIDGTSEFPSTDMYCPNISDHQRTTVDLNDAPFRMKEEHLVRLRA |
| | | LSRTVELGKRFFPRCSEVLNKIMDADDLTQLTCMGDDSPEDRLRKRRRYVELQEVLNKVF |
| | | NEDKEEFDRSAMSSSSSSTSIGVVRPNANLAMKN |
| 145 | Protein | Populus trichocarpa npr1 rdr2 protein |
| | | MDNRIGFSDSNEISNGSSTCCIETPSTSKPFTNPEIVALQQLSGNLEAIFDSQDFDYFAD |
| | | AKITSSNYNREVPVHRCILSARSPFFKSVFSSPVAKDRSGVAKFELKELAKDYDVGFDSL |
| | | MTVLGYLYCGKVRPWPKDVCACVDDDCSHIACRPAVDLLTEVLYASFTFQVNELVALYQR |
| | | HLLDILDKVSTDDILVILAVANICGEACERLLTRCVEIIVKSNVDIVTLDKALPQYIVKK |
| | | IMDSRLELGLNVPENSNLLDKHVKRIHRALDSDDVELVRMLLKEAHTNLDDAHALHYAVS |
| | | YCDAKTTTEILDLGLADVNCRNSRGYTVLHVAAMRKDPKIIVSLLTKGARLSDLTLDGRK |
| | | ALQISKRLTRAMDYHKSTEEGKASPKERLAIEILEQAERRDPLLGEASLSLAMAGDDLRM |
| | | KLLYLENRVGLAKLLFPMEAKVAMDIAQVDGTSEFPLAGIRPSILSGAQRGAVDLNEAPF |
| | | RMHEEHLNRMRALSRTVELGKRFFPRCSDVLNKIMDADDLSQIAYLGNETSEERLVKRQR |
| | | HLELQDALSKAFNEDKQEFDRSVISSSSSTKSIGTARSNGKLIDMGGGH |
| 146 | Protein | Cucumis melo npr1 rdr2 protein |
| | | MADSHEPSSSLSFTSSSYTNGSQSCNMSPSSISDPMPSLEVISLNKLSSNLAQLLIHDGC |
| | | DYTDADIVVEGVPVGIHRCILAVRSRFFHYLFQKDEKPAMKDGKPQYHMNELLPYGKVGH |
| | | EAFLILLSYLYSGKLNSSPANVSTCVDNSCAHDACGPAIDFAVQLTYASSIFQIPELVSL |
| | | FQRHLLNYVVKALVENVIQILVVAFHCQLSPLVTQCIDRIARSDLDCASLEKGLPYEVAE |
| | | RIKLVRLKSRGGDEQNLVADSPRDKKIKKICLALDSDDVELMKLLLSESDVTLDEANALH |
| | | YAAAYCDPKSLTEVLNLNIADVNLRNSRGYTVLHVAAMRKDPSVIMSLLNKGAWAFDLTP |
| | | DGRTAANICQRLTRPKDYHAKTEKGQETNKDRLAIDILEREMWRNPTSDSSILSLAMADD |
| | | VHMKLIYLENRVAFARLLFPSEARLAMDIANADTTSEFVGLSMPKNSNKNLREVNLNETP |
| | | SVQNKRFLSRMQALLKTVEMGRRFFPNCSEALDKFVADDLPDLFYLEKGTVEEQRIKRKR |
| | | FKELKNDVQKAFDKDKAAKLNQSGLSPSSSSTSLKHGTNHRNVRRQ |
| 147 | Protein | Zea mays npr1 rdr2 protein |
| | | MEPMDSQLTALALSDSDSASVEGAAADAADADLQALRRLSDNLAAAFRSPDDFAFLADAR |
| | | IVVPGAPDLRVHRCVLCARSPFLRDAFARRAASAGEEEKDKDSYMCKVELRDLLGDEVEV |
| | | GYDALRLVLDYLYSGRVAALPKAACLCVDEDACAHVGCRPAVAFMAQVLFAASTFDVAEL |
| | | TNLFQRRLLDVLDKVEVDNLPLVLSVANLCSKSCVKLLERCLDVVVRSNLDMIALEKKLP |
| | | PDVVKEIVDARVSLGLVSPEDKGFPNIHVRRIHRALDSDDVELVRMLLKEGKTNLDDAYA |
| | | LHYAVEHCDSKITTELLDLALADVNHRNPRGYTVLHIAAMRREPKIIVSLLTKGARPSDL |
| | | TFDDRKAVQISKRLTKHGDYFGPTEDGKPSPKDRLAIEVLEQAERRDPQLGEASVSLAIE |
| | | GDSARGRLLYLENRVALARILFPMEARVAMDIAQVDGTLEFTLVSSVNLPAEIQRTVDLN |
| | | DTPFTMKEEHLARMRALSKTVEVGKRFFPRCSKVLDTIMDDEAEMASLGRDTSAEKKRRF |
| | | HDLQDLVQKAFSEDKEENDRSAARSPSSSSTTTTSIGAVRPRR |
| 148 | Protein | Brassica napus npr1 rdr2 protein |
| | | METIARFDDFYEISSTSFPAAPAPTNNSGSSTVFPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADESCCHVACRPAVDFMVEVLYLAFVFQIQEL |
| | | VTMYQRHLLDVVDKVMIEDTLVVLKLANICGKACKKLFDKCREIIVKSNVDVVTLKKSLP |
| | | ENIAKQVIDIRKELGLEVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAECCILEKGKLAAKGGVAVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEFGKRFFPRCSKVLDDIVDSEDLTILALVEEDTPEQRQQKRQRFMEIQEIVQM |
| | | AFSKDKEDLGKSSLSASSSSTSKLTGTDSRGFFGIIFYMLQPSVHHLKKVSRVPPRTTTS |
| | | VPSNPEGL |
| 149 | Protein | Brassica rapa npr1 rdr2 protein |
| | | METIAGFDDFYEISSTSFLAAPAPTDNSGSSTVYPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADDSCCHVACRPAVDFMVEVLYLAFVFQIQEL |
| | | VTMYQRHLLDVVDKVNIEDTLVVLKLANICGKACKKLFDKCREIIVKSNVDVVTLKKSLP |
| | | ENIAKQVIDIRKELGLEVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAECCILEKGKLAAKGGVAVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEFGKRFFPRCSKVLDDIVDSEDLTILALVEEDTPEQRQQKRQRFMEIQEIVQM |
| | | AFSKDKEDLGKSSLSASSSSTSKLTGKKRSIAKPSHRRR |
| 150 | Protein | Brassica oleracea npr1 rdr2 protein |
| | | METIAGFDDFYEISSTSFLAAPAPTDNSGSSTVYPTELFTRPEVSAFQLLSNSLESVFDS |
| | | PEAFYSDAKLVLSDDKEVSFHRCILSARSLFFKAALTAAEKVQKSTPVKLELKTLAAEYD |
| | | VGFDSVVAVLAYVYSGRVRPPPKGVSECADESCCHVACRPAVDFMVEVLYLAFVFQIQEL |
| | | VTMYQRHLLDVVDKVMIEDTLVVLKLANICGKACKKLFDKCREIIVKSNVDVVTLKKSLP |
| | | EDIAKQVIDIRKELGLEVAEPEKHVSNIHKALESDDLDLVVMLLKEGHTNLDEAYALHFA |
| | | VAYCDEKTARNLLELGFADVNRRNPRGYTVIHVAAMRKEPTLIALLLTKGANALEMSLDG |
| | | RTALLIAKQVTKAAECCILEKGKLAAKGGVAVEILKQPDNTREPFPEDVSPSLAVAADQF |
| | | KIRLIDLENRVQMARCLYPMEAQVAMDFARMKGTREFVVTTATDLHMEPFKFVEMHQSRL |
| | | TALSKTGIIKSFVTKITETNLCLTDLVFLLFLPVEFGKRFFPRCSKVLDDIVDSEDLTIL |
| | | ALVEEDTPEQRQQKRQRFMEIQEIVQMAFSKDKEDLGKSSLSASSSSTSKLTGKKRSIAK |
| | | PSHRRR |
| 151 | Protein | Raphanus sativus npr1 rdr2 protein |
| | | METIAGFDDFYEISSTSFLAAPAPTDNSGSSTVYPTELLTTRPEVSASQLLSNSLESVFD |
| | | SPEEFYSDAKLVLSDDREVSFHRCILSARSPFFKAELAAAEKVQKSTPVKLELKKLAAEY |
| | | DVGFDSVVAVLAYVYCGRVRPPPKGVSECADESCCHVACRPAVDFMVEVLYLAFVEQIPE |
| | | LVTMYQRHLLDVIEKVIIEDTLVVLKLANICGKACKKLFDKCKEIIVMSDVDVVTLKKSL |
| | | PEDVAKQVIDIRKELGLEVAEPEKHVSNIHKALESDDLALVDMLLNEGHTNLDDAYALHF |
| | | AVAYCDVQTAKDLLELELADVNRRNPRGYTVLHVAAMRKEPTLIALLLTKGANASETSLD |
| | | GRTALVIAKQVTKATECCILERGKLSAKGGVAVEILKEPDNKREPFPEDVSPSLAVAADE |
| | | LKIRLIDLENRVQMARCLYPMEAQVAMDFARMKGTHEFVVTTTTDLNMEPFKFVEMHRSR |
| | | LTALSKTVEFGKRFFPRCSKVLDDILNFEDLTILALVEEETPEQRQQKRQRFMEIQEIVR |
| | | MAFSKDKEDLGKSSLSPSSSSTSKLNGKKRSIAKLSHRRRR |
| 152 | Protein | Brassica juncea npr1 rdr2 protein |
| | | METIARFDDFYEISSTSFPAAPAPTDNSGSSTVYPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADDSCCHVACRPAVDFMVEVLYLAFVFQIQEL |
| | | VTMYQRHLLDVVDKVNIEDTLVVLKLANICGKACKKLFDKCREIIVKSNVDVVTLKKSLP |
| | | ENIAKQVIDIRKELGLDVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAECCILEKGKLAAKGGVAVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEFGKRFFPRCSKVLDDIVDSEDLTILALVEEDTPEQRQQKRQRFMEIQEIVQM |
| | | AFSKDKEDLGKSSLSASSSSTSKLTGKKRSIAKPSHRRR |
| 153 | Protein | A. thaliana npr1 rdr2 protein v2 |
| | | MDTTIDGFADSYEISSTSFVATDNTDSSIVYLAAEQVLTGPDVSALQLLSNSFESVFDSP |
| | | DDFYSDAKLVLSDGREVSFHRCVLSARSSFFKSALAAAKKEKDSNNTAAVKLELKEIAKD |
| | | YEVGFDSVVTVLAYVYSSRVRPPPKGVSECADENCCHVACRPAVDFMLEVLYLAFIFKIP |
| | | ELITLYQRHLLDVVDKVVIEDTLVILKLANICGKACMKLLDRCKEIIVKSNVDMVSLEKS |
| | | LPEELVKEIIDRRKELGLEVPKVKKHVSNVHKALDSDDIELVKLLLKEDHTNLDDACALH |
| | | FAVAYCNVKTATDLLKLDLADVNHRNPRGYTVLHVAAMRKEPQLILSLLEKGASASEATL |
| | | EGRTALMIAKQATMAVECNNIPEQCKHSLKGRLAVEILEQEDKREQIPRDVPPSFAVAAD |
| | | ELKMTLLDLENRVALAQRLFPTEAQAAMEIAEMKGTCEFIVTSLEPDRLTGTKRTSPGVK |
| | | IAPFRILEEHQSRLKALSKTVELGKRFFPRCSAVLDQIMNCEDLTQLACGEDDTAEKRLQ |
| | | KKQRYMEIQETLKKAFSEDNLELGNSSLTDSTSSTSKSTGGKRSNRKLSHRRR |
| 154 | Protein | Glycine max npr1 rdr2 protein v2 |
| | | MNFRSGSSDSKDASNSSTGEAYLSGVSDVITPLRRLSEQLGSILDGGGVDFFSDAKIVAG |
| | | DGREVAVNRCILAARSGFFKHVFAGGGGCVLRLKEVAKDYNVGLEALGIVLAYLYSGRVK |
| | | PLPQGGVCVCVDDVCSHFGCRPAIDFLLQLLYASSTFQLNELIALXQGHLLDILEKVAID |
| | | DILVVLSVANICGIVCERLLARCTEMILKSDADITTLEKALPQHLVKQITDKRIELDLYM |
| | | PENFNFPDKHVNRIHRALDSDDVELVRLLLKEGHTTLDDAYALHYAVAYCDVKTTTELLD |
| | | LGLADVNHKNYRGYSVLHVAAMRKEPKIIVSLLTKGAQPSDLTLDGRKALQISKRLTKAV |
| | | DYYKSTEEGKVSASDRLAIEILEQAERREPLLGEASLSLAMAGDDLRMKLLYLENRVGLA |
| | | KVLFPMEAKVIMDISQIDGTSEFPSTDMYCPNISDHQRTTVDLNDAPFRMKEEHLVRLRA |
| | | LSRTVELGKRFFPRCSEVLNKIMDADDLTQLTCMGDDSPEDRLRKRRRYVELQEVLNKVF |
| | | NEDKEEFDRSAMSSSSSSTSIGVVRPNANLAMKN |
| 155 | Protein | Cucumis melo npr1 rdr2 protein v2 |
| | | MADSHEPSSSLSFTSSSYTNGSQSCNMSPSSISDPMPSLEVISLNKLSSNLAQLLIHDGC |
| | | DYTDADIVVEGVPVGIHRCILAVRSRFFHYLFQKDEKPAMKDGKPQYHMNELLPYGKVGH |
| | | EAFLILLSYLYSGKLNSSPANVSTCVDNSCAHDACGPAIDFAVQLTYASSIFQIPELVSL |
| | | FQRHLLNYVVKALVENVIQILVVAFHCQLSPLVTQCIDRIARSDLDCASLEKGLPYEVAE |
| | | RIKLVRLKSRGGDEQNLVADSPRDKKIKKICLALDSDDVELMKLLLSESDVTLDEANALH |
| | | YAAAYCDPKSLTEVLNLNIADVNLRNSRGYTVLHVAAMRKDPSVIMSLLNKGAWAFDLTP |
| | | DGRTAANIAQRLTRPKDYHAKTEKGQETNKDRLAIDILEREMWRNPTSDSSILSLAMADD |
| | | VHMKLIYLENRVAFARLLFPSEARLAMDIANADTTSEFVGLSMPKNSNKNLREVNLNETP |
| | | SVQNKRFLSRMQALLKTVEMGRRFFPNCSEALDKFVADDLPDLFYLEKGTVEEQRIKRKR |
| | | FKELKNDVQKAFDKDKAAKLNQSGLSPSSSSTSLKHGTNHRNVRRQ |
| 156 | Protein | Brassica napus npr1 rdr2 protein v2 |
| | | METIARFDDFYEISSTSFPAAPAPTNNSGSSTVFPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADESCCHVACRPAVDFMVEVLYLAFVFQIQEL |
| | | VTMYQRHLLDVVDKVMIEDTLVVLKLANICGKACKKLFDKCREIIVKSNVDVVTLKKSLP |
| | | ENIAKQVIDIRKELGLEVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAEAAILEKGKLAAKGGVAVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEEGKREEPRCSKVLDDIVDSEDLTILALVEEDTPEQRQQKRQREMEIQEIVQM |
| | | AFSKDKEDLGKSSLSASSSSTSKLTGTDSRGFFGIIFYMLQPSVHHLKKVSRVPPRTTTS |
| | | VPSNPEGL |
| 157 | Protein | Brassica rapa npr1 rdr2 protein v2 |
| | | METIAGFDDFYEISSTSFLAAPAPTDNSGSSTVYPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADDSCCHVACRPAVDFMVEVLYLAFVFQIQEL |
| | | VTMYQRHLLDVVDKVNIEDTLVVLKLANICGKACKKLFDKCREIIVKSNVDVVTLKKSLP |
| | | ENIAKQVIDIRKELGLEVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAEAAILEKGKLAAKGGVAVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEFGKRFFPRCSKVLDDIVDSEDLTILALVEEDTPEQRQQKRQRFMEIQEIVQM |
| | | AFSKDKEDLGKSSLSASSSSTSKLTGKKRSIAKPSHRRR |
| 158 | Protein | Brassica oleracea npr1 rdr2 protein v2 |
| | | METIAGFDDFYEISSTSFLAAPAPTDNSGSSTVYPTELFTRPEVSAFQLLSNSLESVFDS |
| | | PEAFYSDAKLVLSDDKEVSFHRCILSARSLFFKAALTAAEKVQKSTPVKLELKTLAAEYD |
| | | VGFDSVVAVLAYVYSGRVRPPPKGVSECADESCCHVACRPAVDFMVEVLYLAFVFQIQEL |
| | | VTMYQRHLLDVVDKVMIEDTLVVLKLANICGKACKKLFDKCREIIVKSNVDVVTLKKSLP |
| | | EDIAKQVIDIRKELGLEVAEPEKHVSNIHKALESDDLDLVVMLLKEGHTNLDEAYALHFA |
| | | VAYCDEKTARNLLELGFADVNRRNPRGYTVIHVAAMRKEPTLIALLLTKGANALEMSLDG |
| | | RTALLIAKQVTKAAEAAILEKGKLAAKGGVAVEILKQPDNTREPFPEDVSPSLAVAADQF |
| | | KIRLIDLENRVQMARCLYPMEAQVAMDFARMKGTREFVVTTATDLHMEPFKFVEMHQSRL |
| | | TALSKTGIIKSFVTKITETNLCLTDLVFLLFLPVEFGKRFFPRCSKVLDDIVDSEDLTIL |
| | | ALVEEDTPEQRQQKRQRFMEIQEIVQMAFSKDKEDLGKSSLSASSSSTSKLTGKKRSIAK |
| | | PSHRRR |
| 159 | Protein | Raphanus sativus npr1 rdr2 protein v2 |
| | | METIAGFDDFYEISSTSFLAAPAPTDNSGSSTVYPTELLTTRPEVSASQLLSNSLESVFD |
| | | SPEEFYSDAKLVLSDDREVSFHRCILSARSPFFKAELAAAEKVQKSTPVKLELKKLAAEY |
| | | DVGFDSVVAVLAYVYCGRVRPPPKGVSECADESCCHVACRPAVDFMVEVLYLAFVEQIPE |
| | | LVTMYQRHLLDVIEKVIIEDTLVVLKLANICGKACKKLFDKCKEIIVMSDVDVVTLKKSL |
| | | PEDVAKQVIDIRKELGLEVAEPEKHVSNIHKALESDDLALVDMLLNEGHTNLDDAYALHF |
| | | AVAYCDVQTAKDLLELELADVNRRNPRGYTVLHVAAMRKEPTLIALLLTKGANASETSLD |
| | | GRTALVIAKQVTKATEAAILERGKLSAKGGVAVEILKEPDNKREPFPEDVSPSLAVAADE |
| | | LKIRLIDLENRVQMARCLYPMEAQVAMDFARMKGTHEFVVTTTTDLNMEPFKFVEMHRSR |
| | | LTALSKTVEFGKRFFPRCSKVLDDILNFEDLTILALVEEETPEQRQQKRQRFMEIQEIVR |
| | | MAFSKDKEDLGKSSLSPSSSSTSKLNGKKRSIAKLSHRRRR |
| 160 | Protein | Brassica juncea npr1 rdr2 protein v2 |
| | | METIARFDDFYEISSTSFPAAPAPTDNSGSSTVYPTELLTRPEVSAFQLLSNSLESVFDS |
| | | PEAFYSDAKLVLSDDKEVSFHRCILSARSLFFKAALAAAEKVQKSTPVKLELKTLAAEYD |
| | | VGFDSVVAVLAYVYSGRVRPPPKGVSECADDSCCHVACRPAVDFMVEVLYLAFVFQIQEL |
| | | VTMYQRHLLDVVDKVNIEDTLVVLKLANICGKACKKLFDKCREIIVKSNVDVVTLKKSLP |
| | | ENIAKQVIDIRKELGLDVAEPEKHVSNIHKALESDDLDLVVMLLKEGHTNLDEAYALHFA |
| | | VAYCDEKTARNLLELGLADVNRRNPRGYTVLHVAAMRKEPTLIALLLTKGANALETSLDG |
| | | RTALLIAKQVTKAAEAAILEKGKLAAKGGVAVEILKQPDNKREPFPEDVFPSLAVAADEF |
| | | KIRLIDLENRVQMARCLYPMEAQVAMDFARMKGTREFVVTTATDLHMEPFKFVEMHQSRL |
| | | TALSKTVEFGKRFFPRCSKVLDDIVDSEDLTILALVEEDTPEQRQQKRQRFMEIQEIVQM |
| | | AFSKDKEDLGKSSLSASSSSTSKLTGKKRSIAKPSHRRR |
| 161 | Protein | SV40 nuclear localization signal |
| | | CGGGPKKKRKVED |
| 162 | DNA | Arabidopsis thaliana TBF1 uORF1 |
| | | ATGGTCGTCGTCTTCATCTTCTTCCTCCATCATCAGATTTTTCCTTAA |
| 163 | DNA | Arabidopsis thaliana TBF1 uORF2 |
| | | ATGGAAGAAACCAAACGAAACTCCGATCTTCTCCGTTCTCGTGTTTTCCTCTCTGGCTTT |
| | | TATTGCTGGGATTGGGAATTTCTCACCGCTCTCTTGCTTTTTAGTTGCTGA |
| 164 | Protein | Protein encoded by Arabidopsis thaliana TBF1 uORF1 |
| | | MVVVFIFFLHHQIFP |
| 165 | Protein | Protein encoded by Arabidopsis thaliana TBF1 uORF2 |
| | | MEETKRNSDLLRSRVFLSGFYCWDWEFLTALLLFSC |
| 166 | DNA | Arabidopsis thaliana TBF1 5′ regulatory sequence with uORF1 and uORF2 |
| | | TCTAGAAACAGCATCCGTTTTTATAATTTAATTTTCTTACAAAGGTAGGACCAACATTTG |
| | | TGATCTATAAATCTTCCTACTACGTTATATAGAGACCCTTCGACATAACACTTAACTCGT |
| | | TTATATATTTGTTTTACTTGTTTTGCACATACACACAAAAATAAAAAAGACTTTATATTT |
| | | ATTTACTTTTTAATCACACGGATTAGCTCCGGCGAAGTATGGTCGTCGTCTTCATCTTCT |
| | | TCCTCCATCATCAGATTTTTCCTTAAATGGAAGAAACCAAACGAAACTCCGATCTTCTCC |
| | | GTTCTCGTGTTTTCCTCTCTGGCTTTTATTGCTGGGATTGGGAATTTCTCACCGCTCTCT |
| | | TGCTTTTTAGTTGCTGATTCTTTTTCCTTCGACTTTCTATTTCCAATCTTTCTTCTTCTC |
| | | TTTGTGTATTAGATTATTTTTAGTTTTATTTTTCTGTGGTAAAATAAAAAAAGTTCGCCG |
| | | GAG |
| 167 | DNA | Arabidopsis thaliana TBF1 promoter |
| | | CGACGACTAGTTTACAGAGAATTTGGACCGTCCGATGTAAAGCGAAAATAGATCTAGGTT |
| | | TTCCACGTGTCCCCTATTTTAATGAAACCTTCTGATTCATGTAGAAGTTTTACTCAATTT |
| | | AATATTTTTTAGTATGTAGTTTTGTGTGTGTGTGTGTGTGTGTTTTTATGGCTCCACACC |
| | | AACTTTTAAAATGGTAGAAGCATGTTGCATGTGATCGAGTAAAAAGCCAATAATGAGATT |
| | | CAGAAAAATAAAAATTACTTATATAGTTTTTTAGAGAAAAAATTGTATTTTGTTTAAAGC |
| | | CTTAATCCGGTTGTTGAAAGAGCTGTGTCACGAGTTAAAAATATTTTCTTTTCATTTTTT |
| | | AAGTAATTAGTTTATAATGCAAAAATGGTTTTTATTTATTTGTCTTCGCTTATAGAACTG |
| | | CAAATTGAGAGAGAAAAAAATGAATTAGTGGTGGTGACCAAACATTCAGGAAGCTGTGAT |
| | | TGATCATTTGTTTTTGAGGTGAGTGTAGTGGCAACGTATGACGTTAACATATGGCGTACA |
| | | TAATAATTAGATGAACTTAATCATAATAATCATATTGCATTTAATTCATATATCATATCC |
| | | CATTAGTTGGACCACTTGATTTGAGGTCATGAGAAGAACATTTATGTTTTTTTTAGTTTG |
| | | AATCGGAGTGATCACTAAAAACTAGATACTGAAAATTTTCAAACTAAAATCATATTAATC |
| | | TTCAAAAAATGTGAAATCTAAAAAAAAAAAAAATTTTAACGCGTTCATTGTAGCCAAGTA |
| | | GCCAAGTATTGTTAAAGTAGTAGTAAAAGAAGTTTAGCTTTAAGTGATATAATTTGACAC |
| | | AAATCCTACTTAGATATGGATAATAGGATATAGCTTCATGTATATTTTTATCGTTGCTTC |
| | | TGTAACCCCAAAATGTGTTGATATAAGCATTTGAATATTCGTATGTATAATGTTTTCTTT |
| | | TCACCGTAAAACATATTACAATGTTAGTTTATATTGGATTTTGAATGTGTTTATGAACAG |
| | | TTTTTGTCGACTCAAAAGTTAAGATGAGAATATGGAAGAAAGTAAAGTTTAAAAGTCATG |
| | | ATGGGAACAAGGAATGGAACTCAAACATTCTAATACTCAACAAACGCAATTATATTATTA |
| | | CCATGACTCATCTTTCAAGTTCCATCAAAAAGATTCGTGGAAAATAATAGACTTACGTTT |
| | | CAAATCCATGTTTCTTTCTTTATAACAAAAAAAATGGATGTTTCTTGACGCGTGTCGAGA |
| | | GTACTCACCATTACTCTGACTTCAGTGAGTTTGGTCAAGTGGTCTTTTTTTTTCTCATGT |
| | | CACCAAAGGTCCAAACCCTAGAAATTAGTTCGAACTTTCCATAGAAGAACTGAATAAATG |
| | | GTCCAAAATTGTTTTAAAAAGGACCTAAGCCATTAGTTCATTGAATTCGAGTTAATGGGT |
| | | GAAGATTTTTATGATAACGAAAGTCGGAGTAATTATGCTTTTGGTCCGATAGTTTTCTAA |
| | | TTTGTTTTCTTTCCATTTTTTTTTTTTCAAATACTACATACTATATAAGATAGTGGTTTG |
| | | TGTTAATGTCATCGATGTGTTACCATCCGCATTATATTAATTATTTATCCCAACATAAAG |
| | | TCAGAATCTGTAATTTCTTTGTTATAAAATACAGTAAATGGTTCCGTTTAAGCTGTTAGA |
| | | TGATTTTTGAGTAAAAACTAATGTAAAAAAAACAAAAAAAAAACAATGTAGTTCATAATA |
| | | CATGCATGTTTTAAAGAAGTTTCTTGTTTACTATCAACTTGAATAGTATTTCACGAAGTC |
| | | AAAATTGTTCATTCCGACTTTTCTATGTGGAGAAAAAAAATTCTATCATTGTGCACAATT |
| | | TAACAGAATGTAATTTCTTGTAAAAGAAGAGGAAACAATTCGCTGTTAGTAAATGTGAAG |
| | | TATAGAAGTCTAAAATGAGATACCTCAACTAGCTTGAATTAAGAAAAAAAACAAAAACTC |
| | | TATCGACATGAAAAAGGTCGCAAATATTTATCATTTATCAATGCCAAAGGAGTATTTGGT |
| | | TCACAAAATACTGAATCATTTATATAGATATATAATTAGCTCTAAATTCTACTATAACTT |
| | | GCAAAATAAGTATACTGACTCAATTATATAGCGTTTAAAAATAGACGATTTGTATGATGA |
| | | GGTCCATATATATGGAGATGTGCATGCAACTATCGACATTTTCACACGTTGATATCGTCT |
| | | TTCTCCAATGGAGACTTGAATTTGTGTAAACTATGAATACTCGTCTCTCTAAGACCTTTT |
| | | TTCTTCAACCATGCCAACTATTTAGGTAAGATTTTACTGTCTTTGATTGATATTAAATAC |
| | | TTAGCCGTGGCGTTATCAATGAATGATAATAAAAATGCGGATAAAAGCCAAAGGTGTTGG |
| | | AAATAAATCCAAGAATGAAGACGTAGATGTCGATGGGTATTTTAAGAACTTGAATTTGTC |
| | | ACGACTCACACGTTAAAATATATTATCCGAATTGTTTAGTCTAAAGACACACATATATTG |
| | | AAAAAGAAAAGGTAAATGAAGCTCATTGGTGCCTAAATGTGAAATGAAGCCGAAATGTGT |
| | | TAGGTGAACACATTTAAATATACAAAAAGAAATATAATAGAAACAAAACTAATTAACAAA |
| | | GTCGCAATTTGTATTGTATAAAATATCTTTCCGTCTCCCGTCATATTTGAAAAAAAAAAA |
| | | ATTACAAATCTGTTAATTTTAAAACTTTCTAGAAAAACACAAGTATATAATTTTCTCTTT |
| | | TCGTGCGTGTTTGTTTTAAAATAACATTGTTTTGATTGGCGACTCAACATATTTTAGCAT |
| | | TTACATATTTCTGCATATATTAAATGATTTATAAACTCAACTATAGATTAAAATATAATT |
| | | TGAGATCTAATAATTTTAACAATAATATAAAATATGAGATTTATAAATTAGGAATATAAA |
| | | TATTCAAGGGAGAGAAAAAGTAGAACATAATTCAAAAGATAAGACTTTTTAGACTTTTTT |
| | | AACAATATTTTTGATGGATAAAAATTATTCAAAAGAGAAGAAAGTAAGAAGAAAAGATGT |
| | | TTCTGAGAATT |
| 168 | DNA | Arabidopsis thaliana TBF1 promoter region with uORF1 and uORF2 |
| | | CGAGGAGTAGTTTACAGAGAATTTGGACCGTCCGATGTAAAGCGAAAATAGATCTAGGTT |
| | | TTCCACGTGTCCCCTATTTTAATGAAACCTTCTGATTCATGTAGAAGTTTTACTCAATTT |
| | | AATATTTTTTAGTATGTAGTTTTGTGTGTGTGTGTGTGTGTGTTTTTATGGCTCCACACC |
| | | AACTTTTAAAATGGTAGAAGCATGTTGCATGTGATCGAGTAAAAAGCCAATAATGAGATT |
| | | CAGAAAAATAAAAATTACTTATATAGTTTTTTAGAGAAAAAATTGTATTTTGTTTAAAGC |
| | | CTTAATCCGGTTGTTGAAAGAGCTGTGTCACGAGTTAAAAATATTTTCTTTTCATTTTTT |
| | | AAGTAATTAGTTTATAATGCAAAAATGGTTTTTATTTATTTGTCTTCGCTTATAGAACTG |
| | | CAAATTGAGAGAGAAAAAAATGAATTAGTGGTGGTGACCAAACATTCAGGAAGCTGTGAT |
| | | TGATCATTTGTTTTTGAGGTGAGTGTAGTGGCAACGTATGACGTTAACATATGGCGTACA |
| | | TAATAATTAGATGAACTTAATCATAATAATCATATTGCATTTAATTCATATATCATATCC |
| | | CATTAGTTGGACCACTTGATTTGAGGTCATGAGAAGAACATTTATGTTTTTTTTAGTTTG |
| | | AATCGGAGTGATCACTAAAAACTAGATACTGAAAATTTTCAAACTAAAATCATATTAATC |
| | | TTCAAAAAATGTGAAATCTAAAAAAAAAAAAAATTTTAACGCGTTCATTGTAGCCAAGTA |
| | | GCCAAGTATTGTTAAAGTAGTAGTAAAAGAAGTTTAGCTTTAAGTGATATAATTTGACAC |
| | | AAATCCTACTTAGATATGGATAATAGGATATAGCTTCATGTATATTTTTATCGTTGCTTC |
| | | TGTAACCCCAAAATGTGTTGATATAAGCATTTGAATATTCGTATGTATAATGTTTTCTTT |
| | | TCACCGTAAAACATATTACAATGTTAGTTTATATTGGATTTTGAATGTGTTTATGAACAG |
| | | TTTTTGTCGACTCAAAAGTTAAGATGAGAATATGGAAGAAAGTAAAGTTTAAAAGTCATG |
| | | ATGGGAACAAGGAATGGAACTCAAACATTCTAATACTCAACAAACGCAATTATATTATTA |
| | | CCATGACTCATCTTTCAAGTTCCATCAAAAAGATTCGTGGAAAATAATAGACTTACGTTT |
| | | CAAATCCATGTTTCTTTCTTTATAACAAAAAAAATGGATGTTTCTTGACGCGTGTCGAGA |
| | | GTACTCACCATTACTCTGACTTCAGTGAGTTTGGTCAAGTGGTCTTTTTTTTTCTCATGT |
| | | CACCAAAGGTCCAAACCCTAGAAATTAGTTCGAACTTTCCATAGAAGAACTGAATAAATG |
| | | GTCCAAAATTGTTTTAAAAAGGACCTAAGCCATTAGTTCATTGAATTCGAGTTAATGGGT |
| | | GAAGATTTTTATGATAACGAAAGTCGGAGTAATTATGCTTTTGGTCCGATAGTTTTCTAA |
| | | TTTGTTTTCTTTCCATTTTTTTTTTTTCAAATACTACATACTATATAAGATAGTGGTTTG |
| | | TGTTAATGTCATCGATGTGTTACCATCCGCATTATATTAATTATTTATCCCAACATAAAG |
| | | TCAGAATCTGTAATTTCTTTGTTATAAAATACAGTAAATGGTTCCGTTTAAGCTGTTAGA |
| | | TGATTTTTGAGTAAAAACTAATGTAAAAAAAACAAAAAAAAAACAATGTAGTTCATAATA |
| | | CATGCATGTTTTAAAGAAGTTTCTTGTTTACTATCAACTTGAATAGTATTTCACGAAGTC |
| | | AAAATTGTTCATTCCGACTTTTCTATGTGGAGAAAAAAAATTCTATCATTGTGCACAATT |
| | | TAACAGAATGTAATTTCTTGTAAAAGAAGAGGAAACAATTCGCTGTTAGTAAATGTGAAG |
| | | TATAGAAGTCTAAAATGAGATACCTCAACTAGCTTGAATTAAGAAAAAAAACAAAAACTC |
| | | TATCGACATGAAAAAGGTCGCAAATATTTATCATTTATCAATGCCAAAGGAGTATTTGGT |
| | | TCACAAAATACTGAATCATTTATATAGATATATAATTAGCTCTAAATTCTACTATAACTT |
| | | GCAAAATAAGTATACTGACTCAATTATATAGCGTTTAAAAATAGACGATTTGTATGATGA |
| | | GGTCCATATATATGGAGATGTGCATGCAACTATCGACATTTTCACACGTTGATATCGTCT |
| | | TTCTCCAATGGAGACTTGAATTTGTGTAAACTATGAATACTCGTCTCTCTAAGACCTTTT |
| | | TTCTTCAACCATGCCAACTATTTAGGTAAGATTTTACTGTCTTTGATTGATATTAAATAC |
| | | TTAGCCGTGGCGTTATCAATGAATGATAATAAAAATGCGGATAAAAGCCAAAGGTGTTGG |
| | | AAATAAATCCAAGAATGAAGACGTAGATGTCGATGGGTATTTTAAGAACTTGAATTTGTC |
| | | ACGACTCACACGTTAAAATATATTATCCGAATTGTTTAGTCTAAAGACACACATATATTG |
| | | AAAAAGAAAAGGTAAATGAAGCTCATTGGTGCCTAAATGTGAAATGAAGCCGAAATGTGT |
| | | TAGGTGAACACATTTAAATATACAAAAAGAAATATAATAGAAACAAAACTAATTAACAAA |
| | | GTCGCAATTTGTATTGTATAAAATATCTTTCCGTCTCCCGTCATATTTGAAAAAAAAAAA |
| | | ATTACAAATCTGTTAATTTTAAAACTTTCTAGAAAAACACAAGTATATAATTTTCTCTTT |
| | | TCGTGCGTGTTTGTTTTAAAATAACATTGTTTTGATTGGCGACTCAACATATTTTAGCAT |
| | | TTACATATTTCTGCATATATTAAATGATTTATAAACTCAACTATAGATTAAAATATAATT |
| | | TGACATCTAATAATTTTAACAATAATATAAAATATGAGATTTATAAATTAGGAATATAAA |
| | | TATTCAAGGGAGAGAAAAAGTAGAACATAATTCAAAAGATAAGACTTTTTAGACTTTTTT |
| | | AACAATATTTTTGATGGATAAAAATTATTCAAAAGAGAAGAAAGTAAGAAGAAAAGATGT |
| | | TTCTGAGAATTTCTAGAAACAGCATCCGTTTTTATAATTTAATTTTCTTACAAAGGTAGG |
| | | ACCAACATTTGTGATCTATAAATCTTCCTACTACGTTATATAGAGACCCTTCGACATAAC |
| | | ACTTAACTCGTTTATATATTTGTTTTACTTGTTTTGCACATACACACAAAAATAAAAAAG |
| | | ACTTTATATTTATTTACTTTTTAATCACACGGATTAGCTCCGGCGAAGTATGGTCGTCGT |
| | | CTTCATCTTCTTCCTCCATCATCAGATTTTTCCTTAAATGGAAGAAACCAAACGAAACTC |
| | | CGATCTTCTCCGTTCTCGTGTTTTCCTCTCTGGCTTTTATTGCTGGGATTGGGAATTTCT |
| | | CACCGCTCTCTTGCTTTTTAGTTGCTGATTCTTTTTCCTTCGACTTTCTATTTCCAATCT |
| | | TTCTTCTTCTCTTTGTGTATTAGATTATTTTTAGTTTTATTTTTCTGTGGTAAAATAAAA |
| | | AAAGTTCGCCGGAG |

### EXAMPLES

### Example 1

### Formation of NPR1 Condensates Promotes Cell Survival During Plant Immune Response

### Summary

In plants, pathogen effector-triggered immunity (ETI) often leads to programmed cell death, which is restricted by NPR1, an activator of systemic acquired resistance. However, the biochemical activities of NPR1 enabling it to both promote defense and restrict cell death remain unclear. Here we show that NPR1 promotes cell survival by targeting substrates for ubiquitination and degradation through formation of salicylic acid-induced NPR1 condensates (SINCs). SINCs are enriched in stress response proteins, including nucleotide-binding leucine-rich repeat immune receptors, oxidative and DNA damage response proteins, and protein quality control machineries. Transition of NPR1 into condensates is required for the formation of the NPR1-Cullin 3 E3 ligase complex to ubiquitinate SINC-localized substrates, such as EDS1 and specific WRKY transcription factors, and promote cell survival during ETI. Our analysis of SINCs suggests that NPR1 is centrally integrated into the cell death/survival decisions in plant immunity by modulating multiple stress-responsive processes in this quasi organelle.

### Introduction

Eukaryotes have evolved mechanisms to effectively restrict infection while avoiding significant damage to self. Such a regulation is evident during effector-triggered immunity (ETI) in plants, which is activated upon recognition of pathogen effectors by the nucleotide-binding and leucine-rich repeat immune receptors (NB-LRRs). ETI often culminates in rapid programmed cell death (PCD) at the site of infection to restrict pathogen growth. However, without a counteractive mechanism, ETI can severely compromise host survival due to the spread of cell death to uninfected tissues. One of the counteractive mechanisms is the systemic acquired resistance (SAR) mediated by salicylic acid (SA) through the function of NPR1 [non-expresser of pathogenesis related (PR) genes 1]. NPR1 is a master regulator of genes in multiple cellular machineries, including antimicrobial PR genes and endoplasmic reticulum-resident genes, that protect plants against a broad spectrum of diseases and stresses. In addition to NPR1-mediated transcription, the ubiquitin-proteasome system (UPS) and autophagy are sequentially recruited to maintain defense protein homeostasis and promote host survival. Mutants in the autophagy pathway are defective in the containment of age- and immunity-associated PCD due to proteotoxic accumulation of ubiquitinated proteins, which is partly dependent on SA and NPR1. Though early studies have implicated SA and NPR1 in suppressing ETI-induced cell death, the underlying mechanism is unknown.

In NPR1, the presence of a Broad-Complex, Tramtrack and Bric a brac (BTB) domain, in combination with a putative substrate-binding ankyrin-repeat domain, suggests that it may function as a Cullin 3 RING E3 ligase (CRL3) adaptor. However, an early yeast two-hybrid study failed to detect NPR1-CUL3 interaction. Instead, two NPR1 paralogs, NPR3 and NPR4, were later found to function as CRL3 adaptors in mediating NPR1 degradation in the nucleus.

Different activities of NPR1 are likely controlled by posttranslational modifications (PTMs), such as SUMOylation which is preceded by dephosphorylation and phosphorylation at two distinct IKB-like degrons, S55/59 and S11/15, respectively. SUMOylation not only regulates NPR1 nuclear-cytoplasmic partitioning and affects its association with different transcription factors (TFs), but also promotes its degradation by NPR3/4. Another important PTM triggered by SA is the oligomer-to-monomer transition of NPR1 through its conserved cysteines in response to cellular redox changes. Recent studies indicate that PTMs and changes in the cellular redox environment can lead to phase transition in many essential hub proteins enabling them to carry out diverse cellular functions.

In this study, we report that NPR1 contains intrinsically disordered regions (IDRs). In response to SA, NPR1 undergoes transition into cytoplasmic condensate-like structures enriched in proteins regulating ETI cell death, DNA damage response, redox metabolism, and ubiquitination. This SA-triggered NPR1 condensation is mediated through conserved cysteine clusters present within its IDRs and is required for the formation of a functional NPR1-CRL3 adaptor complex in the cytoplasm. Using cell biology, molecular and genetic analyses, we demonstrate that recruitment of the CRL3 ubiquitination machinery into SA-induced NPR1 condensates is an essential function of NPR1 in mediating protein homeostasis and cell survival. We provide evidence that the master immune regulator, EDS1, and TFs WRKY54 and WRKY70 are among the substrates of the NPR1-CRL3 complex for SA-mediated cell survival during ETI.

### Results

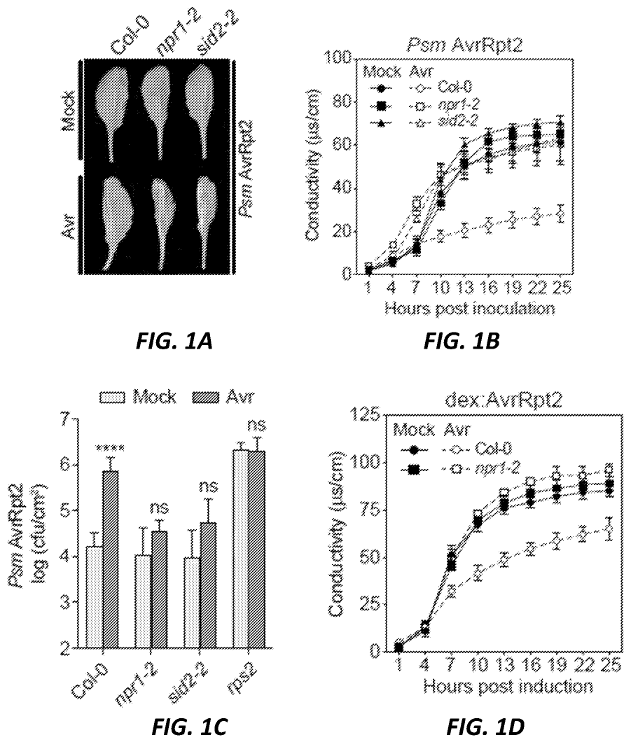

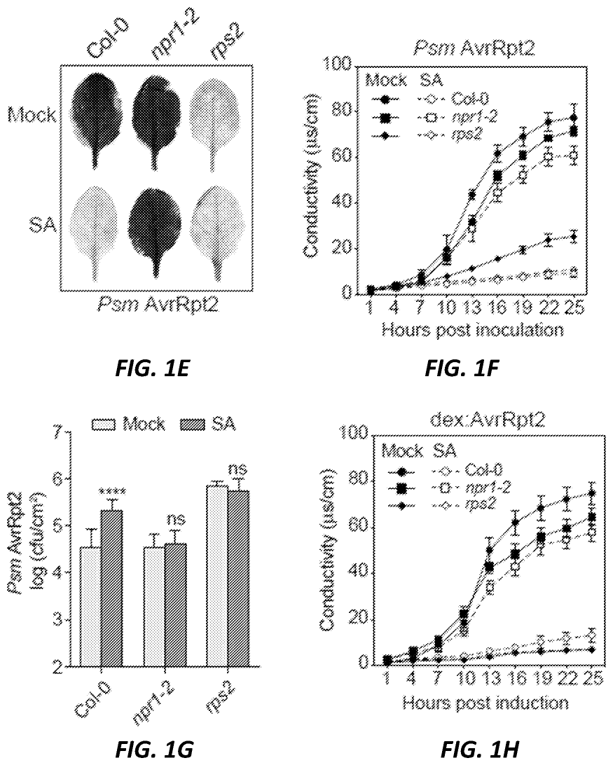

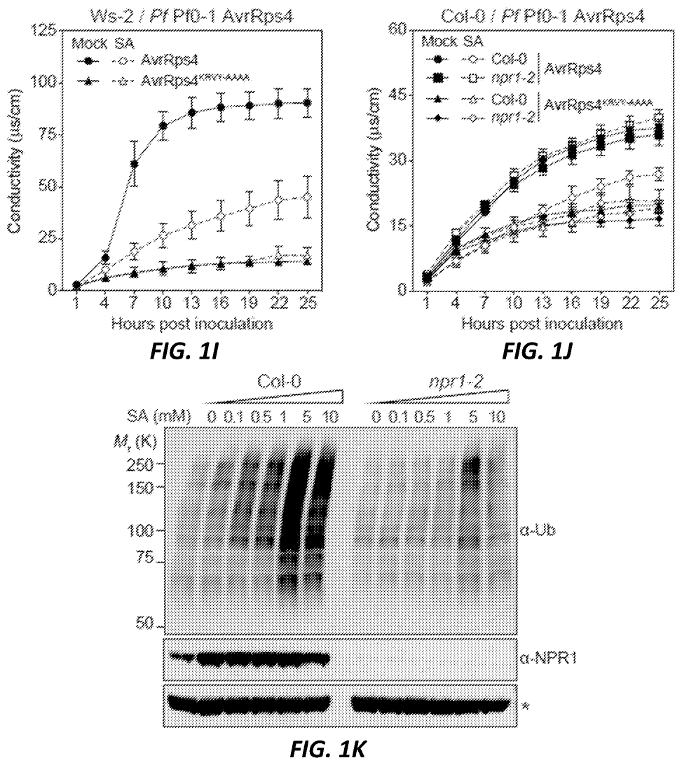

SA Promotes Cell Survival and Ubiquitination through the Function of NPR1. To elucidate the role of NPR1 in the regulation of cell survival, we first infected half leaves of Arabidopsis plants with Pseudomonas syringae pv. maculicola ES4326 carrying the AvrRpt2 effector (Psm ES4326/AvrRpt2) to systemically activate NPR1 and induce SAR. Then the other halves of the leaves were challenged with the same pathogen to determine how the ETI-associated cell death was affected in the neighboring tissue by measuring tissue collapse and conductivity increases due to electrolyte leakage (FIGS. 1A and 1B).

We found that in wild type (WT) plants, the first inoculation significantly suppressed PCD triggered by the second inoculation in the neighboring tissue. This suppression was SA- and NPR1-dependent because it was not observed in the SA biosynthesis mutant, sid2-2, or in the npr1-2 mutant. Consistent with our hypothesis that systemic activation of NPR1 inhibits ETI-induced cell death in the neighboring tissue, significant growth of the otherwise avirulent Psm ES4326/AvrRpt2, close to the level of the receptor mutant rps2, was observed in the second leaf halves in the WT plants, but not in npr1-2 or sid2-2 mutants (FIG. 1C).

To distinguish whether this SA- and NPR1-dependent cell survival occurs at the host cell level or is due to differential pathogen growth, we made use of an Arabidopsis line carrying the dexamethasone (dex)-inducible AvrRpt2 effector gene (dex:AvrRpt2) in either WT or the npr1-2 background. We found that in WT, induction by Psm ES4326/AvrRpt2 could protect plants against cell death triggered by subsequent in planta expression of AvrRpt2 in the neighboring tissue. This effect was abolished both in npr1-2 (FIG. 1D) and sid2-2 (FIG. 8A).

These results indicate that in pathogen-induced SAR, both SA and NPR1 are required at the host cell level for inhibiting ETI-induced PCD.

To determine whether SA is sufficient to inhibit ETI-triggered cell death, we pretreated plants with SA and found that at 0.5 and 1 mM, SA could fully suppress Psm ES4326/AvrRpt2-mediated cell death (FIGS. 8B, 1E and 1F), accompanied with increased pathogen growth (FIG. 1G), in WT plants, but not in the npr1-2 mutant. Similar to pathogen-induced SAR, SA treatment could protect against cell death triggered by in planta expression of the effector, AvrRpt2 (FIG. 1H). This SA/NPR1-mediated inhibition is not limited to ETI mediated by RPS2, which is a coiled-coil class of NB-LRR (CNL) immune receptor, but also ETI activated by RPS4 (FIGS. 1I and 1J) and RPP1 (FIG. 8C), which are Toll/Interleukin-1 receptor class of NB-LRRs (TNLs) against effectors AvrRps4 and ATR1, respectively. Taken together, these results indicate that SA plays a general role in promoting survival of neighboring cells during ETI through the function of NPR1.

To determine how SA-induced and NPR1-dependent cell survival correlates with protein homeostasis, we examined the accumulation of ubiquitinated proteins after treating WT and npr1-2 plants with SA. We found SA concentration-dependent accumulation of ubiquitinated proteins in WT, which is absent in npr1-2 (FIG. 1K). This result indicates that SA and NPR1 are involved in the accumulation of ubiquitinated proteins and implies that NPR1, considering its predicted structural features, might recruit the ubiquitination machinery and/or directly function as an E3 ligase adaptor in controlling protein homeostasis.

NPR1 Accumulates in the Cytoplasm and Undergoes SA-Triggered Condensate Formation. To determine the likely cellular compartment where NPR1-mediated ubiquitination occurs, we analyzed the subcellular accumulation of the endogenous NPR1 after SA treatment. As expected, NPR1 had predominant cytoplasmic accumulation in the mock-treated sample (0 mM SA), and an increased nuclear accumulation at 0.1 and 0.5 mM SA with corresponding decreases in the cytoplasmic fraction (FIG. 2A, upper panel).

Surprisingly, at 1 mM SA, more NPR1 accumulated in the cytoplasm, accompanied by its relative reduction in the nuclear fraction. With a longer exposure of the western blot, we could observe ubiquitinated NPR1 (higher MW smear) in the nuclear fractions of the SA-treated samples (FIG. 2A, middle panels). In the cytoplasmic fraction, however, no corresponding high MW NPR1 smear was observed, even though significant levels of the unmodified NPR1 and other ubiquitinated proteins were present (FIG. 2A, middle panels). A detailed comparison of protein bands between different SA concentrations showed a good correlation between levels of ubiquitination and the accumulation of NPR1 in both cytoplasm and nucleus (FIG. 8D). These results indicate that nuclear-cytoplasmic partitioning of the endogenous NPR1 is dynamically regulated by the concentrations of SA and the protein is unstable in the nucleus.

To validate the fractionation result, we monitored the nuclear-cytoplasmic partitioning of NPR1-GFP in Arabidopsis using transient expression assay. We observed significant levels of NPR1-GFP in the cytoplasm of untreated plants (FIG. 2B), similar to what was detected in the fractionation experiment with the endogenous protein (FIG. 2A). Treatment with 1 mM SA led to predominant nuclear accumulation of NPR1-GFP; whereas at 5 mM SA, fluorescence was detected also in the cytoplasm. This trend was consistent with the fractionation result, albeit higher SA concentrations had to be used for this imaging experiment. Surprisingly, SA also induced formation of discrete NPR1-GFP bodies, not only in the nucleus, but also in the cytoplasm (FIG. 2B). Importantly, cytoplasmic bodies were also observed at the lower SA concentration when we used the SUMOylation-deficient mutant of NPR1, npr1sim3 (sim3 henceforth), which has a predominant cytoplasmic localization after SA treatment (FIG. 2B). The latter observation indicates that body formation in the cytoplasm is not a result of possible toxicity of the higher SA concentration, but rather is a consequence of a specific PTM of NPR1, in this case, SUMOylation. Moreover, analysis of sim3-GFP localization in transgenic Arabidopsis showed that these bodies form only in the surviving cells adjacent to the ETI-induced cell death zone, ruling out a possible toxic effect of exogenous SA application (FIG. 8E). Interestingly, among immunity-associated paralogs (NPR1-4), only NPR1 had significant SA-inducible cytoplasmic body formation (FIGS. 9A-9C).

Moreover, development-associated NPRS and NPR6 showed constitutive bodies as previously reported. Bimolecular fluorescence complementation (BiFC) showed that NPR1 interactions with other NPRs are unlikely to trigger its cytoplasmic body formation (FIG. 9D). This NPR1-specific process may reflect its functional divergence from NPR2-4. These results reveal that besides dynamically controlling the nuclear-cytoplasmic partitioning of NPR1, SA also triggers formation of NPR1 bodies.

Because formation of NPR1 bodies is an induced process, and the morphology of the bodies is similar to that of phase separated proteins (Banani et al., 2017), we hypothesized that NPR1 undergoes conditional transition from soluble to condensed state, possibly through phase separation, to form the observed structures. To test our hypothesis, we performed single-cell time-lapse imaging of NPR1-GFP body formation (FIG. 2C). Quantification of total fluorescence from each body showed a gradual increase in the signal intensity over time as a result of their growth in size (FIG. 2D). Importantly, in contrast to intensity, the number of bodies started to decrease after 50 min as a result of their fusion (FIG. 2E). These behaviors are features of biomolecular condensates driven by IDRs or multivalent domain/motif interactions.

To determine whether NPR1 harbors IDRs, we used all available prediction algorithms and found cysteine-dependent or redox-sensitive IDRs (RDRs) to have the highest probability (FIG. 2F and Table 3). Remarkably, the size and number of RDRs in NPRs (i.e., NPR1-6) closely correlated with their ability to form cytoplasmic condensates, with NPR1 having the highest score and longest RDRs (FIGS. 9A and 9E). To validate the prediction result, we mutated the cysteine clusters of NPR1 within its three putative RDRs: rdr1 (npr1C150/155/156/160A); rdr2 (npr1C378/385/394A); and rdr3 (npr1C511/521/529A) (FIG. 2F). We found that unlike WT, both rdr1 and rdr2 formed cytoplasmic condensates in the absence of SA treatment (FIGS. 10A-10C), indicating that these cysteine clusters are required for keeping NPR1 out of state transition in uninduced cells.

In contrast, mutating RDR3 had no effect on protein state in untreated cells (FIGS. 10A-10C), but completely abolished the SA-induced condensation in both cytoplasm and nucleus (FIGS. 2G and 2H), indicating that the cysteines in RDR3 are required for triggering NPR1 state transition. Moreover, the lack of SA-induced condensation correlated with the reduced transcriptional co-activation function of rdr3 (FIG. 2I) even though the protein had increased nuclear accumulation compared to WT (FIG. 10D). Consistent with these results, random mutagenesis has found point mutations enriched in these conserved cysteines and RDRs, particularly in RDR3 (FIG. 2F and Table 4). Analysis of the SA-mediated survival phenotype of transgenic plants expressing the rdr mutants in the npr1-2 background revealed that consistent with their nuclear-cytoplasmic partitioning (FIG. 10D) and transcriptional co-activation function (FIGS. 10E and 10F), rdr1 had no protection against cell death induced by Psm ES4326/AvrRpt2 (FIG. 10G), while rdr2 displayed stronger protection (FIG. 10H). Importantly, despite its enhanced nuclear accumulation, the rdr3 mutant partially compromised the protection against cell death (FIG. 10I), supporting our hypothesis that NPR1 condensation is required for its function in promoting survival. These results indicate that SA may trigger conformational change in NPR1 through modulation of the conserved cysteines present within or in vicinity of its disordered regions, which in turn leads to protein condensation. Based on the above cellular and genetic data, we designate the NPR1 bodies as “SINCs” for SA-induced NPR1 condensates. Whether SINCs form through liquid-liquid phase separation will require further biophysical studies. Together, these results show that SA not only induces NPR1 transcriptional activity and turnover in the nucleus, but also triggers SINC formation in the cytoplasm, suggesting that SA regulates protein homeostasis and cell survival through well-coordinated nuclear and cytoplasmic functions of NPR1.

SINCs are Enriched with Stress Proteins and Ubiquitination Components. To uncover the function of cytoplasmic NPR1 condensates, we performed quantitative proteomic analysis using the cytoplasmic sim3 mutant. To ensure that SA-mediated transcription by the nuclear NPR1 is intact, the sim3-GFP was expressed in the WT NPR1 background (sim3-GFP/Col-0). After confirming that the line had proper SINC formation (FIG. 11A) and SA-induced ubiquitination (FIG. 11B), immunoprecipitation was performed against sim3-GFP in mock- and SA-treated plants followed by quantitative LC-MS analysis of co-purified proteins (FIG. 11C).

From these samples with good reproducibility (FIGS. 11D and 11E), we identified 171 proteins (“SINC components”) with statistically significant over two-fold enrichment in the SA-treated samples versus mock (Table 5). Gene Ontology (GO) term analysis revealed enrichment in defense/stress-associated proteins in Biological Process (FIG. 11F), protein modification and catabolism in Molecular Function (FIG. 11G), and cytosol in Cellular Component (FIG. 11H) categories. Based on the GO term analysis and literature search, the SINC components were assembled into six intersecting functional groups: (1) defense response, cell death and SA signaling; (2) protein homeostasis; (3) redox metabolism; (4) inter-organellar trafficking/protein transport; (5) DNA damage response; and (6) RNA binding/translation (FIG. 3A and Table 5).

FIG. 3B lists representatives of the four major functional groups. Notably, among the defense-related proteins, we found 10 NB-LRR disease resistance proteins together with other positive regulators of ETI, such as EDS1 (enhanced disease resistance 1) and PAD4 (phytoalexin deficient 4) (Heidrich et al., 2011); activators of hypersensitive cell death, such as BCS1 (cytochrome bc1 synthase 1); and proteins involved in SA-binding and signaling, such as NPR3. In the redox metabolism group, we found the cysteine-regulating glutathione pathway enzymes, such as glutathione peroxidase 8 (GPX8), glutathione S-transferase F6 (GSTF6) and glutathione S-transferase TAU 19 (GSTU19), which were previously identified in the SA-induced proteome. The second large group of proteins were associated with protein quality control, such as heat shock proteins, the ubiquitination machinery, such as ubiquitin, components of E1, E2, E3 ligase complex, and cysteine proteases, including ubiquitin-specific proteases (FIG. 3B and Table 5). Analysis of selected SINC components, EDS1 and BCS1 from the ETI/cell death group, and GSTU19 from the redox metabolism group, confirmed their full co-localization with sim3-GFP in the SA-induced condensates (FIG. 3C).

We then sought to determine the role of SA/NPR1 in the specific cellular pathways identified in SINCs, such as heat shock, oxidative and DNA damage responses, based on the hypothesis that over-accumulation of these stress-responsive proteins would result in cell death. Indeed, we found that SA significantly suppressed cell death induced by these stresses in WT, but not in the npr1-2 mutant plants (FIG. 3D-3F), supporting our hypothesis that NPR1 promotes cell survival in response to a broad spectrum of stimuli by sequestrating and/or degrading not only key immune regulators, but also other stress proteins in SINCs.

NPR1 recruits CUL3 to Cytoplasmic Condensates. The presence of ubiquitin, ubiquitin ligases and E3 ligase complex components in SINCs suggests that NPR1 may regulate protein homeostasis by recruiting ubiquitination machinery as a CRL3 adaptor. Previous studies have shown that members of the NPR family can associate with CUL3 and serve as adaptors for ubiquitination of cognate substrates.

To test our hypothesis, NPR1 and CUL3 were co-expressed in Nicotiana benthamiana followed by 1 mM SA treatment and co-immunoprecipitation (co-IP). We found that the WT NPR1 had a weak pull-down signal of CUL3 in the SA-treated sample (FIG. 4A).

A similar interaction was also detected with the endogenous CUL3 in Arabidopsis (FIG. 4B). Interestingly, when co-expressed in E. coli, an interaction between the two proteins could be easily observed (FIG. 4C), suggesting that NPR1-CUL3 association might be inhibited in planta by PTMs that are absent in E. coli.

To identify regions and residues that could influence CUL3-NPR1 interaction, we included different truncations and point mutants of NPR1 in our co-IP assay (FIG. 12A).

At the lower concentration of 1 mM SA used in this screen, the WT NPR1 is predominantly nuclear localized (FIG. 12B), allowing us to identify PTMs that could enhance the interaction with CUL3, assuming that it occurs in the cytoplasm. First, we found that unlike the full-length protein, the BTB domain of NPR1 was sufficient to strongly pulldown CUL3 even in the absence of SA (FIGS. 4A and 12C), consistent with the notion that BTB is the CUL3-interacting domain in CRL3 substrate adaptors. The BTB domain was also required for interacting with the endogenous CUL3 in Arabidopsis (FIG. 4B) and in E. coli (FIG. 4C). Similarly, deleting the C-terminal domain (CTD) of NPR1 in ACTD significantly enhanced the interaction with CUL3 in the absence of SA (FIGS. 4A and 12C). Moreover, the CTD interacted with the BTB domain and outcompeted CUL3 for interaction with BTB when co-expressed (FIG. 4D). These data suggest that the full-length NPR1 is inhibited from interacting with CUL3 by its CTD. Among point mutants with enhanced cytoplasmic localization, sim3 and nls showed significantly stronger CUL3 pull-down signals compared to the WT NPR1, while other such mutants, S55/59D and npr1-3, had very little signal (FIGS. 4A, 4B, and 12C). These data indicate that NPR1-CUL3 interaction is controlled by NPR1 PTMs, such as SUMOylation and phosphorylation at S55/59, which affect NPR1 nuclear-cytoplasmic partitioning and, perhaps, SINC formation.

To test this hypothesis, we used the BiFC assay to examine the subcellular localization of NPR1-CUL3 interaction. Both NPR1 and sim3 showed clear interactions with CUL3 in discrete cytoplasmic condensates, with the signal more intense for sim3 (FIGS. 4E and 4F). In contrast, the phosphomimic mutant, S55/59D, showed a significantly weaker BiFC signal. The reduced S55/59D interaction with CUL3 observed in the pull-down and the BiFC assays (FIGS. 4A, 4E and 4F) correlated with its inability to form SINCs (FIGS. 4G and 12B). A correlation was also observed for the stronger CUL3 interactors, sim3, which could form condensates at a lower concentration of SA (FIG. 12B), and ACTD, which formed constitutive condensates (FIG. 12D). Unlike NPR1, NPR2-4 interactions with CUL3 were predominantly in the nucleus (FIGS. 13A and 13B).

In further support of our hypothesis that the NPR1-CUL3 interaction occurs in cytoplasmic condensates, rdr1 and rdr2, which constitutively form condensates, showed strong interactions with CUL3, whereas rdr3, which is deficient in condensate formation, displayed little interaction (FIG. 4H).

To demonstrate that it is NPR1 that recruits CUL3 to the condensate instead of CUL3 engaging NPR1 to a pre-existing complex, we analyzed GFP-CUL3 localization in the NbNPR1 knockdown plants (FIGS. 13C and 13D). We found that compared to control, silencing of NbNPR1 reduced the SA-induced accumulation of GFP-CUL3 in cytoplasmic condensates, even though similar levels of the GFP-CUL3 were present (FIGS. 4I-4L). Together, these results show that NPR1 recruits CUL3 into SINCs through its BTB domain.

NPR1-CUL3 Condensates Are Active Ubiquitination Complexes. To determine whether recruitment of CUL3 into SINCs is associated with increased ubiquitination activity, we first examined the co-localization of NPR1-CUL3 complex with known markers of protein bodies involved in protein homeostasis: ubiquitin, ATG8, NBR1 (a polyubiquitin receptor) and HSC70. Because the sim3 mutant showed a stronger interaction with CUL3, we used the sim3/CUL3 BiFC pair for this analysis. We found that sim3/CUL3 condensates co-localized strongly with all four markers of protein bodies, but not with markers of the morphologically similar membrane-bound bodies such as Golgi, TGN and MVB (FIGS. 5A and 13E).

Importantly, co-localization of sim3-GFP with mCherry-NBR1, was significantly enhanced by the presence of CUL3 (FIG. 5B), suggesting that the recruitment of a functional CUL3 into the NPR1 condensates is required for the accumulation of polyubiquitinated protein species in these structures.

To further establish the correlation between the SA-dependent recruitment of CUL3 into condensates and protein ubiquitination, we performed NPR1-CUL3 and sim3-CUL3 pull down under dose response to SA. We found an increase in the interaction between 0-1 mM SA for both NPR1 and sim3 (FIG. 5C, upper panel). By testing the level of total protein ubiquitination in the same samples, we observed a clear correlation between formation of the NPR1-CUL3 complex and the accumulation of ubiquitinated proteins (FIGS. 5C, lower panel, and 13F). Notably, the baseline SA-dependent ubiquitination was lower for sim3 than NPR1, possibly because sim3 is defective in its nuclear function in inducing transcription of potential substrates and/or ubiquitination machinery components. Interestingly, the reduction in ubiquitination at 3 mM SA correlated with reduced neddylation of CUL3 (FIG. 5C, upper Myc-CUL3 band), suggesting that ubiquitination observed in this experiment was a result of active CUL3. To confirm that this NPR1-mediated ubiquitination is dependent on CUL3 activity, we created an inactive, neddylation-deficient form of CUL3 (CUL3ARBX1) and observed a reduction in its ubiquitination activity (FIG. 5D). A similar reduction was observed when we silenced the endogenous N. benthamiana CUL3 (NbCUL3) (FIGS. 5E, 13C and 13D). Together, these results indicate that recruitment of CUL3 into SINCs is associated with increased ubiquitination activity, and further support our hypothesis that SINCs and SINC components can be targeted for degradation by the cytoplasmic NPR1-CRL3 complex to promote cell survival.

NPR1 Targets SINC-localized Proteins for Ubiquitination and Degradation. Many SINC proteins identified in our proteomic analysis, such as NB-LRRs and EDS1/PAD4 involved in ETI signaling (FIG. 3; Table 5), are prime candidates for explaining the role of NPR1 condensates in preventing ETI-induced cell death (FIGS. 1A-1K). The presence of diverse stress-responsive proteins in the NPR1 condensates is consistent with the central role that NPR1 plays in promoting cell survival under a broad range of adverse conditions that could lead to cell death (FIGS. 3A-3F). We selected EDS1 to test our hypothesis. First, we found that EDS1 could interact both with WT and cytoplasmic sim3 mutant in planta (FIG. 6A) and in E. coli (FIG. 6B), indicating that NPR1 recruits EDS1 into SINCs through a direct interaction.

Next, we observed complete co-localization of EDS1 with NPR1/CUL3 and sim3/CUL3 bodies (FIG. 6C) suggesting that NPR1 may target EDS1 for degradation in SINCs through NPR1-CRL3-mediated ubiquitination. To test this hypothesis, we examined the stability of EDS1 in the WT and npr1-2 plants in the presence of SA and protein synthesis inhibitor cycloheximide (CHX). We found that in WT plants, the amount of EDS1 significantly decreased in the absence of new protein synthesis, and this decrease was dependent on SA-mediated activation of NPR1 (FIGS. 6D and 14A).

In npr1-2, while the accumulation of EDS1 was low, the protein levels remained unchanged after SA treatment. Similar results were obtained with another SINC-localized and NPR1-dependent and interacting protein, NIM1-interacting 1 (NIMIN1) (FIGS. 14A and 14B). These results indicate that specific immune regulators directly interacting with NPR1 could be targeted for degradation in SINCs through NPR1-CRL3-mediated ubiquitination. Indeed, we observed increased SA-mediated ubiquitination of EDS1 in WT, but not in npr1-2 nor eds1-2 which is a mutant of one of the two homologous EDS1 genes in Col-0 (FIG. 6E, upper blot). Interestingly, eds1-2 mutant showed reduced levels of SA-induced ubiquitination (FIG. 6E, bottom blot), consistent with the requirement of EDS1 for amplification of SA signaling in plants. Together, these results support our hypothesis that NPR1 targets EDS1 to promote survival during ETI.

WRKY TFs Required for ETI Are Targets of the NPR1-CRL3 Complex. Because EDS1 is a major upstream immune regulator involved in not only ETI, but also SA synthesis, it would be difficult to use genetic epistasis to test the specific effect of NPR1 in recruiting EDS1 to SINCs for ubiquitination and degradation. To remedy this problem, we sought other potential candidates. Among the known NPR1-interacting proteins, WRKY70 TF has been shown to play opposing roles as a repressor of SA synthesis and SA-responsive genes and a positive regulator of various ETI. Like EDS1, WRKY70 interacts more strongly with the cytoplasmic sim3 mutant than the WT NPR1, suggesting that this WRKY TF and perhaps its close homolog WRKY54 may be recruited to SINCs. In support of this hypothesis, we found that these WRKYs could indeed co-localize with sim3/CUL3 bodies (FIG. 6F).

Moreover, analysis of the constitutively expressed WRKY70-GFP in WT and npr1-2 plants showed that while NPR1 was required for stabilization of WRKY70-GFP in the absence of SA, the protein level was reduced in an NPR1- and proteasome-dependent manner in the presence of SA (FIG. 6G). Consistently, we observed increased ubiquitination of WRKY70-GFP in the SA-treated WT but not in the npr1-2 mutant (FIG. 6H). In addition to the in vivo data, we reconstituted the NPR1-CRL3-mediated ubiquitination cascade in E. coli and showed that both WT NPR1 and the sim3 could mediate ubiquitination of WRKY70 (FIGS. 6I, 14C and 14D).

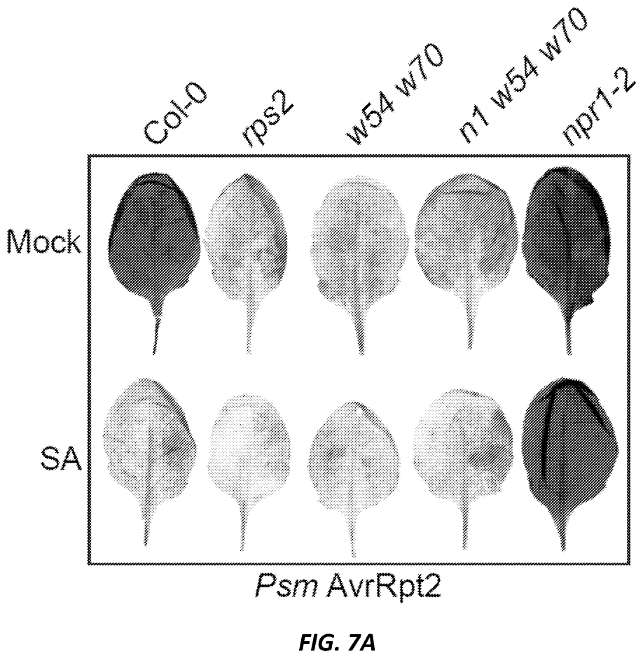

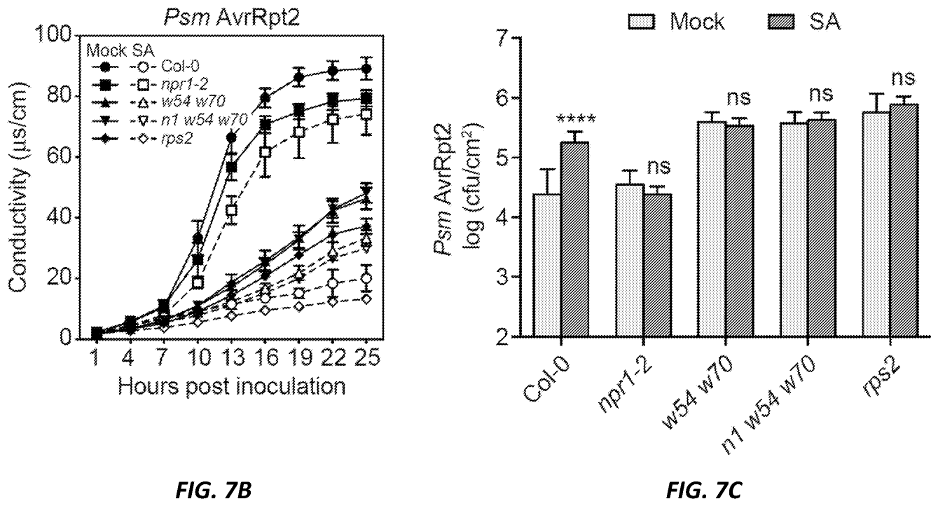

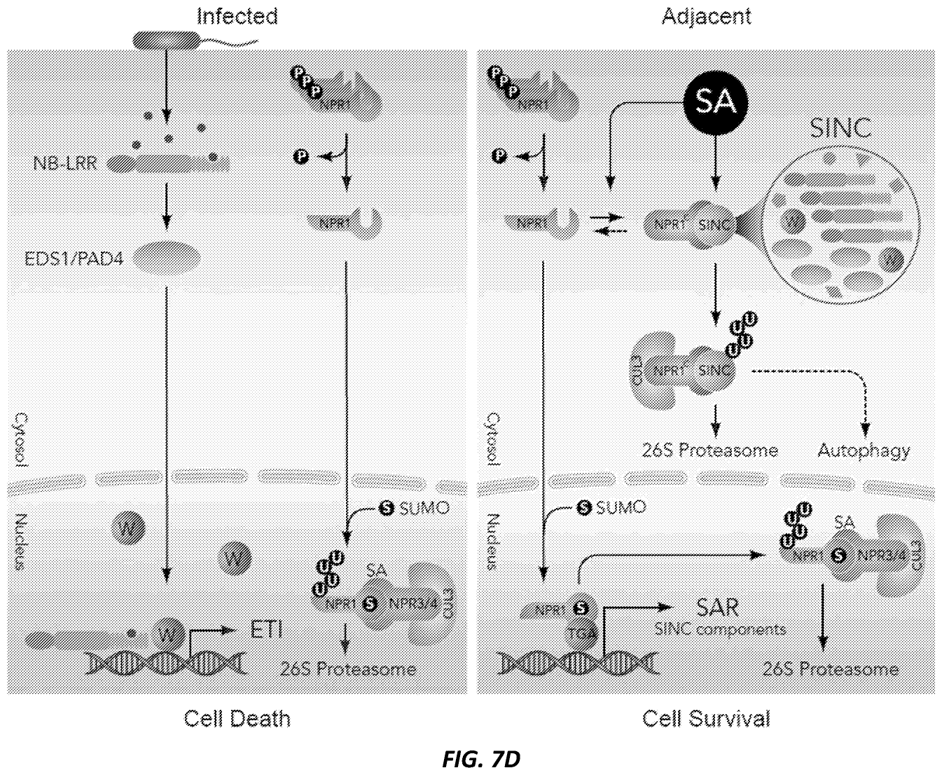

We next sought to put NPR1-mediated degradation of these WRKY TFs in the context of NPR1's function in promoting cell survival by testing ETI induced by Psm ES4326/AvrRpt2 in wrky54 wrky70 double and npr1 wrky54 wrky70 triple mutants. Mutating WRKY54 and WRKY70 diminished the ETI-triggered cell death (FIGS. 7A and 7B) and led to enhanced pathogen growth (FIG. 7C) independent of SA pre-treatment, confirming the positive role of these TFs in ETI.

Moreover, these wrky54 wrky70 phenotypes were epistatic to those of npr1-2 in the npr1 wrky54 wrky70 triple mutant, once again rendering strong support for our hypothesis that NPR1 inhibits cell death by degrading positive regulators of ETI such as WRKY54 and WRKY70. Based on the molecular, cellular, and genetic data, we present the following model to describe the molecular function of NPR1 in mediating cell survival (FIG. 7D). During ETI, infected cells turn on their cell death program as a result of NB-LRR activation by the pathogen effector and signal transduction through components such as EDS1/PAD4 and WRKY54/70 TFs. Concurrently, an increase in SA leads to dephosphorylation of NPR1 at S55/59, releasing NPR1 monomers to enter the nucleus. SUMOylation of NPR1 in the nucleus is not only required for its transcription cofactor activity, but also facilitates its degradation by the NPR3/4-CRL3 complex to remove its inhibitory effect on ETI. In adjacent cells, where ETI becomes unnecessary due to the low pathogen load, the cell survival program becomes predominant through SA-mediated activation of nuclear NPR1 to induce transcription of SAR genes, including SINC components, and the formation of SINCs in the cytoplasm to sequester and degrade proteins involved in cell death, such as NB-LRRs, EDS1, and WRKY54/70. Even though the tipping point of this cell death and survival decision is unknown, it is likely to involve PTMs of NPR1 regulated by the level of the pathogen signal. If the cell survival program is activated by treating plants with SA prior to being exposed to an ETI-inducing signal, they become deficient in ETI (FIGS. 7A-7C).

### Discussion

In this study, we found that NPR1 has a major role in controlling protein homeostasis through formation of previously unknown subcellular structures, SINCs, to sequester distinct stress-responsive components in the cytoplasm. Importantly, this cytoplasmic function of NPR1 likely occurs subsequent to its nuclear function in SA/NPR1-mediated transcriptional reprogramming, explaining why such a function was obscured in previous studies. Indeed, many of the proteins found in SINCs are SA-inducible (FIGS. 3A-3F; Table 5). Moreover, functional validation of the pathways identified in SINCs showed that NPR1 promotes survival in response to not only pathogen-induced cell death, but also to heat shock, oxidative and DNA damage responses (FIGS. 3A-3F). Importantly, many of the defense-associated SINC components are also part of ubiquitination machinery, such as E1, E2 and Cullin-associated NEDD8-dissociated protein 1 (CAND1) (FIG. 3B and Table 5). Because the great majority of SA-regulated transcriptome is NPR1-dependent without SA/NPR1-mediated transcriptional induction of defense genes and protein quality control machineries, it would not be necessary, nor possible to maintain protein homeostasis. For example, NPR1 not only induces NIMIN1, WRKY54 and WRKY70 gene transcription in the nucleus, but also mediates degradation of these TFs in the cytoplasmic SINCs. Therefore, mutants blocking the nuclear function of NPR1, such as sim3 and nls, are compromised in SAR even though they still maintain their abilities to interact with CUL3 and ubiquitinate substrates. By applying SA to first activate NPR1 condensate formation, we were able to observe the striking effect that NPR1 has on cell survival in the subsequent ETI tests using a high pathogen inoculant at which SA-mediated SAR is insufficient in rendering resistance (FIGS. 1A-1K and 7A-7D).

The dynamic distribution of NPR1 between nucleus and cytoplasm induced by SA allows coordination of NPR1′s function between the two compartments to achieve proper control of plant immune responses. This process is regulated at multiple steps: Pathogen-induced SA increase is known to change the cellular redox state, leading to the release of NPR1 monomer from the homo-oligomer to translocate into the nucleus. In the absence of SA, the NPR1 homo-oligomer does not form cytoplasmic condensates, nor interact with CUL3, probably due to phosphorylation at S55/59, because the phosphomimic mutant, S55/59D, is defective in both of these processes. SA-induced dephosphorylation at these two residues is required for activation of NPR1 to either enter the nucleus or to form SINCs in the cytoplasm as the phospho-deficient mutant S55/59A has autoimmunity and is severely retarded in growth. Dephosphorylation at S55/59 is also a pre-requisite for SUMOylation in the nucleus, because S55/59D is incompetent for this PTM (Saleh et al., 2015). SUMOylation and subsequent ubiquitination and degradation of nuclear NPR1 mediated by NPR3/4-CRL3 also interplay with SINC formation in the cytoplasm, as shown by the increased SINC formation in sim3 (FIG. 2B) and the sequestration of NPR3 in SINCs (FIG. 3B and Table 5).

Importantly, at all SA concentrations tested, the endogenous NPR1 was never completely depleted from the cytoplasm. In fact, we observed a clear increase in the levels of NPR1 protein in the cytoplasm at higher SA concentrations which correlated with protein condensation. However, we cannot rule out the presence of NPR1 condensates at lower SA concentrations because of the limitation in detection. Though high protein levels are required for visualizing condensates through microscopy, their formation is determined by the biological properties of the proteins. The phosphomimic S55/59D mutant and paralogs, NPR2, NPR3 and NPR4, all fail to form cytoplasmic condensates (FIGS. 4A-4L and 9A-9E), whereas NPR1's ability to form condensates is dependent on the specific redox-sensitive cysteine clusters within the predicted RDRs of the protein. A similar regulation was found in the E. coli heat shock protein 33 (Hsp33). This redox-sensing chaperone can form active bodies upon oxidative stress, which induces transition to a more disordered state, exposing the substrate-binding surface of the protein. Thus, SA-dependent transition of NPR1 into condensates could be triggered by its ability to sense the redox state of the cell, which is consistent with the enriched accumulation of glutathione pathway components in SINCs (FIGS. 3A-3F and Table 5). Formation of NPR1 condensates is required for its ability to recruit CUL3 (FIGS. 4A-4L). Accumulation of multiple interactors of NPR1 may facilitate its condensation and subsequent recruitment of CUL3. Indeed, for the well-studied CRL3 adaptor Speckle-type POZ protein (SPOP), substrate binding is necessary for its phase separation and formation of an active E3 ligase complex with CUL3.

Condensate formation is a feature of proteins that occupy essential hub positions in chromatin organization, transcription, translation, maintenance of cell architecture and protein quality control. Recent development in NPR1 research expanded the list of its interactors and cellular processes in histone modification, cold acclimation, unfolded protein response and SAR. Carrying out these functions would require formation of multi-protein complexes with diverse signaling and metabolic activities. The intrinsic ability of NPR1 to transition from one conformational state to another by forming condensates is a remarkable adaptation enabling it to regulate complex cellular processes, such as signal transduction and protein homeostasis under stress, to promote host survival. This may explain why overexpressing Arabidopsis NPR1 in heterologous plant backgrounds proved to be effective in engineering broad-spectrum disease resistance. Besides serving as a hub for regulating protein homeostasis during cell stress, SINCs may also play a signaling role in plant immunity. The presence of 10 NB-LRR immune receptors, their downstream components EDS1/PAD4, cysteine proteases, cellular redox regulators, ubiquitination, and DNA damage response proteins opens new areas of inquiry for possible interplay between these processes inside SINCs to uncover novel signaling mechanisms.

### Experimental Model and Subject Details

Arabidopsis thaliana (At) wild type (WT), mutants, and transgenic plants used in this study were all in the Col-0 ecotype background, with the exception of the Ws-2 ecotype which was used for Pseudomonas fluorescens (Pf) Pf0-1 AvrRps4 infection. Unless otherwise indicated, transgenic Arabidopsis over-expressing GFP-fused NPR1 or its mutant/truncation variants, are all in the npr1-2 mutant background. Arabidopsis mutants npr1-2, sid2-2, rps2, wrky54 wrky70 double and eds1-2; and transgenic lines over-expressing NPR1-GFP and sim3-GFP in the npr1-2 background, the dex:AvrRpt2/rps2 line, and the estradiol-inducible AvrRpt2 (est:AvrRpt2) lines in the Col-0, sid2-2 and rps2 backgrounds were described previously. Transgenic Arabidopsis over-expressing sim3-GFP in the Col-0 background, ABTB-GFP and rdr1/2/3-GFP in the npr1-2 background, and WRKY70-GFP in the Col-0 background, were generated and plants homozygous for the transgenes were used. Transgenic Arabidopsis over-expressing WRKY70-GFP in the npr1-2 background was generated by crossing WRKY70-GFP/Col-0 with the npr1-2 mutant. The dexamethasone-inducible AvrRpt2 line in the npr1-2 mutant background (dex:AvrRpt2/npr1-2) was generated by crossing dex:AvrRpt2/Col-0 (McNellis et al., 1998) with the npr1-2 mutant. The npr1 wrky54 wrky70 triple mutant was generated by crossing wrky54 wrky70 double mutant with npr1-2. Seeds were stratified at 4° C. for three days and plants were grown under a 12 hr light and 12 hr dark cycle at 22° C. Nicotiana benthamiana WT plants were grown under the same conditions. Unless otherwise indicated, in all experiments, soil-grown Arabidopsis and N. benthamiana plants were used at three-week-old and four-week-old age, respectively.

### Method Details

Plasmid construction and E. coli-based ubiquitination: The coding sequences for all Arabidopsis genes were amplified from cDNA. Point mutations of AtNPR1 (AT1G64280) were generated using the QuikChange II site-directed mutagenesis kit (Agilent). Overlap PCR was used to generate the deletion/truncation mutations of AtCUL3A (AT1G26830) and AtNPR1. The position of NPR1 truncations and point mutations are indicated in FIG. 12A and Table 4. The WT and mutated coding sequences for all genes and gene fusions were sub-cloned into the pDONR207 gateway donor vector and confirmed by sequencing. The obtained entry vectors were recombined into either plant or E. coli destination vectors without or with an N- or C-terminal tag. For in planta expression, genes were recombined into the plant binary vectors pK7FWG2, pSITE-4NB and pLN462 to generate C-terminal eGFP, mCherry and HA fusions, respectively; into pK7WGF2, pEG201, pEG202, pEG203 and pEG204 to generate N-terminal eGFP, HA, FLAG, Myc and V5 fusions, respectively. For co-expression of NPR1 and CUL3 in E. coli, NPR1 or its mutant/truncation variants were first recombined into pDEST15 vector to generate N-terminal GST fusions (GST-NPR1). Next, E. coli-codon-optimized coding sequence of AtCUL3A fused to the Myc tag was amplified from pEG203-CUL3 and inserted between NdeI and XhoI in the MCS-II of pCDFDuet-1 to generate pCDFDuet-1:Myc-CUL3 plasmid. The two plasmids, pDEST15-NPR1 and pCDFDuet-1:Myc-CUL3, were co-transformed into the E. coli strain BL21(DE3) and protein expression was induced with 0.25 mM IPTG for 12 hr at 20° C. For co-expression of NPR1 and EDS1 in E. coli, the coding sequence of AtEDS1 (AT3G48090) fused to FLAG tag was inserted between NcoI and NotI in MCS-I of pETDuet-1 to first generate the pETDuet-1:FLAG-EDS1 plasmid. Next, the coding sequences of GST-NPR1 fusion or GST alone were amplified from the corresponding pDEST15-based constructs and inserted between NdeI and AvrII in MCS-II of pETDuet-1:FLAG-EDS1 to generate pETDuet-1:FLAG-EDS1+GST-NPR1 or pETDuet-1:FLAG-EDS1+GST plasmid. The obtained plasmids were transformed into the E. coli strain BL21(DE3) and protein expression was induced with 0.25 mM IPTG for 12 hr at 20° C. The neddylation-deficient CUL3□RBX1 mutant was generated by deleting the RBX1 binding motif (F563-E581) in AtCUL3A based on the corresponding deletion in the HsCUL3 (Furukawa et al., 2003). mCherry-fused organelle markers were described previously: TGN (trans-Golgi network/early endosome), MVB (multivehicular body/late endosome); Golgi and Peroxisome; and HSC70 (heat shock cognate 70). The N-terminal mCherry-fusions of AtUbiquitin (AT5G03240), ATG8 (autophagy-related 8a; AT4G21980) and polyubiquitin receptor NBR1 (next to BRCA1 gene 1; AT4G24690) were generated by recombining entry vectors carrying the mCherry-fused coding sequences into the pEG100 plant binary vector. The C-terminal mCherry fusions of AtEDS1, AtGSTU19 (AT1G78380), AtBCS1 (AT3G50930), AtNIMIN1 (AT1G02450), AtWRKY54 (AT2G40750) and AtWRKY70 (AT3G56400) were generated by recombining entry vectors carrying the coding sequences into the pSITE-4NB plant binary vector. The mCherry-NLS nuclear marker was generated by recombining entry vector carrying the coding sequence of mCherry fused to the SV40 nuclear localization signal (CGGGPKKKRKVED (SEQ ID NO: 161)) into the pEG100 plant binary vector. For bimolecular fluorescence complementation (BiFC), entry vectors carrying the coding sequences of genes and their mutant variants were recombined into the pSITE-cEYFP-N1 binary vector for C-terminal fusion with the cYFP half (YC), or into the pSITE-nEYFP-C1 for N-terminal fusion with the nYFP half (YN). To construct the PR1 promoter reporter, entry vector carrying the 2367-bp upstream fragment of AtPR1 (AT2G14610) gene was recombined into a dual luciferase reporter system adapted for Gateway cloning, to generate the pPR1:DUAL-LUC (pPR1:FLUC/35S:RLUC) plant binary vector.

To generate the RNAi silencing vectors for NbNPR1 and NbCUL3, protein sequences for all six AtNPRs [AtNPR1, AtNPR2 (AT4G26120), AtNPR3 (AT5G45110), AtNPR4 (AT4G19660), AtNPR5 (AT2G41370) and AtNPR6 (AT3G57130)] and all six AtCULLINs [AtCUL1 (AT4G02570), AtCUL2 (AT1G02980), AtCUL3A (AT1G26830), AtCUL3B (AT1G69670), AtCUL4 (AT5G46210) and AtCUL5 (AT4g12100)] were used to retrieve orthologs in the N. benthamiana genome assembly (Fernandez-Pozo et al., 2015a). The coding sequences of two genes found in the NPR1 clade [Niben101Scf14780g01001.1 (NbNPR1a) and Niben101Scf11512g01004.1 (NbNPR1b)], and four genes found in the CUL3 clade [Niben101Scf01326g05017.1 (NbCUL3Aa), Niben101Scf05060g00001.1 (NbCUL3Ab), Niben101Scf06545g03015.1 (NbCUL3Ba) and Niben101Scf00272g10015.1 (NbCUL3Bb)] were further submitted to the virus-induced gene silencing (VIGS) tool to predict conserved gene fragments for silencing each clade (Fernandez-Pozo et al., 2015b). A 300-bp fragment was designed to silence the two NbNPR1 genes, and a 600-bp fusion fragment was designed to silence the four NbCUL3 genes. The fragments were amplified from N. benthamiana genomic DNA using gene-specific primers (Table 6) and cloned into the pTRV2-LIC plasmid to generate pTRV2-NbNPR1 and pTRV2-NbCUL3 plant binary vectors. The control vector carrying the N. benthamiana phytoene desaturase gene (pTRV2-NbPDS) was described previously. The ubiquitination reaction was carried out according to the previously described principle of reconstituting basic ubiquitination cascade in E. coli. To reconstitute the complex, CUL3-mediated ubiquitination cascade, the reaction components consisting total of seven proteins (Substrate, NPR1, CUL3, RBX1, E1, E2 and Ubiquitin) were co-expressed in E. coli using a modified Duet vector system (Novagen). Three expression vectors were constructed: (1) pETDuet-1:FLAG-WRKY70+GST-NPR1 or pETDuet-1:FLAG-WRKY70+GST-sim3 or pETDuet-1:FLAG-WRKY70+GST (pET-AdS); (2) pACYCDuet-1:RBX1+Myc-CUL3 (pACYC-RC3); and (3) pCDFDuet-1:HA-Ub+UBC8+UBA1 (pCDF-Ub; FIG. 14C). To construct pET-AdS, the coding sequence of AtWRKY70 fused to the FLAG tag was amplified from pEG202-WRKY70 and inserted between NcoI and NotI in MCS-I of the pETDuet-1 to generate the pETDuet-1:FLAG-WRKY70 plasmid. Next, the coding sequences of NPR1 or sim3 fused to GST, or GST alone, were amplified from the corresponding pDEST15-based constructs and inserted between NdeI and AvrII in MCS-II of the pETDuet-1:FLAG-WRKY70 to generate pETDuet-1:FLAG-WRKY70+GST-NPR1, pETDuet-1:FLAG-WRKY70+GST-sim3 or pETDuet-1:FLAG-WRKY70+GST plasmids. To construct pACYC-RC3, the E. coli-codon-optimized sequence of AtRBX1 (AT5G20570) was inserted between NcoI and HindIII in the MCS-I of pACYCDuet-1 to generate pACYCDuet-1:RBX1. Next, E. coli-codon-optimized sequence of AtCUL3A fused to the Myc tag was amplified from pEG203-CUL3 and inserted between NdeI and XhoI in the MCS-II of pACYCDuet-1:RBX1 to generate the pACYCDuet-1:RBX1+Myc-CUL3 plasmid. To construct pCDF-Ub, the coding sequence of AtUbiquitin (Ub) fused to HA was amplified from pEG201-Ub and inserted between NcoI and EcoRI in the MCS-I of pCDFDuet-1 to generate pCDFDuet-1:HA-Ub. Next, coding sequence of AtUBC8 (AT5G41700), in which its single NcoI restriction site was eliminated by introducing a silent mutation, was inserted between NcoI and HindIII in the MCS-I of pCDFDuet-1 to generate pCDFDuet-1:UBC8. Next, the entire fragment required for expression of UBC8 (T7 promoter, lac operator, rbs and UBC8) was amplified from pCDFDuet-1:UBC8 and inserted between EcoRI and NotI in the MCS-I of pCDFDuet-1:HA-Ub, downstream to HA-Ub, to generate the pCDFDuet-1:HA-Ub+UBC8 plasmid. Next, coding sequence of AtUBA1 (AT2G30110) was inserted between FseI and AvrII in the MCS-II of pCDFDuet-1:HA-Ub+UBC8 to generate the pCDFDuet-1:HA-Ub+UBC8+UBA1 plasmid. The three vectors, pET-AdS, pACYC-RC3 and pCDF-Ub, were co-transformed into the E. coli strain BL21(DE3) and ubiquitination reaction was initiated by inducing protein expression with 0.5 mM IPTG for 3 hr at 28° C. in the presence of 1 mM sodium salicylate (SA; Sigma). Proteins were extracted by mechanical disruption with lysis buffer containing 125 mM Tris HCl (pH 7.5), 150 mM NaCl, cocktail of protease inhibitors, 1 mM PMSF (phenylmethylsulfonyl fluoride, Sigma), 7.15 mM BME (β-mercaptoethanol), 1 mM EDTA, 10 mM NEM (N-ethylmaleimide, Sigma). Expression of proteins was confirmed with SDS-PAGE on the total lysate. To detect ubiquitination of FLAG-WRKY70 under denaturing conditions, 1% SDS was added to the lysate and heated at 95° C. for 10 min, then diluted 10 times with the lysis buffer and subjected to immunoprecipitation (IP) using α-FLAG magnetic agarose beads 888 (ThermoFisher). Eluted proteins were subjected to SDS-PAGE and probed with α-FLAG and α-HA. Table 6 lists all primer sequences used in this study.

Plant transformation: For plant transformations, the Agrobacterium tumefaciens strain GV3101 was transformed with plant binary vectors carrying the indicated transgenes. For stable expression in Arabidopsis, a floral dipping method was used. For transient expression in N benthamiana, the Agrobacterium carrying the indicated construct was cultured overnight at 28° C. in Luria-Bertani (LB) broth medium supplemented with appropriate antibiotics: spectinomycin (100 μg/ml), kanamycin (50 μg/ml), gentamycin (50 μg/ml), and rifampicin (25 μg/ml). The obtained culture was re-inoculated at 1:10 into fresh growth media with antibiotics and grown for another 4 hr. Cells were then spun down at 1,600 g for 10 min, and inoculum was prepared by resuspending cells to OD600 nm=1 in double distilled water (DDW) containing 200 μM acetosyringone (Sigma). Unless otherwise stated, for co-IP, co-localization, and BiFC assays in N. benthamiana, the proteins were co-expressed at 1:1 mixture of the relevant Agrobacteria inoculums. In all BiFC assays, the pair of YN/YC fusions was co-expressed together with free mCherry to mark the cytoplasm and nucleus, mCherry-NLS to mark the nucleus only, or mCherry-fused test proteins for co-localization analysis. The inoculum was pressure infiltrated into N. benthamiana leaves at the abaxial side using 1 ml syringe without the needle. Due to low overall levels of NPR1-GFP in transgenic plants, a transient expression assay in Arabidopsis seedlings was used to monitor NPR1-GFP subcellular localization after SA treatment (FIG. 2B). To limit the imaging time in this transient assay (2 hours), a higher baseline SA concentration had to be used. Seeds of WT (Col-0) Arabidopsis were germinated on solid half-strength Murashige and Skoog (1/2 MS) medium and grown for 4 days. Agrobacterium carrying the indicated constructs was grown overnight in LB medium supplemented with appropriate antibiotics. The obtained culture was re-inoculated at 1:10 into virulence induction medium [50.78 mM MES, 0.5% Glucose, 1.734 mM NaH2PO4, 5% of 20X-AB mix (373.9 mM NH4C1, 24.34 mM MgSO4, 40.23 mM KCl, 1.36 mM CaCl2 and 0.18 mM FeSO4·7H2O), 200 μM acetosyringone] supplemented with 25 μg/ml rifampicin and grown for another 24 hr at 28° C. to OD600=0.8 (inoculum). Seedlings were vacuum-infiltrated with the inoculum, and at 48 hpi (hours post inoculation), treatments were performed by submerging the transformed seedlings in water or SA solution at indicated concentrations for 2 hr. Entire cotyledons were sampled for microscopy.

Chemical treatment, pathogen infection and cell death assays. For soil-grown Arabidopsis plants, SA treatment was carried out with spray at indicated concentrations. For seedlings grown vertically on solid 1/2 MS medium for 10-12 days, SA 925 treatment (1 mM), with or without the proteasome inhibitor MG132 (50 μM), and protein synthesis 926 inhibitor cycloheximide (100 μM) was done by submerging them in solutions for indicated periods. Unless otherwise stated, in N. benthamiana, SA treatment was done by infiltrating leaves 24 hr after agroinfiltration. SA pre-treatment of mature Arabidopsis was done by spray 24 hr before bacterial infection or cell death induction. For bacterial infections, Pseudomonas syringae pv. maculicola ES4326 carrying AvrRpt2 or AvrRpm1 effectors, and Pf Pf0-1 carrying functional AvrRps4 or non-functional AvrRps4KRVY-AAAA effectors were grown for 2 days on solid King's B medium supplemented with appropriate antibiotics. An inoculum was prepared by resuspending cells in 10 mM MgSO4 to obtain the desired optical density: Psm ES4326/AvrRpt2 (OD600 nm=0.02); Psm ES4326/AvrRpm1 (OD600 nm=0.1); Pf Pf0-1/AvrRps4 and Pf Pf0-1/AvrRps4KRVY-AAAA (OD600 nm=0.2). Bacteria were pressure infiltrated into mature leaves of three-week-old Arabidopsis plants and cell death or bacterial growth were assessed at indicated times post inoculation (hpi, hours post inoculation; dpi, days post inoculation). Induction of cell death in dex:AvrRpt2 transgenic plants was performed by spraying plants or infiltrating individual leaves with 25 μM dexamethasone (Sigma). Induction of cell death in est:AvrRpt2 transgenic plants was performed by infiltrating individual leaves with 941 50 μM β-estradiol (Sigma). Heat stress was applied by incubating mature leaf disks from three-942 week-old Arabidopsis plants in 45° C. water bath for 45 min. Oxidative stress was induced by spraying three-week-old Arabidopsis plants with 0.25 mM water solution of MV (Methyl viologen dichloride hydrate; Sigma). UV-C irradiation was performed on leaf disks from three-week-old Arabidopsis plants using UV crosslinker with total dose of 20 kJ/m2. Cell death was monitored using electrolyte leakage assay 1 hr after pathogen infection, or induction of dex:AvrRpt2 and est:AvrRpt2, or application of stresses. For electrolyte leakage, 12 leaf disks were sampled from four plants for each treatment/genotype in three replicates. After sampling, the disks were washed with DDW and conductivity was measured every 3 hr using Orion Star™ A222 Portable Conductivity Meter (ThermoFisher). Monitoring the RPP1/ATR1-induced cell death in the presence of SA was done by addition of SA into the leaf disks incubation solution with subsequent subtraction of the conductivity reads contributed by SA from the total reads. For bacterial growth assay, leaf disks were sampled at 1 dpi from eight infected plants per treatment/genotype, ground in 10 mM MgSO4, and plated with sequential dilutions on King's B medium plates supplemented with appropriate antibiotics. At 2 days, the bacterial colonies were scored.

Trypan blue staining. Leaves of three-week-old Arabidopsis infected with Psm ES4326/AvrRpt2 were sampled at 1 dpi, boiled in 95% ethanol until completely bleached, followed by boiling for 3 min in a 1:1 mixture of 95% ethanol and staining solution (10 ml lactic acid, 10 ml glycerol, 10 g phenol and 10 mg trypan blue, dissolved in 10 ml DDW). The leaves were then de-stained in 2.5 g/ml chloral hydrate solution and transferred to 70% glycerol for subsequent imaging.

VIGS assay. The silencing of NbNPR1 (NbNPR1-RNAi) and NbCUL3 (NbCUL3-RNAi) was done using VIGS assay performed as previously described. Ten-day-old WT N. benthamiana plants were inoculated with Agrobacteria (GV3101) carrying the helper plasmid pTRV1-LIC mixed at 1:1 with a strain carrying either pTRV2-LIC (empty vector control, E.V.), pTRV2-NbPDS (positive control), pTRV2-NbNPR1 or pTRV2-NbCUL3 vectors. Ten days later, gene silencing in the mock and SA-treated plants was verified in the systemic leaves by qPCR using gene-specific and reference gene (eIF4a) primers (Table 6). NbNPR1 silencing was additionally confirmed by analysis of NbPR1 expression. The silenced leaves were used in the standard transient expression assay by agroinfiltration using strains carrying the indicated test genes; and gene expression was assayed at 2 dpi.

Quantitative PCR. Total RNA was extracted from fresh leaf tissue with Trizol reagent (Sigma). cDNA was synthetized using the SuperScript III cDNA Synthesis (Thermofisher). QPCR was performed with FastStart Universal SYBR Green Master Kit (Roche) using Mastercycler ep realplex (Eppendorf). Gene specific primers used for qPCR are listed in Table 6.

PR1 promoter activity and dual luciferase assay. For promoter transactivation assays, the PR1 promoter reporter (pPR1:DUAL-LUC) was transiently co-expressed in N. benthamiana together with free HA, or HA-fused WT NPR1 or npr1 mutants (effectors) followed by treatment with SA at 1 dpi. At 2 dpi (24 hr after SA treatment), leaf discs were collected, ground in liquid nitrogen, and lysed with the PLB buffer of the Dual-Luciferase Reporter Assay System (Promega, E1910). Lysate was spun down at 12,000 g for 1 min, and 10 μl was taken for measuring FLUC and RLUC activities according to manufacturer's instructions using a Victor3 plate reader (PerkinElmer). At 25° C., substrates for FLUC and RLUC were added using the automatic injector and after 3 s shaking and 3 s delay, the signals were captured for 3 s and recorded as counts per second. To obtain the PR1 promoter activity, the ratio of F-LUC and R-LUC activities was calculated for each effector and plotted relative to that of free HA.

Confocal laser scanning microscopy. Unless otherwise indicated, all imaging data and micrographs for single protein imaging, co-localizations, BiFC and time-lapse were obtained from the transient expression in N. benthamiana using the relevant Agrobacteria strains. All imaging was done with confocal laser scanning fluorescence microscopy using Zeiss 880 airyscan inverted confocal microscope with a 40×/1.2 water correction objective. GFP (eGFP) was excited with a 488 nm argon laser and emission was collected with a 505-530 nm band pass filter. YFP was excited with an argon laser using a 514 nm beam splitter, and emission was detected with a 520-555 nm band pass filter. mCherry was excited with a 561 nm diode laser, and emission was detected with a 575-615 nm band pass filter. When eGFP/YFP were imaged together with mCherry, a spectral GASP detector was used to collect emission from eGFP/YFP. Propidium iodide (PI) was excited with 488 nm argon laser and emission was detected with a 590-620 nm band pass filter. Time-lapse imaging was carried out on live leaf tissue samples from N. benthamiana plants transiently expressing the protein of interest. Image acquisition was done in 5 min intervals for the duration of 2 hr by scanning 30 consecutive focal planes along the Z-axis covering the entire thickness of an epidermal cell. Z-projection was done to each time frame stack before composing a final time-lapse file.

Protein analysis and IP. Recombinant protein analysis was performed using transient expression in N. benthamiana, or stable over-expression in transgenic Arabidopsis, or expression in E. coli. Total protein extraction from plants was performed by homogenizing leaf tissue in the plant extraction buffer [50 mM Tris-HCl (pH 7.5), 150 mM NaCl, 5 mM EDTA, 0.2% Triton X-100, 0.2% Nonidet P-40] supplemented with 1 mM PMSF, 100 μM MG132, and EDTA-free protease inhibitor cocktail (Roche). For ubiquitinated protein analysis in plants, 100 μM of DUB (deubiquitinase) inhibitor (LifeSensors) and 10 mM NEM were added. Next, the lysates were cleared by centrifugation at 14,000 rpm for 15 min followed by filtering through a 0.2 μm filter. Unless otherwise indicated, total protein extraction from E. coli was performed by mechanical disruption of cells with lysis buffer containing 125 mM Tris HCl (pH 7.5), 150 mM NaCl, cocktail of protease inhibitors, 1 mM PMSF, 7.15 mM BME, 1 mM EDTA. Pull-downs or IP experiments were done on total protein extracts by incubating protein samples in the lysis buffer overnight with indicated affinity tag or antibody conjugated to agarose or magnetic beads: GFP-trap agarose beads (Chromotek) for GFP fusions; RFP-trap agarose beads (Chromotek) for mCherry fusions; anti-HA magnetic beads (ThermoFisher) for HA tag fusions; anti-DYKDDDDK coupled magnetic agarose (ThermoFisher) for FLAG tag fusions; and glutathione magnetic agarose (ThermoFisher) for GST fusions. Unless otherwise indicated, after pull-down/IP, beads were washed 3 times and proteins were eluted by boiling in the 2×SDS sample buffer. For Western blotting, the SDS sample buffer was added to the protein extracts from a 4×stock solution supplemented with 50 mM DTT (dithiothreitol) and 715 mM BME. Protein samples were heated to 95° C. for 10 min, separated on SDS-PAGE gels, and transferred to nitrocellulose membranes. Western blots were probed with α-AtNPR1, α-AtEDS1 (Agrisera), α-AtNIMIN1 (ABclonal), α-HA (Biolegend), α-Myc (Santa 1036 Cruz), α-FLAG-HRP (Biolegend), α-V5 (Santa Cruz), α-GST HRP (GE Healthcare), α-GFP (Clonetech), α-RFP (Chromotek), α-AtCUL3A, α-Ubiquitin (Ub; P4D1; Santa Cruz), α-TUB (β-Tubulin F1; Santa Cruz), α-Histone H3 (H3; Agrisera), or α-Actin (ACT; Agrisera) primary antibodies. In Western blots, asterisks indicate non-specific bands from the α-NPR1 (FIGS. 1K, 2A, 6D and 6E) and α-HA (FIGS. 4H, 5C and 12C) immunoblots. Equal loading was confirmed by probing either with α-TUB antibody, or by the ˜55 kDa Rubisco band from the Coomassie blue (CB) staining.

Cell fractionation. Cytoplasmic and nuclear proteins were sequentially isolated from three-week-old Arabidopsis (Col-0) plants using 1 g of leaf tissue and a sucrose gradient protocol described previously with modifications. Tissue was homogenized in lysis buffer (20 mM Tris-HCl, pH 7.5, 20 mM KCl, 2 mM EDTA, 2.5 mM MgCl2, 25% glycerol, 0.25 M Sucrose) supplemented with 100 μM MG132, EDTA-free protease inhibitor cocktail, 1 mM PMSF and 5 mM DTT. The lysate was filtered through a 70 μm filter and centrifuged at 20,000 g for 15 min at 4° C., and the supernatant was collected (cytoplasmic fraction). The pellet was washed four times with 5 ml of NRBT buffer (20 mM Tris-HCl, pH 7.4, 25% glycerol, 2.5 mM MgCl2, and 0.2% Triton X-100). After the last wash, the pellet was resuspended with 500 μl of NRB2 buffer (20 mM Tris-HCl, pH 7.5, 0.25 M Sucrose, 10 mM MgCl2, 0.5% Triton X-100) supplemented with protease inhibitor cocktail and 5 mM BME. The obtained suspension was layered at 1:1 on top of the NRB3 buffer (20 mM Tris-HCl, pH 7.5, 1.7 M Sucrose, 10 mM MgCl2, 0.5% Triton X-100) supplemented with protease inhibitor cocktail and 5 mM BME, centrifuged at 16,000 g for 45 min at 4° C. The top layer was removed and the pellet was resuspended with 200 μl of plant extraction buffer containing 1% Triton X-100, protease inhibitor cocktail and 5 mM BME (nuclear fraction). Samples were run on a reducing SDS-PAGE. Cell fractionation was confirmed by immunoblotting with antibodies against cytoplasmic marker actin (α-ACT) and nuclear marker histone H3 (α-H3).

Prediction of redox-sensitive disorder regions. Protein sequences of AtNPRs were submitted to the IUPred2a tool (iupred2a.elte.hu/; (Meszaros et al., 2018)) for prediction of intrinsically disordered regions (IDRs) with experimental redox-state option turned on. The differential IDR score per residue (FIG. 2F) was calculated by subtracting the predicted redox-plus (WT protein) values from redox-minus (mutated cysteines) values (Table 3).

Mass spectrometry. For proteomic analysis of NPR1 condensates, total proteins from mock- and SA-treated transgenic Arabidopsis plants expressing sim3-GFP in the WT NPR1 background (sim3-GFP/Col-0) were isolated, followed by immunoprecipitation of sim3-GFP and a quantitative LC-MS analysis of the co-purified proteins. Lysates were prepared from 6 g of tissue from three-week-old plants treated with water (mock) or 1 mM SA for 24 hr using IP buffer (plant extraction buffer containing 1% Triton X-100) supplemented with 1 mM PMSF, 100 μM MG132, 100 μM DUB inhibitor, 10 mM NEM, 1.43 mM BME, EDTA-free protease inhibitor cocktail (Roche), and 100 μM SA for SA-treated sample. For each treatment the obtained lysate was filtered through a 0.2 μm filter, split into three replicates, mixed with GFP-trap agarose beads (Chromotek) under saturating conditions [25 μl beads (50% slurry)/3 ml of lysate] and subjected to three independent IP reactions per each sample by overnight incubation at 4° C. After incubation, the beads were washed five times with the IP buffer and three times with 50 mM ammonium bicarbonate (NH4HCO3). For silver stain, 5% of the beads were mixed with ×2 SDS sample buffer, boiled at 95° C. for 10 min and the supernatant was run on a 4-12% polyacrylamide gel. The gel was stained with Pierce™ Silver Stain Kit (ThermoFisher) according to the manufacturer's instructions. The remaining beads were submitted to the Duke Proteomics Core Facility for an on-bead trypsin digestion, peptide lyophilization and LC-MS/MS analysis. Quantitative one-dimensional liquid chromatography, tandem mass spectrometry (1D-LC-MS/MS) was performed on equal volumes of peptide digests of the three replicates from each treatment. Following the LC-MS/MS runs, data were imported into Rosetta Elucidator v. 4 (Rosetta Biosoftware, Inc.), and analyses were aligned based on the accurate mass and retention time of detected ions (“features”) using PeakTeller algorithm in Elucidator. Relative peptide abundance (expression intensity) was calculated based on area-under-the curve (AUC) of the selected ion chromatograms of the aligned features across all runs. The MS/MS data were searched against a custom Araport11 database with an additional entry for the sim3-GFP sequence and an equal number of reversed-sequence “decoys” for false discovery rate determination (96,720 total entries). Mascot Distiller and Mascot Server (v 2.5, Matrix Sciences) were utilized to produce fragment ion spectra and to perform the database searches. Database search parameters included precursor mass tolerance of 5 ppm, product ion mass tolerance of 0.8 Da, trypsin specificity with up to 2 missed cleavages, fixed modification on Cys (carbamidomethyl) and variable modification of deamidation (Asn/Gln), oxidation (Met) and N-terminal protein acetylation. After individual peptide scoring using the PeptideProphet algorithm in Elucidator, the data were annotated at a 1% peptide and 0.8% protein false discovery rates, respectively. The data were filtered to remove low quality peptides with poor chromatographic peak shape, and those quantified by less than two peptides. Only those proteins quantified from at least two replicates in each sample were accepted. Due to differences in the intensity values of sim3-GFP peptides between mock and SA treated samples, the data were normalized to the mean sim3-GFP expression across all 6 samples. In the normalized data the intensities of sim3-GFP were equalized, while other identified protein intensities varied based on their abundance. The variability between the samples and replicates were visualized from the normalized data by principal component analysis (PCA) and 2-dimentional hierarchical clustering (FIGS. 11A-11H). Fold change (FC) of the mean intensities from the three replicates of SA-treated samples over mock was calculated and a t-test was applied to calculate the p-value. The final list of sim3-GFP interactors (171 proteins) was obtained by applying FC cut-off above 2, and a p-value below 0.05. The GO term analysis of the interactome list was performed using the ShinyGO v0.60: Gene Ontology Enrichment Analysis tool (Ge et al., 2019). For intersection analysis of SINC functional groups, the UpSet plot (FIG. 3A) was generated using Intervene Shiny App.

Quantification And Statistical Analysis. For all image quantifications, 8-16 randomly sampled unsaturated confocal images (512×512 pixels, 225×225 μm) were used with an automated image analysis algorithm implemented in the ImageJ software as previously described. Briefly, for each experiment, images from control and test samples were grouped into a dataset. Next, a random image was selected from the dataset and parameters such as local threshold, background noise, object size and shape were determined. The obtained parameters were fed into an algorithm for automated analysis of the entire dataset. The output values, such as body number, size, and intensity/body, were used for statistical analysis between test and control groups using Student's t tests or one-way ANOVA tests. For quantification of co-localization, Pearson's correlation coefficients were calculated between GFP/BiFC and mCherry signals using the JACoP plugin in the ImageJ software with the default settings.

All experiments involving measurements/quantifications, imaging and quantifications from images were repeated at least two times with similar results. Data plotting and statistical tests were performed in GraphPad Prism 8. Statistical parameters such as mean±SD (standard deviation), SE (standard error), 95% confidence intervals are indicated in figure legends. In graphs showing quantification of co-localization and nuclear-cytoplasmic partitioning, all data points (number of images used, n) are plotted using box & whiskers with min/max range indication. In all graphs, asterisks indicate statistical significance (*, p<0.05; **, p<0.01; ***, p<0.001; ****, p<0.0001, ns, not significant) tested by Student's t test (two groups) or one/two-way ANOVA (multiple groups). Lowercase letters indicate statistical significance tested between multiple groups by one-way ANOVA at p<0.05. In conductivity assays n=3; in bacterial growth assays n=8; in PR1 promoter transactivation assay n=3.

### TABLES

TABLE 3
Prediction Scores for Redox-Sensitive Disorder Regions (RDRs) in Arabidopsis NPRs.
Related to FIGS. 2A-2I and 9A-9E. The RDR regions were predicted with the IUPred2a algorithm
(iupred2a.elte.hu/). Differential IDR score was calculated by subtracting the redox-plus
(WT protein) scores from the redox-minus (mutated cysteines) scores for each residue.

| | | IUPRED2 | IUPRED2 | REDOX SENSITIVE | DIFFERENTIAL IDR |
|---|---|---|---|---|---|
| AMINO ACID | AMINO | REDOX | REDOX | DISORDER | SCORE [(REDOX MINUS) − |
| POSITION | ACID | PLUX | MINUS | REGION, RDR (1) | (REDOX PLUS)] |
| >AtNPR1 | | | | | |
| (AT1G64280) | | | | | |
| 1 | M | 0.8595 | 0.8595 | 0 | 0 |
| 2 | D | 0.7573 | 0.7573 | 0 | 0 |
| 3 | T | 0.7458 | 0.7458 | 0 | 0 |
| 4 | T | 0.6412 | 0.6412 | 0 | 0 |
| 5 | I | 0.5941 | 0.5941 | 0 | 0 |
| 6 | D | 0.5514 | 0.5514 | 0 | 0 |
| 7 | G | 0.5008 | 0.5008 | 0 | 0 |
| 8 | F | 0.4556 | 0.4556 | 0 | 0 |
| 9 | A | 0.3359 | 0.3359 | 0 | 0 |
| 10 | D | 0.2385 | 0.2385 | 0 | 0 |
| 11 | S | 0.1698 | 0.1698 | 0 | 0 |
| 12 | Y | 0.1844 | 0.1844 | 0 | 0 |
| 13 | E | 0.1921 | 0.1921 | 0 | 0 |
| 14 | I | 0.1998 | 0.1998 | 0 | 0 |
| 15 | S | 0.2041 | 0.2041 | 0 | 0 |
| 16 | S | 0.282 | 0.282 | 0 | 0 |
| 17 | T | 0.27 | 0.27 | 0 | 0 |
| 18 | S | 0.2602 | 0.2602 | 0 | 0 |
| 19 | F | 0.27 | 0.27 | 0 | 0 |
| 20 | V | 0.2483 | 0.2483 | 0 | 0 |
| 21 | A | 0.1698 | 0.1698 | 0 | 0 |
| 22 | T | 0.1178 | 0.1178 | 0 | 0 |
| 23 | D | 0.1456 | 0.1456 | 0 | 0 |
| 24 | N | 0.1018 | 0.1018 | 0 | 0 |
| 25 | T | 0.1766 | 0.1766 | 0 | 0 |
| 26 | D | 0.1921 | 0.1921 | 0 | 0 |
| 27 | S | 0.1456 | 0.1456 | 0 | 0 |
| 28 | S | 0.1018 | 0.1018 | 0 | 0 |
| 29 | I | 0.0991 | 0.0991 | 0 | 0 |
| 30 | V | 0.1532 | 0.1532 | 0 | 0 |
| 31 | Y | 0.2292 | 0.2292 | 0 | 0 |
| 32 | L | 0.2786 | 0.2786 | 0 | 0 |
| 33 | A | 0.2209 | 0.2209 | 0 | 0 |
| 34 | A | 0.1998 | 0.1998 | 0 | 0 |
| 35 | E | 0.1698 | 0.1698 | 0 | 0 |
| 36 | Q | 0.1178 | 0.1178 | 0 | 0 |
| 37 | V | 0.1178 | 0.1178 | 0 | 0 |
| 38 | L | 0.0744 | 0.0744 | 0 | 0 |
| 39 | T | 0.0478 | 0.0478 | 0 | 0 |
| 40 | G | 0.0789 | 0.0789 | 0 | 0 |
| 41 | P | 0.115 | 0.115 | 0 | 0 |
| 42 | D | 0.1635 | 0.1635 | 0 | 0 |
| 43 | V | 0.1456 | 0.1456 | 0 | 0 |
| 44 | S | 0.1921 | 0.1921 | 0 | 0 |
| 45 | A | 0.2209 | 0.2209 | 0 | 0 |
| 46 | L | 0.1456 | 0.1456 | 0 | 0 |
| 47 | Q | 0.0789 | 0.0789 | 0 | 0 |
| 48 | L | 0.124 | 0.124 | 0 | 0 |
| 49 | L | 0.1766 | 0.1766 | 0 | 0 |
| 50 | S | 0.2122 | 0.2167 | 0 | 0.0045 |
| 51 | N | 0.2209 | 0.2255 | 0 | 0.0046 |
| 52 | S | 0.1998 | 0.2041 | 0 | 0.0043 |
| 53 | F | 0.1117 | 0.1178 | 0 | 0.0061 |
| 54 | E | 0.115 | 0.1178 | 0 | 0.0028 |
| 55 | S | 0.115 | 0.1178 | 0 | 0.0028 |
| 56 | V | 0.1456 | 0.1532 | 0 | 0.0076 |
| 57 | F | 0.1805 | 0.1878 | 0 | 0.0073 |
| 58 | D | 0.1635 | 0.1698 | 0 | 0.0063 |
| 59 | S | 0.1602 | 0.1698 | 0 | 0.0096 |
| 60 | P | 0.1667 | 0.1766 | 0 | 0.0099 |
| 61 | D | 0.1088 | 0.1178 | 0 | 0.009 |
| 62 | D | 0.1018 | 0.1088 | 0 | 0.007 |
| 63 | F | 0.106 | 0.1178 | 0 | 0.0118 |
| 64 | Y | 0.1805 | 0.1958 | 0 | 0.0153 |
| 65 | S | 0.1602 | 0.1766 | 0 | 0.0164 |
| 66 | D | 0.1732 | 0.1921 | 0 | 0.0189 |
| 67 | A | 0.1732 | 0.1921 | 0 | 0.0189 |
| 68 | K | 0.2558 | 0.2748 | 0 | 0.019 |
| 69 | L | 0.1566 | 0.1732 | 0 | 0.0166 |
| 70 | V | 0.1602 | 0.1805 | 0 | 0.0203 |
| 71 | L | 0.1322 | 0.1495 | 0 | 0.0173 |
| 72 | S | 0.0858 | 0.1349 | 0 | 0.0491 |
| 73 | D | 0.0567 | 0.0965 | 0 | 0.0398 |
| 74 | G | 0.0723 | 0.1178 | 0 | 0.0455 |
| 75 | R | 0.1041 | 0.1566 | 0 | 0.0525 |
| 76 | E | 0.0884 | 0.138 | 0 | 0.0496 |
| 77 | V | 0.0832 | 0.1322 | 0 | 0.049 |
| 78 | S | 0.1018 | 0.1532 | 0 | 0.0514 |
| 79 | F | 0.1041 | 0.1566 | 0 | 0.0525 |
| 80 | H | 0.0965 | 0.1456 | 0 | 0.0491 |
| 81 | R | 0.0832 | 0.1292 | 0 | 0.046 |
| 82 | S | 0.1349 | 0.1958 | 0 | 0.0609 |
| 83 | V | 0.138 | 0.1998 | 0 | 0.0618 |
| 84 | L | 0.115 | 0.1698 | 0 | 0.0548 |
| 85 | S | 0.0771 | 0.1205 | 0 | 0.0434 |
| 86 | A | 0.0643 | 0.1041 | 0 | 0.0398 |
| 87 | R | 0.0502 | 0.0813 | 0 | 0.0311 |
| 88 | S | 0.0621 | 0.1041 | 0 | 0.042 |
| 89 | S | 0.0643 | 0.106 | 0 | 0.0417 |
| 90 | F | 0.1088 | 0.1667 | 0 | 0.0579 |
| 91 | F | 0.1178 | 0.1732 | 0 | 0.0554 |
| 92 | K | 0.1178 | 0.1732 | 0 | 0.0554 |
| 93 | S | 0.1667 | 0.1844 | 0 | 0.0177 |
| 94 | A | 0.2041 | 0.2209 | 0 | 0.0168 |
| 95 | L | 0.27 | 0.2865 | 0 | 0.0165 |
| 96 | A | 0.2786 | 0.3005 | 0 | 0.0219 |
| 97 | A | 0.3005 | 0.3225 | 0 | 0.022 |
| 98 | A | 0.2657 | 0.2865 | 0 | 0.0208 |
| 99 | K | 0.2333 | 0.2558 | 0 | 0.0225 |
| 100 | K | 0.1766 | 0.1958 | 0 | 0.0192 |
| 101 | E | 0.2531 | 0.2748 | 0 | 0.0217 |
| 102 | K | 0.2602 | 0.282 | 0 | 0.0218 |
| 103 | D | 0.27 | 0.2913 | 0 | 0.0213 |
| 104 | S | 0.1958 | 0.2122 | 0 | 0.0164 |
| 105 | N | 0.2167 | 0.2333 | 0 | 0.0166 |
| 106 | N | 0.2913 | 0.3053 | 0 | 0.014 |
| 107 | T | 0.2333 | 0.2483 | 0 | 0.015 |
| 108 | A | 0.2292 | 0.2385 | 0 | 0.0093 |
| 109 | A | 0.2531 | 0.2602 | 0 | 0.0071 |
| 110 | V | 0.27 | 0.2748 | 0 | 0.0048 |
| 111 | K | 0.2041 | 0.2122 | 0 | 0.0081 |
| 112 | L | 0.1958 | 0.2041 | 0 | 0.0083 |
| 113 | E | 0.1532 | 0.1566 | 0 | 0.0034 |
| 114 | L | 0.1532 | 0.1566 | 0 | 0.0034 |
| 115 | K | 0.0991 | 0.1018 | 0 | 0.0027 |
| 116 | E | 0.1041 | 0.106 | 0 | 0.0019 |
| 117 | I | 0.0991 | 0.1018 | 0 | 0.0027 |
| 118 | A | 0.0744 | 0.0771 | 0 | 0.0027 |
| 119 | K | 0.066 | 0.0677 | 0 | 0.0017 |
| 120 | D | 0.0813 | 0.0832 | 0 | 0.0019 |
| 121 | Y | 0.0858 | 0.0935 | 0 | 0.0077 |
| 122 | E | 0.0607 | 0.0677 | 0 | 0.007 |
| 123 | V | 0.0832 | 0.0965 | 0 | 0.0133 |
| 124 | G | 0.0502 | 0.0587 | 0 | 0.0085 |
| 125 | F | 0.0587 | 0.0723 | 0 | 0.0136 |
| 126 | D | 0.0405 | 0.0514 | 0 | 0.0109 |
| 127 | S | 0.0395 | 0.049 | 0 | 0.0095 |
| 128 | V | 0.066 | 0.0813 | 0 | 0.0153 |
| 129 | V | 0.0789 | 0.0991 | 0 | 0.0202 |
| 130 | T | 0.0567 | 0.0744 | 0 | 0.0177 |
| 131 | V | 0.0502 | 0.066 | 0 | 0.0158 |
| 132 | L | 0.0884 | 0.1178 | 0 | 0.0294 |
| 133 | A | 0.0965 | 0.1292 | 0 | 0.0327 |
| 134 | Y | 0.1456 | 0.1878 | 0 | 0.0422 |
| 135 | V | 0.1349 | 0.1805 | 0 | 0.0456 |
| 136 | Y | 0.1998 | 0.2657 | 0 | 0.0659 |
| 137 | S | 0.1349 | 0.1921 | 0 | 0.0572 |
| 138 | S | 0.1349 | 0.1878 | 0 | 0.0529 |
| 139 | R | 0.1878 | 0.2531 | 0 | 0.0653 |
| 140 | V | 0.1266 | 0.3005 | 1 | 0.1739 |
| 141 | R | 0.1018 | 0.2657 | 1 | 0.1639 |
| 142 | P | 0.1456 | 0.3359 | 1 | 0.1903 |
| 143 | P | 0.208 | 0.4037 | 1 | 0.1957 |
| 144 | P | 0.2385 | 0.4379 | 1 | 0.1994 |
| 145 | K | 0.1998 | 0.4967 | 1 | 0.2969 |
| 146 | G | 0.1602 | 0.5412 | 1 | 0.381 |
| 147 | V | 0.1998 | 0.6035 | 1 | 0.4037 |
| 148 | S | 0.1349 | 0.5296 | 1 | 0.3947 |
| 149 | E | 0.1088 | 0.5008 | 1 | 0.392 |
| 150 | S | 0.0514 | 0.5008 | 1 | 0.4494 |
| 151 | A | 0.0677 | 0.5412 | 1 | 0.4735 |
| 152 | D | 0.0832 | 0.5711 | 1 | 0.4879 |
| 153 | E | 0.0567 | 0.5253 | 1 | 0.4686 |
| 154 | N | 0.0316 | 0.4282 | 1 | 0.3966 |
| 155 | S | 0.0308 | 0.4245 | 1 | 0.3937 |
| 156 | S | 0.0157 | 0.3359 | 1 | 0.3202 |
| 157 | H | 0.0121 | 0.2963 | 1 | 0.2842 |
| 158 | V | 0.009 | 0.2657 | 1 | 0.2567 |
| 159 | A | 0.0099 | 0.282 | 1 | 0.2721 |
| 160 | S | 0.0061 | 0.1958 | 0 | 0.1897 |
| 161 | R | 0.0074 | 0.1266 | 0 | 0.1192 |
| 162 | P | 0.0055 | 0.0991 | 0 | 0.0936 |
| 163 | A | 0.0015 | 0.0478 | 0 | 0.0463 |
| 164 | V | 0.0009 | 0.035 | 0 | 0.0341 |
| 165 | D | 0.0005 | 0.0144 | 0 | 0.0139 |
| 166 | F | 0.0006 | 0.0078 | 0 | 0.0072 |
| 167 | M | 0.0005 | 0.0033 | 0 | 0.0028 |
| 168 | L | 0.0005 | 0.0029 | 0 | 0.0024 |
| 169 | E | 0.0004 | 0.0021 | 0 | 0.0017 |
| 170 | V | 0.0006 | 0.0035 | 0 | 0.0029 |
| 171 | L | 0.0013 | 0.0042 | 0 | 0.0029 |
| 172 | Y | 0.0007 | 0.0012 | 0 | 0.0005 |
| 173 | L | 0.0003 | 0.0005 | 0 | 0.0002 |
| 174 | A | 0.0004 | 0.0006 | 0 | 0.0002 |
| 175 | F | 0.0003 | 0.0005 | 0 | 0.0002 |
| 176 | I | 0.0002 | 0.0003 | 0 | 0.0001 |
| 177 | F | 0.0003 | 0.0004 | 0 | 0.0001 |
| 178 | K | 0.0003 | 0.0005 | 0 | 0.0002 |
| 179 | I | 0.0005 | 0.0007 | 0 | 0.0002 |
| 180 | P | 0.0002 | 0.0004 | 0 | 0.0002 |
| 181 | E | 0.0002 | 0.0003 | 0 | 0.0001 |
| 182 | L | 0.0003 | 0.0005 | 0 | 0.0002 |
| 183 | I | 0.0003 | 0.0004 | 0 | 0.0001 |
| 184 | T | 0.0003 | 0.0004 | 0 | 0.0001 |
| 185 | L | 0.0004 | 0.0005 | 0 | 0.0001 |
| 186 | Y | 0.0007 | 0.0009 | 0 | 0.0002 |
| 187 | Q | 0.0008 | 0.0009 | 0 | 1E−04 |
| 188 | R | 0.0009 | 0.0013 | 0 | 0.0004 |
| 189 | H | 0.0006 | 0.0007 | 0 | 0.0001 |
| 190 | L | 0.001 | 0.0015 | 0 | 0.0005 |
| 191 | L | 0.0008 | 0.001 | 0 | 0.0002 |
| 192 | D | 0.0008 | 0.0009 | 0 | 1E−04 |
| 193 | V | 0.0008 | 0.001 | 0 | 0.0002 |
| 194 | V | 0.001 | 0.0024 | 0 | 0.0014 |
| 195 | D | 0.0007 | 0.0008 | 0 | 0.0001 |
| 196 | K | 0.0007 | 0.0009 | 0 | 0.0002 |
| 197 | V | 0.0013 | 0.0028 | 0 | 0.0015 |
| 198 | V | 0.0007 | 0.0009 | 0 | 0.0002 |
| 199 | I | 0.0006 | 0.0008 | 0 | 0.0002 |
| 200 | E | 0.0007 | 0.0009 | 0 | 0.0002 |
| 201 | D | 0.0007 | 0.0009 | 0 | 0.0002 |
| 202 | T | 0.0008 | 0.0037 | 0 | 0.0029 |
| 203 | L | 0.0008 | 0.0035 | 0 | 0.0027 |
| 204 | V | 0.0018 | 0.0075 | 0 | 0.0057 |
| 205 | I | 0.0031 | 0.0099 | 0 | 0.0068 |
| 206 | L | 0.001 | 0.0097 | 0 | 0.0087 |
| 207 | K | 0.0009 | 0.0084 | 0 | 0.0075 |
| 208 | L | 0.0024 | 0.0157 | 0 | 0.0133 |
| 209 | A | 0.0024 | 0.016 | 0 | 0.0136 |
| 210 | N | 0.0028 | 0.02 | 0 | 0.0172 |
| 211 | I | 0.0028 | 0.0212 | 0 | 0.0184 |
| 212 | S | 0.0028 | 0.02 | 0 | 0.0172 |
| 213 | G | 0.001 | 0.0212 | 0 | 0.0202 |
| 214 | K | 0.0033 | 0.0414 | 0 | 0.0381 |
| 215 | A | 0.0067 | 0.0723 | 0 | 0.0656 |
| 216 | S | 0.0069 | 0.0744 | 0 | 0.0675 |
| 217 | M | 0.0078 | 0.0813 | 0 | 0.0735 |
| 218 | K | 0.0049 | 0.0526 | 0 | 0.0477 |
| 219 | L | 0.0094 | 0.1018 | 0 | 0.0924 |
| 220 | L | 0.0137 | 0.1266 | 0 | 0.1129 |
| 221 | D | 0.0131 | 0.1266 | 0 | 0.1135 |
| 222 | R | 0.0179 | 0.1566 | 0 | 0.1387 |
| 223 | S | 0.0376 | 0.1566 | 0 | 0.119 |
| 224 | K | 0.0336 | 0.1416 | 0 | 0.108 |
| 225 | E | 0.0226 | 0.1041 | 0 | 0.0815 |
| 226 | I | 0.0316 | 0.1322 | 0 | 0.1006 |
| 227 | I | 0.035 | 0.0884 | 0 | 0.0534 |
| 228 | V | 0.0425 | 0.1041 | 0 | 0.0616 |
| 229 | K | 0.0441 | 0.1088 | 0 | 0.0647 |
| 230 | S | 0.0813 | 0.1698 | 0 | 0.0885 |
| 231 | N | 0.0858 | 0.1766 | 0 | 0.0908 |
| 232 | V | 0.0991 | 0.1958 | 0 | 0.0967 |
| 233 | D | 0.0991 | 0.1921 | 0 | 0.093 |
| 234 | M | 0.1602 | 0.1878 | 0 | 0.0276 |
| 235 | V | 0.1117 | 0.1349 | 0 | 0.0232 |
| 236 | S | 0.0771 | 0.0965 | 0 | 0.0194 |
| 237 | L | 0.1322 | 0.1635 | 0 | 0.0313 |
| 238 | E | 0.2041 | 0.2385 | 0 | 0.0344 |
| 239 | K | 0.1958 | 0.2292 | 0 | 0.0334 |
| 240 | S | 0.1349 | 0.1602 | 0 | 0.0253 |
| 241 | L | 0.1349 | 0.1602 | 0 | 0.0253 |
| 242 | P | 0.1349 | 0.1566 | 0 | 0.0217 |
| 243 | E | 0.1805 | 0.2041 | 0 | 0.0236 |
| 244 | E | 0.1766 | 0.1958 | 0 | 0.0192 |
| 245 | L | 0.1958 | 0.2122 | 0 | 0.0164 |
| 246 | V | 0.1878 | 0.2041 | 0 | 0.0163 |
| 247 | K | 0.1921 | 0.208 | 0 | 0.0159 |
| 248 | E | 0.1958 | 0.2041 | 0 | 0.0083 |
| 249 | I | 0.1958 | 0.208 | 0 | 0.0122 |
| 250 | I | 0.1495 | 0.1566 | 0 | 0.0071 |
| 251 | D | 0.1698 | 0.1766 | 0 | 0.0068 |
| 252 | R | 0.2385 | 0.2432 | 0 | 0.0047 |
| 253 | R | 0.1602 | 0.1635 | 0 | 0.0033 |
| 254 | K | 0.1602 | 0.1635 | 0 | 0.0033 |
| 255 | E | 0.1566 | 0.1602 | 0 | 0.0036 |
| 256 | L | 0.2292 | 0.2292 | 0 | 0 |
| 257 | G | 0.2255 | 0.2255 | 0 | 0 |
| 258 | L | 0.2292 | 0.2292 | 0 | 0 |
| 259 | E | 0.2385 | 0.2385 | 0 | 0 |
| 260 | V | 0.2602 | 0.2602 | 0 | 0 |
| 261 | P | 0.3491 | 0.3491 | 0 | 0 |
| 262 | K | 0.3359 | 0.3359 | 0 | 0 |
| 263 | V | 0.3184 | 0.3184 | 0 | 0 |
| 264 | K | 0.2432 | 0.2483 | 0 | 0.0051 |
| 265 | K | 0.2432 | 0.2531 | 0 | 0.0099 |
| 266 | H | 0.2432 | 0.2483 | 0 | 0.0051 |
| 267 | V | 0.3096 | 0.3184 | 0 | 0.0088 |
| 268 | S | 0.3005 | 0.3096 | 0 | 0.0091 |
| 269 | N | 0.282 | 0.2913 | 0 | 0.0093 |
| 270 | V | 0.2913 | 0.3005 | 0 | 0.0092 |
| 271 | H | 0.27 | 0.282 | 0 | 0.012 |
| 272 | K | 0.1805 | 0.1921 | 0 | 0.0116 |
| 273 | A | 0.1805 | 0.1958 | 0 | 0.0153 |
| 274 | L | 0.1667 | 0.1844 | 0 | 0.0177 |
| 275 | D | 0.1178 | 0.1322 | 0 | 0.0144 |
| 276 | S | 0.0771 | 0.0909 | 0 | 0.0138 |
| 277 | D | 0.0723 | 0.0858 | 0 | 0.0135 |
| 278 | D | 0.1088 | 0.1292 | 0 | 0.0204 |
| 279 | I | 0.1041 | 0.124 | 0 | 0.0199 |
| 280 | E | 0.0965 | 0.124 | 0 | 0.0275 |
| 281 | L | 0.1266 | 0.1602 | 0 | 0.0336 |
| 282 | V | 0.1205 | 0.1602 | 0 | 0.0397 |
| 283 | K | 0.0723 | 0.1018 | 0 | 0.0295 |
| 284 | L | 0.0832 | 0.115 | 0 | 0.0318 |
| 285 | L | 0.1322 | 0.1698 | 0 | 0.0376 |
| 286 | L | 0.1117 | 0.1495 | 0 | 0.0378 |
| 287 | K | 0.0587 | 0.1456 | 0 | 0.0869 |
| 288 | E | 0.0502 | 0.124 | 0 | 0.0738 |
| 289 | D | 0.0279 | 0.0723 | 0 | 0.0444 |
| 290 | H | 0.0464 | 0.1266 | 0 | 0.0802 |
| 291 | T | 0.0218 | 0.0621 | 0 | 0.0403 |
| 292 | N | 0.0316 | 0.0935 | 0 | 0.0619 |
| 293 | L | 0.0308 | 0.0909 | 0 | 0.0601 |
| 294 | D | 0.0268 | 0.0771 | 0 | 0.0503 |
| 295 | D | 0.0279 | 0.0789 | 0 | 0.051 |
| 296 | A | 0.0226 | 0.124 | 0 | 0.1014 |
| 297 | S | 0.0387 | 0.1844 | 0 | 0.1457 |
| 298 | A | 0.0259 | 0.1349 | 0 | 0.109 |
| 299 | L | 0.0231 | 0.1266 | 0 | 0.1035 |
| 300 | H | 0.0231 | 0.1266 | 0 | 0.1035 |
| 301 | F | 0.0179 | 0.1041 | 0 | 0.0862 |
| 302 | A | 0.0182 | 0.106 | 0 | 0.0878 |
| 303 | V | 0.0182 | 0.1041 | 0 | 0.0859 |
| 304 | A | 0.02 | 0.1088 | 0 | 0.0888 |
| 305 | Y | 0.0116 | 0.0677 | 0 | 0.0561 |
| 306 | S | 0.0118 | 0.0701 | 0 | 0.0583 |
| 307 | N | 0.0083 | 0.0526 | 0 | 0.0443 |
| 308 | V | 0.0167 | 0.0526 | 0 | 0.0359 |
| 309 | K | 0.0118 | 0.0405 | 0 | 0.0287 |
| 310 | T | 0.0179 | 0.0567 | 0 | 0.0388 |
| 311 | A | 0.02 | 0.0587 | 0 | 0.0387 |
| 312 | T | 0.0259 | 0.0723 | 0 | 0.0464 |
| 313 | D | 0.0316 | 0.0884 | 0 | 0.0568 |
| 314 | L | 0.0464 | 0.1266 | 0 | 0.0802 |
| 315 | L | 0.0554 | 0.1456 | 0 | 0.0902 |
| 316 | K | 0.0909 | 0.208 | 0 | 0.1171 |
| 317 | L | 0.1844 | 0.2385 | 0 | 0.0541 |
| 318 | D | 0.1844 | 0.2333 | 0 | 0.0489 |
| 319 | L | 0.2432 | 0.2963 | 0 | 0.0531 |
| 320 | A | 0.1698 | 0.2209 | 0 | 0.0511 |
| 321 | D | 0.1766 | 0.2255 | 0 | 0.0489 |
| 322 | V | 0.1602 | 0.1998 | 0 | 0.0396 |
| 323 | N | 0.1117 | 0.1416 | 0 | 0.0299 |
| 324 | H | 0.1178 | 0.1495 | 0 | 0.0317 |
| 325 | R | 0.1292 | 0.1602 | 0 | 0.031 |
| 326 | N | 0.1698 | 0.1998 | 0 | 0.03 |
| 327 | P | 0.1495 | 0.1766 | 0 | 0.0271 |
| 328 | R | 0.1921 | 0.2209 | 0 | 0.0288 |
| 329 | G | 0.1878 | 0.2167 | 0 | 0.0289 |
| 330 | Y | 0.27 | 0.2963 | 0 | 0.0263 |
| 331 | T | 0.3184 | 0.3399 | 0 | 0.0215 |
| 332 | V | 0.3491 | 0.3668 | 0 | 0.0177 |
| 333 | L | 0.4282 | 0.4458 | 0 | 0.0176 |
| 334 | H | 0.3535 | 0.3668 | 0 | 0.0133 |
| 335 | V | 0.282 | 0.2913 | 0 | 0.0093 |
| 336 | A | 0.2167 | 0.2255 | 0 | 0.0088 |
| 337 | A | 0.2167 | 0.2209 | 0 | 0.0042 |
| 338 | M | 0.1349 | 0.1416 | 0 | 0.0067 |
| 339 | R | 0.0909 | 0.0965 | 0 | 0.0056 |
| 340 | K | 0.1018 | 0.1018 | 0 | 0 |
| 341 | E | 0.1566 | 0.1566 | 0 | 0 |
| 342 | P | 0.1566 | 0.1566 | 0 | 0 |
| 343 | Q | 0.1732 | 0.1766 | 0 | 0.0034 |
| 344 | L | 0.2483 | 0.2483 | 0 | 0 |
| 345 | I | 0.2041 | 0.2041 | 0 | 0 |
| 346 | L | 0.27 | 0.27 | 0 | 0 |
| 347 | S | 0.3096 | 0.3096 | 0 | 0 |
| 348 | L | 0.3005 | 0.3053 | 0 | 0.0048 |
| 349 | L | 0.3311 | 0.3399 | 0 | 0.0088 |
| 350 | E | 0.2602 | 0.27 | 0 | 0.0098 |
| 351 | K | 0.27 | 0.2786 | 0 | 0.0086 |
| 352 | G | 0.2558 | 0.27 | 0 | 0.0142 |
| 353 | A | 0.2292 | 0.2483 | 0 | 0.0191 |
| 354 | S | 0.2167 | 0.2385 | 0 | 0.0218 |
| 355 | A | 0.2602 | 0.2865 | 0 | 0.0263 |
| 356 | S | 0.27 | 0.3005 | 0 | 0.0305 |
| 357 | E | 0.3225 | 0.3535 | 0 | 0.031 |
| 358 | A | 0.2385 | 0.27 | 0 | 0.0315 |
| 359 | T | 0.27 | 0.3096 | 0 | 0.0396 |
| 360 | L | 0.3359 | 0.3762 | 0 | 0.0403 |
| 361 | E | 0.3225 | 0.3717 | 0 | 0.0492 |
| 362 | G | 0.282 | 0.3399 | 0 | 0.0579 |
| 363 | R | 0.282 | 0.3399 | 0 | 0.0579 |
| 364 | T | 0.2963 | 0.3578 | 0 | 0.0615 |
| 365 | A | 0.2657 | 0.3359 | 0 | 0.0702 |
| 366 | L | 0.2483 | 0.3146 | 0 | 0.0663 |
| 367 | M | 0.2558 | 0.3263 | 0 | 0.0705 |
| 368 | I | 0.1495 | 0.3184 | 1 | 0.1689 |
| 369 | A | 0.1732 | 0.3535 | 1 | 0.1803 |
| 370 | K | 0.1698 | 0.3535 | 1 | 0.1837 |
| 371 | Q | 0.1766 | 0.3578 | 1 | 0.1812 |
| 372 | A | 0.1998 | 0.3806 | 1 | 0.1808 |
| 373 | T | 0.208 | 0.3885 | 1 | 0.1805 |
| 374 | M | 0.2041 | 0.3885 | 1 | 0.1844 |
| 375 | A | 0.124 | 0.3939 | 1 | 0.2699 |
| 376 | V | 0.138 | 0.4149 | 1 | 0.2769 |
| 377 | E | 0.1878 | 0.4918 | 1 | 0.304 |
| 378 | S | 0.2122 | 0.5173 | 1 | 0.3051 |
| 379 | N | 0.2292 | 0.5374 | 1 | 0.3082 |
| 380 | N | 0.2602 | 0.5623 | 1 | 0.3021 |
| 381 | I | 0.2657 | 0.5711 | 1 | 0.3054 |
| 382 | P | 0.2657 | 0.5623 | 1 | 0.2966 |
| 383 | E | 0.2333 | 0.5331 | 1 | 0.2998 |
| 384 | Q | 0.1416 | 0.5331 | 1 | 0.3915 |
| 385 | S | 0.1178 | 0.5008 | 1 | 0.383 |
| 386 | K | 0.1456 | 0.5331 | 1 | 0.3875 |
| 387 | H | 0.1456 | 0.5296 | 1 | 0.384 |
| 388 | S | 0.0991 | 0.4651 | 1 | 0.366 |
| 389 | L | 0.1805 | 0.4703 | 1 | 0.2898 |
| 390 | K | 0.1844 | 0.4825 | 1 | 0.2981 |
| 391 | G | 0.1921 | 0.4825 | 1 | 0.2904 |
| 392 | R | 0.2657 | 0.5583 | 1 | 0.2926 |
| 393 | L | 0.2255 | 0.5229 | 1 | 0.2974 |
| 394 | S | 0.2122 | 0.5084 | 1 | 0.2962 |
| 395 | V | 0.2209 | 0.5126 | 1 | 0.2917 |
| 396 | E | 0.3359 | 0.5229 | 1 | 0.187 |
| 397 | I | 0.2748 | 0.46 | 1 | 0.1852 |
| 398 | L | 0.3053 | 0.4781 | 1 | 0.1728 |
| 399 | E | 0.2865 | 0.4651 | 1 | 0.1786 |
| 400 | Q | 0.3578 | 0.5296 | 1 | 0.1718 |
| 401 | E | 0.2963 | 0.4703 | 1 | 0.174 |
| 402 | D | 0.3311 | 0.4967 | 1 | 0.1656 |
| 403 | K | 0.3762 | 0.5331 | 1 | 0.1569 |
| 404 | R | 0.4458 | 0.5992 | 0 | 0.1534 |
| 405 | E | 0.4703 | 0.5173 | 0 | 0.047 |
| 406 | Q | 0.5008 | 0.5412 | 0 | 0.0404 |
| 407 | I | 0.4203 | 0.4651 | 0 | 0.0448 |
| 408 | P | 0.4556 | 0.5043 | 0 | 0.0487 |
| 409 | R | 0.4879 | 0.5296 | 0 | 0.0417 |
| 410 | D | 0.4967 | 0.5374 | 0 | 0.0407 |
| 411 | V | 0.5043 | 0.5374 | 0 | 0.0331 |
| 412 | P | 0.4203 | 0.4556 | 0 | 0.0353 |
| 413 | P | 0.4037 | 0.4282 | 0 | 0.0245 |
| 414 | S | 0.3806 | 0.4078 | 0 | 0.0272 |
| 415 | F | 0.3939 | 0.4149 | 0 | 0.021 |
| 416 | A | 0.3096 | 0.3311 | 0 | 0.0215 |
| 417 | V | 0.2333 | 0.2483 | 0 | 0.015 |
| 418 | A | 0.3184 | 0.3359 | 0 | 0.0175 |
| 419 | A | 0.208 | 0.2255 | 0 | 0.0175 |
| 420 | D | 0.2333 | 0.2483 | 0 | 0.015 |
| 421 | E | 0.2292 | 0.2432 | 0 | 0.014 |
| 422 | L | 0.2913 | 0.3053 | 0 | 0.014 |
| 423 | K | 0.1921 | 0.1998 | 0 | 0.0077 |
| 424 | M | 0.138 | 0.1495 | 0 | 0.0115 |
| 425 | T | 0.0832 | 0.0935 | 0 | 0.0103 |
| 426 | L | 0.1117 | 0.124 | 0 | 0.0123 |
| 427 | L | 0.1322 | 0.1416 | 0 | 0.0094 |
| 428 | D | 0.1805 | 0.1958 | 0 | 0.0153 |
| 429 | L | 0.1322 | 0.1456 | 0 | 0.0134 |
| 430 | E | 0.0909 | 0.1018 | 0 | 0.0109 |
| 431 | N | 0.0991 | 0.1088 | 0 | 0.0097 |
| 432 | R | 0.0909 | 0.1018 | 0 | 0.0109 |
| 433 | V | 0.1566 | 0.1698 | 0 | 0.0132 |
| 434 | A | 0.1349 | 0.1532 | 0 | 0.0183 |
| 435 | L | 0.1602 | 0.1805 | 0 | 0.0203 |
| 436 | A | 0.1322 | 0.1532 | 0 | 0.021 |
| 437 | Q | 0.1635 | 0.1878 | 0 | 0.0243 |
| 438 | R | 0.1998 | 0.2255 | 0 | 0.0257 |
| 439 | L | 0.1958 | 0.2255 | 0 | 0.0297 |
| 440 | F | 0.1958 | 0.2255 | 0 | 0.0297 |
| 441 | P | 0.1602 | 0.1844 | 0 | 0.0242 |
| 442 | T | 0.1667 | 0.1921 | 0 | 0.0254 |
| 443 | E | 0.1495 | 0.1766 | 0 | 0.0271 |
| 444 | A | 0.1958 | 0.2255 | 0 | 0.0297 |
| 445 | Q | 0.2255 | 0.2602 | 0 | 0.0347 |
| 446 | A | 0.3096 | 0.3399 | 0 | 0.0303 |
| 447 | A | 0.282 | 0.3762 | 0 | 0.0942 |
| 448 | M | 0.2963 | 0.3847 | 0 | 0.0884 |
| 449 | E | 0.2255 | 0.3184 | 0 | 0.0929 |
| 450 | I | 0.2531 | 0.3399 | 0 | 0.0868 |
| 451 | A | 0.282 | 0.3668 | 0 | 0.0848 |
| 452 | E | 0.2531 | 0.3399 | 0 | 0.0868 |
| 453 | M | 0.2558 | 0.3399 | 0 | 0.0841 |
| 454 | K | 0.1732 | 0.2531 | 0 | 0.0799 |
| 455 | G | 0.2167 | 0.3005 | 0 | 0.0838 |
| 456 | T | 0.2385 | 0.3225 | 0 | 0.084 |
| 457 | S | 0.282 | 0.363 | 0 | 0.081 |
| 458 | E | 0.3096 | 0.3885 | 0 | 0.0789 |
| 459 | F | 0.27 | 0.3491 | 0 | 0.0791 |
| 460 | I | 0.2558 | 0.3399 | 0 | 0.0841 |
| 461 | V | 0.3399 | 0.4203 | 0 | 0.0804 |
| 462 | T | 0.3668 | 0.4513 | 0 | 0.0845 |
| 463 | S | 0.3491 | 0.4333 | 0 | 0.0842 |
| 464 | L | 0.3717 | 0.4556 | 0 | 0.0839 |
| 465 | E | 0.3806 | 0.4651 | 0 | 0.0845 |
| 466 | P | 0.3762 | 0.4556 | 0 | 0.0794 |
| 467 | D | 0.4037 | 0.4879 | 0 | 0.0842 |
| 468 | R | 0.4703 | 0.4918 | 0 | 0.0215 |
| 469 | L | 0.3939 | 0.4149 | 0 | 0.021 |
| 470 | T | 0.4651 | 0.4879 | 0 | 0.0228 |
| 471 | G | 0.4556 | 0.4781 | 0 | 0.0225 |
| 472 | T | 0.4967 | 0.5173 | 0 | 0.0206 |
| 473 | K | 0.5253 | 0.5473 | 0 | 0.022 |
| 474 | R | 0.4379 | 0.46 | 0 | 0.0221 |
| 475 | T | 0.5126 | 0.5331 | 0 | 0.0205 |
| 476 | S | 0.4379 | 0.4556 | 0 | 0.0177 |
| 477 | P | 0.3535 | 0.3717 | 0 | 0.0182 |
| 478 | G | 0.3399 | 0.363 | 0 | 0.0231 |
| 479 | V | 0.3399 | 0.3578 | 0 | 0.0179 |
| 480 | K | 0.4078 | 0.4333 | 0 | 0.0255 |
| 481 | I | 0.4203 | 0.4458 | 0 | 0.0255 |
| 482 | A | 0.4149 | 0.4379 | 0 | 0.023 |
| 483 | P | 0.4149 | 0.4379 | 0 | 0.023 |
| 484 | F | 0.3491 | 0.3717 | 0 | 0.0226 |
| 485 | R | 0.3535 | 0.3762 | 0 | 0.0227 |
| 486 | I | 0.3225 | 0.3491 | 0 | 0.0266 |
| 487 | L | 0.2531 | 0.282 | 0 | 0.0289 |
| 488 | E | 0.2255 | 0.2531 | 0 | 0.0276 |
| 489 | E | 0.2209 | 0.2483 | 0 | 0.0274 |
| 490 | H | 0.2786 | 0.3096 | 0 | 0.031 |
| 491 | Q | 0.2167 | 0.2432 | 0 | 0.0265 |
| 492 | S | 0.2865 | 0.3184 | 0 | 0.0319 |
| 493 | R | 0.2209 | 0.2558 | 0 | 0.0349 |
| 494 | L | 0.1921 | 0.2292 | 0 | 0.0371 |
| 495 | K | 0.27 | 0.3146 | 0 | 0.0446 |
| 496 | A | 0.2558 | 0.3053 | 0 | 0.0495 |
| 497 | L | 0.2333 | 0.2865 | 0 | 0.0532 |
| 498 | S | 0.2122 | 0.2657 | 0 | 0.0535 |
| 499 | K | 0.2483 | 0.3053 | 0 | 0.057 |
| 500 | T | 0.2432 | 0.3005 | 0 | 0.0573 |
| 501 | V | 0.1416 | 0.2963 | 0 | 0.1547 |
| 502 | E | 0.1349 | 0.282 | 0 | 0.1471 |
| 503 | L | 0.1205 | 0.27 | 0 | 0.1495 |
| 504 | G | 0.0858 | 0.2122 | 0 | 0.1264 |
| 505 | K | 0.0909 | 0.2255 | 0 | 0.1346 |
| 506 | R | 0.0909 | 0.2255 | 0 | 0.1346 |
| 507 | F | 0.1088 | 0.2602 | 0 | 0.1514 |
| 508 | F | 0.1088 | 0.2602 | 0 | 0.1514 |
| 509 | P | 0.0991 | 0.2483 | 0 | 0.1492 |
| 510 | R | 0.1018 | 0.2531 | 1 | 0.1513 |
| 511 | S | 0.0502 | 0.2558 | 1 | 0.2056 |
| 512 | S | 0.0813 | 0.3263 | 1 | 0.245 |
| 513 | A | 0.0789 | 0.3263 | 1 | 0.2474 |
| 514 | V | 0.0909 | 0.3491 | 1 | 0.2582 |
| 515 | L | 0.0884 | 0.3399 | 1 | 0.2515 |
| 516 | D | 0.0909 | 0.3491 | 1 | 0.2582 |
| 517 | Q | 0.0607 | 0.2963 | 1 | 0.2356 |
| 518 | I | 0.0884 | 0.3491 | 1 | 0.2607 |
| 519 | M | 0.0771 | 0.4333 | 1 | 0.3562 |
| 520 | N | 0.0607 | 0.3992 | 1 | 0.3385 |
| 521 | S | 0.0643 | 0.4116 | 1 | 0.3473 |
| 522 | E | 0.1322 | 0.4149 | 1 | 0.2827 |
| 523 | D | 0.1322 | 0.4203 | 1 | 0.2881 |
| 524 | L | 0.1456 | 0.4379 | 1 | 0.2923 |
| 525 | T | 0.1667 | 0.4703 | 1 | 0.3036 |
| 526 | Q | 0.2385 | 0.5374 | 1 | 0.2989 |
| 527 | L | 0.2209 | 0.5229 | 1 | 0.302 |
| 528 | A | 0.2122 | 0.5043 | 1 | 0.2921 |
| 529 | S | 0.2122 | 0.5126 | 1 | 0.3004 |
| 530 | G | 0.2292 | 0.5331 | 1 | 0.3039 |
| 531 | E | 0.2122 | 0.5126 | 1 | 0.3004 |
| 532 | D | 0.3096 | 0.4967 | 1 | 0.1871 |
| 533 | D | 0.3096 | 0.5008 | 1 | 0.1912 |
| 534 | T | 0.3005 | 0.4918 | 1 | 0.1913 |
| 535 | A | 0.3184 | 0.5043 | 1 | 0.1859 |
| 536 | E | 0.3146 | 0.5008 | 1 | 0.1862 |
| 537 | K | 0.3263 | 0.5043 | 1 | 0.178 |
| 538 | R | 0.3311 | 0.5043 | 1 | 0.1732 |
| 539 | L | 0.363 | 0.5331 | 0 | 0.1701 |
| 540 | Q | 0.4825 | 0.5412 | 0 | 0.0587 |
| 541 | K | 0.4749 | 0.5296 | 0 | 0.0547 |
| 542 | K | 0.4245 | 0.4825 | 0 | 0.058 |
| 543 | Q | 0.4078 | 0.46 | 0 | 0.0522 |
| 544 | R | 0.3847 | 0.4379 | 0 | 0.0532 |
| 545 | Y | 0.3762 | 0.4282 | 0 | 0.052 |
| 546 | M | 0.3535 | 0.3992 | 0 | 0.0457 |
| 547 | E | 0.3456 | 0.3847 | 0 | 0.0391 |
| 548 | I | 0.363 | 0.4037 | 0 | 0.0407 |
| 549 | Q | 0.3668 | 0.4078 | 0 | 0.041 |
| 550 | E | 0.442 | 0.4781 | 0 | 0.0361 |
| 551 | T | 0.4037 | 0.4282 | 0 | 0.0245 |
| 552 | L | 0.4116 | 0.4379 | 0 | 0.0263 |
| 553 | K | 0.3806 | 0.4078 | 0 | 0.0272 |
| 554 | K | 0.3847 | 0.4037 | 0 | 0.019 |
| 555 | A | 0.3992 | 0.4149 | 0 | 0.0157 |
| 556 | F | 0.4513 | 0.4651 | 0 | 0.0138 |
| 557 | S | 0.46 | 0.4703 | 0 | 0.0103 |
| 558 | E | 0.4078 | 0.4149 | 0 | 0.0071 |
| 559 | D | 0.4703 | 0.4781 | 0 | 0.0078 |
| 560 | N | 0.4556 | 0.46 | 0 | 0.0044 |
| 561 | L | 0.442 | 0.4513 | 0 | 0.0093 |
| 562 | E | 0.4513 | 0.4556 | 0 | 0.0043 |
| 563 | L | 0.4967 | 0.5008 | 0 | 0.0041 |
| 564 | G | 0.5084 | 0.5126 | 0 | 0.0042 |
| 565 | N | 0.5229 | 0.5229 | 0 | 0 |
| 566 | S | 0.5331 | 0.5331 | 0 | 0 |
| 567 | S | 0.5846 | 0.5846 | 0 | 0 |
| 568 | L | 0.5846 | 0.5846 | 0 | 0 |
| 569 | T | 0.5802 | 0.5802 | 0 | 0 |
| 570 | D | 0.59 | 0.59 | 0 | 0 |
| 571 | S | 0.5941 | 0.5941 | 0 | 0 |
| 572 | T | 0.6334 | 0.6334 | 0 | 0 |
| 573 | S | 0.6442 | 0.6442 | 0 | 0 |
| 574 | S | 0.6827 | 0.6827 | 0 | 0 |
| 575 | T | 0.6827 | 0.6827 | 0 | 0 |
| 576 | S | 0.687 | 0.687 | 0 | 0 |
| 577 | K | 0.6944 | 0.6944 | 0 | 0 |
| 578 | S | 0.6756 | 0.6756 | 0 | 0 |
| 579 | T | 0.7079 | 0.7079 | 0 | 0 |
| 580 | G | 0.7275 | 0.7275 | 0 | 0 |
| 581 | G | 0.7501 | 0.7501 | 0 | 0 |
| 582 | K | 0.7724 | 0.7724 | 0 | 0 |
| 583 | R | 0.7951 | 0.7951 | 0 | 0 |
| 584 | S | 0.835 | 0.835 | 0 | 0 |
| 585 | N | 0.865 | 0.865 | 0 | 0 |
| 586 | R | 0.8984 | 0.8984 | 0 | 0 |
| 587 | K | 0.9235 | 0.9235 | 0 | 0 |
| 588 | L | 0.9423 | 0.9423 | 0 | 0 |
| 589 | S | 0.9624 | 0.9624 | 0 | 0 |
| 590 | H | 0.9746 | 0.9746 | 0 | 0 |
| 591 | R | 0.9831 | 0.9831 | 0 | 0 |
| 592 | R | 0.9899 | 0.9899 | 0 | 0 |
| 593 | R | 0.996 | 0.996 | 0 | 0 |
| >AtNPR2 | | | | | |
| (AT4G26120) | | | | | |
| 1 | M | 0.928 | 0.928 | 0 | 0 |
| 2 | A | 0.8556 | 0.8556 | 0 | 0 |
| 3 | T | 0.8158 | 0.8158 | 0 | 0 |
| 4 | T | 0.7772 | 0.7772 | 0 | 0 |
| 5 | T | 0.7317 | 0.7317 | 0 | 0 |
| 6 | T | 0.6293 | 0.6293 | 0 | 0 |
| 7 | T | 0.6174 | 0.6174 | 0 | 0 |
| 8 | T | 0.5084 | 0.5084 | 0 | 0 |
| 9 | T | 0.4556 | 0.4556 | 0 | 0 |
| 10 | A | 0.4149 | 0.4149 | 0 | 0 |
| 11 | R | 0.3717 | 0.3717 | 0 | 0 |
| 12 | F | 0.3806 | 0.3806 | 0 | 0 |
| 13 | S | 0.4078 | 0.4078 | 0 | 0 |
| 14 | D | 0.4149 | 0.4149 | 0 | 0 |
| 15 | S | 0.4149 | 0.4149 | 0 | 0 |
| 16 | Y | 0.3456 | 0.3456 | 0 | 0 |
| 17 | E | 0.2786 | 0.2786 | 0 | 0 |
| 18 | F | 0.2602 | 0.2602 | 0 | 0 |
| 19 | S | 0.2333 | 0.2385 | 0 | 0.0052 |
| 20 | N | 0.2657 | 0.2657 | 0 | 0 |
| 21 | T | 0.2865 | 0.2865 | 0 | 0 |
| 22 | S | 0.2748 | 0.2748 | 0 | 0 |
| 23 | G | 0.2748 | 0.2748 | 0 | 0 |
| 24 | N | 0.2963 | 0.3005 | 0 | 0.0042 |
| 25 | S | 0.2167 | 0.2209 | 0 | 0.0042 |
| 26 | F | 0.2558 | 0.2602 | 0 | 0.0044 |
| 27 | F | 0.3225 | 0.3311 | 0 | 0.0086 |
| 28 | A | 0.3225 | 0.3263 | 0 | 0.0038 |
| 29 | A | 0.3096 | 0.3225 | 0 | 0.0129 |
| 30 | E | 0.2558 | 0.27 | 0 | 0.0142 |
| 31 | S | 0.2531 | 0.2657 | 0 | 0.0126 |
| 32 | S | 0.282 | 0.2963 | 0 | 0.0143 |
| 33 | L | 0.3225 | 0.3359 | 0 | 0.0134 |
| 34 | D | 0.3456 | 0.3578 | 0 | 0.0122 |
| 35 | Y | 0.2786 | 0.2963 | 0 | 0.0177 |
| 36 | P | 0.2786 | 0.2963 | 0 | 0.0177 |
| 37 | T | 0.3225 | 0.3399 | 0 | 0.0174 |
| 38 | E | 0.3146 | 0.3359 | 0 | 0.0213 |
| 39 | F | 0.3184 | 0.3399 | 0 | 0.0215 |
| 40 | L | 0.27 | 0.2913 | 0 | 0.0213 |
| 41 | T | 0.1766 | 0.1958 | 0 | 0.0192 |
| 42 | P | 0.1766 | 0.1998 | 0 | 0.0232 |
| 43 | P | 0.1878 | 0.208 | 0 | 0.0202 |
| 44 | E | 0.1958 | 0.27 | 0 | 0.0742 |
| 45 | V | 0.1292 | 0.1878 | 0 | 0.0586 |
| 46 | S | 0.208 | 0.2786 | 0 | 0.0706 |
| 47 | A | 0.1766 | 0.2432 | 0 | 0.0666 |
| 48 | L | 0.1322 | 0.1878 | 0 | 0.0556 |
| 49 | K | 0.066 | 0.1041 | 0 | 0.0381 |
| 50 | L | 0.115 | 0.1698 | 0 | 0.0548 |
| 51 | L | 0.1602 | 0.2167 | 0 | 0.0565 |
| 52 | S | 0.1844 | 0.2531 | 0 | 0.0687 |
| 53 | N | 0.1732 | 0.2385 | 0 | 0.0653 |
| 54 | S | 0.1495 | 0.2122 | 0 | 0.0627 |
| 55 | L | 0.0723 | 0.115 | 0 | 0.0427 |
| 56 | E | 0.066 | 0.106 | 0 | 0.04 |
| 57 | S | 0.0677 | 0.1117 | 0 | 0.044 |
| 58 | V | 0.0909 | 0.138 | 0 | 0.0471 |
| 59 | F | 0.1117 | 0.1698 | 0 | 0.0581 |
| 60 | D | 0.1205 | 0.1805 | 0 | 0.06 |
| 61 | S | 0.115 | 0.1732 | 0 | 0.0582 |
| 62 | P | 0.1178 | 0.1766 | 0 | 0.0588 |
| 63 | E | 0.0677 | 0.1178 | 0 | 0.0501 |
| 64 | T | 0.0526 | 0.0935 | 0 | 0.0409 |
| 65 | F | 0.0771 | 0.0991 | 0 | 0.022 |
| 66 | Y | 0.115 | 0.1495 | 0 | 0.0345 |
| 67 | S | 0.0991 | 0.1292 | 0 | 0.0301 |
| 68 | D | 0.1088 | 0.1456 | 0 | 0.0368 |
| 69 | A | 0.1041 | 0.138 | 0 | 0.0339 |
| 70 | K | 0.1667 | 0.208 | 0 | 0.0413 |
| 71 | L | 0.0935 | 0.124 | 0 | 0.0305 |
| 72 | V | 0.0991 | 0.1322 | 0 | 0.0331 |
| 73 | L | 0.0832 | 0.115 | 0 | 0.0318 |
| 74 | A | 0.0502 | 0.1018 | 0 | 0.0516 |
| 75 | G | 0.0327 | 0.0643 | 0 | 0.0316 |
| 76 | G | 0.0405 | 0.0771 | 0 | 0.0366 |
| 77 | R | 0.0621 | 0.1178 | 0 | 0.0557 |
| 78 | E | 0.0502 | 0.0965 | 0 | 0.0463 |
| 79 | V | 0.0502 | 0.0965 | 0 | 0.0463 |
| 80 | S | 0.0376 | 0.0677 | 0 | 0.0301 |
| 81 | F | 0.0464 | 0.0884 | 0 | 0.042 |
| 82 | H | 0.0526 | 0.0965 | 0 | 0.0439 |
| 83 | R | 0.0464 | 0.0858 | 0 | 0.0394 |
| 84 | S | 0.0832 | 0.1416 | 0 | 0.0584 |
| 85 | I | 0.106 | 0.1698 | 0 | 0.0638 |
| 86 | L | 0.0884 | 0.1416 | 0 | 0.0532 |
| 87 | S | 0.0526 | 0.0909 | 0 | 0.0383 |
| 88 | A | 0.0455 | 0.0744 | 0 | 0.0289 |
| 89 | R | 0.0405 | 0.0677 | 0 | 0.0272 |
| 90 | I | 0.0425 | 0.0701 | 0 | 0.0276 |
| 91 | P | 0.0425 | 0.0701 | 0 | 0.0276 |
| 92 | V | 0.0771 | 0.1266 | 0 | 0.0495 |
| 93 | F | 0.0771 | 0.124 | 0 | 0.0469 |
| 94 | K | 0.0723 | 0.1178 | 0 | 0.0455 |
| 95 | S | 0.106 | 0.1205 | 0 | 0.0145 |
| 96 | A | 0.1532 | 0.1698 | 0 | 0.0166 |
| 97 | L | 0.208 | 0.2292 | 0 | 0.0212 |
| 98 | A | 0.2041 | 0.2255 | 0 | 0.0214 |
| 99 | T | 0.1667 | 0.1844 | 0 | 0.0177 |
| 100 | V | 0.1566 | 0.1698 | 0 | 0.0132 |
| 101 | K | 0.1532 | 0.1698 | 0 | 0.0166 |
| 102 | E | 0.1292 | 0.1456 | 0 | 0.0164 |
| 103 | Q | 0.1178 | 0.1322 | 0 | 0.0144 |
| 104 | K | 0.1844 | 0.1998 | 0 | 0.0154 |
| 105 | S | 0.1878 | 0.2041 | 0 | 0.0163 |
| 106 | S | 0.1205 | 0.1322 | 0 | 0.0117 |
| 107 | T | 0.1178 | 0.1266 | 0 | 0.0088 |
| 108 | T | 0.1805 | 0.1921 | 0 | 0.0116 |
| 109 | V | 0.2167 | 0.2292 | 0 | 0.0125 |
| 110 | K | 0.1602 | 0.1667 | 0 | 0.0065 |
| 111 | L | 0.2209 | 0.2292 | 0 | 0.0083 |
| 112 | Q | 0.1667 | 0.1732 | 0 | 0.0065 |
| 113 | L | 0.1635 | 0.1698 | 0 | 0.0063 |
| 114 | K | 0.0991 | 0.1018 | 0 | 0.0027 |
| 115 | E | 0.1088 | 0.1117 | 0 | 0.0029 |
| 116 | I | 0.1088 | 0.1117 | 0 | 0.0029 |
| 117 | A | 0.0789 | 0.0813 | 0 | 0.0024 |
| 118 | R | 0.0567 | 0.0587 | 0 | 0.002 |
| 119 | D | 0.0478 | 0.0502 | 0 | 0.0024 |
| 120 | Y | 0.0554 | 0.0607 | 0 | 0.0053 |
| 121 | E | 0.0387 | 0.0425 | 0 | 0.0038 |
| 122 | V | 0.0542 | 0.0621 | 0 | 0.0079 |
| 123 | G | 0.0336 | 0.0405 | 0 | 0.0069 |
| 124 | F | 0.0405 | 0.049 | 0 | 0.0085 |
| 125 | D | 0.0268 | 0.0327 | 0 | 0.0059 |
| 126 | S | 0.0252 | 0.0316 | 0 | 0.0064 |
| 127 | V | 0.0414 | 0.0554 | 0 | 0.014 |
| 128 | V | 0.0514 | 0.0701 | 0 | 0.0187 |
| 129 | A | 0.035 | 0.0502 | 0 | 0.0152 |
| 130 | V | 0.0308 | 0.0455 | 0 | 0.0147 |
| 131 | L | 0.049 | 0.0677 | 0 | 0.0187 |
| 132 | A | 0.0542 | 0.0789 | 0 | 0.0247 |
| 133 | Y | 0.0935 | 0.1322 | 0 | 0.0387 |
| 134 | V | 0.0858 | 0.1266 | 0 | 0.0408 |
| 135 | Y | 0.138 | 0.1958 | 0 | 0.0578 |
| 136 | S | 0.1018 | 0.1566 | 0 | 0.0548 |
| 137 | G | 0.1018 | 0.1566 | 0 | 0.0548 |
| 138 | R | 0.1088 | 0.1698 | 0 | 0.061 |
| 139 | V | 0.0677 | 0.2122 | 0 | 0.1445 |
| 140 | R | 0.0542 | 0.1805 | 0 | 0.1263 |
| 141 | S | 0.0813 | 0.2385 | 1 | 0.1572 |
| 142 | P | 0.1292 | 0.3184 | 1 | 0.1892 |
| 143 | P | 0.1667 | 0.363 | 1 | 0.1963 |
| 144 | K | 0.1322 | 0.4203 | 1 | 0.2881 |
| 145 | G | 0.1041 | 0.4749 | 1 | 0.3708 |
| 146 | A | 0.138 | 0.5374 | 1 | 0.3994 |
| 147 | S | 0.0935 | 0.4749 | 1 | 0.3814 |
| 148 | A | 0.0771 | 0.4379 | 1 | 0.3608 |
| 149 | S | 0.0336 | 0.4379 | 1 | 0.4043 |
| 150 | V | 0.0464 | 0.4825 | 1 | 0.4361 |
| 151 | D | 0.049 | 0.4879 | 1 | 0.4389 |
| 152 | D | 0.0464 | 0.4825 | 1 | 0.4361 |
| 153 | D | 0.0259 | 0.3885 | 1 | 0.3626 |
| 154 | S | 0.0245 | 0.3806 | 1 | 0.3561 |
| 155 | S | 0.0116 | 0.2865 | 1 | 0.2749 |
| 156 | H | 0.009 | 0.2531 | 1 | 0.2441 |
| 157 | V | 0.0078 | 0.2167 | 1 | 0.2089 |
| 158 | A | 0.0082 | 0.2333 | 1 | 0.2251 |
| 159 | S | 0.0069 | 0.1998 | 0 | 0.1929 |
| 160 | R | 0.0082 | 0.1322 | 0 | 0.124 |
| 161 | S | 0.0078 | 0.1266 | 0 | 0.1188 |
| 162 | K | 0.0029 | 0.0621 | 0 | 0.0592 |
| 163 | V | 0.0028 | 0.0567 | 0 | 0.0539 |
| 164 | D | 0.0008 | 0.0259 | 0 | 0.0251 |
| 165 | F | 0.0009 | 0.0144 | 0 | 0.0135 |
| 166 | M | 0.0009 | 0.007 | 0 | 0.0061 |
| 167 | V | 0.0009 | 0.007 | 0 | 0.0061 |
| 168 | E | 0.0007 | 0.0053 | 0 | 0.0046 |
| 169 | V | 0.0008 | 0.0067 | 0 | 0.0059 |
| 170 | L | 0.0035 | 0.0074 | 0 | 0.0039 |
| 171 | Y | 0.001 | 0.0028 | 0 | 0.0018 |
| 172 | L | 0.0006 | 0.0009 | 0 | 0.0003 |
| 173 | S | 0.0006 | 0.0009 | 0 | 0.0003 |
| 174 | F | 0.0005 | 0.0007 | 0 | 0.0002 |
| 175 | V | 0.0003 | 0.0004 | 0 | 0.0001 |
| 176 | F | 0.0005 | 0.0008 | 0 | 0.0003 |
| 177 | Q | 0.0006 | 0.0008 | 0 | 0.0002 |
| 178 | I | 0.0008 | 0.0013 | 0 | 0.0005 |
| 179 | Q | 0.0004 | 0.0006 | 0 | 0.0002 |
| 180 | E | 0.0003 | 0.0005 | 0 | 0.0002 |
| 181 | L | 0.0006 | 0.0008 | 0 | 0.0002 |
| 182 | V | 0.0005 | 0.0006 | 0 | 1E−04 |
| 183 | T | 0.0005 | 0.0006 | 0 | 1E−04 |
| 184 | L | 0.0005 | 0.0007 | 0 | 0.0002 |
| 185 | Y | 0.0008 | 0.0012 | 0 | 0.0004 |
| 186 | E | 0.0008 | 0.0009 | 0 | 1E−04 |
| 187 | R | 0.0009 | 0.0012 | 0 | 0.0003 |
| 188 | Q | 0.0006 | 0.0007 | 0 | 0.0001 |
| 189 | F | 0.0012 | 0.0022 | 0 | 0.001 |
| 190 | L | 0.0013 | 0.0026 | 0 | 0.0013 |
| 191 | E | 0.0007 | 0.0007 | 0 | 0 |
| 192 | I | 0.0007 | 0.0008 | 0 | 0.0001 |
| 193 | V | 0.0007 | 0.0008 | 0 | 0.0001 |
| 194 | D | 0.0005 | 0.0005 | 0 | 0 |
| 195 | K | 0.0005 | 0.0006 | 0 | 1E−04 |
| 196 | V | 0.0007 | 0.0008 | 0 | 0.0001 |
| 197 | V | 0.0004 | 0.0004 | 0 | 0 |
| 198 | V | 0.0004 | 0.0005 | 0 | 0.0001 |
| 199 | E | 0.0004 | 0.0005 | 0 | 0.0001 |
| 200 | D | 0.0005 | 0.0006 | 0 | 1E−04 |
| 201 | I | 0.0006 | 0.0009 | 0 | 0.0003 |
| 202 | L | 0.0006 | 0.0009 | 0 | 0.0003 |
| 203 | V | 0.0009 | 0.0031 | 0 | 0.0022 |
| 204 | I | 0.0027 | 0.0067 | 0 | 0.004 |
| 205 | F | 0.0009 | 0.0029 | 0 | 0.002 |
| 206 | K | 0.0009 | 0.0029 | 0 | 0.002 |
| 207 | L | 0.0022 | 0.0059 | 0 | 0.0037 |
| 208 | D | 0.0018 | 0.0055 | 0 | 0.0037 |
| 209 | T | 0.0013 | 0.0053 | 0 | 0.004 |
| 210 | L | 0.0013 | 0.0052 | 0 | 0.0039 |
| 211 | S | 0.0012 | 0.0042 | 0 | 0.003 |
| 212 | G | 0.0022 | 0.009 | 0 | 0.0068 |
| 213 | T | 0.0024 | 0.009 | 0 | 0.0066 |
| 214 | T | 0.0052 | 0.0182 | 0 | 0.013 |
| 215 | Y | 0.0053 | 0.0194 | 0 | 0.0141 |
| 216 | K | 0.0059 | 0.0212 | 0 | 0.0153 |
| 217 | K | 0.0031 | 0.0121 | 0 | 0.009 |
| 218 | L | 0.0074 | 0.0268 | 0 | 0.0194 |
| 219 | L | 0.007 | 0.0259 | 0 | 0.0189 |
| 220 | D | 0.0078 | 0.0274 | 0 | 0.0196 |
| 221 | R | 0.0082 | 0.0304 | 0 | 0.0222 |
| 222 | S | 0.0141 | 0.0316 | 0 | 0.0175 |
| 223 | I | 0.0074 | 0.015 | 0 | 0.0076 |
| 224 | E | 0.0042 | 0.009 | 0 | 0.0048 |
| 225 | I | 0.0052 | 0.0099 | 0 | 0.0047 |
| 226 | I | 0.0044 | 0.0094 | 0 | 0.005 |
| 227 | V | 0.0059 | 0.0118 | 0 | 0.0059 |
| 228 | K | 0.0062 | 0.0124 | 0 | 0.0062 |
| 229 | S | 0.0121 | 0.0274 | 0 | 0.0153 |
| 230 | D | 0.0121 | 0.0274 | 0 | 0.0153 |
| 231 | I | 0.015 | 0.0327 | 0 | 0.0177 |
| 232 | E | 0.0157 | 0.0363 | 0 | 0.0206 |
| 233 | L | 0.0279 | 0.0376 | 0 | 0.0097 |
| 234 | V | 0.0308 | 0.0414 | 0 | 0.0106 |
| 235 | S | 0.0144 | 0.0218 | 0 | 0.0074 |
| 236 | L | 0.0308 | 0.0425 | 0 | 0.0117 |
| 237 | E | 0.0587 | 0.0858 | 0 | 0.0271 |
| 238 | K | 0.0554 | 0.0789 | 0 | 0.0235 |
| 239 | S | 0.0336 | 0.0464 | 0 | 0.0128 |
| 240 | L | 0.035 | 0.049 | 0 | 0.014 |
| 241 | P | 0.0182 | 0.0274 | 0 | 0.0092 |
| 242 | Q | 0.035 | 0.049 | 0 | 0.014 |
| 243 | H | 0.035 | 0.049 | 0 | 0.014 |
| 244 | I | 0.0554 | 0.0771 | 0 | 0.0217 |
| 245 | F | 0.0514 | 0.0701 | 0 | 0.0187 |
| 246 | K | 0.0376 | 0.0701 | 0 | 0.0325 |
| 247 | Q | 0.0387 | 0.0701 | 0 | 0.0314 |
| 248 | I | 0.0387 | 0.0701 | 0 | 0.0314 |
| 249 | I | 0.049 | 0.0909 | 0 | 0.0419 |
| 250 | D | 0.0587 | 0.1117 | 0 | 0.053 |
| 251 | I | 0.1088 | 0.1805 | 0 | 0.0717 |
| 252 | R | 0.0502 | 0.0909 | 0 | 0.0407 |
| 253 | E | 0.0526 | 0.0935 | 0 | 0.0409 |
| 254 | A | 0.0514 | 0.0884 | 0 | 0.037 |
| 255 | L | 0.0909 | 0.1495 | 0 | 0.0586 |
| 256 | S | 0.1041 | 0.1635 | 0 | 0.0594 |
| 257 | L | 0.1041 | 0.1602 | 0 | 0.0561 |
| 258 | E | 0.1041 | 0.1566 | 0 | 0.0525 |
| 259 | P | 0.106 | 0.1602 | 0 | 0.0542 |
| 260 | P | 0.1178 | 0.1766 | 0 | 0.0588 |
| 261 | K | 0.106 | 0.1635 | 0 | 0.0575 |
| 262 | L | 0.1495 | 0.2167 | 0 | 0.0672 |
| 263 | E | 0.0909 | 0.1456 | 0 | 0.0547 |
| 264 | R | 0.0909 | 0.1416 | 0 | 0.0507 |
| 265 | H | 0.1088 | 0.1667 | 0 | 0.0579 |
| 266 | V | 0.1766 | 0.2531 | 0 | 0.0765 |
| 267 | K | 0.2292 | 0.2558 | 0 | 0.0266 |
| 268 | N | 0.2483 | 0.2748 | 0 | 0.0265 |
| 269 | I | 0.2558 | 0.282 | 0 | 0.0262 |
| 270 | Y | 0.1456 | 0.1667 | 0 | 0.0211 |
| 271 | K | 0.0813 | 0.0991 | 0 | 0.0178 |
| 272 | A | 0.0789 | 0.0965 | 0 | 0.0176 |
| 273 | L | 0.1117 | 0.1292 | 0 | 0.0175 |
| 274 | D | 0.0621 | 0.0771 | 0 | 0.015 |
| 275 | S | 0.0405 | 0.049 | 0 | 0.0085 |
| 276 | D | 0.0226 | 0.0274 | 0 | 0.0048 |
| 277 | D | 0.0395 | 0.0464 | 0 | 0.0069 |
| 278 | V | 0.0387 | 0.0464 | 0 | 0.0077 |
| 279 | E | 0.0363 | 0.0455 | 0 | 0.0092 |
| 280 | L | 0.0607 | 0.0771 | 0 | 0.0164 |
| 281 | V | 0.0991 | 0.1205 | 0 | 0.0214 |
| 282 | K | 0.0542 | 0.0677 | 0 | 0.0135 |
| 283 | M | 0.0677 | 0.0858 | 0 | 0.0181 |
| 284 | L | 0.1349 | 0.1602 | 0 | 0.0253 |
| 285 | L | 0.106 | 0.1266 | 0 | 0.0206 |
| 286 | L | 0.0587 | 0.0771 | 0 | 0.0184 |
| 287 | E | 0.0464 | 0.0587 | 0 | 0.0123 |
| 288 | G | 0.0231 | 0.0297 | 0 | 0.0066 |
| 289 | H | 0.035 | 0.0464 | 0 | 0.0114 |
| 290 | T | 0.0137 | 0.0194 | 0 | 0.0057 |
| 291 | N | 0.0226 | 0.0297 | 0 | 0.0071 |
| 292 | L | 0.0157 | 0.0226 | 0 | 0.0069 |
| 293 | D | 0.0124 | 0.0167 | 0 | 0.0043 |
| 294 | E | 0.016 | 0.0226 | 0 | 0.0066 |
| 295 | A | 0.016 | 0.0387 | 0 | 0.0227 |
| 296 | Y | 0.0231 | 0.0502 | 0 | 0.0271 |
| 297 | A | 0.0268 | 0.0542 | 0 | 0.0274 |
| 298 | L | 0.0231 | 0.0502 | 0 | 0.0271 |
| 299 | H | 0.0252 | 0.0514 | 0 | 0.0262 |
| 300 | F | 0.0194 | 0.0414 | 0 | 0.022 |
| 301 | A | 0.0102 | 0.0259 | 0 | 0.0157 |
| 302 | I | 0.0109 | 0.0259 | 0 | 0.015 |
| 303 | A | 0.0121 | 0.0279 | 0 | 0.0158 |
| 304 | H | 0.0069 | 0.0137 | 0 | 0.0068 |
| 305 | S | 0.0066 | 0.0128 | 0 | 0.0062 |
| 306 | A | 0.004 | 0.0086 | 0 | 0.0046 |
| 307 | V | 0.0081 | 0.0173 | 0 | 0.0092 |
| 308 | K | 0.0059 | 0.0118 | 0 | 0.0059 |
| 309 | T | 0.0084 | 0.0194 | 0 | 0.011 |
| 310 | A | 0.0087 | 0.02 | 0 | 0.0113 |
| 311 | Y | 0.0116 | 0.0259 | 0 | 0.0143 |
| 312 | D | 0.0144 | 0.0316 | 0 | 0.0172 |
| 313 | L | 0.0144 | 0.0316 | 0 | 0.0172 |
| 314 | L | 0.0194 | 0.0405 | 0 | 0.0211 |
| 315 | E | 0.0212 | 0.0425 | 0 | 0.0213 |
| 316 | L | 0.0425 | 0.0502 | 0 | 0.0077 |
| 317 | E | 0.0526 | 0.0643 | 0 | 0.0117 |
| 318 | L | 0.0832 | 0.1018 | 0 | 0.0186 |
| 319 | A | 0.0478 | 0.0587 | 0 | 0.0109 |
| 320 | D | 0.049 | 0.0587 | 0 | 0.0097 |
| 321 | V | 0.0395 | 0.0464 | 0 | 0.0069 |
| 322 | N | 0.0387 | 0.0455 | 0 | 0.0068 |
| 323 | L | 0.0376 | 0.0441 | 0 | 0.0065 |
| 324 | R | 0.0405 | 0.0464 | 0 | 0.0059 |
| 325 | N | 0.0554 | 0.0643 | 0 | 0.0089 |
| 326 | P | 0.0414 | 0.0478 | 0 | 0.0064 |
| 327 | R | 0.0587 | 0.0677 | 0 | 0.009 |
| 328 | G | 0.0542 | 0.0621 | 0 | 0.0079 |
| 329 | Y | 0.0991 | 0.1088 | 0 | 0.0097 |
| 330 | T | 0.1292 | 0.1416 | 0 | 0.0124 |
| 331 | V | 0.1532 | 0.1635 | 0 | 0.0103 |
| 332 | L | 0.2209 | 0.2385 | 0 | 0.0176 |
| 333 | H | 0.1416 | 0.1532 | 0 | 0.0116 |
| 334 | V | 0.1532 | 0.1635 | 0 | 0.0103 |
| 335 | A | 0.0909 | 0.0965 | 0 | 0.0056 |
| 336 | A | 0.0935 | 0.0965 | 0 | 0.003 |
| 337 | M | 0.0455 | 0.0464 | 0 | 0.0009 |
| 338 | R | 0.0259 | 0.0268 | 0 | 0.0009 |
| 339 | K | 0.0212 | 0.0218 | 0 | 0.0006 |
| 340 | E | 0.0395 | 0.0405 | 0 | 0.001 |
| 341 | P | 0.0395 | 0.0395 | 0 | 0 |
| 342 | K | 0.049 | 0.049 | 0 | 0 |
| 343 | L | 0.0884 | 0.0884 | 0 | 0 |
| 344 | I | 0.049 | 0.049 | 0 | 0 |
| 345 | I | 0.0464 | 0.0464 | 0 | 0 |
| 346 | S | 0.0554 | 0.0554 | 0 | 0 |
| 347 | L | 0.0677 | 0.0677 | 0 | 0 |
| 348 | L | 0.0813 | 0.0813 | 0 | 0 |
| 349 | M | 0.0502 | 0.0502 | 0 | 0 |
| 350 | K | 0.049 | 0.049 | 0 | 0 |
| 351 | G | 0.0455 | 0.0455 | 0 | 0 |
| 352 | A | 0.0387 | 0.0387 | 0 | 0 |
| 353 | N | 0.0376 | 0.0376 | 0 | 0 |
| 354 | I | 0.0542 | 0.0542 | 0 | 0 |
| 355 | L | 0.0643 | 0.0643 | 0 | 0 |
| 356 | D | 0.0832 | 0.0832 | 0 | 0 |
| 357 | T | 0.049 | 0.049 | 0 | 0 |
| 358 | T | 0.0567 | 0.0567 | 0 | 0 |
| 359 | L | 0.0935 | 0.0965 | 0 | 0.003 |
| 360 | D | 0.115 | 0.1178 | 0 | 0.0028 |
| 361 | G | 0.0701 | 0.0723 | 0 | 0.0022 |
| 362 | R | 0.0677 | 0.0701 | 0 | 0.0024 |
| 363 | T | 0.0813 | 0.0858 | 0 | 0.0045 |
| 364 | A | 0.066 | 0.0701 | 0 | 0.0041 |
| 365 | L | 0.1178 | 0.124 | 0 | 0.0062 |
| 366 | V | 0.1805 | 0.1878 | 0 | 0.0073 |
| 367 | I | 0.1322 | 0.1416 | 0 | 0.0094 |
| 368 | V | 0.1266 | 0.1349 | 0 | 0.0083 |
| 369 | K | 0.1266 | 0.138 | 0 | 0.0114 |
| 370 | R | 0.1921 | 0.2041 | 0 | 0.012 |
| 371 | L | 0.1878 | 0.2041 | 0 | 0.0163 |
| 372 | T | 0.1998 | 0.2122 | 0 | 0.0124 |
| 373 | K | 0.1958 | 0.2122 | 0 | 0.0164 |
| 374 | A | 0.2041 | 0.2209 | 0 | 0.0168 |
| 375 | D | 0.2255 | 0.2432 | 0 | 0.0177 |
| 376 | D | 0.3263 | 0.3456 | 0 | 0.0193 |
| 377 | Y | 0.3806 | 0.4037 | 0 | 0.0231 |
| 378 | K | 0.3847 | 0.4116 | 0 | 0.0269 |
| 379 | T | 0.4333 | 0.4556 | 0 | 0.0223 |
| 380 | S | 0.4458 | 0.4703 | 0 | 0.0245 |
| 381 | T | 0.4458 | 0.4749 | 0 | 0.0291 |
| 382 | E | 0.4513 | 0.4781 | 0 | 0.0268 |
| 383 | D | 0.4037 | 0.4825 | 0 | 0.0788 |
| 384 | G | 0.3456 | 0.4245 | 0 | 0.0789 |
| 385 | T | 0.3885 | 0.4703 | 0 | 0.0818 |
| 386 | P | 0.3399 | 0.4203 | 0 | 0.0804 |
| 387 | S | 0.2865 | 0.3668 | 0 | 0.0803 |
| 388 | L | 0.3717 | 0.4458 | 0 | 0.0741 |
| 389 | K | 0.3762 | 0.46 | 0 | 0.0838 |
| 390 | G | 0.3992 | 0.4781 | 0 | 0.0789 |
| 391 | G | 0.4037 | 0.4918 | 0 | 0.0881 |
| 392 | L | 0.3992 | 0.4825 | 0 | 0.0833 |
| 393 | S | 0.3184 | 0.4037 | 0 | 0.0853 |
| 394 | I | 0.3311 | 0.4203 | 0 | 0.0892 |
| 395 | E | 0.2602 | 0.3456 | 0 | 0.0854 |
| 396 | V | 0.1958 | 0.282 | 0 | 0.0862 |
| 397 | L | 0.1732 | 0.2531 | 0 | 0.0799 |
| 398 | E | 0.1921 | 0.2786 | 0 | 0.0865 |
| 399 | H | 0.1805 | 0.2602 | 0 | 0.0797 |
| 400 | E | 0.1998 | 0.282 | 0 | 0.0822 |
| 401 | Q | 0.1698 | 0.2483 | 0 | 0.0785 |
| 402 | K | 0.1698 | 0.2432 | 0 | 0.0734 |
| 403 | L | 0.1495 | 0.2209 | 0 | 0.0714 |
| 404 | E | 0.1998 | 0.2209 | 0 | 0.0211 |
| 405 | Y | 0.1732 | 0.1958 | 0 | 0.0226 |
| 406 | L | 0.1805 | 0.2041 | 0 | 0.0236 |
| 407 | S | 0.1532 | 0.1732 | 0 | 0.02 |
| 408 | P | 0.2041 | 0.2292 | 0 | 0.0251 |
| 409 | I | 0.2167 | 0.2385 | 0 | 0.0218 |
| 410 | E | 0.2292 | 0.2531 | 0 | 0.0239 |
| 411 | A | 0.2255 | 0.2432 | 0 | 0.0177 |
| 412 | S | 0.1416 | 0.1566 | 0 | 0.015 |
| 413 | L | 0.1416 | 0.1532 | 0 | 0.0116 |
| 414 | S | 0.1635 | 0.1732 | 0 | 0.0097 |
| 415 | L | 0.1416 | 0.1495 | 0 | 0.0079 |
| 416 | P | 0.1292 | 0.138 | 0 | 0.0088 |
| 417 | V | 0.124 | 0.1292 | 0 | 0.0052 |
| 418 | T | 0.0744 | 0.0789 | 0 | 0.0045 |
| 419 | P | 0.0387 | 0.0395 | 0 | 0.0008 |
| 420 | E | 0.0723 | 0.0771 | 0 | 0.0048 |
| 421 | E | 0.0677 | 0.0723 | 0 | 0.0046 |
| 422 | L | 0.0789 | 0.0832 | 0 | 0.0043 |
| 423 | R | 0.049 | 0.0514 | 0 | 0.0024 |
| 424 | M | 0.0723 | 0.0771 | 0 | 0.0048 |
| 425 | R | 0.0441 | 0.0464 | 0 | 0.0023 |
| 426 | L | 0.066 | 0.0701 | 0 | 0.0041 |
| 427 | L | 0.0514 | 0.0542 | 0 | 0.0028 |
| 428 | Y | 0.0526 | 0.0567 | 0 | 0.0041 |
| 429 | Y | 0.0316 | 0.035 | 0 | 0.0034 |
| 430 | E | 0.0137 | 0.015 | 0 | 0.0013 |
| 431 | N | 0.015 | 0.0167 | 0 | 0.0017 |
| 432 | R | 0.0083 | 0.009 | 0 | 0.0007 |
| 433 | V | 0.0173 | 0.02 | 0 | 0.0027 |
| 434 | A | 0.0212 | 0.0245 | 0 | 0.0033 |
| 435 | L | 0.0304 | 0.0336 | 0 | 0.0032 |
| 436 | A | 0.0336 | 0.0387 | 0 | 0.0051 |
| 437 | R | 0.0376 | 0.0425 | 0 | 0.0049 |
| 438 | L | 0.0643 | 0.0771 | 0 | 0.0128 |
| 439 | L | 0.1088 | 0.1266 | 0 | 0.0178 |
| 440 | F | 0.1088 | 0.1266 | 0 | 0.0178 |
| 441 | P | 0.0858 | 0.1041 | 0 | 0.0183 |
| 442 | V | 0.0771 | 0.0935 | 0 | 0.0164 |
| 443 | E | 0.0526 | 0.0643 | 0 | 0.0117 |
| 444 | T | 0.0935 | 0.1117 | 0 | 0.0182 |
| 445 | E | 0.1205 | 0.1416 | 0 | 0.0211 |
| 446 | T | 0.1844 | 0.208 | 0 | 0.0236 |
| 447 | V | 0.1566 | 0.2385 | 0 | 0.0819 |
| 448 | Q | 0.1805 | 0.27 | 0 | 0.0895 |
| 449 | G | 0.1766 | 0.2657 | 0 | 0.0891 |
| 450 | I | 0.2483 | 0.3359 | 0 | 0.0876 |
| 451 | A | 0.3096 | 0.4037 | 0 | 0.0941 |
| 452 | K | 0.2865 | 0.3806 | 0 | 0.0941 |
| 453 | L | 0.363 | 0.4513 | 0 | 0.0883 |
| 454 | E | 0.282 | 0.3717 | 0 | 0.0897 |
| 455 | E | 0.2963 | 0.3847 | 0 | 0.0884 |
| 456 | T | 0.3146 | 0.4037 | 0 | 0.0891 |
| 457 | S | 0.3184 | 0.4078 | 0 | 0.0894 |
| 458 | E | 0.3939 | 0.4879 | 0 | 0.094 |
| 459 | F | 0.3847 | 0.4825 | 0 | 0.0978 |
| 460 | T | 0.3184 | 0.4116 | 0 | 0.0932 |
| 461 | A | 0.4037 | 0.5008 | 0 | 0.0971 |
| 462 | S | 0.442 | 0.5374 | 0 | 0.0954 |
| 463 | S | 0.4556 | 0.5473 | 0 | 0.0917 |
| 464 | L | 0.5229 | 0.6124 | 0 | 0.0895 |
| 465 | E | 0.5043 | 0.5992 | 0 | 0.0949 |
| 466 | P | 0.4967 | 0.5846 | 0 | 0.0879 |
| 467 | D | 0.4245 | 0.5253 | 0 | 0.1008 |
| 468 | H | 0.4967 | 0.5253 | 0 | 0.0286 |
| 469 | H | 0.4078 | 0.4458 | 0 | 0.038 |
| 470 | I | 0.4918 | 0.5296 | 0 | 0.0378 |
| 471 | G | 0.4749 | 0.5126 | 0 | 0.0377 |
| 472 | E | 0.4749 | 0.5173 | 0 | 0.0424 |
| 473 | K | 0.5043 | 0.5412 | 0 | 0.0369 |
| 474 | R | 0.4078 | 0.4513 | 0 | 0.0435 |
| 475 | T | 0.4879 | 0.5331 | 0 | 0.0452 |
| 476 | S | 0.4037 | 0.4458 | 0 | 0.0421 |
| 477 | L | 0.3668 | 0.4245 | 0 | 0.0577 |
| 478 | D | 0.3578 | 0.4149 | 0 | 0.0571 |
| 479 | L | 0.363 | 0.4149 | 0 | 0.0519 |
| 480 | N | 0.3578 | 0.4149 | 0 | 0.0571 |
| 481 | M | 0.3456 | 0.4078 | 0 | 0.0622 |
| 482 | A | 0.3399 | 0.4037 | 0 | 0.0638 |
| 483 | P | 0.3311 | 0.3885 | 0 | 0.0574 |
| 484 | F | 0.2558 | 0.3184 | 0 | 0.0626 |
| 485 | Q | 0.2602 | 0.3225 | 0 | 0.0623 |
| 486 | I | 0.2385 | 0.3005 | 0 | 0.062 |
| 487 | H | 0.1667 | 0.2255 | 0 | 0.0588 |
| 488 | E | 0.1456 | 0.2913 | 0 | 0.1457 |
| 489 | K | 0.1495 | 0.2963 | 0 | 0.1468 |
| 490 | H | 0.1998 | 0.3578 | 0 | 0.158 |
| 491 | L | 0.1456 | 0.2865 | 0 | 0.1409 |
| 492 | S | 0.1667 | 0.3096 | 0 | 0.1429 |
| 493 | R | 0.1205 | 0.2531 | 0 | 0.1326 |
| 494 | L | 0.1018 | 0.2209 | 0 | 0.1191 |
| 495 | R | 0.1667 | 0.3146 | 0 | 0.1479 |
| 496 | A | 0.1456 | 0.2865 | 0 | 0.1409 |
| 497 | L | 0.1456 | 0.2913 | 0 | 0.1457 |
| 498 | S | 0.0909 | 0.1998 | 0 | 0.1089 |
| 499 | K | 0.0858 | 0.1958 | 0 | 0.11 |
| 500 | T | 0.0789 | 0.1844 | 0 | 0.1055 |
| 501 | V | 0.0455 | 0.1805 | 0 | 0.135 |
| 502 | E | 0.0744 | 0.2531 | 0 | 0.1787 |
| 503 | L | 0.049 | 0.1878 | 0 | 0.1388 |
| 504 | G | 0.0464 | 0.1844 | 0 | 0.138 |
| 505 | K | 0.0744 | 0.2602 | 0 | 0.1858 |
| 506 | R | 0.0455 | 0.1878 | 0 | 0.1423 |
| 507 | Y | 0.049 | 0.1958 | 0 | 0.1468 |
| 508 | F | 0.0744 | 0.2602 | 0 | 0.1858 |
| 509 | K | 0.1266 | 0.2602 | 0 | 0.1336 |
| 510 | R | 0.124 | 0.2531 | 0 | 0.1291 |
| 511 | S | 0.124 | 0.2531 | 0 | 0.1291 |
| 512 | S | 0.124 | 0.2558 | 0 | 0.1318 |
| 513 | L | 0.124 | 0.2558 | 0 | 0.1318 |
| 514 | D | 0.1878 | 0.3399 | 0 | 0.1521 |
| 515 | H | 0.138 | 0.2786 | 0 | 0.1406 |
| 516 | F | 0.1266 | 0.2602 | 0 | 0.1336 |
| 517 | M | 0.138 | 0.2748 | 0 | 0.1368 |
| 518 | D | 0.1495 | 0.2865 | 0 | 0.137 |
| 519 | T | 0.2255 | 0.3762 | 0 | 0.1507 |
| 520 | E | 0.2333 | 0.3806 | 0 | 0.1473 |
| 521 | D | 0.2432 | 0.3885 | 0 | 0.1453 |
| 522 | L | 0.3311 | 0.3847 | 0 | 0.0536 |
| 523 | N | 0.3668 | 0.4203 | 0 | 0.0535 |
| 524 | H | 0.442 | 0.4918 | 0 | 0.0498 |
| 525 | L | 0.4379 | 0.4825 | 0 | 0.0446 |
| 526 | A | 0.4245 | 0.4651 | 0 | 0.0406 |
| 527 | S | 0.4556 | 0.4918 | 0 | 0.0362 |
| 528 | V | 0.4825 | 0.5173 | 0 | 0.0348 |
| 529 | E | 0.4703 | 0.5084 | 0 | 0.0381 |
| 530 | E | 0.46 | 0.4967 | 0 | 0.0367 |
| 531 | D | 0.4749 | 0.5084 | 0 | 0.0335 |
| 532 | T | 0.4703 | 0.5043 | 0 | 0.034 |
| 533 | P | 0.4879 | 0.5229 | 0 | 0.035 |
| 534 | E | 0.4825 | 0.5126 | 0 | 0.0301 |
| 535 | K | 0.4918 | 0.5173 | 0 | 0.0255 |
| 536 | R | 0.5126 | 0.5374 | 0 | 0.0248 |
| 537 | L | 0.5412 | 0.5711 | 0 | 0.0299 |
| 538 | Q | 0.5412 | 0.5711 | 0 | 0.0299 |
| 539 | K | 0.5941 | 0.6219 | 0 | 0.0278 |
| 540 | K | 0.5583 | 0.59 | 0 | 0.0317 |
| 541 | Q | 0.5549 | 0.5846 | 0 | 0.0297 |
| 542 | R | 0.5473 | 0.5762 | 0 | 0.0289 |
| 543 | Y | 0.5514 | 0.5802 | 0 | 0.0288 |
| 544 | M | 0.4703 | 0.5043 | 0 | 0.034 |
| 545 | E | 0.4651 | 0.4967 | 0 | 0.0316 |
| 546 | L | 0.4749 | 0.5043 | 0 | 0.0294 |
| 547 | Q | 0.4879 | 0.5126 | 0 | 0.0247 |
| 548 | E | 0.5374 | 0.5667 | 0 | 0.0293 |
| 549 | T | 0.5229 | 0.5473 | 0 | 0.0244 |
| 550 | L | 0.5229 | 0.5473 | 0 | 0.0244 |
| 551 | M | 0.4825 | 0.5583 | 0 | 0.0758 |
| 552 | K | 0.4825 | 0.5583 | 0 | 0.0758 |
| 553 | T | 0.4918 | 0.5667 | 0 | 0.0749 |
| 554 | F | 0.5374 | 0.6124 | 0 | 0.075 |
| 555 | S | 0.5473 | 0.6174 | 0 | 0.0701 |
| 556 | E | 0.5374 | 0.6079 | 0 | 0.0705 |
| 557 | D | 0.5992 | 0.665 | 0 | 0.0658 |
| 558 | K | 0.5846 | 0.6516 | 0 | 0.067 |
| 559 | E | 0.6124 | 0.6756 | 0 | 0.0632 |
| 560 | E | 0.6124 | 0.6756 | 0 | 0.0632 |
| 561 | S | 0.6412 | 0.7034 | 0 | 0.0622 |
| 562 | G | 0.6334 | 0.6944 | 0 | 0.061 |
| 563 | K | 0.6035 | 0.6681 | 0 | 0.0646 |
| 564 | S | 0.6174 | 0.6756 | 0 | 0.0582 |
| 565 | S | 0.6557 | 0.7147 | 0 | 0.059 |
| 566 | T | 0.6604 | 0.7232 | 0 | 0.0628 |
| 567 | P | 0.6681 | 0.7317 | 0 | 0.0636 |
| 568 | K | 0.6715 | 0.7317 | 0 | 0.0602 |
| 569 | P | 0.6334 | 0.6944 | 0 | 0.061 |
| 570 | T | 0.6412 | 0.7034 | 0 | 0.0622 |
| 571 | S | 0.6604 | 0.7275 | 0 | 0.0671 |
| 572 | A | 0.7147 | 0.7342 | 0 | 0.0195 |
| 573 | V | 0.7192 | 0.7342 | 0 | 0.015 |
| 574 | R | 0.6827 | 0.7034 | 0 | 0.0207 |
| 575 | S | 0.6906 | 0.7111 | 0 | 0.0205 |
| 576 | N | 0.6516 | 0.6756 | 0 | 0.024 |
| 577 | R | 0.6474 | 0.6681 | 0 | 0.0207 |
| 578 | K | 0.6334 | 0.6557 | 0 | 0.0223 |
| 579 | L | 0.6474 | 0.6681 | 0 | 0.0207 |
| 580 | S | 0.6124 | 0.6334 | 0 | 0.021 |
| 581 | H | 0.5549 | 0.5762 | 0 | 0.0213 |
| 582 | R | 0.5229 | 0.5412 | 0 | 0.0183 |
| 583 | R | 0.5412 | 0.5623 | 0 | 0.0211 |
| 584 | L | 0.59 | 0.6079 | 0 | 0.0179 |
| 585 | K | 0.6124 | 0.6293 | 0 | 0.0169 |
| 586 | V | 0.5583 | 0.5762 | 0 | 0.0179 |
| 587 | D | 0.5583 | 0.5762 | 0 | 0.0179 |
| 588 | K | 0.5667 | 0.5846 | 0 | 0.0179 |
| 589 | R | 0.5711 | 0.59 | 0 | 0.0189 |
| 590 | D | 0.59 | 0.6035 | 0 | 0.0135 |
| 591 | F | 0.6293 | 0.6412 | 0 | 0.0119 |
| 592 | L | 0.6604 | 0.6681 | 0 | 0.0077 |
| 593 | K | 0.687 | 0.6944 | 0 | 0.0074 |
| 594 | R | 0.7147 | 0.7192 | 0 | 0.0045 |
| 595 | P | 0.7817 | 0.7869 | 0 | 0.0052 |
| 596 | Y | 0.8118 | 0.8158 | 0 | 0.004 |
| 597 | G | 0.8746 | 0.8746 | 0 | 0 |
| 598 | N | 0.9119 | 0.9119 | 0 | 0 |
| 599 | G | 0.9257 | 0.9257 | 0 | 0 |
| 600 | D | 0.9392 | 0.9392 | 0 | 0 |
| >AtNPR3 | | | | | |
| (AT5G45110) | | | | | |
| 1 | M | 0.9009 | 0.9009 | 0 | 0 |
| 2 | A | 0.8626 | 0.8626 | 0 | 0 |
| 3 | T | 0.7573 | 0.7573 | 0 | 0 |
| 4 | L | 0.7192 | 0.7192 | 0 | 0 |
| 5 | T | 0.6715 | 0.6715 | 0 | 0 |
| 6 | E | 0.6255 | 0.6255 | 0 | 0 |
| 7 | P | 0.5846 | 0.5846 | 0 | 0 |
| 8 | S | 0.4703 | 0.4703 | 0 | 0 |
| 9 | S | 0.4149 | 0.4149 | 0 | 0 |
| 10 | S | 0.3005 | 0.3005 | 0 | 0 |
| 11 | L | 0.2657 | 0.2657 | 0 | 0 |
| 12 | S | 0.2748 | 0.2748 | 0 | 0 |
| 13 | F | 0.2385 | 0.2385 | 0 | 0 |
| 14 | T | 0.2483 | 0.2483 | 0 | 0 |
| 15 | S | 0.2963 | 0.2963 | 0 | 0 |
| 16 | S | 0.3053 | 0.3053 | 0 | 0 |
| 17 | H | 0.2865 | 0.2865 | 0 | 0 |
| 18 | F | 0.1844 | 0.1844 | 0 | 0 |
| 19 | S | 0.1878 | 0.1878 | 0 | 0 |
| 20 | Y | 0.1921 | 0.1921 | 0 | 0 |
| 21 | G | 0.1958 | 0.1958 | 0 | 0 |
| 22 | S | 0.2292 | 0.2292 | 0 | 0 |
| 23 | I | 0.2122 | 0.2167 | 0 | 0.0045 |
| 24 | G | 0.282 | 0.282 | 0 | 0 |
| 25 | S | 0.2865 | 0.2865 | 0 | 0 |
| 26 | N | 0.3263 | 0.3311 | 0 | 0.0048 |
| 27 | H | 0.3578 | 0.363 | 0 | 0.0052 |
| 28 | F | 0.3053 | 0.3096 | 0 | 0.0043 |
| 29 | S | 0.3399 | 0.3491 | 0 | 0.0092 |
| 30 | S | 0.3456 | 0.3491 | 0 | 0.0035 |
| 31 | S | 0.3456 | 0.3535 | 0 | 0.0079 |
| 32 | S | 0.3311 | 0.3399 | 0 | 0.0088 |
| 33 | A | 0.3311 | 0.3456 | 0 | 0.0145 |
| 34 | S | 0.3263 | 0.3399 | 0 | 0.0136 |
| 35 | N | 0.3184 | 0.3311 | 0 | 0.0127 |
| 36 | P | 0.3263 | 0.3359 | 0 | 0.0096 |
| 37 | E | 0.3225 | 0.3359 | 0 | 0.0134 |
| 38 | V | 0.2483 | 0.2602 | 0 | 0.0119 |
| 39 | V | 0.3491 | 0.3668 | 0 | 0.0177 |
| 40 | S | 0.3762 | 0.3939 | 0 | 0.0177 |
| 41 | L | 0.3184 | 0.3399 | 0 | 0.0215 |
| 42 | T | 0.2602 | 0.2865 | 0 | 0.0263 |
| 43 | K | 0.2657 | 0.2913 | 0 | 0.0256 |
| 44 | L | 0.2913 | 0.3225 | 0 | 0.0312 |
| 45 | S | 0.3005 | 0.3263 | 0 | 0.0258 |
| 46 | S | 0.3146 | 0.3399 | 0 | 0.0253 |
| 47 | N | 0.208 | 0.3096 | 0 | 0.1016 |
| 48 | L | 0.1998 | 0.3005 | 0 | 0.1007 |
| 49 | E | 0.1766 | 0.2786 | 0 | 0.102 |
| 50 | Q | 0.2209 | 0.3184 | 0 | 0.0975 |
| 51 | L | 0.2385 | 0.3399 | 0 | 0.1014 |
| 52 | L | 0.27 | 0.3762 | 0 | 0.1062 |
| 53 | S | 0.2913 | 0.3939 | 0 | 0.1026 |
| 54 | N | 0.2122 | 0.3184 | 0 | 0.1062 |
| 55 | S | 0.2041 | 0.3005 | 0 | 0.0964 |
| 56 | D | 0.1495 | 0.2385 | 0 | 0.089 |
| 57 | S | 0.1566 | 0.2531 | 0 | 0.0965 |
| 58 | D | 0.1566 | 0.2531 | 0 | 0.0965 |
| 59 | Y | 0.1566 | 0.2558 | 0 | 0.0992 |
| 60 | S | 0.1698 | 0.2748 | 0 | 0.105 |
| 61 | D | 0.1088 | 0.1921 | 0 | 0.0833 |
| 62 | A | 0.1532 | 0.2531 | 0 | 0.0999 |
| 63 | E | 0.1532 | 0.2531 | 0 | 0.0999 |
| 64 | I | 0.1532 | 0.2602 | 0 | 0.107 |
| 65 | I | 0.1495 | 0.2558 | 0 | 0.1063 |
| 66 | V | 0.0965 | 0.2558 | 0 | 0.1593 |
| 67 | D | 0.0587 | 0.1805 | 0 | 0.1218 |
| 68 | G | 0.0643 | 0.1349 | 0 | 0.0706 |
| 69 | V | 0.049 | 0.1088 | 0 | 0.0598 |
| 70 | P | 0.0621 | 0.138 | 0 | 0.0759 |
| 71 | V | 0.0621 | 0.138 | 0 | 0.0759 |
| 72 | G | 0.0567 | 0.1292 | 0 | 0.0725 |
| 73 | V | 0.0723 | 0.1532 | 0 | 0.0809 |
| 74 | H | 0.0395 | 0.0858 | 0 | 0.0463 |
| 75 | R | 0.0405 | 0.0909 | 0 | 0.0504 |
| 76 | S | 0.0677 | 0.1495 | 0 | 0.0818 |
| 77 | I | 0.1088 | 0.208 | 0 | 0.0992 |
| 78 | L | 0.0643 | 0.1416 | 0 | 0.0773 |
| 79 | A | 0.0376 | 0.0832 | 0 | 0.0456 |
| 80 | A | 0.0554 | 0.124 | 0 | 0.0686 |
| 81 | R | 0.0464 | 0.1018 | 0 | 0.0554 |
| 82 | S | 0.0723 | 0.1495 | 0 | 0.0772 |
| 83 | K | 0.0744 | 0.1495 | 0 | 0.0751 |
| 84 | F | 0.115 | 0.208 | 0 | 0.093 |
| 85 | F | 0.0723 | 0.138 | 0 | 0.0657 |
| 86 | Q | 0.0723 | 0.138 | 0 | 0.0657 |
| 87 | D | 0.1205 | 0.1416 | 0 | 0.0211 |
| 88 | L | 0.1635 | 0.1878 | 0 | 0.0243 |
| 89 | F | 0.2209 | 0.2483 | 0 | 0.0274 |
| 90 | K | 0.2531 | 0.2748 | 0 | 0.0217 |
| 91 | K | 0.3146 | 0.3359 | 0 | 0.0213 |
| 92 | E | 0.3146 | 0.3359 | 0 | 0.0213 |
| 93 | K | 0.2255 | 0.2483 | 0 | 0.0228 |
| 94 | K | 0.2385 | 0.2602 | 0 | 0.0217 |
| 95 | I | 0.2432 | 0.2602 | 0 | 0.017 |
| 96 | S | 0.3359 | 0.3491 | 0 | 0.0132 |
| 97 | K | 0.3311 | 0.3456 | 0 | 0.0145 |
| 98 | T | 0.2786 | 0.2913 | 0 | 0.0127 |
| 99 | E | 0.2657 | 0.2748 | 0 | 0.0091 |
| 100 | K | 0.3847 | 0.3939 | 0 | 0.0092 |
| 101 | P | 0.3005 | 0.3096 | 0 | 0.0091 |
| 102 | K | 0.2963 | 0.3053 | 0 | 0.009 |
| 103 | Y | 0.2558 | 0.2602 | 0 | 0.0044 |
| 104 | Q | 0.1844 | 0.1878 | 0 | 0.0034 |
| 105 | L | 0.1566 | 0.1566 | 0 | 0 |
| 106 | R | 0.2209 | 0.2292 | 0 | 0.0083 |
| 107 | E | 0.2255 | 0.2333 | 0 | 0.0078 |
| 108 | M | 0.1878 | 0.1998 | 0 | 0.012 |
| 109 | L | 0.1205 | 0.1292 | 0 | 0.0087 |
| 110 | P | 0.0744 | 0.0813 | 0 | 0.0069 |
| 111 | Y | 0.0464 | 0.0502 | 0 | 0.0038 |
| 112 | G | 0.0194 | 0.0231 | 0 | 0.0037 |
| 113 | A | 0.0109 | 0.0128 | 0 | 0.0019 |
| 114 | V | 0.0218 | 0.0259 | 0 | 0.0041 |
| 115 | A | 0.0102 | 0.0128 | 0 | 0.0026 |
| 116 | H | 0.0109 | 0.0131 | 0 | 0.0022 |
| 117 | E | 0.0066 | 0.0078 | 0 | 0.0012 |
| 118 | A | 0.0061 | 0.0078 | 0 | 0.0017 |
| 119 | F | 0.0078 | 0.0099 | 0 | 0.0021 |
| 120 | L | 0.0131 | 0.02 | 0 | 0.0069 |
| 121 | Y | 0.0062 | 0.0086 | 0 | 0.0024 |
| 122 | F | 0.0094 | 0.015 | 0 | 0.0056 |
| 123 | L | 0.0109 | 0.0179 | 0 | 0.007 |
| 124 | S | 0.0067 | 0.0099 | 0 | 0.0032 |
| 125 | Y | 0.0118 | 0.0218 | 0 | 0.01 |
| 126 | I | 0.0078 | 0.0131 | 0 | 0.0053 |
| 127 | Y | 0.0083 | 0.015 | 0 | 0.0067 |
| 128 | T | 0.0044 | 0.0083 | 0 | 0.0039 |
| 129 | G | 0.0062 | 0.0099 | 0 | 0.0037 |
| 130 | R | 0.0102 | 0.02 | 0 | 0.0098 |
| 131 | L | 0.0074 | 0.0316 | 0 | 0.0242 |
| 132 | K | 0.007 | 0.0308 | 0 | 0.0238 |
| 133 | P | 0.0137 | 0.0607 | 0 | 0.047 |
| 134 | F | 0.0297 | 0.1117 | 0 | 0.082 |
| 135 | P | 0.016 | 0.0701 | 0 | 0.0541 |
| 136 | L | 0.0109 | 0.1117 | 0 | 0.1008 |
| 137 | E | 0.0194 | 0.1635 | 0 | 0.1441 |
| 138 | V | 0.0304 | 0.2292 | 0 | 0.1988 |
| 139 | S | 0.035 | 0.2531 | 0 | 0.2181 |
| 140 | T | 0.0173 | 0.2483 | 0 | 0.231 |
| 141 | S | 0.0084 | 0.2483 | 0 | 0.2399 |
| 142 | V | 0.015 | 0.3263 | 0 | 0.3113 |
| 143 | D | 0.02 | 0.3578 | 0 | 0.3378 |
| 144 | P | 0.0128 | 0.3053 | 0 | 0.2925 |
| 145 | V | 0.0179 | 0.3456 | 0 | 0.3277 |
| 146 | S | 0.0173 | 0.3399 | 0 | 0.3226 |
| 147 | S | 0.0167 | 0.3359 | 0 | 0.3192 |
| 148 | H | 0.009 | 0.2602 | 0 | 0.2512 |
| 149 | D | 0.0097 | 0.27 | 0 | 0.2603 |
| 150 | S | 0.0097 | 0.2865 | 0 | 0.2768 |
| 151 | S | 0.0062 | 0.2122 | 0 | 0.206 |
| 152 | R | 0.0121 | 0.1958 | 0 | 0.1837 |
| 153 | P | 0.0128 | 0.208 | 0 | 0.1952 |
| 154 | A | 0.0087 | 0.1698 | 0 | 0.1611 |
| 155 | I | 0.0081 | 0.1495 | 0 | 0.1414 |
| 156 | D | 0.0141 | 0.2167 | 0 | 0.2026 |
| 157 | F | 0.0245 | 0.1635 | 0 | 0.139 |
| 158 | V | 0.0124 | 0.1088 | 0 | 0.0964 |
| 159 | V | 0.0141 | 0.115 | 0 | 0.1009 |
| 160 | Q | 0.0078 | 0.0643 | 0 | 0.0565 |
| 161 | L | 0.0182 | 0.0789 | 0 | 0.0607 |
| 162 | M | 0.0414 | 0.0858 | 0 | 0.0444 |
| 163 | Y | 0.0226 | 0.049 | 0 | 0.0264 |
| 164 | A | 0.0097 | 0.0252 | 0 | 0.0155 |
| 165 | S | 0.0131 | 0.0297 | 0 | 0.0166 |
| 166 | S | 0.0252 | 0.049 | 0 | 0.0238 |
| 167 | V | 0.0084 | 0.0182 | 0 | 0.0098 |
| 168 | L | 0.0179 | 0.0376 | 0 | 0.0197 |
| 169 | Q | 0.0279 | 0.0514 | 0 | 0.0235 |
| 170 | V | 0.0425 | 0.0789 | 0 | 0.0364 |
| 171 | P | 0.0218 | 0.0376 | 0 | 0.0158 |
| 172 | E | 0.0218 | 0.0607 | 0 | 0.0389 |
| 173 | L | 0.0279 | 0.0744 | 0 | 0.0465 |
| 174 | V | 0.0194 | 0.0526 | 0 | 0.0332 |
| 175 | S | 0.0137 | 0.0387 | 0 | 0.025 |
| 176 | S | 0.015 | 0.0425 | 0 | 0.0275 |
| 177 | F | 0.0144 | 0.0441 | 0 | 0.0297 |
| 178 | Q | 0.0245 | 0.0643 | 0 | 0.0398 |
| 179 | R | 0.0218 | 0.0587 | 0 | 0.0369 |
| 180 | R | 0.0121 | 0.0336 | 0 | 0.0215 |
| 181 | L | 0.0231 | 0.0554 | 0 | 0.0323 |
| 182 | S | 0.0194 | 0.0502 | 0 | 0.0308 |
| 183 | N | 0.0094 | 0.0279 | 0 | 0.0185 |
| 184 | F | 0.0094 | 0.0286 | 0 | 0.0192 |
| 185 | V | 0.0231 | 0.0567 | 0 | 0.0336 |
| 186 | E | 0.0109 | 0.0308 | 0 | 0.0199 |
| 187 | K | 0.0061 | 0.016 | 0 | 0.0099 |
| 188 | T | 0.0099 | 0.0308 | 0 | 0.0209 |
| 189 | L | 0.0066 | 0.0173 | 0 | 0.0107 |
| 190 | V | 0.0052 | 0.0141 | 0 | 0.0089 |
| 191 | E | 0.0015 | 0.007 | 0 | 0.0055 |
| 192 | N | 0.0044 | 0.0144 | 0 | 0.01 |
| 193 | V | 0.0033 | 0.0144 | 0 | 0.0111 |
| 194 | L | 0.0029 | 0.0137 | 0 | 0.0108 |
| 195 | P | 0.0033 | 0.0157 | 0 | 0.0124 |
| 196 | I | 0.0066 | 0.0297 | 0 | 0.0231 |
| 197 | L | 0.0055 | 0.0286 | 0 | 0.0231 |
| 198 | M | 0.0028 | 0.015 | 0 | 0.0122 |
| 199 | V | 0.0009 | 0.0082 | 0 | 0.0073 |
| 200 | A | 0.0033 | 0.0179 | 0 | 0.0146 |
| 201 | F | 0.0062 | 0.0316 | 0 | 0.0254 |
| 202 | N | 0.0026 | 0.0304 | 0 | 0.0278 |
| 203 | S | 0.0008 | 0.0137 | 0 | 0.0129 |
| 204 | K | 0.0026 | 0.0279 | 0 | 0.0253 |
| 205 | L | 0.0049 | 0.0478 | 0 | 0.0429 |
| 206 | T | 0.0015 | 0.0245 | 0 | 0.023 |
| 207 | Q | 0.0031 | 0.0363 | 0 | 0.0332 |
| 208 | L | 0.0067 | 0.0607 | 0 | 0.054 |
| 209 | L | 0.0081 | 0.0771 | 0 | 0.069 |
| 210 | D | 0.015 | 0.1292 | 0 | 0.1142 |
| 211 | Q | 0.0099 | 0.0965 | 0 | 0.0866 |
| 212 | S | 0.0137 | 0.1178 | 0 | 0.1041 |
| 213 | I | 0.0131 | 0.1117 | 0 | 0.0986 |
| 214 | E | 0.0194 | 0.0677 | 0 | 0.0483 |
| 215 | R | 0.0094 | 0.0723 | 0 | 0.0629 |
| 216 | V | 0.0118 | 0.0813 | 0 | 0.0695 |
| 217 | A | 0.0128 | 0.0858 | 0 | 0.073 |
| 218 | R | 0.0124 | 0.0789 | 0 | 0.0665 |
| 219 | S | 0.0268 | 0.1349 | 0 | 0.1081 |
| 220 | D | 0.0304 | 0.1495 | 0 | 0.1191 |
| 221 | L | 0.0363 | 0.1698 | 0 | 0.1335 |
| 222 | Y | 0.0464 | 0.1958 | 0 | 0.1494 |
| 223 | R | 0.0909 | 0.1998 | 0 | 0.1089 |
| 224 | F | 0.1117 | 0.2385 | 0 | 0.1268 |
| 225 | S | 0.0935 | 0.2122 | 0 | 0.1187 |
| 226 | I | 0.1018 | 0.2209 | 0 | 0.1191 |
| 227 | E | 0.1495 | 0.2913 | 0 | 0.1418 |
| 228 | K | 0.124 | 0.2483 | 0 | 0.1243 |
| 229 | E | 0.124 | 0.2483 | 0 | 0.1243 |
| 230 | V | 0.1266 | 0.2558 | 0 | 0.1292 |
| 231 | P | 0.0789 | 0.1805 | 0 | 0.1016 |
| 232 | P | 0.1292 | 0.2558 | 0 | 0.1266 |
| 233 | E | 0.138 | 0.2657 | 0 | 0.1277 |
| 234 | V | 0.0991 | 0.1998 | 0 | 0.1007 |
| 235 | A | 0.1602 | 0.282 | 0 | 0.1218 |
| 236 | E | 0.282 | 0.3146 | 0 | 0.0326 |
| 237 | K | 0.363 | 0.3992 | 0 | 0.0362 |
| 238 | I | 0.3668 | 0.3992 | 0 | 0.0324 |
| 239 | K | 0.3806 | 0.4116 | 0 | 0.031 |
| 240 | Q | 0.3806 | 0.4078 | 0 | 0.0272 |
| 241 | L | 0.4458 | 0.4781 | 0 | 0.0323 |
| 242 | R | 0.4149 | 0.442 | 0 | 0.0271 |
| 243 | L | 0.4149 | 0.442 | 0 | 0.0271 |
| 244 | I | 0.4037 | 0.4245 | 0 | 0.0208 |
| 245 | S | 0.3806 | 0.4037 | 0 | 0.0231 |
| 246 | P | 0.4116 | 0.4282 | 0 | 0.0166 |
| 247 | Q | 0.4149 | 0.4333 | 0 | 0.0184 |
| 248 | D | 0.4149 | 0.4282 | 0 | 0.0133 |
| 249 | E | 0.4078 | 0.4245 | 0 | 0.0167 |
| 250 | E | 0.3399 | 0.3578 | 0 | 0.0179 |
| 251 | T | 0.3456 | 0.3535 | 0 | 0.0079 |
| 252 | S | 0.4116 | 0.4149 | 0 | 0.0033 |
| 253 | P | 0.3263 | 0.3359 | 0 | 0.0096 |
| 254 | K | 0.4078 | 0.4116 | 0 | 0.0038 |
| 255 | I | 0.46 | 0.4651 | 0 | 0.0051 |
| 256 | S | 0.3717 | 0.3806 | 0 | 0.0089 |
| 257 | E | 0.2657 | 0.27 | 0 | 0.0043 |
| 258 | K | 0.2432 | 0.2483 | 0 | 0.0051 |
| 259 | L | 0.2122 | 0.2122 | 0 | 0 |
| 260 | L | 0.1456 | 0.1456 | 0 | 0 |
| 261 | E | 0.1495 | 0.1495 | 0 | 0 |
| 262 | R | 0.1532 | 0.1532 | 0 | 0 |
| 263 | I | 0.1532 | 0.1532 | 0 | 0 |
| 264 | G | 0.1292 | 0.1292 | 0 | 0 |
| 265 | K | 0.0935 | 0.0935 | 0 | 0 |
| 266 | I | 0.1602 | 0.1602 | 0 | 0 |
| 267 | L | 0.0935 | 0.0935 | 0 | 0 |
| 268 | K | 0.0567 | 0.0567 | 0 | 0 |
| 269 | A | 0.0567 | 0.0567 | 0 | 0 |
| 270 | L | 0.0542 | 0.0542 | 0 | 0 |
| 271 | D | 0.0526 | 0.0526 | 0 | 0 |
| 272 | S | 0.0286 | 0.0286 | 0 | 0 |
| 273 | D | 0.0279 | 0.0279 | 0 | 0 |
| 274 | D | 0.0542 | 0.0542 | 0 | 0 |
| 275 | V | 0.0514 | 0.0514 | 0 | 0 |
| 276 | E | 0.0587 | 0.0587 | 0 | 0 |
| 277 | L | 0.0514 | 0.0514 | 0 | 0 |
| 278 | V | 0.0789 | 0.0789 | 0 | 0 |
| 279 | K | 0.0478 | 0.0478 | 0 | 0 |
| 280 | L | 0.0567 | 0.0567 | 0 | 0 |
| 281 | L | 0.0935 | 0.0935 | 0 | 0 |
| 282 | L | 0.0701 | 0.0701 | 0 | 0 |
| 283 | T | 0.0701 | 0.0701 | 0 | 0 |
| 284 | E | 0.0677 | 0.0677 | 0 | 0 |
| 285 | S | 0.0363 | 0.0363 | 0 | 0 |
| 286 | D | 0.0542 | 0.0542 | 0 | 0 |
| 287 | I | 0.0316 | 0.0316 | 0 | 0 |
| 288 | T | 0.0554 | 0.0554 | 0 | 0 |
| 289 | L | 0.0567 | 0.0567 | 0 | 0 |
| 290 | D | 0.0414 | 0.0414 | 0 | 0 |
| 291 | Q | 0.0455 | 0.0455 | 0 | 0 |
| 292 | A | 0.0744 | 0.0744 | 0 | 0 |
| 293 | N | 0.1322 | 0.1322 | 0 | 0 |
| 294 | G | 0.1635 | 0.1635 | 0 | 0 |
| 295 | L | 0.1456 | 0.1456 | 0 | 0 |
| 296 | H | 0.1018 | 0.1018 | 0 | 0 |
| 297 | Y | 0.0643 | 0.0643 | 0 | 0 |
| 298 | S | 0.1018 | 0.1018 | 0 | 0 |
| 299 | V | 0.115 | 0.115 | 0 | 0 |
| 300 | V | 0.1205 | 0.1205 | 0 | 0 |
| 301 | Y | 0.0701 | 0.0701 | 0 | 0 |
| 302 | S | 0.0542 | 0.0542 | 0 | 0 |
| 303 | D | 0.0425 | 0.0425 | 0 | 0 |
| 304 | P | 0.0441 | 0.0441 | 0 | 0 |
| 305 | K | 0.0327 | 0.0327 | 0 | 0 |
| 306 | V | 0.0567 | 0.0567 | 0 | 0 |
| 307 | V | 0.0621 | 0.0621 | 0 | 0 |
| 308 | A | 0.0621 | 0.0621 | 0 | 0 |
| 309 | E | 0.0621 | 0.0621 | 0 | 0 |
| 310 | I | 0.0514 | 0.0514 | 0 | 0 |
| 311 | L | 0.0744 | 0.0744 | 0 | 0 |
| 312 | A | 0.1178 | 0.1178 | 0 | 0 |
| 313 | L | 0.1178 | 0.1178 | 0 | 0 |
| 314 | D | 0.1088 | 0.1088 | 0 | 0 |
| 315 | M | 0.0909 | 0.0909 | 0 | 0 |
| 316 | G | 0.0554 | 0.0554 | 0 | 0 |
| 317 | D | 0.0858 | 0.0858 | 0 | 0 |
| 318 | V | 0.0832 | 0.0832 | 0 | 0 |
| 319 | N | 0.0621 | 0.0621 | 0 | 0 |
| 320 | Y | 0.0567 | 0.0567 | 0 | 0 |
| 321 | R | 0.0542 | 0.0542 | 0 | 0 |
| 322 | N | 0.0744 | 0.0744 | 0 | 0 |
| 323 | S | 0.0744 | 0.0744 | 0 | 0 |
| 324 | R | 0.0991 | 0.0991 | 0 | 0 |
| 325 | G | 0.0909 | 0.0909 | 0 | 0 |
| 326 | Y | 0.1205 | 0.1205 | 0 | 0 |
| 327 | T | 0.1322 | 0.1322 | 0 | 0 |
| 328 | V | 0.1495 | 0.1495 | 0 | 0 |
| 329 | L | 0.208 | 0.208 | 0 | 0 |
| 330 | H | 0.1416 | 0.1416 | 0 | 0 |
| 331 | F | 0.1566 | 0.1566 | 0 | 0 |
| 332 | A | 0.106 | 0.106 | 0 | 0 |
| 333 | A | 0.106 | 0.106 | 0 | 0 |
| 334 | M | 0.066 | 0.066 | 0 | 0 |
| 335 | R | 0.0414 | 0.0414 | 0 | 0 |
| 336 | R | 0.0425 | 0.0425 | 0 | 0 |
| 337 | E | 0.0744 | 0.0744 | 0 | 0 |
| 338 | P | 0.0771 | 0.0771 | 0 | 0 |
| 339 | S | 0.0935 | 0.0935 | 0 | 0 |
| 340 | I | 0.1495 | 0.1495 | 0 | 0 |
| 341 | I | 0.124 | 0.124 | 0 | 0 |
| 342 | I | 0.1998 | 0.1998 | 0 | 0 |
| 343 | S | 0.2385 | 0.2385 | 0 | 0 |
| 344 | L | 0.1878 | 0.1878 | 0 | 0 |
| 345 | I | 0.2209 | 0.2209 | 0 | 0 |
| 346 | D | 0.2209 | 0.2209 | 0 | 0 |
| 347 | K | 0.2167 | 0.2167 | 0 | 0 |
| 348 | G | 0.208 | 0.208 | 0 | 0 |
| 349 | A | 0.1844 | 0.1844 | 0 | 0 |
| 350 | N | 0.1844 | 0.1844 | 0 | 0 |
| 351 | A | 0.2385 | 0.2385 | 0 | 0 |
| 352 | S | 0.2602 | 0.2602 | 0 | 0 |
| 353 | E | 0.3399 | 0.3399 | 0 | 0 |
| 354 | F | 0.2786 | 0.2786 | 0 | 0 |
| 355 | T | 0.2913 | 0.2963 | 0 | 0.005 |
| 356 | S | 0.3806 | 0.3806 | 0 | 0 |
| 357 | D | 0.3847 | 0.3847 | 0 | 0 |
| 358 | G | 0.3146 | 0.3225 | 0 | 0.0079 |
| 359 | R | 0.3096 | 0.3146 | 0 | 0.005 |
| 360 | S | 0.3399 | 0.3491 | 0 | 0.0092 |
| 361 | A | 0.3762 | 0.3847 | 0 | 0.0085 |
| 362 | V | 0.4078 | 0.4149 | 0 | 0.0071 |
| 363 | N | 0.4037 | 0.4149 | 0 | 0.0112 |
| 364 | I | 0.3359 | 0.3491 | 0 | 0.0132 |
| 365 | L | 0.4282 | 0.4513 | 0 | 0.0231 |
| 366 | R | 0.4333 | 0.4513 | 0 | 0.018 |
| 367 | R | 0.4333 | 0.4556 | 0 | 0.0223 |
| 368 | L | 0.4379 | 0.46 | 0 | 0.0221 |
| 369 | T | 0.4149 | 0.4379 | 0 | 0.023 |
| 370 | N | 0.4149 | 0.442 | 0 | 0.0271 |
| 371 | P | 0.4149 | 0.4458 | 0 | 0.0309 |
| 372 | K | 0.442 | 0.4703 | 0 | 0.0283 |
| 373 | D | 0.4879 | 0.5173 | 0 | 0.0294 |
| 374 | Y | 0.4918 | 0.5229 | 0 | 0.0311 |
| 375 | H | 0.5583 | 0.5846 | 0 | 0.0263 |
| 376 | T | 0.6219 | 0.6442 | 0 | 0.0223 |
| 377 | K | 0.59 | 0.6174 | 0 | 0.0274 |
| 378 | T | 0.5846 | 0.6174 | 0 | 0.0328 |
| 379 | A | 0.5802 | 0.6174 | 0 | 0.0372 |
| 380 | K | 0.5229 | 0.6219 | 0 | 0.099 |
| 381 | G | 0.4651 | 0.5623 | 0 | 0.0972 |
| 382 | R | 0.4282 | 0.5331 | 0 | 0.1049 |
| 383 | E | 0.3762 | 0.4781 | 0 | 0.1019 |
| 384 | S | 0.3311 | 0.4333 | 0 | 0.1022 |
| 385 | S | 0.3992 | 0.5043 | 0 | 0.1051 |
| 386 | K | 0.3992 | 0.5008 | 0 | 0.1016 |
| 387 | A | 0.4037 | 0.5008 | 0 | 0.0971 |
| 388 | R | 0.3311 | 0.4282 | 0 | 0.0971 |
| 389 | L | 0.3263 | 0.4282 | 0 | 0.1019 |
| 390 | S | 0.3535 | 0.4513 | 0 | 0.0978 |
| 391 | I | 0.3456 | 0.4458 | 0 | 0.1002 |
| 392 | D | 0.3717 | 0.4781 | 0 | 0.1064 |
| 393 | I | 0.3491 | 0.4556 | 0 | 0.1065 |
| 394 | L | 0.2963 | 0.3992 | 0 | 0.1029 |
| 395 | E | 0.2167 | 0.3263 | 0 | 0.1096 |
| 396 | R | 0.2209 | 0.3311 | 0 | 0.1102 |
| 397 | E | 0.2167 | 0.3311 | 0 | 0.1144 |
| 398 | I | 0.2748 | 0.3885 | 0 | 0.1137 |
| 399 | R | 0.2432 | 0.3578 | 0 | 0.1146 |
| 400 | K | 0.2333 | 0.4245 | 0 | 0.1912 |
| 401 | N | 0.3096 | 0.4245 | 0 | 0.1149 |
| 402 | P | 0.27 | 0.3847 | 0 | 0.1147 |
| 403 | M | 0.2786 | 0.3885 | 0 | 0.1099 |
| 404 | V | 0.2963 | 0.4078 | 0 | 0.1115 |
| 405 | L | 0.3762 | 0.4879 | 0 | 0.1117 |
| 406 | D | 0.3762 | 0.4879 | 0 | 0.1117 |
| 407 | T | 0.3806 | 0.4918 | 0 | 0.1112 |
| 408 | P | 0.2865 | 0.3992 | 0 | 0.1127 |
| 409 | M | 0.363 | 0.4781 | 0 | 0.1151 |
| 410 | S | 0.3311 | 0.442 | 0 | 0.1109 |
| 411 | S | 0.3263 | 0.4379 | 0 | 0.1116 |
| 412 | I | 0.2531 | 0.363 | 0 | 0.1099 |
| 413 | S | 0.1566 | 0.2531 | 0 | 0.0965 |
| 414 | M | 0.1088 | 0.1878 | 0 | 0.079 |
| 415 | P | 0.0965 | 0.1732 | 0 | 0.0767 |
| 416 | E | 0.1566 | 0.2531 | 0 | 0.0965 |
| 417 | D | 0.1495 | 0.2385 | 0 | 0.089 |
| 418 | L | 0.1416 | 0.2292 | 0 | 0.0876 |
| 419 | Q | 0.0789 | 0.1416 | 0 | 0.0627 |
| 420 | M | 0.0935 | 0.1667 | 0 | 0.0732 |
| 421 | R | 0.0935 | 0.1088 | 0 | 0.0153 |
| 422 | L | 0.0723 | 0.0884 | 0 | 0.0161 |
| 423 | L | 0.1349 | 0.1602 | 0 | 0.0253 |
| 424 | Y | 0.0832 | 0.1041 | 0 | 0.0209 |
| 425 | L | 0.0607 | 0.0771 | 0 | 0.0164 |
| 426 | E | 0.0297 | 0.0376 | 0 | 0.0079 |
| 427 | K | 0.0336 | 0.0414 | 0 | 0.0078 |
| 428 | R | 0.0316 | 0.0395 | 0 | 0.0079 |
| 429 | V | 0.0621 | 0.0771 | 0 | 0.015 |
| 430 | G | 0.0478 | 0.0554 | 0 | 0.0076 |
| 431 | L | 0.0554 | 0.066 | 0 | 0.0106 |
| 432 | A | 0.0376 | 0.0441 | 0 | 0.0065 |
| 433 | Q | 0.0502 | 0.0567 | 0 | 0.0065 |
| 434 | L | 0.066 | 0.0771 | 0 | 0.0111 |
| 435 | F | 0.1205 | 0.1322 | 0 | 0.0117 |
| 436 | F | 0.1178 | 0.1292 | 0 | 0.0114 |
| 437 | P | 0.106 | 0.1178 | 0 | 0.0118 |
| 438 | T | 0.1117 | 0.1205 | 0 | 0.0088 |
| 439 | E | 0.0744 | 0.0832 | 0 | 0.0088 |
| 440 | A | 0.1292 | 0.138 | 0 | 0.0088 |
| 441 | K | 0.1292 | 0.138 | 0 | 0.0088 |
| 442 | V | 0.1878 | 0.1958 | 0 | 0.008 |
| 443 | A | 0.2209 | 0.2292 | 0 | 0.0083 |
| 444 | M | 0.2385 | 0.2385 | 0 | 0 |
| 445 | D | 0.2385 | 0.2385 | 0 | 0 |
| 446 | I | 0.3225 | 0.3225 | 0 | 0 |
| 447 | G | 0.4116 | 0.4116 | 0 | 0 |
| 448 | N | 0.3263 | 0.3263 | 0 | 0 |
| 449 | V | 0.3263 | 0.3263 | 0 | 0 |
| 450 | E | 0.3399 | 0.3399 | 0 | 0 |
| 451 | G | 0.4078 | 0.4078 | 0 | 0 |
| 452 | T | 0.4203 | 0.4203 | 0 | 0 |
| 453 | S | 0.4967 | 0.4967 | 0 | 0 |
| 454 | E | 0.5331 | 0.5331 | 0 | 0 |
| 455 | F | 0.5084 | 0.5084 | 0 | 0 |
| 456 | T | 0.4967 | 0.4967 | 0 | 0 |
| 457 | G | 0.5846 | 0.5846 | 0 | 0 |
| 458 | L | 0.5846 | 0.5846 | 0 | 0 |
| 459 | S | 0.5253 | 0.5253 | 0 | 0 |
| 460 | P | 0.5941 | 0.5941 | 0 | 0 |
| 461 | P | 0.5846 | 0.5846 | 0 | 0 |
| 462 | S | 0.5296 | 0.5296 | 0 | 0 |
| 463 | S | 0.5412 | 0.5412 | 0 | 0 |
| 464 | G | 0.4879 | 0.4879 | 0 | 0 |
| 465 | L | 0.4703 | 0.4703 | 0 | 0 |
| 466 | T | 0.5549 | 0.5549 | 0 | 0 |
| 467 | G | 0.5549 | 0.5549 | 0 | 0 |
| 468 | N | 0.5802 | 0.5802 | 0 | 0 |
| 469 | L | 0.6374 | 0.6374 | 0 | 0 |
| 470 | S | 0.6079 | 0.6079 | 0 | 0 |
| 471 | Q | 0.59 | 0.59 | 0 | 0 |
| 472 | V | 0.5583 | 0.5583 | 0 | 0 |
| 473 | D | 0.5667 | 0.5667 | 0 | 0 |
| 474 | L | 0.5711 | 0.5711 | 0 | 0 |
| 475 | N | 0.5084 | 0.5084 | 0 | 0 |
| 476 | E | 0.5084 | 0.5084 | 0 | 0 |
| 477 | T | 0.5043 | 0.5043 | 0 | 0 |
| 478 | P | 0.5043 | 0.5043 | 0 | 0 |
| 479 | H | 0.4651 | 0.4651 | 0 | 0 |
| 480 | M | 0.4651 | 0.4651 | 0 | 0 |
| 481 | Q | 0.4333 | 0.4333 | 0 | 0 |
| 482 | T | 0.3578 | 0.3578 | 0 | 0 |
| 483 | Q | 0.3717 | 0.3717 | 0 | 0 |
| 484 | R | 0.3535 | 0.3535 | 0 | 0 |
| 485 | L | 0.4116 | 0.4116 | 0 | 0 |
| 486 | L | 0.3399 | 0.3399 | 0 | 0 |
| 487 | T | 0.3399 | 0.3399 | 0 | 0 |
| 488 | R | 0.3399 | 0.3399 | 0 | 0 |
| 489 | M | 0.3096 | 0.3096 | 0 | 0 |
| 490 | V | 0.3005 | 0.3005 | 0 | 0 |
| 491 | A | 0.3225 | 0.3225 | 0 | 0 |
| 492 | L | 0.2209 | 0.2209 | 0 | 0 |
| 493 | M | 0.1349 | 0.1349 | 0 | 0 |
| 494 | K | 0.1532 | 0.1532 | 0 | 0 |
| 495 | T | 0.0935 | 0.0935 | 0 | 0 |
| 496 | V | 0.1322 | 0.1322 | 0 | 0 |
| 497 | E | 0.1766 | 0.1766 | 0 | 0 |
| 498 | T | 0.1921 | 0.1921 | 0 | 0 |
| 499 | G | 0.1349 | 0.1349 | 0 | 0 |
| 500 | R | 0.106 | 0.106 | 0 | 0 |
| 501 | R | 0.1532 | 0.1532 | 0 | 0 |
| 502 | F | 0.1667 | 0.1667 | 0 | 0 |
| 503 | F | 0.1602 | 0.1602 | 0 | 0 |
| 504 | P | 0.1635 | 0.1635 | 0 | 0 |
| 505 | Y | 0.1456 | 0.1456 | 0 | 0 |
| 506 | G | 0.1532 | 0.1532 | 0 | 0 |
| 507 | S | 0.1456 | 0.1456 | 0 | 0 |
| 508 | E | 0.0965 | 0.0965 | 0 | 0 |
| 509 | V | 0.0935 | 0.0935 | 0 | 0 |
| 510 | L | 0.0935 | 0.0935 | 0 | 0 |
| 511 | D | 0.0991 | 0.0991 | 0 | 0 |
| 512 | K | 0.0677 | 0.0677 | 0 | 0 |
| 513 | Y | 0.0813 | 0.0813 | 0 | 0 |
| 514 | M | 0.138 | 0.138 | 0 | 0 |
| 515 | A | 0.115 | 0.115 | 0 | 0 |
| 516 | E | 0.1117 | 0.1117 | 0 | 0 |
| 517 | Y | 0.106 | 0.106 | 0 | 0 |
| 518 | I | 0.0621 | 0.0621 | 0 | 0 |
| 519 | D | 0.0587 | 0.0587 | 0 | 0 |
| 520 | D | 0.0858 | 0.0858 | 0 | 0 |
| 521 | D | 0.1349 | 0.1349 | 0 | 0 |
| 522 | I | 0.1349 | 0.1349 | 0 | 0 |
| 523 | L | 0.1349 | 0.1349 | 0 | 0 |
| 524 | D | 0.1921 | 0.1921 | 0 | 0 |
| 525 | D | 0.2292 | 0.2292 | 0 | 0 |
| 526 | F | 0.2531 | 0.2531 | 0 | 0 |
| 527 | H | 0.2333 | 0.2333 | 0 | 0 |
| 528 | F | 0.2432 | 0.2432 | 0 | 0 |
| 529 | E | 0.3005 | 0.3005 | 0 | 0 |
| 530 | K | 0.2913 | 0.2913 | 0 | 0 |
| 531 | G | 0.27 | 0.2748 | 0 | 0.0048 |
| 532 | S | 0.2657 | 0.2657 | 0 | 0 |
| 533 | T | 0.2657 | 0.27 | 0 | 0.0043 |
| 534 | H | 0.3263 | 0.3311 | 0 | 0.0048 |
| 535 | E | 0.3263 | 0.3311 | 0 | 0.0048 |
| 536 | R | 0.27 | 0.2786 | 0 | 0.0086 |
| 537 | R | 0.3578 | 0.3668 | 0 | 0.009 |
| 538 | L | 0.363 | 0.3717 | 0 | 0.0087 |
| 539 | K | 0.4379 | 0.4458 | 0 | 0.0079 |
| 540 | R | 0.3885 | 0.4037 | 0 | 0.0152 |
| 541 | M | 0.4203 | 0.4333 | 0 | 0.013 |
| 542 | R | 0.4203 | 0.4333 | 0 | 0.013 |
| 543 | Y | 0.3992 | 0.4149 | 0 | 0.0157 |
| 544 | R | 0.3578 | 0.3762 | 0 | 0.0184 |
| 545 | E | 0.3578 | 0.3806 | 0 | 0.0228 |
| 546 | L | 0.3578 | 0.3806 | 0 | 0.0228 |
| 547 | K | 0.363 | 0.3847 | 0 | 0.0217 |
| 548 | D | 0.3806 | 0.3992 | 0 | 0.0186 |
| 549 | D | 0.4379 | 0.46 | 0 | 0.0221 |
| 550 | V | 0.442 | 0.4651 | 0 | 0.0231 |
| 551 | Q | 0.46 | 0.4825 | 0 | 0.0225 |
| 552 | K | 0.4333 | 0.4513 | 0 | 0.018 |
| 553 | A | 0.4203 | 0.442 | 0 | 0.0217 |
| 554 | Y | 0.4879 | 0.5084 | 0 | 0.0205 |
| 555 | S | 0.4879 | 0.5084 | 0 | 0.0205 |
| 556 | K | 0.4458 | 0.5084 | 0 | 0.0626 |
| 557 | D | 0.4651 | 0.5229 | 0 | 0.0578 |
| 558 | K | 0.46 | 0.5126 | 0 | 0.0526 |
| 559 | E | 0.4458 | 0.5043 | 0 | 0.0585 |
| 560 | S | 0.4458 | 0.5043 | 0 | 0.0585 |
| 561 | K | 0.4918 | 0.5412 | 0 | 0.0494 |
| 562 | I | 0.4651 | 0.5173 | 0 | 0.0522 |
| 563 | A | 0.4967 | 0.5473 | 0 | 0.0506 |
| 564 | R | 0.5084 | 0.5549 | 0 | 0.0465 |
| 565 | S | 0.5549 | 0.5992 | 0 | 0.0443 |
| 566 | S | 0.5514 | 0.5941 | 0 | 0.0427 |
| 567 | L | 0.5412 | 0.5846 | 0 | 0.0434 |
| 568 | S | 0.5043 | 0.5412 | 0 | 0.0369 |
| 569 | A | 0.5084 | 0.5514 | 0 | 0.043 |
| 570 | S | 0.5126 | 0.5549 | 0 | 0.0423 |
| 571 | S | 0.5126 | 0.5549 | 0 | 0.0423 |
| 572 | S | 0.4703 | 0.5173 | 0 | 0.047 |
| 573 | P | 0.5253 | 0.5802 | 0 | 0.0549 |
| 574 | S | 0.5412 | 0.5992 | 0 | 0.058 |
| 575 | S | 0.5296 | 0.59 | 0 | 0.0604 |
| 576 | S | 0.5253 | 0.5846 | 0 | 0.0593 |
| 577 | S | 0.6079 | 0.6293 | 0 | 0.0214 |
| 578 | I | 0.6827 | 0.7034 | 0 | 0.0207 |
| 579 | R | 0.7232 | 0.7458 | 0 | 0.0226 |
| 580 | D | 0.7772 | 0.7951 | 0 | 0.0179 |
| 581 | D | 0.8158 | 0.8391 | 0 | 0.0233 |
| 582 | L | 0.8521 | 0.8713 | 0 | 0.0192 |
| 583 | H | 0.8886 | 0.904 | 0 | 0.0154 |
| 584 | N | 0.892 | 0.9068 | 0 | 0.0148 |
| 585 | T | 0.918 | 0.9316 | 0 | 0.0136 |
| 586 | T | 0.9447 | 0.9553 | 0 | 0.0106 |
| >AtNPR4 | | | | | |
| (AT4G19660) | | | | | |
| 1 | M | 0.9208 | 0.9208 | 0 | 0 |
| 2 | A | 0.8521 | 0.8521 | 0 | 0 |
| 3 | A | 0.8118 | 0.8118 | 0 | 0 |
| 4 | T | 0.6984 | 0.6984 | 0 | 0 |
| 5 | A | 0.665 | 0.665 | 0 | 0 |
| 6 | I | 0.6219 | 0.6219 | 0 | 0 |
| 7 | E | 0.5711 | 0.5711 | 0 | 0 |
| 8 | P | 0.5374 | 0.5374 | 0 | 0 |
| 9 | S | 0.442 | 0.442 | 0 | 0 |
| 10 | S | 0.3939 | 0.3939 | 0 | 0 |
| 11 | S | 0.3535 | 0.3535 | 0 | 0 |
| 12 | I | 0.4037 | 0.4037 | 0 | 0 |
| 13 | S | 0.4203 | 0.4203 | 0 | 0 |
| 14 | F | 0.4703 | 0.4703 | 0 | 0 |
| 15 | T | 0.4203 | 0.4203 | 0 | 0 |
| 16 | S | 0.3939 | 0.3939 | 0 | 0 |
| 17 | S | 0.4458 | 0.4458 | 0 | 0 |
| 18 | H | 0.4203 | 0.4245 | 0 | 0.0042 |
| 19 | L | 0.3399 | 0.3456 | 0 | 0.0057 |
| 20 | S | 0.3456 | 0.3535 | 0 | 0.0079 |
| 21 | N | 0.3456 | 0.3578 | 0 | 0.0122 |
| 22 | P | 0.3311 | 0.3456 | 0 | 0.0145 |
| 23 | S | 0.3717 | 0.3847 | 0 | 0.013 |
| 24 | P | 0.3762 | 0.3885 | 0 | 0.0123 |
| 25 | V | 0.3847 | 0.4037 | 0 | 0.019 |
| 26 | V | 0.4078 | 0.4245 | 0 | 0.0167 |
| 27 | T | 0.4379 | 0.4556 | 0 | 0.0177 |
| 28 | T | 0.3806 | 0.3992 | 0 | 0.0186 |
| 29 | Y | 0.3717 | 0.3885 | 0 | 0.0168 |
| 30 | H | 0.4282 | 0.4458 | 0 | 0.0176 |
| 31 | S | 0.4333 | 0.4556 | 0 | 0.0223 |
| 32 | A | 0.3668 | 0.3885 | 0 | 0.0217 |
| 33 | A | 0.363 | 0.3885 | 0 | 0.0255 |
| 34 | N | 0.3806 | 0.4078 | 0 | 0.0272 |
| 35 | L | 0.2963 | 0.3263 | 0 | 0.03 |
| 36 | E | 0.2913 | 0.3225 | 0 | 0.0312 |
| 37 | E | 0.3399 | 0.3762 | 0 | 0.0363 |
| 38 | L | 0.3399 | 0.3806 | 0 | 0.0407 |
| 39 | S | 0.3668 | 0.4078 | 0 | 0.041 |
| 40 | S | 0.4458 | 0.4825 | 0 | 0.0367 |
| 41 | N | 0.363 | 0.4825 | 0 | 0.1195 |
| 42 | L | 0.3806 | 0.5008 | 0 | 0.1202 |
| 43 | E | 0.3311 | 0.4513 | 0 | 0.1202 |
| 44 | Q | 0.3535 | 0.4781 | 0 | 0.1246 |
| 45 | L | 0.3668 | 0.4918 | 0 | 0.125 |
| 46 | L | 0.4078 | 0.5296 | 0 | 0.1218 |
| 47 | T | 0.4037 | 0.5253 | 0 | 0.1216 |
| 48 | N | 0.3005 | 0.4245 | 0 | 0.124 |
| 49 | P | 0.282 | 0.4078 | 0 | 0.1258 |
| 50 | D | 0.1998 | 0.3225 | 0 | 0.1227 |
| 51 | S | 0.2167 | 0.3399 | 0 | 0.1232 |
| 52 | D | 0.2255 | 0.3491 | 0 | 0.1236 |
| 53 | Y | 0.3053 | 0.4282 | 0 | 0.1229 |
| 54 | T | 0.2483 | 0.3762 | 0 | 0.1279 |
| 55 | D | 0.2333 | 0.363 | 0 | 0.1297 |
| 56 | A | 0.3263 | 0.4556 | 0 | 0.1293 |
| 57 | E | 0.3184 | 0.4458 | 0 | 0.1274 |
| 58 | I | 0.3225 | 0.4458 | 0 | 0.1233 |
| 59 | I | 0.2602 | 0.3806 | 0 | 0.1204 |
| 60 | I | 0.2255 | 0.3535 | 0 | 0.128 |
| 61 | E | 0.1998 | 0.3311 | 0 | 0.1313 |
| 62 | E | 0.2122 | 0.3263 | 0 | 0.1141 |
| 63 | E | 0.1566 | 0.2602 | 0 | 0.1036 |
| 64 | A | 0.1602 | 0.27 | 0 | 0.1098 |
| 65 | N | 0.138 | 0.2432 | 0 | 0.1052 |
| 66 | P | 0.1117 | 0.2041 | 0 | 0.0924 |
| 67 | V | 0.1292 | 0.2255 | 0 | 0.0963 |
| 68 | S | 0.115 | 0.208 | 0 | 0.093 |
| 69 | V | 0.1698 | 0.2786 | 0 | 0.1088 |
| 70 | H | 0.1667 | 0.2786 | 0 | 0.1119 |
| 71 | R | 0.1698 | 0.2865 | 0 | 0.1167 |
| 72 | S | 0.1088 | 0.2041 | 0 | 0.0953 |
| 73 | V | 0.1041 | 0.1958 | 0 | 0.0917 |
| 74 | L | 0.0621 | 0.1292 | 0 | 0.0671 |
| 75 | A | 0.0464 | 0.0991 | 0 | 0.0527 |
| 76 | A | 0.0455 | 0.0965 | 0 | 0.051 |
| 77 | R | 0.0363 | 0.0744 | 0 | 0.0381 |
| 78 | S | 0.0554 | 0.115 | 0 | 0.0596 |
| 79 | K | 0.0567 | 0.1205 | 0 | 0.0638 |
| 80 | F | 0.0884 | 0.1667 | 0 | 0.0783 |
| 81 | F | 0.0935 | 0.1635 | 0 | 0.07 |
| 82 | L | 0.0965 | 0.1698 | 0 | 0.0733 |
| 83 | D | 0.1495 | 0.1732 | 0 | 0.0237 |
| 84 | L | 0.1958 | 0.2209 | 0 | 0.0251 |
| 85 | F | 0.2558 | 0.282 | 0 | 0.0262 |
| 86 | K | 0.3146 | 0.3359 | 0 | 0.0213 |
| 87 | K | 0.3399 | 0.363 | 0 | 0.0231 |
| 88 | D | 0.2657 | 0.282 | 0 | 0.0163 |
| 89 | K | 0.282 | 0.3005 | 0 | 0.0185 |
| 90 | D | 0.2483 | 0.2657 | 0 | 0.0174 |
| 91 | S | 0.3263 | 0.3456 | 0 | 0.0193 |
| 92 | S | 0.4078 | 0.4203 | 0 | 0.0125 |
| 93 | E | 0.4037 | 0.4149 | 0 | 0.0112 |
| 94 | K | 0.3184 | 0.3263 | 0 | 0.0079 |
| 95 | K | 0.4149 | 0.4282 | 0 | 0.0133 |
| 96 | P | 0.4203 | 0.4245 | 0 | 0.0042 |
| 97 | K | 0.4245 | 0.4282 | 0 | 0.0037 |
| 98 | Y | 0.4282 | 0.4333 | 0 | 0.0051 |
| 99 | Q | 0.3535 | 0.3578 | 0 | 0.0043 |
| 100 | M | 0.3491 | 0.3535 | 0 | 0.0044 |
| 101 | K | 0.3535 | 0.3578 | 0 | 0.0043 |
| 102 | D | 0.3578 | 0.3668 | 0 | 0.009 |
| 103 | L | 0.3311 | 0.3399 | 0 | 0.0088 |
| 104 | L | 0.2432 | 0.2558 | 0 | 0.0126 |
| 105 | P | 0.1766 | 0.1844 | 0 | 0.0078 |
| 106 | Y | 0.1698 | 0.1844 | 0 | 0.0146 |
| 107 | G | 0.0858 | 0.0991 | 0 | 0.0133 |
| 108 | N | 0.0526 | 0.0621 | 0 | 0.0095 |
| 109 | V | 0.0909 | 0.1041 | 0 | 0.0132 |
| 110 | G | 0.049 | 0.0567 | 0 | 0.0077 |
| 111 | R | 0.0414 | 0.049 | 0 | 0.0076 |
| 112 | E | 0.0245 | 0.0304 | 0 | 0.0059 |
| 113 | A | 0.0231 | 0.0304 | 0 | 0.0073 |
| 114 | F | 0.0376 | 0.0478 | 0 | 0.0102 |
| 115 | L | 0.0587 | 0.0813 | 0 | 0.0226 |
| 116 | H | 0.0286 | 0.0405 | 0 | 0.0119 |
| 117 | F | 0.0455 | 0.0621 | 0 | 0.0166 |
| 118 | L | 0.0514 | 0.0744 | 0 | 0.023 |
| 119 | S | 0.0245 | 0.0376 | 0 | 0.0131 |
| 120 | Y | 0.0441 | 0.066 | 0 | 0.0219 |
| 121 | I | 0.0252 | 0.0395 | 0 | 0.0143 |
| 122 | Y | 0.0268 | 0.0405 | 0 | 0.0137 |
| 123 | T | 0.0141 | 0.0245 | 0 | 0.0104 |
| 124 | G | 0.0173 | 0.0286 | 0 | 0.0113 |
| 125 | R | 0.0308 | 0.0478 | 0 | 0.017 |
| 126 | L | 0.0231 | 0.0677 | 0 | 0.0446 |
| 127 | K | 0.0128 | 0.0414 | 0 | 0.0286 |
| 128 | P | 0.0279 | 0.0771 | 0 | 0.0492 |
| 129 | F | 0.0425 | 0.1117 | 0 | 0.0692 |
| 130 | P | 0.0252 | 0.0677 | 0 | 0.0425 |
| 131 | I | 0.0182 | 0.1041 | 0 | 0.0859 |
| 132 | E | 0.0231 | 0.124 | 0 | 0.1009 |
| 133 | V | 0.0336 | 0.1766 | 0 | 0.143 |
| 134 | S | 0.0395 | 0.1921 | 0 | 0.1526 |
| 135 | T | 0.0405 | 0.1878 | 0 | 0.1473 |
| 136 | S | 0.0194 | 0.1805 | 0 | 0.1611 |
| 137 | V | 0.0327 | 0.2483 | 0 | 0.2156 |
| 138 | D | 0.0414 | 0.282 | 0 | 0.2406 |
| 139 | S | 0.0274 | 0.2255 | 0 | 0.1981 |
| 140 | V | 0.0336 | 0.2531 | 0 | 0.2195 |
| 141 | S | 0.0316 | 0.2432 | 0 | 0.2116 |
| 142 | A | 0.0286 | 0.2292 | 0 | 0.2006 |
| 143 | H | 0.0194 | 0.1844 | 0 | 0.165 |
| 144 | D | 0.0194 | 0.1844 | 0 | 0.165 |
| 145 | S | 0.0218 | 0.1998 | 0 | 0.178 |
| 146 | S | 0.0116 | 0.1322 | 0 | 0.1206 |
| 147 | K | 0.02 | 0.1178 | 0 | 0.0978 |
| 148 | P | 0.0218 | 0.124 | 0 | 0.1022 |
| 149 | A | 0.0144 | 0.0965 | 0 | 0.0821 |
| 150 | I | 0.015 | 0.0991 | 0 | 0.0841 |
| 151 | D | 0.0128 | 0.0858 | 0 | 0.073 |
| 152 | F | 0.0179 | 0.0587 | 0 | 0.0408 |
| 153 | A | 0.0118 | 0.0414 | 0 | 0.0296 |
| 154 | V | 0.0141 | 0.0464 | 0 | 0.0323 |
| 155 | E | 0.0069 | 0.0218 | 0 | 0.0149 |
| 156 | L | 0.0081 | 0.0274 | 0 | 0.0193 |
| 157 | M | 0.0182 | 0.0308 | 0 | 0.0126 |
| 158 | Y | 0.0094 | 0.016 | 0 | 0.0066 |
| 159 | A | 0.0044 | 0.0075 | 0 | 0.0031 |
| 160 | S | 0.0066 | 0.0086 | 0 | 0.002 |
| 161 | F | 0.0097 | 0.015 | 0 | 0.0053 |
| 162 | V | 0.0035 | 0.0061 | 0 | 0.0026 |
| 163 | F | 0.0082 | 0.0118 | 0 | 0.0036 |
| 164 | Q | 0.009 | 0.0131 | 0 | 0.0041 |
| 165 | I | 0.0157 | 0.0226 | 0 | 0.0069 |
| 166 | P | 0.0069 | 0.0086 | 0 | 0.0017 |
| 167 | D | 0.0116 | 0.0157 | 0 | 0.0041 |
| 168 | L | 0.015 | 0.0212 | 0 | 0.0062 |
| 169 | V | 0.0131 | 0.0179 | 0 | 0.0048 |
| 170 | S | 0.009 | 0.0121 | 0 | 0.0031 |
| 171 | S | 0.0102 | 0.0137 | 0 | 0.0035 |
| 172 | F | 0.0231 | 0.0297 | 0 | 0.0066 |
| 173 | Q | 0.0363 | 0.0441 | 0 | 0.0078 |
| 174 | R | 0.0363 | 0.0425 | 0 | 0.0062 |
| 175 | K | 0.0194 | 0.0231 | 0 | 0.0037 |
| 176 | L | 0.0395 | 0.0464 | 0 | 0.0069 |
| 177 | R | 0.0336 | 0.0405 | 0 | 0.0069 |
| 178 | N | 0.0173 | 0.0218 | 0 | 0.0045 |
| 179 | Y | 0.0173 | 0.0226 | 0 | 0.0053 |
| 180 | V | 0.0395 | 0.049 | 0 | 0.0095 |
| 181 | E | 0.0212 | 0.0268 | 0 | 0.0056 |
| 182 | K | 0.0102 | 0.0137 | 0 | 0.0035 |
| 183 | S | 0.0131 | 0.0179 | 0 | 0.0048 |
| 184 | L | 0.0079 | 0.0097 | 0 | 0.0018 |
| 185 | V | 0.0069 | 0.0086 | 0 | 0.0017 |
| 186 | E | 0.0029 | 0.0044 | 0 | 0.0015 |
| 187 | N | 0.0069 | 0.0094 | 0 | 0.0025 |
| 188 | V | 0.0031 | 0.0099 | 0 | 0.0068 |
| 189 | L | 0.0031 | 0.0099 | 0 | 0.0068 |
| 190 | P | 0.0029 | 0.0097 | 0 | 0.0068 |
| 191 | I | 0.0061 | 0.0194 | 0 | 0.0133 |
| 192 | L | 0.0055 | 0.0182 | 0 | 0.0127 |
| 193 | L | 0.0026 | 0.009 | 0 | 0.0064 |
| 194 | V | 0.0008 | 0.0055 | 0 | 0.0047 |
| 195 | A | 0.0028 | 0.0116 | 0 | 0.0088 |
| 196 | F | 0.0055 | 0.0226 | 0 | 0.0171 |
| 197 | H | 0.0021 | 0.0212 | 0 | 0.0191 |
| 198 | S | 0.0007 | 0.0086 | 0 | 0.0079 |
| 199 | D | 0.0013 | 0.0173 | 0 | 0.016 |
| 200 | L | 0.0037 | 0.0316 | 0 | 0.0279 |
| 201 | T | 0.001 | 0.0137 | 0 | 0.0127 |
| 202 | Q | 0.0026 | 0.0231 | 0 | 0.0205 |
| 203 | L | 0.0042 | 0.0387 | 0 | 0.0345 |
| 204 | L | 0.0084 | 0.0677 | 0 | 0.0593 |
| 205 | D | 0.0157 | 0.1117 | 0 | 0.096 |
| 206 | Q | 0.009 | 0.0723 | 0 | 0.0633 |
| 207 | S | 0.0226 | 0.1416 | 0 | 0.119 |
| 208 | I | 0.0226 | 0.1349 | 0 | 0.1123 |
| 209 | E | 0.0245 | 0.0771 | 0 | 0.0526 |
| 210 | R | 0.0102 | 0.0771 | 0 | 0.0669 |
| 211 | V | 0.0118 | 0.0813 | 0 | 0.0695 |
| 212 | A | 0.0124 | 0.0832 | 0 | 0.0708 |
| 213 | R | 0.0118 | 0.0771 | 0 | 0.0653 |
| 214 | S | 0.0259 | 0.1349 | 0 | 0.109 |
| 215 | D | 0.0279 | 0.1456 | 0 | 0.1177 |
| 216 | L | 0.0327 | 0.1635 | 0 | 0.1308 |
| 217 | D | 0.0179 | 0.1041 | 0 | 0.0862 |
| 218 | R | 0.0387 | 0.1041 | 0 | 0.0654 |
| 219 | F | 0.0502 | 0.1322 | 0 | 0.082 |
| 220 | S | 0.0304 | 0.0832 | 0 | 0.0528 |
| 221 | I | 0.0327 | 0.0909 | 0 | 0.0582 |
| 222 | E | 0.0526 | 0.1349 | 0 | 0.0823 |
| 223 | K | 0.0395 | 0.1041 | 0 | 0.0646 |
| 224 | E | 0.0425 | 0.106 | 0 | 0.0635 |
| 225 | L | 0.0414 | 0.1117 | 0 | 0.0703 |
| 226 | P | 0.0245 | 0.0643 | 0 | 0.0398 |
| 227 | L | 0.0455 | 0.115 | 0 | 0.0695 |
| 228 | E | 0.0286 | 0.0744 | 0 | 0.0458 |
| 229 | V | 0.0286 | 0.0723 | 0 | 0.0437 |
| 230 | L | 0.0542 | 0.1292 | 0 | 0.075 |
| 231 | E | 0.0701 | 0.0884 | 0 | 0.0183 |
| 232 | K | 0.124 | 0.1495 | 0 | 0.0255 |
| 233 | I | 0.0723 | 0.0909 | 0 | 0.0186 |
| 234 | K | 0.0935 | 0.115 | 0 | 0.0215 |
| 235 | Q | 0.0909 | 0.1117 | 0 | 0.0208 |
| 236 | L | 0.1041 | 0.1205 | 0 | 0.0164 |
| 237 | R | 0.0832 | 0.0991 | 0 | 0.0159 |
| 238 | V | 0.1416 | 0.1602 | 0 | 0.0186 |
| 239 | K | 0.1349 | 0.1532 | 0 | 0.0183 |
| 240 | S | 0.1878 | 0.208 | 0 | 0.0202 |
| 241 | V | 0.1732 | 0.1844 | 0 | 0.0112 |
| 242 | N | 0.1766 | 0.1921 | 0 | 0.0155 |
| 243 | I | 0.1766 | 0.1878 | 0 | 0.0112 |
| 244 | P | 0.2483 | 0.2602 | 0 | 0.0119 |
| 245 | E | 0.2657 | 0.2748 | 0 | 0.0091 |
| 246 | V | 0.2483 | 0.2558 | 0 | 0.0075 |
| 247 | E | 0.2657 | 0.2748 | 0 | 0.0091 |
| 248 | D | 0.1998 | 0.2041 | 0 | 0.0043 |
| 249 | K | 0.2531 | 0.2558 | 0 | 0.0027 |
| 250 | S | 0.2385 | 0.2432 | 0 | 0.0047 |
| 251 | I | 0.1635 | 0.1667 | 0 | 0.0032 |
| 252 | E | 0.2255 | 0.2292 | 0 | 0.0037 |
| 253 | R | 0.2255 | 0.2255 | 0 | 0 |
| 254 | T | 0.3146 | 0.3184 | 0 | 0.0038 |
| 255 | G | 0.2913 | 0.2913 | 0 | 0 |
| 256 | K | 0.2255 | 0.2255 | 0 | 0 |
| 257 | V | 0.3096 | 0.3096 | 0 | 0 |
| 258 | L | 0.2255 | 0.2255 | 0 | 0 |
| 259 | K | 0.1698 | 0.1698 | 0 | 0 |
| 260 | A | 0.1635 | 0.1635 | 0 | 0 |
| 261 | L | 0.1088 | 0.1088 | 0 | 0 |
| 262 | D | 0.124 | 0.124 | 0 | 0 |
| 263 | S | 0.0744 | 0.0744 | 0 | 0 |
| 264 | D | 0.0771 | 0.0771 | 0 | 0 |
| 265 | D | 0.0858 | 0.0858 | 0 | 0 |
| 266 | V | 0.0832 | 0.0832 | 0 | 0 |
| 267 | E | 0.0965 | 0.0965 | 0 | 0 |
| 268 | L | 0.0744 | 0.0744 | 0 | 0 |
| 269 | V | 0.115 | 0.115 | 0 | 0 |
| 270 | K | 0.0701 | 0.0701 | 0 | 0 |
| 271 | L | 0.0858 | 0.0858 | 0 | 0 |
| 272 | L | 0.138 | 0.138 | 0 | 0 |
| 273 | L | 0.1088 | 0.1088 | 0 | 0 |
| 274 | T | 0.1117 | 0.1117 | 0 | 0 |
| 275 | E | 0.1088 | 0.1088 | 0 | 0 |
| 276 | S | 0.0567 | 0.0567 | 0 | 0 |
| 277 | D | 0.0858 | 0.0858 | 0 | 0 |
| 278 | I | 0.0502 | 0.0502 | 0 | 0 |
| 279 | T | 0.0677 | 0.0677 | 0 | 0 |
| 280 | L | 0.0643 | 0.0643 | 0 | 0 |
| 281 | D | 0.0567 | 0.0567 | 0 | 0 |
| 282 | Q | 0.0567 | 0.0567 | 0 | 0 |
| 283 | A | 0.0909 | 0.0909 | 0 | 0 |
| 284 | N | 0.1416 | 0.1416 | 0 | 0 |
| 285 | G | 0.1766 | 0.1766 | 0 | 0 |
| 286 | L | 0.1602 | 0.1602 | 0 | 0 |
| 287 | H | 0.1117 | 0.1117 | 0 | 0 |
| 288 | Y | 0.0723 | 0.0723 | 0 | 0 |
| 289 | A | 0.1266 | 0.1266 | 0 | 0 |
| 290 | V | 0.1416 | 0.1416 | 0 | 0 |
| 291 | A | 0.1667 | 0.1667 | 0 | 0 |
| 292 | Y | 0.1018 | 0.1018 | 0 | 0 |
| 293 | S | 0.1041 | 0.1041 | 0 | 0 |
| 294 | D | 0.0789 | 0.0789 | 0 | 0 |
| 295 | P | 0.0789 | 0.0789 | 0 | 0 |
| 296 | K | 0.0607 | 0.0607 | 0 | 0 |
| 297 | V | 0.0858 | 0.0858 | 0 | 0 |
| 298 | V | 0.0935 | 0.0935 | 0 | 0 |
| 299 | T | 0.0909 | 0.0909 | 0 | 0 |
| 300 | Q | 0.1117 | 0.1117 | 0 | 0 |
| 301 | V | 0.0832 | 0.0832 | 0 | 0 |
| 302 | L | 0.1041 | 0.1041 | 0 | 0 |
| 303 | D | 0.1566 | 0.1566 | 0 | 0 |
| 304 | L | 0.1566 | 0.1566 | 0 | 0 |
| 305 | D | 0.1532 | 0.1532 | 0 | 0 |
| 306 | M | 0.1266 | 0.1266 | 0 | 0 |
| 307 | A | 0.0832 | 0.0832 | 0 | 0 |
| 308 | D | 0.1205 | 0.1205 | 0 | 0 |
| 309 | V | 0.1205 | 0.1205 | 0 | 0 |
| 310 | N | 0.0771 | 0.0771 | 0 | 0 |
| 311 | F | 0.0723 | 0.0723 | 0 | 0 |
| 312 | R | 0.0587 | 0.0587 | 0 | 0 |
| 313 | N | 0.0832 | 0.0832 | 0 | 0 |
| 314 | S | 0.0643 | 0.0643 | 0 | 0 |
| 315 | R | 0.0832 | 0.0832 | 0 | 0 |
| 316 | G | 0.0789 | 0.0789 | 0 | 0 |
| 317 | Y | 0.1041 | 0.1041 | 0 | 0 |
| 318 | T | 0.1349 | 0.1349 | 0 | 0 |
| 319 | V | 0.1566 | 0.1566 | 0 | 0 |
| 320 | L | 0.2167 | 0.2167 | 0 | 0 |
| 321 | H | 0.1456 | 0.1456 | 0 | 0 |
| 322 | I | 0.1766 | 0.1766 | 0 | 0 |
| 323 | A | 0.1178 | 0.1178 | 0 | 0 |
| 324 | A | 0.1349 | 0.138 | 0 | 0.0031 |
| 325 | M | 0.0858 | 0.0858 | 0 | 0 |
| 326 | R | 0.0514 | 0.0526 | 0 | 0.0012 |
| 327 | R | 0.0567 | 0.0587 | 0 | 0.002 |
| 328 | E | 0.0991 | 0.1018 | 0 | 0.0027 |
| 329 | P | 0.0991 | 0.106 | 0 | 0.0069 |
| 330 | T | 0.1178 | 0.1266 | 0 | 0.0088 |
| 331 | I | 0.1805 | 0.1921 | 0 | 0.0116 |
| 332 | I | 0.1495 | 0.1635 | 0 | 0.014 |
| 333 | I | 0.2333 | 0.2531 | 0 | 0.0198 |
| 334 | P | 0.2657 | 0.282 | 0 | 0.0163 |
| 335 | L | 0.2167 | 0.2333 | 0 | 0.0166 |
| 336 | I | 0.2531 | 0.2748 | 0 | 0.0217 |
| 337 | Q | 0.1844 | 0.1998 | 0 | 0.0154 |
| 338 | K | 0.1766 | 0.1958 | 0 | 0.0192 |
| 339 | G | 0.1698 | 0.1878 | 0 | 0.018 |
| 340 | A | 0.1495 | 0.1667 | 0 | 0.0172 |
| 341 | N | 0.1532 | 0.1698 | 0 | 0.0166 |
| 342 | A | 0.1998 | 0.2255 | 0 | 0.0257 |
| 343 | S | 0.2167 | 0.2432 | 0 | 0.0265 |
| 344 | D | 0.2963 | 0.3263 | 0 | 0.03 |
| 345 | F | 0.2041 | 0.2292 | 0 | 0.0251 |
| 346 | T | 0.2041 | 0.3096 | 0 | 0.1055 |
| 347 | F | 0.282 | 0.3939 | 0 | 0.1119 |
| 348 | D | 0.2748 | 0.3806 | 0 | 0.1058 |
| 349 | G | 0.2041 | 0.3146 | 0 | 0.1105 |
| 350 | R | 0.1958 | 0.3053 | 0 | 0.1095 |
| 351 | S | 0.2292 | 0.3399 | 0 | 0.1107 |
| 352 | A | 0.2657 | 0.3717 | 0 | 0.106 |
| 353 | V | 0.2963 | 0.4078 | 0 | 0.1115 |
| 354 | N | 0.2865 | 0.3992 | 0 | 0.1127 |
| 355 | I | 0.2292 | 0.3456 | 0 | 0.1164 |
| 356 | S | 0.3096 | 0.4282 | 0 | 0.1186 |
| 357 | R | 0.3096 | 0.4333 | 0 | 0.1237 |
| 358 | R | 0.3885 | 0.5126 | 0 | 0.1241 |
| 359 | L | 0.3939 | 0.5173 | 0 | 0.1234 |
| 360 | T | 0.3939 | 0.5126 | 0 | 0.1187 |
| 361 | R | 0.3847 | 0.5084 | 0 | 0.1237 |
| 362 | P | 0.3847 | 0.5084 | 0 | 0.1237 |
| 363 | K | 0.3992 | 0.5229 | 0 | 0.1237 |
| 364 | D | 0.4781 | 0.6035 | 0 | 0.1254 |
| 365 | Y | 0.4825 | 0.5992 | 0 | 0.1167 |
| 366 | H | 0.5473 | 0.665 | 0 | 0.1177 |
| 367 | T | 0.5667 | 0.6219 | 0 | 0.0552 |
| 368 | K | 0.5623 | 0.6174 | 0 | 0.0551 |
| 369 | T | 0.5126 | 0.5623 | 0 | 0.0497 |
| 370 | S | 0.4879 | 0.6293 | 0 | 0.1414 |
| 371 | R | 0.4458 | 0.59 | 0 | 0.1442 |
| 372 | K | 0.4379 | 0.5802 | 0 | 0.1423 |
| 373 | E | 0.3668 | 0.5126 | 0 | 0.1458 |
| 374 | P | 0.3225 | 0.4703 | 0 | 0.1478 |
| 375 | S | 0.3311 | 0.4781 | 0 | 0.147 |
| 376 | K | 0.3992 | 0.5412 | 0 | 0.142 |
| 377 | Y | 0.4078 | 0.5374 | 0 | 0.1296 |
| 378 | R | 0.3535 | 0.4825 | 0 | 0.129 |
| 379 | L | 0.3491 | 0.4781 | 0 | 0.129 |
| 380 | S | 0.3491 | 0.4781 | 0 | 0.129 |
| 381 | I | 0.3456 | 0.4825 | 0 | 0.1369 |
| 382 | D | 0.3762 | 0.5126 | 0 | 0.1364 |
| 383 | I | 0.3146 | 0.4513 | 0 | 0.1367 |
| 384 | L | 0.27 | 0.4078 | 0 | 0.1378 |
| 385 | E | 0.2531 | 0.3847 | 0 | 0.1316 |
| 386 | R | 0.2483 | 0.3885 | 0 | 0.1402 |
| 387 | E | 0.2531 | 0.3939 | 0 | 0.1408 |
| 388 | I | 0.3146 | 0.4513 | 0 | 0.1367 |
| 389 | R | 0.3399 | 0.4781 | 0 | 0.1382 |
| 390 | R | 0.3939 | 0.5253 | 0 | 0.1314 |
| 391 | N | 0.4037 | 0.5253 | 0 | 0.1216 |
| 392 | P | 0.4556 | 0.5762 | 0 | 0.1206 |
| 393 | L | 0.442 | 0.5711 | 0 | 0.1291 |
| 394 | V | 0.4879 | 0.6174 | 0 | 0.1295 |
| 395 | S | 0.5008 | 0.6255 | 0 | 0.1247 |
| 396 | G | 0.5296 | 0.6516 | 0 | 0.122 |
| 397 | D | 0.5331 | 0.6516 | 0 | 0.1185 |
| 398 | T | 0.5412 | 0.6604 | 0 | 0.1192 |
| 399 | P | 0.5173 | 0.6442 | 0 | 0.1269 |
| 400 | T | 0.5331 | 0.6557 | 0 | 0.1226 |
| 401 | S | 0.5008 | 0.6293 | 0 | 0.1285 |
| 402 | S | 0.4967 | 0.6219 | 0 | 0.1252 |
| 403 | H | 0.3939 | 0.5229 | 0 | 0.129 |
| 404 | S | 0.3885 | 0.5126 | 0 | 0.1241 |
| 405 | M | 0.363 | 0.4918 | 0 | 0.1288 |
| 406 | P | 0.282 | 0.4116 | 0 | 0.1296 |
| 407 | E | 0.3005 | 0.4245 | 0 | 0.124 |
| 408 | D | 0.2913 | 0.4116 | 0 | 0.1203 |
| 409 | L | 0.282 | 0.4078 | 0 | 0.1258 |
| 410 | Q | 0.1958 | 0.3146 | 0 | 0.1188 |
| 411 | M | 0.1921 | 0.3146 | 0 | 0.1225 |
| 412 | R | 0.208 | 0.2483 | 0 | 0.0403 |
| 413 | L | 0.1766 | 0.2167 | 0 | 0.0401 |
| 414 | L | 0.1958 | 0.2333 | 0 | 0.0375 |
| 415 | Y | 0.1322 | 0.1667 | 0 | 0.0345 |
| 416 | L | 0.0965 | 0.1266 | 0 | 0.0301 |
| 417 | E | 0.0441 | 0.0587 | 0 | 0.0146 |
| 418 | K | 0.0478 | 0.0643 | 0 | 0.0165 |
| 419 | R | 0.0363 | 0.049 | 0 | 0.0127 |
| 420 | V | 0.0643 | 0.0909 | 0 | 0.0266 |
| 421 | G | 0.049 | 0.066 | 0 | 0.017 |
| 422 | L | 0.0607 | 0.0832 | 0 | 0.0225 |
| 423 | A | 0.0414 | 0.0554 | 0 | 0.014 |
| 424 | Q | 0.0542 | 0.0744 | 0 | 0.0202 |
| 425 | L | 0.066 | 0.0909 | 0 | 0.0249 |
| 426 | F | 0.1178 | 0.1495 | 0 | 0.0317 |
| 427 | F | 0.124 | 0.1566 | 0 | 0.0326 |
| 428 | P | 0.0909 | 0.1205 | 0 | 0.0296 |
| 429 | A | 0.0965 | 0.1266 | 0 | 0.0301 |
| 430 | E | 0.066 | 0.0909 | 0 | 0.0249 |
| 431 | A | 0.1088 | 0.1416 | 0 | 0.0328 |
| 432 | N | 0.1117 | 0.1416 | 0 | 0.0299 |
| 433 | V | 0.1566 | 0.1921 | 0 | 0.0355 |
| 434 | A | 0.1844 | 0.2209 | 0 | 0.0365 |
| 435 | M | 0.1998 | 0.2292 | 0 | 0.0294 |
| 436 | D | 0.2041 | 0.3053 | 0 | 0.1012 |
| 437 | V | 0.2963 | 0.3885 | 0 | 0.0922 |
| 438 | A | 0.3885 | 0.4825 | 0 | 0.094 |
| 439 | N | 0.3096 | 0.4078 | 0 | 0.0982 |
| 440 | V | 0.2963 | 0.3939 | 0 | 0.0976 |
| 441 | E | 0.2748 | 0.3717 | 0 | 0.0969 |
| 442 | G | 0.3311 | 0.4282 | 0 | 0.0971 |
| 443 | T | 0.3535 | 0.4513 | 0 | 0.0978 |
| 444 | S | 0.4458 | 0.5374 | 0 | 0.0916 |
| 445 | E | 0.4825 | 0.5667 | 0 | 0.0842 |
| 446 | S | 0.5173 | 0.6079 | 0 | 0.0906 |
| 447 | T | 0.5126 | 0.6035 | 0 | 0.0909 |
| 448 | G | 0.5711 | 0.6604 | 0 | 0.0893 |
| 449 | L | 0.6035 | 0.687 | 0 | 0.0835 |
| 450 | L | 0.6174 | 0.6944 | 0 | 0.077 |
| 451 | T | 0.6715 | 0.754 | 0 | 0.0825 |
| 452 | P | 0.6124 | 0.6944 | 0 | 0.082 |
| 453 | P | 0.6124 | 0.6944 | 0 | 0.082 |
| 454 | P | 0.6079 | 0.6906 | 0 | 0.0827 |
| 455 | S | 0.5583 | 0.6474 | 0 | 0.0891 |
| 456 | N | 0.5374 | 0.6334 | 0 | 0.096 |
| 457 | D | 0.5623 | 0.59 | 0 | 0.0277 |
| 458 | T | 0.5667 | 0.5992 | 0 | 0.0325 |
| 459 | T | 0.5711 | 0.5992 | 0 | 0.0281 |
| 460 | E | 0.6219 | 0.6442 | 0 | 0.0223 |
| 461 | N | 0.687 | 0.7111 | 0 | 0.0241 |
| 462 | L | 0.6374 | 0.6557 | 0 | 0.0183 |
| 463 | G | 0.5623 | 0.59 | 0 | 0.0277 |
| 464 | K | 0.5549 | 0.5802 | 0 | 0.0253 |
| 465 | V | 0.5296 | 0.5549 | 0 | 0.0253 |
| 466 | D | 0.5229 | 0.5514 | 0 | 0.0285 |
| 467 | L | 0.5229 | 0.5473 | 0 | 0.0244 |
| 468 | N | 0.4879 | 0.5126 | 0 | 0.0247 |
| 469 | E | 0.4333 | 0.46 | 0 | 0.0267 |
| 470 | T | 0.4282 | 0.46 | 0 | 0.0318 |
| 471 | P | 0.4203 | 0.4458 | 0 | 0.0255 |
| 472 | Y | 0.3806 | 0.4078 | 0 | 0.0272 |
| 473 | V | 0.4203 | 0.4458 | 0 | 0.0255 |
| 474 | Q | 0.3939 | 0.4149 | 0 | 0.021 |
| 475 | T | 0.3359 | 0.363 | 0 | 0.0271 |
| 476 | K | 0.3456 | 0.3717 | 0 | 0.0261 |
| 477 | R | 0.3359 | 0.363 | 0 | 0.0271 |
| 478 | M | 0.3717 | 0.3992 | 0 | 0.0275 |
| 479 | L | 0.3146 | 0.3359 | 0 | 0.0213 |
| 480 | T | 0.3146 | 0.3359 | 0 | 0.0213 |
| 481 | R | 0.3096 | 0.3359 | 0 | 0.0263 |
| 482 | M | 0.282 | 0.3096 | 0 | 0.0276 |
| 483 | K | 0.3456 | 0.3717 | 0 | 0.0261 |
| 484 | A | 0.3939 | 0.4203 | 0 | 0.0264 |
| 485 | L | 0.3005 | 0.3263 | 0 | 0.0258 |
| 486 | M | 0.2167 | 0.2432 | 0 | 0.0265 |
| 487 | K | 0.2432 | 0.2748 | 0 | 0.0316 |
| 488 | T | 0.2483 | 0.27 | 0 | 0.0217 |
| 489 | V | 0.2209 | 0.3005 | 0 | 0.0796 |
| 490 | E | 0.1998 | 0.2786 | 0 | 0.0788 |
| 491 | T | 0.208 | 0.2913 | 0 | 0.0833 |
| 492 | G | 0.1635 | 0.2333 | 0 | 0.0698 |
| 493 | R | 0.1495 | 0.2167 | 0 | 0.0672 |
| 494 | R | 0.1602 | 0.2333 | 0 | 0.0731 |
| 495 | Y | 0.1766 | 0.2558 | 0 | 0.0792 |
| 496 | F | 0.1766 | 0.2531 | 0 | 0.0765 |
| 497 | P | 0.1844 | 0.2657 | 0 | 0.0813 |
| 498 | S | 0.1958 | 0.2748 | 0 | 0.079 |
| 499 | S | 0.208 | 0.2913 | 0 | 0.0833 |
| 500 | Y | 0.2041 | 0.2865 | 0 | 0.0824 |
| 501 | E | 0.1732 | 0.2531 | 0 | 0.0799 |
| 502 | V | 0.1844 | 0.2602 | 0 | 0.0758 |
| 503 | L | 0.1844 | 0.2602 | 0 | 0.0758 |
| 504 | D | 0.1844 | 0.2602 | 0 | 0.0758 |
| 505 | K | 0.138 | 0.2041 | 0 | 0.0661 |
| 506 | Y | 0.2209 | 0.3005 | 0 | 0.0796 |
| 507 | M | 0.3146 | 0.3939 | 0 | 0.0793 |
| 508 | D | 0.2558 | 0.3359 | 0 | 0.0801 |
| 509 | Q | 0.2558 | 0.3359 | 0 | 0.0801 |
| 510 | Y | 0.2531 | 0.27 | 0 | 0.0169 |
| 511 | M | 0.3491 | 0.3717 | 0 | 0.0226 |
| 512 | D | 0.3456 | 0.3668 | 0 | 0.0212 |
| 513 | E | 0.3939 | 0.4116 | 0 | 0.0177 |
| 514 | E | 0.442 | 0.4651 | 0 | 0.0231 |
| 515 | I | 0.4282 | 0.4513 | 0 | 0.0231 |
| 516 | P | 0.3939 | 0.4116 | 0 | 0.0177 |
| 517 | D | 0.4556 | 0.4749 | 0 | 0.0193 |
| 518 | M | 0.4825 | 0.5008 | 0 | 0.0183 |
| 519 | S | 0.4825 | 0.5008 | 0 | 0.0183 |
| 520 | Y | 0.4749 | 0.4879 | 0 | 0.013 |
| 521 | P | 0.5473 | 0.5623 | 0 | 0.015 |
| 522 | E | 0.5711 | 0.5846 | 0 | 0.0135 |
| 523 | K | 0.5623 | 0.5802 | 0 | 0.0179 |
| 524 | G | 0.5473 | 0.5623 | 0 | 0.015 |
| 525 | T | 0.5514 | 0.5667 | 0 | 0.0153 |
| 526 | V | 0.5514 | 0.5667 | 0 | 0.0153 |
| 527 | K | 0.5374 | 0.5549 | 0 | 0.0175 |
| 528 | E | 0.5229 | 0.5412 | 0 | 0.0183 |
| 529 | R | 0.5173 | 0.5374 | 0 | 0.0201 |
| 530 | R | 0.5173 | 0.5374 | 0 | 0.0201 |
| 531 | Q | 0.5802 | 0.6035 | 0 | 0.0233 |
| 532 | K | 0.5549 | 0.5762 | 0 | 0.0213 |
| 533 | R | 0.5173 | 0.5374 | 0 | 0.0201 |
| 534 | M | 0.5253 | 0.5473 | 0 | 0.022 |
| 535 | R | 0.5296 | 0.5514 | 0 | 0.0218 |
| 536 | Y | 0.5229 | 0.5412 | 0 | 0.0183 |
| 537 | N | 0.5126 | 0.5331 | 0 | 0.0205 |
| 538 | E | 0.5126 | 0.5374 | 0 | 0.0248 |
| 539 | L | 0.5229 | 0.5412 | 0 | 0.0183 |
| 540 | K | 0.5173 | 0.5374 | 0 | 0.0201 |
| 541 | N | 0.5173 | 0.5412 | 0 | 0.0239 |
| 542 | D | 0.4703 | 0.4918 | 0 | 0.0215 |
| 543 | V | 0.4513 | 0.4781 | 0 | 0.0268 |
| 544 | K | 0.46 | 0.4879 | 0 | 0.0279 |
| 545 | K | 0.4781 | 0.5043 | 0 | 0.0262 |
| 546 | A | 0.4282 | 0.5008 | 0 | 0.0726 |
| 547 | Y | 0.4379 | 0.5084 | 0 | 0.0705 |
| 548 | S | 0.4282 | 0.5008 | 0 | 0.0726 |
| 549 | K | 0.4379 | 0.5043 | 0 | 0.0664 |
| 550 | D | 0.4781 | 0.5374 | 0 | 0.0593 |
| 551 | K | 0.4703 | 0.5296 | 0 | 0.0593 |
| 552 | V | 0.4879 | 0.5412 | 0 | 0.0533 |
| 553 | A | 0.4651 | 0.5253 | 0 | 0.0602 |
| 554 | R | 0.5043 | 0.5583 | 0 | 0.054 |
| 555 | S | 0.4967 | 0.5514 | 0 | 0.0547 |
| 556 | S | 0.4513 | 0.5084 | 0 | 0.0571 |
| 557 | L | 0.4781 | 0.5374 | 0 | 0.0593 |
| 558 | S | 0.5296 | 0.5846 | 0 | 0.055 |
| 559 | S | 0.5084 | 0.5667 | 0 | 0.0583 |
| 560 | S | 0.46 | 0.5253 | 0 | 0.0653 |
| 561 | S | 0.4651 | 0.5296 | 0 | 0.0645 |
| 562 | P | 0.46 | 0.5296 | 0 | 0.0696 |
| 563 | A | 0.5253 | 0.5941 | 0 | 0.0688 |
| 564 | S | 0.5374 | 0.6079 | 0 | 0.0705 |
| 565 | S | 0.5711 | 0.6374 | 0 | 0.0663 |
| 566 | L | 0.6174 | 0.6756 | 0 | 0.0582 |
| 567 | R | 0.6944 | 0.7147 | 0 | 0.0203 |
| 568 | E | 0.7688 | 0.7912 | 0 | 0.0224 |
| 569 | A | 0.8118 | 0.8311 | 0 | 0.0193 |
| 570 | L | 0.8488 | 0.865 | 0 | 0.0162 |
| 571 | E | 0.8781 | 0.8984 | 0 | 0.0203 |
| 572 | N | 0.9119 | 0.9235 | 0 | 0.0116 |
| 573 | P | 0.9208 | 0.9316 | 0 | 0.0108 |
| 574 | T | 0.9534 | 0.9624 | 0 | 0.009 |
| >AtNPR5 | | | | | |
| (AT2G41370) | | | | | |
| 1 | M | 0.7724 | 0.7724 | 0 | 0 |
| 2 | S | 0.7342 | 0.7342 | 0 | 0 |
| 3 | N | 0.6079 | 0.6079 | 0 | 0 |
| 4 | L | 0.5711 | 0.5711 | 0 | 0 |
| 5 | E | 0.4245 | 0.4245 | 0 | 0 |
| 6 | E | 0.2963 | 0.2963 | 0 | 0 |
| 7 | S | 0.2531 | 0.2558 | 0 | 0.0027 |
| 8 | L | 0.1349 | 0.138 | 0 | 0.0031 |
| 9 | R | 0.0607 | 0.0621 | 0 | 0.0014 |
| 10 | S | 0.0252 | 0.0268 | 0 | 0.0016 |
| 11 | L | 0.0179 | 0.0194 | 0 | 0.0015 |
| 12 | S | 0.0252 | 0.0268 | 0 | 0.0016 |
| 13 | L | 0.0268 | 0.0297 | 0 | 0.0029 |
| 14 | D | 0.0194 | 0.0226 | 0 | 0.0032 |
| 15 | F | 0.0173 | 0.02 | 0 | 0.0027 |
| 16 | L | 0.0144 | 0.0167 | 0 | 0.0023 |
| 17 | N | 0.0128 | 0.0144 | 0 | 0.0016 |
| 18 | L | 0.0078 | 0.0083 | 0 | 0.0005 |
| 19 | L | 0.0144 | 0.0173 | 0 | 0.0029 |
| 20 | I | 0.007 | 0.0082 | 0 | 0.0012 |
| 21 | N | 0.007 | 0.0081 | 0 | 0.0011 |
| 22 | G | 0.0075 | 0.0083 | 0 | 0.0008 |
| 23 | Q | 0.0079 | 0.0087 | 0 | 0.0008 |
| 24 | A | 0.015 | 0.0194 | 0 | 0.0044 |
| 25 | F | 0.0131 | 0.0173 | 0 | 0.0042 |
| 26 | S | 0.0137 | 0.0182 | 0 | 0.0045 |
| 27 | D | 0.0144 | 0.02 | 0 | 0.0056 |
| 28 | V | 0.0124 | 0.0179 | 0 | 0.0055 |
| 29 | T | 0.0182 | 0.0274 | 0 | 0.0092 |
| 30 | F | 0.0308 | 0.0455 | 0 | 0.0147 |
| 31 | S | 0.0567 | 0.0832 | 0 | 0.0265 |
| 32 | V | 0.0297 | 0.0789 | 0 | 0.0492 |
| 33 | E | 0.016 | 0.0464 | 0 | 0.0304 |
| 34 | G | 0.0083 | 0.0268 | 0 | 0.0185 |
| 35 | R | 0.0084 | 0.0274 | 0 | 0.019 |
| 36 | L | 0.0157 | 0.0464 | 0 | 0.0307 |
| 37 | V | 0.016 | 0.0478 | 0 | 0.0318 |
| 38 | H | 0.0157 | 0.0464 | 0 | 0.0307 |
| 39 | A | 0.0167 | 0.0502 | 0 | 0.0335 |
| 40 | H | 0.0086 | 0.0297 | 0 | 0.0211 |
| 41 | R | 0.0116 | 0.0376 | 0 | 0.026 |
| 42 | S | 0.0118 | 0.0395 | 0 | 0.0277 |
| 43 | I | 0.0245 | 0.0701 | 0 | 0.0456 |
| 44 | L | 0.0118 | 0.0395 | 0 | 0.0277 |
| 45 | A | 0.0074 | 0.0231 | 0 | 0.0157 |
| 46 | A | 0.0035 | 0.0252 | 0 | 0.0217 |
| 47 | R | 0.0082 | 0.049 | 0 | 0.0408 |
| 48 | S | 0.0144 | 0.0832 | 0 | 0.0688 |
| 49 | L | 0.0173 | 0.0909 | 0 | 0.0736 |
| 50 | F | 0.0252 | 0.1117 | 0 | 0.0865 |
| 51 | F | 0.0316 | 0.1349 | 0 | 0.1033 |
| 52 | R | 0.0363 | 0.1532 | 0 | 0.1169 |
| 53 | K | 0.0813 | 0.1766 | 0 | 0.0953 |
| 54 | F | 0.0935 | 0.1998 | 0 | 0.1063 |
| 55 | F | 0.1456 | 0.2786 | 0 | 0.133 |
| 56 | S | 0.1732 | 0.3146 | 0 | 0.1414 |
| 57 | G | 0.1532 | 0.282 | 0 | 0.1288 |
| 58 | T | 0.1635 | 0.2913 | 0 | 0.1278 |
| 59 | D | 0.1844 | 0.3225 | 0 | 0.1381 |
| 60 | S | 0.2558 | 0.3939 | 0 | 0.1381 |
| 61 | P | 0.363 | 0.5008 | 0 | 0.1378 |
| 62 | Q | 0.4458 | 0.5802 | 0 | 0.1344 |
| 63 | P | 0.4458 | 0.5846 | 0 | 0.1388 |
| 64 | V | 0.4458 | 0.5802 | 0 | 0.1344 |
| 65 | T | 0.4703 | 0.6035 | 0 | 0.1332 |
| 66 | G | 0.5711 | 0.6944 | 0 | 0.1233 |
| 67 | I | 0.6334 | 0.6715 | 0 | 0.0381 |
| 68 | D | 0.6293 | 0.665 | 0 | 0.0357 |
| 69 | P | 0.6516 | 0.6827 | 0 | 0.0311 |
| 70 | T | 0.6442 | 0.6756 | 0 | 0.0314 |
| 71 | Q | 0.6474 | 0.6827 | 0 | 0.0353 |
| 72 | H | 0.6293 | 0.665 | 0 | 0.0357 |
| 73 | G | 0.6174 | 0.6442 | 0 | 0.0268 |
| 74 | S | 0.59 | 0.6219 | 0 | 0.0319 |
| 75 | V | 0.6174 | 0.6412 | 0 | 0.0238 |
| 76 | P | 0.6412 | 0.665 | 0 | 0.0238 |
| 77 | A | 0.6079 | 0.6255 | 0 | 0.0176 |
| 78 | S | 0.665 | 0.6789 | 0 | 0.0139 |
| 79 | P | 0.5711 | 0.5941 | 0 | 0.023 |
| 80 | T | 0.4703 | 0.4879 | 0 | 0.0176 |
| 81 | R | 0.4967 | 0.5126 | 0 | 0.0159 |
| 82 | G | 0.3992 | 0.4116 | 0 | 0.0124 |
| 83 | S | 0.4078 | 0.4149 | 0 | 0.0071 |
| 84 | T | 0.4037 | 0.4078 | 0 | 0.0041 |
| 85 | A | 0.3311 | 0.3399 | 0 | 0.0088 |
| 86 | P | 0.3992 | 0.4078 | 0 | 0.0086 |
| 87 | A | 0.2963 | 0.3005 | 0 | 0.0042 |
| 88 | G | 0.3399 | 0.3456 | 0 | 0.0057 |
| 89 | I | 0.2748 | 0.2786 | 0 | 0.0038 |
| 90 | I | 0.1732 | 0.1766 | 0 | 0.0034 |
| 91 | P | 0.1266 | 0.1292 | 0 | 0.0026 |
| 92 | V | 0.0858 | 0.0884 | 0 | 0.0026 |
| 93 | N | 0.0542 | 0.0567 | 0 | 0.0025 |
| 94 | S | 0.0363 | 0.0387 | 0 | 0.0024 |
| 95 | V | 0.0376 | 0.0414 | 0 | 0.0038 |
| 96 | G | 0.0274 | 0.0308 | 0 | 0.0034 |
| 97 | Y | 0.0124 | 0.015 | 0 | 0.0026 |
| 98 | E | 0.0087 | 0.0109 | 0 | 0.0022 |
| 99 | V | 0.0087 | 0.0102 | 0 | 0.0015 |
| 100 | F | 0.016 | 0.0212 | 0 | 0.0052 |
| 101 | L | 0.0308 | 0.0414 | 0 | 0.0106 |
| 102 | L | 0.0157 | 0.0226 | 0 | 0.0069 |
| 103 | L | 0.0279 | 0.0376 | 0 | 0.0097 |
| 104 | L | 0.0144 | 0.0212 | 0 | 0.0068 |
| 105 | Q | 0.009 | 0.0128 | 0 | 0.0038 |
| 106 | F | 0.0179 | 0.0274 | 0 | 0.0095 |
| 107 | L | 0.0173 | 0.0279 | 0 | 0.0106 |
| 108 | Y | 0.0304 | 0.0455 | 0 | 0.0151 |
| 109 | S | 0.0245 | 0.0405 | 0 | 0.016 |
| 110 | G | 0.0405 | 0.066 | 0 | 0.0255 |
| 111 | Q | 0.0789 | 0.1292 | 0 | 0.0503 |
| 112 | V | 0.1117 | 0.1732 | 0 | 0.0615 |
| 113 | S | 0.1805 | 0.2602 | 0 | 0.0797 |
| 114 | I | 0.2432 | 0.3263 | 0 | 0.0831 |
| 115 | V | 0.1921 | 0.3939 | 1 | 0.2018 |
| 116 | P | 0.1844 | 0.3806 | 1 | 0.1962 |
| 117 | Q | 0.2786 | 0.4781 | 1 | 0.1995 |
| 118 | K | 0.3491 | 0.5473 | 1 | 0.1982 |
| 119 | H | 0.4333 | 0.6293 | 1 | 0.196 |
| 120 | E | 0.3053 | 0.6293 | 1 | 0.324 |
| 121 | P | 0.2255 | 0.5374 | 1 | 0.3119 |
| 122 | R | 0.2167 | 0.5331 | 1 | 0.3164 |
| 123 | P | 0.2786 | 0.5941 | 1 | 0.3155 |
| 124 | N | 0.2748 | 0.6035 | 1 | 0.3287 |
| 125 | S | 0.2333 | 0.6789 | 1 | 0.4456 |
| 126 | G | 0.3005 | 0.7342 | 1 | 0.4337 |
| 127 | E | 0.2531 | 0.6906 | 1 | 0.4375 |
| 128 | R | 0.2255 | 0.665 | 1 | 0.4395 |
| 129 | G | 0.1766 | 0.6174 | 1 | 0.4408 |
| 130 | S | 0.1958 | 0.6293 | 1 | 0.4335 |
| 131 | W | 0.1322 | 0.5514 | 1 | 0.4192 |
| 132 | H | 0.0991 | 0.5043 | 1 | 0.4052 |
| 133 | T | 0.066 | 0.4458 | 1 | 0.3798 |
| 134 | H | 0.0567 | 0.4245 | 1 | 0.3678 |
| 135 | S | 0.0567 | 0.4203 | 1 | 0.3636 |
| 136 | S | 0.0789 | 0.3535 | 1 | 0.2746 |
| 137 | A | 0.0643 | 0.3263 | 1 | 0.262 |
| 138 | A | 0.0478 | 0.282 | 1 | 0.2342 |
| 139 | V | 0.0478 | 0.2786 | 1 | 0.2308 |
| 140 | D | 0.0464 | 0.2748 | 0 | 0.2284 |
| 141 | L | 0.0587 | 0.1921 | 0 | 0.1334 |
| 142 | A | 0.0542 | 0.1878 | 0 | 0.1336 |
| 143 | L | 0.0554 | 0.1805 | 0 | 0.1251 |
| 144 | D | 0.0363 | 0.1292 | 0 | 0.0929 |
| 145 | T | 0.0405 | 0.1349 | 0 | 0.0944 |
| 146 | L | 0.0858 | 0.1349 | 0 | 0.0491 |
| 147 | A | 0.0464 | 0.0789 | 0 | 0.0325 |
| 148 | A | 0.0441 | 0.0701 | 0 | 0.026 |
| 149 | S | 0.0286 | 0.0464 | 0 | 0.0178 |
| 150 | R | 0.0231 | 0.0376 | 0 | 0.0145 |
| 151 | Y | 0.0218 | 0.0336 | 0 | 0.0118 |
| 152 | F | 0.0336 | 0.0502 | 0 | 0.0166 |
| 153 | G | 0.0387 | 0.0542 | 0 | 0.0155 |
| 154 | V | 0.0587 | 0.0813 | 0 | 0.0226 |
| 155 | E | 0.0316 | 0.0441 | 0 | 0.0125 |
| 156 | Q | 0.0259 | 0.0336 | 0 | 0.0077 |
| 157 | L | 0.0425 | 0.0542 | 0 | 0.0117 |
| 158 | A | 0.0414 | 0.0502 | 0 | 0.0088 |
| 159 | L | 0.0316 | 0.0387 | 0 | 0.0071 |
| 160 | L | 0.035 | 0.0414 | 0 | 0.0064 |
| 161 | T | 0.0363 | 0.0425 | 0 | 0.0062 |
| 162 | Q | 0.049 | 0.0567 | 0 | 0.0077 |
| 163 | K | 0.0909 | 0.1041 | 0 | 0.0132 |
| 164 | Q | 0.0526 | 0.0567 | 0 | 0.0041 |
| 165 | L | 0.0858 | 0.0935 | 0 | 0.0077 |
| 166 | A | 0.0813 | 0.0884 | 0 | 0.0071 |
| 167 | S | 0.0514 | 0.0554 | 0 | 0.004 |
| 168 | M | 0.0789 | 0.0858 | 0 | 0.0069 |
| 169 | V | 0.1018 | 0.106 | 0 | 0.0042 |
| 170 | E | 0.115 | 0.124 | 0 | 0.009 |
| 171 | K | 0.1266 | 0.1322 | 0 | 0.0056 |
| 172 | A | 0.0813 | 0.0858 | 0 | 0.0045 |
| 173 | S | 0.0677 | 0.0744 | 0 | 0.0067 |
| 174 | I | 0.0744 | 0.0789 | 0 | 0.0045 |
| 175 | E | 0.0744 | 0.0813 | 0 | 0.0069 |
| 176 | D | 0.1266 | 0.1349 | 0 | 0.0083 |
| 177 | V | 0.1532 | 0.1667 | 0 | 0.0135 |
| 178 | M | 0.1532 | 0.1667 | 0 | 0.0135 |
| 179 | K | 0.1602 | 0.1732 | 0 | 0.013 |
| 180 | V | 0.2209 | 0.2483 | 0 | 0.0274 |
| 181 | L | 0.2167 | 0.2432 | 0 | 0.0265 |
| 182 | I | 0.1532 | 0.1732 | 0 | 0.02 |
| 183 | A | 0.1178 | 0.1322 | 0 | 0.0144 |
| 184 | S | 0.1117 | 0.1292 | 0 | 0.0175 |
| 185 | R | 0.1766 | 0.1958 | 0 | 0.0192 |
| 186 | K | 0.1266 | 0.1958 | 0 | 0.0692 |
| 187 | Q | 0.1266 | 0.1921 | 0 | 0.0655 |
| 188 | D | 0.1844 | 0.27 | 0 | 0.0856 |
| 189 | M | 0.138 | 0.2167 | 0 | 0.0787 |
| 190 | H | 0.0991 | 0.1635 | 0 | 0.0644 |
| 191 | Q | 0.124 | 0.1958 | 0 | 0.0718 |
| 192 | L | 0.1844 | 0.27 | 0 | 0.0856 |
| 193 | W | 0.2602 | 0.3535 | 0 | 0.0933 |
| 194 | T | 0.2963 | 0.3847 | 0 | 0.0884 |
| 195 | T | 0.2041 | 0.2963 | 0 | 0.0922 |
| 196 | S | 0.2333 | 0.3263 | 0 | 0.093 |
| 197 | S | 0.2657 | 0.3578 | 0 | 0.0921 |
| 198 | H | 0.27 | 0.3578 | 0 | 0.0878 |
| 199 | L | 0.1958 | 0.282 | 0 | 0.0862 |
| 200 | V | 0.1635 | 0.2432 | 0 | 0.0797 |
| 201 | A | 0.138 | 0.2122 | 0 | 0.0742 |
| 202 | K | 0.138 | 0.208 | 0 | 0.07 |
| 203 | S | 0.1958 | 0.282 | 0 | 0.0862 |
| 204 | G | 0.1998 | 0.2913 | 0 | 0.0915 |
| 205 | L | 0.2255 | 0.3184 | 0 | 0.0929 |
| 206 | P | 0.1566 | 0.2385 | 0 | 0.0819 |
| 207 | P | 0.2041 | 0.2333 | 0 | 0.0292 |
| 208 | E | 0.1532 | 0.1805 | 0 | 0.0273 |
| 209 | I | 0.115 | 0.1322 | 0 | 0.0172 |
| 210 | L | 0.1732 | 0.1998 | 0 | 0.0266 |
| 211 | A | 0.2432 | 0.27 | 0 | 0.0268 |
| 212 | K | 0.1921 | 0.2122 | 0 | 0.0201 |
| 213 | H | 0.1921 | 0.2167 | 0 | 0.0246 |
| 214 | L | 0.1921 | 0.2167 | 0 | 0.0246 |
| 215 | P | 0.1292 | 0.1456 | 0 | 0.0164 |
| 216 | I | 0.1998 | 0.2209 | 0 | 0.0211 |
| 217 | D | 0.1178 | 0.1292 | 0 | 0.0114 |
| 218 | V | 0.1018 | 0.1117 | 0 | 0.0099 |
| 219 | V | 0.1041 | 0.115 | 0 | 0.0109 |
| 220 | T | 0.1602 | 0.1766 | 0 | 0.0164 |
| 221 | K | 0.1566 | 0.1698 | 0 | 0.0132 |
| 222 | I | 0.1635 | 0.1732 | 0 | 0.0097 |
| 223 | E | 0.1566 | 0.1667 | 0 | 0.0101 |
| 224 | E | 0.1602 | 0.1667 | 0 | 0.0065 |
| 225 | L | 0.2432 | 0.2483 | 0 | 0.0051 |
| 226 | R | 0.1566 | 0.1602 | 0 | 0.0036 |
| 227 | L | 0.2122 | 0.2209 | 0 | 0.0087 |
| 228 | K | 0.2531 | 0.2558 | 0 | 0.0027 |
| 229 | S | 0.3184 | 0.3225 | 0 | 0.0041 |
| 230 | S | 0.3806 | 0.3806 | 0 | 0 |
| 231 | I | 0.3847 | 0.3847 | 0 | 0 |
| 232 | A | 0.3806 | 0.3806 | 0 | 0 |
| 233 | R | 0.4781 | 0.4781 | 0 | 0 |
| 234 | R | 0.4749 | 0.4749 | 0 | 0 |
| 235 | S | 0.3992 | 0.3992 | 0 | 0 |
| 236 | L | 0.4825 | 0.4825 | 0 | 0 |
| 237 | M | 0.4282 | 0.4282 | 0 | 0 |
| 238 | P | 0.4879 | 0.4879 | 0 | 0 |
| 239 | H | 0.5008 | 0.5008 | 0 | 0 |
| 240 | N | 0.4879 | 0.4879 | 0 | 0 |
| 241 | H | 0.4037 | 0.4037 | 0 | 0 |
| 242 | H | 0.4781 | 0.4781 | 0 | 0 |
| 243 | H | 0.4967 | 0.4967 | 0 | 0 |
| 244 | D | 0.5173 | 0.5173 | 0 | 0 |
| 245 | L | 0.5253 | 0.5253 | 0 | 0 |
| 246 | S | 0.442 | 0.442 | 0 | 0 |
| 247 | V | 0.5084 | 0.5084 | 0 | 0 |
| 248 | A | 0.5229 | 0.5229 | 0 | 0 |
| 249 | Q | 0.46 | 0.46 | 0 | 0 |
| 250 | D | 0.4458 | 0.4458 | 0 | 0 |
| 251 | L | 0.4333 | 0.4333 | 0 | 0 |
| 252 | E | 0.4037 | 0.4037 | 0 | 0 |
| 253 | D | 0.3225 | 0.3225 | 0 | 0 |
| 254 | Q | 0.3096 | 0.3096 | 0 | 0 |
| 255 | K | 0.3184 | 0.3184 | 0 | 0 |
| 256 | I | 0.3992 | 0.3992 | 0 | 0 |
| 257 | R | 0.3939 | 0.3939 | 0 | 0 |
| 258 | R | 0.3806 | 0.3806 | 0 | 0 |
| 259 | M | 0.4116 | 0.4116 | 0 | 0 |
| 260 | R | 0.3146 | 0.3146 | 0 | 0 |
| 261 | R | 0.2602 | 0.2602 | 0 | 0 |
| 262 | A | 0.3311 | 0.3359 | 0 | 0.0048 |
| 263 | L | 0.2558 | 0.2602 | 0 | 0.0044 |
| 264 | D | 0.2385 | 0.2432 | 0 | 0.0047 |
| 265 | S | 0.1667 | 0.1732 | 0 | 0.0065 |
| 266 | S | 0.1416 | 0.1495 | 0 | 0.0079 |
| 267 | D | 0.208 | 0.2167 | 0 | 0.0087 |
| 268 | V | 0.2122 | 0.2255 | 0 | 0.0133 |
| 269 | E | 0.2167 | 0.2292 | 0 | 0.0125 |
| 270 | L | 0.1635 | 0.1732 | 0 | 0.0097 |
| 271 | V | 0.1698 | 0.1844 | 0 | 0.0146 |
| 272 | K | 0.1117 | 0.124 | 0 | 0.0123 |
| 273 | L | 0.1292 | 0.1456 | 0 | 0.0164 |
| 274 | M | 0.1998 | 0.2209 | 0 | 0.0211 |
| 275 | V | 0.1998 | 0.2209 | 0 | 0.0211 |
| 276 | M | 0.124 | 0.138 | 0 | 0.014 |
| 277 | G | 0.0965 | 0.1088 | 0 | 0.0123 |
| 278 | E | 0.0514 | 0.0621 | 0 | 0.0107 |
| 279 | G | 0.0771 | 0.0965 | 0 | 0.0194 |
| 280 | L | 0.0425 | 0.0526 | 0 | 0.0101 |
| 281 | N | 0.0554 | 0.0701 | 0 | 0.0147 |
| 282 | L | 0.0526 | 0.066 | 0 | 0.0134 |
| 283 | D | 0.0607 | 0.0771 | 0 | 0.0164 |
| 284 | E | 0.1018 | 0.1205 | 0 | 0.0187 |
| 285 | S | 0.0723 | 0.1349 | 0 | 0.0626 |
| 286 | L | 0.106 | 0.1805 | 0 | 0.0745 |
| 287 | A | 0.1205 | 0.2041 | 0 | 0.0836 |
| 288 | L | 0.1349 | 0.2209 | 0 | 0.086 |
| 289 | H | 0.0935 | 0.1667 | 0 | 0.0732 |
| 290 | Y | 0.0621 | 0.1178 | 0 | 0.0557 |
| 291 | A | 0.106 | 0.1766 | 0 | 0.0706 |
| 292 | V | 0.0832 | 0.1495 | 0 | 0.0663 |
| 293 | E | 0.0935 | 0.1635 | 0 | 0.07 |
| 294 | S | 0.0542 | 0.1041 | 0 | 0.0499 |
| 295 | S | 0.0567 | 0.1088 | 0 | 0.0521 |
| 296 | S | 0.0336 | 0.066 | 0 | 0.0324 |
| 297 | R | 0.0587 | 0.115 | 0 | 0.0563 |
| 298 | E | 0.0607 | 0.1178 | 0 | 0.0571 |
| 299 | V | 0.0858 | 0.1566 | 0 | 0.0708 |
| 300 | V | 0.0991 | 0.1667 | 0 | 0.0676 |
| 301 | K | 0.1041 | 0.1766 | 0 | 0.0725 |
| 302 | A | 0.1292 | 0.2122 | 0 | 0.083 |
| 303 | L | 0.115 | 0.1921 | 0 | 0.0771 |
| 304 | L | 0.1178 | 0.1921 | 0 | 0.0743 |
| 305 | E | 0.0909 | 0.1602 | 0 | 0.0693 |
| 306 | L | 0.1349 | 0.1602 | 0 | 0.0253 |
| 307 | G | 0.1495 | 0.1732 | 0 | 0.0237 |
| 308 | A | 0.1266 | 0.1495 | 0 | 0.0229 |
| 309 | A | 0.1088 | 0.1322 | 0 | 0.0234 |
| 310 | D | 0.1532 | 0.1766 | 0 | 0.0234 |
| 311 | V | 0.2041 | 0.2333 | 0 | 0.0292 |
| 312 | N | 0.2292 | 0.2657 | 0 | 0.0365 |
| 313 | Y | 0.1878 | 0.2167 | 0 | 0.0289 |
| 314 | P | 0.27 | 0.2963 | 0 | 0.0263 |
| 315 | A | 0.2558 | 0.282 | 0 | 0.0262 |
| 316 | G | 0.2122 | 0.2385 | 0 | 0.0263 |
| 317 | P | 0.2602 | 0.282 | 0 | 0.0218 |
| 318 | A | 0.282 | 0.3053 | 0 | 0.0233 |
| 319 | G | 0.2748 | 0.2963 | 0 | 0.0215 |
| 320 | K | 0.2558 | 0.2748 | 0 | 0.019 |
| 321 | T | 0.2432 | 0.2602 | 0 | 0.017 |
| 322 | P | 0.3359 | 0.3491 | 0 | 0.0132 |
| 323 | L | 0.3491 | 0.363 | 0 | 0.0139 |
| 324 | H | 0.3992 | 0.4116 | 0 | 0.0124 |
| 325 | I | 0.3184 | 0.3311 | 0 | 0.0127 |
| 326 | A | 0.3311 | 0.3399 | 0 | 0.0088 |
| 327 | A | 0.2786 | 0.2865 | 0 | 0.0079 |
| 328 | E | 0.1921 | 0.1998 | 0 | 0.0077 |
| 329 | M | 0.1667 | 0.1667 | 0 | 0 |
| 330 | V | 0.1766 | 0.1766 | 0 | 0 |
| 331 | S | 0.1766 | 0.1766 | 0 | 0 |
| 332 | P | 0.1732 | 0.1732 | 0 | 0 |
| 333 | D | 0.1292 | 0.1292 | 0 | 0 |
| 334 | M | 0.1921 | 0.1921 | 0 | 0 |
| 335 | V | 0.2122 | 0.2122 | 0 | 0 |
| 336 | A | 0.2963 | 0.2963 | 0 | 0 |
| 337 | V | 0.2531 | 0.2531 | 0 | 0 |
| 338 | L | 0.282 | 0.282 | 0 | 0 |
| 339 | L | 0.2602 | 0.2602 | 0 | 0 |
| 340 | D | 0.2255 | 0.2255 | 0 | 0 |
| 341 | H | 0.282 | 0.282 | 0 | 0 |
| 342 | H | 0.282 | 0.282 | 0 | 0 |
| 343 | A | 0.1766 | 0.1766 | 0 | 0 |
| 344 | D | 0.1635 | 0.1635 | 0 | 0 |
| 345 | P | 0.2255 | 0.2255 | 0 | 0 |
| 346 | N | 0.1998 | 0.1998 | 0 | 0 |
| 347 | V | 0.2292 | 0.2292 | 0 | 0 |
| 348 | R | 0.2041 | 0.2041 | 0 | 0 |
| 349 | T | 0.1878 | 0.1878 | 0 | 0 |
| 350 | V | 0.2531 | 0.2531 | 0 | 0 |
| 351 | G | 0.2385 | 0.2432 | 0 | 0.0047 |
| 352 | G | 0.1566 | 0.1602 | 0 | 0.0036 |
| 353 | I | 0.1566 | 0.1602 | 0 | 0.0036 |
| 354 | T | 0.1878 | 0.1921 | 0 | 0.0043 |
| 355 | P | 0.1878 | 0.1921 | 0 | 0.0043 |
| 356 | L | 0.0991 | 0.1041 | 0 | 0.005 |
| 357 | D | 0.0607 | 0.0643 | 0 | 0.0036 |
| 358 | I | 0.0542 | 0.0587 | 0 | 0.0045 |
| 359 | L | 0.0542 | 0.0567 | 0 | 0.0025 |
| 360 | R | 0.0554 | 0.0607 | 0 | 0.0053 |
| 361 | T | 0.0701 | 0.0789 | 0 | 0.0088 |
| 362 | L | 0.0414 | 0.0464 | 0 | 0.005 |
| 363 | T | 0.0502 | 0.0554 | 0 | 0.0052 |
| 364 | S | 0.0884 | 0.1018 | 0 | 0.0134 |
| 365 | D | 0.049 | 0.0554 | 0 | 0.0064 |
| 366 | F | 0.0414 | 0.0478 | 0 | 0.0064 |
| 367 | L | 0.066 | 0.0813 | 0 | 0.0153 |
| 368 | F | 0.035 | 0.0425 | 0 | 0.0075 |
| 369 | K | 0.0643 | 0.0789 | 0 | 0.0146 |
| 370 | G | 0.1292 | 0.1532 | 0 | 0.024 |
| 371 | A | 0.1292 | 0.1532 | 0 | 0.024 |
| 372 | V | 0.1292 | 0.1532 | 0 | 0.024 |
| 373 | P | 0.1349 | 0.1635 | 0 | 0.0286 |
| 374 | G | 0.138 | 0.1635 | 0 | 0.0255 |
| 375 | L | 0.0909 | 0.1088 | 0 | 0.0179 |
| 376 | T | 0.0587 | 0.1088 | 0 | 0.0501 |
| 377 | H | 0.0677 | 0.124 | 0 | 0.0563 |
| 378 | I | 0.115 | 0.1878 | 0 | 0.0728 |
| 379 | E | 0.1349 | 0.2167 | 0 | 0.0818 |
| 380 | P | 0.1041 | 0.1732 | 0 | 0.0691 |
| 381 | N | 0.1088 | 0.1844 | 0 | 0.0756 |
| 382 | K | 0.1349 | 0.2167 | 0 | 0.0818 |
| 383 | L | 0.1805 | 0.2748 | 0 | 0.0943 |
| 384 | R | 0.1349 | 0.2167 | 0 | 0.0818 |
| 385 | L | 0.1205 | 0.1958 | 0 | 0.0753 |
| 386 | S | 0.1602 | 0.2432 | 0 | 0.083 |
| 387 | L | 0.1088 | 0.1805 | 0 | 0.0717 |
| 388 | E | 0.1178 | 0.1844 | 0 | 0.0666 |
| 389 | L | 0.1921 | 0.2786 | 0 | 0.0865 |
| 390 | V | 0.1921 | 0.2786 | 0 | 0.0865 |
| 391 | Q | 0.1732 | 0.2558 | 0 | 0.0826 |
| 392 | S | 0.1732 | 0.2558 | 0 | 0.0826 |
| 393 | A | 0.1766 | 0.2602 | 0 | 0.0836 |
| 394 | A | 0.2558 | 0.3456 | 0 | 0.0898 |
| 395 | M | 0.2602 | 0.3491 | 0 | 0.0889 |
| 396 | V | 0.3311 | 0.4245 | 0 | 0.0934 |
| 397 | I | 0.4037 | 0.4245 | 0 | 0.0208 |
| 398 | S | 0.4879 | 0.5126 | 0 | 0.0247 |
| 399 | R | 0.4781 | 0.5084 | 0 | 0.0303 |
| 400 | E | 0.5412 | 0.5667 | 0 | 0.0255 |
| 401 | E | 0.6035 | 0.6334 | 0 | 0.0299 |
| 402 | G | 0.5941 | 0.6219 | 0 | 0.0278 |
| 403 | N | 0.5941 | 0.6219 | 0 | 0.0278 |
| 404 | N | 0.6255 | 0.6442 | 0 | 0.0187 |
| 405 | S | 0.6442 | 0.665 | 0 | 0.0208 |
| 406 | N | 0.6219 | 0.6412 | 0 | 0.0193 |
| 407 | N | 0.6174 | 0.6374 | 0 | 0.02 |
| 408 | Q | 0.6984 | 0.7192 | 0 | 0.0208 |
| 409 | N | 0.6984 | 0.7192 | 0 | 0.0208 |
| 410 | N | 0.6827 | 0.6984 | 0 | 0.0157 |
| 411 | D | 0.6756 | 0.6906 | 0 | 0.015 |
| 412 | N | 0.6789 | 0.6944 | 0 | 0.0155 |
| 413 | N | 0.687 | 0.7034 | 0 | 0.0164 |
| 414 | T | 0.6906 | 0.7079 | 0 | 0.0173 |
| 415 | G | 0.6906 | 0.7034 | 0 | 0.0128 |
| 416 | I | 0.6906 | 0.7034 | 0 | 0.0128 |
| 417 | Y | 0.6827 | 0.6944 | 0 | 0.0117 |
| 418 | P | 0.687 | 0.6944 | 0 | 0.0074 |
| 419 | H | 0.6789 | 0.6827 | 0 | 0.0038 |
| 420 | M | 0.6756 | 0.6756 | 0 | 0 |
| 421 | N | 0.6681 | 0.6715 | 0 | 0.0034 |
| 422 | E | 0.6604 | 0.6604 | 0 | 0 |
| 423 | E | 0.6604 | 0.6604 | 0 | 0 |
| 424 | H | 0.6604 | 0.6604 | 0 | 0 |
| 425 | N | 0.6604 | 0.6604 | 0 | 0 |
| 426 | S | 0.6293 | 0.6293 | 0 | 0 |
| 427 | G | 0.6789 | 0.6789 | 0 | 0 |
| 428 | S | 0.7275 | 0.7275 | 0 | 0 |
| 429 | S | 0.7111 | 0.7111 | 0 | 0 |
| 430 | G | 0.6715 | 0.6715 | 0 | 0 |
| 431 | G | 0.6681 | 0.6681 | 0 | 0 |
| 432 | S | 0.6219 | 0.6219 | 0 | 0 |
| 433 | N | 0.5846 | 0.5846 | 0 | 0 |
| 434 | N | 0.5762 | 0.5762 | 0 | 0 |
| 435 | N | 0.5374 | 0.5374 | 0 | 0 |
| 436 | L | 0.5412 | 0.5412 | 0 | 0 |
| 437 | D | 0.5253 | 0.5253 | 0 | 0 |
| 438 | S | 0.5331 | 0.5331 | 0 | 0 |
| 439 | R | 0.5296 | 0.5296 | 0 | 0 |
| 440 | L | 0.5253 | 0.5253 | 0 | 0 |
| 441 | V | 0.5623 | 0.5623 | 0 | 0 |
| 442 | Y | 0.5514 | 0.5514 | 0 | 0 |
| 443 | L | 0.5514 | 0.5514 | 0 | 0 |
| 444 | N | 0.5802 | 0.5802 | 0 | 0 |
| 445 | L | 0.5802 | 0.5802 | 0 | 0 |
| 446 | G | 0.59 | 0.59 | 0 | 0 |
| 447 | A | 0.6334 | 0.6334 | 0 | 0 |
| 448 | G | 0.6474 | 0.6474 | 0 | 0 |
| 449 | T | 0.6474 | 0.6474 | 0 | 0 |
| 450 | G | 0.6412 | 0.6412 | 0 | 0 |
| 451 | Q | 0.6756 | 0.6756 | 0 | 0 |
| 452 | M | 0.7079 | 0.7079 | 0 | 0 |
| 453 | G | 0.7573 | 0.7573 | 0 | 0 |
| 454 | P | 0.7951 | 0.7951 | 0 | 0 |
| 455 | G | 0.8242 | 0.8242 | 0 | 0 |
| 456 | R | 0.8626 | 0.8626 | 0 | 0 |
| 457 | D | 0.8713 | 0.8713 | 0 | 0 |
| 458 | Q | 0.8857 | 0.8857 | 0 | 0 |
| 459 | G | 0.8677 | 0.8677 | 0 | 0 |
| 460 | D | 0.8746 | 0.8746 | 0 | 0 |
| 461 | D | 0.8886 | 0.8886 | 0 | 0 |
| 462 | H | 0.8746 | 0.8746 | 0 | 0 |
| 463 | N | 0.8945 | 0.8945 | 0 | 0 |
| 464 | S | 0.904 | 0.904 | 0 | 0 |
| 465 | Q | 0.8886 | 0.8886 | 0 | 0 |
| 466 | R | 0.8945 | 0.8945 | 0 | 0 |
| 467 | E | 0.9141 | 0.9141 | 0 | 0 |
| 468 | G | 0.9103 | 0.9103 | 0 | 0 |
| 469 | M | 0.9103 | 0.9103 | 0 | 0 |
| 470 | S | 0.9162 | 0.9162 | 0 | 0 |
| 471 | R | 0.9141 | 0.9141 | 0 | 0 |
| 472 | H | 0.904 | 0.904 | 0 | 0 |
| 473 | H | 0.8595 | 0.8595 | 0 | 0 |
| 474 | H | 0.865 | 0.865 | 0 | 0 |
| 475 | H | 0.865 | 0.865 | 0 | 0 |
| 476 | H | 0.8521 | 0.8521 | 0 | 0 |
| 477 | Q | 0.8556 | 0.8556 | 0 | 0 |
| 478 | D | 0.8713 | 0.8713 | 0 | 0 |
| 479 | P | 0.8781 | 0.8781 | 0 | 0 |
| 480 | S | 0.9103 | 0.9103 | 0 | 0 |
| 481 | T | 0.8457 | 0.8457 | 0 | 0 |
| 482 | M | 0.865 | 0.865 | 0 | 0 |
| 483 | Y | 0.8857 | 0.8857 | 0 | 0 |
| 484 | H | 0.904 | 0.904 | 0 | 0 |
| 485 | H | 0.918 | 0.918 | 0 | 0 |
| 486 | H | 0.9316 | 0.9316 | 0 | 0 |
| 487 | H | 0.9466 | 0.9466 | 0 | 0 |
| 488 | Q | 0.9466 | 0.9466 | 0 | 0 |
| 489 | H | 0.9653 | 0.9653 | 0 | 0 |
| 490 | H | 0.9684 | 0.9684 | 0 | 0 |
| 491 | F | 0.976 | 0.976 | 0 | 0 |
| >AtNPR6 | | | | | |
| (AT3G57130) | | | | | |
| 1 | M | 0.8521 | 0.8521 | 0 | 0 |
| 2 | S | 0.7817 | 0.7817 | 0 | 0 |
| 3 | N | 0.7388 | 0.7388 | 0 | 0 |
| 4 | T | 0.6255 | 0.6255 | 0 | 0 |
| 5 | F | 0.59 | 0.59 | 0 | 0 |
| 6 | E | 0.4651 | 0.4651 | 0 | 0 |
| 7 | E | 0.3399 | 0.3399 | 0 | 0 |
| 8 | S | 0.3005 | 0.3053 | 0 | 0.0048 |
| 9 | L | 0.1805 | 0.1844 | 0 | 0.0039 |
| 10 | K | 0.0935 | 0.0965 | 0 | 0.003 |
| 11 | S | 0.0405 | 0.0425 | 0 | 0.002 |
| 12 | M | 0.0526 | 0.0554 | 0 | 0.0028 |
| 13 | S | 0.0542 | 0.0587 | 0 | 0.0045 |
| 14 | L | 0.0567 | 0.0621 | 0 | 0.0054 |
| 15 | D | 0.0464 | 0.0514 | 0 | 0.005 |
| 16 | Y | 0.0441 | 0.0502 | 0 | 0.0061 |
| 17 | L | 0.0387 | 0.0425 | 0 | 0.0038 |
| 18 | N | 0.0336 | 0.0387 | 0 | 0.0051 |
| 19 | L | 0.02 | 0.0231 | 0 | 0.0031 |
| 20 | L | 0.0363 | 0.0414 | 0 | 0.0051 |
| 21 | I | 0.0167 | 0.02 | 0 | 0.0033 |
| 22 | N | 0.0167 | 0.02 | 0 | 0.0033 |
| 23 | G | 0.0118 | 0.0141 | 0 | 0.0023 |
| 24 | Q | 0.0128 | 0.0157 | 0 | 0.0029 |
| 25 | A | 0.0252 | 0.0304 | 0 | 0.0052 |
| 26 | F | 0.0226 | 0.0279 | 0 | 0.0053 |
| 27 | S | 0.02 | 0.0268 | 0 | 0.0068 |
| 28 | D | 0.02 | 0.0274 | 0 | 0.0074 |
| 29 | V | 0.0173 | 0.0252 | 0 | 0.0079 |
| 30 | T | 0.0252 | 0.0336 | 0 | 0.0084 |
| 31 | F | 0.0387 | 0.0526 | 0 | 0.0139 |
| 32 | S | 0.0621 | 0.0909 | 0 | 0.0288 |
| 33 | V | 0.0327 | 0.0858 | 0 | 0.0531 |
| 34 | E | 0.02 | 0.0526 | 0 | 0.0326 |
| 35 | G | 0.0099 | 0.0308 | 0 | 0.0209 |
| 36 | R | 0.0109 | 0.0327 | 0 | 0.0218 |
| 37 | L | 0.0212 | 0.0554 | 0 | 0.0342 |
| 38 | V | 0.0212 | 0.0567 | 0 | 0.0355 |
| 39 | H | 0.02 | 0.0554 | 0 | 0.0354 |
| 40 | A | 0.0226 | 0.0621 | 0 | 0.0395 |
| 41 | H | 0.0124 | 0.0395 | 0 | 0.0271 |
| 42 | R | 0.0173 | 0.0514 | 0 | 0.0341 |
| 43 | S | 0.0173 | 0.0526 | 0 | 0.0353 |
| 44 | I | 0.0327 | 0.0935 | 0 | 0.0608 |
| 45 | L | 0.0173 | 0.0542 | 0 | 0.0369 |
| 46 | A | 0.0097 | 0.0327 | 0 | 0.023 |
| 47 | A | 0.0055 | 0.0336 | 0 | 0.0281 |
| 48 | R | 0.0116 | 0.066 | 0 | 0.0544 |
| 49 | S | 0.0212 | 0.106 | 0 | 0.0848 |
| 50 | L | 0.0259 | 0.1178 | 0 | 0.0919 |
| 51 | F | 0.0395 | 0.1667 | 0 | 0.1272 |
| 52 | F | 0.0441 | 0.1667 | 0 | 0.1226 |
| 53 | R | 0.049 | 0.1878 | 0 | 0.1388 |
| 54 | K | 0.106 | 0.2167 | 0 | 0.1107 |
| 55 | F | 0.1566 | 0.2913 | 0 | 0.1347 |
| 56 | F | 0.1921 | 0.3359 | 0 | 0.1438 |
| 57 | S | 0.2292 | 0.3717 | 0 | 0.1425 |
| 58 | E | 0.282 | 0.4245 | 0 | 0.1425 |
| 59 | S | 0.2531 | 0.3939 | 0 | 0.1408 |
| 60 | D | 0.2531 | 0.3992 | 0 | 0.1461 |
| 61 | P | 0.3359 | 0.4781 | 0 | 0.1422 |
| 62 | S | 0.4116 | 0.5514 | 0 | 0.1398 |
| 63 | Q | 0.4918 | 0.6255 | 0 | 0.1337 |
| 64 | P | 0.4879 | 0.6219 | 0 | 0.134 |
| 65 | G | 0.4879 | 0.6219 | 0 | 0.134 |
| 66 | A | 0.5253 | 0.6557 | 0 | 0.1304 |
| 67 | E | 0.5992 | 0.7232 | 0 | 0.124 |
| 68 | P | 0.6557 | 0.6906 | 0 | 0.0349 |
| 69 | A | 0.6079 | 0.6474 | 0 | 0.0395 |
| 70 | N | 0.5711 | 0.6174 | 0 | 0.0463 |
| 71 | Q | 0.5008 | 0.5374 | 0 | 0.0366 |
| 72 | T | 0.4703 | 0.5126 | 0 | 0.0423 |
| 73 | G | 0.4749 | 0.5173 | 0 | 0.0424 |
| 74 | S | 0.3939 | 0.4333 | 0 | 0.0394 |
| 75 | G | 0.2786 | 0.3184 | 0 | 0.0398 |
| 76 | A | 0.3096 | 0.3399 | 0 | 0.0303 |
| 77 | R | 0.27 | 0.2963 | 0 | 0.0263 |
| 78 | A | 0.2602 | 0.2865 | 0 | 0.0263 |
| 79 | A | 0.2333 | 0.2602 | 0 | 0.0269 |
| 80 | A | 0.1998 | 0.2255 | 0 | 0.0257 |
| 81 | V | 0.2041 | 0.2209 | 0 | 0.0168 |
| 82 | G | 0.1266 | 0.138 | 0 | 0.0114 |
| 83 | G | 0.1349 | 0.1495 | 0 | 0.0146 |
| 84 | V | 0.0935 | 0.1018 | 0 | 0.0083 |
| 85 | I | 0.0542 | 0.0607 | 0 | 0.0065 |
| 86 | P | 0.0336 | 0.0387 | 0 | 0.0051 |
| 87 | V | 0.0279 | 0.0304 | 0 | 0.0025 |
| 88 | N | 0.016 | 0.0179 | 0 | 0.0019 |
| 89 | S | 0.0121 | 0.0137 | 0 | 0.0016 |
| 90 | V | 0.0167 | 0.02 | 0 | 0.0033 |
| 91 | G | 0.0102 | 0.0124 | 0 | 0.0022 |
| 92 | Y | 0.0094 | 0.0118 | 0 | 0.0024 |
| 93 | E | 0.0059 | 0.0069 | 0 | 0.001 |
| 94 | V | 0.0059 | 0.0069 | 0 | 0.001 |
| 95 | F | 0.0083 | 0.0099 | 0 | 0.0016 |
| 96 | L | 0.016 | 0.0231 | 0 | 0.0071 |
| 97 | L | 0.0084 | 0.0118 | 0 | 0.0034 |
| 98 | L | 0.0144 | 0.0212 | 0 | 0.0068 |
| 99 | L | 0.0081 | 0.0102 | 0 | 0.0021 |
| 100 | Q | 0.0055 | 0.0075 | 0 | 0.002 |
| 101 | F | 0.0097 | 0.0144 | 0 | 0.0047 |
| 102 | L | 0.0086 | 0.0141 | 0 | 0.0055 |
| 103 | Y | 0.015 | 0.0259 | 0 | 0.0109 |
| 104 | S | 0.0118 | 0.0226 | 0 | 0.0108 |
| 105 | G | 0.0212 | 0.0387 | 0 | 0.0175 |
| 106 | Q | 0.0464 | 0.0813 | 0 | 0.0349 |
| 107 | V | 0.0701 | 0.1205 | 0 | 0.0504 |
| 108 | S | 0.1088 | 0.1732 | 0 | 0.0644 |
| 109 | I | 0.1635 | 0.2432 | 0 | 0.0797 |
| 110 | V | 0.1292 | 0.3184 | 1 | 0.1892 |
| 111 | P | 0.1205 | 0.3053 | 1 | 0.1848 |
| 112 | H | 0.2041 | 0.4078 | 1 | 0.2037 |
| 113 | K | 0.282 | 0.4879 | 1 | 0.2059 |
| 114 | H | 0.3668 | 0.5667 | 1 | 0.1999 |
| 115 | E | 0.2385 | 0.5667 | 1 | 0.3282 |
| 116 | P | 0.1667 | 0.4749 | 1 | 0.3082 |
| 117 | R | 0.1566 | 0.4703 | 1 | 0.3137 |
| 118 | S | 0.2041 | 0.5253 | 1 | 0.3212 |
| 119 | N | 0.1958 | 0.5331 | 1 | 0.3373 |
| 120 | S | 0.1635 | 0.6124 | 1 | 0.4489 |
| 121 | G | 0.2122 | 0.665 | 1 | 0.4528 |
| 122 | D | 0.1667 | 0.6174 | 1 | 0.4507 |
| 123 | R | 0.1602 | 0.5992 | 1 | 0.439 |
| 124 | G | 0.1205 | 0.5473 | 1 | 0.4268 |
| 125 | S | 0.1349 | 0.5514 | 1 | 0.4165 |
| 126 | W | 0.0789 | 0.4703 | 1 | 0.3914 |
| 127 | H | 0.066 | 0.4379 | 1 | 0.3719 |
| 128 | T | 0.0441 | 0.3717 | 1 | 0.3276 |
| 129 | H | 0.0441 | 0.3762 | 1 | 0.3321 |
| 130 | S | 0.0274 | 0.3005 | 1 | 0.2731 |
| 131 | T | 0.0376 | 0.2333 | 1 | 0.1957 |
| 132 | A | 0.0308 | 0.2041 | 0 | 0.1733 |
| 133 | A | 0.0245 | 0.1732 | 0 | 0.1487 |
| 134 | V | 0.0179 | 0.1495 | 0 | 0.1316 |
| 135 | D | 0.0179 | 0.1456 | 0 | 0.1277 |
| 136 | L | 0.0245 | 0.0909 | 0 | 0.0664 |
| 137 | S | 0.0218 | 0.0909 | 0 | 0.0691 |
| 138 | L | 0.0231 | 0.0858 | 0 | 0.0627 |
| 139 | D | 0.0137 | 0.0554 | 0 | 0.0417 |
| 140 | I | 0.0157 | 0.0587 | 0 | 0.043 |
| 141 | L | 0.0376 | 0.0607 | 0 | 0.0231 |
| 142 | A | 0.0194 | 0.0327 | 0 | 0.0133 |
| 143 | A | 0.0179 | 0.0304 | 0 | 0.0125 |
| 144 | A | 0.0109 | 0.0179 | 0 | 0.007 |
| 145 | R | 0.0084 | 0.0137 | 0 | 0.0053 |
| 146 | Y | 0.0083 | 0.0131 | 0 | 0.0048 |
| 147 | F | 0.0141 | 0.0231 | 0 | 0.009 |
| 148 | G | 0.0131 | 0.0218 | 0 | 0.0087 |
| 149 | V | 0.0259 | 0.0376 | 0 | 0.0117 |
| 150 | E | 0.0124 | 0.0182 | 0 | 0.0058 |
| 151 | Q | 0.0212 | 0.0304 | 0 | 0.0092 |
| 152 | L | 0.0363 | 0.049 | 0 | 0.0127 |
| 153 | A | 0.0336 | 0.0455 | 0 | 0.0119 |
| 154 | L | 0.0268 | 0.0327 | 0 | 0.0059 |
| 155 | L | 0.0363 | 0.0441 | 0 | 0.0078 |
| 156 | T | 0.0387 | 0.0455 | 0 | 0.0068 |
| 157 | Q | 0.0514 | 0.0607 | 0 | 0.0093 |
| 158 | K | 0.1018 | 0.1117 | 0 | 0.0099 |
| 159 | H | 0.0554 | 0.0621 | 0 | 0.0067 |
| 160 | L | 0.0935 | 0.1018 | 0 | 0.0083 |
| 161 | T | 0.0858 | 0.0935 | 0 | 0.0077 |
| 162 | S | 0.0542 | 0.0567 | 0 | 0.0025 |
| 163 | M | 0.0832 | 0.0909 | 0 | 0.0077 |
| 164 | V | 0.106 | 0.1117 | 0 | 0.0057 |
| 165 | E | 0.124 | 0.1292 | 0 | 0.0052 |
| 166 | K | 0.1349 | 0.1416 | 0 | 0.0067 |
| 167 | A | 0.0858 | 0.0909 | 0 | 0.0051 |
| 168 | S | 0.0701 | 0.0771 | 0 | 0.007 |
| 169 | I | 0.0771 | 0.0813 | 0 | 0.0042 |
| 170 | E | 0.0744 | 0.0789 | 0 | 0.0045 |
| 171 | D | 0.1266 | 0.1349 | 0 | 0.0083 |
| 172 | V | 0.1292 | 0.1416 | 0 | 0.0124 |
| 173 | M | 0.1266 | 0.138 | 0 | 0.0114 |
| 174 | K | 0.1322 | 0.1456 | 0 | 0.0134 |
| 175 | V | 0.1921 | 0.2122 | 0 | 0.0201 |
| 176 | L | 0.1805 | 0.2041 | 0 | 0.0236 |
| 177 | I | 0.1205 | 0.138 | 0 | 0.0175 |
| 178 | A | 0.0858 | 0.1018 | 0 | 0.016 |
| 179 | S | 0.0832 | 0.0991 | 0 | 0.0159 |
| 180 | R | 0.1416 | 0.1602 | 0 | 0.0186 |
| 181 | K | 0.0991 | 0.1602 | 0 | 0.0611 |
| 182 | Q | 0.0991 | 0.1566 | 0 | 0.0575 |
| 183 | D | 0.0909 | 0.1456 | 0 | 0.0547 |
| 184 | M | 0.0621 | 0.106 | 0 | 0.0439 |
| 185 | H | 0.0376 | 0.0621 | 0 | 0.0245 |
| 186 | Q | 0.0455 | 0.0789 | 0 | 0.0334 |
| 187 | L | 0.0744 | 0.124 | 0 | 0.0496 |
| 188 | W | 0.1266 | 0.1921 | 0 | 0.0655 |
| 189 | T | 0.1495 | 0.2209 | 0 | 0.0714 |
| 190 | T | 0.0858 | 0.1416 | 0 | 0.0558 |
| 191 | S | 0.1041 | 0.1635 | 0 | 0.0594 |
| 192 | S | 0.106 | 0.1698 | 0 | 0.0638 |
| 193 | Y | 0.1117 | 0.1732 | 0 | 0.0615 |
| 194 | L | 0.066 | 0.115 | 0 | 0.049 |
| 195 | I | 0.0502 | 0.0884 | 0 | 0.0382 |
| 196 | A | 0.0414 | 0.0701 | 0 | 0.0287 |
| 197 | K | 0.0414 | 0.0701 | 0 | 0.0287 |
| 198 | S | 0.066 | 0.1117 | 0 | 0.0457 |
| 199 | G | 0.066 | 0.115 | 0 | 0.049 |
| 200 | L | 0.0813 | 0.1349 | 0 | 0.0536 |
| 201 | P | 0.0464 | 0.0813 | 0 | 0.0349 |
| 202 | Q | 0.0701 | 0.0832 | 0 | 0.0131 |
| 203 | E | 0.0395 | 0.0478 | 0 | 0.0083 |
| 204 | I | 0.0478 | 0.0554 | 0 | 0.0076 |
| 205 | L | 0.0677 | 0.0813 | 0 | 0.0136 |
| 206 | A | 0.1205 | 0.1416 | 0 | 0.0211 |
| 207 | K | 0.0832 | 0.1018 | 0 | 0.0186 |
| 208 | H | 0.0884 | 0.106 | 0 | 0.0176 |
| 209 | L | 0.0935 | 0.1088 | 0 | 0.0153 |
| 210 | P | 0.0526 | 0.0621 | 0 | 0.0095 |
| 211 | I | 0.0991 | 0.1117 | 0 | 0.0126 |
| 212 | E | 0.049 | 0.0567 | 0 | 0.0077 |
| 213 | L | 0.049 | 0.0554 | 0 | 0.0064 |
| 214 | V | 0.0502 | 0.0554 | 0 | 0.0052 |
| 215 | A | 0.0884 | 0.1018 | 0 | 0.0134 |
| 216 | K | 0.124 | 0.1349 | 0 | 0.0109 |
| 217 | I | 0.1732 | 0.1844 | 0 | 0.0112 |
| 218 | E | 0.1117 | 0.1205 | 0 | 0.0088 |
| 219 | E | 0.115 | 0.1205 | 0 | 0.0055 |
| 220 | L | 0.1921 | 0.1958 | 0 | 0.0037 |
| 221 | R | 0.1117 | 0.1178 | 0 | 0.0061 |
| 222 | L | 0.1667 | 0.1732 | 0 | 0.0065 |
| 223 | K | 0.1878 | 0.1958 | 0 | 0.008 |
| 224 | S | 0.2748 | 0.2786 | 0 | 0.0038 |
| 225 | S | 0.3399 | 0.3399 | 0 | 0 |
| 226 | M | 0.3717 | 0.3717 | 0 | 0 |
| 227 | P | 0.3668 | 0.3668 | 0 | 0 |
| 228 | L | 0.3847 | 0.3847 | 0 | 0 |
| 229 | R | 0.3762 | 0.3762 | 0 | 0 |
| 230 | S | 0.3762 | 0.3762 | 0 | 0 |
| 231 | L | 0.46 | 0.46 | 0 | 0 |
| 232 | M | 0.3847 | 0.3847 | 0 | 0 |
| 233 | P | 0.4651 | 0.4651 | 0 | 0 |
| 234 | H | 0.3847 | 0.3847 | 0 | 0 |
| 235 | H | 0.3762 | 0.3762 | 0 | 0 |
| 236 | H | 0.3668 | 0.3668 | 0 | 0 |
| 237 | D | 0.4037 | 0.4037 | 0 | 0 |
| 238 | L | 0.3762 | 0.3762 | 0 | 0 |
| 239 | T | 0.3847 | 0.3847 | 0 | 0 |
| 240 | S | 0.3806 | 0.3806 | 0 | 0 |
| 241 | T | 0.3717 | 0.3717 | 0 | 0 |
| 242 | L | 0.4245 | 0.4245 | 0 | 0 |
| 243 | D | 0.4333 | 0.4333 | 0 | 0 |
| 244 | L | 0.3885 | 0.3885 | 0 | 0 |
| 245 | E | 0.3668 | 0.3668 | 0 | 0 |
| 246 | D | 0.2865 | 0.2865 | 0 | 0 |
| 247 | Q | 0.2865 | 0.2865 | 0 | 0 |
| 248 | K | 0.2913 | 0.2913 | 0 | 0 |
| 249 | I | 0.3762 | 0.3762 | 0 | 0 |
| 250 | R | 0.3717 | 0.3717 | 0 | 0 |
| 251 | R | 0.3005 | 0.3005 | 0 | 0 |
| 252 | M | 0.3096 | 0.3096 | 0 | 0 |
| 253 | R | 0.3005 | 0.3005 | 0 | 0 |
| 254 | R | 0.2385 | 0.2432 | 0 | 0.0047 |
| 255 | A | 0.3096 | 0.3146 | 0 | 0.005 |
| 256 | L | 0.2255 | 0.2292 | 0 | 0.0037 |
| 257 | D | 0.2041 | 0.2122 | 0 | 0.0081 |
| 258 | S | 0.138 | 0.1456 | 0 | 0.0076 |
| 259 | S | 0.1178 | 0.124 | 0 | 0.0062 |
| 260 | D | 0.1732 | 0.1805 | 0 | 0.0073 |
| 261 | V | 0.1766 | 0.1844 | 0 | 0.0078 |
| 262 | E | 0.1766 | 0.1921 | 0 | 0.0155 |
| 263 | L | 0.1266 | 0.138 | 0 | 0.0114 |
| 264 | V | 0.1322 | 0.1456 | 0 | 0.0134 |
| 265 | K | 0.0813 | 0.0935 | 0 | 0.0122 |
| 266 | L | 0.0991 | 0.1117 | 0 | 0.0126 |
| 267 | M | 0.1635 | 0.1805 | 0 | 0.017 |
| 268 | V | 0.1635 | 0.1766 | 0 | 0.0131 |
| 269 | M | 0.0935 | 0.106 | 0 | 0.0125 |
| 270 | G | 0.0677 | 0.0813 | 0 | 0.0136 |
| 271 | E | 0.0395 | 0.0464 | 0 | 0.0069 |
| 272 | G | 0.0327 | 0.0395 | 0 | 0.0068 |
| 273 | L | 0.0167 | 0.0212 | 0 | 0.0045 |
| 274 | N | 0.0245 | 0.0297 | 0 | 0.0052 |
| 275 | L | 0.0226 | 0.0274 | 0 | 0.0048 |
| 276 | D | 0.0268 | 0.0316 | 0 | 0.0048 |
| 277 | E | 0.0455 | 0.0526 | 0 | 0.0071 |
| 278 | S | 0.035 | 0.0621 | 0 | 0.0271 |
| 279 | L | 0.0514 | 0.0935 | 0 | 0.0421 |
| 280 | A | 0.0607 | 0.1088 | 0 | 0.0481 |
| 281 | L | 0.0701 | 0.1205 | 0 | 0.0504 |
| 282 | I | 0.0464 | 0.0813 | 0 | 0.0349 |
| 283 | Y | 0.0304 | 0.0526 | 0 | 0.0222 |
| 284 | A | 0.0514 | 0.0909 | 0 | 0.0395 |
| 285 | V | 0.0405 | 0.0701 | 0 | 0.0296 |
| 286 | E | 0.0441 | 0.0771 | 0 | 0.033 |
| 287 | N | 0.0252 | 0.0455 | 0 | 0.0203 |
| 288 | S | 0.0259 | 0.0464 | 0 | 0.0205 |
| 289 | S | 0.0131 | 0.0274 | 0 | 0.0143 |
| 290 | R | 0.0268 | 0.0478 | 0 | 0.021 |
| 291 | E | 0.0274 | 0.049 | 0 | 0.0216 |
| 292 | V | 0.0387 | 0.0677 | 0 | 0.029 |
| 293 | V | 0.0744 | 0.1292 | 0 | 0.0548 |
| 294 | K | 0.0789 | 0.1349 | 0 | 0.056 |
| 295 | A | 0.0991 | 0.1667 | 0 | 0.0676 |
| 296 | L | 0.0884 | 0.1495 | 0 | 0.0611 |
| 297 | L | 0.0909 | 0.1532 | 0 | 0.0623 |
| 298 | E | 0.0677 | 0.124 | 0 | 0.0563 |
| 299 | L | 0.1018 | 0.1205 | 0 | 0.0187 |
| 300 | G | 0.115 | 0.1349 | 0 | 0.0199 |
| 301 | A | 0.115 | 0.1349 | 0 | 0.0199 |
| 302 | A | 0.0991 | 0.1205 | 0 | 0.0214 |
| 303 | D | 0.138 | 0.1667 | 0 | 0.0287 |
| 304 | V | 0.1921 | 0.2255 | 0 | 0.0334 |
| 305 | N | 0.1732 | 0.1998 | 0 | 0.0266 |
| 306 | Y | 0.1456 | 0.1667 | 0 | 0.0211 |
| 307 | P | 0.2167 | 0.2483 | 0 | 0.0316 |
| 308 | A | 0.2122 | 0.2385 | 0 | 0.0263 |
| 309 | G | 0.1732 | 0.1921 | 0 | 0.0189 |
| 310 | P | 0.2209 | 0.2432 | 0 | 0.0223 |
| 311 | T | 0.2483 | 0.27 | 0 | 0.0217 |
| 312 | G | 0.2432 | 0.2602 | 0 | 0.017 |
| 313 | K | 0.2292 | 0.2432 | 0 | 0.014 |
| 314 | T | 0.2167 | 0.2333 | 0 | 0.0166 |
| 315 | A | 0.3184 | 0.3311 | 0 | 0.0127 |
| 316 | L | 0.3311 | 0.3456 | 0 | 0.0145 |
| 317 | H | 0.3847 | 0.3939 | 0 | 0.0092 |
| 318 | I | 0.3053 | 0.3146 | 0 | 0.0093 |
| 319 | A | 0.3184 | 0.3263 | 0 | 0.0079 |
| 320 | A | 0.2657 | 0.27 | 0 | 0.0043 |
| 321 | E | 0.1805 | 0.1844 | 0 | 0.0039 |
| 322 | M | 0.1266 | 0.1292 | 0 | 0.0026 |
| 323 | V | 0.138 | 0.138 | 0 | 0 |
| 324 | S | 0.138 | 0.138 | 0 | 0 |
| 325 | P | 0.1349 | 0.1349 | 0 | 0 |
| 326 | D | 0.1349 | 0.1349 | 0 | 0 |
| 327 | M | 0.1998 | 0.1998 | 0 | 0 |
| 328 | V | 0.2209 | 0.2209 | 0 | 0 |
| 329 | A | 0.3096 | 0.3096 | 0 | 0 |
| 330 | V | 0.2602 | 0.2602 | 0 | 0 |
| 331 | L | 0.3096 | 0.3096 | 0 | 0 |
| 332 | L | 0.282 | 0.282 | 0 | 0 |
| 333 | D | 0.2483 | 0.2483 | 0 | 0 |
| 334 | H | 0.3096 | 0.3096 | 0 | 0 |
| 335 | H | 0.3096 | 0.3096 | 0 | 0 |
| 336 | A | 0.1958 | 0.1958 | 0 | 0 |
| 337 | D | 0.1805 | 0.1805 | 0 | 0 |
| 338 | P | 0.2483 | 0.2483 | 0 | 0 |
| 339 | N | 0.2209 | 0.2209 | 0 | 0 |
| 340 | V | 0.2558 | 0.2558 | 0 | 0 |
| 341 | Q | 0.2255 | 0.2255 | 0 | 0 |
| 342 | T | 0.208 | 0.208 | 0 | 0 |
| 343 | V | 0.2748 | 0.2748 | 0 | 0 |
| 344 | D | 0.2602 | 0.2602 | 0 | 0 |
| 345 | G | 0.1698 | 0.1732 | 0 | 0.0034 |
| 346 | I | 0.1698 | 0.1732 | 0 | 0.0034 |
| 347 | T | 0.2041 | 0.208 | 0 | 0.0039 |
| 348 | P | 0.2041 | 0.208 | 0 | 0.0039 |
| 349 | L | 0.1117 | 0.115 | 0 | 0.0033 |
| 350 | D | 0.0677 | 0.0723 | 0 | 0.0046 |
| 351 | I | 0.0621 | 0.066 | 0 | 0.0039 |
| 352 | L | 0.0542 | 0.0567 | 0 | 0.0025 |
| 353 | R | 0.0554 | 0.0607 | 0 | 0.0053 |
| 354 | T | 0.0723 | 0.0813 | 0 | 0.009 |
| 355 | L | 0.035 | 0.0387 | 0 | 0.0037 |
| 356 | T | 0.0425 | 0.0478 | 0 | 0.0053 |
| 357 | S | 0.0771 | 0.0858 | 0 | 0.0087 |
| 358 | D | 0.0414 | 0.0478 | 0 | 0.0064 |
| 359 | F | 0.0327 | 0.0395 | 0 | 0.0068 |
| 360 | L | 0.0542 | 0.0643 | 0 | 0.0101 |
| 361 | F | 0.0279 | 0.0327 | 0 | 0.0048 |
| 362 | K | 0.0514 | 0.0621 | 0 | 0.0107 |
| 363 | G | 0.1088 | 0.1292 | 0 | 0.0204 |
| 364 | A | 0.1088 | 0.1292 | 0 | 0.0204 |
| 365 | I | 0.1088 | 0.1292 | 0 | 0.0204 |
| 366 | P | 0.115 | 0.1349 | 0 | 0.0199 |
| 367 | G | 0.115 | 0.1349 | 0 | 0.0199 |
| 368 | L | 0.0701 | 0.0858 | 0 | 0.0157 |
| 369 | T | 0.0455 | 0.0832 | 0 | 0.0377 |
| 370 | H | 0.049 | 0.0909 | 0 | 0.0419 |
| 371 | I | 0.0884 | 0.1495 | 0 | 0.0611 |
| 372 | E | 0.0935 | 0.1566 | 0 | 0.0631 |
| 373 | P | 0.0643 | 0.115 | 0 | 0.0507 |
| 374 | N | 0.0677 | 0.124 | 0 | 0.0563 |
| 375 | K | 0.0909 | 0.1495 | 0 | 0.0586 |
| 376 | L | 0.138 | 0.2167 | 0 | 0.0787 |
| 377 | R | 0.0991 | 0.1667 | 0 | 0.0676 |
| 378 | L | 0.066 | 0.1178 | 0 | 0.0518 |
| 379 | S | 0.0935 | 0.1602 | 0 | 0.0667 |
| 380 | L | 0.0587 | 0.106 | 0 | 0.0473 |
| 381 | E | 0.0643 | 0.1117 | 0 | 0.0474 |
| 382 | L | 0.1205 | 0.1878 | 0 | 0.0673 |
| 383 | V | 0.1178 | 0.1844 | 0 | 0.0666 |
| 384 | Q | 0.1018 | 0.1635 | 0 | 0.0617 |
| 385 | S | 0.1041 | 0.1667 | 0 | 0.0626 |
| 386 | A | 0.106 | 0.1698 | 0 | 0.0638 |
| 387 | A | 0.1698 | 0.2531 | 0 | 0.0833 |
| 388 | L | 0.1732 | 0.2602 | 0 | 0.087 |
| 389 | V | 0.2483 | 0.3359 | 0 | 0.0876 |
| 390 | I | 0.3146 | 0.3399 | 0 | 0.0253 |
| 391 | S | 0.3992 | 0.4245 | 0 | 0.0253 |
| 392 | R | 0.3885 | 0.4149 | 0 | 0.0264 |
| 393 | E | 0.4703 | 0.4967 | 0 | 0.0264 |
| 394 | E | 0.5296 | 0.5549 | 0 | 0.0253 |
| 395 | G | 0.4918 | 0.5173 | 0 | 0.0255 |
| 396 | N | 0.4149 | 0.442 | 0 | 0.0271 |
| 397 | N | 0.3806 | 0.4078 | 0 | 0.0272 |
| 398 | N | 0.4333 | 0.4556 | 0 | 0.0223 |
| 399 | S | 0.5008 | 0.5173 | 0 | 0.0165 |
| 400 | N | 0.5229 | 0.5374 | 0 | 0.0145 |
| 401 | D | 0.59 | 0.6079 | 0 | 0.0179 |
| 402 | N | 0.59 | 0.6079 | 0 | 0.0179 |
| 403 | N | 0.5941 | 0.6174 | 0 | 0.0233 |
| 404 | T | 0.5846 | 0.6035 | 0 | 0.0189 |
| 405 | M | 0.5762 | 0.5941 | 0 | 0.0179 |
| 406 | I | 0.5802 | 0.5941 | 0 | 0.0139 |
| 407 | Y | 0.5802 | 0.59 | 0 | 0.0098 |
| 408 | P | 0.5802 | 0.5941 | 0 | 0.0139 |
| 409 | R | 0.5846 | 0.5941 | 0 | 0.0095 |
| 410 | M | 0.5374 | 0.5473 | 0 | 0.0099 |
| 411 | K | 0.5374 | 0.5473 | 0 | 0.0099 |
| 412 | D | 0.5374 | 0.5412 | 0 | 0.0038 |
| 413 | E | 0.5374 | 0.5412 | 0 | 0.0038 |
| 414 | H | 0.4918 | 0.4918 | 0 | 0 |
| 415 | T | 0.46 | 0.46 | 0 | 0 |
| 416 | S | 0.4333 | 0.4333 | 0 | 0 |
| 417 | G | 0.4513 | 0.4513 | 0 | 0 |
| 418 | S | 0.5229 | 0.5229 | 0 | 0 |
| 419 | S | 0.4379 | 0.4379 | 0 | 0 |
| 420 | L | 0.4379 | 0.4379 | 0 | 0 |
| 421 | D | 0.4458 | 0.4458 | 0 | 0 |
| 422 | S | 0.4458 | 0.4458 | 0 | 0 |
| 423 | R | 0.4458 | 0.4458 | 0 | 0 |
| 424 | L | 0.4458 | 0.4458 | 0 | 0 |
| 425 | V | 0.4458 | 0.4458 | 0 | 0 |
| 426 | Y | 0.3939 | 0.3939 | 0 | 0 |
| 427 | L | 0.3939 | 0.3939 | 0 | 0 |
| 428 | N | 0.3939 | 0.3939 | 0 | 0 |
| 429 | L | 0.3847 | 0.3847 | 0 | 0 |
| 430 | G | 0.3847 | 0.3847 | 0 | 0 |
| 431 | A | 0.4333 | 0.4333 | 0 | 0 |
| 432 | T | 0.4379 | 0.4379 | 0 | 0 |
| 433 | N | 0.4703 | 0.4703 | 0 | 0 |
| 434 | R | 0.4749 | 0.4749 | 0 | 0 |
| 435 | D | 0.5331 | 0.5331 | 0 | 0 |
| 436 | I | 0.5623 | 0.5623 | 0 | 0 |
| 437 | G | 0.5846 | 0.5846 | 0 | 0 |
| 438 | D | 0.6412 | 0.6412 | 0 | 0 |
| 439 | D | 0.5762 | 0.5762 | 0 | 0 |
| 440 | N | 0.6374 | 0.6374 | 0 | 0 |
| 441 | S | 0.6334 | 0.6334 | 0 | 0 |
| 442 | N | 0.6474 | 0.6474 | 0 | 0 |
| 443 | Q | 0.6516 | 0.6516 | 0 | 0 |
| 444 | R | 0.6557 | 0.6557 | 0 | 0 |
| 445 | E | 0.6412 | 0.6412 | 0 | 0 |
| 446 | G | 0.665 | 0.665 | 0 | 0 |
| 447 | M | 0.7232 | 0.7232 | 0 | 0 |
| 448 | N | 0.7192 | 0.7192 | 0 | 0 |
| 449 | L | 0.7034 | 0.7034 | 0 | 0 |
| 450 | H | 0.6557 | 0.6557 | 0 | 0 |
| 451 | H | 0.6604 | 0.6604 | 0 | 0 |
| 452 | H | 0.6557 | 0.6557 | 0 | 0 |
| 453 | H | 0.6557 | 0.6557 | 0 | 0 |
| 454 | H | 0.6334 | 0.6334 | 0 | 0 |
| 455 | D | 0.6334 | 0.6334 | 0 | 0 |
| 456 | P | 0.6374 | 0.6374 | 0 | 0 |
| 457 | S | 0.5514 | 0.5514 | 0 | 0 |
| 458 | T | 0.6255 | 0.6255 | 0 | 0 |
| 459 | M | 0.6557 | 0.6557 | 0 | 0 |
| 460 | Y | 0.7458 | 0.7458 | 0 | 0 |
| 461 | H | 0.7772 | 0.7772 | 0 | 0 |
| 462 | H | 0.82 | 0.82 | 0 | 0 |
| 463 | H | 0.8488 | 0.8488 | 0 | 0 |
| 464 | H | 0.8746 | 0.8746 | 0 | 0 |
| 465 | H | 0.9009 | 0.9009 | 0 | 0 |
| 466 | H | 0.9299 | 0.9299 | 0 | 0 |
| 467 | F | 0.9369 | 0.9369 | 0 | 0 |

TABLE 4
Map of NPR1 Mutations. Related to FIGS. 2A-2I. List of gain/loss of function point mutations and
their alleles found in previously published forward and reverse genetic screens of AtNPR1.

| AA position | WT AA | Mutated AA | Mutant name | Reference |
|---|---|---|---|---|
| 1 | M | | | |
| 2 | D | | | |
| 3 | T | | | |
| 4 | T | | | |
| 5 | I | | | |
| 6 | D | | | |
| 7 | G | | | |
| 8 | F | | | |
| 9 | A | | | |
| 10 | D | | | |
| 11 | S | A/D | S11/15A/D | Spoel et al. 2009 |
| 12 | Y | | | |
| 13 | E | | | |
| 14 | I | | | |
| 15 | S | A/D | S11/15A/D | Spoel et al. 2009 |
| 16 | S | | | |
| 17 | T | | | |
| 18 | S | | | |
| 19 | F | | | |
| 20 | V | | | |
| 21 | A | | | |
| 22 | T | | | |
| 23 | D | | | |
| 24 | N | | | |
| 25 | T | | | |
| 26 | D | | | |
| 27 | S | | | |
| 28 | S | | | |
| 29 | I | | | |
| 30 | V | | | |
| 31 | Y | | | |
| 32 | L | | | |
| 33 | A | | | |
| 34 | A | | | |
| 35 | E | | | |
| 36 | Q | | | |
| 37 | V | | | |
| 38 | L | | | |
| 39 | T | | | |
| 40 | G | | | |
| 41 | P | | | |
| 42 | D | | | |
| 43 | V | | | |
| 44 | S | | | |
| 45 | A | | | |
| 46 | L | | | |
| 47 | Q | | | |
| 48 | L | | | |
| 49 | L | | | |
| 50 | S | | | |
| 51 | N | | | |
| 52 | S | | | |
| 53 | F | | | |
| 54 | E | | | |
| 55 | S | A/D | S55/59A/D | Saleh et al. 2015 |
| 56 | V | | | |
| 57 | F | | | |
| 58 | D | | | |
| 59 | S | A/D | S55/59A/D | Saleh et al. 2015 |
| 60 | P | | | |
| 61 | D | | | |
| 62 | D | | | |
| 63 | F | | | |
| 64 | Y | N | npr1-58 | Canet et al. 2010 |
| 65 | S | | | |
| 66 | D | | | |
| 67 | A | | | |
| 68 | K | | | |
| 69 | L | | | |
| 70 | V | | | |
| 71 | L | | | |
| 72 | S | | | |
| 73 | D | | | |
| 74 | G | | | |
| 75 | R | | | |
| 76 | E | | | |
| 77 | V | | | |
| 78 | S | | | |
| 79 | F | | | |
| 80 | H | Y/A | npr1-44, H80A | Canet et al. 2010, Rochon et al. 2006 |
| 81 | R | A | R81A | Rochon et al. 2006 |
| 82 | C | A | C82A | Mou et al. 2003, Rochon et al. 2006 |
| 83 | V | A | V83A | Rochon et al. 2006 |
| 84 | L | A | L84A | Rochon et al. 2006 |
| 85 | S | | | |
| 86 | A | | | |
| 87 | R | A | R87A | Rochon et al. 2006 |
| 88 | S | A | S88A | Rochon et al. 2006 |
| 89 | S | A | S89A | Rochon et al. 2006 |
| 90 | F | A | F90A | Rochon et al. 2006 |
| 91 | F | A | F91A | Rochon et al. 2006 |
| 92 | K | | | |
| 93 | S | | | |
| 94 | A | | | |
| 95 | L | | | |
| 96 | A | | | |
| 97 | A | | | |
| 98 | A | | | |
| 99 | K | | | |
| 100 | K | | | |
| 101 | E | | | |
| 102 | K | | | |
| 103 | D | | | |
| 104 | S | | | |
| 105 | N | | | |
| 106 | N | | | |
| 107 | T | | | |
| 108 | A | | | |
| 109 | A | | | |
| 110 | V | | | |
| 111 | K | | | |
| 112 | L | | | |
| 113 | E | | | |
| 114 | L | | | |
| 115 | K | | | |
| 116 | E | | | |
| 117 | I | | | |
| 118 | A | | | |
| 119 | K | | | |
| 120 | D | | | |
| 121 | Y | | | |
| 122 | E | | | |
| 123 | V | | | |
| 124 | G | | | |
| 125 | F | | | |
| 126 | D | | | |
| 127 | S | | | |
| 128 | V | | | |
| 129 | V | | | |
| 130 | T | | | |
| 131 | V | | | |
| 132 | L | | | |
| 133 | A | | | |
| 134 | Y | | | |
| 135 | V | | | |
| 136 | Y | | | |
| 137 | S | | | |
| 138 | S | | | |
| 139 | R | | | |
| 140 | V | | | |
| 141 | R | | | |
| 142 | P | | | |
| 143 | P | | | |
| 144 | P | | | |
| 145 | K | | | |
| 146 | G | | | |
| 147 | V | | | |
| 148 | S | | | |
| 149 | E | | | |
| 150 | C | Y/A | rdr1, npr1-2 | This paper, Cao et al. 1997 |
| 151 | A | | | |
| 152 | D | | | |
| 153 | E | | | |
| 154 | N | | | |
| 155 | C | Y/A | rdr1, npr1-35 | This paper, Canet et al. 2010 |
| 156 | C | A | rdr1, C156A | This paper, Tada et al. 2008 |
| 157 | H | | | |
| 158 | V | | | |
| 159 | A | | | |
| 160 | C | A | rdr1 | This paper |
| 161 | R | | | |
| 162 | P | frame shift | nim1-1 | Ryals et al. 1997 |
| 163 | A | | | |
| 164 | V | | | |
| 165 | D | | | |
| 166 | F | | | |
| 167 | M | | | |
| 168 | L | | | |
| 169 | E | | | |
| 170 | V | | | |
| 171 | L | frame shift | nim1-3 | Ryals et al. 1997 |
| 172 | Y | | | |
| 173 | L | | | |
| 174 | A | | | |
| 175 | F | | | |
| 176 | I | | | |
| 177 | F | | | |
| 178 | K | | | |
| 179 | I | | | |
| 180 | P | | | |
| 181 | E | | | |
| 182 | L | | | |
| 183 | I | | | |
| 184 | T | | | |
| 185 | L | | | |
| 186 | Y | | | |
| 187 | Q | | | |
| 188 | R | | | |
| 189 | H | | | |
| 190 | L | | | |
| 191 | L | | | |
| 192 | D | | | |
| 193 | V | | | |
| 194 | V | | | |
| 195 | D | | | |
| 196 | K | | | |
| 197 | V | | | |
| 198 | V | | | |
| 199 | I | | | |
| 200 | E | | | |
| 201 | D | | | |
| 202 | T | | | |
| 203 | L | | | |
| 204 | V | | | |
| 205 | I | | | |
| 206 | L | | | |
| 207 | K | | | |
| 208 | L | | | |
| 209 | A | | | |
| 210 | N | | | |
| 211 | I | | | |
| 212 | C | | | |
| 213 | G | | | |
| 214 | K | | | |
| 215 | A | | | |
| 216 | C | A | C216A | Mou et al. 2003 |
| 217 | M | | | |
| 218 | K | | | |
| 219 | L | | | |
| 220 | L | | | |
| 221 | D | | | |
| 222 | R | | | |
| 223 | C | | | |
| 224 | K | | | |
| 225 | E | | | |
| 226 | I | | | |
| 227 | I | | | |
| 228 | V | | | |
| 229 | K | | | |
| 230 | S | | | |
| 231 | N | | | |
| 232 | V | | | |
| 233 | D | | | |
| 234 | M | | | |
| 235 | V | | | |
| 236 | S | | | |
| 237 | L | | | |
| 238 | E | | | |
| 239 | K | | | |
| 240 | S | | | |
| 241 | L | | | |
| 242 | P | | | |
| 243 | E | | | |
| 244 | E | | | |
| 245 | L | | | |
| 246 | V | | | |
| 247 | K | | | |
| 248 | E | | | |
| 249 | I | | | |
| 250 | I | | | |
| 251 | D | | | |
| 252 | R | | | |
| 253 | R | | | |
| 254 | K | | | |
| 255 | E | | | |
| 256 | L | | | |
| 257 | G | | | |
| 258 | L | | | |
| 259 | E | | | |
| 260 | V | | | |
| 261 | P | | | |
| 262 | K | | | |
| 263 | V | | | |
| 264 | K | | | |
| 265 | K | | | |
| 266 | H | | | |
| 267 | V | | | |
| 268 | S | | | |
| 269 | N | | | |
| 270 | V | | | |
| 271 | H | | | |
| 272 | K | | | |
| 273 | A | | | |
| 274 | L | F | npr1-50 | Canet et al. 2010 |
| 275 | D | | | |
| 276 | S | | | |
| 277 | D | | | |
| 278 | D | | | |
| 279 | I | | | |
| 280 | E | | | |
| 281 | L | | | |
| 282 | V | | | |
| 283 | K | | | |
| 284 | L | | | |
| 285 | L | | | |
| 286 | L | | | |
| 287 | K | | | |
| 288 | E | K | npr1-41 | Canet et al. 2010 |
| 289 | D | | | |
| 290 | H | | | |
| 291 | T | | | |
| 292 | N | | | |
| 293 | L | | | |
| 294 | D | | | |
| 295 | D | | | |
| 296 | A | | | |
| 297 | C | | | |
| 298 | A | | | |
| 299 | L | | | |
| 300 | H | Y | nim1-2 | Ryals et al. 1997 |
| 301 | F | | | |
| 302 | A | | | |
| 303 | V | | | |
| 304 | A | | | |
| 305 | Y | | | |
| 306 | C | Y | npr1-40 | Canet et al. 2010 |
| 307 | N | | | |
| 308 | V | | | |
| 309 | K | | | |
| 310 | T | | | |
| 311 | A | | | |
| 312 | T | | | |
| 313 | D | | | |
| 314 | L | | | |
| 315 | L | | | |
| 316 | K | | | |
| 317 | L | | | |
| 318 | D | | | |
| 319 | L | | | |
| 320 | A | | | |
| 321 | D | | | |
| 322 | V | | | |
| 323 | N | | | |
| 324 | H | | | |
| 325 | R | | | |
| 326 | N | | | |
| 327 | P | | | |
| 328 | R | | | |
| 329 | G | | | |
| 330 | Y | | | |
| 331 | T | | | |
| 332 | V | | | |
| 333 | L | | | |
| 334 | H | Y | npr1-1 | Cao et al. 1997 |
| 335 | V | | | |
| 336 | A | | | |
| 337 | A | | | |
| 338 | M | | | |
| 339 | R | | | |
| 340 | K | | | |
| 341 | E | | | |
| 342 | P | S | npr1-5, npr1-60 | Shah et al. 1999, Canet et al. 2010 |
| 343 | Q | STOP | npr1-53 | Canet et al. 2010 |
| 344 | L | | | |
| 345 | I | A | sim3 | Saleh et al. 2015 |
| 346 | L | A | sim3 | Saleh et al. 2015 |
| 347 | S | | | |
| 348 | L | A | sim3 | Saleh et al. 2015 |
| 349 | L | | | |
| 350 | E | | | |
| 351 | K | | | |
| 352 | G | | | |
| 353 | A | | | |
| 354 | S | | | |
| 355 | A | | | |
| 356 | S | | | |
| 357 | E | | | |
| 358 | A | | | |
| 359 | T | | | |
| 360 | L | | | |
| 361 | E | | | |
| 362 | G | | | |
| 363 | R | | | |
| 364 | T | | | |
| 365 | A | | | |
| 366 | L | | | |
| 367 | M | | | |
| 368 | I | | | |
| 369 | A | | | |
| 370 | K | | | |
| 371 | Q | STOP | npr1-45 | Canet et al. 2010 |
| 372 | A | | | |
| 373 | T | A | T373A | Lee et al. 2015 |
| 374 | M | | | |
| 375 | A | | | |
| 376 | V | | | |
| 377 | E | | | |
| 378 | C | A | rdr2 | This paper |
| 379 | N | | | |
| 380 | N | | | |
| 381 | I | | | |
| 382 | P | | | |
| 383 | E | | | |
| 384 | Q | STOP | npr1-21 | Canet et al. 2010 |
| 385 | C | A | rdr2 | This paper |
| 386 | K | | | |
| 387 | H | | | |
| 388 | S | | | |
| 389 | L | | | |
| 390 | K | | | |
| 391 | G | | | |
| 392 | R | | | |
| 393 | L | | | |
| 394 | C | A | rdr2 | This paper |
| 395 | V | | | |
| 396 | E | | | |
| 397 | I | | | |
| 398 | L | | | |
| 399 | E | | | |
| 400 | Q | STOP | npr1-4 | Cao et al. 1997 |
| 401 | E | | | |
| 402 | D | | | |
| 403 | K | | | |
| 404 | R | | | |
| 405 | E | | | |
| 406 | Q | | | |
| 407 | I | | | |
| 408 | P | | | |
| 409 | R | | | |
| 410 | D | | | |
| 411 | V | | | |
| 412 | P | | | |
| 413 | P | | | |
| 414 | S | | | |
| 415 | F | | | |
| 416 | A | | | |
| 417 | V | | | |
| 418 | A | | | |
| 419 | A | | | |
| 420 | D | | | |
| 421 | E | | | |
| 422 | L | | | |
| 423 | K | | | |
| 424 | M | | | |
| 425 | T | | | |
| 426 | L | | | |
| 427 | L | | | |
| 428 | D | N | npr1-27 | Canet et al. 2010 |
| 429 | L | | | |
| 430 | E | | | |
| 431 | N | | | |
| 432 | R | K/Q | nim1-4, nim1-5, npr1-32, | Ryals et al. 1997, Canet et al. 2010, |
| | | | npr1-33, npr1-51, R432Q | Ding et al 2018 |
| 433 | V | | | |
| 434 | A | | | |
| 435 | L | | | |
| 436 | A | | | |
| 437 | Q | | | |
| 438 | R | | | |
| 439 | L | | | |
| 440 | F | | | |
| 441 | P | | | |
| 442 | T | | | |
| 443 | E | K | npr1-25 | Canet et al. 2010 |
| 444 | A | | | |
| 445 | Q | | | |
| 446 | A | | | |
| 447 | A | | | |
| 448 | M | | | |
| 449 | E | K | npr1-31, npr1-36 | Canet et al. 2010 |
| 450 | I | | | |
| 451 | A | V | npr1-30 | Canet et al. 2010 |
| 452 | E | | | |
| 453 | M | | | |
| 454 | K | | | |
| 455 | G | | | |
| 456 | T | | | |
| 457 | C | | | |
| 458 | E | | | |
| 459 | F | | | |
| 460 | I | | | |
| 461 | V | | | |
| 462 | T | | | |
| 463 | S | | | |
| 464 | L | | | |
| 465 | E | | | |
| 466 | P | | | |
| 467 | D | | | |
| 468 | R | | | |
| 469 | L | | | |
| 470 | T | | | |
| 471 | G | | | |
| 472 | T | | | |
| 473 | K | | | |
| 474 | R | | | |
| 475 | T | | | |
| 476 | S | | | |
| 477 | P | | | |
| 478 | G | | | |
| 479 | V | | | |
| 480 | K | | | |
| 481 | I | | | |
| 482 | A | | | |
| 483 | P | | | |
| 484 | F | | | |
| 485 | R | | | |
| 486 | I | | | |
| 487 | L | | | |
| 488 | E | | | |
| 489 | E | | | |
| 490 | H | | | |
| 491 | Q | STOP | npr1-3, npr1-37, npr1-49, | Cao et al. 1997, Ryals et al. 1997, |
| | | | npr1-55, nim1-6 | Canet et al. 2010 |
| 492 | S | | | |
| 493 | R | K | npr1-38 | Canet et al. 2010 |
| 494 | L | | | |
| 495 | K | | | |
| 496 | A | P | npr1-56 | Canet et al. 2010 |
| 497 | L | F | npr1-24, npr1-46, npr1-47 | Canet et al. 2010 |
| 498 | S | | | |
| 499 | K | | | |
| 500 | T | | | |
| 501 | V | M | npr1-20 | Canet et al. 2010 |
| 502 | E | | | |
| 503 | L | | | |
| 504 | G | E | npr1-42 | Canet et al. 2010 |
| 505 | K | | | |
| 506 | R | | | |
| 507 | F | S | F507S | Maier et al. 2011 |
| 508 | F | S | F508S | Maier et al. 2011 |
| 509 | P | | | |
| 510 | R | | | |
| 511 | C | A | rdr3 | This paper |
| 512 | S | L | npr1-39 | Canet et al. 2010 |
| 513 | A | | | |
| 514 | V | | | |
| 515 | L | F | npr1-48 | Canet et al. 2010 |
| 516 | D | | | |
| 517 | Q | | | |
| 518 | I | | | |
| 519 | M | | | |
| 520 | N | | | |
| 521 | C | A/S | rdr3, C521S | This paper, |
| | | | | Rochon et al. 2006, |
| | | | | Wu et al. 2012 |
| 522 | E | | | |
| 523 | D | | | |
| 524 | L | | | |
| 525 | T | | | |
| 526 | Q | STOP | npr1-34 | Canet et al. 2010 |
| 527 | L | | | |
| 528 | A | | | |
| 529 | C | A/S | rdr3, C529S | This paper, |
| | | | | Rochon et al. 2006, |
| | | | | Wu et al. 2012 |
| 530 | G | | | |
| 531 | E | | | |
| 532 | D | | | |
| 533 | D | | | |
| 534 | T | | | |
| 535 | A | | | |
| 536 | E | | | |
| 537 | K | | | |
| 538 | R | STOP | npr1-28 | Canet et al. 2010 |
| 539 | L | | | |
| 540 | Q | | | |
| 541 | K | Q | nls | Kinkema et al. 2000 |
| 542 | K | Q | nls | Kinkema et al. 2000 |
| 543 | Q | | | |
| 544 | R | K/G | npr1-22, nls | Kinkema et al. 2000, |
| | | | | Canet et al. 2010 |
| 545 | Y | | | |
| 546 | M | | | |
| 547 | E | | | |
| 548 | I | | | |
| 549 | Q | | | |
| 550 | E | | | |
| 551 | T | | | |
| 552 | L | | | |
| 553 | K | Q | nls | Kinkema et al. 2000 |
| 554 | K | Q | nls | Kinkema et al. 2000 |
| 555 | A | | | |
| 556 | F | | | |
| 557 | S | | | |
| 558 | E | | | |
| 559 | D | | | |
| 560 | N | | | |
| 561 | L | | | |
| 562 | E | | | |
| 563 | L | | | |
| 564 | G | | | |
| 565 | N | | | |
| 566 | S | | | |
| 567 | S | | | |
| 568 | L | | | |
| 569 | T | | | |
| 570 | D | | | |
| 571 | S | | | |
| 572 | T | | | |
| 573 | S | | | |
| 574 | S | | | |
| 575 | T | | | |
| 576 | S | | | |
| 577 | K | | | |
| 578 | S | | | |
| 579 | T | | | |
| 580 | G | | | |
| 581 | G | | | |
| 582 | K | | | |
| 583 | R | | | |
| 584 | S | | | |
| 585 | N | | | |
| 586 | R | | | |
| 587 | K | | | |
| 588 | L | | | |
| 589 | S | A | S589A | Lee et al. 2015 |
| 590 | H | | | |
| 591 | R | | | |
| 592 | R | | | |
| 593 | R | | | |

### REFERENCES

1) Canet J V et al. (2010). Structure-function analysis of npr1 alleles in Arabidopsis reveals a role for its paralogs in the perception of salicylic acid. Plant Cell Environ 33, 1911-1922.
2) Cao H et al. (1997). The Arabidopsis NPR1 gene that controls systemic acquired resistance encodes a novel protein containing ankyrin repeats. Cell 88, 57-63.
3) Ding Y et al. (2018). Opposite Roles of Salicylic Acid Receptors NPR1 and NPR3/NPR4 in Transcriptional Regulation of Plant Immunity. Cell 173, 1454-1467 e1415.
4) Kinkema M et al. (2000). “Nuclear localization of NPR1 is required for activation of PR gene expression.” The Plant cell 12, 2339-2350.
5) Lee H J et al. (2015). Systemic Immunity Requires SnRK2.8-Mediated Nuclear Import of NPR1 in Arabidopsis. The Plant cell 27, 3425-3438.
6) Maier F et al. (2011). NONEXPRESSOR OF PATHOGENESIS-RELATED PROTEINS1 (NPR1) and some NPR1-related proteins are sensitive to salicylic acid. Molecular plant pathology 12, 73-91.
7) Mou Z et al. (2003). Inducers of plant systemic acquired resistance regulate NPR1 function through redox changes. Cell 113, 935-944.
8) Rochon A et al. (2006). The coactivator function of Arabidopsis NPR1 requires the core of its BTB/POZ domain and the oxidation of C-terminal cysteines. The Plant cell 18, 3670-3685.
9) Ryals J et al. (1997). The Arabidopsis NIM1 protein shows homology to the mammalian transcription factor inhibitor I kappa B. The Plant cell 9, 425-439.
10) Saleh A et al. (2015). Posttranslational Modifications of the Master Transcriptional Regulator NPR1 Enable Dynamic but Tight Control of Plant Immune Responses. Cell Host Microbe 18, 169-182.
11) Shah J et al. (1999). The Arabidopsis ssi1 mutation restores pathogenesis-related gene expression in npr1 plants and renders defensin gene expression salicylic acid dependent. The Plant cell 11, 191-206.
12) Spoel S H et al. (2009) “Proteasome-mediated turnover of the transcription coactivator NPR1 plays dual roles in regulating plant immunity.” Cell 137, 860-872.
13) Tada Y et al. (2008). Plant immunity requires conformational changes of NPR1 via S-nitrosylation and thioredoxins. Science (New York, N.Y. 321, 952-956.
14) Wu Y et al. (2012). The Arabidopsis NPR1 protein is a receptor for the plant defense hormone salicylic acid. Cell reports 1, 639-647.

TABLE 5
Functional Categorization of sim3-GFP Interactome Proteins. Related to FTGS. 3A-3F.
SN Components - Part 1

| SINC | | | | | |
|---|---|---|---|---|---|
| Components | | | Normalized | | |
| Functional | | | Fold Change: | | |
| Groups | Gene ID | Symbol/Name | SA vs Mock | p-value | Description |
| D, E, F, G | AT4G05420 | DDB1A, damaged | 2.521654986 | 0.0002806 | Structurally similar to damaged DNA binding proteins. |
| | | DNA binding protein | | | DDB1a is part of a 350 KDa nuclear localized DET1 |
| | | 1A | | | protein complex. This complex may physically interact |
| | | | | | with histone tails and while bound to chromatin- |
| | | | | | repress transcription of genes involved in |
| | | | | | photomorphogenesis. DDB1a is shown to be RUB- |
| | | | | | modified. |
| D, E, F, G | AT5G50340 | ATP-dependent | 2.951968756 | 0.01 | DNA repair protein RadA-like protein |
| | | peptidase | | | |
| E, F, G | AT5G10450 | GRF6, G-box | 2.012355655 | 0.002 | member of the 14-3-3 gene family that is a lambda |
| | | regulating factor 6 | | | isoform (14-3-3λ). Interacts with APX3 (ascorbate |
| | | | | | peroxidase) and AKR2, suggesting a role in mediating |
| | | | | | oxidative metabolism in stress response. This protein |
| | | | | | was shown to colocalize and interact with SERK1 by |
| | | | | | which it is phosphorylated. This protein is also |
| | | | | | reported to interact with the phosphorylated form of |
| | | | | | the BZR1 transcription factor involved in |
| | | | | | brassinosteroid signaling and may affect the |
| | | | | | nucleocytoplasmic shuttling of BZR1. Interacts with |
| | | | | | JAZ10.4 which lacks the Jas motif. It is also |
| | | | | | phosphorylated by CRPK1 as part of the response to |
| | | | | | cold and translocates to the nucleus after |
| | | | | | phosphorylation. |
| E, F, G | AT5G44070 | PCS1, phytochelatin | 3.42876493 | 0.014 | Phytochelatin synthase gene confers tolerance to |
| | | synthase 1 (PCS1) | | | cadmium ions. Catalyzes phytochelatin (PC) synthesis |
| | | | | | from glutathione (GSH) in the presence of Cd2+, |
| | | | | | Zn2+, Cu2+ and Fe3+, but not by Co2+ or Ni2+. The |
| | | | | | mRNA is cell-to-cell mobile. |
| C, F, G | AT1G05010 | EFE, ethylene-forming | 2.159064852 | 0.00002881 | 1-aminocyclopropane-1-carboxylate oxidase |
| | | enzyme | | | |
| F, G | AT1G02930 | GSTF6, glutathione S- | 2.00932338 | 0.0008488 | glutathione transferase belonging to the phi class of |
| | | transferase 6 | | | GSTs. Naming convention according to Wagner et al. |
| | | | | | (2002). |
| F, G | AT1G04980 | PDIL2-2, PDI-like 2-2 | 2.195873033 | 0.042 | protein disulfide isomerase-like (PDIL) protein, a |
| | | | | | member of a multigene family within the thioredoxin |
| | | | | | (TRX) superfamily. Transcript levels for this gene are |
| | | | | | up-regulated in response to three different chemical |
| | | | | | inducers of ER stress (dithiothreitol, beta- |
| | | | | | mercaptoethanol, and tunicamycin). AtIRE1-2 does |
| | | | | | not appear to be required for this response, but the |
| | | | | | atbzip60 mutant has a diminished response. |
| F, G | AT1G54100 | ALDH7B4, aldehyde | 2.005991063 | 0.005 | Aldehyde dehydrogenase |
| | | dehydrogenase 7B4 | | | |
| F, G | AT1G63460 | GPX8, glutathione | 19.55048744 | 0.007 | GPX8 (glutathione peroxidase 8). Involved in the |
| | | peroxidase 8 | | | suppression of oxidative damages in nucleus and |
| | | | | | cytosol. The mRNA is cell-to-cell mobile. |
| F, G | AT1G76680 | OPR1, 12- | 4111.7295 | 0.00005161 | member of an alpha/beta barrel fold family of FMN- |
| | | oxophytodienoate | | | containing oxidoreductases. One of the closely related |
| | | reductase 1 | | | 12-oxophytodienoic acid reductases. This enzyme is |
| | | | | | not expected to participate in jasmonic acid |
| | | | | | biosynthesis because during in vitro assays, it shows |
| | | | | | very little activity with the naturally occurring OPDA |
| | | | | | isomer. Shows activity towards 2,4,6-trinitrotoluene. |
| | | | | | Expressed predominately in root. Up-regulated by |
| | | | | | senescence and jasmonic acid. Induced by salicylic |
| | | | | | acid. Independent of NPR1 for their induction by |
| | | | | | salicylic acid. Predicted to be a cytosolic protein. |
| F, G | AT1G78380 | GSTU19, glutathione | 2.583760756 | 0.002 | glutathione transferase that is a member of Tau GST |
| | | S-transferase TAU 19 | | | gene family. Expression is induced by drought stress, |
| | | | | | oxidative stress, and high doses of auxin and |
| | | | | | cytokinin. naming convention according to Wagner et |
| | | | | | al. (2002) The expression of this gene is upregulated |
| | | | | | by herbicide safeners such as benoxacor and |
| | | | | | fenclorim. |
| F, G | AT2G29720 | CTF2B, FAD/NAD(P)- | 3.145972448 | 0.026 | FAD/NAD(P)-binding oxidoreductase family protein |
| | | binding oxidoreductase | | | |
| | | family protein | | | |
| F, G | AT3G11340 | UDP- | 3.032097566 | 0.04 | glucosyltransferase that conjugates isoleucic acid and |
| | | Glycosyltransferase | | | modulates plant defense and senescence. |
| | | superfamily protein | | | |
| F, G | AT3G26830 | PAD3, Cytochrome | 14.61448668 | 0.00004092 | Mutations in pad3 are defective in biosynthesis of the |
| | | P450 superfamily | | | indole derived phytoalexin camalexin. cytochrome |
| | | protein | | | P450 enzyme that catalyzes the conversion of |
| | | | | | dihydrocamalexic acid to camalexin. The mRNA is |
| | | | | | cell-to-cell mobile. |
| F, G | AT4G31500 | ATR4, CYP83B1, | 2.418188216 | 0.046 | an oxime-metabolizing enzyme in the biosynthetic |
| | | RED1, RNT1, SUR2, | | | pathway of glucosinolates. Is required for |
| | | cytochrome P450, | | | phytochrome signal transduction in red light. Mutation |
| | | family 83, subfamily B, | | | confers auxin overproduction. |
| | | polypeptide 1 | | | |
| F, G | AT4G34135 | UGT73B2, UDP- | 8.082080074 | 0.003 | The At4g34135 gene flavonol 7-O-glucosyltransferase |
| | | glucosyltransferase | | | (EC 2.4.1.237) that glucosylates also with a 20 fold |
| | | 73B2 | | | lower activity flavonols (kaempferol and quercetin) at |
| | | | | | the 3-O-position. |
| F, G | AT4G37760 | SQE3, squalene | 50.11829138 | 0.014 | squalene epoxidase 3 |
| | | epoxidase 3 | | | |
| F, G | AT5G03630 | ATMDAR2, Pyridine | 2.748603928 | 0.012 | Pyridine nucleotide-disulfide oxidoreductase family |
| | | nucleotide-disulphide | | | protein |
| | | oxidoreductase family | | | |
| | | protein | | | |
| F, G | AT5G18170 | GDH1, glutamate | 86.38984437 | 0.016 | the 43 kDa alpha-subunit of the glutamate |
| | | dehydrogenase 1 | | | dehydrogenase with a putative mitochondrial transit |
| | | | | | polypeptide and NAD(H)− and alpha-ketoglutarate- |
| | | | | | binding domains. Mitochondrial localization |
| | | | | | confirmed by subcellular fractionation. Combines in |
| | | | | | several ratios with GDH2 protein (GDH-beta) to form |
| | | | | | seven isoenzymes. Catalyzes the cleavage of glycine |
| | | | | | residues. May be involved in ammonia assimilation |
| | | | | | under conditions of inorganic nitrogen excess. The |
| | | | | | enzyme is almost exclusively found in the |
| | | | | | mitochondria of stem and leaf companion cells. |
| F, G | AT5G24530 | DMR6, 2-oxoglutarate | 15.5761431 | 0.00002479 | putative 2OG-Fe(II) oxygenase that is defense- |
| | | (2OG) and Fe(II)- | | | associated but required for susceptibility to downy |
| | | dependent oxygenase | | | mildew. The mRNA is cell-to-cell mobile. |
| | | superfamily protein | | | |
| F, G | AT5G57800 | CER3, FLP1, WAX2, | 3321.575875 | 0.002 | transmembrane protein with similarity to the sterol |
| | | YRE, Fatty acid | | | desaturase family at the N-terminus and to the short- |
| | | hydroxylase | | | chain dehydrogenase/reductase family at the C- |
| | | superfamily | | | terminus. Mutant analyses indicate this protein is |
| | | | | | involved in cuticle membrane and wax biosynthesis. |
| | | | | | The mRNA is cell-to-cell mobile. |
| F, G | AT4G37990 | ELI3-2, elicitor- | 56.97769729 | 0.002 | an aromatic alcohol: NADP+ oxidoreductase whose |
| | | activated gene 3-2 | | | mRNA levels are increased in response to treatment |
| | | | | | with a variety of phytopathogenic bacteria. Though |
| | | | | | similar to mannitol dehydrogenases, this enzyme does |
| | | | | | not have mannitol dehydrogenase activity. |
| D, E, G | AT1G16890 | UBC36, ubiquitin- | 3.951137725 | 0.03 | UBC36/UBC13B protein that may play a role in DNA |
| | | conjugating enzyme 36 | | | damage responses and error-free post-replicative DNA |
| | | | | | repair. It can bind to the MMZ/UEV1 proteins in vitro. |
| D, E, G | AT2G30110 | UBA1, ubiquitin- | 3.321653124 | 0.003 | ubiquitin-activating enzyme (E1), involved in the first |
| | | activating enzyme 1 | | | step in conjugating multiple ubiquitins to proteins |
| | | | | | targeted for degradation. Gene is expressed in most |
| | | | | | tissues examined. Mutant is able to revert the |
| | | | | | constitutive defense responses phenotype of snc1, |
| | | | | | which indicates the gene is involved in defense |
| | | | | | response. It also indicates that ubiquitination plays a |
| | | | | | role in plant defense signalling. |
| D, E, G | AT4G21100 | DDB1B, damaged | 1615.021625 | 0.0003354 | One of two closely related genes similar to a damaged |
| | | DNA binding protein | | | DNA binding protein originally described in |
| | | 1B | | | mammals. May form a complex with DET1 to regulate |
| | | | | | photomorphogenesis. Loss of function mutations are |
| | | | | | lethal. The DDB lb protein binds with a number of |
| | | | | | DWD-containing proteins and may form part of a |
| | | | | | CUL4-based E3 ubiquitin ligase. |
| D, E, G | AT5G49570 | PNG1, peptide-N- | 3.133826988 | 0.032 | protein that has peptide: N-glycanase activity in |
| | | glycanase 1 | | | enzymatic assay in heterologous systems (although the |
| | | | | | activity was not detected in wild-type plants). |
| C, E, G | AT1G08420 | BSL2, BRI1 suppressor | 2.073465041 | 0.037 | BRI1 suppressor 1 (BSU1)-like 2 |
| | | 1 (BSU1)-like 2 | | | |
| E, G | AT1G21210 | WAK4, wall associated | 8.976150857 | 0.007 | cell wall-associated ser/thr kinase involved in cell |
| | | kinase 4 | | | elongation and lateral root development |
| E, G | AT1G48210 | Protein kinase | 6.14810165 | 0.003 | Protein kinase superfamily protein |
| | | superfamily protein | | | |
| E, G | AT1G51660 | ATMEK4, ATMKK4, | 2.617942408 | 0.003 | mitogen-activated map kinase kinase (there are nine in |
| | | MKK4, mitogen- | | | Arabidopsis) involved in innate immunity. This protein |
| | | activated protein kinase | | | activates MPK3/MPK6 and early-defense genes |
| | | kinase 4 | | | redundantly with MKK5. In plants with both MKK5 |
| | | | | | and MKK4 levels reduced by RNAi plants, floral |
| | | | | | organs do not abscise suggestion a role for both |
| | | | | | proteins in mediating floral organ abscission. The |
| | | | | | mRNA is cell-to-cell mobile. |
| E, G | AT2G02560 | CAND1, cullin- | 42.10100914 | 0.04 | Arabidopsis thaliana homolog of human CAND1 |
| | | associated and | | | (cullin-associated and neddylation-dissociated). |
| | | neddylation dissociated | | | Putative similarity to TBP-interacting protein TIP120. |
| | | | | | Ubiquitously expressed in plant tissues throughout |
| | | | | | development. T-DNA insertion mutant plants were |
| | | | | | completely sterile and resistant to sirtinol and auxin, |
| | | | | | but not to gibberellins or brassinolide. Displayed |
| | | | | | developmental phenotypes similar to those of axr1, |
| | | | | | namely, short petioles, downwardly curling leaves, |
| | | | | | shorter inflorescence. Required for SCF function and |
| | | | | | appears to modulate SCF complex cycling. Physically |
| | | | | | interacts with CUL1. The mRNA is cell-to-cell mobile. |
| E, G | AT2G16600 | ROC3, rotamase CYP | 5.751533228 | 0.028 | cytosolic cyclophilin ROC3. The mRNA is cell-to-cell |
| | | 3 | | | mobile. |
| E, G | AT2G37710 | RLK, receptor lectin | 4.873064424 | 0.016 | Induced in response to Salicylic acid. The mRNA is |
| | | kinase | | | cell-to-cell mobile. |
| E, G | AT3G09350 | Fes1A | 3.16462928 | 0.014 | one of the Arabidopsis orthologs of the human Hsp70- |
| | | | | | binding protein 1 (HspBP-1) and yeast Fes1p: Fes1A |
| | | | | | (AT3G09350), Fes1B (AT3G53800), Fes1C |
| | | | | | (AT5G02150). Fes1A is cytosolic and associates with |
| | | | | | cytosolic Hsp70. Mutants showed increased heat- |
| | | | | | sensitive phenotype suggestion the involvement of |
| | | | | | Fes1A in acquired thermotolerance. Does not have |
| | | | | | nucleotide exchange factor activity in vitro. |
| E, G | AT3G12580 | HSP70, heat shock | 4.719822467 | 0.006 | heat shock protein 70 |
| | | protein 70 | | | |
| E, G | AT3G14840 | Leucine-rich repeat | 2.097211832 | 0.014 | LRR-RLK protein that is localized to the plasma |
| | | transmembrane protein | | | membrane and is involved in regulation of plant innate |
| | | kinase | | | immunity to microbes. LIK1 is phosphorylated by |
| | | | | | CERK1, a kinase involved in chitin perception. The |
| | | | | | mRNA is cell-to-cell mobile. |
| E, G | AT3G19420 | PTEN 2 | 2.241160922 | 0.016 | phosphatase with low in vitro tyrosine phosphatase |
| | | | | | activity that is capable of dephosphorylating in vitro |
| | | | | | the 3′phosphate group of PI3P, PI(3,4)P2, and |
| | | | | | PI(3,5)P2 and may be an effector of lipid signaling. |
| | | | | | The mRNA is cell-to-cell mobile. |
| E, G | AT3G48990 | AMP-dependent | 2.006176223 | 0.017 | an oxalyl-CoA synthetase and is required for oxalate |
| | | synthetase and ligase | | | degradation, for normal seed development, and for |
| | | family protein | | | defense against an oxalate-producing fungal pathogen. |
| E, G | AT4G15802 | AtHSBP, HSBP, heat | 6.218241496 | 0.0001123 | protein with similarity to heat shock factor binding |
| | | shock factor binding | | | proteins. Involved in negative regulation of heat shock |
| | | protein | | | response. Becomes nuclear localized upon heat |
| | | | | | treatment. |
| E, G | AT4G26070 | MEK1 | 4.030833705 | 0.028 | Member of MAP Kinase Kinase. Likely functions in a |
| | | | | | stress-activated MAPK pathway. Can phosphorylate |
| | | | | | the MAPK AtMPK4, in response to stress. Gets |
| | | | | | phosphorylated by MEKK1 in response to wounding. |
| E, G | AT4G26120 | NPR2, NPR1-like | 16.69681087 | 0.003 | Ankyrin repeat family protein/BTB/POZ domain- |
| | | protein 2 | | | containing protein |
| E, G | AT4G39090 | RD19A, Papain family | 3.140839158 | 0.031 | Similar to cysteine proteinases, induced by desiccation |
| | | cysteine protease | | | but not abscisic acid. Required for RRS1-R mediated |
| | | | | | resistance against Ralstonia solanacearum. Interacts |
| | | | | | with the R. solanacearum type III effector PopP2. |
| | | | | | RD19 associates with PopP2 to form a nuclear |
| | | | | | complex that is required for activation of the RRS1- |
| | | | | | R?mediated resistance response. |
| E, G | AT5G02490 | Heat shock protein 70 | 2.202605625 | 0.0003584 | Heat shock protein 70 (Hsp 70) family protein |
| | | (Hsp 70) family protein | | | |
| E, G | AT5G14250 | FUS11, Proteasome | 2.75440381 | 0.004 | subunit 3 of the COP9 signalosome. |
| | | component (PCI) | | | |
| | | domain protein | | | |
| E, G | AT5G43060 | Granulin repeat | 4.976365059 | 0.01 | Peptidase, activity detected in extracts of root, leaf and |
| | | cysteine protease | | | cell culture. |
| | | family protein | | | |
| E, G | AT5G45110 | NPR3, NPR1-like | 9.396321195 | 0.0002424 | NPR3, a paralog of NPR1. Involved in negative |
| | | protein 3 | | | regulation of defense responses against bacterial and |
| | | | | | oomycete pathogens. npr3 mutants has elevated level |
| | | | | | of PR1 expression. Interacts with TGA2, TGA3, |
| | | | | | TGA5 and TGA6 in yeast two hybrid assays. NPR3 |
| | | | | | and NPR4 are receptors for the immune signal |
| | | | | | salicylic acid. The mRNA is cell-to-cell mobile. |
| B, D, G | AT5G35530 | Ribosomal protein S3 | 3.141460686 | 0.024 | Ribosomal protein S3 family protein |
| | | family protein | | | |
| D, G | AT2G29570 | PCNA2, proliferating | 2.831339329 | 0.046 | Functionally interacts with POLH to repair DNA |
| | | cell nuclear antigen 2 | | | damaged by UVB damage. May be sumoylated. |
| D, G | AT3G06010 | ATCHR12, Homeotic | 9.34235423 | 0.018 | AtCHR12, a SNF2/Brahma-type chromatin- |
| | | gene regulator | | | remodeling protein. AtCHR12 mediates temporary |
| | | | | | growth arrest in Arabidopsis upon perceiving |
| | | | | | environmental stress. |
| D, G | AT3G07930 | DNA glycosylase | 521.3600823 | 0.000147 | DNA glycosylase superfamily protein |
| | | superfamily protein | | | |
| D, G | AT3G14890 | Phosphoesterase | 3.845087005 | 0.043 | base excision repair protein that, together with APE2, |
| | | | | | it plays overlapping roles in the maintenance of |
| | | | | | epigenome and genome stability in plants. |
| C, G | AT4G13350 | NSP (nuclear shuttle | 4.269177143 | 0.009 | GTPase that interacts with nuclear shuttle proteins |
| | | protein)-interacting | | | (NSPs) from a number of different plant viruses. The |
| | | GTPase | | | gene is widely expressed and NIG transcript levels do |
| | | | | | not rise in response to viral infection. This cytoplasmic |
| | | | | | protein does not directly interact with a viral |
| | | | | | movement protein (MP), but, it does promote the |
| | | | | | movement of NSP from the nucleus to the cytoplasm. |
| | | | | | Overexpression of NIG in Arabidopsis plants renders |
| | | | | | them more sensitive to geminivirus infection. |
| C, G | AT1G01960 | EDA10 | 2.220715332 | 0.03 | one of the functionally redundant ARF guanine- |
| | | | | | nucleotide exchange factors (ARF-GEFs). Functions |
| | | | | | as regulators of post-Golgi trafficking. |
| C, G | AT4G02510 | TOC86, translocon at | 3.610006467 | 0.024 | An integral membrane GTPase that functions as a |
| | | the outer envelope | | | transit-sequence receptor required for the import of |
| | | membrane of | | | proteins necessary for chloroplast biogenesis. Located |
| | | chloroplasts 159 | | | in the outer chloroplast membrane. Phosphorylation of |
| | | | | | the G-domains regulate translocon assembly. The |
| | | | | | mRNA is cell-to-cell mobile. |
| B, G | AT2G34480 | Ribosomal protein | 3.349837664 | 0.006 | nuclear localized member of the ribosomal L18ae/LX |
| | | L18ae/LX family | | | protein family. Loss of function mutations show |
| | | protein | | | reduced transmission through the gametophytes and |
| | | | | | embryo lethality. |
| G | AT1G02450 | NIMIN1 | 17.71850982 | 0.0002129 | NIMIN1 modulates PR gene expression according the |
| | | | | | following model: NPR1 forms a ternary complex with |
| | | | | | NIMIN1 and TGA factors upon SAR induction that |
| | | | | | binds to a positive regulatory cis-element of the PR-1 |
| | | | | | promoter, termed LS7. This leads to PR-1 gene |
| | | | | | induction. NIMIN1 decreases transcriptional |
| | | | | | activation, possibly through its EAR motif, which |
| | | | | | results in fine-tuning of PR-1 gene expression. |
| G | AT1G55450 | S-adenosyl-L- | 2.240481943 | 0.016 | S-adenosyl-L-methionine-dependent |
| | | methionine-dependent | | | methyltransferases superfamily protein |
| | | methyltransferases | | | |
| | | superfamily protein | | | |
| G | AT1G60140 | TPS10, trehalose | 2102.433 | 0.001 | an enzyme putatively involved in trehalose |
| | | phosphate synthase | | | biosynthesis. The protein has a trehalose synthase |
| | | | | | (TPS)-like domain that may or may not be active as |
| | | | | | well as a trehalose phosphatase (TPP)-like domain. |
| G | AT1G66090 | Disease resistance | 6.25603175 | 0.00001173 | Disease resistance protein (TIR-NBS class) |
| | | protein (TIR-NBS | | | |
| | | class) | | | |
| G | AT1G72910 | Toll-Interleukin- | 2.606979946 | 0.00047 | Toll-Interleukin-Resistance (TIR) domain-containing |
| | | Resistance (TIR) | | | protein |
| | | domain-containing | | | |
| | | protein | | | |
| G | AT2G03440 | NRP1, nodulin-related | 14.30361122 | 0.039 | Induced at the transcriptional level by Pseudomonas |
| | | protein 1 | | | syringae pv. tomato infection. |
| G | AT2G14560 | LURP1, Protein of | 4.630490539 | 0.038 | LURP1, a member of the LURP cluster (late |
| | | unknown function | | | upregulated in response to Hyaloperonospora |
| | | (DUF567) | | | parasitica) which exhibits a pronounced upregulation |
| | | | | | after recognition of the pathogenic oomycte |
| | | | | | H. parasitica. LURP1 is required for full basal defense to |
| | | | | | H. parasitica and resistance to this pathogen mediated |
| | | | | | by the R-proteins RPP4 and RPP5. The mRNA is cell- |
| | | | | | to-cell mobile. |
| G | AT2G14610 | PR1, pathogenesis- | 3.64837311 | 0.0002666 | PR1 gene expression is induced in response to a |
| | | related gene 1 | | | variety of pathogens. It is a useful molecular marker |
| | | | | | for the SAR response. Though the Genbank record for |
| | | | | | the cDNA associated to this gene is called ‘PR-1-like’, |
| | | | | | the sequence actually corresponds to PR1. Expression |
| | | | | | of this gene is salicylic-acid responsive. |
| G | AT2G17265 | DMR1, HSK, | 2.162821903 | 0.0007399 | homoserine kinase (HSK) which produces O-phospho- |
| | | homoserine kinase | | | L-homoserine (HserP), a compound at the branching |
| | | | | | point of methionine and threonine biosynthesis. HSK |
| | | | | | is found in the stromal fraction of chloroplasts. |
| | | | | | Mutation of this gene results in higher level of the |
| | | | | | amino acid homoserine and resistance to downy |
| | | | | | mildew pathogen Hyaloperonospora arabidopsidis. |
| G | AT2G24850 | TAT3, tyrosine | 18.45478915 | 0.027 | tyrosine aminotransferase that is responsive to |
| | | aminotransferase 3 | | | treatment with jasmonic acid. |
| G | AT2G26560 | PLP2, phospholipase A | 3.961195476 | 0.043 | lipid acyl hydrolase with wide substrate specificity that |
| | | 2A | | | accumulates upon infection by fungal and bacterial |
| | | | | | pathogens. Protein is localized in the cytoplasm in |
| | | | | | healthy leaves, and in membranes in infected cells. |
| | | | | | Plays a role in cell death and differentially affects the |
| | | | | | accumulation of oxylipins. Contributes to resistance to |
| | | | | | virus. |
| G | AT2G37970 | SOUL-1, SOUL heme- | 2.926002428 | 0.031 | SOUL heme-binding family protein |
| | | binding family protein | | | |
| G | AT3G01290 | SPFH/Band 7/PHB | 6.530545545 | 0.002 | SPFH/Band 7/PHB domain-containing membrane- |
| | | domain-containing | | | associated protein family |
| | | membrane-associated | | | |
| | | protein family | | | |
| G | AT3G04210 | Disease resistance | 7.601551566 | 0.017 | TN13 is a TIR-NBS protein involved in immune |
| | | protein (TIR-NBS | | | response. It co localizes with the ER and perinuclear |
| | | class) | | | membranes and interacts with MOS6. |
| G | AT3G18520 | HDA15, histone | 2572.7625 | 0.002 | protein with similarity to histone deacetylases. Plants |
| | | deacetylase 15 | | | expressing RNAi directed against this gene show a |
| | | | | | moderate resistance to agrobacterium-mediated root |
| | | | | | transformation. |
| G | AT3G43810 | CAM7, calmodulin 7 | 3.958075882 | 0.002 | EF hand domain protein calmodulin. Can functionally |
| | | | | | complement a yeast CaM mutant. |
| G | AT3G44480 | RPP1, Disease | 13.72108266 | 0.025 | TIR-NB-LRR R-protein RPP1 that confers resistance |
| | | resistance protein | | | to Peronospora parasitica (downy mildew). |
| | | (TIR-NBS-LRR class) | | | |
| | | family | | | |
| G | AT3G48090 | EDS1, enhanced | 3.176849719 | 0.008 | Component of R gene-mediated disease resistance in |
| | | disease susceptability | | | Arabidopsis thaliana with homology to eukaryotic |
| | | | | | lipases. |
| G | AT3G50930 | BCS1, cytochrome | 15.97249988 | 0.006 | protein that is present in a homo-multimeric protein |
| | | BC1 synthesis | | | complex on the outer mitochondrial membrane and |
| | | | | | plays a role in cell death and amplifying salicylic acid |
| | | | | | signalling. The mRNA is cell-to-cell mobile. |
| G | AT3G51250 | Senescence/dehydration- | 26.13060967 | 0.034 | Senescence/dehydration-associated protein-like protein |
| | | associated protein- | | | |
| | | related | | | |
| G | AT3G52430 | ATPAD4, PAD4, | 3.227623612 | 0.022 | lipase-like gene that is important for salicylic acid |
| | | alpha/beta-Hydrolases | | | signaling and function in resistance (R) gene-mediated |
| | | superfamily protein | | | and basal plant disease resistance. PAD4 can interact |
| | | | | | directly with EDS1, another disease resistance |
| | | | | | signaling protein. Expressed at elevated level in |
| | | | | | response to green peach aphid (GPA) feeding, and |
| | | | | | modulates the GPA feeding-induced leaf senescence |
| | | | | | through a mechanism that doesn't require camalexin |
| | | | | | synthesis and salicylic acid (SA) signaling. Required |
| | | | | | for the ssi2-dependent heightened resistance to GPA. |
| | | | | | The mRNA is cell-to-cell mobile. |
| G | AT4G16950 | RPP5, Disease | 3.79227619 | 0.044 | Contains a putative nucleotide binding site and |
| | | resistance protein | | | leucine-rich repeats. Similar to the plant resistance |
| | | (TIR-NBS-LRR class) | | | genes N and L6, and to the toll and interleukin-1 |
| | | | | | receptors. Confers resistance to Peronospora |
| | | | | | parasitica. Redundant function together with SIKIC1 |
| | | | | | and 3 in SNC1-mediated autoimmunity. Protein levels |
| | | | | | controlled by MUSE1 and MUSE2. |
| G | AT4G19510 | Disease resistance | 5.174685292 | 0.003 | Disease resistance protein (TIR-NBS-LRR class) |
| | | protein (TIR-NBS- | | | |
| | | LRR class) | | | |
| G | AT4G33050 | EDA39, calmodulin- | 3.102716934 | 0.033 | calmodulin-binding protein involved in stomatai |
| | | binding family protein | | | movement |
| G | AT5G41750 | Disease resistance | 10.2128062 | 0.0008359 | Disease resistance protein (TIR-NBS-LRR class) |
| | | protein (TIR-NBS- | | | family |
| | | LRR class) | | | |
| G | AT5G45510 | Leucine-rich repeat | 5.051626678 | 0.0001342 | Leucine-rich repeat (LRR) family protein |
| | | (LRR) family protein | | | |
| G | AT5G45520 | Leucine-rich repeat | 2274.360875 | 0.006 | Leucine-rich repeat (LRR) family protein |
| | | (LRR) family protein | | | |
| G | AT5G48620 | Disease resistance | 2.826581182 | 0.016 | Disease resistance protein (CC-NBS-LRR class) |
| | | protein (CC-NBS-LRR | | | family |
| | | class) | | | |
| G | AT5G54310 | AGD5, NEV, ARF- | 8.653446694 | 0.019 | A member of ARF GAP domain (AGD), A thaliana |
| | | GAP domain 5 | | | has 15 members, grouped into four classes. Regulates |
| | | | | | membrane trafficking and organ separation. |
| G | AT5G57560 | TCH4, XTH22, | 102.3521183 | 0.033 | cell wall-modifying enzyme, rapidly upregulated in |
| | | Xyloglucan | | | response to environmental stimuli. |
| | | endotransglucosylase/ | | | |
| | | hydrolase family protein | | | |
| G | AT5G45500 | RNI-like superfamily | 2.930585074 | 0.002 | RNI-like superfamily protein. Leucine-Rich repeat |
| | | protein | | | (LRR) family protein |
| C, E, F | AT1G68730 | Zim17-type zinc finger | 11.82585661 | 0.04 | Zim17-type zinc finger protein |
| | | protein | | | |
| B, E, F | AT1G68200 | Zinc finger C-x8-C-x5- | 3.854512325 | 0.006 | Zinc finger C-x8-C-x5-C-x3-H type family protein |
| | | C-x3-H type family | | | |
| | | protein | | | |
| D, F | AT2G21790 | RNR1, ribonucleotide | 2182.409958 | 0.0007951 | large subunit of ribonucleotide reductase involved in |
| | | reductase 1 | | | the production of deoxyribonucleoside triphosphates |
| | | | | | (dNTPs) for DNA replication and repair |
| F | AT1G05720 | Selenoprotein family | 2.426087484 | 0.04 | selenoprotein family protein |
| | | protein | | | |
| F | AT1G11680 | CYP51, | 2.795984794 | 0.006 | putative obtusifoliol 14-alpha demethylase involved in |
| | | CYTOCHROME P450 | | | sterol biosynthesis. The mRNA is cell-to-cell mobile. |
| | | 51G1 | | | |
| F | AT1G16350 | Aldolase-type TIM | 65.91134451 | 0.042 | Aldolase-type TIM barrel family protein |
| | | barrel family protein | | | |
| F | AT1G22400 | UGT85A1, UDP- | 4.863075405 | 0.03 | UDP-Glycosyltransferase superfamily protein |
| | | Glycosyltransferase | | | |
| | | superfamily protein | | | |
| F | AT1G48320 | Thioesterase | 6.589263193 | 0.031 | one of the two functional DHNA-CoA (1,4-dihydroxy- |
| | | superfamily protein | | | 2-naphthoyl-CoA) thioesterases found in Arabidopsis. |
| F | AT1G70580 | AOAT2, GGT2, | 10.47365578 | 0.03 | protein with glyoxylate aminotransferase activity. It |
| | | alanine-2-oxoglutarate | | | can act on a number of different small substrates and |
| | | aminotransferase 2 | | | amino acids in vitro. |
| F | AT2G01490 | Phytanoyl-CoA | 2.659254608 | 0.032 | phytanoyl-CoA 2-hydroxylase (PAHX). The mRNA is |
| | | dioxygenase (PhyH) | | | cell-to-cell mobile. |
| | | family protein | | | |
| F | AT2G26400 | ARD3, acireductone | 8.569790647 | 0.005 | protein predicted to belong to the acireductone |
| | | dioxygenase 3 | | | dioxygenase (ARD/ARD?)family. |
| F | AT2G29320 | NAD(P)-binding | 40.03724019 | 0.045 | NAD(P)-binding Rossmann-fold superfamily protein |
| | | Rossmann-fold | | | |
| | | superfamily protein | | | |
| F | AT2G43820 | UGT74F2, UDP- | 8.985932916 | 0.011 | nicotinate-O-glycosyltransferase. Induced by Salicylic |
| | | glucosyltransferase | | | acid, virus, fungus and bacteria. Also involved in the |
| | | 74F2 | | | tryptophan synthesis pathway. Independent of NPR1 |
| | | | | | for their induction by salicylic acid. UGT74F1 |
| | | | | | transfers UDP: glucose to salicylic acid (forming a |
| | | | | | glucoside (SAG) and a glucose ester (SGE)), benzoic |
| | | | | | acid, and anthranilate in vitro. UGT74F2 shows a |
| | | | | | weak ability to catalyze the formation of the p- |
| | | | | | aminobenzoate-glucose ester in vitro. But, UGT75B1 |
| | | | | | appears to be the dominant pABA |
| | | | | | acylglucosyltransferase in vivo based on assays in |
| | | | | | leaves, flowers, and siliques. |
| F | AT4G13180 | NAD(P)-binding | 5.4597349 | 0.005 | NAD(P)-binding Rossmann-fold superfamily protein |
| | | Rossmann-fold | | | |
| | | superfamily protein | | | |
| F | AT4G22220 | ISU1, SufE/NifU | 3.436677001 | 0.042 | mitochondrial protein similar to E. coli IscU. In |
| | | family protein | | | bacteria, IscU is a scaffold protein accepting sulfur and |
| | | | | | iron to build a transient Fe—S cluster, which is |
| | | | | | subsequently transferred to a target apoprotein. |
| F | AT5G14240 | Thioredoxin | 32.0699871 | 0.044 | Thioredoxin superfamily protein |
| | | superfamily protein | | | |
| F | AT5G22140 | FAD/NAD(P)-binding | 9.815053313 | 0.0002677 | FAD/NAD(P)-binding oxidoreductase family protein |
| | | oxidoreductase family | | | |
| | | protein | | | |
| F | AT5G40760 | G6PD6, glucose-6- | 4.098820827 | 0.035 | cytosolic glucose-6-phosphate dehydrogenase that is |
| | | phosphate | | | insensitive to reduction by DTT and whose mRNA is |
| | | dehydrogenase 6 | | | expressed ubiquitously. The mRNA is cell-to-cell |
| | | | | | mobile. |
| F | AT5G48180 | NSP5, nitrile specifier | 5235.696 | 0.0009395 | nitrile-specifier protein NSP5. NSP5 is one out of five |
| | | protein 5 | | | (At3g16400/NSP1, At2g33070/NSP2, |
| | | | | | At3g16390/NSP3, At3g16410/NSP4 and |
| | | | | | At5g48180/NSP5) A. thaliana epithiospecifier protein |
| | | | | | (ESP) homologues that promote simple nitrile, but not |
| | | | | | epithionitrile or thiocyanate formation. |
| C, E | AT5G47040 | LON2, lon protease 2 | 2.472454332 | 0.002 | member of the Lon protease-like proteins |
| | | | | | (Lon1/At5g26860, Lon2/At5g47040, |
| | | | | | Lon3/At3g05780, Lon4/At3g05790). Lon is a |
| | | | | | multifunctional ATP-dependent protease which exists |
| | | | | | in bacteria, archaea and within organelles in eukaryotic |
| | | | | | cells. Lon proteases are responsible for the degradation |
| | | | | | of abnormal, damaged and unstable proteins. |
| C, E | AT3G18060 | Transducin family | 7.1893436 | 0.017 | transducin family protein/WD-40 repeat family |
| | | protein/WD-40 repeat | | | protein. Cul4-RING E3 ubiquitin ligase complex |
| | | family protein | | | |
| C, E | AT2G31200 | ADF6 | 2.256109133 | 0.011 | actin depolymerizing factor 6 (ADF6). The mRNA is |
| | | | | | cell-to-cell mobile. |
| E | AT1G04860 | UBP2, ubiquitin- | 2.306075545 | 0.0007404 | ubiquitin-specific protease. |
| | | specific protease 2 | | | |
| E | AT1G08050 | Zinc finger (C3HC4- | 7.543438856 | 0.004 | Zinc finger (C3HC4-type RING finger) family protein |
| | | type RING finger) | | | |
| | | family protein | | | |
| E | AT2G04430 | atnudt5, NUDT5, | 44.14867451 | 0.029 | nudix hydrolase homolog 5 |
| | | nudix hydrolase | | | |
| | | homolog 5 | | | |
| E | AT2G20190 | CLIP-associated | 2554.278083 | 0.00008309 | microtubule-associated protein that is involved in both |
| | | protein | | | cell division and cell expansion. It likely promotes |
| | | | | | microtubule stability. |
| E | AT2G21470 | SAE2, SUMO- | 5.816764678 | 0.041 | one of the two subunits of the SUMO activation |
| | | activating enzyme 2 | | | enzyme required during sumolation. Sumolation is a |
| | | | | | post-translational protein modification process similar |
| | | | | | to ubiquitination during which a polypeptide (SUMO) |
| | | | | | is covalently attached to a target protein. |
| E | AT2G28450 | zinc finger (CCCH- | 2.94745402 | 0.012 | zinc finger (CCCH-type) family protein |
| | | type) family protein | | | |
| E | AT2G32160 | S-adenosyl-L- | 3.438645919 | 0.012 | S-adenosyl-L-methionine-dependent |
| | | methionine-dependent | | | methyltransferases superfamily protein |
| | | methyltransferases | | | |
| | | superfamily protein | | | |
| E | AT2G47110 | UBQ6, ubiquitin 6 | 2.043646216 | 0.006 | polyubiquitin gene The mRNA is cell-to-cell mobile. |
| E | AT3G07990 | SCPL27, serine | 5807.184 | 0.007 | serine carboxypeptidase-like 27 |
| | | carboxypeptidase-like | | | |
| | | 27 | | | |
| E | AT3G20630 | UBP14, ubiquitin- | 5.017922504 | 0.003 | ubiquitin-specific protease. Identical to TTN6. Loss of |
| | | specific protease 14 | | | function mutations are embryo lethals, having |
| | | | | | development arrested at the preglobular/globular stage. |
| | | | | | Also involved in root responses to phosphate |
| | | | | | deficiency. |
| E | AT3G23570 | alpha/beta-Hydrolases | 2.728988268 | 0.002 | alpha/beta-Hydrolases superfamily protein |
| | | superfamily protein | | | |
| E | AT3G50590 | Transducin/WD40 | 3404.154333 | 0.001 | WD40/YVTN repeat protein. Autophagy pathway |
| | | repeat-like superfamily | | | regulation |
| | | protein | | | |
| E | AT3G58040 | SINAT2, seven in | 2.989448967 | 0.04 | RING finger domain containing protein that interacts |
| | | absentia of Arabidopsis | | | with AtRAP2.2. The mRNA is cell-to-cell mobile. |
| | | 2 | | | |
| E | AT4G01870 | tolB protein-related | 6.111279906 | 0.032 | tolB protein-like protein |
| E | AT4G17830 | Peptidase | 3.667358167 | 0.031 | NAOD functional acetylornithine deacetylase. |
| | | M20/M25/M40 family | | | Silenced lines plants flower early but have reduced |
| | | protein | | | fertility (siliques do not develop) as well as reduced |
| | | | | | ornithine levels. NAOD mediates a linear pathway for |
| | | | | | ornithine biosynthesis. |
| E | AT4G30890 | UBP24, ubiquitin- | 2578.715125 | 0.003 | ubiquitin-specific protease. |
| | | specific protease 24 | | | |
| E | AT5G60360 | SAG2, aleurain-like | 4.083440208 | 0.004 | senescence-associated thiol protease. The mRNA is |
| | | protease | | | cell-to-cell mobile. |
| E | AT5G60510 | Undecaprenyl | 7.40731429 | 0.006 | Undecaprenyl pyrophosphate synthetase family protein |
| | | pyrophosphate | | | |
| | | synthetase family | | | |
| | | protein | | | |
| E | AT5G61790 | ATCNX1, CNX1, | 5.259807906 | 0.008 | calnexin 1 |
| | | calnexin 1 | | | |
| E | AT5G67340 | ARM repeat | 203.1453606 | 0.014 | ARM repeat superfamily protein |
| | | superfamily protein | | | |
| E | AT3G15610 | Transducin/WD40 | 2.263771457 | 0.008 | Transducin/WD40 repeat-like superfamily protein |
| | | repeat-like superfamily | | | |
| | | protein | | | |
| E | AT3G45620 | Transducin/WD40 | 3.673690257 | 0.009 | This gene is predicted to encode a protein with a DWD |
| | | repeat-like superfamily | | | motif. It can bind to DDB la in Y2H assays, and |
| | | protein | | | DDB lb in co-IP assays, and may be involved in the |
| | | | | | formation of a CUL4-based E3 ubiquitin ligase |
| E | AT5G24710 | Transducin/WD40 | 2.012342931 | 0.036 | WD40/YVTN repeat protein. |
| | | repeat-like superfamily | | | |
| | | protein. Peptidase A1 | | | |
| | | domain-containing | | | |
| | | protein | | | |
| D | AT3G18580 | Nucleic acid-binding, | 6.349991253 | 0.01 | Member of the family of canonical mitochondrial |
| | | OB-fold-like protein | | | DNA binding proteins. Single-stranded binding protein |
| | | | | | which does not interfere with MMEJ. |
| D | AT4G31210 | DNA topoisomerase, | 2.583542458 | 0.041 | DNA topoisomerase, type IA, core |
| | | type IA, core | | | |
| C | AT2G41740 | VLN2, villin 2 | 3.542665946 | 0.041 | protein with high homology to animal villin. actin |
| | | | | | filament bundle assembly, actin filament capping |
| C | AT3G19960 | ATM1, myosin 1 | 2.331183156 | 0.023 | member of Myosin-like proteins |
| C | AT1G27970 | NTF2B, nuclear | 11.1592163 | 0.042 | an ortholog of yeast NTF2, a nuclear envelop transport |
| | | transport factor 2B | | | protein that functions as the nuclear import receptor |
| | | | | | for RanGDP, an essential player in nucleocytoplasmic |
| | | | | | transport. The mRNA is cell-to-cell mobile. |
| C | AT3G06720 | IMPA1, importin alpha | 2.092256022 | 0.018 | importin alpha involved in nuclear import. Protein |
| | | isoform 1 | | | interacts with Agrobacterium proteins VirD2 and |
| | | | | | VirE2. Is not individually essential for Agrobacterium- |
| | | | | | mediated root transformation, but when overexpressed |
| | | | | | can rescue the impa-4 decreased transformation |
| | | | | | susceptibility phenotype. |
| C | AT1G59610 | ADL3, CF1, DL3, | 2.502994433 | 0.003 | A high molecular weight GTPase whose GTP-binding |
| | | DRP2B, dynamin-like | | | domain shows a low homology to those of other plant |
| | | 3 | | | dynamin-like proteins. Contains a pleckstrin |
| | | | | | homologous domain. DRP2B and DRP1A participate |
| | | | | | together in clathrin-coated vesicle formation during |
| | | | | | endocytosis. The mRNA is cell-to-cell mobile. |
| C | AT3G13870 | RHD3, Root hair | 2.858612305 | 0.036 | required for regulated cell expansion and normal root |
| | | defective 3 GTP- | | | hair development, an evolutionarily conserved protein |
| | | binding protein | | | with putative GTP-binding motifs that is implicated in |
| | | (RHD3) | | | the control of vesicle trafficking between the |
| | | | | | endoplasmic reticulum and the Golgi compartments. |
| C | AT5G24520 | TTG1, | 3.341780748 | 0.017 | Required for the accumulation of purple anthocyanins |
| | | Transducin/WD40 | | | in leaves and stems. Involved in trichome and root hair |
| | | repeat-like superfamily | | | development. Controls epidermal cell fate |
| | | protein | | | specification Affects dihydroflavonol 4-reductase |
| | | | | | gene expression It is thought that a ternary complex |
| | | | | | composed of TT2, TT8 and TTG1 is necessary for |
| | | | | | correct expression of BAN in seed endothelium. Based |
| | | | | | on clonal analysis and other methonds TTG1 has been |
| | | | | | shown to act non-cell autonomously and to move via |
| | | | | | plasmodesmata between cells. Localization and levels |
| | | | | | of TTG1 affect patterning of leaf trichomes. Auxin and |
| | | | | | ethylene responsiveness of TTG1 transcription is lost |
| | | | | | in myb12 mutants. |
| C | AT1G07140 | SIRANBP, Pleckstrin | 709.313854 | 0.006 | putative Ran-binding protein (siRanBP). INVOLVED |
| | | homology (PH) domain | | | IN: intracellular transport, protein import into nucleus, |
| | | superfamily protein | | | translocation |
| C | AT3G19870 | AP-5 complex subunit | 4.590769246 | 0.016 | AP-5 complex subunit beta-like protein |
| | | beta-like protein | | | |
| C | AT3G28710 | ATPase, V0/A0 | 2.237991657 | 0.00007561 | ATPase, V0/A0 complex, subunit C/D |
| | | complex, subunit C/D | | | |
| C | AT5G05000 | TOC34, translocon at | 9.76131971 | 0.023 | Outer membrane GTPase protein that may function in |
| | | the outer envelope | | | import of nuclear encoded proteins into the |
| | | membrane of | | | chloroplast. Phosphorylation of the G-domains |
| | | chloroplasts 34 | | | regulate translocon assembly. |
| C | AT5G54750 | Transport protein | 2.898695804 | 0.0008123 | Part of multi-protein complex, acting as guanine |
| | | particle (TRAPP) | | | nucleotide exchange factors (GEFs) and possibly as |
| | | component | | | tethers, regulating intracellular trafficking. |
| B | AT1G01100 | 60S acidic ribosomal | 4.679633439 | 0.03 | Co-orthologous gene of large ribosomal subunit |
| | | protein family | | | protein RPP1. |
| B | AT3G61240 | DEA(D/H)-box RNA | 2.53199271 | 0.028 | DEA(D/H)-box RNA helicase family protein |
| | | helicase family protein | | | |
| B | AT5G64150 | RNA methyltransferase | 2.147414015 | 0.034 | RNA methyltransferase family protein |
| | | family protein | | | |
| B | AT1G12920 | ERF1-2, eukaryotic | 2.49554072 | 0.026 | eukaryotic release factor one homolog. |
| | | release factor 1-2 | | | |
| B | AT3G62870 | Ribosomal protein | 19.7434018 | 0.000333 | Ribosomal protein L7Ae/L30e/S12e/Gadd45 family |
| | | L7Ae/L30e/S12e/Gadd | | | protein |
| | | 45 family protein | | | |
| B | AT2G16360 | Ribosomal protein S25 | 2.055365138 | 0.003 | 40S ribosomal protein S25 |
| | | family protein | | | |
| B | AT2G31060 | Elongation factor | 3.320469781 | 0.001 | elongation factor family protein |
| | | family protein | | | |
| B | AT1G27900 | RNA helicase family | 1962.136292 | 0.002 | RNA helicase family protein |
| | | protein | | | |
| B | AT2G43410 | FPA, RNA binding | 4.866064994 | 0.018 | FPA is a gene that regulates flowering time in |
| | | | | | Arabidopsis via a pathway that is independent of |
| | | | | | daylength (the autonomous pathway). Mutations in |
| | | | | | FPA result in extremely delayed flowering. Double |
| | | | | | mutants with FCA have reduced fertility and |
| | | | | | single/double mutants have defects in siRNA mediated |
| | | | | | chromatin silencing. |
| B | AT3G62310 | RNA helicase family | 7.184690142 | 0.035 | RNA helicase family protein |
| | | protein | | | |
| B | AT4G09730 | RH39 | 46.88487933 | 0.029 | RH39, a DEAD-box protein involved in the |
| | | | | | introduction of the hidden break into the 23S rRNA in |
| | | | | | the chloroplasts. Recombinant RH39 binds to the 23S |
| | | | | | rRNA in a segment adjacent to the stem-loop creating |
| | | | | | the hidden break target loop in a sequence-dependent |
| | | | | | manner. Has ATP-hydrolyzing activity at a Kcat of 5.3/ |
| | | | | | min in the presence of rRNA sequence. Mutants have |
| | | | | | drastically reduced level of level of ribulose 1,5- |
| | | | | | bisphosphate carboxylase/oxygenase. The mRNA is |
| | | | | | cell-to-cell mobile. |
| B | AT5G62190 | PRH75, DEAD box | 5.900567132 | 0.027 | DEAD/DEAH box RNA helicase PRH75 |
| | | RNA helicase (PRH75) | | | |
| A | AT1G60095 | Mannose-binding lectin | 2.220346084 | 0.005 | Mannose-binding lectin superfamily protein |
| | | superfamily protein | | | |
| A | AT1G74940 | Protein of unknown | 1001.701844 | 0.019 | cyclin-dependent kinase, putative (DUF581) |
| | | function (DUF581) | | | |
| A | AT2G20010 | Protein of unknown | 2.965429985 | 0.028 | Gls protein (DUF810) |
| | | function (DUF810) | | | |
| A | AT2G28620 | P-loop containing | 7.174619085 | 0.0004092 | Mutants have radially swollen roots but do not exhibit |
| | | nucleoside triphosphate | | | defects in abundance or orientation of cortical |
| | | hydrolases superfamily | | | microtubules, nor are microfibrils reduced. Cellulose |
| | | protein | | | synthesis is also unchanged with respect to wild type. |
| | | | | | There is a disruption in the normal pattern of cell wall |
| | | | | | placement. |
| A | AT2G30500 | Kinase interacting | 66.93293597 | 0.027 | Kinase interacting (KIP1-like) family protein |
| | | (KIP1-like) family | | | |
| | | protein | | | |
| A | AT3G14075 | Mono-/di-acylglycerol | 2.342061405 | 0.027 | Mono-/di-acylglycerol lipase, N-terminal |
| | | lipase, N- | | | |
| | | terminal; Lipase, class 3 | | | |
| A | AT3G28510 | P-loop containing | 6.345095983 | 0.002 | P-loop containing nucleoside triphosphate hydrolases |
| | | nucleoside triphosphate | | | superfamily protein |
| | | hydrolases superfamily | | | |
| | | protein | | | |
| A | AT3G28540 | P-loop containing | 9.619894493 | 0.043 | P-loop containing nucleoside triphosphate hydrolases |
| | | nucleoside triphosphate | | | superfamily protein |
| | | hydrolases superfamily | | | |
| | | protein | | | |
| A | AT3G47630 | AT3G47630 | 703.3438853 | 0.002 | translocator assembly/maintenance protein |
| A | AT3G53470 | AT3G53470 | 4.352338413 | 0.034 | 2,3-bisphosphoglycerate-independent |
| | | | | | phosphoglycerate mutase |
| A | AT4G10120 | ATSPS4F, Sucrose- | 2.198730157 | 0.033 | protein with putative sucrose-phosphate synthase |
| | | phosphate synthase | | | activity. |
| | | family protein | | | |
| A | AT5G17760 | P-loop containing | 4791.223417 | 0.0009688 | P-loop containing nucleoside triphosphate hydrolases |
| | | nucleoside triphosphate | | | superfamily protein |
| | | hydrolases superfamily | | | |
| | | protein | | | |
| A | AT5G36225 | Zinc knuckle protein | 4753.56675 | 0.0005696 | zinc knuckle protein |
| A | AT5G51830 | pfkB-like carbohydrate | 4.220251395 | 0.018 | one of the several Arabidopsis fructokinases. |
| | | kinase family protein | | | Nomenclature according to Riggs 2017 has been |
| | | | | | adopted for the family by the community (personal |
| | | | | | communication, Boernke, Callis, Granot, Boernke, and |
| | | | | | Smeekens). Important for seed oil accumulation and |
| | | | | | vascular development. |

Functional Group Key
H = Defense Response/SA signaling. Cell Death
G = Redox metabolism
E = Protein modification and catabolism
D = DNA Damage Response
C = Protein transpoprt
B = RNA binding/translation
A = Unclassified (*)

SN Components - Part 2

| | | | | | Cellular |
|---|---|---|---|---|---|
| Gene ID | Protein Class | Protein Family | Molecular Function | Biological Process | Component |
| AT4G05420 | DNA DAMAGE-BINDING | damaged DNA- | damaged DNA binding(GO: 0003684) | proteasome-mediated | nucleus |
| | PROTEIN 1 | binding protein | | ubiquitin-dependent protein | (GO: 0005634) |
| | (PTHR10644: SF3) | (PC00086); | | catabolic process | |
| | | mRNA | | (GO: 0043161); protein | |
| | | polyadenylation | | ubiquitination (GO: 0016567) | |
| | | factor (PC00146) | | | |
| AT5G50340 | DNA REPAIR | serine protease | | recombinational repair | |
| | PROTEIN | (PC00203) | | (GO: 0000725) | |
| | RADA-LIKE | | | | |
| | PROTEIN | | | | |
| | (PTHR32472: SF10) | | | | |
| AT5G10450 | 14-3-3-LIKE | | | signal transduction | |
| | PROTEIN GF14 | | | (GO: 0007165) | |
| | LAMBDA | | | | |
| | (PTHR18860: SF98) | | | | |
| AT5G44070 | PHYTOCHELATIN | cysteine protease | catalytic activity, acting on a protein | cellular response to chemical | |
| | SYNTHASE | (PC00081) | (GO: 0140096); transferase activity, | stimulus (GO: 0070887); | |
| | (PTHR33447: SF2) | | transferring acyl groups (GO: 0016746) | peptide biosynthetic process | |
| | | | | (GO: 0043043); response to | |
| | | | | cadmium ion (GO: 0046686); | |
| | | | | response to copper ion | |
| | | | | (GO: 0046688); response to | |
| | | | | stress (GO: 0006950); | |
| | | | | response to toxic substance | |
| | | | | (GO: 0009636); secondary | |
| | | | | metabolite biosynthetic | |
| | | | | process (GO: 0044550) | |
| AT1G05010 | 1-AMINOCYCLOPROPANE-1- | | dioxygenase activity (GO: 0051213) | | |
| | CARBOXYLATE | | | | |
| | OXIDASE 4 | | | | |
| | (PTHR10209: SF383) | | | | |
| AT1G02930 | GLUTATHIONE | | anion binding(GO: 0043168); cofactor | glutathione metabolic process | cytoplasm |
| | S-TRANSFERASE | | binding(GO: 0048037); glutathione | (GO: 0006749) | (GO: 0005737) |
| | F6-RELATED | | transferase activity (GO: 0004364); | | |
| | (PTHR43900: SF47) | | peptide binding(GO: 0042277) | | |
| AT1G04980 | PROTEIN | | | | |
| | DISULFIDE- | | | | |
| | ISOMERASE A6 | | | | |
| | (PTHR45815: SF3) | | | | |
| AT1G54100 | ALPHA-AMINOADIPIC | dehydrogenase | oxidoreductase activity (GO: 0016491) | | |
| | SEMIALDEHYDE | (PC00092) | | | |
| | DEHYDROGENASE | | | | |
| | (PTHR43521: SF1) | | | | |
| AT1G63460 | GLUTATHIONE | peroxidase | oxidoreductase activity (GO: 0016491) | cellular response to chemical | cytosol |
| | PEROXIDASE 8- | (PC00180) | | stimulus (GO: 0070887); | (GO: 0005829) |
| | RELATED | | | response to toxic substance | |
| | (PTHR11592: SF27) | | | (GO: 0009636) | |
| AT1G76680 | NADPH | oxidoreductase | oxidoreductase activity (GO: 0016491) | | |
| | DEHYDROGENASE 2- | (PC00176) | | | |
| | RELATED | | | | |
| | (PTHR22893: SF110) | | | | |
| AT1G78380 | GLUTATHIONE | | glutathione transferase activity | glutathione metabolic process | cytoplasm |
| | S-TRANSFERASE | | (GO: 0004364) | (GO: 0006749) | (GO: 0005737) |
| | U19 | | | | |
| | (PTHR11260: SF585) | | | | |
| AT2G29720 | FAD/NAD(P)-BINDING | oxygenase | | | |
| | OXIDOREDUCTASE | (PC00177) | | | |
| | FAMILY | | | | |
| | PROTEIN | | | | |
| | (PTHR45934: SF9) | | | | |
| AT3G11340 | GLUCOSYLTRANSFERASE- | | UDP-glucosyltransferase activity | | intracellular membrane- |
| | LIKE PROTEIN- | | (GO: 0035251) | | bounded organelle |
| | RELATED | | | | (GO: 0043231) |
| | (PTHR11926: SF1220) | | | | |
| AT3G26830 | BIFUNCTIONAL | oxygenase | oxidoreductase activity, acting on paired | | |
| | DIHYDROCAMALEXATE | (PC00177) | donors, with incorporation or reduction of | | |
| | SYNTHASE/ | | molecular oxygen, NAD(P)H as one | | |
| | CAMALEXIN | | donor, and incorporation of one atom of | | |
| | SYNTHASE- | | oxygen (GO: 0016709) | | |
| | RELATED | | | | |
| | (PTHR24298: SF566) | | | | |
| AT4G31500 | CYTOCHROME | oxygenase | oxidoreductase activity, acting on paired | | |
| | P450 83B1 | (PC00177) | donors, with incorporation or reduction of | | |
| | (PTHR24298: SF400) | | molecular oxygen, NAD(P)H as one | | |
| | | | donor, and incorporation of one atom of | | |
| | | | oxygen(GO: 0016709) | | |
| AT4G34135 | UDP- | | UDP-glycosyltransferase activity | | intracellular membrane- |
| | GLUCOSYL | | (GO: 0008194) | | bounded organelle |
| | TRANSFERASE | | | | (GO: 0043231) |
| | 73B2-RELATED | | | | |
| | (PTHR11926: SF903) | | | | |
| AT4G37760 | SQUALENE | oxygenase | oxidoreductase activity, acting on paired | sterol biosynthetic process | endoplasmic |
| | EPOXIDASE 3 | (PC00177) | donors, with incorporation or reduction of | (GO: 0016126) | reticulum |
| | (PTHR10835: SF9) | | molecular oxygen, NAD(P)H as one | | (GO: 0005783); |
| | | | donor, and incorporation of one atom of | | plasma membrane |
| | | | oxygen (GO: 0016709) | | (GO: 0005886); |
| | | | | | vacuole |
| | | | | | (GO: 0005773) |
| AT5G03630 | MONODEHYDROASCORBATE | dehydrogenase | oxidoreductase activity (GO: 0016491) | nitrogen compound metabolic | |
| | REDUCTASE 2- | (PC00092); | | process (GO: 0006807); | |
| | RELATED | oxidase(PC00175); | | respiratory electron transport | |
| | (PTHR43557: SF13) | reductase | | chain (GO: 0022904) | |
| | | (PC00198) | | | |
| AT5G18170 | GLUTAMATE | dehydrogenase | oxidoreductase activity (GO: 0016491) | cellular amino acid catabolic | |
| | DEHYDROGENASE 1 | (PC00092) | | process (GO: 0009063) | |
| | (PTHR11606: SF30) | | | | |
| AT5G24530 | PROTEIN | | dioxygenase activity (GO: 0051213) | | |
| | DOWNY | | | | |
| | MILDEW | | | | |
| | RESISTANCE 6 | | | | |
| | (PTHR10209: SF177) | | | | |
| AT5G57800 | PROTEIN | oxidase | oxidoreductase activity (GO: 0016491) | | |
| | ECERIFERUM 3 | (PC00175) | | | |
| | (PTHR11863: SF66) | | | | |
| AT4G37990 | CINNAMYL | dehydrogenase | oxidoreductase activity, acting on the | aromatic compound | |
| | ALCOHOL | (PC00092); | CH—OH group of donors, NAD or NADP as | biosynthetic process | |
| | DEHYDROGENASE 8 | reductase | acceptor (GO: 0016616) | (GO: 0019438); organic cyclic | |
| | (PTHR42683: SF54) | (PC00198) | | compound biosynthetic | |
| | | | | process (GO: 1901362); | |
| | | | | secondary metabolite | |
| | | | | biosynthetic process | |
| | | | | (GO: 0044550) | |
| AT1G16890 | UBIQUITIN- | | ubiquitin-like protein conjugating enzyme | postreplication | nucleus |
| | CONJUGATING | | activity(GO: 0061650);ubiquitin-protein | repair(GO: 0006301); protein | (GO: 0005634) |
| | ENZYME E2 36 | | transferase activity(GO: 0004842) | K63-linked | |
| | (PTHR24068: SF273) | | | ubiquitination(GO: 0070534) | |
| AT2G30110 | UBIQUITIN- | ligase (PC00142); | ubiquitin-like modifier activating enzyme | cellular response to DNA | cytoplasm |
| | ACTIVATING | transfer/carrier | activity (GO: 0008641) | damage stimulus | (GO: 0005737); |
| | ENZYME E1 1 | protein | | (GO: 0006974); protein | nucleus |
| | (PTHR10953: SF208) | (PC00219) | | ubiquitination (GO: 0016567) | (GO: 0005634) |
| AT4G21100 | DNA DAMAGE- | damaged DNA- | damaged DNA binding (GO: 0003684) | proteasome-mediated | nucleus |
| | BINDING | binding protein | | ubiquitin-dependent protein | (GO: 0005634) |
| | PROTEIN 1 | (PC00086); | | catabolic process | |
| | (PTHR10644: SF3) | mRNA | | (GO: 0043161); protein | |
| | | polyadenylation | | ubiquitination (GO: 0016567) | |
| | | factor (PC00146) | | | |
| AT5G49570 | PEPTIDE-N(4)- | hydrolase | hydrolase activity, acting on carbon- | cellular protein modification | cytosol |
| | (N-ACETYL- | (PC00121) | nitrogen (but not peptide) bonds, in linear | process (GO: 0006464); | (GO: 0005829); |
| | BETA- | | amides (GO: 0016811) | glycoprotein metabolic | nucleus |
| | GLUCOSAMINYL)ASPARAGINE | | | process (GO: 0009100); | (GO: 0005634) |
| | AMIDASE | | | protein quality control for | |
| | (PTHR12143: SF19) | | | misfolded or incompletely | |
| | | | | synthesized proteins | |
| | | | | (GO: 0006515) | |
| AT1G08420 | SERINE/THREONINE- | | | | |
| | PROTEIN | | | | |
| | PHOSPHATASE | | | | |
| | BSL2 | | | | |
| | (PTHR46422: SF7) | | | | |
| AT1G21210 | WALL- | | protein serine/threonine kinase activity | | plasma |
| | ASSOCIATED | | (GO: 0004674) | | membrane(GO: 0005886) |
| | RECEPTOR | | | | |
| | KINASE 2- | | | | |
| | RELATED | | | | |
| | (PTHR27005: SF282) | | | | |
| AT1G48210 | F11A17.22 | | transmembrane receptor protein | | plasma membrane |
| | PROTEIN- | | serine/threonine kinase activity | | (GO: 0005886) |
| | RELATED | | (GO: 0004675) | | |
| | (PTHR27001: SF841) | | | | |
| AT1G51660 | MITOGEN- | | protein serine/threonine kinase activity | activation of protein kinase | cytoplasm |
| | ACTIVATED | | (GO: 0004674) | activity (GO: 0032147); signal | (GO: 0005737) |
| | PROTEIN | | | transduction by protein | |
| | KINASE | | | phosphorylation | |
| | KINASE 4 | | | (GO: 0023014); stress- | |
| | (PTHR24361: SF758) | | | activated protein kinase | |
| | | | | signaling cascade | |
| | | | | (GO: 0031098) | |
| AT2G02560 | CULLIN- | transcription | | protein ubiquitination | nucleus |
| | ASSOCIATED | factor (PC00218) | | (GO: 0016567); protein- | (GO: 0005634) |
| | NEDD8- | | | containing complex assembly | |
| | DISSOCIATED | | | (GO: 0065003) | |
| | PROTEIN 1 | | | | |
| | (PTHR12696: SF0) | | | | |
| AT2G16600 | PEPTIDYL-PROLYL CIS-TRANS | | catalytic activity, acting on a protein | peptidyl-amino acid | Golgi apparatus |
| | ISOMERASE CYP19-1 | | (GO: 0140096); drug binding | modification (GO: 0018193); | (GO: 0005794); |
| | (PTHR11071: SF459) | | (GO: 0008144); peptide binding | protein folding (GO: 006457) | chloroplast |
| | | | (GO: 0042277); unfolded protein binding | | (GO: 0009507); |
| | | | (GO: 0051082) | | cytosol |
| | | | | | (GO: 0005829); |
| | | | | | plasma membrane |
| | | | | | (GO: 0005886); |
| | | | | | vacuole |
| | | | | | (GO: 0005773) |
| AT2G37710 | L-TYPE LECTIN- | | transmembrane receptor protein | defense response to bacterium | plasma membrane |
| | DOMAIN | | serine/threonine kinase activity | (GO: 0042742) | (GO: 0005886) |
| | CONTAINING | | (GO: 0004675) | | |
| | RECEPTOR | | | | |
| | KINASE IV.1 | | | | |
| | (PTHR27007: SF207) | | | | |
| AT3G09350 | HSP70-BINDING | | ATPase activity (GO: 0016887); | | endoplasmic |
| | PROTEIN 1 | | nucleoside-triphosphatase regulator | | reticulum |
| | (PTHR19316: SF18) | | activity (GO: 0060589); purine nucleotide | | (GO: 0005783); |
| | | | binding (GO: 0017076) | | plasma membrane |
| | | | | | (GO: 0005886); |
| | | | | | vacuole |
| | | | | | (GO: 0005773) |
| AT3G12580 | HEAT SHOCK | | ATP binding (GO: 0005524); ATPase | cellular response to heat | cytoplasm |
| | 70 KDA | | activity, coupled (GO: 0042623); heat | (GO: 0034605); cellular | (GO: 0005737) |
| | PROTEIN | | shock protein binding (GO: 0031072); | response to unfolded protein | |
| | COGNATE 1- | | unfolded protein binding (GO: 0051082) | (GO: 0034620); chaperone- | |
| | RELATED | | | mediated protein folding | |
| | (PTHR19375: SF395) | | | (GO: 0061077) | |
| AT3G14840 | SUBFAMILY | | protein serine/threonine kinase activity | protein autophosphorylation | plasma membrane |
| | NOT NAMED | | (GO: 0004674) | (GO: 0046777) | (GO: 0005886) |
| | (PTHR27006: SF136) | | | | |
| AT3G19420 | PHOSPHATIDYLINOSITOL 3,4,5- | protein | phosphoprotein phosphatase activity | cell cycle (GO: 0007049); | |
| | TRISPHOSPHATE 3- | phosphatase | (GO: 0004721) | phospholipid metabolic | |
| | PHOSPHATASE | (PC00195) | | process (GO: 0006644); | |
| | TPTE2- | | | protein phosphorylation | |
| | RELATED | | | (GO: 0006468) | |
| | (PTHR12305: SF32) | | | | |
| AT3G48990 | OXALATE-- | dehydrogenase | ligase activity (GO: 0016874); | cellular amino acid | |
| | COA LIGASE | (PC00092); ligase | oxidoreductase activity (GO: 0016491) | biosynthetic process | |
| | (PTHR43201: SF14) | (PC00142) | | (GO: 0008652); coenzyme | |
| | | | | metabolic process | |
| | | | | (GO: 0006732); fatty acid | |
| | | | | metabolic process | |
| | | | | (GO: 0006631) | |
| AT4G15802 | HEAT SHOCK | | | cellular response to heat | cytosol |
| | FACTOR | | | (GO: 0034605) | (GO: 0005829); |
| | BINDING | | | | nucleus |
| | PROTEIN | | | | (GO: 0005634) |
| | (PTHR19424: SF0) | | | | |
| AT4G26070 | MITOGEN- | | protein serine/threonine kinase activity | activation of protein kinase | cytoplasm |
| | ACTIVATED | | (GO: 0004674) | activity (GO: 0032147); signal | (GO: 0005737) |
| | PROTEIN | | | transduction by protein | |
| | KINASE | | | phosphorylation | |
| | KINASE 1 | | | (GO: 0023014); stress- | |
| | (PTHR24361: SF735) | | | activated protein kinase | |
| | | | | signaling cascade | |
| | | | | (GO: 0031098) | |
| AT4G26120 | REGULATORY | | | defense response to bacterium | cytoplasm |
| | PROTEIN NPR2 | | | (GO: 0042742); jasmonic acid | (GO: 0005737); |
| | (PTHR46475: SF5) | | | mediated signaling pathway | nucleus |
| | | | | (GO: 0009867) regulation of | (GO: 0005634) |
| | | | | signal transduction | |
| | | | | (GO: 0009966) response to | |
| | | | | fungus (GO: 0009620) | |
| AT4G39090 | CYSTEINE | cysteine protease | cysteine-type endopeptidase activity | proteolysis involved in | extracellular space |
| | PROTEASE | (PC00081); | (GO: 0004197) | cellular protein catabolic | (GO: 0005615); |
| | RD19A | protease inhibitor | | process (GO: 0051603) | lysosome |
| | (PTHR12411: SF611) | (PC00191) | | | (GO: 0005764) |
| AT5G02490 | HEAT SHOCK | | ATP binding (GO: 0005524); ATPase | cellular response to heat | cytoplasm |
| | 70 KDA | | activity, coupled (GO: 0042623); heat | (GO: 0034605); cellular | (GO: 0005737) |
| | PROTEIN | | shock protein binding (GO: 0031072); | response to unfolded protein | |
| | COGNATE 1- | | unfolded protein binding (GO: 0051082) | (GO: 0034620); chaperone- | |
| | RELATED | | | mediated protein folding | |
| | (PTHR19375: SF395) | | | (GO: 0061077) | |
| AT5G14250 | COP9 | enzyme | | protein ubiquitination | COP9 signalosome |
| | SIGNALOSOME | modulator | | (GO: 0016567); ubiquitin- | (GO: 0008180) |
| | COMPLEX | (PC00095) | | dependent protein catabolic | |
| | SUBUNIT 3 | | | process (GO: 0006511) | |
| | (PTHR10758: SF1) | | | | |
| AT5G43060 | CYSTEINE | cysteine protease | cysteine-type endopeptidase activity | proteolysis involved in | extracellular space |
| | PROTEASE | (PC00081); | (GO: 0004197) | cellular protein catabolic | (GO: 0005615); |
| | RD21B- | protease inhibitor | | process (GO: 0051603) | lysosome |
| | RELATED | (PC00191) | | | (GO: 0005764) |
| | (PTHR12411: SF682) | | | | |
| AT5G45110 | REGULATORY | | | defense response to bacterium | nucleus |
| | PROTEIN NPR3 | | | (GO: 0042742); jasmonic acid | (GO: 0005634) |
| | (PTHR46475: SF8) | | | mediated signaling pathway | |
| | | | | (GO: 0009867); regulation of | |
| | | | | signal transduction | |
| | | | | (GO: 0009966); response to | |
| | | | | fungus (GO: 0009620) | |
| AT5G35530 | 40S | ribosomal protein | catalytic activity, acting on DNA | DNA repair (GO: 0006281); | cytosolic small ribosomal |
| | RIBOSOMAL | (PC00202) | (GO: 0140097); damaged DNA binding | positive regulation of | subunit (GO: 0022627); |
| | PROTEIN S3 | | (GO: 0003684); hydrolase activity | macromolecule metabolic | nucleus (GO: 0005634) |
| | (PTHR11760: SF32) | | (GO: 0016787); structural constituent of | process (GO: 0010604); | |
| | | | ribosome (GO: 0003735) | positive regulation of | |
| | | | | nucleobase-containing | |
| | | | | compound metabolic process | |
| | | | | (GO: 0045935); positive | |
| | | | | regulation of response to | |
| | | | | DNA damage stimulus | |
| | | | | (GO: 2001022); regulation of | |
| | | | | DNA repair (GO: 0006282) | |
| AT2G29570 | PROLIFERATING | DNA polymerase | catalytic activity (GO: 0003824); enzyme | DNA biosynthetic process | intracellular part |
| | CELL | processivity factor | regulator activity (GO: 0030234) | (GO: 0071897); DNA strand | (GO: 0044424); |
| | NUCLEAR | (PC00015) | | elongation involved in DNA | protein-containing |
| | ANTIGEN | | | replication (GO: 0006271); | complex |
| | (PTHR11352: SF0) | | | RNA biosynthetic process | (GO: 0032991) |
| | | | | (GO: 0032774); RNA | |
| | | | | catabolic process | |
| | | | | (GO: 0006401) | |
| AT3G06010 | ATP- | DNA helicase | | | |
| | DEPENDENT | (PC00011) | | | |
| | HELICASE BRM | | | | |
| | (PTHR10799: SF854) | | | | |
| AT3G07930 | METHYL-CPG- | methyl- | DNA binding (GO: 0003677) | response to radiation | |
| | BINDING | transferase | | (GO: 0009314) | |
| | DOMAIN | (PC00155); | | | |
| | PROTEIN 4- | nuclease | | | |
| | LIKE PROTEIN | (PC00170) | | | |
| | (PTHR15074: SF0) | | | | |
| AT3G14890 | BIFUNCTIONAL | | double-stranded DNA binding | DNA repair (GO: 0006281); | |
| | POLYNUCLEOTIDE | | (GO: 0003690); kinase activity | nucleoside monophosphate | |
| | PHOSPHATASE/ | | (GO: 0016301); phosphatase activity | metabolic process | |
| | KINASE | | (GO: 0016791); phosphotransferase | (GO: 0009123); nucleotide | |
| | (PTHR12083: SF9) | | activity, alcohol group as acceptor | biosynthetic process | |
| | | | (GO: 0016773); phosphotransferase | (GO: 0009165); nucleotide | |
| | | | activity, phosphate group as acceptor | phosphorylation | |
| | | | (GO: 0016776) | (GO: 0046939) | |
| AT4G13350 | SUBFAMILY | | | | |
| | NOT NAMED | | | | |
| | (PTHR46085: SF3) | | | | |
| AT1G01960 | EXCHANGE | | | | |
| | FACTOR FOR | | | | |
| | ARF 6, | | | | |
| | ISOFORM H | | | | |
| | (PTHR10663: SF328) | | | | |
| AT4G02510 | TRANSLOCASE | | | | |
| | OF CHLOROPLAST | | | | |
| | 159, | | | | |
| | CHLOROPLASTIC | | | | |
| | (PTHR10903: SF120) | | | | |
| AT2G34480 | 60S | | | | cytosolic large |
| | RIBOSOMAL | | | | ribosomal subunit |
| | PROTEIN L18A-1- | | | | (GO: 0022625) |
| | RELATED | | | | |
| | (PTHR10052: SF39) | | | | |
| AT1G02450 | PROTEIN NIM1- | | | | |
| | INTERACTING 1 | | | | |
| | (PTHR33669: SF11) | | | | |
| AT1G55450 | S-ADENOSYL- | | | | |
| | L-METHIONINE- | | | | |
| | DEPENDENT | | | | |
| | METHYLTRANSFERASES | | | | |
| | SUPERFAMILY | | | | |
| | PROTEIN | | | | |
| | (PTHR44575: SF6) | | | | |
| AT1G60140 | ALPHA,ALPHA- | | | | |
| | TREHALOSE- | | | | |
| | PHOSPHATE | | | | |
| | SYNTHASE | | | | |
| | [UDP- | | | | |
| | FORMING] 10- | | | | |
| | RELATED | | | | |
| | (PTHR10788: SF81) | | | | |
| AT1G66090 | | | | | |
| AT1G72910 | DISEASE | | | | |
| | RESISTANCE | | | | |
| | PROTEIN (TTR- | | | | |
| | NBS CLASS)- | | | | |
| | RELATED | | | | |
| | (PTHR11017: SF207) | | | | |
| AT2G03440 | NODULIN- | | | | |
| | RELATED | | | | |
| | PROTEIN 1 | | | | |
| | (PTHR35098: SF4) | | | | |
| AT2G14560 | PROTEIN LURP- | | | | |
| | ONE-RELATED | | | | |
| | 1-RELATED | | | | |
| | (PTHR31087: SF58) | | | | |
| AT2G14610 | PATHOGENESIS- | defense/ | | | extracellular space |
| | RELATED | immunity protein | | | (GO: 0005615) |
| | PROTEIN 1 | (PC00090) | | | |
| | (PTHR10334: SF406) | | | | |
| AT2G17265 | HOMOSERINE | | | | |
| | KINASE | | | | |
| | (PTHR20861: SF1) | | | | |
| AT2G24850 | AMINOTRANSFERASE | | | | |
| | TAT3- | | | | |
| | RELATED | | | | |
| | (PTHR45744: SF21) | | | | |
| AT2G26560 | PATATIN-LKE | | | | |
| | PROTEIN 2 | | | | |
| | (PTHR32176: SF0) | | | | |
| AT2G37970 | SOUL HEME- | | | | |
| | BINDING | | | | |
| | FAMILY | | | | |
| | PROTEIN | | | | |
| | (PTHR11220: SF58) | | | | |
| AT3G01290 | HYPERSENSITIVE- | cytoskeletal | structural molecule activity (GO: 0005198) | | cytoskeleton |
| | INDUCED | protein | | | (GO: 0005856) |
| | RESPONSE | (PC00085); | | | |
| | PROTEIN 3 | protease inhibitor | | | |
| | (PTHR43327: SF19) | (PC00191) | | | |
| AT3G04210 | ATP BINDING | | | | |
| | PROTEIN- | | | | |
| | RELATED | | | | |
| | (PTHR11017: SF297) | | | | |
| AT3G18520 | HISTONE | | | chromatin organization | |
| | DEACETYLASE | | | (GO: 0006325) | |
| | (PTHR45634: SF11) | | | | |
| AT3G43810 | CALMODULIN- | calmodulin | calcium ion binding (GO: 0005509) | calcium-mediated signaling | intracellular |
| | 7-RELATED | (PC00061) | | (GO: 0019722) | (GO: 0005622) |
| | (PTHR23050: SF385) | | | | |
| AT3G44480 | ATP BINDING | | | | |
| | PROTEIN- | | | | |
| | RELATED | | | | |
| | (PTHR11017: SF297) | | | | |
| AT3G48090 | PROTEIN EDS1- | | | | |
| | RELATED | | | | |
| | (PTHR47090: SF2) | | | | |
| AT3G50930 | PROTEIN | | | | |
| | HYPER- | | | | |
| | SENSITIVITY- | | | | |
| | RELATED 4 | | | | |
| | (PTHR23070: SF81) | | | | |
| AT3G51250 | SUBFAMILY | | | | plasma membrane |
| | NOT NAMED | | | | (GO: 0005886) |
| | (PTHR21068: SF35) | | | | |
| AT3G52430 | LIPASE-LIKE | | | | |
| | PAD4 | | | | |
| | (PTHR47413: SF2) | | | | |
| AT4G16950 | DISEASE | | | | |
| | RESISTANCE | | | | |
| | PROTEIN (TIR- | | | | |
| | NBS-LRR | | | | |
| | CLASS)- | | | | |
| | RELATED | | | | |
| | (PTHR11017: SF274) | | | | |
| AT4G19510 | DISEASE | | | | |
| | RESISTANCE | | | | |
| | PROTEIN (TIR- | | | | |
| | NBS-LRR | | | | |
| | CLASS) | | | | |
| | (PTHR11017: SF293) | | | | |
| AT4G33050 | IQ DOMAIN- | | | | |
| | CONTAINING | | | | |
| | PROTEIN IQM1 | | | | |
| | (PTHR31250: SF33) | | | | |
| AT5G41750 | DISEASE | | | | |
| | RESISTANCE | | | | |
| | PROTEIN (TIR- | | | | |
| | NBS-LRR | | | | |
| | CLASS) | | | | |
| | FAMILY | | | | |
| | (PTHR11017: SF291) | | | | |
| AT5G45510 | DISEASE | | | | |
| | RESISTANCE | | | | |
| | PROTEIN (TIR- | | | | |
| | NBS-LRR | | | | |
| | CLASS)- | | | | |
| | RELATED | | | | |
| | (PTHR11017: SF212) | | | | |
| AT5G45520 | LEUCINE-RICH | | | | |
| | REPEAT (LRR) | | | | |
| | FAMILY | | | | |
| | PROTEIN- | | | | |
| | RELATED | | | | |
| | (PTHR23155: SF1076) | | | | |
| AT5G48620 | DISEASE | | | | |
| | RESISTANCE | | | | |
| | RPP8-LIKE | | | | |
| | PROTEIN 3- | | | | |
| | RELATED | | | | |
| | (PTHR23155: SF933) | | | | |
| AT5G54310 | ADP- | | | | |
| | RIBOSYLATION | | | | |
| | FACTOR | | | | |
| | GTPASE- | | | | |
| | ACTIVATING | | | | |
| | PROTEIN | | | | |
| | AGD5- | | | | |
| | RELATED | | | | |
| | (PTHR46419: SF2) | | | | |
| AT5G57560 | XYLOGLUCAN | | | | |
| | ENDOTRANSGLUCOSYLASE/ | | | | |
| | HYDROLASE | | | | |
| | PROTEIN 22 | | | | |
| | (PTHR31062: SF188) | | | | |
| AT5G45500 | LEUCINE-RICH | | | | |
| | REPEAT (LRR) | | | | |
| | FAMILY | | | | |
| | PROTEIN- | | | | |
| | RELATED | | | | |
| | (PTHR23155: SF1076) | | | | |
| AT1G68730 | F24J5.3 | | chaperone binding (GO: 0051087) | protein folding (GO: 0006457); | mitochondrion |
| | (PTHR20922: SF19) | | | protein import into | (GO: 0005739) |
| | | | | mitochondrial matrix | |
| | | | | (GO: 0030150); protein | |
| | | | | stabilization (GO: 0050821) | |
| AT1G68200 | ZINC FINGER | RNA binding | | | |
| | CCCH DOMAIN- | protein | | | |
| | CONTAINING | (PC00031) | | | |
| | PROTEIN 15 | | | | |
| | (PTHR12547: SF126) | | | | |
| AT2G21790 | RIBONUCLEOSIDE- | reductase | oxidoreductase activity (GO: 0016491) | carbohydrate derivative | cytosolic part |
| | DIPHOSPHATE | (PC00198) | | biosynthetic process | (GO: 0044445); |
| | REDUCTASE | | | (GO: 1901137); nucleotide | oxidoreductase |
| | LARGE | | | biosynthetic process | complex |
| | SUBUNIT | | | (GO: 0009165) | (GO: 1990204) |
| | (PTHR11573: SF6) | | | | |
| AT1G05720 | SELENOPROTEIN F | | | | |
| | (PTHR13077: SF6) | | | | |
| AT1G11680 | LANOSTEROL | oxygenase | | oxidation-reduction process | |
| | 14-ALPHA | (PC00177) | | (GO: 0055114); sterol | |
| | DEMETHYLASE | | | metabolic process | |
| | (PTHR24286: SF24) | | | (GO: 0016125) | |
| AT1G16350 | INOSINE-5′- | | oxidoreductase activity, acting on the | nucleobase-containing small | |
| | MONOPHOSPHATE | | CH—OH group of donors, NAD or NADP as | molecule biosynthetic process | |
| | DEHYDROGENASE | | acceptor (GO: 0016616) | (GO: 0034404); purine | |
| | (PTHR11911: SF111) | | | ribonucleoside triphosphate | |
| | | | | biosynthetic process | |
| | | | | (GO: 0009206); purine | |
| | | | | ribonucleotide biosynthetic | |
| | | | | process (GO: 0009152) | |
| AT1G22400 | UDP- | | UDP-glucosyltransferase activity | | intracellular |
| | GLYCOSYLTRANSFERASE | | (GO: 0035251) | | membrane- |
| | 85A1-RELATED | | | | bounded |
| | (PTHR11926: SF928) | | | | organelle |
| | | | | | (GO: 0043231) |
| AT1G48320 | 1,4-DIHYDROXY-2- | | hydrolase activity, acting on ester bonds | | cytosol |
| | NAPHTHOYL- | | (GO: 0016788) | | (GO: 0005829) |
| | COA | | | | |
| | THIOESTERASE 1 | | | | |
| | (PTHR43240: SF5) | | | | |
| AT1G70580 | GLUTAMATE- | transaminase | transferase activity (GO: 0016740) | cellular amino acid metabolic | |
| | GLYOXYLATE | (PC00216) | | process (GO: 0006520); | |
| | AMINOTRANSFERASE 2 | | | porphyrin-containing | |
| | (PTHR11751: SF373) | | | compound metabolic process | |
| | | | | (GO: 0006778) | |
| AT2G01490 | PHYTANOYL- | | | | |
| | COA | | | | |
| | DIOXYGENASE | | | | |
| | DOMAIN- | | | | |
| | CONTAINING | | | | |
| | PROTEIN 1 | | | | |
| | (PTHR20883: SF15) | | | | |
| AT2G26400 | 1,2- | oxidoreductase | dioxygenase activity (GO: 0051213) | cellular amino acid metabolic | |
| | DIHYDROXY-3- | (PC00176) | | process (GO: 0006520); sulfur | |
| | KETO-5- | | | compound metabolic process | |
| | METHYLTHIOPENTENE | | | (GO: 0006790) | |
| | DIOXYGENASE | | | | |
| | (PTHR23418: SF0) | | | | |
| AT2G29320 | SUBFAMILY | dehydrogenase | oxidoreductase activity (GO: 0016491) | steroid metabolic process | |
| | NOT NAMED | (PC00092); | | (GO: 0008202); transport | |
| | (PTHR42898: SF33) | reductase | | (GO: 0006810) | |
| | | (PC00198) | | | |
| AT2G43820 | UDP- | | UDP-glucosyltransferase activity | | intracellular |
| | GLYCOSYLTRANSFERASE | | (GO: 0035251) | | membrane- |
| | 74F2 | | | | bounded |
| | (PTHR11926: SF1147) | | | | organelle |
| | | | | | (GO: 0043231) |
| AT4G13180 | SUBFAMILY | dehydrogenase | oxidoreductase activity (GO: 0016491) | steroid metabolic process | |
| | NOT NAMED | (PC00092); | | (GO: 0008202) | |
| | (PTHR43361: SF1) | reductase | | | |
| | | (PC00198) | | | |
| AT4G22220 | IRON-SULFUR | | cofactor binding (GO: 0048037); iron ion | cellular iron ion homeostasis | cytoplasm |
| | CLUSTER | | binding (GO: 0005506) | (GO: 0006879) | (GO: 0005737) |
| | ASSEMBLY | | | | |
| | ENZYME ISCU, | | | | |
| | MITOCHONDRIAL | | | | |
| | (PTHR10093: SF8) | | | | |
| AT5G14240 | VIRAL IAP- | | | | |
| | ASSOCIATED | | | | |
| | FACTOR | | | | |
| | HOMOLOG | | | | |
| | (PTHR45809: SF3) | | | | |
| AT5G22140 | FAD/NAD(P)− | dehydrogenase | electron transfer activity (GO: 0009055); | respiratory electron transport | cytoplasm |
| | BINDING | (PC00092); | flavin adenine dinucleotide binding | chain (GO: 0022904) | (GO: 0005737) |
| | OXIDOREDUCTASE | oxidase | (GO: 0050660) | | |
| | FAMILY | (PC00175); | | | |
| | PROTEIN | reductase | | | |
| | (PTHR43735: SF14) | (PC00198) | | | |
| AT5G40760 | GLUCOSE-6- | dehydrogenase | oxidoreductase activity (GO: 0016491) | monosaccharide metabolic | |
| | PHOSPHATE 1- | (PC00092) | | process (GO: 0005996) | |
| | DEHYDROGENASE | | | | |
| | (PTHR23429: SF0) | | | | |
| AT5G48180 | NITRILE- | | catalytic activity (GO: 0003824); enzyme | cellular nitrogen compound | Cytosol |
| | SPECIFIER | | regulator activity (GO: 0030234) | biosynthetic process | (GO: 0005829); |
| | PROTEIN 5 | | | (GO: 0044271); | nucleus |
| | (PTHR47435: SF3) | | | organonitrogen compound | (GO: 0005634) |
| | | | | biosynthetic process | |
| | | | | (GO: 1901566) | |
| AT5G47040 | LON PROTEASE | | | | |
| | HOMOLOG 2, | | | | |
| | PEROXISOMAL | | | | |
| | (PTHR10046: SF24) | | | | |
| AT3G18060 | WD REPEAT- | non-motor actin | actin filament binding (GO: 0051015) | actin filament | actin filament |
| | CONTAINING | binding protein | | depolymerization | (GO: 0005884); |
| | PROTEIN 1 | (PC00165) | | (GO: 0030042); positive | cortical actin |
| | (PTHR19856: SF0) | | | regulation of cytoskeleton | cytoskeleton |
| | | | | organization (GO: 0051495); | (GO: 0030864) |
| | | | | positive regulation of | |
| | | | | supramolecular fiber | |
| | | | | organization (GO: 1902905); | |
| | | | | regulation of actin | |
| | | | | polymerization or | |
| | | | | depolymerization | |
| | | | | (GO: 0008064) | |
| AT2G31200 | ACTIN- | non-motor actin | actin binding (GO: 0003779); structural | cellular component | actin cytoskeleton |
| | DEPOLYMERIZING | binding protein | molecule activity (GO: 0005198) | morphogenesis | (GO: 0015629) |
| | FACTOR 6 | (PC00165) | | (GO: 0032989); protein | |
| | (PTHR11913: SF84) | | | metabolic process | |
| | | | | (GO: 0019538) | |
| AT1G04860 | UBIQUITIN | | cysteine-type endopeptidase activity | protein deubiquitination | |
| | SPECIFIC | | (GO: 0004197); thiol-dependent ubiquitin- | (GO: 0016579) | |
| | PEPTIDASE 45 | | specific protease activity (GO: 0004843) | | |
| | (PTHR24006: SF661) | | | | |
| AT1G08050 | T6D22.13 | ion channel | | | |
| | (PTHR10579: SF131) | (PC00133); | | | |
| | | metalloprotease | | | |
| | | (PC00153) | | | |
| AT2G04430 | MUTT/NUDIX | nucleotide | cofactor binding (GO: 0048037); | | |
| | FAMILY | phosphatase | nucleotide binding (GO: 0000166); | | |
| | PROTEIN- | (PC00173) | nucleotide diphosphatase activity | | |
| | RELATED | | (GO: 0004551) | | |
| | (PTHR13994: SF26) | | | | |
| AT2G20190 | CLIP- | structural protein | structural molecule activity (GO: 0005198) | cell cycle (GO: 0007049) | |
| | ASSOCIATED | (PC00211) | | | |
| | PROTEIN | | | | |
| | (PTHR21567: SF67) | | | | |
| AT2G21470 | SUMO- | ligase (PC00142); | ubiquitin-like modifier activating enzyme | protein sumoylation | catalytic complex |
| | ACTIVATING | transfer/carrier | activity (GO: 0008641) | (GO: 0016925) | (GO: 1902494); |
| | ENZYME | protein | | | cytoplasm |
| | SUBUNIT 2 | (PC00219) | | | (GO: 0005737); |
| | (PTHR10953: SF5) | | | | nuclear part |
| | | | | | (GO: 0044428) |
| AT2G28450 | TRNA (URACIL-5-)- | | | | |
| | METHYLTRANSFERASE | | | | |
| | HOMOLOG A | | | | |
| | (PTHR45904: SF2) | | | | |
| AT2G32160 | CARNOSINE N- | | S-adenosylmethionine-dependent | | |
| | METHYLTRANSFERASE | | methyltransferase activity (GO: 0008757) | | |
| | (PTHR12303: SF6) | | | | |
| AT2G47110 | UBIQUITIN-40S | ribosomal protein | ubiquitin protein ligase binding | modification-dependent | Cytoplasm |
| | RIBOSOMAL | (PC00202) | (GO: 0031625) | protein catabolic process | (GO: 0005737); |
| | PROTEIN S27A-2 | | | (GO: 0019941); protein | nucleus |
| | (PTHR10666: SF339) | | | ubiquitination (GO: 0016567) | (GO: 0005634) |
| AT3G07990 | SERINE | serine protease | peptidase activity, acting on L-amino acid | proteolysis involved in | |
| | CARBOXYPEPTIDASE- | (PC00203) | peptides (GO: 0070011); serine hydrolase | cellular protein catabolic | |
| | LIKE 27 | | activity (GO: 0017171) | process (GO: 0051603) | |
| | (PTHR11802: SF198) | | | | |
| AT3G20630 | UBIQUITINYL | | cysteine-type endopeptidase activity | protein deubiquitination | |
| | HYDROLASE 1 | | (GO: 0004197); thiol-dependent ubiquitin- | (GO: 0016579) | |
| | (PTHR24006: SF700) | | specific protease activity (GO: 0004843) | | |
| AT3G23570 | PROTEIN AIM2 | | | | |
| | (PTHR17630: SF44) | | | | |
| AT3G50590 | TRANSDUCIN/ | | Atg12 activating enzyme activity | Atg1/ULK1 kinase complex | Autophagosome |
| | WD40 REPEAT- | | (GO: 0019778); Atg12 conjugating | assembly (GO: 1904745); | (GO: 0005776); |
| | LIKE | | enzyme activity (GO: 0061651); Atg12 | autophagosome assembly | vacuolar |
| | SUPERFAMILY | | ligase activity (GO: 0061660); Atg8- | (GO: 0000045); | membrane |
| | PROTEIN | | specific protease activity (GO: 0019786) | transmembrane transport | (GO: 0005774) |
| | (PTHR19878: SF17) | | | (GO: 0055085) | |
| AT3G58040 | E3 UBIQUITIN- | ubiquitin-protein | ubiquitin protein ligase activity | | cytoplasm |
| | PROTEIN | ligase (PC00234) | (GO: 0061630) | | (GO: 0005737) |
| | LIGASE SINAT2 | | | | |
| | (PTHR10315: SF79) | | | | |
| AT4G01870 | TOLB PROTEIN- | | | | |
| | LIKE PROTEIN | | | | |
| | (PTHR32161: SF9) | | | | |
| AT4G17830 | ACETYLORNITHINE | deacetylase | | | |
| | DEACETYLASE | (PC00087); | | | |
| | (PTHR43808: SF3) | metalloprotease | | | |
| | | (PC00153) | | | |
| AT4G30890 | UBIQUITIN | cysteine protease | cysteine-type endopeptidase activity | protein deubiquitination | |
| | CARBOXYL- | (PC00081) | (GO: 0004197); thiol-dependent ubiquitin- | (GO: 0016579) | |
| | TERMINAL | | specific protease activity (GO: 0004843) | | |
| | HYDROLASE 10 | | | | |
| | (PTHR24006: SF687) | | | | |
| AT5G60360 | PRO- | cysteine protease) | cysteine-type endopeptidase activity | proteolysis involved in | extracellular space |
| | CATHEPSIN H | PC00081); | (GO: 0004197) | cellular protein catabolic | (GO: 0005615); |
| | (PTHR12411: SF642) | protease inhibitor | | process (GO: 0051603) | lysosome |
| | | (PC00191) | | | (GO: 0005764) |
| AT5G60510 | DEHYDRODOLICHYL | acyltransferase | transferase activity, transferring alkyl or | alcohol metabolic process | endoplasmic |
| | DIPHOSPHATE | (PC00042) | aryl (other than methyl) groups | (GO: 0006066); isoprenoid | reticulum |
| | SYNTHASE | | (GO: 0016765) | biosynthetic process | (GO: 0005783); |
| | COMPLEX | | | (GO: 0008299); organic | plasma membrane |
| | SUBUNIT | | | hydroxy compound | (GO: 0005886); |
| | DHDDS | | | biosynthetic process | vacuole |
| | (PTHR10291: SF29) | | | (GO: 1901617); small | (GO: 0005773) |
| | | | | molecule biosynthetic process | |
| | | | | (GO: 0044283) | |
| AT5G61790 | CALNEXIN 14D- | calcium-binding | calcium ion binding (GO: 0005509) | Exocytosis (GO: 0006887); | |
| | RELATED | protein | | intracellular protein transport | |
| | (PTHR11073: SF1) | (PC00060); | | (GO: 0006886); protein | |
| | | chaperone | | folding (GO: 0006457); | |
| | | (PC00072) | | protein metabolic process | |
| | | | | (GO: 0019538) | |
| AT5G67340 | U-BOX | cell adhesion | | | cytoplasm |
| | DOMAIN- | molecule | | | (GO: 0005737); |
| | CONTAINING | (PC00069); | | | nucleus |
| | PROTEIN 2 | cytoskeletal | | | (GO: 0005634) |
| | (PTHR23315: SF263) | protein | | | |
| | | (PC00085); | | | |
| | | signaling | | | |
| | | molecule | | | |
| | | (PC00207); | | | |
| | | storage protein | | | |
| | | (PC00210) | | | |
| AT3G15610 | SERINE- | | | | |
| | THREONINE | | | | |
| | KINASE | | | | |
| | RECEPTOR- | | | | |
| | ASSOCIATED | | | | |
| | PROTEIN | | | | |
| | (PTHR44156: SF3) | | | | |
| AT3G45620 | DDB1 AND | G-protein | GTPase activity (GO: 0003924); protein | nucleobase-containing | |
| | CUL4- | (PC00020) | binding (GO: 0005515) | compound metabolic process | |
| | ASSOCIATED | | | (GO: 0006139); regulation of | |
| | FACTOR 8 | | | nucleobase-containing | |
| | (PTHR15574: SF21) | | | compound metabolic process | |
| | | | | (GO: 0019219) | |
| AT5G24710 | TRANSDUCIN/ | | | | |
| | WD40 REPEAT- | | | | |
| | LIKE | | | | |
| | SUPERFAMILY | | | | |
| | PROTEIN | | | | |
| | (PTHR45521: SF2) | | | | |
| AT3G18580 | NUCLEIC ACID- | DNA binding | helicase activity (GO: 0004386); single- | DNA biosynthetic process | intracellular non-membrane- |
| | BINDING, OB- | protein | stranded DNA binding (GO: 0003697) | (GO: 0071897); DNA- | bounded organelle |
| | FOLD-LIKE | (PC00009) | | dependent DNA replication | (GO: 0043232); |
| | PROTEIN | | | (GO: 0006261); mitochondrial | mitochondrial |
| | (PTHR10302: SF16) | | | DNA metabolic process | matrix |
| | | | | (GO: 0032042); positive | (GO: 0005759) |
| | | | | regulation of hydrolase | |
| | | | | activity (GO: 0051345) | |
| AT4G31210 | DNA | enzyme | catalytic activity (GO: 0003824); nucleic | DNA replication | |
| | TOPOISOMERASE, | modulator | acid binding (GO: 0003676); protein | (GO: 0006260); cell cycle | |
| | TYPE IA, | (PC00095); | binding (GO: 0005515) | (GO: 0007049) | |
| | CORE | isomerase | | | |
| | (PTHR42785: SF1) | (PC00135); | | | |
| | | nucleic acid | | | |
| | | binding | | | |
| | | (PC00171) | | | |
| AT2G41740 | VILLIN-2 | | | | |
| | (PTHR11977: SF85) | | | | |
| AT3G19960 | MYOSIN-1- | G-protein | enzyme regulator activity (GO: 0030234); | cellular component | actin cytoskeleton |
| | RELATED | modulator | motor activity (GO: 0003774); protein | morphogenesis | (GO: 0015629); |
| | (PTHR13140: SF780) | (PC00022); actin | binding (GO: 0005515); structural | (GO: 0032989); cytokinesis | cell junction |
| | | binding motor | molecule activity(GO: 0005198) | (GO: 0000910); intracellular | (GO: 0030054); |
| | | protein | | protein transport | plasma membrane |
| | | (PC00040); cell | | (GO: 0006886); intracellular | (GO: 0005886) |
| | | junction protein | | signal transduction | |
| | | (PC00070) | | (GO: 0035556); movement of | |
| | | | | cell or subcellular component | |
| | | | | (GO: 0006928); vesicle- | |
| | | | | mediated transport | |
| | | | | (GO: 0016192) | |
| AT1G27970 | NUCLEAR | | | nucleocytoplasmic transport | nuclear pore |
| | TRANSPORT | | | (GO: 0006913) | central transport |
| | FACTOR 2B | | | | channel |
| | (PTHR12612: SF31) | | | | (GO: 0044613); |
| | | | | | plasma membrane |
| | | | | | (GO: 0005886); |
| | | | | | vacuole |
| | | | | | (GO: 0005773) |
| AT3G06720 | IMPORTIN | | nuclear localization sequence binding | NLS-bearing protein import | cytosol |
| | SUBUNIT | | (GO: 0008139); protein transporter activity | into nucleus (GO: 006607) | (GO: 0005829); |
| | ALPHA- | | (GO: 0008565) | | nuclear pore |
| | RELATED | | | | (GO: 0005643); |
| | (PTHR23316: SF70) | | | | nucleoplasm |
| | | | | | (GO: 0005654); |
| | | | | | plasma membrane |
| | | | | | (GO: 0005886); |
| | | | | | vacuole |
| | | | | | (GO: 0005773) |
| AT1G59610 | DYNAMIN-2B | hydrolase | GTPase activity (GO: 0003924); | mitochondrial fission | cytoplasm |
| | (PTHR11566: SF158) | (PC00121); | microtubule binding (GO: 0008017) | (GO: 0000266) | (GO: 0005737) |
| | | microtubule | | | |
| | | family | | | |
| | | cytoskeletal | | | |
| | | protein | | | |
| | | (PC00157); small | | | |
| | | GTPase | | | |
| | | (PC00208) | | | |
| AT3G13870 | PROTEIN SEY1 | | GTPase activity (GO: 0003924) | endoplasmic reticulum | endoplasmic |
| | (PTHR45923: SF2) | | | organization (GO: 0007029); | reticulum |
| | | | | organelle membrane fusion | (GO: 0005783); |
| | | | | (GO: 0090174) | plasma membrane |
| | | | | | (GO: 0005886); |
| | | | | | vacuole |
| | | | | | (GO: 0005773) |
| AT5G24520 | PROTEIN | | | | Nucleus |
| | TRANSPARENT | | | | (GO: 0005634) |
| | TESTA | | | | |
| | GLABRA 1 | | | | |
| | (PTHR19919: SF10) | | | | |
| AT1G07140 | RAN-BINDING | G-protein | GTPase activator activity (GO: 0005096); | | nuclear pore |
| | PROTEIN 1 | modulator | GTPase activity (GO: 0003924); Ras | | (GO: 0005643); |
| | HOMOLOG A | (PC00022) | GTPase binding (GO: 0017016) | | plasma membrane |
| | (PTHR23138: SF133) | | | | (GO: 0005886); |
| | | | | | vacuole |
| | | | | | (GO: 0005773) |
| AT3G19870 | AP-5 COMPLEX | | | endosomal transport | cytoplasmic part |
| | SUBUNIT | | | (GO: 0016197) | (GO: 0044444); |
| | BETA-1 | | | | membrane protein |
| | (PTHR34033: SF1) | | | | complex |
| | | | | | (GO: 0098796) |
| AT3G28710 | V-TYPE | ATP synthase | ATPase activity, coupled to | vacuolar acidification | lysosomal |
| | PROTON | (PC00002); | transmembrane movement of substances | (GO: 0007035); vacuolar | membrane |
| | ATPASE | hydrolase | (GO: 0042626); proton transmembrane | transport (GO: 0007034) | (GO: 0005765); |
| | SUBUNIT D 1 | (PC00121) | transporter activity (GO: 0015078) | | proton- |
| | (PTHR11028: SF0) | | | | transporting two- |
| | | | | | sector ATPase |
| | | | | | complex |
| | | | | | (GO: 0016469) |
| AT5G05000 | GTP-BINDING | | | | |
| | PROTEIN A | | | | |
| | (PTHR10903: SF65) | | | | |
| AT5G54750 | TRAFFICKING | | GDP binding (GO: 0019003); GTP binding | ER to Golgi vesicle-mediated | TRAPP complex |
| | PROTEIN | | (GO: 0005525); Rab guanyl-nucleotide | transport (GO: 0006888); | (GO: 0030008); |
| | PARTICLE | | exchange factor activity (GO: 0017112) | intra-Golgi vesicle-mediated | bounding |
| | COMPLEX | | | transport (GO: 0006891) | membrane of |
| | SUBUNIT | | | | organelle |
| | (PTHR13048: SF0) | | | | (GO: 0098588); |
| | | | | | cis-Golgi network |
| | | | | | (GO: 0005801); |
| | | | | | cytosol |
| | | | | | (GO: 0005829); |
| | | | | | plasma membrane |
| | | | | | (GO: 0005886); |
| | | | | | vacuole |
| | | | | | (GO: 0005773) |
| AT1G01100 | 60S ACIDIC | | | | |
| | RIBOSOMAL | | | | |
| | PROTEIN P1-1- | | | | |
| | RELATED | | | | |
| | (PTHR45696: SF16) | | | | |
| AT3G61240 | DEAD-BOX | | | | |
| | ATP- | | | | |
| | DEPENDENT | | | | |
| | RNA HELICASE 12 | | | | |
| | (PTHR24031: SF615) | | | | |
| AT5G64150 | SUBFAMILY | | | | |
| | NOT NAMED | | | | |
| | (PTHR47441: SF3) | | | | |
| AT1G12920 | EUKARYOTIC | translation release | mRNA binding (GO: 0003729) | cytoplasmic translation | cytosol |
| | PEPTIDE CHAIN | factor (PC00225) | | (GO: 00002181); formation of | (GO: 0005829); |
| | RELEASE | | | translation initiation ternary | protein-containing |
| | FACTOR | | | complex (GO: 00001677); | complex |
| | SUBUNIT 1-2 | | | translational elongation | (GO: 0032991) |
| | (PTHR10113: SF31) | | | (GO: 00006414); translational | |
| | | | | termination (GO: 0006415) | |
| AT3G62870 | 60S | ribosomal protein | RNA binding (GO: 0003723) | maturation of LSU-rRNA | cytosolic large |
| | RIBOSOMAL | (PC00202) | | (GO: 0000470) | ribosomal subunit |
| | PROTEIN L7A- | | | | (GO: 0022625) |
| | 1-RELATED | | | | |
| | (PTHR23105: SF122) | | | | |
| AT2G16360 | RIBOSOMAL | | | | |
| | PROTEIN S25 | | | | |
| | (PTHR12850: SF5) | | | | |
| AT2G31060 | ELONGATION | G-protein | GTPase activity (GO: 0003924) | | cytosol |
| | FACTOR | (PC00020); | | | (GO: 0005829); |
| | FAMILY | hydrolase | | | ribonucleoprotein |
| | PROTEIN | (PC00121); | | | complex |
| | (PTHR42908: SF8) | translation | | | (GO: 1990904) |
| | | elongation factor | | | |
| | | (PC00222); | | | |
| | | translation | | | |
| | | initiation factor | | | |
| | | (PC00224) | | | |
| AT1G27900 | PRE-MRNA- | RNA helicase | ATP-dependent helicase activity | mRNA splicing, via | |
| | SPLICING | (PC00032) | (GO: 0008026); RNA binding | spliceosome (GO: 0000398) | |
| | FACTOR ATP- | | (GO: 0003723); catalytic activity, acting | | |
| | DEPENDENT | | on RNA (GO: 0140098) | | |
| | RNA HELICASE | | | | |
| | DEAH4- | | | | |
| | RELATED | | | | |
| | (PTHR18934: SF131) | | | | |
| AT2G43410 | FLOWERING | | | | |
| | TIME CONTROL | | | | |
| | PROTEIN FPA | | | | |
| | (PTHR23189: SF45) | | | | |
| AT3G62310 | DHX15 | RNA helicase | ATP-dependent helicase activity | | |
| | ORTHOLOG, | (PC00032) | (GO: 0008026); RNA binding | | |
| | ISOFORM A | | (GO: 0003723); catalytic activity, acting | | |
| | (PTHR18934: SF109) | | on RNA (GO: 0140098) | | |
| AT4G09730 | ATP- | | | | |
| | DEPENDENT | | | | |
| | RNA HELICASE | | | | |
| | DDX28- | | | | |
| | RELATED | | | | |
| | (PTHR24031: SF421) | | | | |
| AT5G62190 | DEAD (ASP- | | | | |
| | GLU-ALA-ASP) | | | | |
| | BOX HELICASE 21 | | | | |
| | (PTHR24031: SF346) | | | | |
| AT1G60095 | JACALIN- | | | | |
| | RELATED | | | | |
| | LECTIN 16- | | | | |
| | RELATED | | | | |
| | (PTHR47293: SF11) | | | | |
| AT1G74940 | KINASE, | | | | |
| | PUTATIVE | | | | |
| | (DUF581)- | | | | |
| | RELATED | | | | |
| | (PTHR47208: SF7) | | | | |
| AT2G20010 | GLS PROTEIN | | | | |
| | (DUF810) | | | | |
| | (PTHR31280: SF16) | | | | |
| AT2G28620 | KINESIN-LKE | microtubule | ATP-dependent microtubule motor | microtubule-based movement | microtubule |
| | PROTEIN KIN-5A | binding motor | activity, plus-end-directed (GO: 0008574); | (GO: 0007018) | associated |
| | (PTHR24115: SF885) | protein | microtubule binding (GO: 0008017) | | complex |
| | | (PC00156) | | | (GO: 0005875); |
| | | | | | microtubule |
| | | | | | (GO: 0005874) |
| AT2G30500 | PROTEIN | | | | |
| | NETWORKED 4B | | | | |
| | (PTHR32258: SF3) | | | | |
| AT3G14075 | SUBFAMILY | | | | |
| | NOT NAMED | | | | |
| | (PTHR46023: SF5) | | | | |
| AT3G28510 | SUBFAMILY | | | | |
| | NOT NAMED | | | | |
| | (PTHR23070: SF88) | | | | |
| AT3G28540 | BCS1 AAA- | | | | |
| | TYPE ATPASE | | | | |
| | (PTHR23070: SF92) | | | | |
| AT3G47630 | PHOSPHATIDATE | | | | |
| | CYTIDYLYLTRANSFERASE, | | | | |
| | MITOCHONDRIAL | | | | |
| | (PTHR13619: SF0) | | | | |
| AT3G53470 | 2,3-BISPHOSPHOGLYCERATE- | | | | |
| | INDEPENDENT | | | | |
| | PHOSPHOGLYCERATE | | | | |
| | MUTASE | | | | |
| | (PTHR36043: SF1) | | | | |
| AT4G10120 | SUCROSE- | | | | |
| | PHOSPHATE | | | | |
| | SYNTHASE 4- | | | | |
| | RELATED | | | | |
| | (PTHR46039: SF1) | | | | |
| AT5G17760 | SUBFAMILY | | | | |
| | NOT NAMED | | | | |
| | (PTHR23070: SF121) | | | | |
| AT5G36225 | | | | | |
| AT5G51830 | FRUCTOKINASE- | carbohydrate | carbohydrate kinase activity | hexose metabolic process | cytosol |
| | 7-RELATED | kinase | (GO: 0019200); phosphotransferase | (GO: 0019318) | (GO: 0005829) |
| | (PTHR43085: SF7) | (PC00065); | activity, alcohol group as acceptor | | |
| | | nucleotide kinase | (GO: 0016773) | | |
| | | (PC00172) | | | |

### GO-Biological Process

| Enrichment | Genes in | Total | Functional | |
|---|---|---|---|---|
| FDR | list | genes | Category | Genes |
| 2.34E−11 | 35 | 1284 | Response to biotic | AT2G37710 AT4G26120 AT5G45110 AT3G44480 AT3G52430 |
| | | | stimulus | AT4G26070 AT1G02450 AT2G14610 AT1G02930 AT1G05010 |
| | | | | AT2G03440 AT3G01290 AT3G11340 AT3G12580 AT3G26830 |
| | | | | AT3G48090 AT3G50930 AT4G31500 AT4G34135 AT4G37990 |
| | | | | AT5G02490 AT5G10450 AT5G24530 AT2G17265 AT1G51660 |
| | | | | AT2G14560 AT2G26560 AT2G30110 AT3G14840 AT3G48990 |
| | | | | AT4G13350 AT4G16950 AT4G39090 AT5G44070 AT5G54310 |
| 2.34E−11 | 35 | 1271 | Response to external | AT2G37710 AT4G26120 AT5G45110 AT3G44480 AT3G52430 |
| | | | biotic stimulus | AT4G26070 AT1G02450 AT1G02930 AT1G05010 AT2G03440 |
| | | | | AT2G14610 AT3G01290 AT3G11340 AT3G12580 AT3G26830 |
| | | | | AT3G48090 AT3G50930 AT4G31500 AT4G34135 AT4G37990 |
| | | | | AT5G02490 AT5G10450 AT5G24530 AT2G17265 AT1G51660 |
| | | | | AT2G14560 AT2G26560 AT2G30110 AT3G14840 AT3G48990 |
| | | | | AT4G13350 AT4G16950 AT4G39090 AT5G44070 AT5G54310 |
| 2.34E−11 | 35 | 1271 | Response to other | AT2G37710 AT4G26120 AT5G45110 AT3G44480 AT3G52430 |
| | | | organism | AT4G26070 AT1G02450 AT1G02930 AT1G05010 AT2G03440 |
| | | | | AT2G14610 AT3G01290 AT3G11340 AT3G12580 AT3G26830 |
| | | | | AT3G48090 AT3G50930 AT4G31500 AT4G34135 AT4G37990 |
| | | | | AT5G02490 AT5G10450 AT5G24530 AT2G17265 AT1G51660 |
| | | | | AT2G14560 AT2G26560 AT2G30110 AT3G14840 AT3G48990 |
| | | | | AT4G13350 AT4G16950 AT4G39090 AT5G44070 AT5G54310 |
| 2.70E−11 | 40 | 1702 | Response to external | AT2G37710 AT4G26120 AT5G45110 AT3G44480 AT3G52430 |
| | | | stimulus | AT4G26070 AT1G02450 AT4G05420 AT4G21100 AT5G14250 |
| | | | | AT1G02930 AT1G05010 AT1G78380 AT2G03440 AT2G14610 |
| | | | | AT3G01290 AT3G11340 AT3G12580 AT3G26830 AT3G48090 |
| | | | | AT3G50930 AT4G31500 AT4G34135 AT4G37990 AT5G02490 |
| | | | | AT5G10450 AT5G24530 AT5G57560 AT2G17265 AT1G51660 |
| | | | | AT2G14560 AT2G26560 AT2G30110 AT3G14840 AT3G48990 |
| | | | | AT4G13350 AT4G16950 AT4G39090 AT5G44070 AT5G54310 |
| 3.28E−11 | 60 | 3648 | Response to stress | AT1G05010 AT1G16890 AT1G51660 AT1G60140 AT2G29570 |
| | | | | AT2G30110 AT2G37710 AT3G12580 AT3G14890 AT4G15802 |
| | | | | AT4G26070 AT4G26120 AT5G02490 AT5G35530 AT5G44070 |
| | | | | AT5G45110 AT5G50340 AT1G02930 AT1G02450 AT1G63460 |
| | | | | AT2G03440 AT2G14610 AT2G17265 AT2G26560 AT3G07930 |
| | | | | AT3G11340 AT3G26830 AT3G44480 AT3G48090 AT3G48990 |
| | | | | AT3G50930 AT3G52430 AT4G05420 AT4G16950 AT4G19510 |
| | | | | AT4G21100 AT5G24530 AT5G45510 AT5G48620 AT1G04980 |
| | | | | AT1G54100 AT1G55450 AT1G76680 AT1G78380 AT2G24850 |
| | | | | AT3G19420 AT4G31500 AT4G37760 AT4G39090 AT5G03630 |
| | | | | AT5G10450 AT5G18170 AT5G43060 AT5G49570 AT5G57560 |
| | | | | AT2G14560 AT3G06010 AT3G09350 AT3G14840 AT5G54310 |
| 7.11E−10 | 21 | 502 | Response to | AT2G37710 AT4G26120 AT5G45110 AT3G52430 AT4G26070 |
| | | | bacterium | AT1G02930 AT2G03440 AT3G12580 AT3G26830 AT3G50930 |
| | | | | AT4G31500 AT4G37990 AT5G02490 AT5G10450 AT5G24530 |
| | | | | AT2G17265 AT1G02450 AT3G48090 AT4G39090 AT5G44070 |
| | | | | AT5G54310 |
| 3.71E−09 | 38 | 1846 | Multi-organism | AT2G37710 AT4G26120 AT5G45110 AT3G44480 AT3G52430 |
| | | | process | AT4G26070 AT1G02450 AT1G02930 AT1G05010 AT2G03440 |
| | | | | AT2G14610 AT3G01290 AT3G11340 AT3G12580 AT3G26830 |
| | | | | AT3G48090 AT3G50930 AT4G31500 AT4G34135 AT4G37990 |
| | | | | AT5G02490 AT5G10450 AT5G24530 AT2G17265 AT1G51660 |
| | | | | AT2G14560 AT2G26560 AT2G30110 AT2G34480 AT3G14840 |
| | | | | AT3G18520 AT3G48990 AT4G13350 AT4G16950 AT4G39090 |
| | | | | AT5G44070 AT5G54310 AT5G57800 |
| 3.20E−07 | 31 | 1514 | Defense response | AT2G37710 AT4G26120 AT5G45110 AT4G26070 AT1G02450 |
| | | | | AT1G02930 AT1G05010 AT1G51660 AT2G03440 AT2G14610 |
| | | | | AT2G17265 AT2G26560 AT3G11340 AT3G26830 AT3G44480 |
| | | | | AT3G48090 AT3G48990 AT3G50930 AT3G52430 AT4G16950 |
| | | | | AT4G19510 AT5G24530 AT5G45510 AT5G48620 AT4G31500 |
| | | | | AT5G10450 AT2G14560 AT3G14840 AT4G39090 AT5G44070 |
| | | | | AT5G54310 |
| 6.24E−07 | 17 | 485 | Response to metal | AT1G05010 AT5G44070 AT1G02930 AT1G76680 AT1G78380 |
| | | | ion | AT2G21790 AT2G26560 AT2G30110 AT3G12580 AT3G48990 |
| | | | | AT5G02490 AT5G03630 AT5G10450 AT5G18170 AT5G51830 |
| | | | | AT1G16890 AT3G43810 |
| 6.24E−07 | 25 | 1058 | Cellular response to | AT1G05010 AT1G16890 AT1G51660 AT1G60140 AT2G29570 |
| | | | stress | AT2G30110 AT3G12580 AT3G14890 AT4G15802 AT4G26070 |
| | | | | AT5G02490 AT5G35530 AT5G50340 AT3G07930 AT3G50930 |
| | | | | AT3G52430 AT4G05420 AT4G21100 AT5G48620 AT1G04980 |
| | | | | AT1G78380 AT2G26560 AT3G48090 AT4G31500 AT5G44070 |
| 6.24E−07 | 47 | 3184 | Cellular response to | AT1G05010 AT1G16890 AT1G21210 AT1G51660 AT1G60140 |
| | | | stimulus | AT2G16600 AT2G29570 AT2G30110 AT3G12580 AT3G14890 |
| | | | | AT3G43810 AT4G15802 AT4G26070 AT4G26120 AT5G02490 |
| | | | | AT5G35530 AT5G44070 AT5G45110 AT5G50340 AT3G52430 |
| | | | | AT1G01960 AT1G08420 AT1G48210 AT1G63460 AT1G66090 |
| | | | | AT1G72910 AT1G78380 AT2G03440 AT2G37710 AT3G04210 |
| | | | | AT3G07930 AT3G44480 AT3G50930 AT4G02510 AT4G05420 |
| | | | | AT4G16950 AT4G19510 AT4G21100 AT5G14250 AT5G41750 |
| | | | | AT5G48620 AT1G04980 AT2G26560 AT2G37970 AT3G48090 |
| | | | | AT4G31500 AT5G10450 |
| 7.29E−07 | 24 | 995 | Response to | AT1G05010 AT5G44070 AT4G26070 AT1G02930 AT1G54100 |
| | | | inorganic substance | AT1G76680 AT1G78380 AT2G14610 AT2G21790 AT2G26560 |
| | | | | AT2G30110 AT3G12580 AT3G26830 AT3G48990 AT5G02490 |
| | | | | AT5G03630 AT5G10450 AT5G18170 AT5G49570 AT5G51830 |
| | | | | AT3G48090 AT1G16890 AT3G06010 AT3G43810 |
| 1.01E−06 | 15 | 385 | Innate immune | AT1G02450 AT1G51660 AT3G50930 AT3G52430 AT4G26070 |
| | | | response | AT5G48620 AT2G14610 AT2G26560 AT3G26830 AT3G48090 |
| | | | | AT4G31500 AT5G45110 AT3G14840 AT3G44480 AT4G16950 |
| 1.12E−06 | 24 | 1026 | Defense response to | AT2G37710 AT4G26120 AT5G45110 AT4G26070 AT1G02450 |
| | | | other organism | AT1G02930 AT2G14610 AT3G26830 AT3G48090 AT3G52430 |
| | | | | AT4G31500 AT5G10450 AT1G51660 AT2G03440 AT2G14560 |
| | | | | AT2G26560 AT3G14840 AT3G44480 AT3G48990 AT4G16950 |
| | | | | AT4G39090 AT5G24530 AT5G44070 AT5G54310 |
| 1.15E−06 | 15 | 393 | Immune response | AT1G02450 AT1G51660 AT3G50930 AT3G52430 AT4G26070 |
| | | | | AT5G48620 AT2G14610 AT2G26560 AT3G26830 AT3G48090 |
| | | | | AT4G31500 AT5G45110 AT3G14840 AT3G44480 AT4G16950 |
| 1.30E−06 | 11 | 190 | Defense response, | AT1G02450 AT2G14610 AT3G26830 AT3G48090 AT3G52430 |
| | | | incompatible | AT4G26070 AT5G45110 AT1G51660 AT3G14840 AT3G44480 |
| | | | interaction | AT4G16950 |
| 1.58E−06 | 14 | 348 | Response to | AT5G44070 AT1G02930 AT1G76680 AT1G78380 AT2G21790 |
| | | | cadmium ion | AT2G26560 AT2G30110 AT3G12580 AT3G48990 AT5G02490 |
| | | | | AT5G03630 AT5G10450 AT5G18170 AT5G51830 |
| 2.89E−06 | 15 | 430 | Immune system | AT1G02450 AT1G51660 AT3G50930 AT3G52430 AT4G26070 |
| | | | process | AT5G48620 AT2G14610 AT2G26560 AT3G26830 AT3G48090 |
| | | | | AT4G31500 AT5G45110 AT3G14840 AT3G44480 AT4G16950 |
| 2.89E−06 | 45 | 3194 | Response to | AT1G05010 AT2G16600 AT3G12580 AT4G26120 AT5G02490 |
| | | | chemical | AT5G44070 AT5G45110 AT1G16350 AT1G59610 AT3G48990 |
| | | | | AT3G52430 AT4G26070 AT1G02930 AT1G08420 AT1G63460 |
| | | | | AT1G78380 AT2G03440 AT1G54100 AT1G74940 AT1G76680 |
| | | | | AT2G14560 AT2G14610 AT2G21790 AT2G24850 AT2G26560 |
| | | | | AT2G30110 AT2G37710 AT3G26830 AT3G50930 AT4G13180 |
| | | | | AT4G33050 AT4G37760 AT5G03630 AT5G10450 AT5G18170 |
| | | | | AT5G24520 AT5G24530 AT5G49570 AT5G51830 AT5G57560 |
| | | | | AT3G48090 AT1G16890 AT2G02560 AT3G06010 AT3G43810 |
| 2.89E−06 | 15 | 431 | Defense response to | AT2G37710 AT4G26120 AT5G45110 AT4G26070 AT1G02930 |
| | | | bacterium | AT4G31500 AT5G10450 AT1G02450 AT2G03440 AT3G48090 |
| | | | | AT3G52430 AT4G39090 AT5G24530 AT5G44070 AT5G54310 |
| 1.07E−05 | 34 | 2150 | Response to abiotic | AT1G05010 AT2G16600 AT3G07930 AT3G12580 AT4G15802 |
| | | | stimulus | AT5G02490 AT2G03440 AT4G05420 AT4G21100 AT5G14250 |
| | | | | AT1G02930 AT1G54100 AT1G55450 AT1G78380 AT2G14610 |
| | | | | AT2G26560 AT3G19420 AT3G26830 AT3G50930 AT4G39090 |
| | | | | AT5G03630 AT5G18170 AT5G35530 AT5G43060 AT5G57560 |
| | | | | AT5G10450 AT2G37970 AT3G06010 AT3G09350 AT3G43810 |
| | | | | AT3G48090 AT3G52430 AT4G26070 AT4G31500 |
| 5.44E−05 | 15 | 548 | Proteolysis involved | AT2G47110 AT3G07990 AT4G05420 AT4G21100 AT4G39090 |
| | | | in cellular protein | AT5G14250 AT5G43060 AT5G49570 AT5G60360 AT1G16890 |
| | | | catabolic process | AT1G04860 AT3G20630 AT3G58040 AT4G30890 AT5G47040 |
| 6.36E−05 | 15 | 557 | Cellular protein | AT2G47110 AT3G07990 AT4G05420 AT4G21100 AT4G39090 |
| | | | catabolic process | AT5G14250 AT5G43060 AT5G49570 AT5G60360 AT1G16890 |
| | | | | AT1G04860 AT3G20630 AT3G58040 AT4G30890 AT5G47040 |
| 7.62E−05 | 19 | 886 | Organonitrogen | AT2G47110 AT3G07990 AT4G05420 AT4G21100 AT4G39090 |
| | | | compound catabolic | AT5G14250 AT5G43060 AT5G49570 AT5G60360 AT1G16890 |
| | | | process | AT5G48180 AT1G04860 AT1G70580 AT3G20630 AT3G58040 |
| | | | | AT4G30890 AT5G47040 AT5G10450 AT5G44070 |
| 7.92E−05 | 16 | 647 | Protein catabolic | AT2G47110 AT3G07990 AT4G05420 AT4G21100 AT4G39090 |
| | | | process | AT5G14250 AT5G43060 AT5G49570 AT5G60360 AT1G16890 |
| | | | | AT1G04860 AT3G20630 AT3G58040 AT4G30890 AT5G47040 |
| | | | | AT5G10450 |
| 0.00011708 | 12 | 378 | Response to organic | AT2G16600 AT3G52430 AT1G08420 AT1G76680 AT2G14560 |
| | | | cyclic compound | AT2G14610 AT2G37710 AT5G24530 AT5G49570 AT5G57560 |
| | | | | AT3G50930 AT5G10450 |
| 0.00011827 | 28 | 1779 | Response to oxygen- | AT1G05010 AT2G16600 AT4G26120 AT5G45110 AT1G59610 |
| | | | containing | AT3G52430 AT4G26070 AT1G08420 AT2G03440 AT1G02930 |
| | | | compound | AT1G54100 AT1G76680 AT1G78380 AT2G14560 AT2G14610 |
| | | | | AT2G24850 AT2G37710 AT3G12580 AT3G26830 AT4G33050 |
| | | | | AT4G37760 AT5G24530 AT5G49570 AT5G57560 AT3G48090 |
| | | | | AT3G06010 AT3G50930 AT5G10450 |
| 0.00022335 | 16 | 709 | Cellular | AT1G68200 AT2G47110 AT3G07990 AT4G05420 AT4G21100 |
| | | | macromolecule | AT4G39090 AT5G14250 AT5G43060 AT5G49570 AT5G60360 |
| | | | catabolic process | AT1G16890 AT1G04860 AT3G20630 AT3G58040 AT4G30890 |
| | | | | AT5G47040 |
| 0.00022852 | 6 | 79 | Systemic acquired | AT1G02450 AT2G14610 AT3G26830 AT3G48090 AT3G52430 |
| | | | resistance | AT5G45110 |
| 0.00028608 | 3 | 8 | Leaf abscission | AT3G48090 AT3G52430 AT5G54310 |

### GO-Molecular Function

| Enrichment | Genes in | Total | Functional | |
|---|---|---|---|---|
| FDR | list | genes | Category | Genes |
| 5.98E−12 | 59 | 3379 | Small molecule | AT2G04430 AT3G09350 AT3G12580 AT4G02510 AT5G02490 |
| | | | binding | AT5G05000 AT5G22140 AT1G76680 AT4G13350 AT5G18170 |
| | | | | AT5G45110 AT1G05010 AT1G16350 AT1G16890 AT1G21210 |
| | | | | AT1G27900 AT1G48210 AT1G51660 AT1G59610 AT1G66090 |
| | | | | AT1G70580 AT1G72910 AT2G01490 AT2G17265 AT2G21470 |
| | | | | AT2G21790 AT2G24850 AT2G28620 AT2G29720 AT2G30110 |
| | | | | AT2G31060 AT2G37710 AT3G04210 AT3G06010 AT3G13870 |
| | | | | AT3G14840 AT3G19960 AT3G28510 AT3G28540 AT3G44480 |
| | | | | AT3G48990 AT3G50930 AT3G61240 AT3G62310 AT4G09730 |
| | | | | AT4G16950 AT4G19510 AT4G26070 AT4G37760 AT5G03630 |
| | | | | AT5G17760 AT5G40760 AT5G41750 AT5G45510 AT5G47040 |
| | | | | AT5G48620 AT5G50340 AT5G51830 AT5G62190 |
| 1.02E−11 | 58 | 3370 | Anion binding | AT1G02930 AT3G12580 AT4G02510 AT5G02490 AT5G05000 |
| | | | | AT5G22140 AT1G76680 AT1G78380 AT3G19420 AT4G13350 |
| | | | | AT5G18170 AT5G45110 AT1G05010 AT1G16890 AT1G21210 |
| | | | | AT1G27900 AT1G48210 AT1G51660 AT1G59610 AT1G66090 |
| | | | | AT1G70580 AT1G72910 AT2G01490 AT2G17265 AT2G21470 |
| | | | | AT2G21790 AT2G24850 AT2G28620 AT2G29720 AT2G30110 |
| | | | | AT2G31060 AT2G37710 AT3G04210 AT3G06010 AT3G13870 |
| | | | | AT3G14840 AT3G19960 AT3G28510 AT3G28540 AT3G44480 |
| | | | | AT3G48990 AT3G50930 AT3G61240 AT3G62310 AT4G09730 |
| | | | | AT4G16950 AT4G19510 AT4G26070 AT4G37760 AT5G03630 |
| | | | | AT5G17760 AT5G41750 AT5G45510 AT5G47040 AT5G48620 |
| | | | | AT5G50340 AT5G51830 AT5G62190 |
| 7.71E−11 | 54 | 3169 | Nucleotide binding | AT2G04430 AT3G09350 AT3G12580 AT4G02510 AT5G02490 |
| | | | | AT5G05000 AT5G22140 AT1G76680 AT4G13350 AT5G18170 |
| | | | | AT1G16350 AT1G16890 AT1G21210 AT1G27900 AT1G48210 |
| | | | | AT1G51660 AT1G59610 AT1G66090 AT1G72910 AT2G17265 |
| | | | | AT2G21470 AT2G21790 AT2G28620 AT2G29720 AT2G30110 |
| | | | | AT2G31060 AT2G37710 AT3G04210 AT3G06010 AT3G13870 |
| | | | | AT3G14840 AT3G19960 AT3G28510 AT3G28540 AT3G44480 |
| | | | | AT3G48990 AT3G50930 AT3G61240 AT3G62310 AT4G09730 |
| | | | | AT4G16950 AT4G19510 AT4G26070 AT4G37760 AT5G03630 |
| | | | | AT5G17760 AT5G40760 AT5G41750 AT5G45510 AT5G47040 |
| | | | | AT5G48620 AT5G50340 AT5G51830 AT5G62190 |
| 7.71E−11 | 54 | 3169 | Nucleoside | AT2G04430 AT3G09350 AT3G12580 AT4G02510 AT5G02490 |
| | | | phosphate binding | AT5G05000 AT5G22140 AT1G76680 AT4G13350 AT5G18170 |
| | | | | AT1G16350 AT1G16890 AT1G21210 AT1G27900 AT1G48210 |
| | | | | AT1G51660 AT1G59610 AT1G66090 AT1G72910 AT2G17265 |
| | | | | AT2G21470 AT2G21790 AT2G28620 AT2G29720 AT2G30110 |
| | | | | AT2G31060 AT2G37710 AT3G04210 AT3G06010 AT3G13870 |
| | | | | AT3G14840 AT3G19960 AT3G28510 AT3G28540 AT3G44480 |
| | | | | AT3G48990 AT3G50930 AT3G61240 AT3G62310 AT4G09730 |
| | | | | AT4G16950 AT4G19510 AT4G26070 AT4G37760 AT5G03630 |
| | | | | AT5G17760 AT5G40760 AT5G41750 AT5G45510 AT5G47040 |
| | | | | AT5G48620 AT5G50340 AT5G51830 AT5G62190 |
| 1.44E−08 | 47 | 2894 | Carbohydrate | AT3G12580 AT4G02510 AT5G02490 AT5G05000 AT1G02930 |
| | | | derivative binding | AT1G76680 AT4G13350 AT5G18170 AT1G16890 AT1G21210 |
| | | | | AT1G27900 AT1G48210 AT1G51660 AT1G59610 AT1G66090 |
| | | | | AT1G72910 AT2G17265 AT2G21470 AT2G21790 AT2G28620 |
| | | | | AT2G30110 AT2G31060 AT2G37710 AT3G04210 AT3G06010 |
| | | | | AT3G13870 AT3G14840 AT3G19960 AT3G28510 AT3G28540 |
| | | | | AT3G44480 AT3G48990 AT3G50930 AT3G61240 AT3G62310 |
| | | | | AT4G09730 AT4G16950 AT4G19510 AT4G26070 AT5G17760 |
| | | | | AT5G41750 AT5G45510 AT5G47040 AT5G48620 AT5G50340 |
| | | | | AT5G51830 AT5G62190 |
| 1.92E−08 | 46 | 2830 | Purine nucleotide | AT3G09350 AT3G12580 AT4G02510 AT5G02490 AT5G05000 |
| | | | binding | AT4G13350 AT5G18170 AT1G16890 AT1G21210 AT1G27900 |
| | | | | AT1G48210 AT1G51660 AT1G59610 AT1G66090 AT1G72910 |
| | | | | AT2G17265 AT2G21470 AT2G21790 AT2G28620 AT2G30110 |
| | | | | AT2G31060 AT2G37710 AT3G04210 AT3G06010 AT3G13870 |
| | | | | AT3G14840 AT3G19960 AT3G28510 AT3G28540 AT3G44480 |
| | | | | AT3G48990 AT3G50930 AT3G61240 AT3G62310 AT4G09730 |
| | | | | AT4G16950 AT4G19510 AT4G26070 AT5G17760 AT5G41750 |
| | | | | AT5G45510 AT5G47040 AT5G48620 AT5G50340 AT5G51830 |
| | | | | AT5G62190 |
| 2.12E−08 | 46 | 2852 | Ribonucleotide | AT3G12580 AT4G02510 AT5G02490 AT5G05000 AT1G76680 |
| | | | binding | AT4G13350 AT5G18170 AT1G16890 AT1G21210 AT1G27900 |
| | | | | AT1G48210 AT1G51660 AT1G59610 AT1G66090 AT1G72910 |
| | | | | AT2G17265 AT2G21470 AT2G21790 AT2G28620 AT2G30110 |
| | | | | AT2G31060 AT2G37710 AT3G04210 AT3G06010 AT3G13870 |
| | | | | AT3G14840 AT3G19960 AT3G28510 AT3G28540 AT3G44480 |
| | | | | AT3G48990 AT3G50930 AT3G61240 AT3G62310 AT4G09730 |
| | | | | AT4G16950 AT4G19510 AT4G26070 AT5G17760 AT5G41750 |
| | | | | AT5G45510 AT5G47040 AT5G48620 AT5G50340 AT5G51830 |
| | | | | AT5G62190 |
| 4.36E−08 | 45 | 2823 | Purine | AT3G12580 AT4G02510 AT5G02490 AT5G05000 AT4G13350 |
| | | | ribonucleotide | AT5G18170 AT1G16890 AT1G21210 AT1G27900 AT1G48210 |
| | | | binding | AT1G51660 AT1G59610 AT1G66090 AT1G72910 AT2G17265 |
| | | | | AT2G21470 AT2G21790 AT2G28620 AT2G30110 AT2G31060 |
| | | | | AT2G37710 AT3G04210 AT3G06010 AT3G13870 AT3G14840 |
| | | | | AT3G19960 AT3G28510 AT3G28540 AT3G44480 AT3G48990 |
| | | | | AT3G50930 AT3G61240 AT3G62310 AT4G09730 AT4G16950 |
| | | | | AT4G19510 AT4G26070 AT5G17760 AT5G41750 AT5G45510 |
| | | | | AT5G47040 AT5G48620 AT5G50340 AT5G51830 AT5G62190 |
| 5.02E−08 | 25 | 987 | Pyrophosphatase | AT1G27900 AT1G59610 AT2G04430 AT2G28620 AT2G31060 |
| | | | activity | AT3G12580 AT3G13870 AT3G28710 AT3G62310 AT5G02490 |
| | | | | AT4G09730 AT4G13350 AT5G44070 AT3G06010 AT3G19960 |
| | | | | AT3G28510 AT3G28540 AT3G50930 AT3G61240 AT5G17760 |
| | | | | AT5G47040 AT5G50340 AT5G62190 AT4G02510 AT5G05000 |
| 5.42E−08 | 25 | 996 | Hydrolase activity, | AT1G27900 AT1G59610 AT2G04430 AT2G28620 AT2G31060 |
| | | | acting on acid | AT3G12580 AT3G13870 AT3G28710 AT3G62310 AT5G02490 |
| | | | anhydrides, in | AT4G09730 AT4G13350 AT5G44070 AT3G06010 AT3G19960 |
| | | | phosphorus- | AT3G28510 AT3G28540 AT3G50930 AT3G61240 AT5G17760 |
| | | | containing | AT5G47040 AT5G50340 AT5G62190 AT4G02510 AT5G05000 |
| | | | anhydrides | |
| 5.67E−08 | 25 | 1003 | Hydrolase activity, | AT1G27900 AT1G59610 AT2G04430 AT2G28620 AT2G31060 |
| | | | acting on acid | AT3G12580 AT3G13870 AT3G28710 AT3G62310 AT5G02490 |
| | | | anhydrides | AT4G09730 AT4G13350 AT5G44070 AT3G06010 AT3G19960 |
| | | | | AT3G28510 AT3G28540 AT3G50930 AT3G61240 AT4G02510 |
| | | | | AT5G17760 AT5G47040 AT5G50340 AT5G62190 AT5G05000 |
| 5.91E−08 | 24 | 932 | Nucleoside- | AT1G27900 AT1G59610 AT2G28620 AT2G31060 AT3G12580 |
| | | | triphosphatase | AT3G13870 AT3G28710 AT3G62310 AT5G02490 AT4G09730 |
| | | | activity | AT4G13350 AT5G44070 AT3G06010 AT3G19960 AT3G28510 |
| | | | | AT3G28540 AT3G50930 AT3G61240 AT5G17760 AT5G47040 |
| | | | | AT5G50340 AT5G62190 AT4G02510 AT5G05000 |
| 4.60E−07 | 40 | 2556 | Adenyl nucleotide | AT3G09350 AT3G12580 AT5G02490 AT5G18170 AT1G16890 |
| | | | binding | AT1G21210 AT1G27900 AT1G48210 AT1G51660 AT1G66090 |
| | | | | AT1G72910 AT2G17265 AT2G21470 AT2G21790 AT2G28620 |
| | | | | AT2G30110 AT2G37710 AT3G04210 AT3G06010 AT3G14840 |
| | | | | AT3G19960 AT3G28510 AT3G28540 AT3G44480 AT3G48990 |
| | | | | AT3G50930 AT3G61240 AT3G62310 AT4G09730 AT4G16950 |
| | | | | AT4G19510 AT4G26070 AT5G17760 AT5G41750 AT5G45510 |
| | | | | AT5G47040 AT5G48620 AT5G50340 AT5G51830 AT5G62190 |
| 6.17E−07 | 40 | 2592 | Drug binding | AT2G16600 AT3G12580 AT5G02490 AT1G02930 AT5G18170 |
| | | | | AT5G45110 AT1G16890 AT1G21210 AT1G27900 AT1G48210 |
| | | | | AT1G51660 AT1G70580 AT2G17265 AT2G21470 AT2G21790 |
| | | | | AT2G24850 AT2G28620 AT2G30110 AT2G37710 AT3G06010 |
| | | | | AT3G14840 AT3G19960 AT3G28510 AT3G28540 AT3G44480 |
| | | | | AT3G48990 AT3G50930 AT3G61240 AT3G62310 AT4G09730 |
| | | | | AT4G16950 AT4G19510 AT4G26070 AT5G17760 AT5G45510 |
| | | | | AT5G47040 AT5G48620 AT5G50340 AT5G51830 AT5G62190 |
| 6.17E−07 | 41 | 2704 | Purine | AT3G12580 AT4G02510 AT5G02490 AT5G05000 AT4G13350 |
| | | | ribonucleoside | AT5G18170 AT1G16890 AT1G21210 AT1G27900 AT1G48210 |
| | | | triphosphate | AT1G51660 AT1G59610 AT2G17265 AT2G21470 AT2G21790 |
| | | | binding | AT2G28620 AT2G30110 AT2G31060 AT2G37710 AT3G06010 |
| | | | | AT3G13870 AT3G14840 AT3G19960 AT3G28510 AT3G28540 |
| | | | | AT3G44480 AT3G48990 AT3G50930 AT3G61240 AT3G62310 |
| | | | | AT4G09730 AT4G16950 AT4G19510 AT4G26070 AT5G17760 |
| | | | | AT5G45510 AT5G47040 AT5G48620 AT5G50340 AT5G51830 |
| | | | | AT5G62190 |
| 1.07E−06 | 39 | 2550 | Adenyl | AT3G12580 AT5G02490 AT5G18170 AT1G16890 AT1G21210 |
| | | | ribonucleotide | AT1G27900 AT1G48210 AT1G51660 AT1G66090 AT1G72910 |
| | | | binding | AT2G17265 AT2G21470 AT2G21790 AT2G28620 AT2G30110 |
| | | | | AT2G37710 AT3G04210 AT3G06010 AT3G14840 AT3G19960 |
| | | | | AT3G28510 AT3G28540 AT3G44480 AT3G48990 AT3G50930 |
| | | | | AT3G61240 AT3G62310 AT4G09730 AT4G16950 AT4G19510 |
| | | | | AT4G26070 AT5G17760 AT5G41750 AT5G45510 AT5G47040 |
| | | | | AT5G48620 AT5G50340 AT5G51830 AT5G62190 |
| 3.02E−06 | 48 | 3682 | Hydrolase activity | AT1G04860 AT1G27900 AT1G48320 AT1G59610 AT1G60140 |
| | | | | AT2G04430 AT2G26560 AT2G28620 AT2G31060 AT3G07990 |
| | | | | AT3G12580 AT3G13870 AT3G14890 AT3G20630 AT3G28710 |
| | | | | AT3G62310 AT4G30890 AT4G39090 AT5G02490 AT5G35530 |
| | | | | AT5G43060 AT5G49570 AT5G60360 AT3G19420 AT4G09730 |
| | | | | AT4G13350 AT4G17830 AT5G44070 AT1G08420 AT3G06010 |
| | | | | AT3G07930 AT3G14075 AT3G18520 AT3G19960 AT3G23570 |
| | | | | AT3G28510 AT3G28540 AT3G48090 AT3G50930 AT3G52430 |
| | | | | AT3G61240 AT4G02510 AT5G05000 AT5G17760 AT5G47040 |
| | | | | AT5G50340 AT5G57560 AT5G62190 |
| 2.10E−05 | 35 | 2433 | ATP binding | AT3G12580 AT5G02490 AT5G18170 AT1G16890 AT1G21210 |
| | | | | AT1G27900 AT1G48210 AT1G51660 AT2G17265 AT2G21470 |
| | | | | AT2G21790 AT2G28620 AT2G30110 AT2G37710 AT3G06010 |
| | | | | AT3G14840 AT3G19960 AT3G28510 AT3G28540 AT3G44480 |
| | | | | AT3G48990 AT3G50930 AT3G61240 AT3G62310 AT4G09730 |
| | | | | AT4G16950 AT4G19510 AT4G26070 AT5G17760 AT5G45510 |
| | | | | AT5G47040 AT5G48620 AT5G50340 AT5G51830 AT5G62190 |
| 2.69E−05 | 9 | 187 | ADP binding | AT1G66090 AT1G72910 AT3G04210 AT3G44480 AT4G16950 |
| | | | | AT4G19510 AT5G41750 AT5G45510 AT5G48620 |
| 4.67E−05 | 6 | 70 | Cysteine-type | AT1G04860 AT3G20630 AT4G30890 AT4G39090 AT5G43060 |
| | | | endopeptidase | AT5G60360 |
| | | | activity | |
| 0.000128775 | 14 | 572 | ATPase activity | AT1G27900 AT2G28620 AT3G12580 AT3G28710 AT3G62310 |
| | | | | AT5G02490 AT4G09730 AT5G44070 AT3G28510 AT3G28540 |
| | | | | AT3G50930 AT5G17760 AT5G47040 AT5G50340 |
| 0.000993086 | 2 | 3 | Salicylic acid | AT2G43820 AT3G11340 |
| | | | glucosyltransferase | |
| | | | (glucoside- | |
| | | | forming) activity | |
| 0.002003836 | 11 | 487 | Coenzyme binding | AT2G04430 AT5G22140 AT1G76680 AT1G05010 AT1G70580 |
| | | | | AT2G01490 AT2G24850 AT2G29720 AT4G37760 AT5G03630 |
| | | | | AT5G40760 |
| 0.00204287 | 18 | 1137 | Cofactor binding | AT1G02930 AT2G04430 AT2G37970 AT4G22220 AT5G22140 |
| | | | | AT1G76680 AT1G78380 AT1G05010 AT1G11680 AT1G70580 |
| | | | | AT2G01490 AT2G24850 AT2G29720 AT3G26830 AT4G31500 |
| | | | | AT4G37760 AT5G03630 AT5G40760 |
| 0.003109677 | 4 | 56 | Damaged DNA | AT4G05420 AT4G21100 AT5G35530 AT5G50340 |
| | | | binding | |
| 0.003322351 | 6 | 157 | Cysteine-type | AT1G04860 AT3G20630 AT4G30890 AT4G39090 AT5G43060 |
| | | | peptidase activity | AT5G60360 |
| 0.004086777 | 8 | 299 | Cytoskeletal | AT1G59610 AT2G28620 AT2G30500 AT3G18060 AT2G20190 |
| | | | protein binding | AT2G31200 AT2G41740 AT3G19960 |
| 0.004097246 | 4 | 62 | Actin filament | AT2G30500 AT3G18060 AT2G41740 AT3G19960 |
| | | | binding | |
| 0.00421146 | 22 | 1664 | Oxidoreductase | AT1G05010 AT1G11680 AT1G16350 AT1G54100 AT1G63460 |
| | | | activity | AT1G76680 AT2G21790 AT2G26400 AT3G26830 AT4G31500 |
| | | | | AT4G37760 AT4G37990 AT5G22140 AT5G24530 AT5G57800 |
| | | | | AT5G18170 AT5G40760 AT1G78380 AT2G01490 AT2G29320 |
| | | | | AT2G29720 AT5G03630 |
| 0.005459598 | 5 | 118 | Actin binding | AT2G30500 AT3G18060 AT2G31200 AT2G41740 AT3G19960 |

### GO-Cellular Component

| Enrichment | Genes in | Total | Functional | |
|---|---|---|---|---|
| FDR | list | genes | Category | Genes |
| 6.25E−16 | 54 | 2350 | Cytosol | AT1G01100 AT1G01960 AT1G12920 AT1G60140 AT1G63460 |
| | | | | AT1G68200 AT2G16600 AT2G21790 AT2G31060 AT2G34480 |
| | | | | AT3G06720 AT3G62870 AT4G13350 AT4G15802 AT5G35530 |
| | | | | AT5G48180 AT5G49570 AT5G51830 AT5G54750 AT1G02930 |
| | | | | AT1G16350 AT1G16890 AT1G27970 AT1G54100 AT1G59610 |
| | | | | AT1G74940 AT1G76680 AT1G78380 AT2G02560 AT2G21470 |
| | | | | AT2G30110 AT2G43820 AT2G47110 AT3G12580 AT3G15610 |
| | | | | AT3G18060 AT3G19420 AT3G20630 AT3G23570 AT3G62310 |
| | | | | AT4G02510 AT4G05420 AT4G17830 AT5G02490 AT5G03630 |
| | | | | AT5G10450 AT5G14250 AT5G40760 AT5G43060 AT5G44070 |
| | | | | AT5G45510 AT5G54310 AT4G22220 AT2G04430 |
| 0.00025932 | 40 | 3155 | Plasma membrane | AT1G11680 AT1G21210 AT1G48210 AT2G16600 AT2G37710 |
| | | | | AT3G14840 AT3G51250 AT1G01100 AT1G08050 AT1G08420 |
| | | | | AT1G12920 AT1G16890 AT1G27970 AT1G59610 AT1G78380 |
| | | | | AT2G01490 AT2G02560 AT2G20190 AT2G30110 AT2G34480 |
| | | | | AT2G37970 AT3G01290 AT3G12580 AT3G13870 AT3G19960 |
| | | | | AT3G28710 AT3G44480 AT3G50590 AT4G10120 AT4G31500 |
| | | | | AT5G02490 AT5G10450 AT5G17760 AT5G22140 AT5G24710 |
| | | | | AT5G45500 AT5G45510 AT5G48620 AT5G57800 AT5G61790 |
| 0.00025932 | 46 | 3832 | Cell periphery | AT1G11680 AT1G21210 AT1G48210 AT2G16600 AT2G37710 |
| | | | | AT3G14840 AT3G18060 AT3G51250 AT1G01100 AT1G02930 |
| | | | | AT1G04980 AT1G08050 AT1G08420 AT1G12920 AT1G16890 |
| | | | | AT1G27970 AT1G59610 AT1G78380 AT2G01490 AT2G02560 |
| | | | | AT2G14610 AT2G20190 AT2G30110 AT2G34480 AT2G37970 |
| | | | | AT3G01290 AT3G06720 AT3G12580 AT3G13870 AT3G19960 |
| | | | | AT3G28710 AT3G44480 AT3G50590 AT4G10120 AT4G31500 |
| | | | | AT5G02490 AT5G10450 AT5G17760 AT5G22140 AT5G24710 |
| | | | | AT5G45500 AT5G45510 AT5G48620 AT5G57560 AT5G57800 |
| | | | | AT5G61790 |
| 0.001804845 | 30 | 2283 | Endomembrane | AT1G01960 AT1G07140 AT1G11680 AT1G27970 AT2G16600 |
| | | | system | AT3G06720 AT3G09350 AT3G13870 AT4G37760 AT5G54750 |
| | | | | AT5G57800 AT5G60510 AT1G04980 AT2G37970 AT2G47110 |
| | | | | AT3G12580 AT3G19960 AT3G26830 AT3G28510 AT3G28710 |
| | | | | AT3G44480 AT4G15802 AT4G31500 AT5G02490 AT5G22140 |
| | | | | AT5G43060 AT5G54310 AT5G57560 AT5G61790 AT3G48090 |
| 0.00713953 | 4 | 53 | Lysosome | AT3G28710 AT4G39090 AT5G43060 AT5G60360 |
| 0.007355789 | 4 | 56 | Lytic vacuole | AT3G28710 AT4G39090 AT5G43060 AT5G60360 |
| 0.013389304 | 17 | 1155 | Vacuole | AT2G16600 AT2G30500 AT3G28710 AT3G50590 AT4G39090 |
| | | | | AT5G43060 AT5G60360 AT1G02930 AT1G59610 AT1G78380 |
| | | | | AT2G34480 AT2G37970 AT3G01290 AT3G12580 AT3G13870 |
| | | | | AT3G62870 AT5G61790 |
| 0.017117105 | 16 | 1090 | Endoplasmic | AT1G11680 AT3G09350 AT3G13870 AT4G37760 AT5G57800 |
| | | | reticulum | AT5G60510 AT1G04980 AT3G19960 AT3G26830 AT3G28510 |
| | | | | AT3G44480 AT4G31500 AT5G43060 AT5G61790 AT3G48090 |
| | | | | AT5G54750 |
| 0.0184581 | 15 | 1036 | Cell-cell junction | AT1G02930 AT1G59610 AT2G16600 AT2G20190 AT2G21470 |
| | | | | AT2G30110 AT3G01290 AT3G14840 AT3G28710 AT3G48990 |
| | | | | AT3G51250 AT5G22140 AT5G35530 AT5G61790 AT3G19960 |
| 0.0184581 | 15 | 1036 | Plasmodesma | AT1G02930 AT1G59610 AT2G16600 AT2G20190 AT2G21470 |
| | | | | AT2G30110 AT3G01290 AT3G14840 AT3G28710 AT3G48990 |
| | | | | AT3G51250 AT5G22140 AT5G35530 AT5G61790 AT3G19960 |
| 0.0184581 | 15 | 1036 | Cell junction | AT1G02930 AT1G59610 AT2G16600 AT2G20190 AT2G21470 |
| | | | | AT2G30110 AT3G01290 AT3G14840 AT3G28710 AT3G48990 |
| | | | | AT3G51250 AT5G22140 AT5G35530 AT5G61790 AT3G19960 |
| 0.0184581 | 15 | 1036 | Symplast | AT1G02930 AT1G59610 AT2G16600 AT2G20190 AT2G21470 |
| | | | | AT2G30110 AT3G01290 AT3G14840 AT3G28710 AT3G48990 |
| | | | | AT3G51250 AT5G22140 AT5G35530 AT5G61790 AT3G19960 |
| 0.026223194 | 11 | 674 | Vacuolar | AT2G16600 AT2G30500 AT3G28710 AT3G50590 AT1G78380 |
| | | | membrane | AT2G34480 AT3G01290 AT3G12580 AT3G13870 AT3G62870 |
| | | | | AT5G61790 |
| 0.026223194 | 11 | 676 | Vacuolar part | AT2G16600 AT2G30500 AT3G28710 AT3G50590 AT1G78380 |
| | | | | AT2G34480 AT3G01290 AT3G12580 AT3G13870 AT3G62870 |
| | | | | AT5G61790 |
| 0.030339255 | 15 | 1155 | Golgi apparatus | AT1G01960 AT2G16600 AT5G54750 AT1G11680 AT2G37970 |
| | | | | AT2G47110 AT3G12580 AT3G28710 AT3G44480 AT4G15802 |
| | | | | AT5G02490 AT5G22140 AT5G54310 AT5G57560 AT3G13870 |
| 0.030339255 | 8 | 424 | Cytoskeleton | AT1G07140 AT2G28620 AT3G18060 AT2G20190 AT1G59610 |
| | | | | AT2G31200 AT2G41740 AT3G19960 |
| 0.030339255 | 3 | 56 | Actin cytoskeleton | AT3G18060 AT2G31200 AT3G19960 |
| 0.030339255 | 21 | 1852 | Non-membrane- | AT1G01100 AT1G07140 AT2G28620 AT2G34480 AT3G18060 |
| | | | bounded organelle | AT3G18580 AT3G62870 AT5G35530 AT2G20190 AT2G43410 |
| | | | | AT2G47110 AT3G06720 AT3G62310 AT5G62190 AT1G59610 |
| | | | | AT2G16360 AT2G31200 AT2G41740 AT3G19960 AT3G61240 |
| | | | | AT4G31210 |
| 0.030339255 | 21 | 1852 | Intracellular non- | AT1G01100 AT1G07140 AT2G28620 AT2G34480 AT3G18060 |
| | | | membrane- | AT3G18580 AT3G62870 AT5G35530 AT2G20190 AT2G43410 |
| | | | bounded organelle | AT2G47110 AT3G06720 AT3G62310 AT5G62190 AT1G59610 |
| | | | | AT2G16360 AT2G31200 AT2G41740 AT3G19960 AT3G61240 |
| | | | | AT4G31210 |
| 0.030339255 | 14 | 1029 | Whole membrane | AT2G16600 AT2G30500 AT3G28710 AT3G50590 AT4G02510 |
| | | | | AT1G78380 AT2G34480 AT3G01290 AT3G12580 AT3G13870 |
| | | | | AT3G50930 AT3G62870 AT5G05000 AT5G61790 |
| 0.032406858 | 17 | 1400 | Bounding | AT2G16600 AT2G30500 AT3G28710 AT3G50590 AT4G02510 |
| | | | membrane of | AT5G54750 AT1G01960 AT1G78380 AT2G34480 AT3G01290 |
| | | | organelle | AT3G12580 AT3G13870 AT3G44480 AT3G50930 AT3G62870 |
| | | | | AT5G05000 AT5G61790 |
| 0.038070656 | 3 | 65 | Nuclear pore | AT1G07140 AT1G27970 AT3G06720 |

TABLE 6
List of Primers Used in this Study.

| | | | |
|---|---|---|---|
| | | SEQ | |
| Name | Sequence (5′-3′) | ID NO | Use |

AtNPR1 primers

| | | | |
|---|---|---|---|
| NPR1-C-FB1 | GGGGACAAGTTTGTACAAAAAAGCAGGCTTA | 35 | Truncation variants |
| | ATGGACACCACCATTGATGGATTC | | |
| ANK-F630-B1 | GGGGACAAGTTTGTACAAAAAAGCAGGCTTA | 36 | Truncation variants |
| | ATGATATGTGGTAAAGCTTGTATGAAG | | |
| NPR1-R630-B2 | GGGGACCACTTTGTACAAGAAAGCTGGGTCAT | 37 | Truncation variants |
| | TAGCAAGCTTGAGTATAACCAATG | | |
| N1-ANK-F688-B1 | GGGGACAAGTTTGTACAAAAAAGCAGGCTTA | 38 | Truncation variants |
| | ATGTCTAATGTAGATATGGTTAGTCT | | |
| N1-ANK-R1080B2 | GGGGACCACTTTGTACAAGAAAGCTGGGTCCA | 39 | Truncation variants |
| | AAGTTGCTTCTGATGCACTTG | | |
| N1-F1021-B1 | GGGGACAAGTTTGTACAAAAAAGCAGGCTTA | 40 | Truncation variants |
| | GAGCCACAATTGATACTATCTCTATTGG | | |
| NPR1-R-B2 | GGGGACCACTTTGTACAAGAAAGCTGGGTCCC | 41 | Truncation variants |
| | GACGACGATGAGAGAG | | |
| Nde-GST-F4 | CATACATATGTCCCCTATACTAGGTTATTGGA | 42 | GST/GST-NPR1/sim3 |
| | AAATTAAG | | for DUET vector |
| Avr-GST-R657 | CATACCTAGGTCATTTTGGAGGATGGTCGCCA | 43 | GSTfor DUET vector |
| | CCAC | | |
| Avr-N1-R630 | CATACCTAGGTCAATTAGCAAGCTTGAGTATA | 44 | GST-NPR1/sim3 for |
| | ACCAATG | | DUET vecotr |
| N1-R432K-F | GACGCTGCTCGATCTTGAAAATAAAGTTGCAC | 45 | nim1-4 mutagenesis |
| | TTGC | | |
| N1-R432K-R | GCAAGTGCAACTTTATTTTCAAGATCGAGCAG | 46 | nim1-4 mutagenesis |
| | CGTC | | |

rdr1 (C150/155/156/160A)

| | | | |
|---|---|---|---|
| C150A-F | GCCGCCTAAAGGAGTTTCTGAAGCTGCAGACG | 47 | RDR1 site-directed |
| | AGAAT | | mutagenesis |
| C150A-R | ATTCTCGTCTGCAGCTTCAGAAACTCCTTTAG | 48 | RDR1 site-directed |
| | GCGGC | | mutagenesis |
| C155/156/160A-F | GTTTCTGAAGCTGCAGACGAGAATGCGGCTCA | 49 | RDR1 site-directed |
| | CGTGGCTGCTCGGCCGGCGGTGGATTT | | mutagenesis |
| C155/156/160A-R | AAATCCACCGCCGGCCGAGCAGCCACGTGAG | 50 | RDR1 site-directed |
| | CCGCATTCTCGTCTGCAGCTTCAGAAAC | | mutagenesis |

rdr2 (C378/385/394A)

| | | | |
|---|---|---|---|
| C378A-F | GCCACTATGGCGGTTGAAGCGAATAATATCCC | 51 | RDR2 site-directed |
| | GGAGCAA | | mutagenesis |
| C378A-R | TTGCTCCGGGATATTATTCGCTTCAACCGCCAT | 52 | RDR2 site-directed |
| | AGTGGC | | mutagenesis |
| C385A-F | AATAATATCCCGGAGCAAGCTAAGCATTCTCT | 53 | RDR2 site-directed |
| | CAAAGGCCG | | mutagenesis |
| C385A-R | CGGCCTTTGAGAGAATGCTTAGCTTGCTCCGG | 54 | RDR2 site-directed |
| | GATATTATT | | mutagenesis |
| C394A-F | AAGCATTCTCTCAAAGGCCGACTAGCAGTAGA | 55 | RDR2 site-directed |
| | AATACTAGAGCAAGAAG | | mutagenesis |
| C394A-R | CTTCTTGCTCTAGTATTTCTACTGCTAGTCGGC | 56 | RDR2 site-directed |
| | CTTTGAGAGAATGCTT | | mutagenesis |

rdr3 (C511/521/529A)

| | | | |
|---|---|---|---|
| C511A-F | CGGGAAACGATTCTTCCCGCGCGCATCGGCAG | 57 | RDR3 site-directed |
| | TGCTCGACCAGATTAT | | mutagenesis |
| C511A-R | ATAATCTGGTCGAGCACTGCCGATGCGCGCGG | 58 | RDR3 site-directed |
| | GAAGAATCGTTTCCCG | | mutagenesis |
| C521A-F | TGCTCGACCAGATTATGAACGCAGAGGACTTG | 59 | RDR3 site-directed |
| | ACTCAACTGGCT | | mutagenesis |
| C521A-R | AGCCAGTTGAGTCAAGTCCTCTGCGTTCATAA | 60 | RDR3 site-directed |
| | TCTGGTCGAGCA | | mutagenesis |
| C529A-F | GAGGACTTGACTCAACTGGCTGCAGGAGAAG | 61 | RDR3 site-directed |
| | ACGACACTGCTG | | mutagenesis |
| C529A-R | CAGCAGTGTCGTCTTCTCCTGCAGCCAGTTGA | 62 | RDR3 site-directed |
| | GTCAAGTCCTC | | mutagenesis |

AtCUL3A cloning

| | | | |
|---|---|---|---|
| CUL3A-F-B1 | GGGGACAAGTTTGTACAAAAAAGCAGGCTTA | 63 | CUL3A cloning |
| | ATGAGTAATCAGAAGAAGAGGAATTTTC | | |
| C3A-SR-B2 | GGGGACCACTTTGTACAAGAAAGCTGGGTATT | 64 | CUL3A cloning |
| | AGGCTAGATAGCGGTAAAGTTTCC | | |
| opCA-F4-B1 | GGGGACAAGTTTGTACAAAAAAGCAGGCTTA | 65 | opCUL3A cloning for |
| | AGCAATCAGAAGAAGCGTAACTTTC | | Duet vector |
| opCA-SR-B2 | GGGGACCACTTTGTACAAGAAAGCTGGGTATT | 66 | opCUL3A cloning for |
| | ACGCCAGATAACGATACAGTTTGC | | Duet vector |
| CUL3A-F1744 | GAAACATGAACTGAACGTGTCGACTATCGAAC | 67 | dRBX1 mutant |
| | AGGCTACAGAAATCCC | | |
| CUL3A-R1743 | GGGATTTCTGTAGCCTGTTCGATAGTCGACAC | 68 | dRBX1 mutant |
| | GTTCAGTTCATGTTTC | | |
| Nde-Myc-F4 | CATACATATGGAACAGAAACTGATCTCTGAAG | 69 | CUL3A for DUET |
| | AAG | | vector |
| Xho-opC3-R2199 | CATACTCGAGTTACGCCAGATAACGATACAGT | 70 | CUL3AforDUET |
| | TTGC | | vector |

AtRBX1 cloning

| | | | |
|---|---|---|---|
| Nco-opRBX-F4 | CATACCATGGTGGCGACCCTGGACAGCGATG | 71 | opRBX1 for DUET |
| | | | vector |
| Hin-opR1-R429 | CATAAAGCTTTTAGTGACCATACTTCTGAAAC | 72 | opRBX1 for DUET |
| | TCCCATTC | | vector |

AtUbiquitin, AtUBC8 and AtUBA1 cloning

| | | | |
|---|---|---|---|
| UBQ-F-B1 | GGGGACAAGTTTGTACAAAAAAGCAGGCTTA | 73 | Ubiquitin cloning |
| | ATGCAAATCTTCGTGAAAACACTCAC | | |
| UBQ-R-B2 | GGGGACCACTTTGTACAAGAAAGCTGGGTATT | 74 | Ubiquitin cloning |
| | AACCACCTCTTAAACGGAGAAC | | |
| Nco-HA-F4 | CATACCATGGTGTACCCATACGATGTTCCAGA | 75 | Ubiquitin cloning for |
| | TTACG | | Duet vector |
| Eco-UBQ-R231 | CATAGAATTCTTAACCACCTCTTAAACGGAGA | 76 | Ubiquitin cloning for |
| | AC | | Duet vector |
| Nco-UBC-F4 | CATACCATGGTGGCTTCGAAACGGATCTTGAA | 77 | UBC8 cloning for |
| | GGAG | | Duet vector |
| Hind-mUBC-R447 | CATAAAGCTT | 78 | UBC8 cloning for |
| | TTAGCCCATTGCATACTTCTGAGTCCAGTTTCT | | Duet vector |
| | TGCAGTAG | | |
| Eco-Duet-T7F | CATAGAATTCGAAATTAATACGACTCACTATA | 79 | UBC8 cloning for |
| | GGGGAATTG | | Duet vector |
| Duet-R1 | GATTATGCGGCCGTGTACAA | 80 | UBC8 cloning for |
| | | | Duet vector |
| Fse-UBA-F4 | CATAGGCCGGCCACCTTCACAAGCGAGCTAGT | 81 | UBA1 cloning for |
| | GAAGC | | Duet vector |
| Avr-UBA-R3243 | CATACCTAGGTCACCTGAAGTAGATAGAGACG | 82 | UBA1 cloning for |
| | AGAGG | | Duet vector |

AtWRKY54/70 cloning

| | | | |
|---|---|---|---|
| W70-F-B1 | GGGGACAAGTTTGTACAAAAAAGCAGGCTTA | 83 | WRKY70-GFP/ |
| | ATGGATACTAATAAAGCAAAAAAGC | | mCherry cloning |
| W70-R-B2 | GGGGACCACTTTGTACAAGAAAGCTGGGTAA | 84 | WRKY70-GFP/ |
| | GATAGATTCGAACATGAACTGAAG | | mCherry cloning |
| Nco-FLAG-F4 | CATACCATGGTGGACTACAAAGACGATGACG | 85 | for DUET vector |
| | ACAAA | | |
| Not-W70-R | CATAGCGGCCGCTTAAGATAGATTCGAACATG | 86 | for DUET vector |
| | AACTGAAG | | |
| W54-F-B1 | GGGGACAAGTTTGTACAAAAAAGCAGGCTTA | 87 | WRKY54-mCherry |
| | ATGGATTCGAATAGTAACAACACGAAATC | | cloning |
| W54-R-B2 | GGGGACCACTTTGTACAAGAAAGCTGGGTAC | 88 | WRKY54-mCherry |
| | ATAGCACTTGTTCTTTCATAATCAGC | | cloning |

AtBCS1, AtGSTU19, AtEDS1 and AtNIMIN1 cloning

| | | | |
|---|---|---|---|
| BC-F4-B1 | GGGGACAAGTTTGTACAAAAAAGCAGGCTTA | 89 | mCherry fusion |
| | GAAGGATCCAAGCTACTTCCTTGC | | cloning |
| BC-R1728-B2 | GGGGACCACTTTGTACAAGAAAGCTGGGTAAT | 90 | mCherry fusion |
| | GTTGTTCAACCTCCTCATGTTTGGCT | | cloning |
| G19-F4-B1 | GGGGACAAGTTTGTACAAAAAAGCAGGCTTA | 91 | mCherry fusion |
| | GCGAACGAGGTGATTCTTCTTGATTTC | | cloning |
| G19-R657-B2 | GGGGACCACTTTGTACAAGAAAGCTGGGTACT | 92 | mCherry fusion |
| | CAGGTACAAATTTCTTCCTGAGCTC | | cloning |
| EDS-F1-B1 | GGGGACAAGTTTGTACAAAAAAGCAGGCTTA | 93 | mCherry fusion |
| | ATGGCGTTTGAAGCTCTTACCGGA | | cloning |
| EDS-R1869-B2 | GGGGACCACTTTGTACAAGAAAGCTGGGTAG | 94 | mCherry fusion |
| | GTATCTGTTATTTCATCCATCATATAG | | cloning |
| Nc-FL-ED-F4 | CATACCATGGTGGACTACAAAGACGATGACG | 95 | for DUET vector |
| | ACAAAGCGTTTGAAGCTCTTACCGGAATC | | |
| Nt-ED-R1869 | CATAGCGGCCGCTTAGGTATCTGTTATTTCATC | 96 | for DUET vector |
| | CATCATATAG | | |
| NM1-F1-B1 | GGGGACAAGTTTGTACAAAAAAGCAGGCTTA | 97 | mCherry fusion |
| | ATGTATCCTAAACAATTTAGTTTATAC | | cloning |
| NM1-R426-B2 | GGGGACCACTTTGTACAAGAAAGCTGGGTAC | 98 | mCherry fusion |
| | AATGCAAGATTAAGATCTAAAGCC | | cloning |

AtPR1 promoter cloning

| | | | |
|---|---|---|---|
| BIF-pPR1F | GGGGACAAGTTTGTACAAAAAAGCAGGCTTA | 99 | dual luciferase pPR1 |
| | GTAAATTGTGTCCTATACAAAG | | reporter |
| B2R-pPR1-R2367 | GGGGACCACTTTGTACAAGAAAGCTGGGTTTT | 100 | dual luciferase pPR1 |
| | TTCTAAGTTGATAATGGTTATTG | | reporter |

mCherry-NLScloning

| | | | |
|---|---|---|---|
| mCh-F-B1 | GGGGACAAGTTTGTACAAAAAAGCAGGCTTA | 101 | mCherry-NLS cloning |
| | ATGGTGAGCAAGGGCGAGGAG | | |
| NLS-mCh-R | TTAATCTTCGACCTTGCGCTTCTTCTTAGGTGG | 102 | mCherry-NLS cloning |
| | TTCTGGTCTAGAGGATCCCTTGTACAGCTCGT | | |
| | CCATGC | | |
| B2R-NLSR | GGGGACCACTTTGTACAAGAAAGCTGGGTTTT | 103 | mCherry-NLS cloning |
| | AATCTTCGACCTTGCGCTTC | | |

VIGS cloning

| | | | |
|---|---|---|---|
| NbNPR1-TR-09 | CGACGACAAGACCCTGCAAACATTTGTGGTAA | 104 | VIGS-NbNPR1 |
| | AGCATGC | | |
| NbNPR1-TR-10 | GAGGAGAAGAGCCCTGCTACAGCATAATGGA | 105 | VIGS-NbNPR1 |
| | GAGC | | |
| Forward fusion primer for | GTGTCCCACTAACCTCAATCCCCGTTCTCCC | 106 | VIGS-NbCUL3 |
| Niben101Scf01326g05017 | | | |
| & | | | |
| Niben101Scf06545g03015 | | | |
| Reverse fusion primer for | GGATTGAGGTTAGTGGGACACTCTTTCAATCT | 107 | VIGS-NbCUL3 |
| Niben001Scf01326g05017 | C | | |
| & | | | |
| Niben101Scf06545g03015 | | | |
| LIC1 for | CGACGACAAGACCCTAGACCATATCATCCACT | 108 | VIGS-NbCUL3 |
| Niben001Scf01326g05017 | CTAGC | | |
| LIC2 for | GAGGAGAAGAGCCCTGTGCATTGCATCCTGAG | 109 | VIGS-NbCUL3 |
| Niben001Scf06545g03015 | AAGTTTTC | | |
| LIC1 for | CGACGACAAGACCCTGCAAACATTTGTGGTAA | 110 | VIGS-NbCUL3 |
| Niben101Scf14780g01001.1 | AGCATGC | | |
| LIC2 for | GAGGAGAAGAGCCCTGCTACAGCATAATGGA | ill | VIGS-NbCUL3 |
| Niben101Scf14780g01001.1 | GAGC | | |
| NbeIF4A-QF | GCTTTGGTCTTGGCACCTACTC | 112 | qPCRon VIGS |
| NbeIF4A-QR | TGCTCGCATGACCTTTTCAA | 113 | qPCRon VIGS |
| NbCUL3-1-QF | GCCAACACAGTCCGTCACTA | 114 | qPCRon VIGS |
| NbCUL3-1-QR | TTATCGCAGACCCCCAAA | 115 | qPCRon VIGS |
| NbCUL3-2-QF | TGTGGATCCGAAATATGCTG | 116 | qPCRon VIGS |
| NbCUL3-2-QR | AACTAAGACCACTAGCGTTATGATTG | 117 | qPCRon VIGS |
| NbCUL3-3-QF | TGAGTACGCTTCTTGGACTGAT | 118 | qPCRon VIGS |
| NbCUL3-3-QR | TTCTCATCAGTCCCCGGTTA | 119 | qPCRon VIGS |
| NbCUL3-4-QF | TTCTTGGAAGAGCTGAATAGGC | 120 | qPCRon VIGS |
| NbCUL3-4-QR | TCAGAATATCCCGTATCATTTGC | 121 | qPCRon VIGS |
| NbNPR1a-QF | TGTGTGTGTTTGTGTGGACAAT | 122 | qPCRon VIGS |
| NbNPR1a-QR | GAACGCTACAGCTGGCCTAC | 123 | qPCRon VIGS |
| NbNPR1b-QF | CTTCGAAGGTTCGGTTATGC | 124 | qPCRon VIGS |
| NbNPR1b-QR | GCTTCTCCTAGCAGTGGATCTC | 125 | qPCRon VIGS |
| NbPR1-QF | GTGCCCAAAATTCTCAACAAG | 126 | qPCRon VIGS |
| NbPR1-QR | TTCTACACCTACATCTGCACGAG | 127 | qPCRon VIGS |

Genotyping of npr1-2, wrky54 and wrky70 mutations

| | | | |
|---|---|---|---|
| npr1-2-LP | GATGATTTCTACAGCGACGC | 128 | npr1-2 mutant |
| | | | genotyping |
| npr1-2-RP | CCATAGCTTAATGCAGATGG | 129 | npr1-2 mutant |
| | | | genotyping |
| WRKY54-LP | TTCTTGATTCTTTTGATCCCG | 130 | wrky54 |
| | | | (SALK_017254) |
| | | | mutant genotyping |
| WRKY54-RP | GCTGGTGTTGTTCTCTTGCTC | 131 | wrky54 |
| | | | (SALK_017254) |
| | | | mutant genotyping |
| WRKY70-LP | TGATCTTCGGAATCCATGAAG | 132 | wrky70 |
| | | | (SALK_025198) |
| | | | mutant genotyping |
| WRKY70-RP | CAAACCACACCAAGAGGAAAG | 133 | wrky70 |
| | | | (SALK_025198) |
| | | | mutant genotyping |

TABLE 7
Key Resources Table.

| | | |
|---|---|---|
| REAGENT or RESOURCE | SOURCE | IDENTIFIER |

Antibodies

| | | |
|---|---|---|
| Mouse monoclonal anti-GFP (JL8) | Clontech | Cat. #632381; |
| | | RRID: AB_2313808 |
| Mouse monoclonal anti-RFP (6G6) | Chromotek | Cat. #6g6-20 |
| Mouse monoclonal anti-Ubiquitin (P4D1) | Santa Cruz | Cat. #sc-8017; |
| | | RRID: AB_2762364 |
| Mouse monoclonal anti-HA | BioLegend | Cat. #901502; |
| | | RRID: AB_2565007 |
| Alpaca anti-GFP coupled to agarose beads (GFP- Trap_A) | Chromotek | Cat. #gta-10 |
| Alpaca anti-RFP coupled to agarose beads (RFP- Trap_A) | Chromotek | Cat. #rta-10 |
| Pierce ™ anti-HA coupled Magnetic Beads | ThermoFisher | Cat. #88837 |
| Pierce ™ anti-DYKDDDDK coupled Magnetic Agarose | ThermoFisher | Cat. #A36797 |
| Pierce ™ Glutathione Magnetic Agarose Beads | ThermoFisher | Cat. #78601 |
| Mouse monoclonal anti-Baculovirus Envelope gp64 (AcV5) | ThermoFisher | Cat. #14-6995-82; |
| | | RRID: AB 468384 |
| Rabbit polyclonal anti-AtCUL3A | PhytoAB, | Cat. #PHY0978S |
| | Dieterle et al. | |
| | (2005) | |
| Rabbit polyclonal anti-AtEDS1 | Agrisera | Cat. #AS13-2751 |
| Rabbit polyclonal anti-AtNIMIN1 (E2374) | ABclonal | Cat. #WG-01110D |
| Rabbit polyclonal anti-AtNPR1 | Mou et al., 2003 | N/A |
| Mouse monoclonal anti-c-Myc (9E10) | Santa Cruz | Cat. #sc-40; |
| | | RRID: AB_627268 |
| Direct-Blot ™ HRP anti-DYKDDDDK Tag Antibody (anti- | BioLegend | Cat. #637311; |
| FLAG) | | RRID: AB_2566706 |
| Mouse monoclonal anti-β-Tubulin (F-1) | Santa Cruz | Cat. #sc-166729; |
| | | RRID: AB_2010699 |
| Rabbit polyclonal anti-Histone H3 | Agrisera | Cat. #AS10-710 |
| Rabbit polyclonal anti-Actin | Agrisera | Cat. #AS13-2640 |
| Anti-GST Antibody, HRP Conjugated | GE Healthcare | Cat. #RPN1236; |
| | | RRID: AB_771429 |

Bacterial and Virus Strains

| | | |
|---|---|---|
| Pseudomonas syringae pv. maculicola ES4326/AvrRpt2 | Cao et al., 1994 | N/A |
| Pseudomonas syringae pv. maculicola ES4326/AvrRpm1 | Reuber and | N/A |
| | Ausbel, 1996 | |
| Pseudomonas fluorescens Pf0-1/AvrRps4 | Sohn et al., 2012 | N/A |
| Pseudomonas fluorescens Pf0-1/AvrRps4^{KRVY-AAAA} | Sohn et al., 2012 | N/A |
| Agrobacterium tumefaciens, strain GV3101 | N/A | N/A |
| Escherichia coli (E. coli), strain BL21(DE3) | NEB | Cat. #C2527H |

Chemicals, Peptides, and Recombinant Proteins

| | | |
|---|---|---|
| Sodium salicylate | Sigma-Aldrich | Cat. #S3007; |
| | | CAS: 54-21-7 |
| Dexamethasone | Sigma-Aldrich | Cat. #D1756-25MG; |
| | | CAS: 50-02-2 |
| β-Estradiol | Sigma-Aldrich | Cat. #E2758; |
| | | CAS: 50-28-2 |
| Methyl viologen dichloride hydrate (MV) | Sigma-Aldrich | Cat. #856177; |
| | | CAS: 75365-73-0 |
| Deubiquitinase (DUB) inhibitor (PR-619) | Life Sensors | Cat. #SI9619; |
| | | CAS: 2645-32-1 |
| N-Ethylmaleimide (NEM) | Sigma-Aldrich | Cat. #E3876; |
| | | CAS: 128-53-0 |

Critical Commercial Assays

| | | |
|---|---|---|
| Pierce ™ Silver Stain Kit | ThermoFisher | Cat. #24612 |
| Dual-Luciferase Reporter Assay System | Promega | Cat. #E1910 |
| QuikChange II Site-Directed Mutagenesis Kit | Agilent | Cat. #200524 |
| FastStart Universal SYBR Green Master Kit | Roche | Cat. #04913850001 |

Experimental Models: Organisms/Strains

| | | |
|---|---|---|
| Arabidopsis: npr1-2 | Cao et al., 1997 | N/A |
| Arabidopsis: sid2-2 | Wildermuth | N/A |
| | et al., 2001 | |
| Arabidopsis: NPR1-GFP/npr1-2 | Mou et al., 2003 | N/A |
| Arabidopsis: npr1^{sim3}-GFP/npr1-2 | Saleh et al., 2015 | N/A |
| Arabidopsis: npr1^{□BTB}-GFP/npr1-2 | This paper | N/A |
| Arabidopsis: npr1^{sim3}-GFP/Col-0 | This paper | N/A |
| Arabidopsis: npr1^{rdr1}-GFP/npr1-2 | This paper | N/A |
| Arabidopsis: npr1^{rdr2}-GFP/npr1-2 | This paper | N/A |
| Arabidopsis: npr1^{rdr3}-GFP/npr1-2 | This paper | N/A |
| Arabidopsis: dex: AvrRpt2/Col-0 | McNellis | N/A |
| | et al., 1998 | |
| Arabidopsis: dex: AvrRpt2/rps2 | Gu et al., 2016 | N/A |
| Arabidopsis: dex: AvrRpt2/npr1-2 | This paper | N/A |

Oligonucleotides

| | | |
|---|---|---|
| Primers used in this study are listed in Table 6 | This paper | N/A |

Recombinant DNA

| | | |
|---|---|---|
| pTRV2-NbCUL3/NbNPR1 | This paper | N/A |
| pEG203-CUL3/cul3^{□DRBX1} | This paper | N/A |
| pK7WGF2-CUL3 | This paper | N/A |
| pEG204-Ubiquitin | This paper | N/A |
| pEG100-mCherry-ATG8/NBR1/Ubiquitin/NLS | This paper | N/A |
| pSITE-4NB-EDS1/GSTU19/BCS1/NIMIN1 | This paper | N/A |
| pSITE-cEYFP-N1-NPR1 (NPR1-YC, WT and mutant | This paper | N/A |
| variants) | | |
| pSITE-nEYFP-C1-NPR1 (YN-NPR1) | This paper | N/A |
| pSITE-cEYFP-N1-NPR1/2/3/4/5/6 (NPR1/2/3/4/5/6-YC) | This paper | N/A |
| pSITE-nEYFP-C1-CUL3 (YN-CUL3) | This paper | N/A |
| pK7FWG2-NPR1 (WT, mutant and truncation variants) | This paper, | N/A |
| | Saleh et al. | |
| | (2015) | |
| pK7FWG2-NPR2/3/4/5/6 | This paper | N/A |
| pLN462-NPR1 (WT, mutant and truncation variants) | This paper | N/A |
| pLN462-NPR2/3/4/5/6 | This paper | N/A |
| pETDuet-1: FLAG-WRKY70 + GST-NPR1/GST- | This paper | N/A |
| npr1^{sim3}/GST | | |
| pETDuet-1: FLAG-EDS1 + GST-NPR1/GST | This paper | N/A |
| pCDFDuet-1: HA-Ub + UBC8 + UBA1 | This paper | N/A |
| pACYCDuet-1: RBX1 + Myc-CUL3 | This paper | N/A |
| pCDFDuet-1: Myc-CUL3 | This paper | N/A |
| pDEST15-NPR1 (WT, mutant and truncation variants) | This paper, | N/A |
| | Saleh et al. | |
| | (2015) | |

Software and Algorithms

| | | |
|---|---|---|
| ImageJ (FIJI) (v2.0.0-rc-69/1.52p) | Schindelin et al., 2012 | https://imagej.nih.go v/ij/ |
| Scaffold (v4.8.2) | Proteome Software | N/A |
| R (v3.5.3) | N/A | N/A |
| Prism 8 | GraphPad | https://www.graphpad.com/ |
| ShinyGO v0.61 | Ge et al., 2019 | http://bioinformatics.sdstate.edu/go/ |
| UpSet plots | Khan and Mathelier, | https://asntech.shinyapps.io/intervene/ |
| | 2017 | |
| IUPred2a | Meszaros et al., 2018 | http://iupred.elte.hu/ |

Other

| | | |
|---|---|---|
| Orion Star ™ A222 Portable | ThermoFisher | Cat. #STARA2220 |
| Conductivity Meter with 4- | | (meter); #013005MD |
| Electrode Conductivity Cell | | (probe) |

Banani, S. F., Lee, H. O., Hyman, A. A., and Rosen, M. K. (2017). Biomolecular condensates: organizers of cellular biochemistry. Nat Rev Mol Cell Biol 18, 285-298.
Bartsch, M., Gobbato, E., Bednarek, P., Debey, S., Schultze, J. L., Bautor, J., and Parker, J. E. (2006). Salicylic acid-independent ENHANCED DISEASE SUSCEPTIBILITY1 signaling in Arabidopsis immunity and cell death is regulated by the monooxygenase FMO1 and the Nudix hydrolase NUDT7. The Plant cell 18, 1038-1051.
Bolte, S., and Cordelieres, F. P. (2006). A guided tour into subcellular colocalization analysis in light microscopy. J Microsc 224, 213-232.
Bouchard, J. J., Otero, J. H., Scott, D. C., Szulc, E., Martin, E. W., Sabri, N., Granata, D., Marzahn, M. R., Lindorff-Larsen, K., Salvatella, X., et al. (2018). Cancer Mutations of the Tumor Suppressor SPOP Disrupt the Formation of Active, Phase-Separated Compartments. Mol Cell 72, 19-36 e18.
Brkljacic, J., Zhao, Q., and Meier, I. (2009). WPP-domain proteins mimic the activity of the HSC70-1 chaperone in preventing mistargeting of RanGAP1-anchoring protein WIT1. Plant physiology 151, 142-154.
Bruggeman, Q., Raynaud, C., Benhamed, M., and Delarue, M. (2015). To die or not to die? Lessons from lesion mimic mutants. Frontiers in plant science 6, 24.
Cao, H., Bowling, S. A., Gordon, A. S., and Dong, X. N. (1994). Characterization of an Arabidopsis Mutant That Is Nonresponsive to Inducers of Systemic Acquired-Resistance. The Plant cell 6, 1583-1592.
Cao, H., Glazebrook, J., Clarke, J. D., Volko, S., and Dong, X. (1997). The Arabidopsis NPR1 gene that controls systemic acquired resistance encodes a novel protein containing ankyrin repeats. Cell 88, 57-63.
Chahtane, H., Zhang, B., Norberg, M., LeMasson, M., Thevenon, E., Bako, L., Benlloch, R., Holmlund, M., Parcy, F., Nilsson, O., et al. (2018). LEAFY activity is post-transcriptionally regulated by BLADE ON PETIOLE2 and CULLIN3 in Arabidopsis. New Phytol 220, 579-592.
Chang, M., Zhao, J., Chen, H., Li, G., Chen, J., Li, M., Palmer, I. A., Song, J., Alfano, J. R., Liu, F., et al. (2019). PBS3 Protects EDS1 from Proteasome-Mediated Degradation in Plant Immunity. Mol Plant 12, 678-688.
Clarke, S. M., Mur, L. A., Wood, J. E., and Scott, I. M. (2004). Salicylic acid dependent signaling promotes basal thermotolerance but is not essential for acquired thermotolerance in Arabidopsis thaliana. Plant J 38, 432-447.
Clough, S. J., and Bent, A. F. (1998). Floral dip: a simplified method for Agrobacterium-mediated transformation of Arabidopsis thaliana. Plant J 16, 735-743.
Devadas, S. K., Enyedi, A., and Raina, R. (2002). The Arabidopsis hrl1 mutation reveals novel overlapping roles for salicylic acid, jasmonic acid and ethylene signalling in cell death and defence against pathogens. Plant J 30, 467-480.
Dieterle, M., Thomann, A., Renou, J. P., Parmentier, Y., Cognat, V., Lemonnier, G., Muller, R., Shen, W. H., Kretsch, T., and Genschik, P. (2005). Molecular and functional characterization of Arabidopsis Cullin 3A. Plant J 41, 386-399.
Ding, Y., Dommel, M., and Mou, Z. (2016). Abscisic acid promotes proteasome-mediated degradation of the transcription coactivator NPR1 in Arabidopsis thaliana. Plant J 86, 20-34.
Dong, Y., Burch-Smith, T. M., Liu, Y., Mamillapalli, P., and Dinesh-Kumar, S. P. (2007). A ligation-independent cloning tobacco rattle virus vector for high-throughput virus-induced gene silencing identifies roles for NbMADS4-1 and -2 in floral development. Plant physiology 145, 1161-1170.
Erdos, G., Meszaros, B., Reichmann, D., and Dosztanyi, Z. (2019). Large-Scale Analysis of Redox-Sensitive Conditionally Disordered Protein Regions Reveals Their Widespread Nature and Key Roles in High-Level Eukaryotic Processes. Proteomics 19, e1800070.
Fernandez-Pozo, N., Menda, N., Edwards, J. D., Saha, S., Tecle, I. Y., Strickler, S. R., Bombarely, A., Fisher-York, T., Pujar, A., Foerster, H., et al. (2015a). The Sol Genomics Network (SGN)—from genotype to phenotype to breeding. Nucleic acids research 43, D1036-1041.
Fernandez-Pozo, N., Rosli, H. G., Martin, G. B., and Mueller, L. A. (2015b). The SGN VIGS tool: user-friendly software to design virus-induced gene silencing (VIGS) constructs for functional genomics. Mol Plant 8, 486-488.
Fu, Z. Q., and Dong, X. (2013). Systemic acquired resistance: turning local infection into global defense. Annual review of plant biology 64, 839-863.
Fu, Z. Q., Yan, S., Saleh, A., Wang, W., Ruble, J., Oka, N., Mohan, R., Spoel, S. H., Tada, Y., Zheng, N., et al. (2012). NPR3 and NPR4 are receptors for the immune signal salicylic acid in plants. Nature 486, 228-232.
Furukawa, M., He, Y. J., Borchers, C., and Xiong, Y. (2003). Targeting of protein ubiquitination by BTB-Cullin 3-Roc1 ubiquitin ligases. Nat Cell Biol 5, 1001-1007.
Ge, S. X., Jung, D., and Yao, R. (2019). ShinyGO: a graphical enrichment tool for animals and plants. Bioinformatics.
Geyer, R., Wee, S., Anderson, S., Yates, J., and Wolf, D. A. (2003). BTB/POZ domain proteins are putative substrate adaptors for cullin 3 ubiquitin ligases. Mol Cell 12, 783-790.
Gomez-Diaz, C., and Ikeda, F. (2018). Roles of ubiquitin in autophagy and cell death. Semin Cell Dev Biol.
Gu, Y., Zebell, S. G., Liang, Z., Wang, S., Kang, B. H., and Dong, X. (2016). Nuclear Pore Permeabilization Is a Convergent Signaling Event in Effector-Triggered Immunity. Cell 166, 1526-1538 e1511.
Haynes, C., Oldfield, Ji, F., Klitgord, N., Cusick, M. E., Radivojac, P., Uversky, V. N., Vidal, M., and Iakoucheva, L. M. (2006). Intrinsic disorder is a common feature of hub proteins from four eukaryotic interactomes. PLoS Comput Biol 2, e100.
Heidrich, K., Wirthmueller, L., Tasset, C., Pouzet, C., Deslandes, L., and Parker, J. E. (2011). Arabidopsis EDS1 connects pathogen effector recognition to cell compartment-specific immune responses. Science (New York, N.Y. 334, 1401-1404.
Jakob, U., Kriwacki, R., and Uversky, V. N. (2014). Conditionally and transiently disordered proteins: awakening cryptic disorder to regulate protein function. Chem Rev 114, 6779-6805.
Jin, H., Choi, S. M., Kang, M. J., Yun, S. H., Kwon, D. J., Noh, Y. S., and Noh, B. (2018). Salicylic acid-induced transcriptional reprogramming by the HAC-NPR1-TGA histone acetyltransferase complex in Arabidopsis. Nucleic acids research 46, 11712-11725.
Jones, J. D., and Dangl, J. L. (2006). The plant immune system. Nature 444, 323-329.
Khan, A., and Mathelier, A. (2017). Intervene: a tool for intersection and visualization of multiple gene or genomic region sets. BMC Bioinformatics 18, 287.
Kinkema, M., Fan, W., and Dong, X. (2000). Nuclear localization of NPR1 is required for activation of PR gene expression. The Plant cell 12, 2339-2350.
Knoth, C., Ringler, J., Dangl, J. L., and Eulgem, T. (2007). Arabidopsis WRKY70 is required for full RPP4-mediated disease resistance and basal defense against Hyaloperonospora parasitica. Mol Plant Microbe Interact 20, 120-128.
Lai, Y. S., Renna, L., Yarema, J., Ruberti, C., He, S. Y., and Brandizzi, F. (2018). Salicylic acid-independent role of NPR1 is required for protection from proteotoxic stress in the plant endoplasmic reticulum. Proceedings of the National Academy of Sciences of the United States of America 115, E5203-E5212.
Levin-Kravets, O., Tanner, N., Shohat, N., Attali, I., Keren-Kaplan, T., Shusterman, A., Artzi, S., Varvak, A., Reshef, Y., Shi, X., et al. (2016). A bacterial genetic selection system for ubiquitylation cascade discovery. Nature methods 13, 945-952.
Li, M., Chen, H., Chen, J., Chang, M., Palmer, I. A., Gassmann, W., Liu, F., and Fu, Z. Q. (2018). TCP Transcription Factors Interact With NPR1 and Contribute Redundantly to Systemic Acquired Resistance. Frontiers in plant science 9, 1153.
Liu, L., Sonbol, F. M., Huot, B., Gu, Y., Withers, J., Mwimba, M., Yao, J., He, S. Y., and Dong, X. (2016). Salicylic acid receptors activate jasmonic acid signalling through a non-canonical pathway to promote effector-triggered immunity. Nat Commun 7, 13099.
Liu, Y., Schiff, M., Czymmek, K., Talloczy, Z., Levine, B., and Dinesh-Kumar, S. P. (2005). Autophagy regulates programmed cell death during the plant innate immune response. Cell 121, 567-577.
Liu, Y., Schiff, M., Marathe, R., and Dinesh-Kumar, S. P. (2002). Tobacco Rarl, EDS1 and NPR1/NIM1 like genes are required for N-mediated resistance to tobacco mosaic virus. Plant J 30, 415-429.
Maier, F., Zwicker, S., Huckelhoven, A., Meissner, M., Funk, J , Pfitzner, A. J., and Pfitzner, U. M. (2011). NONEXPRESSOR OF PATHOGENESIS-RELATED PROTEINS1 (NPR1) and some NPR1-related proteins are sensitive to salicylic acid. Molecular plant pathology 12, 73-91.
Mao, P., Duan, M., Wei, C., and Li, Y. (2007). WRKY62 transcription factor acts downstream of cytosolic NPR1 and negatively regulates jasmonate-responsive gene expression. Plant Cell Physiol 48, 833-842.
McNellis, T. W., Mudgett, M. B., Li, K., Aoyama, T., Horvath, D., Chua, N. H., and Staskawicz, B. J. (1998). Glucocorticoid-inducible expression of a bacterial avirulence gene in transgenic Arabidopsis induces hypersensitive cell death. Plant J 14, 247-257.
Meszaros, B., Erdos, G., and Dosztanyi, Z. (2018). IUPred2A: context-dependent prediction of protein disorder as a function of redox state and protein binding. Nucleic acids research 46, W329-W337.
Mindrinos, M., Katagiri, F., Yu, G. L., and Ausubel, F. M. (1994). The A. thaliana disease resistance gene RPS2 encodes a protein containing a nucleotide-binding site and leucine-rich repeats. Cell 78, 1089-1099.
Mou, Z., Fan, W., and Dong, X. (2003). Inducers of plant systemic acquired resistance regulate NPR1 function through redox changes. Cell 113, 935-944.
Munch, D., Rodriguez, E., Bressendorff, S., Park, O. K., Hofius, D., and Petersen, M. (2014). Autophagy deficiency leads to accumulation of ubiquitinated proteins, ER stress, and cell death in Arabidopsis. Autophagy 10, 1579-1587.
Nelson, B. K., Cai, X., and Nebenfuhr, A. (2007). A multicolored set of in vivo organelle markers for co-localization studies in Arabidopsis and other plants. Plant J 51, 1126-1136.
Olate, E., Jimenez-Gomez, J. M., Holuigue, L., and Salinas, J. (2018). NPR1 mediates a novel regulatory pathway in cold acclimation by interacting with HSFA1 factors. Nat Plants 4, 811-823.
Pike, S. M., Zhang, X. C., and Gassmann, W. (2005). Electrophysiological characterization of the Arabidopsis avrRpt2-specific hypersensitive response in the absence of other bacterial signals. Plant physiology 138, 1009-1017.
Rate, D. N., and Greenberg, J. T. (2001). The Arabidopsis aberrant growth and death2 mutant shows resistance to Pseudomonas syringae and reveals a role for NPR1 in suppressing hypersensitive cell death. Plant J 27, 203-211.
Reichmann, D , Xu, Y., Cremers, C. M., Ilbert, M., Mittelman, R., Fitzgerald, M. C., and Jakob, U. (2012). Order out of disorder: working cycle of an intrinsically unfolded chaperone. Cell 148, 947-957.
Reuber, T. L., and Ausubel, F. M. (1996). Isolation of Arabidopsis genes that differentiate between resistance responses mediated by the RPS2 and RPM1 disease resistance genes. The Plant cell 8, 241-249.
Rodriguez, E., El Ghoul, H., Mundy, J., and Petersen, M. (2016). Making sense of plant autoimmunity and ‘negative regulators’. FEBS J 283, 1385-1391.
Saleh, A., Withers, J., Mohan, R., Marques, J., Gu, Y., Yan, S., Zavaliev, R., Nomoto, M., Tada, Y., and Dong, X. (2015). Posttranslational Modifications of the Master Transcriptional Regulator NPR1 Enable Dynamic but Tight Control of Plant Immune Responses. Cell Host Microbe 18, 169-182.
Sappl, P. G., Onate-Sanchez, L., Singh, K. B., and Millar, A. H. (2004). Proteomic analysis of glutathione S-transferases of Arabidopsis thaliana reveals differential salicylic acid-induced expression of the plant-specific phi and tau classes. Plant molecular biology 54, 205-219.
Sarris, P. F., Duxbury, Z., Huh, S. U., Ma, Y., Segonzac, C., Sklenar, J., Derbyshire, P., Cevik, V., Rallapalli, G., Saucet, S. B., et al. (2015). A Plant Immune Receptor Detects Pathogen Effectors that Target WRKY Transcription Factors. Cell 161, 1089-1100.
Schindelin, J., Arganda-Carreras, I., Frise, E., Kaynig, V., Longair, M., Pietzsch, T., Preibisch, S., Rueden, C., Saalfeld, S., Schmid, B., et al. (2012). Fiji: an open-source platform for biological-image analysis. Nature methods 9, 676-682.
Shimada, S., Komatsu, T., Yamagami, A., Nakazawa, M., Matsui, M., Kawaide, H., Natsume, M., Osada, H., Asami, T., and Nakano, T. (2015). Formation and dissociation of the BSS1 protein complex regulates plant development via brassinosteroid signaling. The Plant cell 27, 375-390.
Silva, K. J. P., Mahna, N., Mou, Z., and Folta, K. M. (2018). NPR1 as a transgenic crop protection strategy in horticultural species. Hortic Res 5, 15.
Sohn, K. H., Hughes, R. K., Piquerez, S. J., Jones, J. D., and Banfield, M. J. (2012). Distinct regions of the Pseudomonas syringae coiled-coil effector AvrRps4 are required for activation of immunity. Proceedings of the National Academy of Sciences of the United States of America 109, 16371-16376.
Spoel, S. H., Koornneef, A., Claessens, S. M., Korzelius, J. P., Van Pelt, J. A., Mueller, M. J., Buchala, A. J., Metraux, J. P., Brown, R., Kazan, K., et al. (2003). NPR1 modulates cross-talk between salicylate- and jasmonate-dependent defense pathways through a novel function in the cytosol. The Plant cell 15, 760-770.
Spoel, S. H., Mou, Z., Tada, Y., Spivey, N. W., Genschik, P., and Dong, X. (2009). Proteasome-mediated turnover of the transcription coactivator NPR1 plays dual roles in regulating plant immunity. Cell 137, 860-872.
Tada, Y., Spoel, S. H., Pajerowska-Mukhtar, K., Mou, Z., Song, J., Wang, C., Zuo, J., and Dong, X. (2008). Plant immunity requires conformational changes of NPR1 via S-nitrosylation and thioredoxins. Science (New York, N.Y. 321, 952-956.
Tsuda, K., Mine, A., Bethke, G., Igarashi, D., Botanga, C. J., Tsuda, Y., Glazebrook, J., Sato, M., and Katagiri, F. (2013). Dual regulation of gene expression mediated by extended MAPK activation and salicylic acid contributes to robust innate immunity in Arabidopsis thaliana. PLoS genetics 9, e1004015.
Ustun, S., Hafren, A., and Hofius, D. (2017). Autophagy as a mediator of life and death in plants. Current opinion in plant biology 40, 122-130.
Wang, D., Amornsiripanitch, N., and Dong, X. (2006). A genomic approach to identify regulatory nodes in the transcriptional network of systemic acquired resistance in plants. PLoS pathogens 2, e 123.
Wang, D., Weaver, N. D., Kesarwani, M., and Dong, X. (2005). Induction of protein secretory pathway is required for systemic acquired resistance. Science (New York, N.Y. 308, 1036-1040.
Wang, W., Ye, R., Xin, Y., Fang, X., Li, C., Shi, H., Zhou, X., and Qi, Y. (2011). An importin beta protein negatively regulates MicroRNA activity in Arabidopsis. The Plant cell 23, 3565-3576.
Wiermer, M., Feys, B. J., and Parker, J. E. (2005). Plant immunity: the EDS1 regulatory node. Current opinion in plant biology 8, 383-389.
Wildermuth, M. C., Dewdney, J., Wu, G., and Ausubel, F. M. (2001). Isochorismate synthase is required to synthesize salicylic acid for plant defence. Nature 414, 562-565.
Xu, G., Greene, G. H., Yoo, H., Liu, L., Marques, J., Motley, J., and Dong, X. (2017a). Global translational reprogramming is a fundamental layer of immune regulation in plants. Nature 545, 487-490.
Xu, G., Yuan, M., Ai, C., Liu, L., Zhuang, E., Karapetyan, S., Wang, S., and Dong, X. (2017b). uORF-mediated translation allows engineered plant disease resistance without fitness costs. Nature 545, 491-494.
Xu, L., Wei, Y., Reboul, J., Vaglio, P., Shin, T. H., Vidal, M., Elledge, S. J., and Harper, J. W. (2003). BTB proteins are substrate-specific adaptors in an SCF-like modular ubiquitin ligase containing CUL-3. Nature 425, 316-321.
Yoshimoto, K., Jikumaru, Y., Kamiya, Y., Kusano, M., Consonni, C., Panstruga, R., Ohsumi, Y., and Shirasu, K. (2009). Autophagy negatively regulates cell death by controlling NPR1-dependent salicylic acid signaling during senescence and the innate immune response in Arabidopsis. The Plant cell 21, 2914-2927.
Zavaliev, R., and Epel, B. L. (2015). Imaging Callose at Plasmodesmata Using Aniline Blue: Quantitative Confocal Microscopy. In Plasmodesmata: Methods and Protocols (ed. M. Heinlein). Methods in Molecular Biology 1217, 105-119.
Zhang, B., Holmlund, M., Lorrain, S., Norberg, M., Bako, L., Fankhauser, C., and Nilsson, O. (2017). BLADE-ON-PETIOLE proteins act in an E3 ubiquitin ligase complex to regulate PHYTOCHROME INTERACTING FACTOR 4 abundance. Elife 6.
Zhang, B., Van Aken, O., Thatcher, L., De Clercq, I., Duncan, O., Law, S. R., Murcha, M. W., van der Merwe, M., Seifi, H. S., Carrie, C., et al. (2014). The mitochondrial outer membrane AAA ATPase AtOM66 affects cell death and pathogen resistance in Arabidopsis thaliana. Plant J 80, 709-727.
Zhang, Y., Yang, Y., Fang, B., Gannon, P., Ding, P., Li, X., and Zhang, Y. (2010). Arabidopsis snc2-1D activates receptor-like protein-mediated immunity transduced through WRKY70. The Plant cell 22, 3153-3163.
Zhou, J., Spallek, T., Faulkner, C., and Robatzek, S. (2012). CalloseMeasurer: a novel software solution to measure callose deposition and recognize spreading callose patterns. Plant Methods 8, 49.
Zhou, J., Wang, J., Cheng, Y., Chi, Y. J., Fan, B., Yu, J. Q., and Chen, Z. (2013). NBR1-mediated selective autophagy targets insoluble ubiquitinated protein aggregates in plant stress responses. PLoS genetics 9, e1003196.
Zhou, J., Zhang, Y., Qi, J., Chi, Y., Fan, B., Yu, J. Q., and Chen, Z. (2014). E3 ubiquitin ligase CHIP and NBR1-mediated selective autophagy protect additively against proteotoxicity in plant stress responses. PLoS genetics 10, e1004116.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A nucleic acid encoding a npr1 protein, wherein the npr1 protein forms salicylic acid-independent NPR1 condensates.

2. (canceled)

3. The nucleic acid of claim 1, wherein the nucleic acid is operably linked to one or more expression control elements selected from the group consisting of: a promoter, one or more upstream open reading frames (uORFs), or the promoter and the one or more uORFs.

4. The nucleic acid of claim 3, wherein the promoter is selected from the group consisting of: a constitutive promoter, an inducible promoter, a temporally-regulated promoter, a developmentally regulated promoter, a chemically regulated promoter, a tissue-preferred promoter, a tissue-specific promoter, a TBF1 promoter, a 35S promoter, a ubiquitin promoter, a tCUP cryptic constitutive promoter, a Rsyn7 promoter, a pathogen-inducible promoter, a maize In2-2 promoter, a tobacco PR-1a promoter, a glucocorticoid-inducible promoter, an estrogen-inducible promoter, a tetracycline-inducible promoter, a tetracycline-repressible promoter, a T3 promoter, a T7 promoter, and a SP6 promoter.

5. (canceled)

6. The nucleic acid of claim 3, wherein the uORF comprises a TBF1 gene uORF.

7. The nucleic acid of claim 5, wherein the expression control elements comprise the TBF1 promoter and one or more TBF1 uORFs.

8. (canceled)

9. The nucleic acid of claim 1, wherein the npr1 protein comprises one or more mutations in at least one redox-sensitive intrinsically disordered region (RDR), wherein the one or more mutations reduce the redox-sensitivity of the RDR.

10. The nucleic acid of claim 9, wherein the one or more mutations comprises a substitution of one or more cysteines in at least one RDR, a deletion of one or more cysteines in at least one RDR, or a combination thereof.

11. The nucleic acid of claim 10, wherein the one or more cysteines are located in a region corresponding to residues 140-160, 368-404, or 510-539 of SEQ ID NO: 1.

12. (canceled)

13. The nucleic acid of claim 9, wherein the npr1 protein has increased interaction with CUL3 compared to wild-type NPR1 in the absence of salicylic acid.

14. (canceled)

15. The nucleic acid of claim 11, wherein the npr1 protein comprises a mutation of a cysteine corresponding to the cysteine at position 378, of SEQ ID NO: 1, position 385 of SEQ ID NO: 1, position 394 of SEQ ID NO: 1, positions 378 and 385 of SEQ ID NO: 1, positions 378 and 394 of SEQ ID NO: 1, positions 385 and 394 of SEQ ID NO: 1, or positions 378, 385, and 394 of SEQ ID NO: 1, or positions 150, 155, 156, and 160 of SEQ ID NO: 1.

16. (canceled)

17. The nucleic acid of claim 15, wherein the mutation of the cysteine comprises an alanine substitution.

18. (canceled)

19. The nucleic acid of claim 1, wherein the nucleic acid encodes a ΔCTD npr1 ΔCTD protein, a BTB domain npr1 protein, or a sim3 npr1 protein.

20. The nucleic acid of claim 1, where the nucleic acid encodes:
(a) a protein comprising the amino acid sequence of any of SEQ ID NOS: 134-160 or an ortholog thereof; or
(b) a protein having at least 70% identity to a protein comprising the amino acid sequence of any of SEQ ID NOS: 134-160.

21. (canceled)

22. A plant or plant cell expressing the nucleic acid of claim 1.

23. (canceled)

24. The plant or plant cell of claim 22, wherein the plant is a food crop plant, a biofuel plant, a corn plant, a legume plant, a bean plant, a rice plant, a soybean plant, a cotton plant, a sugarcane plant, a tobacco plant, a palm oil plant, a date palm, a wheat, a vegetable plant, a squash plant, a Solanaceae plant, a tomato, a banana plant, a potato plant, a pepper plant, a moss plant, a parsley plant, a sunflower plant, a mustard plant, a sorghum plant, a millet plant, a citrus plant, an apple plant, a strawberry plant, a rapeseed plant, a cabbage plant, a cassava plant, a coffee plant, a sweet potato plant, a jatropha plant, or a switchgrass plant.

25. (canceled)

26. (canceled)

27. A method of increasing stress tolerance in a plant, comprising expressing in the plant the nucleic acid of claim 1.

28. The method of claim 27, wherein the stress is biotic or abiotic stress.

29. The method of claim 28, wherein the biotic stress comprises pathogen infection.

30. (canceled)

31. The method of claim 28, wherein the abiotic stress comprises high temperature (heat shock) stress, low temperature (cold shock) stress, oxidative stress, or DNA damage.

32. The method of claim 27, wherein increasing stress tolerance comprises one or more of: decreasing programmed cell death, decreasing effector-triggered immunity (ETI)-induced cell death, increasing formation of NPR1 condensates, and degrading EDS1 and specific WRKY transcription factors required for pathogen ETI.

33. (canceled)

34. The method of claim 27, wherein the plant is a food crop plant, a biofuel plant, a corn plant, a legume plant, a bean plant, a rice plant, a soybean plant, a cotton plant, a sugarcane plant, a tobacco plant, a palm oil plant, a date palm, a wheat, a vegetable plant, a squash plant, a Solanaceae plant, a tomato, a banana plant, a potato plant, a pepper plant, a moss plant, a parsley plant, a sunflower plant, a mustard plant, a sorghum plant, a millet plant, a citrus plant, an apple plant, a strawberry plant, a rapeseed plant, a cabbage plant, a cassava plant, a coffee plant, a sweet potato plant, a jatropha plant, or a switchgrass plant.

35. (canceled)

36. (canceled)


SEQUENCE LISTING (168 sequences)

SEQ ID NO: 1 - protein, 593 aa, Arabidopsis thaliana
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 2 - protein, 588 aa, Nicotiana benthamiana
MDNSGTAFSDSNDISGSSSICCIGGGMTESFSPETSPAEITSLKRLSETLESIFDAASPEFDYFADAKLVIPGAGKEIPVHRCILSARSPFFKNLFCGKKEKNSNKVELKEIMKEYEVSYDGVVSVLAYLYSGKIRPSPKDVCVCVDNDCSHVACRPAVAFLAEVLYTSFTFQISELVDKFQRHLLDILDKIAADDVMVVLSVANICGKACERLLSSCIEIIVKSNVDIITLDKALPHDIVKQITDSRAELGLQGPESNGFPDKHVKRIHRALDSDDVELLQMLLREGHTTLDDAFALHYAVAYCDAKTTAELLDLALAEINHQNSRGYTVLHVAAMRKEPKIIVSLLTKGARPSDLTSDGRKALQIAKRLTRLVDFSKSPEEGKSASKDRLCIEILEQAERRDPLLGEASVSLAMAGDDLRMKLLYLENRVGLAKLLFPMEAKVAMDIAQVDGTSEFPLASINKKMVNAQRTTVDLNEVPFKIKEEHLNRLRALSRTVELGKRFFPRCSEVLNKIMDADDLSEIAYMGNDTAEERQLKKQRYMELQEILSKAFTEDKEEFDMTNNISSSCSSTSKGVDKPNKLPFRK

SEQ ID NO: 3 - protein, 576 aa, Solanum lycopersicon
MDSRTAFSDSNDISGSSSICCMNESETSLADVNSLKRLSETLESIFDASAPDFDFFADAKLLAPGGKEIPVHRCILSARSPFFKNVFCGKDSNTKLELKELMKEYEVSFDAVVSVLAYLYSGKVRPASKDVCVCVDNECLHVACRPAVAFMVQVLYASFTFQISQLVDKFQRHLLDILDKAVADDVMMVLSVANICGKACERLLSRCIDIIVKSNVDIITLDKSLPHDIVKQITDSRAELGLQGPESNGFPDKHVKRIHRALDSDDVELLRMLLKEGHTTLDDAYALHYAVAYCDAKTTAELLDLSLADVNHQNPRGHTVLHVAAMRKEPKIIVSLLTKGARPSDLTSDGKKALQIAKRLTRLVDFTKSTEEGKSAPKDRLCIEILEQAERRDPLLGEASLSLAMAGDDLRMKLLYLENRVGLAKLLFPMEAKVAMDIAQVDGTSELPLASMRKKIADAQRTTVDLNEAPFKMKEEHLNRLRALSRTVELGKRFFPRCSEVLNKIMDADDLSEIAYMGNDTVEERQLKKQRYMELQEILSKAFTEDKEEFAKTNMSSSCSSTSKGVDKPNNLPFRK

SEQ ID NO: 4 - protein, 584 aa, Vitis vinifera
MDYRAALSDSNDFSGSSSICCIAATTESLSSEVSPPDISALRRLSENLESVFESPEFDFFTDARIVVAGGREVPVHRCILAARSVFFKAVLAGARKEKEAKFELKDLAKEFDVGYDSLVAVLGYLYSGRVGALPKGVCACVDDDCPHSACRPAVDFMVEVLYASFAFQISELVGLYQRRLMDILDKVASDDILVILSVANLCGKACDRLLARCIDIIIKSDVDVVTLERALPQEMVKQIVDSRLELGFEEPESTNFPDKHVKRIHRALDSDDVELVRMLLKEGHTTLDDAYALHYAVAFGDAKTTTELLDLGLADVNHKNHRGYTVLHIAAMRKEPKIIVSLLTKGARPTDITPDGRNALQIAKRLTRAVDYHKSTEEGKPSPKDQLCVEVLEQAERRDPLIGEASFSLAIAGDDLRMKLLYLENRVGLAKLLFPMEAKVAMDIAQVDGTSEFTLTAIRPRNLADAQRTTVDLNEAPFRIKEEHLNRLRALSKTVDLGKRFFPRCSEVLNKIMDADDLSDLAYLGNGTTEERLLKKRRYKELQDQLCKAFNEDKEENDKSRISSSSSSTSLGFGRNNSRLSCKK

SEQ ID NO: 5 - protein, 576 aa, Hordeum vulgare
MEAPSSHVTTSFSDCDSVSMEDAAPDADVEALRRLSDNLAAAFRSPDDFAFLADARFAVPGAPDLCVHRCVLSARSPFLRALFKRRAAAAGSAGGAEGDRVELRELLGGEVEVGYEALRLVLDYLYSGRVCDLPKTACACVDEGGCAHVGCHPAVSFMAQVLFAASTFQVGELASLFQRHLLDLLDKVEADNLPLVLSVANLCNKSCVKLFERCLERVVRSDLDMITLDKALPLDVIKQIIDSRITLGLASPEDNGFPNKHVRRILSALDSDDVELVRLLLKEGQTNLDDAFALHYAVEHCDSKITTELLDIALADVNLRNPRGYTVLHIAARRRDPKIVVSLLTKGARPSDFTFDGRKAVQIAKRLTKHGDYFGNTEEGKPSPNDKLCIEILEEAERRDPQLGEASVSLALAGDCLRGKLLYLENRVALARIMFPIEARVAMDIAQVDGTLEFTLGSCTNPPPEITTVDLNDTPFKMKDEHLARMRALSKTVELGKRFFPRCSNVLDKIMDDEPELASLGRDASSERKRRFHDLHDTLLKAFSEDKEEFARSATLSASSSSTPTVARNLTGRPRR

SEQ ID NO: 6 - protein, 584 aa, Medicago truncatula
MMYLRSGFSEYSNEISNNTSSELCCTTAPNSTITASQDVITFTQPLNRLSDNLASILDDTGFDFFSDAKIIAKDGREVSVHRCILSARSSFFKDVFKGKKETTLQLKEVAKDYDVGFDALNVVLRYLYSERVEDHHLSAKDVCVCVDDDCLHFGCWPVVDFMLQLLYASFTFQISELLALYQDHLLDILDKMAIDDMLVVLSIANICGKTCDKLLKRCTDIIVESNVDITTLEKSLPQSIVKLVTYKRKQLGLDMYETVNLLDKHVTRIHRALDSDDVELVRLLLKEGHTTLDEAHALHYAVAYCDVKTTTELLDLGLADVNHKNLRGYSVLHVAAKRKEPKIIVSLLTKGAQPSELTMDGRKALQISKRCTKAVDYYKSTEEGKVSSNDRLCIEILEQAERREPLHGEASLSLAKAGDDLRMKLLYLENRVGLAKLLFPMEAKVVMDITPIDGTSEFTPNLGGYQRTTMDLNEAPFKIKEEHLIRMKALSRAVELGKRFFPRCSEVLNKIMDADDLSQLACMGHDSPEDRQVKRRRYAELQEVLNKVFHEDKEEFDKSGMSSSSSSTSIGMPRANNSMIAMNH

SEQ ID NO: 7 - protein, 588 aa, Nicotiana tabacum
MDNSRTAFSDSNDISGSSSICCIGGGMTEFFSPETSPAEITSLKRLSETLESIFDASLPEFDYFADAKLVVSGPCKEIPVHRCILSARSPFFKNLFCGKKEKNSSKVELKEVMKEHEVSYDAVMSVLAYLYSGKVRPSPKDVCVCVDNDCSHVACRPAVAFLVEVLYTSFTFQISELVDKFQRHLLDILDKTAADDVMMVLSVANICGKACERLLSSCIEIIVKSNVDIITLDKALPHDIVKQITDSRAELGLQGPESNGFPDKHVKRIHRALDSDDVELLQMLLREGHTTLDDAYALHYAVAYCDAKTTAELLDLALADINHQNSRGYTVLHVAAMRKEPKIVVSLLTKGARPSDLTSDGRKALQIAKRLTRLVDFSKSPEEGKSASNDRLCIEILEQAERRDPLLGEASVSLAMAGDDLRMKLLYLENRVGLAKLLFPMEAKVAMDIAQVDGTSEFPLASIGKKMANAQRTTVDLNEAPFKIKEEHLNRLRALSRTVELGKRFFPRCSEVLNKIMDADDLSEIAYMGNDTAEERQLKKQRYMELQEILTKAFTEDKEEYDKTNNISSSCSSTSKGVDKPNKLPFRK

SEQ ID NO: 8 - protein, 582 aa, Oryza sativa
MEPPTSHVTNAFSDSDSASVEEGGADADADVEALRRLSDNLAAAFRSPEDFAFLADARIAVPGGGGGGGDLLVHRCVLSARSPFLRGVFARRAAAAAGGGGEDGGERLELRELLGGGGEEVEVGYEALRLVLDYLYSGRVGDLPKAACLCVDEDCAHVGCHPAVAFMAQVLFAASTFQVAELTNLFQRRLLDVLDKVEVDNLLLILSVANLCNKSCMKLLERCLDMVVRSNLDMITLEKSLPPDVIKQIIDARLSLGLISPENKGFPNKHVRRIHRALDSDDVELVRMLLTEGQTNLDDAFALHYAVEHCDSKITTELLDLALADVNHRNPRGYTVLHIAARRREPKIIVSLLTKGARPADVTFDGRKAVQISKRLTKQGDYFGVTEEGKPSPKDRLCIEILEQAERRDPQLGEASVSLAMAGESLRGRLLYLENRVALARIMFPMEARVAMDIAQVDGTLEFNLGSGANPPPERQRTTVDLNESPFIMKEEHLARMTALSKTVELGKRFFPRCSNVLDKIMDDETDPVSLGRDTSAEKRKRFHDLQDVLQKAFHEDKEENDRSGLSSSSSSTSIGAIRPRR

SEQ ID NO: 9 - protein, 574 aa, Glycine max
  misc_feature (166)..(166): Xaa can be any naturally occurring amino acid
MNFRSGSSDSKDASNSSTGEAYLSGVSDVITPLRRLSEQLGSILDGGGVDFFSDAKIVAGDGREVAVNRCILAARSGFFKHVFAGGGGCVLRLKEVAKDYNVGLEALGIVLAYLYSGRVKPLPQGGVCVCVDDVCSHFGCRPAIDFLLQLLYASSTFQLNELIALXQGHLLDILEKVAIDDILVVLSVANICGIVCERLLARCTEMILKSDADITTLEKALPQHLVKQITDKRIELDLYMPENFNFPDKHVNRIHRALDSDDVELVRLLLKEGHTTLDDAYALHYAVAYCDVKTTTELLDLGLADVNHKNYRGYSVLHVAAMRKEPKIIVSLLTKGAQPSDLTLDGRKALQISKRLTKAVDYYKSTEEGKVSCSDRLCIEILEQAERREPLLGEASLSLAMAGDDLRMKLLYLENRVGLAKVLFPMEAKVIMDISQIDGTSEFPSTDMYCPNISDHQRTTVDLNDAPFRMKEEHLVRLRALSRTVELGKRFFPRCSEVLNKIMDADDLTQLTCMGDDSPEDRLRKRRRYVELQEVLNKVFNEDKEEFDRSAMSSSSSSTSIGVVRPNANLAMKN

SEQ ID NO: 10 - protein, 589 aa, Populus trichocarpa
MDNRIGFSDSNEISNGSSTCCIETPSTSKPFTNPEIVALQQLSGNLEAIFDSQDFDYFADAKITSSNYNREVPVHRCILSARSPFFKSVFSSPVAKDRSGVAKFELKELAKDYDVGFDSLMTVLGYLYCGKVRPWPKDVCACVDDDCSHIACRPAVDLLTEVLYASFTFQVNELVALYQRHLLDILDKVSTDDILVILAVANICGEACERLLTRCVEIIVKSNVDIVTLDKALPQYIVKKIMDSRLELGLNVPENSNLLDKHVKRIHRALDSDDVELVRMLLKEAHTNLDDAHALHYAVSYCDAKTTTEILDLGLADVNCRNSRGYTVLHVAAMRKDPKIIVSLLTKGARLSDLTLDGRKALQISKRLTRAMDYHKSTEEGKASPKERLCIEILEQAERRDPLLGEASLSLAMAGDDLRMKLLYLENRVGLAKLLFPMEAKVAMDIAQVDGTSEFPLAGIRPSILSGAQRGAVDLNEAPFRMHEEHLNRMRALSRTVELGKRFFPRCSDVLNKIMDADDLSQIAYLGNETSEERLVKRQRHLELQDALSKAFNEDKQEFDRSVISSSSSTKSIGTARSNGKLIDMGGGH

SEQ ID NO: 11 - protein, 586 aa, Cucumis melo
MADSHEPSSSLSFTSSSYTNGSQSCNMSPSSISDPMPSLEVISLNKLSSNLAQLLIHDGCDYTDADIVVEGVPVGIHRCILAVRSRFFHYLFQKDEKPAMKDGKPQYHMNELLPYGKVGHEAFLILLSYLYSGKLNSSPANVSTCVDNSCAHDACGPAIDFAVQLTYASSIFQIPELVSLFQRHLLNYVVKALVENVIQILVVAFHCQLSPLVTQCIDRIARSDLDCASLEKGLPYEVAERIKLVRLKSRGGDEQNLVADSPRDKKIKKICLALDSDDVELMKLLLSESDVTLDEANALHYAAAYCDPKSLTEVLNLNIADVNLRNSRGYTVLHVAAMRKDPSVIMSLLNKGAWAFDLTPDGRTAANICQRLTRPKDYHAKTEKGQETNKDRLCIDILEREMWRNPTSDSSILSLAMADDVHMKLIYLENRVAFARLLFPSEARLAMDIANADTTSEFVGLSMPKNSNKNLREVNLNETPSVQNKRFLSRMQALLKTVEMGRRFFPNCSEALDKFVADDLPDLFYLEKGTVEEQRIKRKRFKELKNDVQKAFDKDKAAKLNQSGLSPSSSSTSLKHGTNHRNVRRQ

SEQ ID NO: 12 - protein, 583 aa, Zea mays
MEPMDSQLTALALSDSDSASVEGAAADAADADLQALRRLSDNLAAAFRSPDDFAFLADARIVVPGAPDLRVHRCVLCARSPFLRDAFARRAASAGEEEKDKDSYMCKVELRDLLGDEVEVGYDALRLVLDYLYSGRVAALPKAACLCVDEDACAHVGCRPAVAFMAQVLFAASTFDVAELTNLFQRRLLDVLDKVEVDNLPLVLSVANLCSKSCVKLLERCLDVVVRSNLDMIALEKKLPPDVVKEIVDARVSLGLVSPEDKGFPNIHVRRIHRALDSDDVELVRMLLKEGKTNLDDAYALHYAVEHCDSKITTELLDLALADVNHRNPRGYTVLHIAAMRREPKIIVSLLTKGARPSDLTFDDRKAVQISKRLTKHGDYFGPTEDGKPSPKDRLCIEVLEQAERRDPQLGEASVSLAIEGDSARGRLLYLENRVALARILFPMEARVAMDIAQVDGTLEFTLVSSVNLPAEIQRTVDLNDTPFTMKEEHLARMRALSKTVEVGKRFFPRCSKVLDTIMDDEAEMASLGRDTSAEKKRRFHDLQDLVQKAFSEDKEENDRSAARSPSSSSTTTTSIGAVRPRR

SEQ ID NO: 13 - protein, 41 aa, Arabidopsis thaliana
RTALMIAKQATMAVECNNIPEQCKHSLKGRLCVEILEQEDK

SEQ ID NO: 14 - protein, 41 aa, Nicotiana benthamiana
RKALQIAKRLTRLVDFSKSPEEGKSASKDRLCIEILEQAER

SEQ ID NO: 15 - protein, 41 aa, Solanum Lycopersicon
KKALQIAKRLTRLVDFTKSTEEGKSAPKDRLCIEILEQAER

SEQ ID NO: 16 - protein, 41 aa, Vitis vinifera
RNALQIAKRLTRAVDYHKSTEEGKPSPKDQLCVEVLEQAER

SEQ ID NO: 17 - protein, 41 aa, Hordeum vulgare
RKAVQIAKRLTKHGDYFGNTEEGKPSPNDKLCIEILEEAER

SEQ ID NO: 18 - protein, 41 aa, Medicago truncatula
RKALQISKRCTKAVDYYKSTEEGKVSSNDRLCIEILEQAER

SEQ ID NO: 19 - protein, 41 aa, Nicotiana tabacum
RKALQIAKRLTRLVDFSKSPEEGKSASNDRLCIEILEQAER

SEQ ID NO: 20 - protein, 41 aa, Oryza sativa
RKAVQISKRLTKQGDYFGVTEEGKPSPKDRLCIEILEQAER

SEQ ID NO: 21 - protein, 41 aa, Glycine max
RKALQISKRLTKAVDYYKSTEEGKVSCSDRLCIEILEQAER

SEQ ID NO: 22 - protein, 41 aa, Populus trichocarpa
RKALQISKRLTRAMDYHKSTEEGKASPKERLCIEILEQAER

SEQ ID NO: 23 - protein, 41 aa, Cucumis melo
RTAANICQRLTRPKDYHAKTEKGQETNKDRLCIDILEREMW

SEQ ID NO: 24 - protein, 41 aa, Zea mays
RKAVQISKRLTKHGDYFGPTEDGKPSPKDRLCIEVLEQAER

SEQ ID NO: 25 - protein, 41 aa, Raphanus sativus
RTALVIAKQVTKATECCILERGKLSAKGGVCVEILKEPDNK

SEQ ID NO: 26 - protein, 41 aa, Brassica napus
RTALLIAKQVTKAAECCILEKGKLAAKGGVCVEILKQPDNK

SEQ ID NO: 27 - protein, 41 aa, Brassica oleracea
RTALLIAKQVTKAAECCILEKGKLAAKGGVCVEILKQPDNT

SEQ ID NO: 28 - protein, 41 aa, Brassica rapa; identical to SEQ ID NO: 26

SEQ ID NO: 29 - protein, 41 aa, Brassica juncea; identical to SEQ ID NO: 26

SEQ ID NO: 30 - protein, 608 aa, Brassica napus
METIARFDDFYEISSTSFPAAPAPTNNSGSSTVFPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADESCCHVACRPAVDFMVEVLYLAFVFQIQELVTMYQRHLLDVVDKVMIEDTLVVLKLANICGKACKKLFDKCREIIVKSNVDVVTLKKSLPENIAKQVIDIRKELGLEVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVCVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGTDSRGFFGIIFYMLQPSVHHLKKVSRVPPRTTTSVPSNPEGL

SEQ ID NO: 31 - protein, 579 aa, Brassica rapa
METIAGFDDFYEISSTSFLAAPAPTDNSGSSTVYPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVNIEDTLVVLKLANICGKACKKLFDKCREIIVKSNVDVVTLKKSLPENIAKQVIDIRKELGLEVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVCVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 32 - protein, 606 aa, Brassica oleracea
METIAGFDDFYEISSTSFLAAPAPTDNSGSSTVYPTELFTRPEVSAFQLLSNSLESVFDSPEAFYSDAKLVLSDDKEVSFHRCILSARSLFFKAALTAAEKVQKSTPVKLELKTLAAEYDVGFDSVVAVLAYVYSGRVRPPPKGVSECADESCCHVACRPAVDFMVEVLYLAFVFQIQELVTMYQRHLLDVVDKVMIEDTLVVLKLANICGKACKKLFDKCREIIVKSNVDVVTLKKSLPEDIAKQVIDIRKELGLEVAEPEKHVSNIHKALESDDLDLVVMLLKEGHTNLDEAYALHFAVAYCDEKTARNLLELGFADVNRRNPRGYTVIHVAAMRKEPTLIALLLTKGANALEMSLDGRTALLIAKQVTKAAECCILEKGKLAAKGGVCVEILKQPDNTREPFPEDVSPSLAVAADQFKIRLIDLENRVQMARCLYPMEAQVAMDFARMKGTREFVVTTATDLHMEPFKFVEMHQSRLTALSKTGIIKSFVTKITETNLCLTDLVFLLFLPVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 33 - protein, 581 aa, Raphanus sativus
METIAGFDDFYEISSTSFLAAPAPTDNSGSSTVYPTELLTTRPEVSASQLLSNSLESVFDSPEEFYSDAKLVLSDDREVSFHRCILSARSPFFKAELAAAEKVQKSTPVKLELKKLAAEYDVGFDSVVAVLAYVYCGRVRPPPKGVSECADESCCHVACRPAVDFMVEVLYLAFVFQIPELVTMYQRHLLDVIEKVIIEDTLVVLKLANICGKACKKLFDKCKEIIVMSDVDVVTLKKSLPEDVAKQVIDIRKELGLEVAEPEKHVSNIHKALESDDLALVDMLLNEGHTNLDDAYALHFAVAYCDVQTAKDLLELELADVNRRNPRGYTVLHVAAMRKEPTLIALLLTKGANASETSLDGRTALVIAKQVTKATECCILERGKLSAKGGVCVEILKEPDNKREPFPEDVSPSLAVAADELKIRLIDLENRVQMARCLYPMEAQVAMDFARMKGTHEFVVTTTTDLNMEPFKFVEMHRSRLTALSKTVEFGKRFFPRCSKVLDDILNFEDLTILALVEEETPEQRQQKRQRFMEIQEIVRMAFSKDKEDLGKSSLSPSSSSTSKLNGKKRSIAKLSHRRRR

SEQ ID NO: 34 - protein, 579 aa, Brassica juncea
METIARFDDFYEISSTSFPAAPAPTDNSGSSTVYPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVNIEDTLVVLKLANICGKACKKLFDKCREIIVKSNVDVVTLKKSLPENIAKQVIDIRKELGLDVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVCVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 35 - DNA, 55 bp, Artificial Sequence
  synthetic oligonucleotide
ggggacaagtttgtacaaaaaagcaggcttaatggacaccaccattgatggattc

SEQ ID NO: 36 - DNA, 58 bp, Artificial Sequence
  synthetic oligonucleotide
ggggacaagtttgtacaaaaaagcaggcttaatgatatgtggtaaagcttgtatgaag

SEQ ID NO: 37 - DNA, 56 bp, Artificial Sequence
  synthetic oligonucleotide
ggggaccactttgtacaagaaagctgggtcattagcaagcttgagtataaccaatg

SEQ ID NO: 38 - DNA, 57 bp, Artificial Sequence
  synthetic oligonucleotide
ggggacaagtttgtacaaaaaagcaggcttaatgtctaatgtagatatggttagtct

SEQ ID NO: 39 - DNA, 53 bp, Artificial Sequence
  synthetic oligonucleotide
ggggaccactttgtacaagaaagctgggtccaaagttgcttctgatgcacttg

SEQ ID NO: 40 - DNA, 59 bp, Artificial Sequence
  synthetic oligonucleotide
ggggacaagtttgtacaaaaaagcaggcttagagccacaattgatactatctctattgg

SEQ ID NO: 41 - DNA, 48 bp, Artificial Sequence
  synthetic oligonucleotide
ggggaccactttgtacaagaaagctgggtcccgacgacgatgagagag

SEQ ID NO: 42 - DNA, 40 bp, Artificial Sequence
  synthetic oligonucleotide
catacatatgtcccctatactaggttattggaaaattaag

SEQ ID NO: 43 - DNA, 36 bp, Artificial Sequence
  synthetic oligonucleotide
catacctaggtcattttggaggatggtcgccaccac

SEQ ID NO: 44 - DNA, 39 bp, Artificial Sequence
  synthetic oligonucleotide
catacctaggtcaattagcaagcttgagtataaccaatg

SEQ ID NO: 45 - DNA, 36 bp, Artificial Sequence
  synthetic oligonucleotide
gacgctgctcgatcttgaaaataaagttgcacttgc

SEQ ID NO: 46 - DNA, 36 bp, Artificial Sequence
  synthetic oligonucleotide
gcaagtgcaactttattttcaagatcgagcagcgtc

SEQ ID NO: 47 - DNA, 37 bp, Artificial Sequence
  synthetic oligonucleotide
gccgcctaaaggagtttctgaagctgcagacgagaat

SEQ ID NO: 48 - DNA, 37 bp, Artificial Sequence
  synthetic oligonucleotide
attctcgtctgcagcttcagaaactcctttaggcggc

SEQ ID NO: 49 - DNA, 59 bp, Artificial Sequence
  synthetic oligonucleotide
gtttctgaagctgcagacgagaatgcggctcacgtggctgctcggccggcggtggattt

SEQ ID NO: 50 - DNA, 59 bp, Artificial Sequence
  synthetic oligonucleotide
aaatccaccgccggccgagcagccacgtgagccgcattctcgtctgcagcttcagaaac

SEQ ID NO: 51 - DNA, 39 bp, Artificial Sequence
  synthetic oligonucleotide
gccactatggcggttgaagcgaataatatcccggagcaa

SEQ ID NO: 52 - DNA, 39 bp, Artificial Sequence
  synthetic oligonucleotide
ttgctccgggatattattcgcttcaaccgccatagtggc

SEQ ID NO: 53 - DNA, 41 bp, Artificial Sequence
  synthetic oligonucleotide
aataatatcccggagcaagctaagcattctctcaaaggccg

SEQ ID NO: 54 - DNA, 41 bp, Artificial Sequence
  synthetic oligonucleotide
cggcctttgagagaatgcttagcttgctccgggatattatt

SEQ ID NO: 55 - DNA, 49 bp, Artificial Sequence
  synthetic oligonucleotide
aagcattctctcaaaggccgactagcagtagaaatactagagcaagaag

SEQ ID NO: 56 - DNA, 49 bp, Artificial Sequence
  synthetic oligonucleotide
cttcttgctctagtatttctactgctagtcggcctttgagagaatgctt

SEQ ID NO: 57 - DNA, 48 bp, Artificial Sequence
  synthetic oligonucleotide
cgggaaacgattcttcccgcgcgcatcggcagtgctcgaccagattat

SEQ ID NO: 58 - DNA, 48 bp, Artificial Sequence
  synthetic oligonucleotide
ataatctggtcgagcactgccgatgcgcgcgggaagaatcgtttcccg

SEQ ID NO: 59 - DNA, 44 bp, Artificial Sequence
  synthetic oligonucleotide
tgctcgaccagattatgaacgcagaggacttgactcaactggct

SEQ ID NO: 60 - DNA, 44 bp, Artificial Sequence
  synthetic oligonucleotide
agccagttgagtcaagtcctctgcgttcataatctggtcgagca

SEQ ID NO: 61 - DNA, 43 bp, Artificial Sequence
  synthetic oligonucleotide
gaggacttgactcaactggctgcaggagaagacgacactgctg

SEQ ID NO: 62 - DNA, 43 bp, Artificial Sequence
  synthetic oligonucleotide
cagcagtgtcgtcttctcctgcagccagttgagtcaagtcctc

SEQ ID NO: 63 - DNA, 59 bp, Artificial Sequence
  synthetic oligonucleotide
ggggacaagtttgtacaaaaaagcaggcttaatgagtaatcagaagaagaggaattttc

SEQ ID NO: 64 - DNA, 56 bp, Artificial Sequence
  synthetic oligonucleotide
ggggaccactttgtacaagaaagctgggtattaggctagatagcggtaaagtttcc

SEQ ID NO: 65 - DNA, 56 bp, Artificial Sequence
  synthetic oligonucleotide
ggggacaagtttgtacaaaaaagcaggcttaagcaatcagaagaagcgtaactttc

SEQ ID NO: 66 - DNA, 56 bp, Artificial Sequence
  synthetic oligonucleotide
ggggaccactttgtacaagaaagctgggtattacgccagataacgatacagtttgc

SEQ ID NO: 67 - DNA, 48 bp, Artificial Sequence
  synthetic oligonucleotide
gaaacatgaactgaacgtgtcgactatcgaacaggctacagaaatccc

SEQ ID NO: 68 - DNA, 48 bp, Artificial Sequence
  synthetic oligonucleotide
gggatttctgtagcctgttcgatagtcgacacgttcagttcatgtttc

SEQ ID NO: 69 - DNA, 35 bp, Artificial Sequence
  synthetic oligonucleotide
catacatatggaacagaaactgatctctgaagaag

SEQ ID NO: 70 - DNA, 36 bp, Artificial Sequence
  synthetic oligonucleotide
catactcgagttacgccagataacgatacagtttgc

SEQ ID NO: 71 - DNA, 31 bp, Artificial Sequence
  synthetic oligonucleotide
cataccatggtggcgaccctggacagcgatg

SEQ ID NO: 72 - DNA, 40 bp, Artificial Sequence
  synthetic oligonucleotide
cataaagcttttagtgaccatacttctgaaactcccattc

SEQ ID NO: 73 - DNA, 57 bp, Artificial Sequence
  synthetic oligonucleotide
ggggacaagtttgtacaaaaaagcaggcttaatgcaaatcttcgtgaaaacactcac

SEQ ID NO: 74 - DNA, 54 bp, Artificial Sequence
  synthetic oligonucleotide
ggggaccactttgtacaagaaagctgggtattaaccacctcttaaacggagaac

SEQ ID NO: 75 - DNA, 37 bp, Artificial Sequence
  synthetic oligonucleotide
cataccatggtgtacccatacgatgttccagattacg

SEQ ID NO: 76 - DNA, 34 bp, Artificial Sequence
  synthetic oligonucleotide
catagaattcttaaccacctcttaaacggagaac

SEQ ID NO: 77 - DNA, 36 bp, Artificial Sequence
  synthetic oligonucleotide
cataccatggtggcttcgaaacggatcttgaaggag

SEQ ID NO: 78 - DNA, 51 bp, Artificial Sequence
  synthetic oligonucleotide
cataaagcttttagcccattgcatacttctgagtccagtttcttgcagtag

SEQ ID NO: 79 - DNA, 41 bp, Artificial Sequence
  synthetic oligonucleotide
catagaattcgaaattaatacgactcactataggggaattg

SEQ ID NO: 80 - DNA, 20 bp, Artificial Sequence
  synthetic oligonucleotide
gattatgcggccgtgtacaa

SEQ ID NO: 81 - DNA, 37 bp, Artificial Sequence
  synthetic oligonucleotide
cataggccggccaccttcacaagcgagctagtgaagc

SEQ ID NO: 82 - DNA, 37 bp, Artificial Sequence
  synthetic oligonucleotide
catacctaggtcacctgaagtagatagagacgagagg

SEQ ID NO: 83 - DNA, 56 bp, Artificial Sequence
  synthetic oligonucleotide
ggggacaagtttgtacaaaaaagcaggcttaatggatactaataaagcaaaaaagc

SEQ ID NO: 84 - DNA, 55 bp, Artificial Sequence
  synthetic oligonucleotide
ggggaccactttgtacaagaaagctgggtaagatagattcgaacatgaactgaag

SEQ ID NO: 85 - DNA, 36 bp, Artificial Sequence
  synthetic oligonucleotide
cataccatggtggactacaaagacgatgacgacaaa

SEQ ID NO: 86 - DNA, 40 bp, Artificial Sequence
  synthetic oligonucleotide
catagcggccgcttaagatagattcgaacatgaactgaag

SEQ ID NO: 87 - DNA, 60 bp, Artificial Sequence
  synthetic oligonucleotide
ggggacaagtttgtacaaaaaagcaggcttaatggattcgaatagtaacaacacgaaatc

SEQ ID NO: 88 - DNA, 57 bp, Artificial Sequence
  synthetic oligonucleotide
ggggaccactttgtacaagaaagctgggtacatagcacttgttctttcataatcagc

SEQ ID NO: 89 - DNA, 55 bp, Artificial Sequence
  synthetic oligonucleotide
ggggacaagtttgtacaaaaaagcaggcttagaaggatccaagctacttccttgc

SEQ ID NO: 90 - DNA, 58 bp, Artificial Sequence
  synthetic oligonucleotide
ggggaccactttgtacaagaaagctgggtaatgttgttcaacctcctcatgtttggct

SEQ ID NO: 91 - DNA, 58 bp, Artificial Sequence
  synthetic oligonucleotide
ggggacaagtttgtacaaaaaagcaggcttagcgaacgaggtgattcttcttgatttc

SEQ ID NO: 92 - DNA, 57 bp, Artificial Sequence
  synthetic oligonucleotide
ggggaccactttgtacaagaaagctgggtactcaggtacaaatttcttcctgagctc

SEQ ID NO: 93 - DNA, 55 bp, Artificial Sequence
  synthetic oligonucleotide
ggggacaagtttgtacaaaaaagcaggcttaatggcgtttgaagctcttaccgga

SEQ ID NO: 94 - DNA, 58 bp, Artificial Sequence
  synthetic oligonucleotide
ggggaccactttgtacaagaaagctgggtaggtatctgttatttcatccatcatatag

SEQ ID NO: 95 - DNA, 60 bp, Artificial Sequence
  synthetic oligonucleotide
cataccatggtggactacaaagacgatgacgacaaagcgtttgaagctcttaccggaatc

SEQ ID NO: 96 - DNA, 43 bp, Artificial Sequence
  synthetic oligonucleotide
catagcggccgcttaggtatctgttatttcatccatcatatag

SEQ ID NO: 97 - DNA, 58 bp, Artificial Sequence
  synthetic oligonucleotide
ggggacaagtttgtacaaaaaagcaggcttaatgtatcctaaacaatttagtttatac

SEQ ID NO: 98 - DNA, 55 bp, Artificial Sequence
  synthetic oligonucleotide
ggggaccactttgtacaagaaagctgggtacaatgcaagattaagatctaaagcc

SEQ ID NO: 99 - DNA, 53 bp, Artificial Sequence
  synthetic oligonucleotide
ggggacaagtttgtacaaaaaagcaggcttagtaaattgtgtcctatacaaag

SEQ ID NO: 100 - DNA, 55 bp, Artificial Sequence
  synthetic oligonucleotide
ggggaccactttgtacaagaaagctgggttttttctaagttgataatggttattg

SEQ ID NO: 101 - DNA, 52 bp, Artificial Sequence
  synthetic oligonucleotide
ggggacaagtttgtacaaaaaagcaggcttaatggtgagcaagggcgaggag

SEQ ID NO: 102 - DNA, 71 bp, Artificial Sequence
  synthetic oligonucleotide
ttaatcttcgaccttgcgcttcttcttaggtggttctggtctagaggatcccttgtacagctcgtccatgc

SEQ ID NO: 103 - DNA, 52 bp, Artificial Sequence
  synthetic oligonucleotide
ggggaccactttgtacaagaaagctgggttttaatcttcgaccttgcgcttc

SEQ ID NO: 104 - DNA, 39 bp, Artificial Sequence
  synthetic oligonucleotide
cgacgacaagaccctgcaaacatttgtggtaaagcatgc

SEQ ID NO: 105 - DNA, 35 bp, Artificial Sequence
  synthetic oligonucleotide
gaggagaagagccctgctacagcataatggagagc

SEQ ID NO: 106 - DNA, 31 bp, Artificial Sequence
  synthetic oligonucleotide
gtgtcccactaacctcaatccccgttctccc

SEQ ID NO: 107 - DNA, 33 bp, Artificial Sequence
  synthetic oligonucleotide
ggattgaggttagtgggacactctttcaatctc

SEQ ID NO: 108 - DNA, 37 bp, Artificial Sequence
  synthetic oligonucleotide
cgacgacaagaccctagaccatatcatccactctagc

SEQ ID NO: 109 - DNA, 40 bp, Artificial Sequence
  synthetic oligonucleotide
gaggagaagagccctgtgcattgcatcctgagaagttttc

SEQ ID NO: 110 - DNA, 39 bp, Artificial Sequence; identical to SEQ ID NO: 104
  synthetic oligonucleotide

SEQ ID NO: 111 - DNA, 35 bp, Artificial Sequence; identical to SEQ ID NO: 105
  synthetic oligonucleotide

SEQ ID NO: 112 - DNA, 22 bp, Artificial Sequence
  synthetic oligonucleotide
gctttggtcttggcacctactc

SEQ ID NO: 113 - DNA, 20 bp, Artificial Sequence
  synthetic oligonucleotide
tgctcgcatgaccttttcaa

SEQ ID NO: 114 - DNA, 20 bp, Artificial Sequence
  synthetic oligonucleotide
gccaacacagtccgtcacta

SEQ ID NO: 115 - DNA, 18 bp, Artificial Sequence
  synthetic oligonucleotide
ttatcgcagacccccaaa

SEQ ID NO: 116 - DNA, 20 bp, Artificial Sequence
  synthetic oligonucleotide
tgtggatccgaaatatgctg

SEQ ID NO: 117 - DNA, 26 bp, Artificial Sequence
  synthetic oligonucleotide
aactaagaccactagcgttatgattg

SEQ ID NO: 118 - DNA, 22 bp, Artificial Sequence
  synthetic oligonucleotide
tgagtacgcttcttggactgat

SEQ ID NO: 119 - DNA, 20 bp, Artificial Sequence
  synthetic oligonucleotide
ttctcatcagtccccggtta

SEQ ID NO: 120 - DNA, 22 bp, Artificial Sequence
  synthetic oligonucleotide
ttcttggaagagctgaataggc

SEQ ID NO: 121 - DNA, 23 bp, Artificial Sequence
  synthetic oligonucleotide
tcagaatatcccgtatcatttgc

SEQ ID NO: 122 - DNA, 22 bp, Artificial Sequence
  synthetic oligonucleotide
tgtgtgtgtttgtgtggacaat

SEQ ID NO: 123 - DNA, 20 bp, Artificial Sequence
  synthetic oligonucleotide
gaacgctacagctggcctac

SEQ ID NO: 124 - DNA, 20 bp, Artificial Sequence
  synthetic oligonucleotide
cttcgaaggttcggttatgc

SEQ ID NO: 125 - DNA, 22 bp, Artificial Sequence
  synthetic oligonucleotide
gcttctcctagcagtggatctc

SEQ ID NO: 126 - DNA, 21 bp, Artificial Sequence
  synthetic oligonucleotide
gtgcccaaaattctcaacaag

SEQ ID NO: 127 - DNA, 23 bp, Artificial Sequence
  synthetic oligonucleotide
ttctacacctacatctgcacgag

SEQ ID NO: 128 - DNA, 20 bp, Artificial Sequence
  synthetic oligonucleotide
gatgatttctacagcgacgc

SEQ ID NO: 129 - DNA, 20 bp, Artificial Sequence
  synthetic oligonucleotide
ccatagcttaatgcagatgg

SEQ ID NO: 130 - DNA, 21 bp, Artificial Sequence
  synthetic oligonucleotide
ttcttgattcttttgatcccg

SEQ ID NO: 131 - DNA, 21 bp, Artificial Sequence
  synthetic oligonucleotide
gctggtgttgttctcttgctc

SEQ ID NO: 132 - DNA, 21 bp, Artificial Sequence
  synthetic oligonucleotide
tgatcttcggaatccatgaag

SEQ ID NO: 133 - DNA, 21 bp, Artificial Sequence
  synthetic oligonucleotide
caaaccacaccaagaggaaag

SEQ ID NO: 134 - protein, 593 aa, Artificial Sequence
  A. thaliana npr1 rdr2 protein
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVEANNIPEQAKHSLKGRLAVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 135 - protein, 593 aa, Artificial Sequence
  A. thaliana npr1 rdr1 protein
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSEAADENAAHVAARPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 136 - protein, 593 aa, Artificial Sequence
  A. thaliana npr1 rdr3 protein
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLCVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRASAVLDQIMNAEDLTQLAAGEDDTAEKRLQKKQRYMEIQETLKKAFSEDNLELGNSSLTDSTSSTSKSTGGKRSNRKLSHRRR

SEQ ID NO: 137 - protein, 588 aa, Artificial Sequence
  Nicotiana benthamiana npr1 rdr2 protein
MDNSGTAFSDSNDISGSSSICCIGGGMTESFSPETSPAEITSLKRLSETLESIFDAASPEFDYFADAKLVIPGAGKEIPVHRCILSARSPFFKNLFCGKKEKNSNKVELKEIMKEYEVSYDGVVSVLAYLYSGKIRPSPKDVCVCVDNDCSHVACRPAVAFLAEVLYTSFTFQISELVDKFQRHLLDILDKIAADDVMVVLSVANICGKACERLLSSCIEIIVKSNVDIITLDKALPHDIVKQITDSRAELGLQGPESNGFPDKHVKRIHRALDSDDVELLQMLLREGHTTLDDAFALHYAVAYCDAKTTAELLDLALAEINHQNSRGYTVLHVAAMRKEPKIIVSLLTKGARPSDLTSDGRKALQIAKRLTRLVDFSKSPEEGKSASKDRLAIEILEQAERRDPLLGEASVSLAMAGDDLRMKLLYLENRVGLAKLLFPMEAKVAMDIAQVDGTSEFPLASINKKMVNAQRTTVDLNEVPFKIKEEHLNRLRALSRTVELGKRFFPRCSEVLNKIMDADDLSEIAYMGNDTAEERQLKKQRYMELQEILSKAFTEDKEEFDMTNNISSSCSSTSKGVDKPNKLPFRK

SEQ ID NO: 138 - protein, 576 aa, Artificial Sequence
  Solanum lycopersicon npr1 rdr2 protein
MDSRTAFSDSNDISGSSSICCMNESETSLADVNSLKRLSETLESIFDASAPDFDFFADAKLLAPGGKEIPVHRCILSARSPFFKNVFCGKDSNTKLELKELMKEYEVSFDAVVSVLAYLYSGKVRPASKDVCVCVDNECLHVACRPAVAFMVQVLYASFTFQISQLVDKFQRHLLDILDKAVADDVMMVLSVANICGKACERLLSRCIDIIVKSNVDIITLDKSLPHDIVKQITDSRAELGLQGPESNGFPDKHVKRIHRALDSDDVELLRMLLKEGHTTLDDAYALHYAVAYCDAKTTAELLDLSLADVNHQNPRGHTVLHVAAMRKEPKIIVSLLTKGARPSDLTSDGKKALQIAKRLTRLVDFTKSTEEGKSAPKDRLAIEILEQAERRDPLLGEASLSLAMAGDDLRMKLLYLENRVGLAKLLFPMEAKVAMDIAQVDGTSELPLASMRKKIADAQRTTVDLNEAPFKMKEEHLNRLRALSRTVELGKRFFPRCSEVLNKIMDADDLSEIAYMGNDTVEERQLKKQRYMELQEILSKAFTEDKEEFAKTNMSSSCSSTSKGVDKPNNLPFRK

SEQ ID NO: 139 - protein, 584 aa, Artificial Sequence
  Vitis vinifera npr1 rdr2 protein
MDYRAALSDSNDFSGSSSICCIAATTESLSSEVSPPDISALRRLSENLESVFESPEFDFFTDARIVVAGGREVPVHRCILAARSVFFKAVLAGARKEKEAKFELKDLAKEFDVGYDSLVAVLGYLYSGRVGALPKGVCACVDDDCPHSACRPAVDFMVEVLYASFAFQISELVGLYQRRLMDILDKVASDDILVILSVANLCGKACDRLLARCIDIIIKSDVDVVTLERALPQEMVKQIVDSRLELGFEEPESTNFPDKHVKRIHRALDSDDVELVRMLLKEGHTTLDDAYALHYAVAFGDAKTTTELLDLGLADVNHKNHRGYTVLHIAAMRKEPKIIVSLLTKGARPTDITPDGRNALQIAKRLTRAVDYHKSTEEGKPSPKDQLAVEVLEQAERRDPLIGEASFSLAIAGDDLRMKLLYLENRVGLAKLLFPMEAKVAMDIAQVDGTSEFTLTAIRPRNLADAQRTTVDLNEAPFRIKEEHLNRLRALSKTVDLGKRFFPRCSEVLNKIMDADDLSDLAYLGNGTTEERLLKKRRYKELQDQLCKAFNEDKEENDKSRISSSSSSTSLGFGRNNSRLSCKK

SEQ ID NO: 140 - protein, 576 aa, Artificial Sequence
  Hordeum vulgare npr1 rdr2 protein
MEAPSSHVTTSFSDCDSVSMEDAAPDADVEALRRLSDNLAAAFRSPDDFAFLADARFAVPGAPDLCVHRCVLSARSPFLRALFKRRAAAAGSAGGAEGDRVELRELLGGEVEVGYEALRLVLDYLYSGRVCDLPKTACACVDEGGCAHVGCHPAVSFMAQVLFAASTFQVGELASLFQRHLLDLLDKVEADNLPLVLSVANLCNKSCVKLFERCLERVVRSDLDMITLDKALPLDVIKQIIDSRITLGLASPEDNGFPNKHVRRILSALDSDDVELVRLLLKEGQTNLDDAFALHYAVEHCDSKITTELLDIALADVNLRNPRGYTVLHIAARRRDPKIVVSLLTKGARPSDFTFDGRKAVQIAKRLTKHGDYFGNTEEGKPSPNDKLAIEILEEAERRDPQLGEASVSLALAGDCLRGKLLYLENRVALARIMFPIEARVAMDIAQVDGTLEFTLGSCTNPPPEITTVDLNDTPFKMKDEHLARMRALSKTVELGKRFFPRCSNVLDKIMDDEPELASLGRDASSERKRRFHDLHDTLLKAFSEDKEEFARSATLSASSSSTPTVARNLTGRPRR

SEQ ID NO: 141 - protein, 584 aa, Artificial Sequence
  Medicago truncatula npr1 rdr2 protein
MMYLRSGFSEYSNEISNNTSSELCCTTAPNSTITASQDVITFTQPLNRLSDNLASILDDTGFDFFSDAKIIAKDGREVSVHRCILSARSSFFKDVFKGKKETTLQLKEVAKDYDVGFDALNVVLRYLYSERVEDHHLSAKDVCVCVDDDCLHFGCWPVVDFMLQLLYASFTFQISELLALYQDHLLDILDKMAIDDMLVVLSIANICGKTCDKLLKRCTDIIVESNVDITTLEKSLPQSIVKLVTYKRKQLGLDMYETVNLLDKHVTRIHRALDSDDVELVRLLLKEGHTTLDEAHALHYAVAYCDVKTTTELLDLGLADVNHKNLRGYSVLHVAAKRKEPKIIVSLLTKGAQPSELTMDGRKALQISKRATKAVDYYKSTEEGKVSSNDRLAIEILEQAERREPLHGEASLSLAKAGDDLRMKLLYLENRVGLAKLLFPMEAKVVMDITPIDGTSEFTPNLGGYQRTTMDLNEAPFKIKEEHLIRMKALSRAVELGKRFFPRCSEVLNKIMDADDLSQLACMGHDSPEDRQVKRRRYAELQEVLNKVFHEDKEEFDKSGMSSSSSSTSIGMPRANNSMIAMNH

SEQ ID NO: 142 - protein, 588 aa, Artificial Sequence
  Nicotiana tabacum npr1 rdr2 protein
MDNSRTAFSDSNDISGSSSICCIGGGMTEFFSPETSPAEITSLKRLSETLESIFDASLPEFDYFADAKLVVSGPCKEIPVHRCILSARSPFFKNLFCGKKEKNSSKVELKEVMKEHEVSYDAVMSVLAYLYSGKVRPSPKDVCVCVDNDCSHVACRPAVAFLVEVLYTSFTFQISELVDKFQRHLLDILDKTAADDVMMVLSVANICGKACERLLSSCIEIIVKSNVDIITLDKALPHDIVKQITDSRAELGLQGPESNGFPDKHVKRIHRALDSDDVELLQMLLREGHTTLDDAYALHYAVAYCDAKTTAELLDLALADINHQNSRGYTVLHVAAMRKEPKIVVSLLTKGARPSDLTSDGRKALQIAKRLTRLVDFSKSPEEGKSASNDRLAIEILEQAERRDPLLGEASVSLAMAGDDLRMKLLYLENRVGLAKLLFPMEAKVAMDIAQVDGTSEFPLASIGKKMANAQRTTVDLNEAPFKIKEEHLNRLRALSRTVELGKRFFPRCSEVLNKIMDADDLSEIAYMGNDTAEERQLKKQRYMELQEILTKAFTEDKEEYDKTNNISSSCSSTSKGVDKPNKLPFRK

SEQ ID NO: 143 - protein, 582 aa, Artificial Sequence
  Oryza sativa npr1 rdr2 protein
MEPPTSHVTNAFSDSDSASVEEGGADADADVEALRRLSDNLAAAFRSPEDFAFLADARIAVPGGGGGGGDLLVHRCVLSARSPFLRGVFARRAAAAAGGGGEDGGERLELRELLGGGGEEVEVGYEALRLVLDYLYSGRVGDLPKAACLCVDEDCAHVGCHPAVAFMAQVLFAASTFQVAELTNLFQRRLLDVLDKVEVDNLLLILSVANLCNKSCMKLLERCLDMVVRSNLDMITLEKSLPPDVIKQIIDARLSLGLISPENKGFPNKHVRRIHRALDSDDVELVRMLLTEGQTNLDDAFALHYAVEHCDSKITTELLDLALADVNHRNPRGYTVLHIAARRREPKIIVSLLTKGARPADVTFDGRKAVQISKRLTKQGDYFGVTEEGKPSPKDRLAIEILEQAERRDPQLGEASVSLAMAGESLRGRLLYLENRVALARIMFPMEARVAMDIAQVDGTLEFNLGSGANPPPERQRTTVDLNESPFIMKEEHLARMTALSKTVELGKRFFPRCSNVLDKIMDDETDPVSLGRDTSAEKRKRFHDLQDVLQKAFHEDKEENDRSGLSSSSSSTSIGAIRPRR

SEQ ID NO: 144 - protein, 574 aa, Artificial Sequence
  Glycine max npr1 rdr2 protein
  misc_feature (166)..(166): Xaa can be any naturally occurring amino acid
MNFRSGSSDSKDASNSSTGEAYLSGVSDVITPLRRLSEQLGSILDGGGVDFFSDAKIVAGDGREVAVNRCILAARSGFFKHVFAGGGGCVLRLKEVAKDYNVGLEALGIVLAYLYSGRVKPLPQGGVCVCVDDVCSHFGCRPAIDFLLQLLYASSTFQLNELIALXQGHLLDILEKVAIDDILVVLSVANICGIVCERLLARCTEMILKSDADITTLEKALPQHLVKQITDKRIELDLYMPENFNFPDKHVNRIHRALDSDDVELVRLLLKEGHTTLDDAYALHYAVAYCDVKTTTELLDLGLADVNHKNYRGYSVLHVAAMRKEPKIIVSLLTKGAQPSDLTLDGRKALQISKRLTKAVDYYKSTEEGKVSCSDRLAIEILEQAERREPLLGEASLSLAMAGDDLRMKLLYLENRVGLAKVLFPMEAKVIMDISQIDGTSEFPSTDMYCPNISDHQRTTVDLNDAPFRMKEEHLVRLRALSRTVELGKRFFPRCSEVLNKIMDADDLTQLTCMGDDSPEDRLRKRRRYVELQEVLNKVFNEDKEEFDRSAMSSSSSSTSIGVVRPNANLAMKN

SEQ ID NO: 145 - protein, 589 aa, Artificial Sequence
  Populus trichocarpa npr1 rdr2 protein
MDNRIGFSDSNEISNGSSTCCIETPSTSKPFTNPEIVALQQLSGNLEAIFDSQDFDYFADAKITSSNYNREVPVHRCILSARSPFFKSVFSSPVAKDRSGVAKFELKELAKDYDVGFDSLMTVLGYLYCGKVRPWPKDVCACVDDDCSHIACRPAVDLLTEVLYASFTFQVNELVALYQRHLLDILDKVSTDDILVILAVANICGEACERLLTRCVEIIVKSNVDIVTLDKALPQYIVKKIMDSRLELGLNVPENSNLLDKHVKRIHRALDSDDVELVRMLLKEAHTNLDDAHALHYAVSYCDAKTTTEILDLGLADVNCRNSRGYTVLHVAAMRKDPKIIVSLLTKGARLSDLTLDGRKALQISKRLTRAMDYHKSTEEGKASPKERLAIEILEQAERRDPLLGEASLSLAMAGDDLRMKLLYLENRVGLAKLLFPMEAKVAMDIAQVDGTSEFPLAGIRPSILSGAQRGAVDLNEAPFRMHEEHLNRMRALSRTVELGKRFFPRCSDVLNKIMDADDLSQIAYLGNETSEERLVKRQRHLELQDALSKAFNEDKQEFDRSVISSSSSTKSIGTARSNGKLIDMGGGH

SEQ ID NO: 146 - protein, 586 aa, Artificial Sequence
  Cucumis melo npr1 rdr2 protein
MADSHEPSSSLSFTSSSYTNGSQSCNMSPSSISDPMPSLEVISLNKLSSNLAQLLIHDGCDYTDADIVVEGVPVGIHRCILAVRSRFFHYLFQKDEKPAMKDGKPQYHMNELLPYGKVGHEAFLILLSYLYSGKLNSSPANVSTCVDNSCAHDACGPAIDFAVQLTYASSIFQIPELVSLFQRHLLNYVVKALVENVIQILVVAFHCQLSPLVTQCIDRIARSDLDCASLEKGLPYEVAERIKLVRLKSRGGDEQNLVADSPRDKKIKKICLALDSDDVELMKLLLSESDVTLDEANALHYAAAYCDPKSLTEVLNLNIADVNLRNSRGYTVLHVAAMRKDPSVIMSLLNKGAWAFDLTPDGRTAANICQRLTRPKDYHAKTEKGQETNKDRLAIDILEREMWRNPTSDSSILSLAMADDVHMKLIYLENRVAFARLLFPSEARLAMDIANADTTSEFVGLSMPKNSNKNLREVNLNETPSVQNKRFLSRMQALLKTVEMGRRFFPNCSEALDKFVADDLPDLFYLEKGTVEEQRIKRKRFKELKNDVQKAFDKDKAAKLNQSGLSPSSSSTSLKHGTNHRNVRRQ

SEQ ID NO: 147 - protein, 583 aa, Artificial Sequence
  Zea mays npr1 rdr2 protein
MEPMDSQLTALALSDSDSASVEGAAADAADADLQALRRLSDNLAAAFRSPDDFAFLADARIVVPGAPDLRVHRCVLCARSPFLRDAFARRAASAGEEEKDKDSYMCKVELRDLLGDEVEVGYDALRLVLDYLYSGRVAALPKAACLCVDEDACAHVGCRPAVAFMAQVLFAASTFDVAELTNLFQRRLLDVLDKVEVDNLPLVLSVANLCSKSCVKLLERCLDVVVRSNLDMIALEKKLPPDVVKEIVDARVSLGLVSPEDKGFPNIHVRRIHRALDSDDVELVRMLLKEGKTNLDDAYALHYAVEHCDSKITTELLDLALADVNHRNPRGYTVLHIAAMRREPKIIVSLLTKGARPSDLTFDDRKAVQISKRLTKHGDYFGPTEDGKPSPKDRLAIEVLEQAERRDPQLGEASVSLAIEGDSARGRLLYLENRVALARILFPMEARVAMDIAQVDGTLEFTLVSSVNLPAEIQRTVDLNDTPFTMKEEHLARMRALSKTVEVGKRFFPRCSKVLDTIMDDEAEMASLGRDTSAEKKRRFHDLQDLVQKAFSEDKEENDRSAARSPSSSSTTTTSIGAVRPRR

SEQ ID NO: 148 - protein, 608 aa, Artificial Sequence
  Brassica napus npr1 rdr2 protein
METIARFDDFYEISSTSFPAAPAPTNNSGSSTVFPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADESCCHVACRPAVDFMVEVLYLAFVFQIQELVTMYQRHLLDVVDKVMIEDTLVVLKLANICGKACKKLFDKCREIIVKSNVDVVTLKKSLPENIAKQVIDIRKELGLEVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVAVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGTDSRGFFGIIFYMLQPSVHHLKKVSRVPPRTTTSVPSNPEGL

SEQ ID NO: 149 - protein, 579 aa, Artificial Sequence
  Brassica rapa npr1 rdr2 protein
METIAGFDDFYEISSTSFLAAPAPTDNSGSSTVYPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVNIEDTLVVLKLANICGKACKKLFDKCREIIVKSNVDVVTLKKSLPENIAKQVIDIRKELGLEVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVAVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 150 - protein, 606 aa, Artificial Sequence
  Brassica oleracea npr1 rdr2 protein
METIAGFDDFYEISSTSFLAAPAPTDNSGSSTVYPTELFTRPEVSAFQLLSNSLESVFDSPEAFYSDAKLVLSDDKEVSFHRCILSARSLFFKAALTAAEKVQKSTPVKLELKTLAAEYDVGFDSVVAVLAYVYSGRVRPPPKGVSECADESCCHVACRPAVDFMVEVLYLAFVFQIQELVTMYQRHLLDVVDKVMIEDTLVVLKLANICGKACKKLFDKCREIIVKSNVDVVTLKKSLPEDIAKQVIDIRKELGLEVAEPEKHVSNIHKALESDDLDLVVMLLKEGHTNLDEAYALHFAVAYCDEKTARNLLELGFADVNRRNPRGYTVIHVAAMRKEPTLIALLLTKGANALEMSLDGRTALLIAKQVTKAAECCILEKGKLAAKGGVAVEILKQPDNTREPFPEDVSPSLAVAADQFKIRLIDLENRVQMARCLYPMEAQVAMDFARMKGTREFVVTTATDLHMEPFKFVEMHQSRLTALSKTGIIKSFVTKITETNLCLTDLVFLLFLPVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 151 - protein, 581 aa, Artificial Sequence
  Raphanus sativus npr1 rdr2 protein
METIAGFDDFYEISSTSFLAAPAPTDNSGSSTVYPTELLTTRPEVSASQLLSNSLESVFDSPEEFYSDAKLVLSDDREVSFHRCILSARSPFFKAELAAAEKVQKSTPVKLELKKLAAEYDVGFDSVVAVLAYVYCGRVRPPPKGVSECADESCCHVACRPAVDFMVEVLYLAFVFQIPELVTMYQRHLLDVIEKVIIEDTLVVLKLANICGKACKKLFDKCKEIIVMSDVDVVTLKKSLPEDVAKQVIDIRKELGLEVAEPEKHVSNIHKALESDDLALVDMLLNEGHTNLDDAYALHFAVAYCDVQTAKDLLELELADVNRRNPRGYTVLHVAAMRKEPTLIALLLTKGANASETSLDGRTALVIAKQVTKATECCILERGKLSAKGGVAVEILKEPDNKREPFPEDVSPSLAVAADELKIRLIDLENRVQMARCLYPMEAQVAMDFARMKGTHEFVVTTTTDLNMEPFKFVEMHRSRLTALSKTVEFGKRFFPRCSKVLDDILNFEDLTILALVEEETPEQRQQKRQRFMEIQEIVRMAFSKDKEDLGKSSLSPSSSSTSKLNGKKRSIAKLSHRRRR

SEQ ID NO: 152 - protein, 579 aa, Artificial Sequence
  Brassica juncea npr1 rdr2 protein
METIARFDDFYEISSTSFPAAPAPTDNSGSSTVYPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVNIEDTLVVLKLANICGKACKKLFDKCREIIVKSNVDVVTLKKSLPENIAKQVIDIRKELGLDVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAECCILEKGKLAAKGGVAVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 153 - protein, 593 aa, Artificial Sequence
  A. thaliana npr1 rdr2 protein
MDTTIDGFADSYEISSTSFVATDNTDSSIVYLAAEQVLTGPDVSALQLLSNSFESVFDSPDDFYSDAKLVLSDGREVSFHRCVLSARSSFFKSALAAAKKEKDSNNTAAVKLELKEIAKDYEVGFDSVVTVLAYVYSSRVRPPPKGVSECADENCCHVACRPAVDFMLEVLYLAFIFKIPELITLYQRHLLDVVDKVVIEDTLVILKLANICGKACMKLLDRCKEIIVKSNVDMVSLEKSLPEELVKEIIDRRKELGLEVPKVKKHVSNVHKALDSDDIELVKLLLKEDHTNLDDACALHFAVAYCNVKTATDLLKLDLADVNHRNPRGYTVLHVAAMRKEPQLILSLLEKGASASEATLEGRTALMIAKQATMAVECNNIPEQCKHSLKGRLAVEILEQEDKREQIPRDVPPSFAVAADELKMTLLDLENRVALAQRLFPTEAQAAMEIAEMKGTCEFIVTSLEPDRLTGTKRTSPGVKIAPFRILEEHQSRLKALSKTVELGKRFFPRCSAVLDQIMNCEDLTQLACGEDDTAEKRLQKKQRYMEIQETLKKAFSEDNLELGNSSLTDSTSSTSKSTGGKRSNRKLSHRRR

SEQ ID NO: 154 - protein, 574 aa, Artificial Sequence
  Glycine max npr1 rdr2 protein
  misc_feature (166)..(166): Xaa can be any naturally occurring amino acid
MNFRSGSSDSKDASNSSTGEAYLSGVSDVITPLRRLSEQLGSILDGGGVDFFSDAKIVAGDGREVAVNRCILAARSGFFKHVFAGGGGCVLRLKEVAKDYNVGLEALGIVLAYLYSGRVKPLPQGGVCVCVDDVCSHFGCRPAIDFLLQLLYASSTFQLNELIALXQGHLLDILEKVAIDDILVVLSVANICGIVCERLLARCTEMILKSDADITTLEKALPQHLVKQITDKRIELDLYMPENFNFPDKHVNRIHRALDSDDVELVRLLLKEGHTTLDDAYALHYAVAYCDVKTTTELLDLGLADVNHKNYRGYSVLHVAAMRKEPKIIVSLLTKGAQPSDLTLDGRKALQISKRLTKAVDYYKSTEEGKVSASDRLAIEILEQAERREPLLGEASLSLAMAGDDLRMKLLYLENRVGLAKVLFPMEAKVIMDISQIDGTSEFPSTDMYCPNISDHQRTTVDLNDAPFRMKEEHLVRLRALSRTVELGKRFFPRCSEVLNKIMDADDLTQLTCMGDDSPEDRLRKRRRYVELQEVLNKVFNEDKEEFDRSAMSSSSSSTSIGVVRPNANLAMKN

SEQ ID NO: 155 - protein, 586 aa, Artificial Sequence
  Cucumis melo npr1 rdr2 protein
MADSHEPSSSLSFTSSSYTNGSQSCNMSPSSISDPMPSLEVISLNKLSSNLAQLLIHDGCDYTDADIVVEGVPVGIHRCILAVRSRFFHYLFQKDEKPAMKDGKPQYHMNELLPYGKVGHEAFLILLSYLYSGKLNSSPANVSTCVDNSCAHDACGPAIDFAVQLTYASSIFQIPELVSLFQRHLLNYVVKALVENVIQILVVAFHCQLSPLVTQCIDRIARSDLDCASLEKGLPYEVAERIKLVRLKSRGGDEQNLVADSPRDKKIKKICLALDSDDVELMKLLLSESDVTLDEANALHYAAAYCDPKSLTEVLNLNIADVNLRNSRGYTVLHVAAMRKDPSVIMSLLNKGAWAFDLTPDGRTAANIAQRLTRPKDYHAKTEKGQETNKDRLAIDILEREMWRNPTSDSSILSLAMADDVHMKLIYLENRVAFARLLFPSEARLAMDIANADTTSEFVGLSMPKNSNKNLREVNLNETPSVQNKRFLSRMQALLKTVEMGRRFFPNCSEALDKFVADDLPDLFYLEKGTVEEQRIKRKRFKELKNDVQKAFDKDKAAKLNQSGLSPSSSSTSLKHGTNHRNVRRQ

SEQ ID NO: 156 - protein, 608 aa, Artificial Sequence
  Brassica napus npr1 rdr2 protein
METIARFDDFYEISSTSFPAAPAPTNNSGSSTVFPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADESCCHVACRPAVDFMVEVLYLAFVFQIQELVTMYQRHLLDVVDKVMIEDTLVVLKLANICGKACKKLFDKCREIIVKSNVDVVTLKKSLPENIAKQVIDIRKELGLEVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAEAAILEKGKLAAKGGVAVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGTDSRGFFGIIFYMLQPSVHHLKKVSRVPPRTTTSVPSNPEGL

SEQ ID NO: 157 - protein, 579 aa, Artificial Sequence
  Brassica rapa npr1 rdr2 protein
METIAGFDDFYEISSTSFLAAPAPTDNSGSSTVYPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVNIEDTLVVLKLANICGKACKKLFDKCREIIVKSNVDVVTLKKSLPENIAKQVIDIRKELGLEVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAEAAILEKGKLAAKGGVAVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 158 - protein, 606 aa, Artificial Sequence
  Brassica oleracea npr1 rdr2 protein
METIAGFDDFYEISSTSFLAAPAPTDNSGSSTVYPTELFTRPEVSAFQLLSNSLESVFDSPEAFYSDAKLVLSDDKEVSFHRCILSARSLFFKAALTAAEKVQKSTPVKLELKTLAAEYDVGFDSVVAVLAYVYSGRVRPPPKGVSECADESCCHVACRPAVDFMVEVLYLAFVFQIQELVTMYQRHLLDVVDKVMIEDTLVVLKLANICGKACKKLFDKCREIIVKSNVDVVTLKKSLPEDIAKQVIDIRKELGLEVAEPEKHVSNIHKALESDDLDLVVMLLKEGHTNLDEAYALHFAVAYCDEKTARNLLELGFADVNRRNPRGYTVIHVAAMRKEPTLIALLLTKGANALEMSLDGRTALLIAKQVTKAAEAAILEKGKLAAKGGVAVEILKQPDNTREPFPEDVSPSLAVAADQFKIRLIDLENRVQMARCLYPMEAQVAMDFARMKGTREFVVTTATDLHMEPFKFVEMHQSRLTALSKTGIIKSFVTKITETNLCLTDLVFLLFLPVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 159 - protein, 581 aa, Artificial Sequence
  Raphanus sativus npr1 rdr2 protein
METIAGFDDFYEISSTSFLAAPAPTDNSGSSTVYPTELLTTRPEVSASQLLSNSLESVFDSPEEFYSDAKLVLSDDREVSFHRCILSARSPFFKAELAAAEKVQKSTPVKLELKKLAAEYDVGFDSVVAVLAYVYCGRVRPPPKGVSECADESCCHVACRPAVDFMVEVLYLAFVFQIPELVTMYQRHLLDVIEKVIIEDTLVVLKLANICGKACKKLFDKCKEIIVMSDVDVVTLKKSLPEDVAKQVIDIRKELGLEVAEPEKHVSNIHKALESDDLALVDMLLNEGHTNLDDAYALHFAVAYCDVQTAKDLLELELADVNRRNPRGYTVLHVAAMRKEPTLIALLLTKGANASETSLDGRTALVIAKQVTKATEAAILERGKLSAKGGVAVEILKEPDNKREPFPEDVSPSLAVAADELKIRLIDLENRVQMARCLYPMEAQVAMDFARMKGTHEFVVTTTTDLNMEPFKFVEMHRSRLTALSKTVEFGKRFFPRCSKVLDDILNFEDLTILALVEEETPEQRQQKRQRFMEIQEIVRMAFSKDKEDLGKSSLSPSSSSTSKLNGKKRSIAKLSHRRRR

SEQ ID NO: 160 - protein, 579 aa, Artificial Sequence
  Brassica juncea npr1 rdr2 protein
METIARFDDFYEISSTSFPAAPAPTDNSGSSTVYPTELLTRPEVSAFQLLSNSLESVFDSPEAFYSDAKLVLSDDKEVSFHRCILSARSLFFKAALAAAEKVQKSTPVKLELKTLAAEYDVGFDSVVAVLAYVYSGRVRPPPKGVSECADDSCCHVACRPAVDFMVEVLYLAFVFQIQELVTMYQRHLLDVVDKVNIEDTLVVLKLANICGKACKKLFDKCREIIVKSNVDVVTLKKSLPENIAKQVIDIRKELGLDVAEPEKHVSNIHKALESDDLDLVVMLLKEGHTNLDEAYALHFAVAYCDEKTARNLLELGLADVNRRNPRGYTVLHVAAMRKEPTLIALLLTKGANALETSLDGRTALLIAKQVTKAAEAAILEKGKLAAKGGVAVEILKQPDNKREPFPEDVFPSLAVAADEFKIRLIDLENRVQMARCLYPMEAQVAMDFARMKGTREFVVTTATDLHMEPFKFVEMHQSRLTALSKTVEFGKRFFPRCSKVLDDIVDSEDLTILALVEEDTPEQRQQKRQRFMEIQEIVQMAFSKDKEDLGKSSLSASSSSTSKLTGKKRSIAKPSHRRR

SEQ ID NO: 161 - protein, 13 aa, Simian virus 40
CGGGPKKKRKVED

SEQ ID NO: 162 - DNA, 48 bp, Arabidopsis thaliana
atggtcgtcgtcttcatcttcttcctccatcatcagatttttccttaa

SEQ ID NO: 163 - DNA, 111 bp, Arabidopsis thaliana
atggaagaaaccaaacgaaactccgatcttctccgttctcgtgttttcctctctggcttttattgctgggattgggaatttctcaccgctctcttgctttttagttgctga

SEQ ID NO: 164 - protein, 15 aa, Arabidopsis thaliana
MVVVFIFFLHHQIFP

SEQ ID NO: 165 - protein, 36 aa, Arabidopsis thaliana
MEETKRNSDLLRSRVFLSGFYCWDWEFLTALLLFSC

SEQ ID NO: 166 - DNA, 483 bp, Artificial Sequence
  A. thaliana TBF1 regulatory sequence with 5′ uORF1 and uORF2
tctagaaacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggag

SEQ ID NO: 167 - DNA, 3071 bp, Arabidopsis thaliana
cgacgactagtttacagagaatttggaccgtccgatgtaaagcgaaaatagatctaggttttccacgtgtcccctattttaatgaaaccttctgattcatgtagaagttttactcaatttaatattttttagtatgtagttttgtgtgtgtgtgtgtgtgtgtttttatggctccacaccaacttttaaaatggtagaagcatgttgcatgtgatcgagtaaaaagccaataatgagattcagaaaaataaaaattacttatatagttttttagagaaaaaattgtattttgtttaaagccttaatccggttgttgaaagagctgtgtcacgagttaaaaatattttcttttcattttttaagtaattagtttataatgcaaaaatggtttttatttatttgtcttcgcttatagaactgcaaattgagagagaaaaaaatgaattagtggtggtgaccaaacattcaggaagctgtgattgatcatttgtttttgaggtgagtgtagtggcaacgtatgacgttaacatatggcgtacataataattacatgaacttaatcataataatcatattgcatttaattcatatatcatatcccattagttggaccacttgatttgaggtcatgagaagaacatttatgttttttttagtttgaatcggagtgatcactaaaaactagatactgaaaattttcaaactaaaatcatattaatcttcaaaaaatgtgaaatctaaaaaaaaaaaaaattttaacgcgttcattgtagccaagtagccaagtattgttaaagtagtagtaaaagaagtttagctttaagtgatataatttgacacaaatcctacttagatatggataataggatatagcttcatgtatatttttatcgttgcttctgtaaccccaaaatgtgttgatataagcatttgaatattcgtatgtataatgttttcttttcaccgtaaaacatattacaatgttagtttatattggattttgaatgtgtttatgaacagtttttgtcgactcaaaagttaagatgagaatatggaagaaagtaaagtttaaaagtcatgatgggaacaaggaatggaactcaaacattctaatactcaacaaacgcaattatattattaccatgactcatctttcaagttccatcaaaaagattcgtggaaaataatagacttacgtttcaaatccatgtttctttctttataacaaaaaaaatggatgtttcttgacgcgtgtcgagagtactcaccattactctgacttcagtgagtttggtcaagtggtctttttttttctcatgtcaccaaaggtccaaaccctagaaattagttcgaactttccatagaagaactgaataaatggtccaaaattgttttaaaaaggacctaagccattagttcattgaattcgagttaatgggtgaagatttttatgataacgaaagtcggagtaattatgcttttggtccgatagttttctaatttgttttctttccattttttttttttcaaatactacatactatataagatagtggtttgtgttaatgtcatcgatgtgttaccatccgcattatattaattatttatcccaacataaagtcagaatctgtaatttctttgttataaaatacagtaaatggttccgtttaagctgttagatgatttttgagtaaaaactaatgtaaaaaaaacaaaaaaaaaacaatgtagttcataatacatgcatgttttaaagaagtttcttgtttactatcaacttgaatagtatttcacgaagtcaaaattgttcattccgacttttctatgtggagaaaaaaaattctatcattgtgcacaatttaacagaatgtaatttcttgtaaaagaagaggaaacaattcgctgttagtaaatgtgaagtatagaagtctaaaatgagatacctcaactagcttgaattaagaaaaaaaacaaaaactctatcgacatgaaaaaggtcgcaaatatttatcatttatcaatgccaaaggagtatttggttcacaaaatactgaatcatttatatagatatataattagctctaaattctactataacttgcaaaataagtatactgactcaattatatagcgtttaaaaatagacgatttgtatgatgaggtccatatatatggagatgtgcatgcaactatcgacattttcacacgttgatatcgtctttctccaatggagacttgaatttgtgtaaactatgaatactcgtctctctaagaccttttttcttcaaccatgccaactatttaggtaagattttactgtctttgattgatattaaatacttagccgtggcgttatcaatgaatgataataaaaatgcggataaaagccaaaggtgttggaaataaatccaagaatgaagacgtagatgtcgatgggtattttaagaacttgaatttgtcacgactcacacgttaaaatatattatccgaattgtttagtctaaagacacacatatattgaaaaagaaaaggtaaatgaagctcattggtgcctaaatgtgaaatgaagccgaaatgtgttaggtgaacacatttaaatatacaaaaagaaatataatagaaacaaaactaattaacaaagtcgcaatttgtattgtataaaatatctttccgtctcccgtcatatttgaaaaaaaaaaaattacaaatctgttaattttaaaactttctagaaaaacacaagtatataattttctcttttcgtgcgtgtttgttttaaaataacattgttttgattggcgactcaacatattttagcatttacatatttctgcatatattaaatgatttataaactcaactatagattaaaatataatttgacatctaataattttaacaataatataaaatatgagatttataaattacgaatataaatattcaagggagagaaaaagtagaacataattcaaaagataagactttttagacttttttaacaatatttttgatggataaaaattattcaaaagagaagaaagtaagaagaaaagatgtttctgagaatt

SEQ ID NO: 168 - DNA, 3554 bp, Arabidopsis thaliana
cgacgactagtttacagagaatttggaccgtccgatgtaaagcgaaaatagatctaggttttccacgtgtcccctattttaatgaaaccttctgattcatgtagaagttttactcaatttaatattttttagtatgtagttttgtgtgtgtgtgtgtgtgtgtttttatggctccacaccaacttttaaaatggtagaagcatgttgcatgtgatcgagtaaaaagccaataatgagattcagaaaaataaaaattacttatatagttttttagagaaaaaattgtattttgtttaaagccttaatccggttgttgaaagagctgtgtcacgagttaaaaatattttcttttcattttttaagtaattagtttataatgcaaaaatggtttttatttatttgtcttcgcttatagaactgcaaattgagagagaaaaaaatgaattagtggtggtgaccaaacattcaggaagctgtgattgatcatttgtttttgaggtgagtgtagtggcaacgtatgacgttaacatatggcgtacataataattacatgaacttaatcataataatcatattgcatttaattcatatatcatatcccattagttggaccacttgatttgaggtcatgagaagaacatttatgttttttttagtttgaatcggagtgatcactaaaaactagatactgaaaattttcaaactaaaatcatattaatcttcaaaaaatgtgaaatctaaaaaaaaaaaaaattttaacgcgttcattgtagccaagtagccaagtattgttaaagtagtagtaaaagaagtttagctttaagtgatataatttgacacaaatcctacttagatatggataataggatatagcttcatgtatatttttatcgttgcttctgtaaccccaaaatgtgttgatataagcatttgaatattcgtatgtataatgttttcttttcaccgtaaaacatattacaatgttagtttatattggattttgaatgtgtttatgaacagtttttgtcgactcaaaagttaagatgagaatatggaagaaagtaaagtttaaaagtcatgatgggaacaaggaatggaactcaaacattctaatactcaacaaacgcaattatattattaccatgactcatctttcaagttccatcaaaaagattcgtggaaaataatagacttacgtttcaaatccatgtttctttctttataacaaaaaaaatggatgtttcttgacgcgtgtcgagagtactcaccattactctgacttcagtgagtttggtcaagtggtctttttttttctcatgtcaccaaaggtccaaaccctagaaattagttcgaactttccatagaagaactgaataaatggtccaaaattgttttaaaaaggacctaagccattagttcattgaattcgagttaatgggtgaagatttttatgataacgaaagtcggagtaattatgcttttggtccgatagttttctaatttgttttctttccattttttttttttcaaatactacatactatataagatagtggtttgtgttaatgtcatcgatgtgttaccatccgcattatattaattatttatcccaacataaagtcagaatctgtaatttctttgttataaaatacagtaaatggttccgtttaagctgttagatgatttttgagtaaaaactaatgtaaaaaaaacaaaaaaaaaacaatgtagttcataatacatgcatgttttaaagaagtttcttgtttactatcaacttgaatagtatttcacgaagtcaaaattgttcattccgacttttctatgtggagaaaaaaaattctatcattgtgcacaatttaacagaatgtaatttcttgtaaaagaagaggaaacaattcgctgttagtaaatgtgaagtatagaagtctaaaatgagatacctcaactagcttgaattaagaaaaaaaacaaaaactctatcgacatgaaaaaggtcgcaaatatttatcatttatcaatgccaaaggagtatttggttcacaaaatactgaatcatttatatagatatataattagctctaaattctactataacttgcaaaataagtatactgactcaattatatagcgtttaaaaatagacgatttgtatgatgaggtccatatatatggagatgtgcatgcaactatcgacattttcacacgttgatatcgtctttctccaatggagacttgaatttgtgtaaactatgaatactcgtctctctaagaccttttttcttcaaccatgccaactatttaggtaagattttactgtctttgattgatattaaatacttagccgtggcgttatcaatgaatgataataaaaatgcggataaaagccaaaggtgttggaaataaatccaagaatgaagacgtagatgtcgatgggtattttaagaacttgaatttgtcacgactcacacgttaaaatatattatccgaattgtttagtctaaagacacacatatattgaaaaagaaaaggtaaatgaagctcattggtgcctaaatgtgaaatgaagccgaaatgtgttaggtgaacacatttaaatatacaaaaagaaatataatagaaacaaaactaattaacaaagtcgcaatttgtattgtataaaatatctttccgtctcccgtcatatttgaaaaaaaaaaaattacaaatctgttaattttaaaactttctagaaaaacacaagtatataattttctcttttcgtgcgtgtttgttttaaaataacattgttttgattggcgactcaacatattttagcatttacatatttctgcatatattaaatgatttataaactcaactatagattaaaatataatttgacatctaataattttaacaataatataaaatatgagatttataaattacgaatataaatattcaagggagagaaaaagtagaacataattcaaaagataagactttttagacttttttaacaatatttttgatggataaaaattattcaaaagagaagaaagtaagaagaaaagatgtttctgagaatttctagaaacagcatccgtttttataatttaattttcttacaaaggtaggaccaacatttgtgatctataaatcttcctactacgttatatagagacccttcgacataacacttaactcgtttatatatttgttttacttgttttgcacatacacacaaaaataaaaaagactttatatttatttactttttaatcacacggattagctccggcgaagtatggtcgtcgtcttcatcttcttcctccatcatcagatttttccttaaatggaagaaaccaaacgaaactccgatcttctccgttctcgtgttttcctctctggcttttattgctgggattgggaatttctcaccgctctcttgctttttagttgctgattctttttccttcgactttctatttccaatctttcttcttctctttgtgtattagattatttttagttttatttttctgtggtaaaataaaaaaagttcgccggag